(19) United States
(12) Patent Application Publication
(10) Pub. No.: US 2026/0347347 A1
(43) Pub. Date: Nov. 5, 2026

(54) 2-AZA-AND 2-OXABICYCLO[2.1.1]HEXANE DERIVATIVES AS FACTOR XIIA ENZYME INHIBITORS

(71) Applicant: KALVISTA PHARMACEUTICALS LIMITED, Wiltshire (GB)

(72) Inventors: Rebecca Louise DAVIE, Wiltshire (GB); Hannah Joy EDWARDS, Wiltshire (GB); David Michael EVANS, Wiltshire (GB); Christophe FROMONT, Wiltshire (GB); William Jack GREVES, Wiltshire (GB); Carl Leslie NORTH, Wiltshire (GB); Nicholas Paul CAMP, Okehampton Devon (GB); Johan BARTHOLOMEUS, Quebec (CA); Shawn Donald JOHNSTONE, Quebec (CA); Massaba KEITA, Quebec (CA); Julien POMAROLE, Quebec (CA)

(21) Appl. No.: 19/104,508

(22) PCT Filed: Aug. 18, 2023
(86) PCT No.: PCT/GB2023/052166

§ 371 Date: Feb. 18, 2025

Related U.S. Application Data
(60) Provisional application No. 63/399,075, filed on Aug. 18, 2022.

(30) Foreign Application Priority Data
Aug. 18, 2022 (GB) 2212081.0

(51) Int. Cl. C07D 413/14; A61K 31/4439; A61K 31/444; A61K 31/4725; A61K 31/501; A61K 31/506; A61K 31/5377; A61K 31/538; C07D 401/12; C07D 401/14; C07D 405/14; C07D 471/04; C07D 491/107

(52) U.S. Cl. CPC C07D 413/14; A61K 31/4439; A61K 31/444; A61K 31/4725; A61K 31/501; A61K 31/506; A61K 31/5377; A61K 31/538; C07D 401/12; C07D 401/14; C07D 405/14; C07D 471/04; C07D 491/107

(57) ABSTRACT

The present invention provides compounds of formula (I): Formula (I) compositions comprising such compounds; the use of such compounds in medicine; and methods of treating patients with such compounds; wherein A, W, V, Z, Y and B are as defined herein.

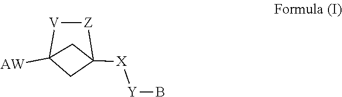

This invention relates to enzyme inhibitors that are inhibitors of Factor XIIa (FXIIa), and to pharmaceutical compositions comprising, and uses of, such inhibitors.

### BACKGROUND TO THE INVENTION

The compounds of the present invention are inhibitors of factor XIIa (FXIIa) and thus have a number of possible therapeutic applications, particularly in the treatment of diseases or conditions in which factor XIIa inhibition is implicated.

FXIIa is a serine protease (EC 3.4.21.38) derived from its zymogen precursor, factor XII (FXII), which is expressed by the F12 gene. Single chain FXII has a low level of amidolytic activity that is increased upon interaction with negatively charged surfaces and has been implicated in its activation (see Invanov et al., Blood. 2017 Mar. 16; 129(11):1527-1537. doi: 10.1182/blood-2016-10-744110). Proteolytic cleavage of FXII to heavy and light chains of FXIIa dramatically increases catalytic activity. FXIIa that retains its full heavy chain is αFXIIa. FXIIa that retains a small fragment of its heavy chain is βFXIIa. The separate catalytic activities of αFXIIa and βFXIIa contribute to the activation and biochemical functions of FXIIa. Mutations and polymorphisms in the F12 gene can alter the cleavage of FXII and FXIIa.

FXIIa has a unique and specific structure that is different from many other serine proteases. For instance, the Tyr99 in FXIIa points towards the active site, partially blocking the S2 pocket and giving it a closed characteristic. Other serine proteases containing a Tyr99 residue (e.g. FXa, tPA and FIXa) have a more open S2 pocket. Moreover, in several trypsin-like serine proteases the P4 pocket is lined by an “aromatic box” which is responsible for the P4-driven activity and selectivity of the corresponding inhibitors. However, FXIIa has an incomplete “aromatic box” resulting in more open P4 pocket. See e.g. “Crystal structures of the recombinant β-factor XIIa protease with bound Thr-Arg and Pro-Arg substrate mimetics” M. Pathak et al., Acta. Cryst. 2019, D75, 1-14; “Structures of human plasma β-factor XIIa cocrystallized with potent inhibitors” A Dementiev et al., Blood Advances 2018, 2(5), 549-558; “Design of Small-Molecule Active-Site Inhibitors of the S1A Family Proteases as Procoagulant and Anticoagulant Drugs” P. M. Fischer, J. Med. Chem., 2018, 61(9), 3799-3822; “Assessment of the protein interaction between coagulation factor XII and corn trypsin inhibitor by molecular docking and biochemical validation” B. K. Hamad et al. Journal of Thrombosis and Haemostasis, 15: 1818-1828.

FXIIa converts plasma prekallikrein (PK) to plasma kallikrein (PKa), which provides positive feedback activation of FXII to FXIIa. FXII, PK, and high molecular weight kininogen (HK) together represent the contact system. FXIIa mediated conversion of plasma prekallikrein to plasma kallikrein can cause subsequent cleavage of HK to generate bradykinin, a potent inflammatory hormone that can also increase vascular permeability, which has been implicated in disorders such as hereditary angioedema (HAE). The contact system is activated via a number of mechanisms, including interactions with negatively charged surfaces, negatively charged molecules, unfolded proteins, artificial surfaces, foreign tissue (e.g. biological transplants, that include bio-prosthetic heart valves, and organ/tissue transplants), bacteria, and biological surfaces (including endothelium and extracellular matrix) that mediate assembly of contact system components. In addition, the contact system is activated by plasmin, and cleavage of FXII by other enzymes can facilitate its activation.

Activation of the contact system leads to activation of the kallikrein kinin system (KKS), complement system, and intrinsic coagulation pathway (see https://www.genome.jp/kegg-bin/show_pathway?map04610). In addition, FXIIa has additional substrates both directly, and indirectly via PKa, including Proteinase-activated receptors (PARs), plasminogen, and neuropeptide Y (NPY) which can contribute to the biological activity of FXIIa. Inhibition of FXIIa could provide clinical benefits by treating diseases and conditions associated with these systems, pathways, receptors, and hormones.

PKa activation of PAR2 mediates neuroinflammation and may contribute to neuroinflammatory disorders including multiple sclerosis (see Göbel et al., Proc Natl Acad Sci USA. 2019 Jan. 2; 116(1):271-276. doi: 10.1073/pnas.1810020116). PKa activation of PAR1 and PAR2 on vascular smooth muscle cells has been implicated in vascular hypertrophy and atherosclerosis (see Abdallah et al., J Biol Chem. 2010 Nov. 5; 285(45):35206-15. doi: 10.1074/jbc.M110.171769). FXIIa activation of plasminogen to plasmin contributes to fibrinolysis (see Konings et al., Thromb Res. 2015 August; 136(2):474-80. doi: 10.1016/j.thromres.2015.06.028). PKa proteolytically cleaves NPY and thereby alters its binding to NPY receptors (Abid et al., J Biol Chem. 2009 Sep. 11; 284(37):24715-24. doi: 10.1074/jbc.M109.035253).

Inhibition of FXIIa could provide clinical benefits by treating diseases and conditions caused by PAR signaling, NPY metabolism, and plasminogen activation.

FXIIa-mediated activation of the KKS results in the production of bradykinin (BK), which can mediate, for example, angioedema, pain, inflammation, vascular hyperpermeability, and vasodilatation (see Kaplan et al., Adv Immunol. 2014; 121:41-89. doi: 10.1016/B978-O-12-800100-4.00002-7; and Hopp et al., J Neuroinflammation. 2017 Feb. 20; 14(1):39. doi: 10.1186/s12974-017-0815-8). Garadacimab (CSL-312), a monoclonal antibody inhibitory against FXIIa, recently completed a phase 3 study where monthly prophylactic subcutaneous treatment was reported to reduce attacks in patients with type I/II hereditary angioedema (HAE), which results in intermittent swelling of face, hands, throat, gastro-intestinal tract and genitals (see https://www.clinicaltrials.gov/ct2/show/NCT04656418 and Craig et al., The Lancet. 2023; 401(10382):1079-1090. doi: 10.1016/50140-6736(23)00350-1). Mutations in FXII that facilitate its activation to FXIIa have been identified as a cause of HAE (see Björkqvist et al., J Clin Invest. 2015 Aug. 3; 125(8):3132-46. doi: 10.1172/JC177139; and de Maat et al., J Allergy Clin Immunol. 2016 November; 138(5):1414-1423.e9. doi: 10.1016/j.jaci.2016.02.021). Since FXIIa mediates the generation of PK to PKa, inhibitors of FXIIa could provide protective effects of all form of BK-mediated angioedema, including HAE and non-hereditary bradykinin-mediated angioedema (BK-AEnH).

“Hereditary angioedema” can be defined as any disorder characterised by recurrent episodes of bradykinin-mediated angioedema (e.g. severe swelling) caused by an inherited genetic dysfunction/fault/mutation. There are currently three known categories of HAE: (i) HAE type 1, (ii) HAE type 2, and (iii) normal C1 inhibitor HAE (normal C1-Inh HAE). However, work on characterizing the etiologies of HAE is ongoing so it is expected that further types of HAE might be defined in the future.

Without wishing to be bound by theory, it is thought that HAE type 1 is caused by mutations in the SERPING1 gene that lead to reduced levels of C1 inhibitor in the blood. Without wishing to be bound by theory, it is thought that HAE type 2 is caused by mutations in the SERPING1 gene that lead to dysfunction of the C1 inhibitor in the blood. Without wishing to be bound by theory, the cause of normal C1-Inh HAE is less well defined and the underlying genetic dysfunction/fault/mutation can sometimes remain unknown. What is known is that the cause of normal C1-Inh HAE is not related to reduced levels or dysfunction of the C1 inhibitor (in contrast to HAE types 1 and 2). Normal C1-Inh HAE can be diagnosed by reviewing the family history and noting that angioedema has been inherited from a previous generation (and thus it is hereditary angioedema). Normal C1-Inh HAE can also be diagnosed by determining that there is a dysfunction/fault/mutation in a gene other than those related to C1 inhibitor. For example, it has been reported that dysfunction/fault/mutation with plasminogen can cause normal C1-Inh HAE (see e.g. Veronez et al., Front Med (Lausanne). 2019 Feb. 21; 6:28. doi: 10.3389/fmed.2019.00028; or Recke et al., Clin Transl Allergy. 2019 Feb. 14; 9:9. doi: 10.1186/s13601-019-0247-x.). It has also been reported that dysfunction/fault/mutation with Factor XII can cause normal C1-Inh HAE (see e.g. Mansi et al. 2014 The Association for the Publication of the Journal of Internal Medicine Journal of Internal Medicine, 2015, 277; 585-593; or Maat et al. J Thromb Haemost. 2019 January; 17(1):183-194. doi: 10.1111/jth.14325).

However, angioedemas are not necessarily inherited. Indeed, another class of angioedema is bradykinin mediated angioedema non-hereditary (BK-AEnH), which is not caused by an inherited genetic dysfunction/fault/mutation. Often the underlying cause of BK-AEnH is unknown and/or undefined. However, the signs and symptoms of BK-AEnH are similar to those of HAE, which, without being bound by theory, is thought to be on account of the shared bradykinin mediated pathway between HAE and BK-AEnH. Specifically, BK-AEnH is characterised by recurrent acute attacks where fluids accumulate outside of the blood vessels, blocking the normal flow of blood or lymphatic fluid and causing rapid swelling of tissues such as in the hands, feet, limbs, face, intestinal tract, airway or genitals.

Specific types of BK-AEnH include: non hereditary angioedema with normal C1 Inhibitor (AE-nC1 Inh), which can be environmental, hormonal, or drug induced; acquired angioedema; anaphylaxis associated angioedema; angiotensin converting enzyme (ACE) inhibitor induced angioedema; dipeptidyl peptidase 4 inhibitor induced angioedema; and tPA induced angioedema (tissue plasminogen activator induced angioedema). However, reasons why these factors and conditions cause angioedema in only a relatively small proportion of individuals are unknown.

Environmental factors that can induce AE-nC1 Inh include air pollution (Kedarisetty et al, Otolaryngol Head Neck Surg. 2019 Apr. 30:194599819846446. doi: 10.1177/0194599819846446) and silver nanoparticles such as those used as antibacterial components in healthcare, biomedical and consumer products (Long et al., Nanotoxicology. 2016; 10(4):501-11. doi: 10.3109/17435390.2015.1088589).

Various publications suggest a link between the bradykinin and contact system pathways and BK-AEnHs, and also the potential efficacy of treatments, see e.g.: Bas et al. (N Engl J Med 2015; Leibfried and Kovary. J Pharm Pract 2017); van den Elzen et al. (Clinic Rev Allerg Immunol 2018); Han et al (JCI 2002).

For instance, BK-medicated AE can be caused by thrombolytic therapy. For example, tPA induced angioedema is discussed in various publications as being a potentially life threatening complication following thrombolytic therapy in acute stroke victims (see e.g. Simão et al., Blood. 2017 Apr. 20; 129(16):2280-2290. doi: 10.1182/blood-2016-09-740670; Fröhlich et al., Stroke. 2019 Jun. 11:STROKEAHA119025260. doi: 10.1161/STROKEAHA.119.025260; Rathbun, Oxf Med Case Reports. 2019 Jan. 24; 2019(1):omy112. doi: 10.1093/omcr/omy112; Lekoubou et al., Neurol Res. 2014 July; 36(7):687-94. doi: 10.1179/1743132813Y.0000000302; Hill et al., Neurology. 2003 May 13; 60(9):1525-7).

Stone et al. (Immunol Allergy Clin North Am. 2017 August; 37(3):483-495.) reports that certain drugs can cause angioedema.

Scott et al. (Curr Diabetes Rev. 2018; 14(4):327-333. doi: 10.2174/1573399813666170214113856) reports cases of dipeptidyl Peptidase-4 Inhibitor induced angioedema.

Hermanrud et al., (BMJ Case Rep. 2017 Jan. 10; 2017. pii: bcr2016217802) reports recurrent angioedema associated with pharmacological inhibition of dipeptidyl peptidase IV and also discusses acquired angioedema related to angiotensin-converting enzyme inhibitors (ACEI-AAE). Kim et al. (Basic Clin Pharmacol Toxicol. 2019 January; 124(1):115-122. doi: 10.1111/bcpt.13097) reports angiotensin II receptor blocker (ARB)-related angioedema. Reichman et al., (Pharmacoepidemiol Drug Saf. 2017 October; 26(10):1190-1196. doi: 10.1002/pds.4260) also reports angioedema risk for patients taking ACE inhibitors, ARB inhibitors and beta blockers. Diestro et al. (J Stroke Cerebrovasc Dis. 2019 May; 28(5):e44-e45. doi: 10.1016/j.jstrokecerebrovasdis.2019.01.030) also reports a possible association between certain angioedemas and ARBs.

Giard et al. (Dermatology. 2012; 225(1):62-9. doi: 10.1159/000340029) reports that bradykinin mediated angioedema can be precipitated by estrogen contraception, so called “oestrogen associated angioedema”.

Contact system mediated activation of the KKS has also been implicated in retinal edema and diabetic retinopathy (see Liu et al., Biol Chem. 2013 March; 394(3):319-28. doi: 10.1515/hsz-2012-0316). FXIIa concentrations are increased in the vitreous fluid from patients with advance diabetic retinopathy and in Diabetic Macular Edema (DME) (see Gao et al., Nat Med. 2007 February; 13(2):181-8. Epub 2007 Jan. 28 and Gao et al., J Proteome Res. 2008 June; 7(6):2516-25. doi: 10.1021/pr800112g). FXIIa has been implicated in mediating both vascular endothelial growth factor (VEGF) independent DME (see Kita et al., Diabetes. 2015 October; 64(10):3588-99. doi: 10.2337/db15-0317) and VEGF mediated DME (see Clermont et al., Invest Ophthalmol Vis Sci. 2016 May 1; 57(6):2390-9. doi: 10.1167/iovs.15-18272). FXII deficiency is protective against VEGF induced retinal edema in mice (Clermont et al., ARVO talk 2019). Therefore, it has been proposed that FXIIa inhibition will provide therapeutic effects for diabetic retinopathy and retinal edema caused by retinal vascular hyperpermeability, including DME, retinal vein occlusion, age-related macular degeneration (AMD).

As noted above, the contact system can be activated by interaction with bacteria, and therefore FXIIa has been implicated in the treatment of sepsis and bacterial sepsis (see Morrison et al., J Exp Med. 1974 Sep. 1; 140(3):797-811). Therefore, FXIIa inhibitors could provide therapeutic benefits in treating sepsis, bacterial sepsis and disseminated intravascular coagulation (DIC).

FXIIa mediated activation of the KKS and production of BK have been implicated in neurodegenerative diseases including Alzheimer's disease, multiple sclerosis, epilepsy and migraine (see Zamolodchikov et al., Proc Natl Acad Sci USA. 2015 Mar. 31; 112(13):4068-73. doi: 10.1073/pnas.1423764112; Simões et al., J Neurochem. 2019 August; 150(3):296-311. doi: 10.1111/jnc.14793; Göbel et al., Nat Commun. 2016 May 18; 7:11626. doi: 10.1038/ncomms11626; and https://clinicaltrials.gov/ct2/show/NCT03108469). Therefore, FXIIa inhibitors could provide therapeutic benefits in reducing the progression and clinical symptoms of these neurodegenerative diseases.

FXIIa has also been implicated in anaphylaxis (see Bender et al., Front Immunol. 2017 Sep. 15; 8:1115. doi: 10.3389/fimmu.2017.01115; and Sala-Cunill et al., J Allergy Clin Immunol. 2015 April; 135(4):1031-43.e6. doi: 10.1016/j.jaci.2014.07.057). Therefore, FXIIa inhibitors could provide therapeutic benefits in reducing the clinical severity and incidence of anaphylactic reactions.

The role of FXIIa in coagulation was identified over 50 years ago, and has been extensively documented in publications using biochemical, pharmacological, genetic and molecular studies (see Davie et al., Science. 1964 Sep. 18; 145(3638):1310-2). FXIIa mediated activation of factor XI (FXI) triggers the intrinsic coagulation pathway. In addition, FXIIa can increase coagulation in a FXI independent manner (see Radcliffe et al., Blood. 1977 October; 50(4):611-7; and Puy et al., J Thromb Haemost. 2013 July; 11(7):1341-52. doi: 10.1111/jth.12295). Studies on both humans and experimental animal models have demonstrated that FXII deficiency prolongs activated partial prothrombin time (APTT) without adversely affecting hemostasis (see Renné et al., J Exp Med. 2005 Jul. 18; 202(2):271-81; and Simão et al., Front Med (Lausanne). 2017 Jul. 31; 4:121. doi: 10.3389/fmed.2017.00121). Pharmacological inhibition of FXIIa also prolongs APTT without increasing bleeding (see Worm et al., Ann Transl Med. 2015 October; 3(17):247. doi: 10.3978/j.issn.2305-5839.2015.09.07). These data suggest that inhibition of FXIIa could provide therapeutic effects against thrombosis without inhibiting bleeding. Therefore, FXIIa inhibitors could be used to treat a spectrum of prothrombotic conditions including venous thromboembolism (VTE); cancer associated thrombosis; complications caused by mechanical and bioprosthetic heart valves, catheters, extracorporeal membrane oxygenation (ECMO), left ventricular assisted devices (LVAD), dialysis, cardiopulmonary bypass (CPB); sickle cell disease, joint arthroplasty, thrombosis induced by tPA, Paget-Schroetter syndrome and Budd-Chari syndrome. FXIIa inhibitor could be used for the treatment and/or prevention of thrombosis, edema, and inflammation associated with these conditions.

Surfaces of medical devices that come into contact with blood can cause thrombosis. FXIIa inhibitors may also be useful for treating or preventing thromboembolism by lowering the propensity of devices that come into contact with blood to clot blood. Examples of devices that come into contact with blood include vascular grafts, stents, in-dwelling catheters, external catheters, orthopedic prosthesis, cardiac prosthesis, and extracorporeal circulation systems.

Preclinical studies have shown that FXIIa has been shown to contribute to stroke and its complications following both ischemic stroke, and hemorrhagic accidents (see Barbieri et al., J Pharmacol Exp Ther. 2017 March; 360(3):466-475. doi: 10.1124/jpet.116.238493; Krupka et al., PLoS One. 2016 Jan. 27; 11(1):e0146783. doi: 10.1371/journal.pone.0146783; Leung et al., Transl Stroke Res. 2012 September; 3(3):381-9. doi: 10.1007/s12975-012-0186-5; Simso et al., Blood. 2017 Apr. 20; 129(16):2280-2290. doi: 10.1182/blood-2016-09-740670; and Liu et al., Nat Med. 2011 February; 17(2):206-10. doi: 10.1038/nm.2295). Therefore, FXIIa inhibition may improve clinical neurological outcomes in the treatment of patients with stroke.

FXII deficiency has been shown to reduce the formation of atherosclerotic lesions in Apoe^{-/-} mice (Didiasova et al., Cell Signal. 2018 November; 51:257-265. doi: 10.1016/j.cellsig.2018.08.006). Therefore, FXIIa inhibitors could be used in the treatment of atherosclerosis.

FXIIa, either directly, or indirectly via PKa, has been shown to activate the complement system (Ghebrehiwet et al., Immunol Rev. 2016 November; 274(1):281-289. doi: 10.1111/imr.12469). BK increases complement C3 in the retina, and an in vitreous increase in complement C3 is associated with DME (Murugesan et al., Exp Eye Res. 2019 Jul. 24; 186:107744. doi: 10.1016/j.exer.2019.107744). Both FXIIa and PKa activate the complement system (see Irmscher et al., J Innate Immun. 2018; 10(2):94-105. doi: 10.1159/000484257; and Ghebrehiwet et al., J Exp Med. 1981 Mar. 1; 153(3):665-76).

A phase 2 study to assess the safety and efficacy of CSL312, a FXIIa inhibitor, in the treatment of COVID-19 has been assigned clinicaltrials.gov identifier NCT04409509. Shatzel et al. (Res Pract Thromb Haemost, 2020 May 15; 4(4):500-505. doi: 10.1002/rth2.12349) also relates to investigating the contact system's role in COVID-19.

Wygrecka et al. (“Coagulation factor XII regulates inflammatory responses in human lungs”, European Respiratory Journal 2017 50: PA339; DOI: 10.1183/1393003.congress-2017.PA339) relates to the effect of an accumulation of FXII in acute respiratory distress syndrome (ARDS) lungs.

Wong et al. (“CSL312, a Novel Anti-FXII Antibody, Blocks FXII-Induced IL-6 Production from Primary Non-Diseased and Idiopathic Pulmonary Fibrosis Fibroblasts”, American Journal of Respiratory and Critical Care Medicine 2020; 201:A6363) reports that activated FXII may contribute to lung fibrosis (e.g. idiopathic Pulmonary Fibrosis) through direct stimulation of fibroblasts to produce pro-fibrotic cytokine IL-6.

Göbel et al. (The Coagulation Factors Fibrinogen, Thrombin, and Factor XII in Inflammatory Disorders—A Systematic Review, Front. Immunol., 26 Jul. 2018| https://doi.org/10.3389/fimmu.2018.01731) relates to FXII's role in the rheumatoid arthritis (RA).

Scheffel et al. (Cold-induced urticarial autoinflammatory syndrome related to factor XII activation, Nature Communications volume 11, Article number: 179 (2020)) reports that there is a link between contact system activation and cytokine-mediated inflammation, such as cold-induced urticarial autoinflammatory syndrome.

Peyrou et al. (The kallikrein-kinin pathway as a mechanism for auto-control of brown adipose tissue activity, NATURE COMMUNICATIONS, (2020) 11:2132, https://doi.org/10.1038/s41467-020-16009-x), reports a pathway for controlling brown adipose tissue (BAT) thermogenic activity mediated by the kallikrein-kinin system, which may contribute to expanding the range of potential pharmacological candidates in therapeutic strategies against obesity and associated diseases designed to improve energy expenditure and remove excess blood metabolites through activation of BAT. Impaired BAT activity is associated with obesity and insulin resistance.

Wygrecka et al. (“Use of a FXIIa-Inhibitor in the treatment of renal fibrosis and/or kidney disease” WO2019/113642) relates to an inhibitor of Factor XII for use in treating or preventing kidney disease, renal fibrosis, glomerulosclerosis, renal scarring, ischemia/reperfusion injury in native or transplant kidneys and/or acute kidney injury.

Compounds that are said to be FXIIa inhibitors have been described by Rao et al. (“Factor XIIa Inhibitors” WO2018/093695), Hicks et al. (“Factor XIIa Inhibitors” WO2018/093716), Breslow et al. (“Aminotriazole immunomodulators for treating autoimmune diseases” WO2017/123518) and Ponda et al. (“Aminacylindazole immunomodulators for treatment of autoimmune diseases” WO2017/205296 and “Pyranopyrazole and pyrazolopyridine immunomodulators for treatment of autoimmune diseases” WO2019/108565). FXII/FXIIa inhibitors are said to have been described by Nolte et al. (“Factor XII inhibitors for the administration with medical procedures comprising contact with artificial surfaces” WO2012/120128).

Compounds that are said to be modulators of FXIIa have been described by Philippou et al. (“Factor XIIa Inhibitors” WO 2019/211585 and WO 2019/186164). Macrocylic peptides that are said to be inhibitors of FXIIa have been described by Wilbs et al. (Nat Commun 11, 3890 (2020). Doi: 10.1038/s41467-020-17648-w).

To date, no FXIIa inhibitors have been approved for medical use, and there are no small molecule FXIIa inhibitors in clinical development. Although certain known compounds are said to be modulators or inhibitors of FXIIa, these compounds can suffer from limitations such as being non-reversible or covalent binders, being poorly selective for FXIIa over other related enzymes, or not having pharmacokinetic properties suitable for oral therapy. For example, compounds with acylating reactivity e.g. acylated aminotriazoles, are typically non-reversible covalent binders, and can sometimes also be unstable in water and/or blood plasma due to their inherent reactivity. Poor selectivity for FXIIa over other serine proteases (such as thrombin, FXa, FXIa, KLK1, plasmin, trypsin) increases the risk of off-target effects, which can be made even worse (i.e. there is typically a higher likelihood of poor selectivity and off-target effects) if the inhibitor is a covalent binder. Therefore, there remains a need to develop new FXIIa inhibitors that are not covalent inhibitors and/or are highly selective for FXIIa in order to e.g. mitigate the risks of non-selectivity and cytotoxicity. There is a particular need to develop a small molecule FXIIa inhibitors, particularly as an oral therapy.

In view of the above, there also remains a need to develop new FXIIa inhibitors that will have utility to treat a wide range of disorders, in particular angioedema; HAE, including: (i) HAE type 1, (ii) HAE type 2, and (iii) normal C1 inhibitor HAE (normal C1-Inh HAE); BK-AEnH, including AE-nC1 Inh, ACE and tPA induced angioedema; vascular hyperpermeability; stroke including ischemic stroke and haemorrhagic accidents; retinal edema; diabetic retinopathy; impaired visual acuity; DME; retinal vein occlusion; AMD; neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; anaphylaxis; thrombosis; thromboembolism caused by increased propensity of medical devices that come into contact with blood to clot blood; prothrombotic conditions including disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; acute respiratory distress syndrome (ARDS); idiopathic pulmonary fibrosis (IPF); rheumatoid arthritis (RA); cold-induced urticarial autoinflammatory syndrome; obesity; diabetes; kidney disease; renal fibrosis; glomerulosclerosis; renal scarring; ischemia/reperfusion injury in native or transplant kidneys; and acute kidney injury. In particular, there remains a need to develop new FXIIa inhibitors.

### DESCRIPTION OF THE INVENTION

The present invention relates to a series of inhibitors of Factor XIIa (FXIIa). The compounds of the invention are potentially useful in the treatment of diseases or conditions in which factor XIIa inhibition is implicated. The invention further relates to pharmaceutical compositions of the inhibitors, to the use of the compositions as therapeutic agents, and to methods of treatment using these compositions.

Specifically, the invention provides compounds of Formula (I),

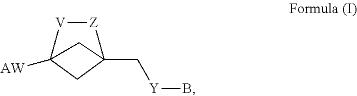

wherein:
V is selected from —O— and —N(R23)-, and Z is selected from —C(R16)(R17)-CH₂— and —C(R16)(R17)-; or,
V is selected from —CH₂—C(R16)(R17)- and —C(R16)(R17)-, and Z is selected from —O— and —N(R18)-;
   wherein R18 or R23 are selected from C(═S)NH-aryl; C(═S)NH—R19; C(═S)N(alkyl)R19; C(═S)NR13R14; C(═O)R19; C(═O)NHR19; C(═O)N(alkyl)(R19); C(═O)NH₂;
   heterocycloalkyl^{b}; a 5- or 6-membered carbon-containing aromatic ring containing one, two or three ring members independently selected from N and, where possible, O, wherein the carbon-containing aromatic ring is substituted with one substituent selected from cycloalkyl and heterocycloalkyl^{b}, and optionally one or two further substituents independently selected from alkyl, alkoxy, OH, OCF₃, halo, CN, and CF₃;

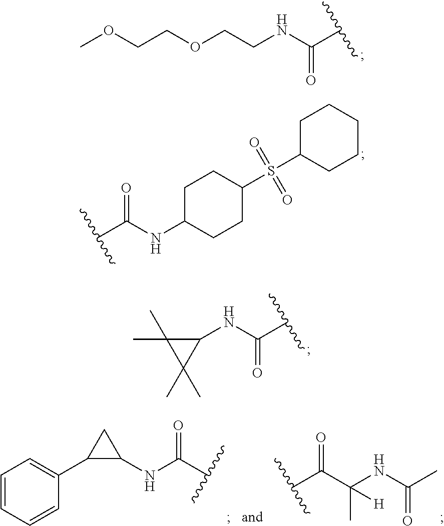

R19 is selected from (CH₂)₁₋₃-cycloalkyl, (CH₂)₀₋₃-polycycloalkyl, heterocycloalkyl^{a}, (CH₂)₁₋₃-heterocycloalkyl^{b}, (CH₂)₀₋₃—R22, (C(R20)(R21))-(CH₂)₀₋₃-phenyl, (C(R20)(R21))-R22, (C(R20)(R21))-O-phenyl, and (CH₂)₁₋₃—O—(CH₂)-phenyl;
R20 and R21 are independently selected from H, alkyl and CF₃, or R20 and R21 together with the carbon atom to which they are attached form a carbon-containing 3-, 4-, 5- or 6-membered saturated ring, optionally containing an O ring member, wherein R20 and R21 are not both H;
R22 is a fused 6,5- or 6,6-bicyclic ring containing an aromatic ring fused to a non-aromatic ring, wherein the bicyclic ring optionally contains one or two ring members selected from N and O, wherein the fused 6,5- or 6,6-bicyclic ring may be optionally substituted with one or two substituents independently selected from alkyl, alkoxy, OH, CN, CF₃, halo, and oxo, wherein the 6,5-bicyclic ring may be attached via the 6- or 5-membered ring, wherein the 6,5-bicyclic ring is not aryl substituted with methylenedioxy, and wherein the 6,6bicyclic ring is not aryl substituted with ethylenedioxy;
Y is selected from —O— and —N(R12)-;
B is selected from:
   (i) heteroaryl^{a}; and
   (ii) aryl;
AW— is selected from:
—(CH₂)₀₋₆—(CHR15)-(CH₂)₀₋₆-A, —(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—N(R12)-(CH₂)₁₋₆—C(═O)-A, —(CH₂)₀₋₆—NH—C(═O)—(CH₂)₀₋₆-A, —C(═O)N(R12)-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A and —SO₂—NH-A;
A is a 4- to 15-membered mono-, bi-, or tri-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN, and phenyl;
wherein when A is a bicyclic ring system, the bicyclic ring system is fused, bridged or spiro;
wherein when A is a tricyclic ring system, each of the three rings in the tricyclic ring system is either fused, bridged or spiro to at least one of the other rings in the tricyclic ring system;
wherein when:
(i) V is —O—, or
(ii) Z is —O—,
A is A′, wherein A′ is a 4- to 15-membered mono-, bi-, or tri-cyclic ring system, containing one N ring member and optionally one, two or three further ring members independently selected from N, O and S, wherein the ring system is substituted with phenyl and optionally, where possible, 1, 2, or 3 further substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN, and phenyl;
wherein when A′ is a bicyclic ring system, the bicyclic ring system is fused, bridged or spiro;
wherein when A′ is a tricyclic ring system, each of the three rings in the tricyclic ring system is either fused, bridged or spiro to at least one of the other rings in the tricyclic ring system;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted, where possible, with 1 or 2 substituents independently selected from OH, CN, CF₃, and fluoro;
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from alkoxy, OH, —NR13R14, —C(═O)OR13, —C(═O)NR13R14, CN, CF₃, halo;
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from alkoxy, OH, CN, CF₃, halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from alkoxy, OH, NR13R14, C(═O)OR13, C(═O)NR13R14, CN, CF₃, halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—O-heteroaryl^{a}, aryl^{b}, —O-aryl^{b}, —(CH₂)₁₋₃-aryl^{b}, —(CH₂)₀₋₃-heteroaryl^{a}, —C(═O)OR13, —C(═O)NR13R14, —(CH₂)₀₋₃—NR13R14, OCF₃ and CF₃;
aryl^{b} is phenyl, biphenyl or naphthyl; aryl^{b} may be optionally substituted with 1, 2 or 3 substituents independently selected from alkyl^{b}, alkoxy, OH, halo, CN, and CF₃;
cycloalkyl is a monocyclic saturated hydrocarbon ring of between 3 and 6 carbon atoms (C₃-C₆); cycloalkyl may optionally be substituted with 1 or 2 substituents independently selected from alkyl, alkoxy, OH, CN, CF₃, halo;
halo is F, Cl, Br, or I;
heteroaryl is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃;
heteroaryl^{a} is a 5-, 6-, 9- or 10-membered mono- or bi-cyclic aromatic ring, containing, where possible, 1, 2, 3 or 4 ring members independently selected from N, N(R12), S and O; heteroaryl^{a} may be optionally substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃;
heteroaryl^{b} is a 5-, 6-, 9- or 10-membered mono- or bi-cyclic aromatic ring, containing, where possible, 1, 2 or 3 ring members independently selected from N, N(R12), S and O; wherein heteroaryl^{b} may be optionally substituted with 1, 2 or 3 substituents independently selected from alkyl^{b}, alkoxy, OH, halo, CN, aryl^{b}, —(CH₂)₁₋₃-aryl^{b}, and CF₃;
heterocycloalkyl is a non-aromatic carbon-containing monocyclic ring containing 3, 4, 5, or 6, ring members, wherein at least one ring member is independently selected from N, N(R12), S, and O; heterocycloalkyl may optionally be substituted with 1 or 2 substituents independently selected from alkyl, alkoxy, OH, CN, CF₃, halo;
heterocycloalkyl^{a} is a 3-10 membered non-aromatic carbon-containing mono- or bi-cyclic ring system containing one or two ring members independently selected from N and O; heterocycloalkyl^{a} may optionally be substituted with 1 or 2 substituents independently selected from alkyl, alkoxy, aryl, halo, OH, CN, CF₃, and heterocycloalkyl; wherein when heterocycloalkyl^{a} is a bicyclic ring system, the bicyclic ring system is spiro;
heterocycloalkyl^{b} is a 3-6 membered non-aromatic carbon-containing monocyclic ring system which may be saturated or, where possible, unsaturated with 1 or 2 double bonds; heterocycloalkyl^{b} contains one or two ring members independently selected from N, N(C(═O)CH₃) and O; heterocycloalkyl^{b} may optionally be substituted with 1 or 2 substituents independently selected from alkyl, alkoxy, halo, OH, CN, CF₃, phenyl and oxo; wherein the point of attachment of heterocycloalkyl^{b} is not at a ring nitrogen;
polycycloalkyl is a bi- or tri-cyclic saturated hydrocarbon ring system of between 5 and 10 carbon atoms (C₅-C₁₀), wherein the bi- or tri-cyclic ring system is bridged or spiro;
R8 is independently selected from H, alkyl, cycloalkyl, and heterocycloalkyl;
R12 is independently selected from H, alkyl, and cycloalkyl;
R13 and R14 are independently selected from H, alkyl^{b}, aryl^{b} and heteroaryl^{b} or R13 and R14 together with the nitrogen atom to which they are attached form a carbon-containing 4-, 5-, 6- or 7-membered heterocyclic ring, optionally containing an additional heteroatom selected from N, N(R12), S, SO, SO₂, and O, which may be saturated or unsaturated with 1 or 2 double bonds and which may be optionally mono- or di-substituted with substituents selected from oxo, alkyl^{b}, alkoxy, OH, halo and CF₃;
R15 is selected from alkyl, halo, CF₃, CN, OH, alkoxy, NR13R14, and CONR13R14;
R16 and R17 are independently selected from H and small alkyl;
and tautomers, isomers, stereoisomers (including enantiomers, diastereoisomers and racemic and scalemic mixtures thereof), deuterated analogues, and pharmaceutically acceptable salts and/or solvates thereof.

The compounds of formula (I) have been developed to be inhibitors of FXIIa, which as noted above, has a unique and specific binding site. The compounds of formula (I) aim to address the need for small molecule FXIIa inhibitors, particularly with pharmacokinetic properties suitable for use as oral therapies. The compounds of formula (I) can also avoid including groups associated with covalent binding properties e.g. groups with acylating reactivity such as acylated aminotriazoles, and thus can provide compounds that are reversible inhibitors, to further reduce the risk of off-target effects and cytotoxicity.

The present invention also provides a prodrug of a compound as herein defined, or a pharmaceutically acceptable salt and/or solvate thereof.

The present invention also provides an N-oxide of a compound as herein defined, or a prodrug or pharmaceutically acceptable salt and/or solvate thereof.

It will be understood that “methylenedioxy” refers to —O—CH₂—O— only, wherein “methylenedioxy” cannot be substituted.

It will be understood that “ethylenedioxy” refers to —O—CH₂—CH₂—O— only, wherein “ethylenedioxy” cannot be substituted.

It will be understood that “aryl substituted with methylenedioxy” refers to aryl as defined above, wherein aryl is substituted with —O—CH₂—O—. For example,

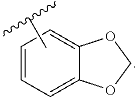

It will be understood that “aryl substituted with ethylenedioxy” refers to aryl as defined above, wherein aryl is substituted with —O—CH₂—CH₂—O—. For example,

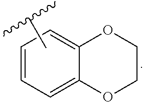

It will be understood that, as noted above R22 is not aryl substituted with methylenedioxy, which as mentioned above excludes

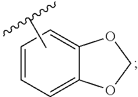

and R22 is not aryl substituted with ethylenedioxy which as mentioned above excludes

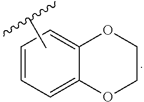

R22 can include fused 6,5- or 6,6-bicyclic rings containing an aryl ring fused to a non-aromatic ring, wherein the non-aromatic ring contains two O ring members, positioned as for

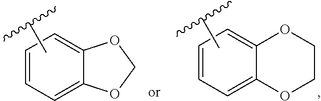

but wherein the non-aromatic ring is substituted as for R22. For example, R22 can be

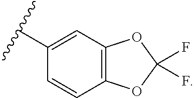 [SMILES: *c1ccc2c(c1)OC(F)(F)O2]

It will be understand that A and A′, as defined above, contain at least one N ring member. Other than optional non-carbon ring members, it will be understood that the remaining ring members are carbon.

It will be understood that “pharmaceutically acceptable salts and/or solvates thereof” means “pharmaceutically acceptable salts thereof”, “pharmaceutically acceptable solvates thereof”, and “pharmaceutically acceptable solvates of salts thereof”.

The compounds of the present invention can be provided as mixtures of more than one stereoisomer. When provided as a mixture of stereoisomers, one stereoisomer can be present at a purity >90% relative to the remaining stereoisomers. More specifically, when provided as a mixture of stereoisomers, one stereoisomer can be present at a purity >95% relative to the remaining stereoisomers.

It will be understood that substituents may be named as its free unbonded structure (e.g. piperidine) or by its bonded structure (e.g. piperidinyl). No difference is intended.

It will be understood that the compounds of the invention comprise several substituents. When any of these substituents is defined more specifically herein, the substituents/optional substituents to these groups described above also apply, unless stated otherwise. For example, B can be heteroaryl^{a}, which more specifically can be isoquinolinyl. In this case, isoquinolinyl can be optionally substituted in the same manner as “heteroaryl^{a}”.

It will be understood that the term “where possible” means that the group, atom, or substituent in question may be present if it is chemically possible to do so, e.g. does not exceed the valencies of chemically stable compounds.

It will be understood that a fused ring system refers to a ring system where two rings in the ring system share two adjacent atoms (i.e., one common covalent bond). For example,

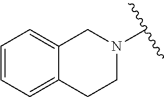 [SMILES: *N1CCc2ccccc2C1]

is a fused ring system (specifically a fused bicyclic ring system) which can be considered as a benzene ring and a piperidine ring sharing a common bond.

It will be understood that a bridged ring system refers to a ring system having two rings sharing three or more atoms. For example,

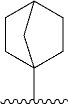 [SMILES: *C12CCC(CC1)C2]

is a bridged ring system (specifically a bridged bicyclic ring system) which can be considered as a cyclopentane ring and a second cyclopentane ring joined at a bridge and sharing three common atoms.

It will be understood that a spiro ring system refers to a ring system where two rings in the ring system share one common atom. For example,

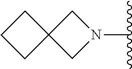 [SMILES: *N1CC2(CCC2)C1]

is a spiro ring system (specifically a spiro bicyclic ring system) which can be considered as a cyclobutane ring and an azetidine ring sharing a common carbon atom.

It will be understood that the ring system A, as defined in formula (I), can be fully saturated, or have any degree of unsaturation. For example, the ring system can be fully saturated, partially unsaturated, aromatic, non-aromatic, or have an aromatic ring bridged, fused or spiro to a non-aromatic ring.

It will be understood that ring system A can contain non-carbon ring members, and that these non-carbon ring members can, where possible, be optionally substituted themselves (as well, or as opposed to the carbon ring members), with the optional substituents included in the definition of A.

It will be understood that when any variable (e.g. alkyl) occurs more than once, its definition on each occurrence is independent of every other occurrence.

It will be understood that combinations of substituents and variables are permissible only if such combinations result in stable compounds.

As used herein the term “bradykinin-mediated angioedema” means hereditary angioedema, and any non-hereditary bradykinin-mediated angioedema. For example, “bradykinin-mediated angioedema” encompasses hereditary angioedema and acute bradykinin-mediated angioedema of unknown origin.

As used herein, the term “hereditary angioedema” means any bradykinin-mediated angioedema caused by an inherited genetic dysfunction, fault, or mutation. As a result, the term “HAE” includes at least HAE type 1, HAE type 2, and normal C1 inhibitor HAE (normal C1-Inh HAE).

More specifically, the invention provides compounds of formula (I) wherein the compound is a compound of formula (Ia), formula (Ib), or formula (Ic),

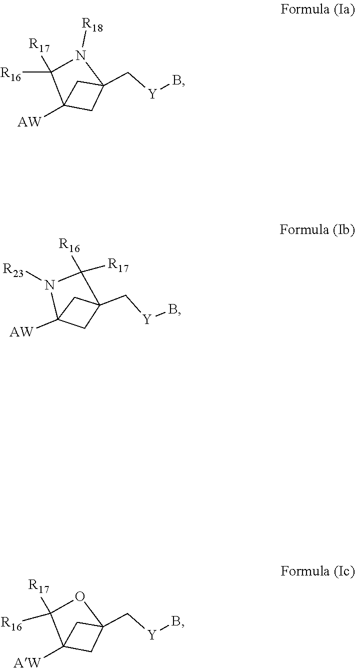

or a tautomer, isomer, stereoisomer (including an enantiomer, a diastereoisomer and a racemic and scalemic mixture thereof), a deuterated analogue, and a pharmaceutically acceptable salt and/or solvate thereof.

The invention provides compounds of formula (I) wherein the compound is a compound of formula (Ia),

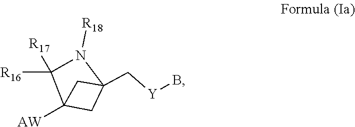

or a tautomer, isomer, stereoisomer (including an enantiomer, a diastereoisomer and a racemic and scalemic mixture thereof), a deuterated analogue, and a pharmaceutically acceptable salt and/or solvate thereof.

The invention provides compounds of formula (I) wherein the compound is a compound of formula (Ib),

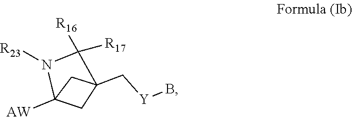

or a tautomer, isomer, stereoisomer (including an enantiomer, a diastereoisomer and a racemic and scalemic mixture thereof), a deuterated analogue, and a pharmaceutically acceptable salt and/or solvate thereof.

The invention provides compounds of formula (I) wherein the compound is a compound of formula (Ic),

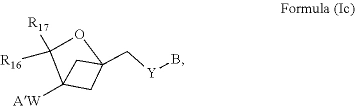

or a tautomer, isomer, stereoisomer (including an enantiomer, a diastereoisomer and a racemic and scalemic mixture thereof), a deuterated analogue, and a pharmaceutically acceptable salt and/or solvate thereof.

As noted above, B can be selected from:
(i) heteroaryl^{a}; and
(ii) aryl.

B can be aryl; or B can be a 6-membered monocyclic or 9- or 10-membered bicyclic aromatic ring, containing, where possible, 1, 2, 3 or 4 ring members independently selected from N, N(R12), S and O, optionally substituted as for heteroaryl^{a}.

B can be aryl; or B can be 6-membered monocyclic or 10-membered bicyclic aromatic ring, containing, where possible, 1, 2, 3 or 4 ring members independently selected from N, N(R12), S and O, optionally substituted as for heteroaryl^{a}.

B can be aryl; or B can be 6-membered monocyclic or 10-membered bicyclic aromatic ring, containing 1 or 2 ring members independently selected from N or N(R12), optionally substituted as for heteroaryl^{a}.

B can be phenyl substituted as for aryl; or B can be a 6-membered monocyclic or 9- or 10-membered bicyclic aromatic ring, containing, where possible, 1, 2, 3 or 4 ring members independently selected from N, N(R12), S and O, optionally substituted as for heteroaryl^{a}.

B can be phenyl substituted as for aryl; or B can be a 6-membered monocyclic or 10-membered bicyclic aromatic ring, containing, where possible, 1, 2, 3 or 4 ring members independently selected from N, N(R12), S and O, optionally substituted as for heteroaryl^{a}.

B can be phenyl substituted as for aryl; or B can be a 6-membered monocyclic or 10-membered bicyclic aromatic ring, containing 1 or 2 ring members independently selected from N or N(R12), optionally substituted as for heteroaryl^{a}.

B can be phenyl substituted with CN and optionally 1 or 2 further substituents as for aryl; or B can be a 6-membered monocyclic or 9- or 10-membered bicyclic aromatic ring, containing, where possible, 1, 2, 3 or 4 ring members independently selected from N, N(R12), S and O, optionally substituted as for heteroaryl^{a}.

B can be phenyl substituted with CN and optionally 1 or 2 further substituents as for aryl; or B can be a 6-membered monocyclic or 10-membered bicyclic aromatic ring, containing, where possible, 1, 2, 3 or 4 ring members independently selected from N, N(R12), S and O, optionally substituted as for heteroaryl^{a}.

B can be phenyl substituted with CN and optionally 1 or 2 further substituents as for aryl; or B can be a 6-membered monocyclic or 10-membered bicyclic aromatic ring, containing 1 or 2 ring members independently selected from N or N(R12), optionally substituted as for heteroaryl^{a}.

B can be phenyl optionally substituted as for aryl, or B can be pyridyl or isoquinolinyl, optionally substituted as for heteroaryl^{a}.

B can be phenyl substituted with CN and optionally 1 or 2 further substituents as for aryl, or B can be pyridyl or isoquinolinyl, optionally substituted as for heteroaryl^{a}. B can be

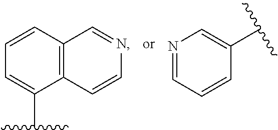 [SMILES: *c1cccc2cnccc12 ; "or" ; *c1cccnc1]

or optionally substituted as for heteroaryl^{a}, or B can be phenyl optionally substituted as for aryl. B can be

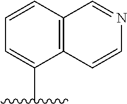 [SMILES: *c1cccc2cnccc12]

optionally substituted as for heteroaryl^{a}. B can be

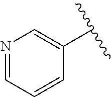 [SMILES: *c1cccnc1]

optionally substituted as for heteroaryl^{a}. B can be phenyl optionally substituted as for aryl. B can be

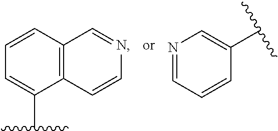 [SMILES: *c1cccc2cnccc12 ; "or" ; *c1cccnc1]

or optionally substituted as for heteroaryl^{a}, or B can be phenyl substituted with CN and optionally 1 or 2 further substituents as for aryl. B can be phenyl substituted with CN and optionally 1 or 2 further substituents as for aryl.

B can be unsubstituted; or B can be aryl, wherein B can be substituted with CN and optionally 1 or 2 further substituents as for aryl; or B can be heteroaryl^{a}, wherein B can be substituted with alkyl, halo, OH, or NH₂, and optionally 1 or 2 further substituents as for heteroaryl^{a}. B can be selected from

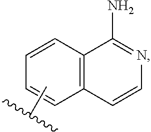

optionally further substituted with 1 or 2 substituents as for heteroaryl^{a};

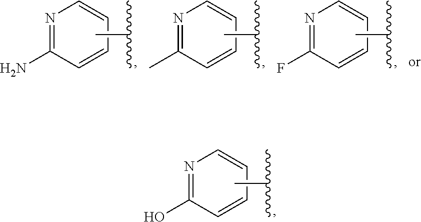

each optionally further substituted with 1 or 2 substituents as for heteroaryl^{a};

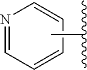

with no substituents; or

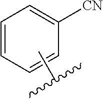

with no further substituents. B can be

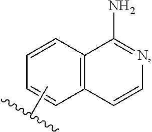

optionally further substituted with 1 or 2 substituents as for heteroaryl^{a}. B can be

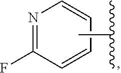

optionally further substituted with 1 or 2 substituents as for heteroaryl^{a}. B can be

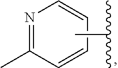

optionally further substituted with 1 or 2 substituents as for heteroaryl^{a}. B can be

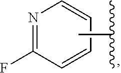

optionally further substituted with 1 or 2 substituents as for heteroaryl^{a}. B can be

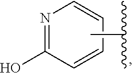

optionally further substituted with 1 or 2 substituents as for heteroaryl^{a}. B can be

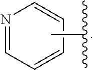

### B can be

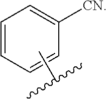

B can be selected from the following groups:

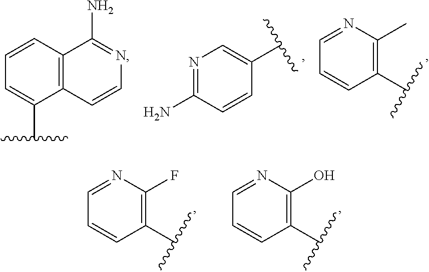

optionally further substituted with 1 or 2 substituents as for heteroaryl^{a}; or B can be

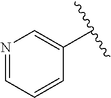 [SMILES: *c1cccnc1]

with no substituents; or B can be

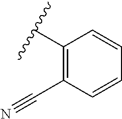 [SMILES: *c1ccccc1C#N]

with no further substituents. B can be

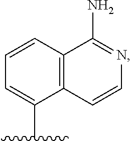 [SMILES: *c1cccc2c(N)nccc12]

optionally further substituted with 1 or 2 substituents as for heteroaryl^{a}. B can be

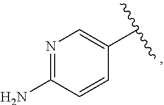 [SMILES: *c1ccc(N)nc1]

optionally further substituted with 1 or 2 substituents as for heteroaryl^{a}. B can be

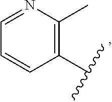 [SMILES: *c1cccnc1C]

optionally further substituted with 1 or 2 substituents as for heteroaryl^{a}. B can be

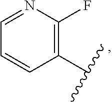 [SMILES: *c1cccnc1F]

optionally further substituted with 1 or 2 substituents as for heteroaryl^{a}. B can be

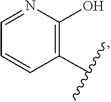 [SMILES: *c1cccnc1O]

optionally further substituted with 1 or 2 substituents as for heteroaryl^{a}. B can be

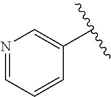 [SMILES: *c1cccnc1]

with no substituents. B can be

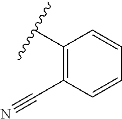 [SMILES: *c1ccccc1C#N]

with no further substituents.

B can be selected from

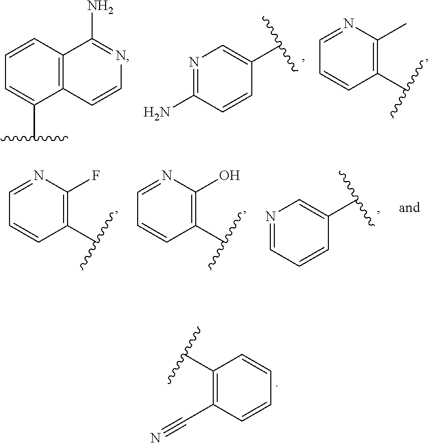

### B can be

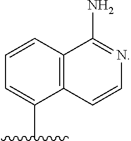 [SMILES: *c1cccc2c(N)nccc12]

### B can be

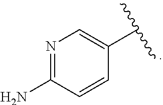 [SMILES: *c1ccc(N)nc1]

### B can be

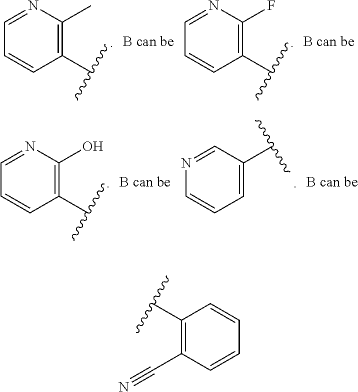

Preferably, B is heteroaryl^{a}.

B can be heteroaryl^{a} and Y can be attached to B at a carbon atom on the heteroaryl^{a} ring.

B can be heteroaryl^{a} and Y can be attached to B at a carbon atom on the heteroaryl^{a} ring, and the two ring atoms adjacent to the carbon atom on the heteroaryl^{a} ring to which Y attaches are both carbon.

It will be understood that, in the instance when Y is attached to B at a carbon atom on the heteroaryl^{a} ring, the attachment of Y to B can be at any carbon on the heteroaryl^{a} ring, so long as the remainder of the ring is still a heteroaryl ring.

It will be understood that, in the instance when Y is attached to B at a carbon atom on the heteroaryl^{a} ring, and the two ring atoms adjacent to the carbon atom on the heteroaryl^{a} ring to which Y attaches are both carbon, these adjacent ring atoms can be, where possible, substituted or unsubstituted as defined in the embodiment or claim. Further, for example, if B is isoquinolinyl, the attachment to Y can be at any of the following ring atoms:

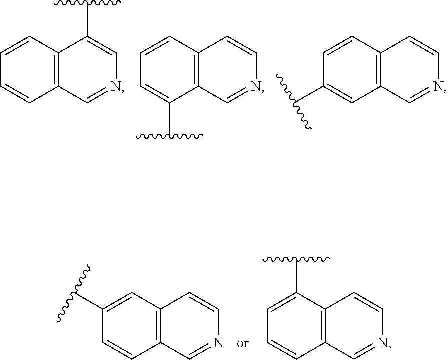

but not at the following ring atoms:

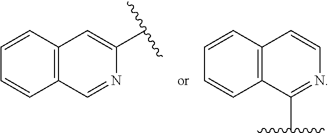 [SMILES: *c1cc2ccccc2cn1 ; *c1nccc2ccccc12]

When B is heteroaryl^{a}, B can be a 9 or 10-membered bicyclic aromatic ring, containing, where possible, 1, 2, 3 or 4 ring members independently selected from N, N(R12), S and O, optionally substituted as for heteroaryl^{a}. B can be a 10-membered bicyclic aromatic ring, containing, where possible, 1, 2, 3 or 4 ring members independently selected from N, N(R12), S and O, optionally substituted as for heteroaryl^{a}. B can be a 10-membered bicyclic aromatic ring, containing 1 or 2 ring members independently selected from N or N(R12), optionally substituted as for heteroaryl^{a}.

When B is heteroaryl^{a}, B is preferably isoquinolinyl, optionally substituted as for heteroaryl^{a}.

When B is heteroaryl^{a}, B is preferably isoquinolinyl, wherein Y is attached to B at a carbon atom on the heteroaryl^{a} ring, and the two ring atoms adjacent to the carbon atom on the heteroaryl^{a} ring to which Y attaches are both carbon.

When B is isoquinolinyl, B is preferably

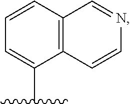 [SMILES: *c1cccc2cnccc12]

optionally substituted as for heteroaryl^{a}.

When B is heteroaryl^{a}, B is preferably substituted with NH₂, and optionally substituted with 1 or 2 further substituents as for heteroaryl^{a}.

When B is isoquinolinyl, B is preferably substituted with NH₂, and optionally substituted with 1 or 2 further substituents as for heteroaryl^{a}.

When B is isoquinolinyl, B can be substituted with NH₂ at the 1-position

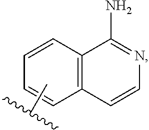

and optionally further substituted with 1 or 2 substituents as for heteroaryl^{a}.

When B is isoquinolinyl, B can be

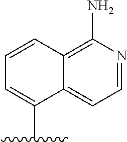 [SMILES: *c1cccc2c(N)nccc12]

optionally further substituted with 1 or 2 substituents as for heteroaryl^{a}.

Preferably, B is

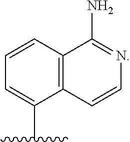 [SMILES: *c1cccc2c(N)nccc12]

Y can be as defined above. Preferably, Y is —N(R12)-. Preferably, Y is —NH— or —N(CH₃)—. Yet more preferably, Y is —NH—.

Preferably, B is heteroaryl^{a} and Y is —NH—.

Preferably, B is heteroaryl^{a} substituted with NH₂, and optionally substituted with 1 or 2 further substituents as for heteroaryl^{a}; and Y is —NH—.

Preferably, B is isoquinolinyl, optionally substituted as for heteroaryl^{a}; and Y is —NH—.

Preferably, B is

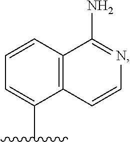 [SMILES: *c1cccc2c(N)nccc12]

### and Y is —NH—.

AW— can be as defined above. AW— can be selected from —(CH₂)₀₋₃—(CHR15)-(CH₂)₀₋₃-A, —(CHR12)-A, —O—(CHR12)-A, —(CH₂)₀₋₃-A, —(CH₂)₀₋₃—O—(CH₂)₀₋₃-A, —(CH₂)₀₋₃—NH—(CH₂)₀₋₃-A, —(CH₂)₀₋₃—N(R12)-(CH₂)₁₋₃—C(═O)-A, —(CH₂)₀₋₃—NH—C(═O)—(CH₂)₀₋₃-A, —C(═O)N(R12)-(CH₂)₀₋₃-A, —(CH₂)₀₋₃—C(═O)—(CH₂)₀₋₃-A, —(CH₂)₀₋₃-(phenyl)-(CH₂)₀₋₃-A, —NH—SO₂-A and —SO₂—NH-A.

AW— can be selected from —(CH₂)₀₋₃-A, and —(CH₂)₀₋₃—O—(CH₂)₀₋₃-A.

AW— can be selected from —(CH₂)₀₋₁-A, and —(CH₂)₀₋₁—O-A.

Preferably, AW— is selected from —(CH₂)₀₋₁-A, and —(CH₂)—O-A.

Preferably, AW— is selected from —(CH₂)₀₋₁-A, and —(CH₂)—O-A; and Y is —NH—.

Preferably, AW— is selected from —(CH₂)₀₋₁-A, and —(CH₂)—O-A; and B is heteroaryl^{a}.

Preferably, AW— is selected from —(CH₂)₀₋₁-A, and —(CH₂)—O-A; and B is heteroaryl^{a} substituted with NH₂, and optionally substituted with 1 or 2 further substituents as for heteroaryl^{a}.

Preferably, AW— is selected from —(CH₂)₀₋₁-A, and —(CH₂)—O-A; and B is isoquinolinyl, optionally substituted as for heteroaryl^{a}.

Preferably, AW— is selected from —(CH₂)₀₋₁-A, and —(CH₂)—O-A; and B is

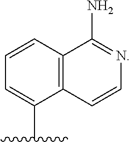 [SMILES: *c1cccc2c(N)nccc12]

Preferably, AW— is selected from —(CH₂)₀₋₁-A, and —(CH₂)—O-A; B is heteroaryl^{a}; and Y is —NH—.

Preferably, AW— is selected from —(CH₂)₀₋₁-A, and —(CH₂)—O-A; B is heteroaryl^{a} substituted with NH₂, and optionally substituted with 1 or 2 further substituents as for heteroaryl^{a}; and Y is —NH—.

Preferably, AW— is selected from —(CH₂)₀₋₁-A, and —(CH₂)—O-A; B is isoquinolinyl, optionally substituted as for heteroaryl^{a}; and Y is —NH—.

Preferably, AW— is selected from —(CH₂)₀₋₁-A, and —(CH₂)—O-A; B is

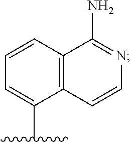 [SMILES: *c1cccc2c(N)nccc12]

### and Y is —NH—.

The invention provides compounds of formula (I), wherein
V is —N(R23)-, and Z is selected from —C(R16)(R17)-CH₂— and —C(R16)(R17)-; or,
V is selected from —CH₂—C(R16)(R17)- and —C(R16)(R17)-, and Z is —N(R18)-.

More specifically, the invention provides compounds of formula (I) wherein the compound is a compound of formula (Ia) or formula (Ib),

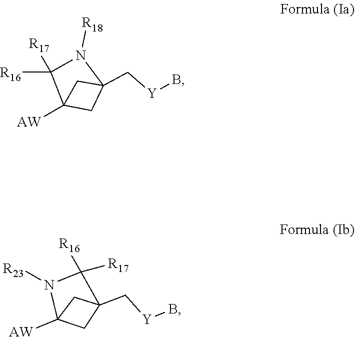

or a tautomer, isomer, stereoisomer (including an enantiomer, a diastereoisomer and a racemic and scalemic mixture thereof), a deuterated analogue, and a pharmaceutically acceptable salt and/or solvate thereof.

R16 can be H. R16 and R17 can both be H.

Preferably, R16 and R17 are both H; and B is heteroaryl^{a}.

Preferably, R16 and R17 are both H; and B is heteroaryl^{a} substituted with NH₂, and optionally substituted with 1 or 2 further substituents as for heteroaryl^{a}.

Preferably, R16 and R17 are both H; and B is isoquinolinyl, optionally substituted as for heteroaryl^{a}.

Preferably, R16 and R17 are both H; and B is

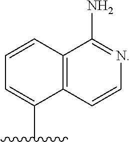 [SMILES: *c1cccc2c(N)nccc12]

Preferably, R16 and R17 are both H; and Y is —NH—.

Preferably, R16 and R17 are both H; B is heteroaryl^{a}; and Y is —NH—.

Preferably, R16 and R17 are both H; B is heteroaryl^{a} substituted with NH₂, and optionally substituted with 1 or 2 further substituents as for heteroaryl^{a}; and Y is —NH—.

Preferably, R16 and R17 are both H; B is isoquinolinyl, optionally substituted as for heteroaryl^{a}; and Y is —NH—.

Preferably, R16 and R17 are both H; B is

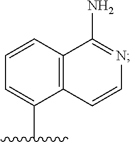 [SMILES: *c1cccc2c(N)nccc12]

### and Y is —NH—.

R18 or R23 can be selected from C(═S)NH-aryl; C(═S)NH—R19; C(═S)N(alkyl)R19; C(═S)NR13R14; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one substituent selected from cycloalkyl and heterocycloalkyl^{b}, and optionally one or two further substituents independently selected from alkyl, alkoxy, OH, OCF₃, halo, CN, and CF₃;

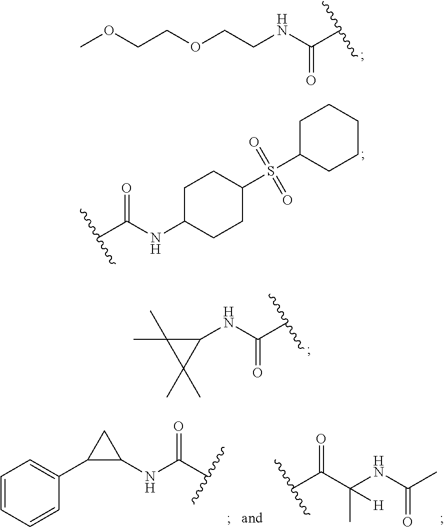

wherein R13 and R14 together with the nitrogen atom to which they are attached form a carbon-containing 4-, 5-, 6- or 7-membered heterocyclic ring, optionally containing an additional heteroatom selected from N, N(R12), S, SO, SO₂, and O, which may be saturated or unsaturated with 1 or 2 double bonds and which may be optionally mono- or di-substituted with substituents selected from oxo, alkyl^{b}, alkoxy, OH, halo and CF₃.

R18 or R23 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

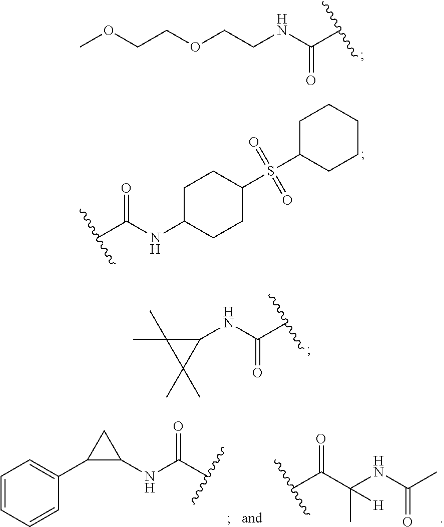

R18 or R23 can be selected from C(═S)NH-phenyl; C(═O)R19; C(═O)NHR19; C(═O)N(alkyl)(R19); C(═O)NH₂, heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

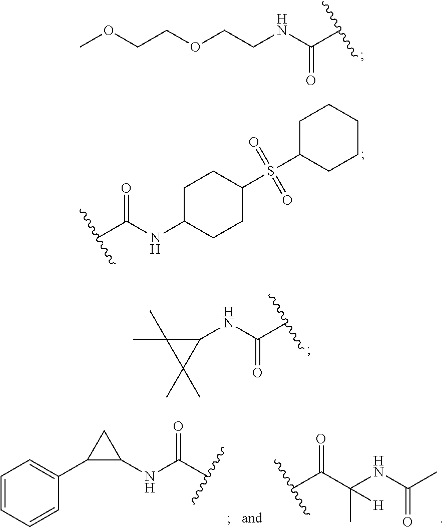

Preferably, R18 or R23 can be selected from C(═S)NH-phenyl; C(═O)R19; C(═O)NHR19; C(═O)N(alkyl)(R19); C(═O)NH₂; heterocycloalkyl^{b}; a 6-membered carbon-containing aromatic ring containing one or two N ring members substituted with one cycloalkyl substituent;

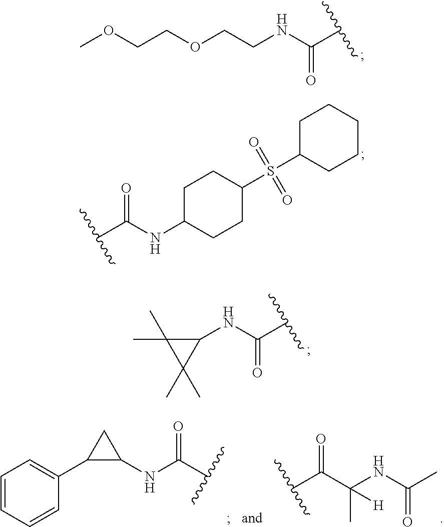

Yet more preferably, R18 or R23 can be selected from C(═S)NH-phenyl; C(═O)R19; C(═O)NHR19; C(═O)N(alkyl)(R19); C(═O)NH₂; 5- or 6-membered heterocycloalkyl^{b} ring, optionally substituted as for heterocycloalkyl^{b}; a 6-membered carbon-containing aromatic ring containing one or two N ring members substituted with one cycloalkyl substituent;

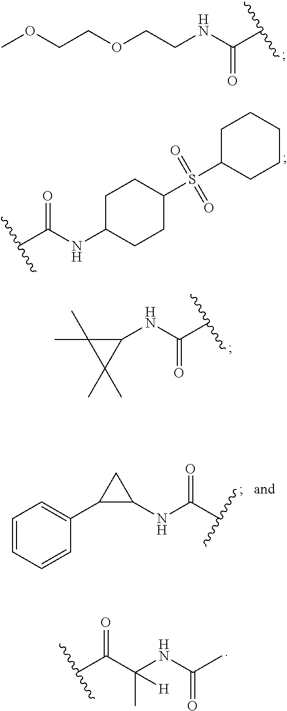

Yet more preferably, R18 or R23 can be selected from C(═S)NH-phenyl; C(═O)R19; C(═O)NHR19; C(═O)N(alkyl)(R19); C(═O)NH₂; 5-membered heterocycloalkyl^{b} ring, optionally substituted as for heterocycloalkyl^{b}; a 6-membered carbon-containing aromatic ring containing one or two N ring members substituted with one cycloalkyl substituent;

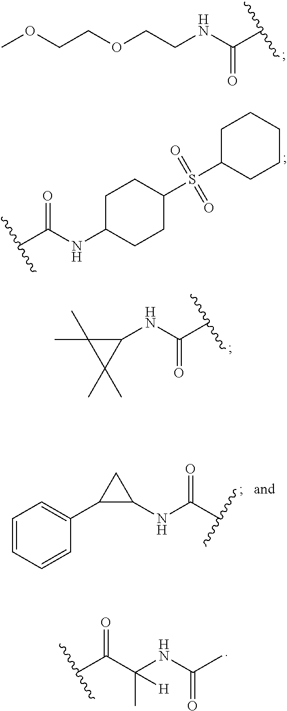

Yet more preferably, R18 or R23 can be selected from C(═S)NH-phenyl; C(═O)R19; C(═O)NHR19; C(═O)N(alkyl)(R19); C(═O)NH₂; 5-membered heterocycloalkyl^{b} ring, optionally substituted as for heterocycloalkyl^{b}; a 6-membered carbon-containing aromatic ring containing one or two N ring members substituted with one

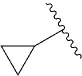 [SMILES: *C1CC1]

substituent;

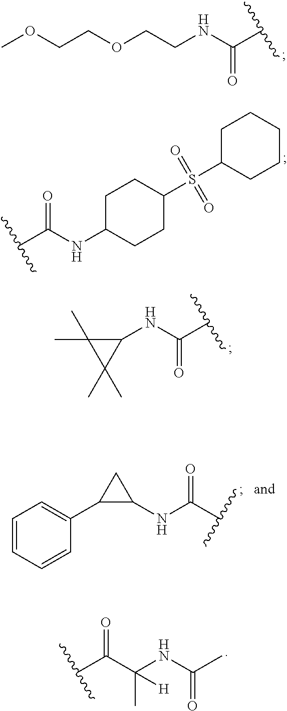

Yet more preferably, R18 or R23 can be selected from C(═S)NH-phenyl; C(═O)R19; C(═O)NHR19; C(═O)N(alkyl)(R19); C(═O)NH₂; 5-membered heterocycloalkyl^{b} ring, optionally substituted as for heterocycloalkyl^{b}; a 6-membered carbon-containing aromatic ring containing two N ring members substituted with one

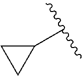 [SMILES: *C1CC1]

substituent;

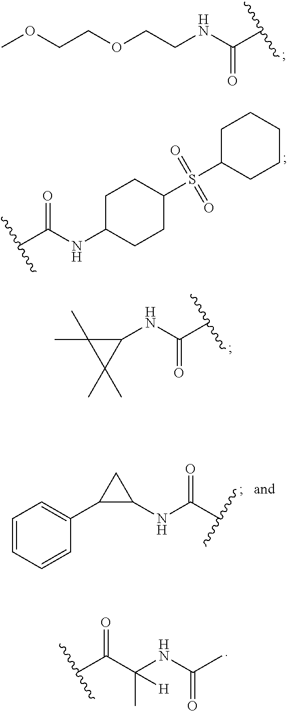

R18 can be selected from:

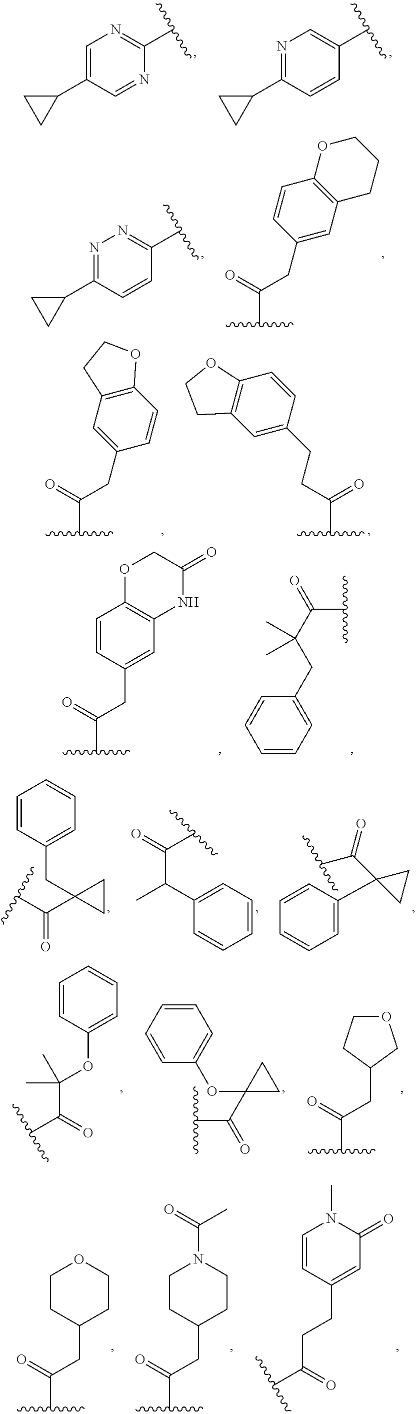

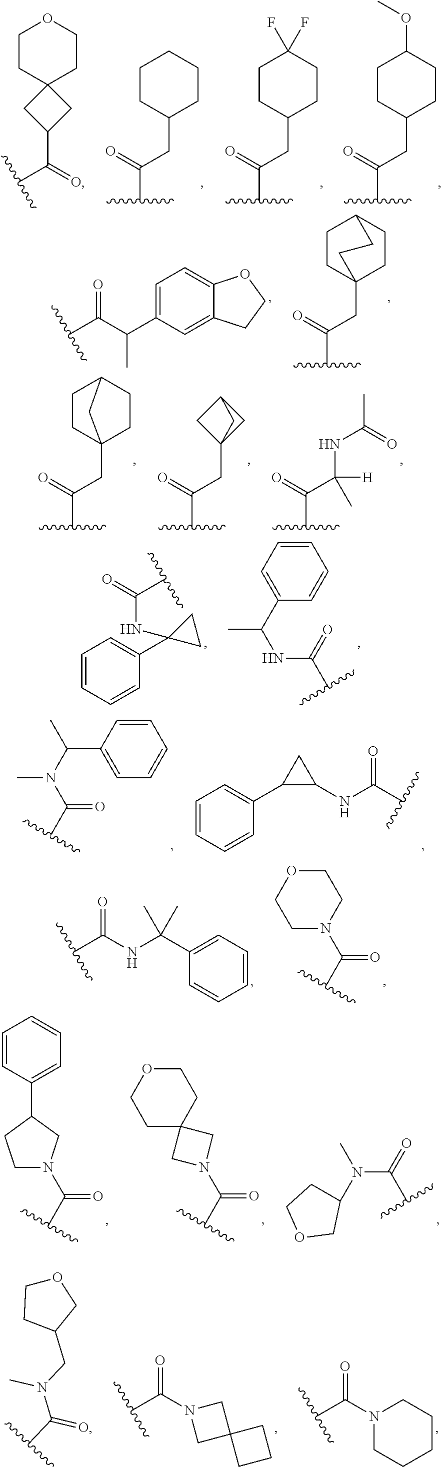

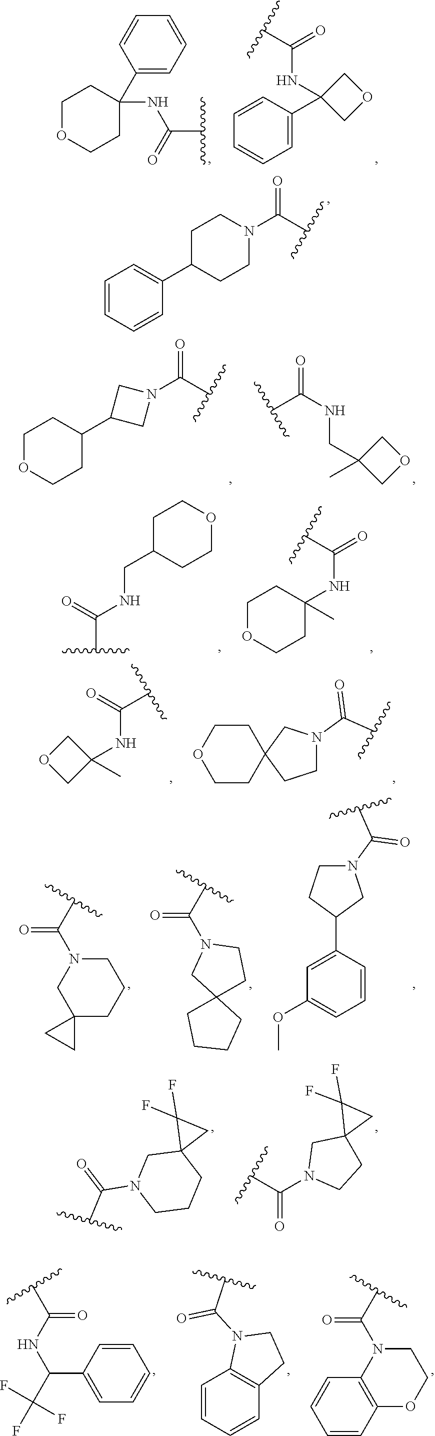

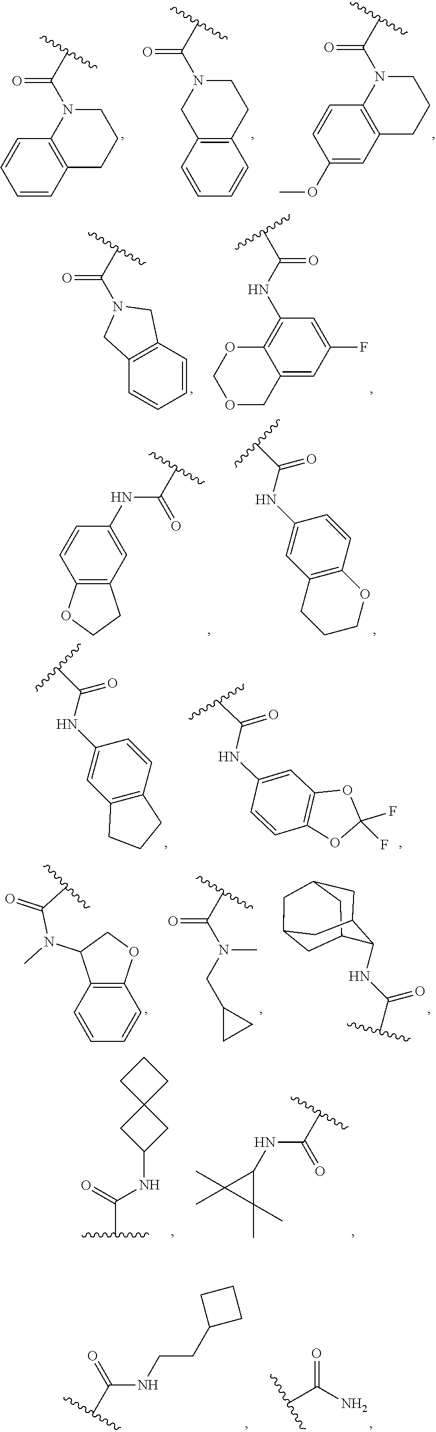

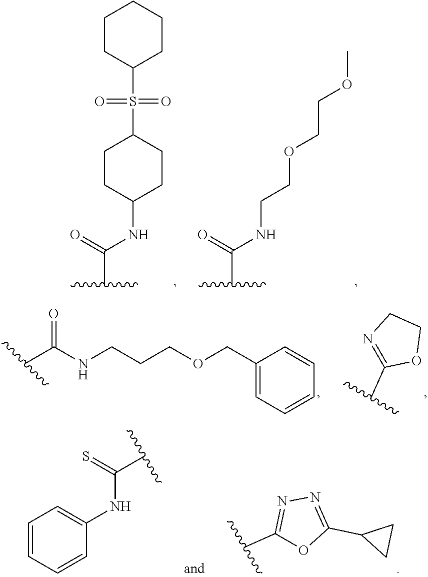 [SMILES: *C(=O)NC1CCC(S(=O)(=O)C2CCCCC2)CC1 ; *C(=O)NCCOCCOC ; *C(=O)NCCCOCc1ccccc1 ; *C1=NCCO1 ; *C(=S)Nc1ccccc1 ; *c1nnc(C2CC2)o1]

R18 can be selected from:

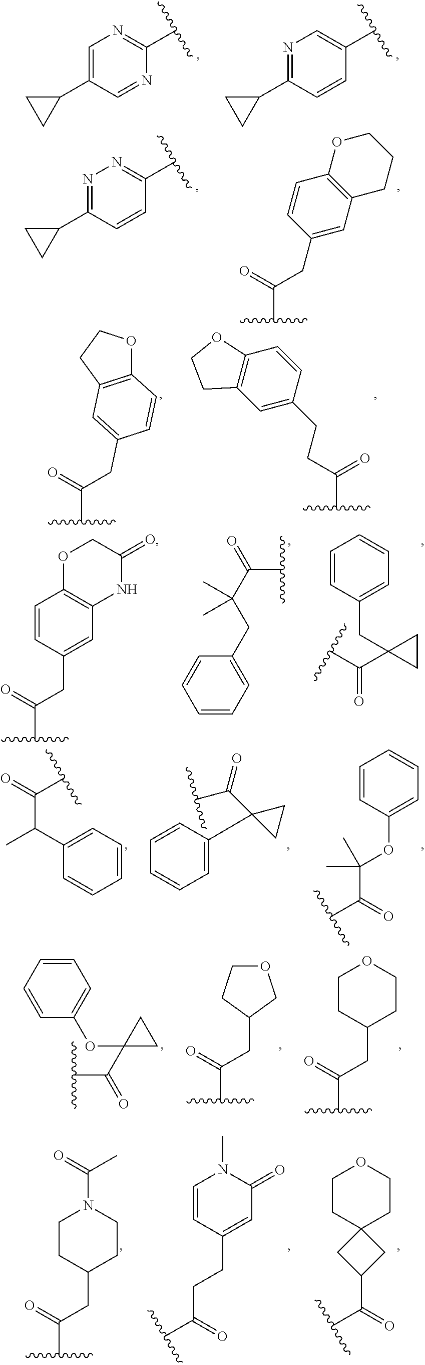

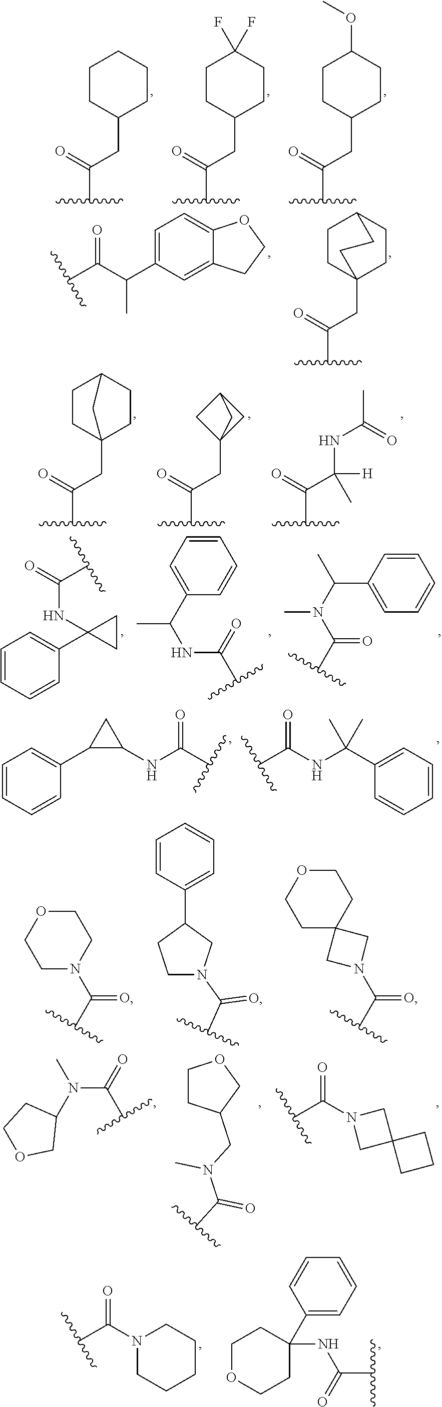

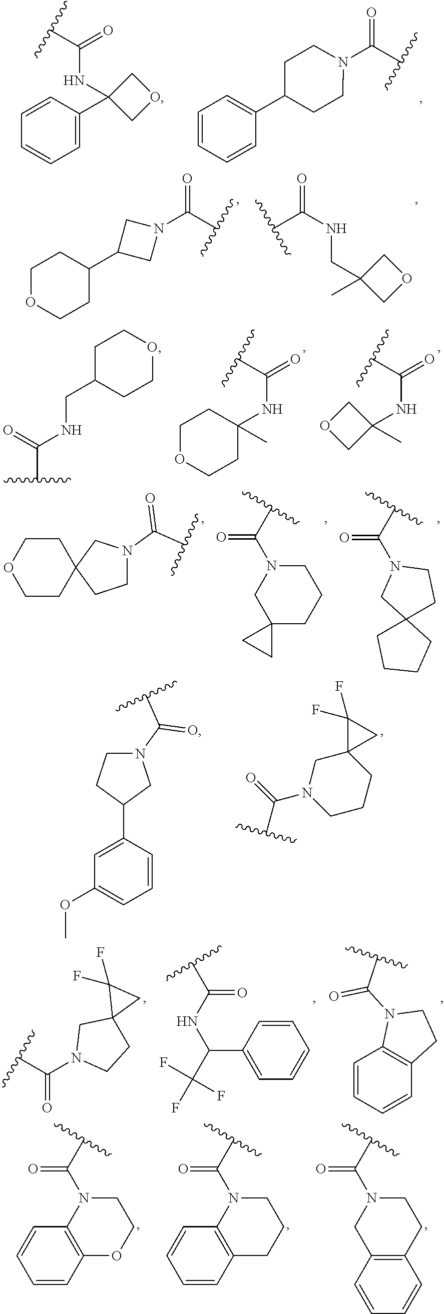

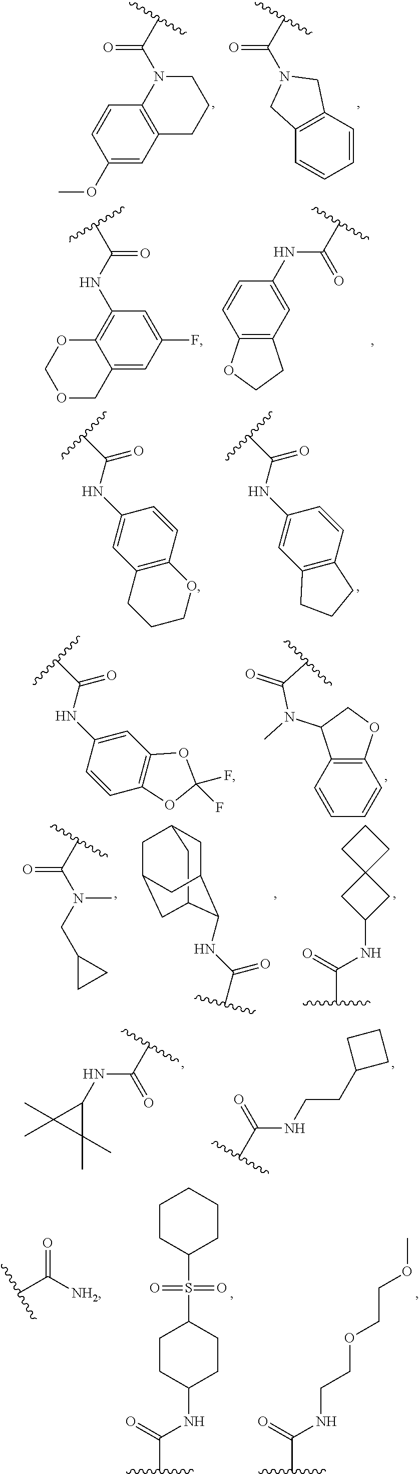

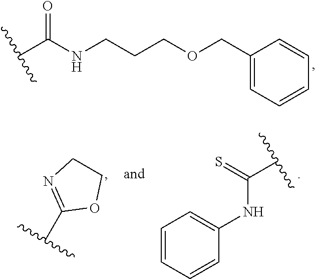 [SMILES: *C(=O)NCCCOCc1ccccc1 ; *C1=NCCO1 ; *C(=S)Nc1ccccc1]

R18 can be selected from:

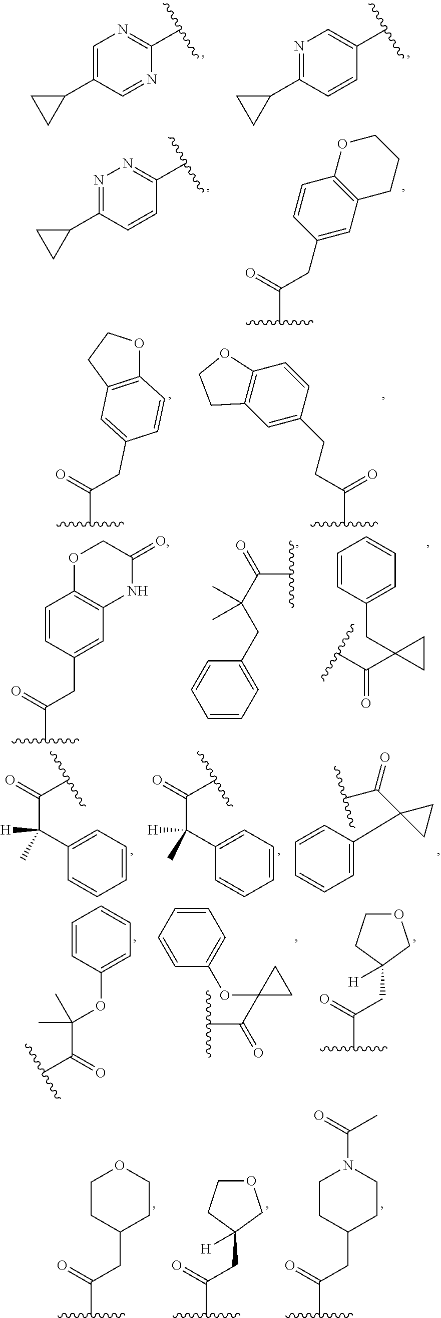

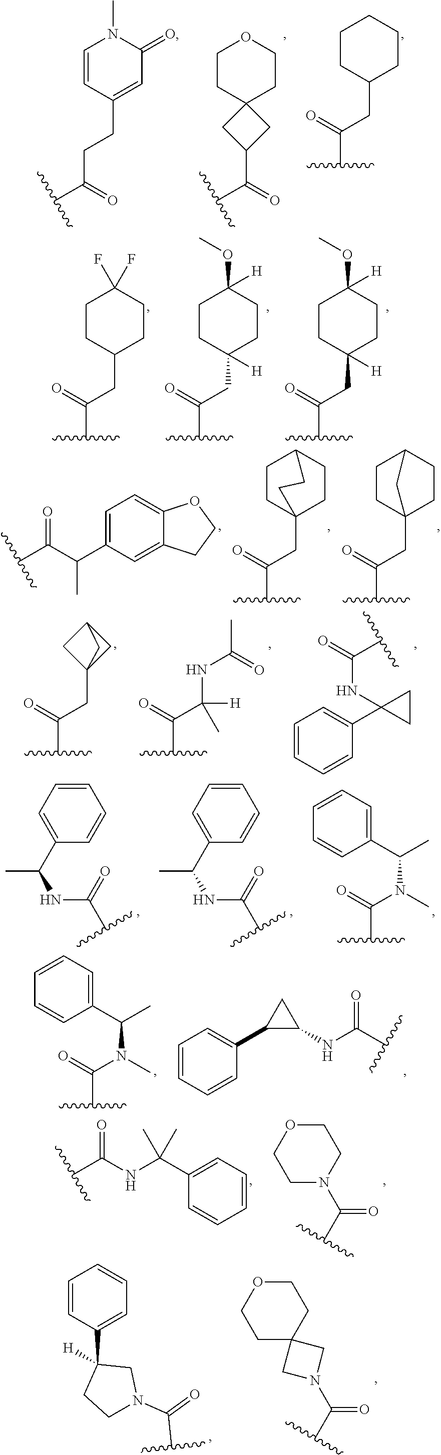

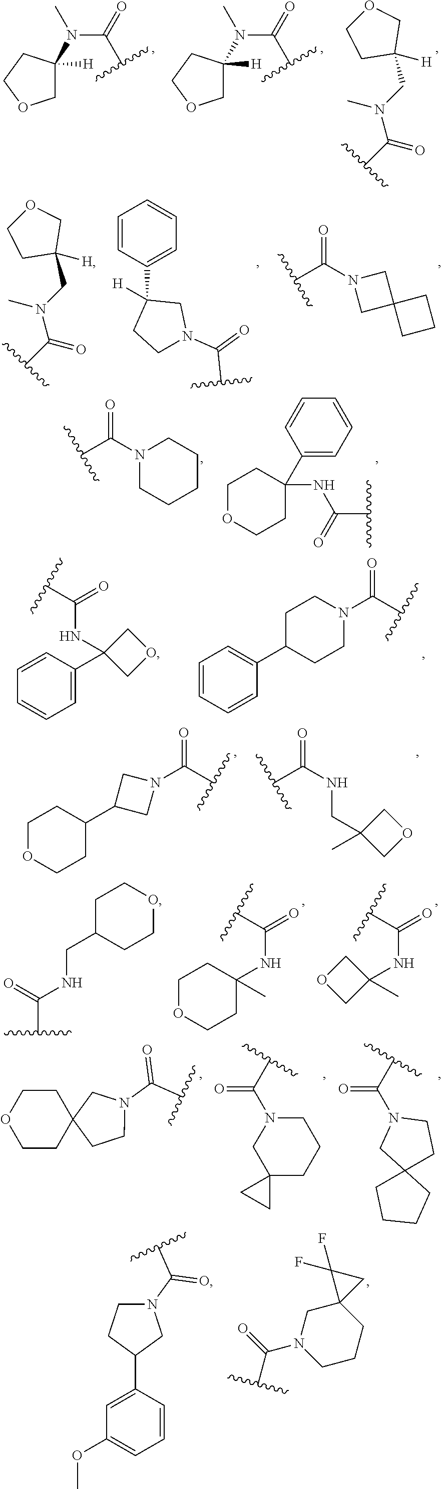

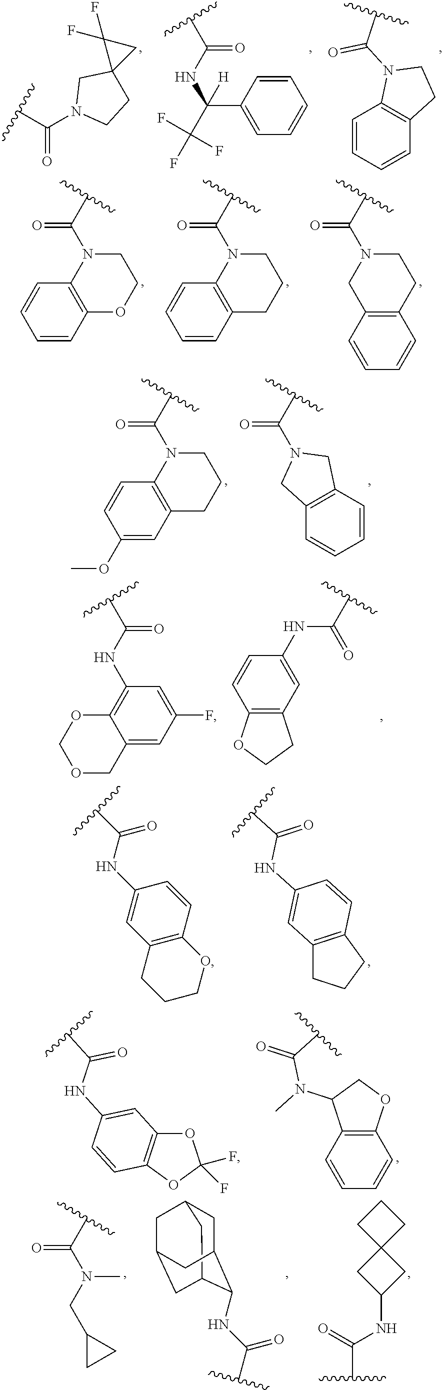

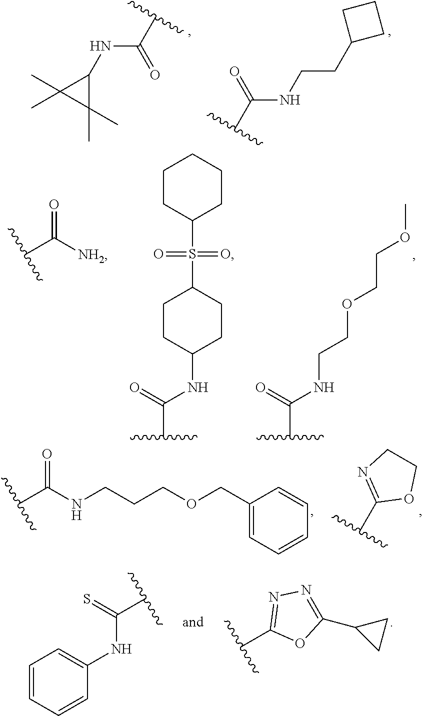

R18 can be selected from:

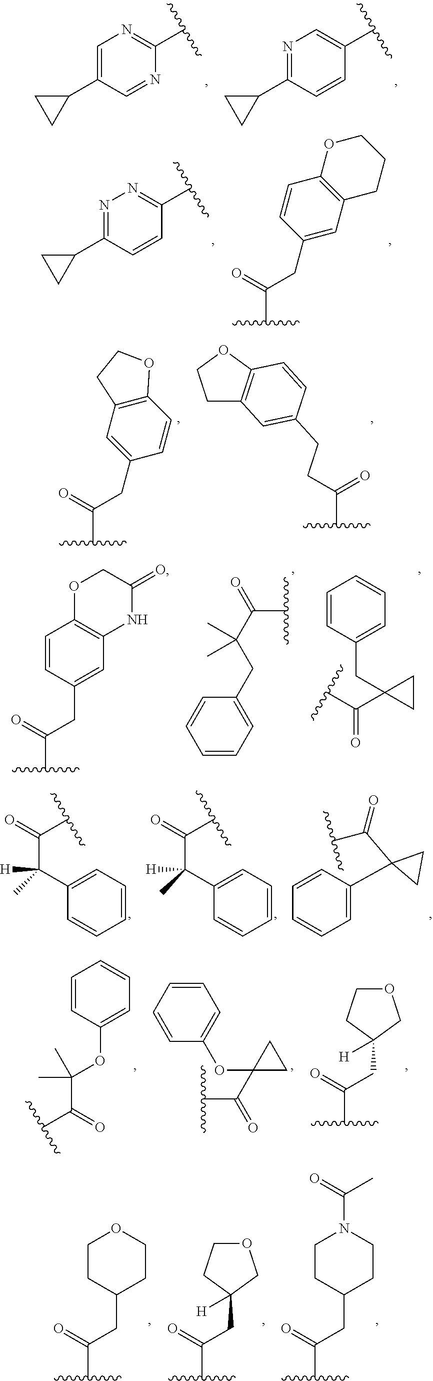

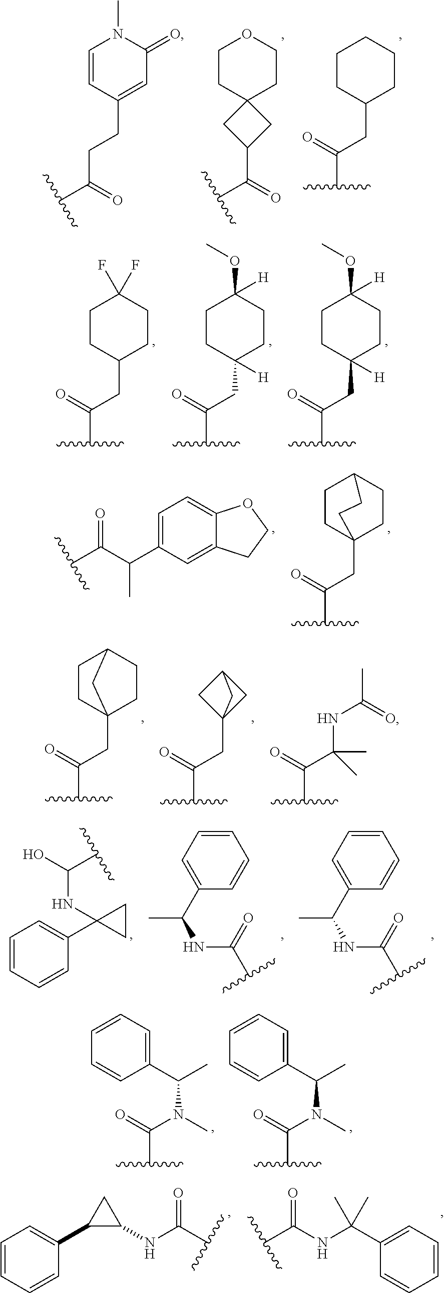

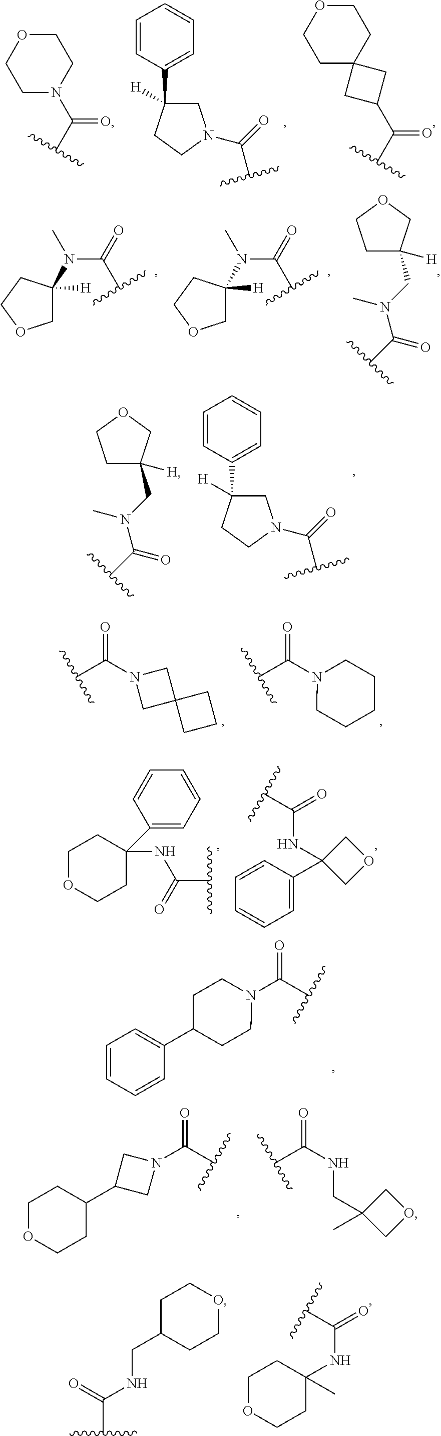

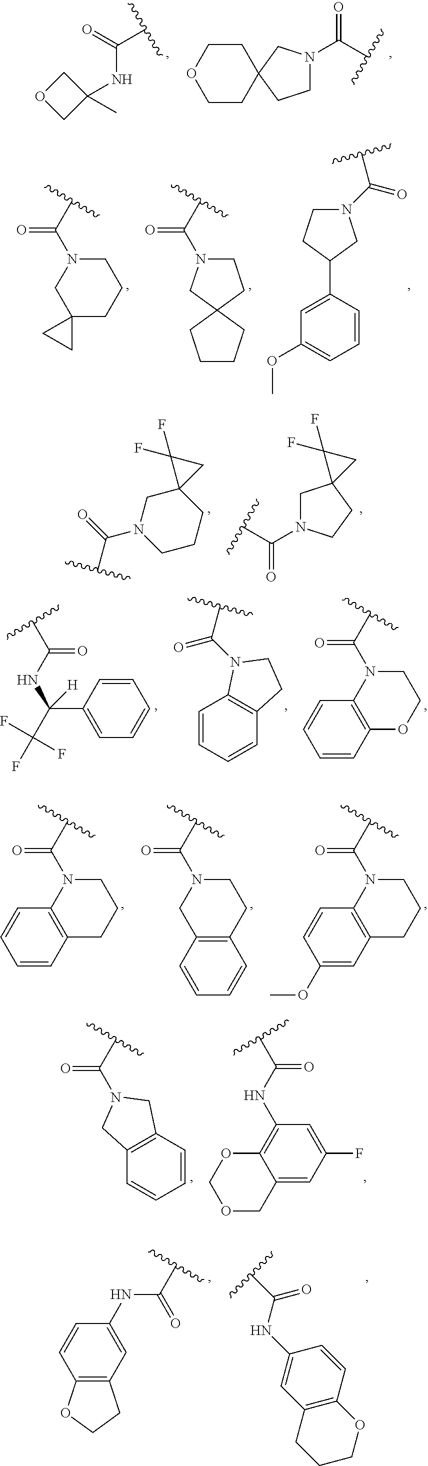

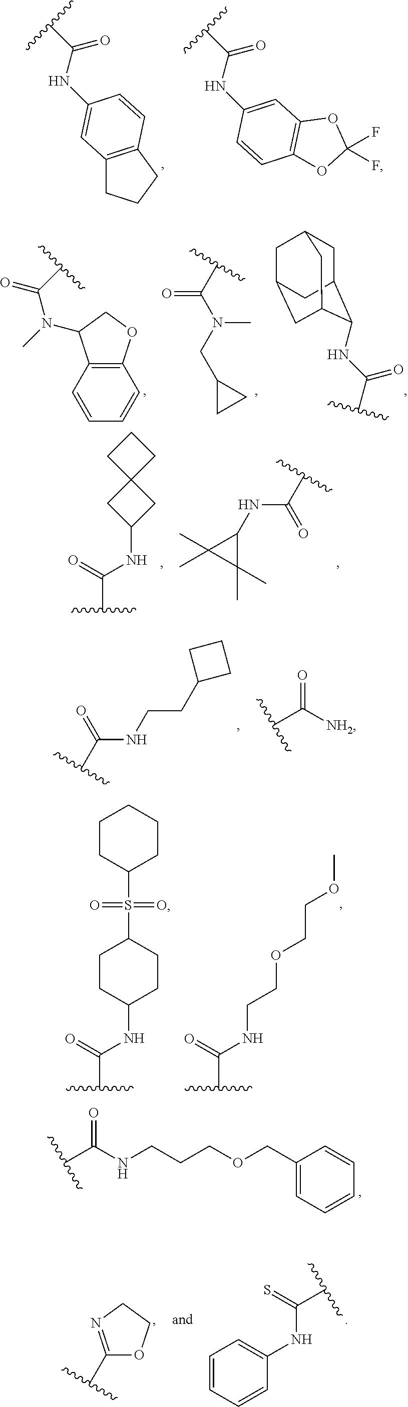

Preferably, R18 is selected from:

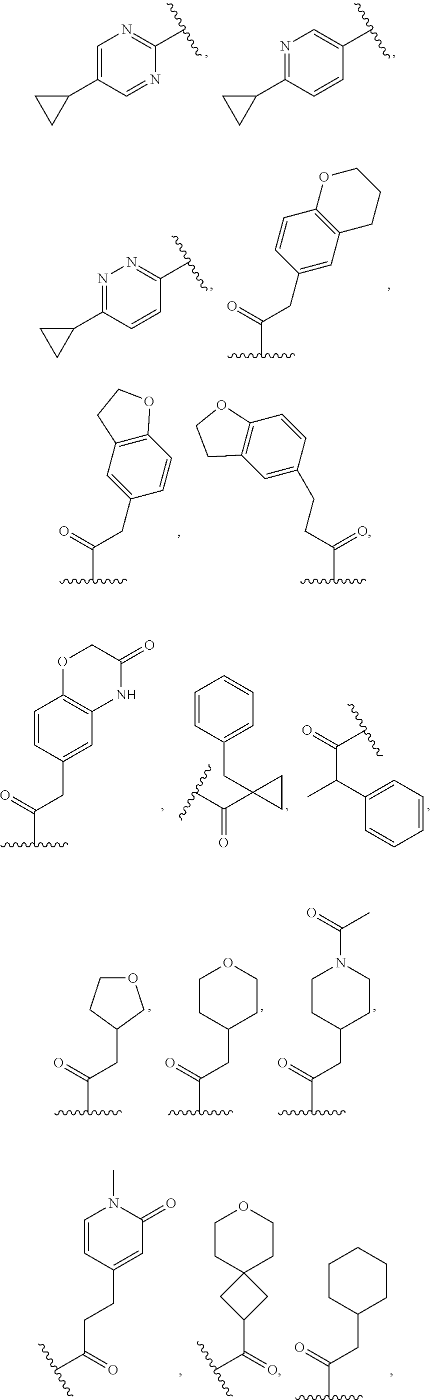

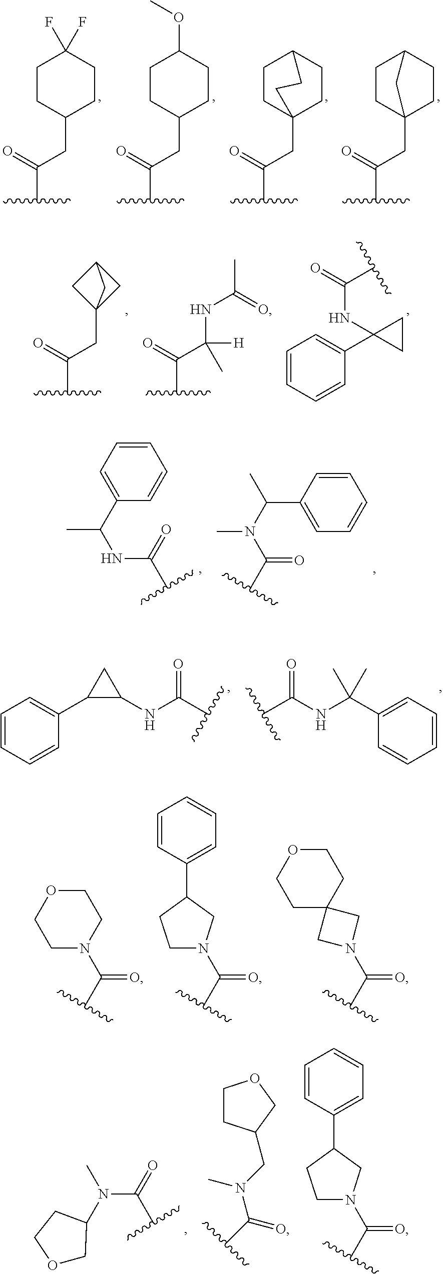

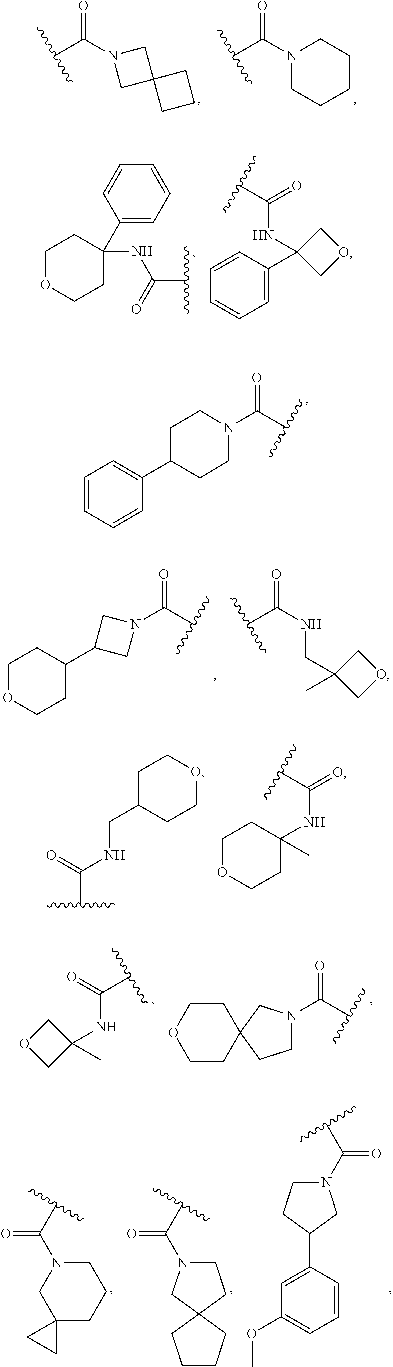

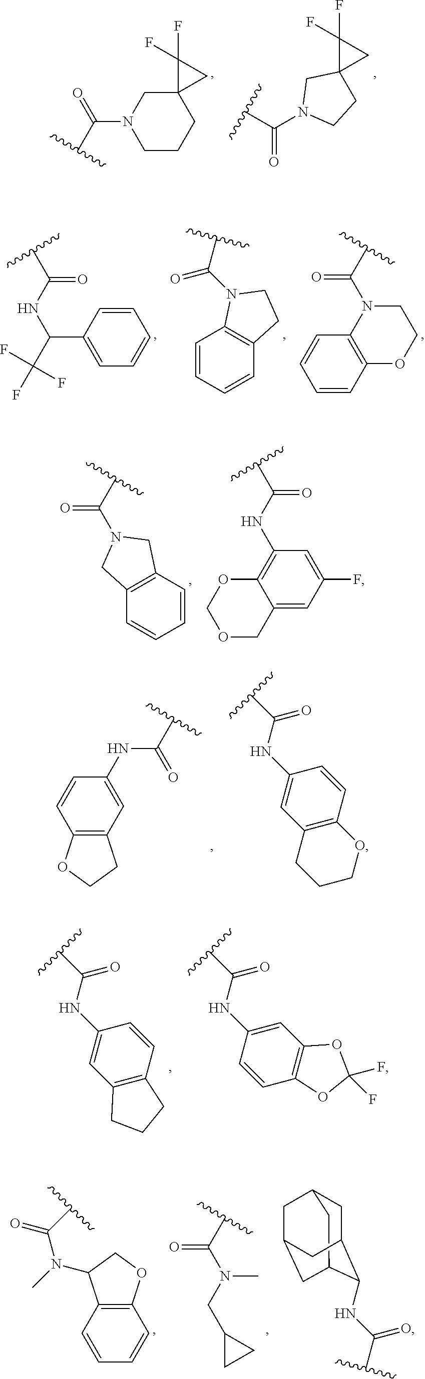

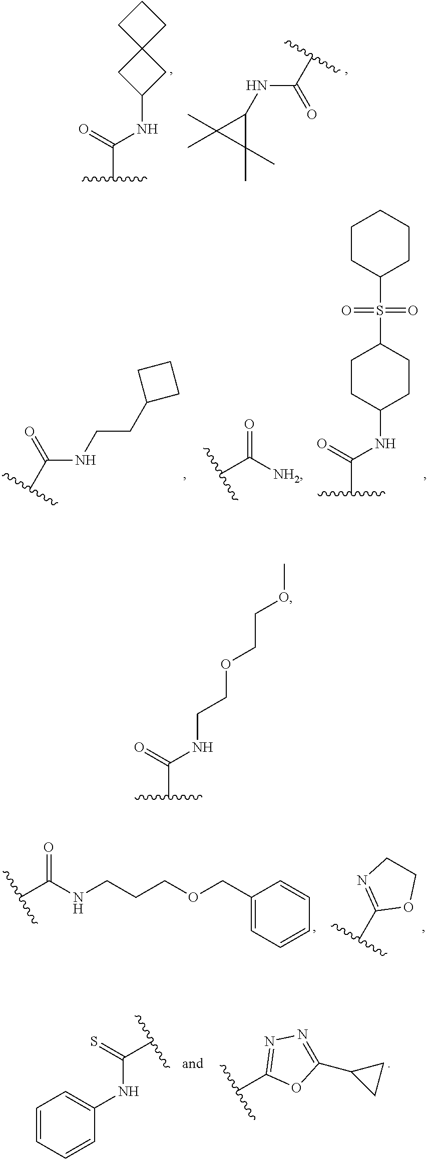

Preferably, R18 is selected from:

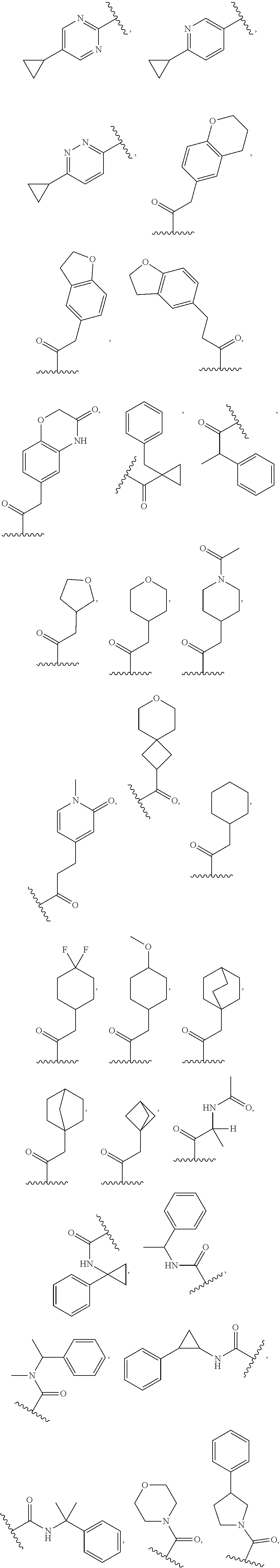

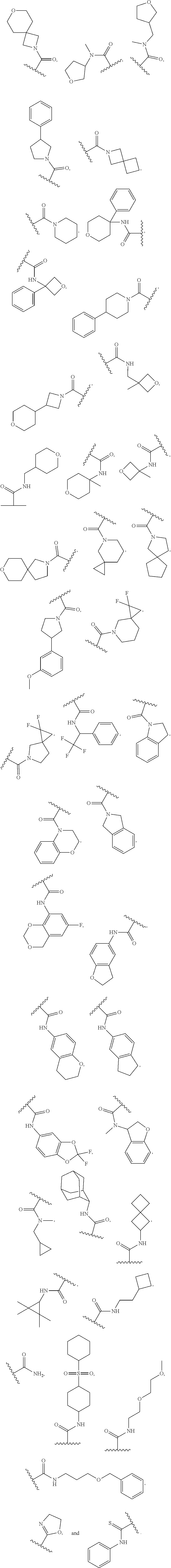

Preferably, R18 is selected from:

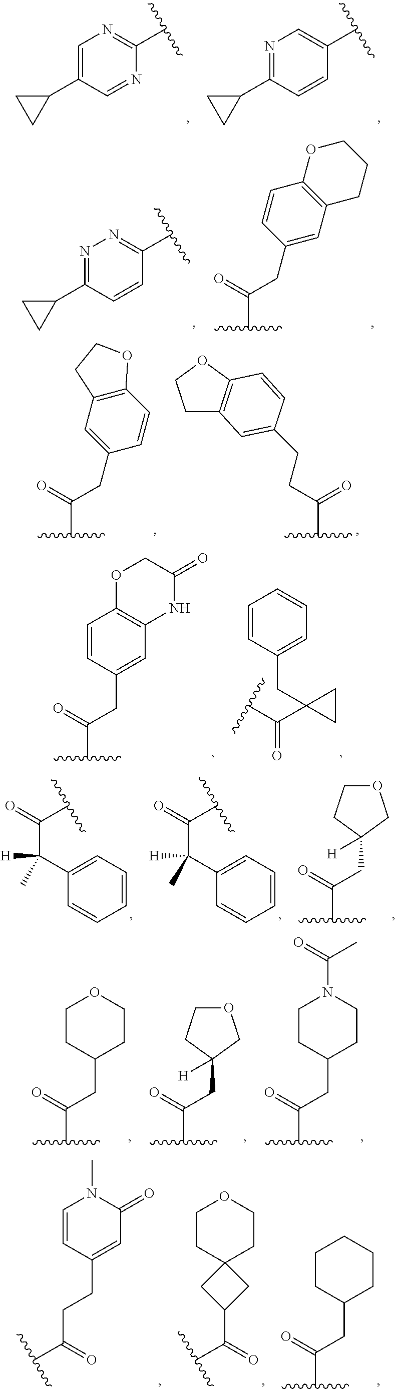

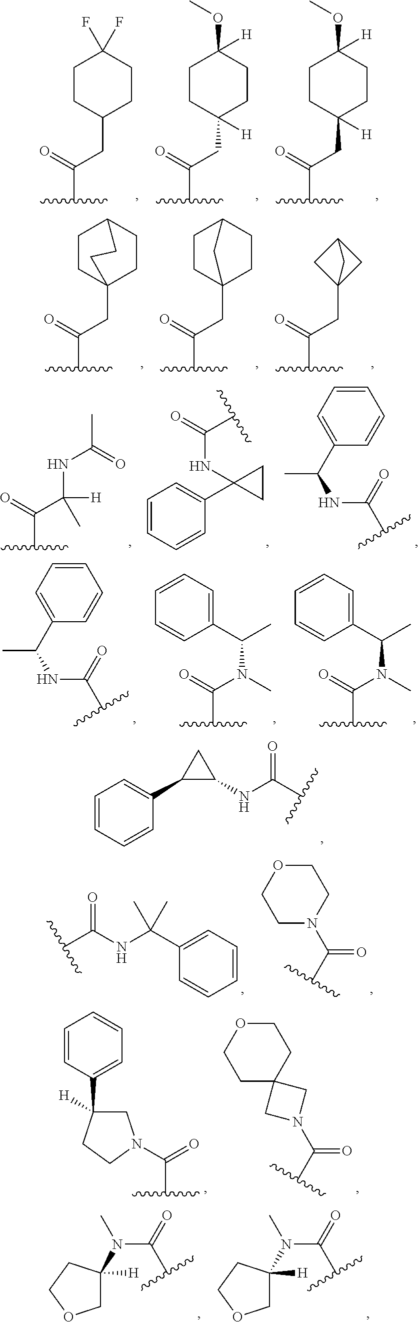

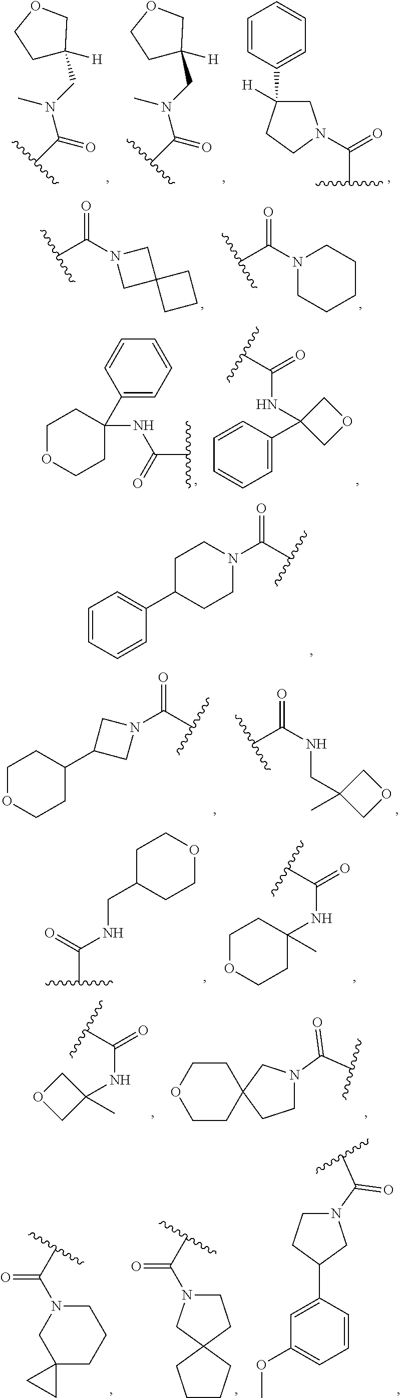

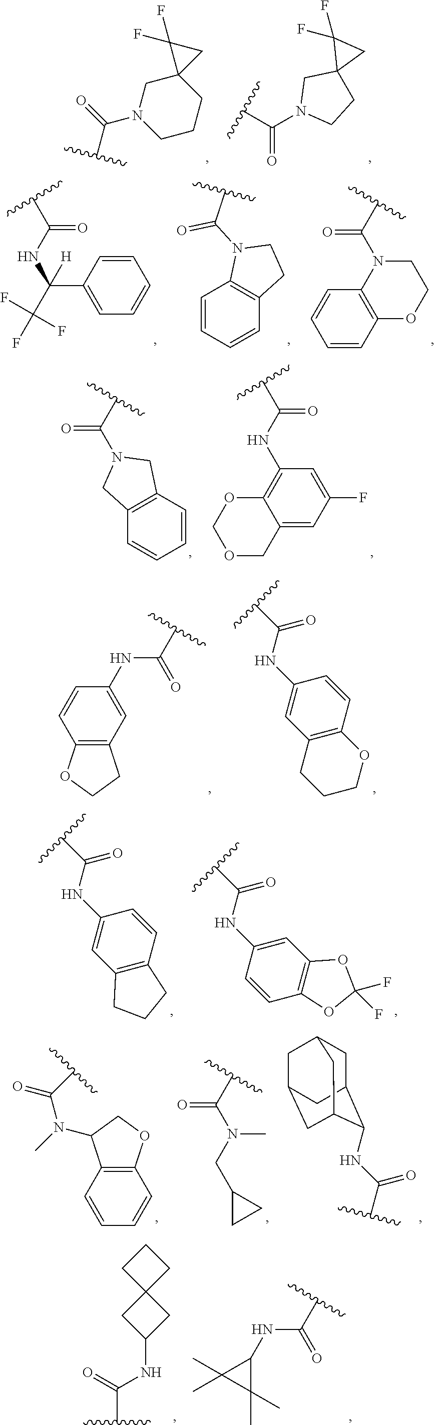

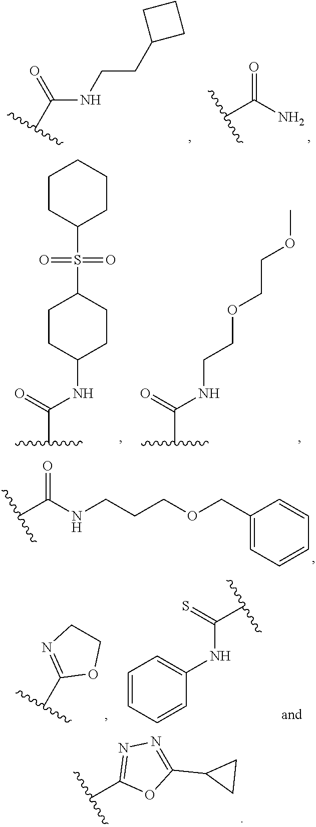

Preferably, R18 is selected from:

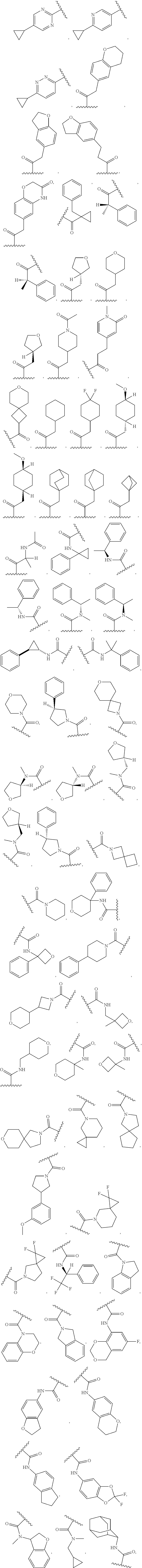

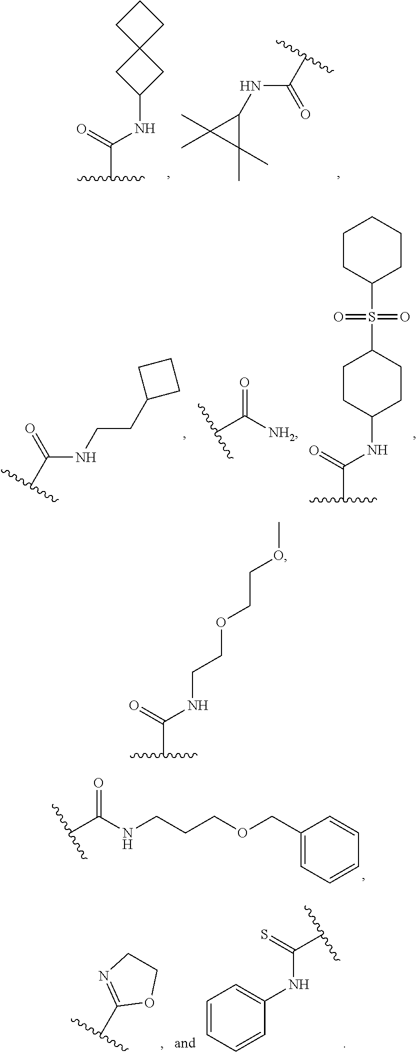

Yet more preferably, R18 is selected from:

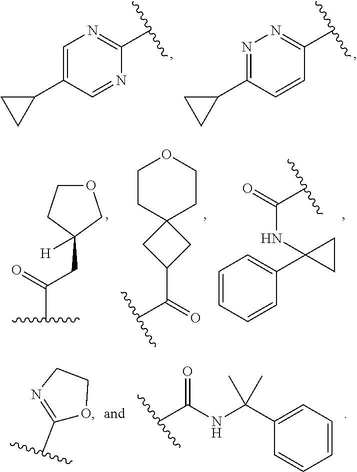

The compound of the invention can be a compound of formula (Ia), wherein R18 is as described as above, or a compound of formula (Ib) and R23 is C(═O)R19.

The compound of the invention can be a compound of formula (Ia), wherein R18 is as described as above, or a compound of formula (Ib) and R23 is C(═O)(CH₂)₁₋₃cycloalkyl.

The compound of the invention can be a compound of formula (Ia), wherein R18 is as described above, or a compound of formula (Ib) and R23 is C(═O)(CH₂)₁₋₂cycloalkyl.

The compound of the invention can be a compound of formula (Ia), wherein R18 is as described above, or a compound of formula (Ib) and R23 is selected from

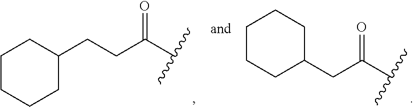 [SMILES: *C(=O)CCC1CCCCC1 ; *C(=O)CC1CCCCC1]

Preferably, R16 and R17 are both H; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

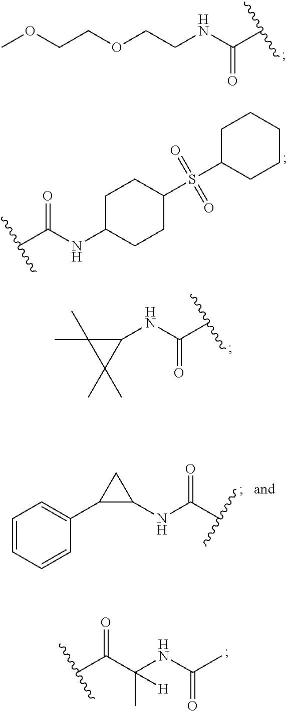

or a compound of formula (Ib) and R23 is C(═O)(CH₂)₁₋₂cycloalkyl.

Preferably, Y is —NH—; and the compound of the invention is a compound formula (Ia), wherein R18 can be selected from C(═S)NH-aryl; C(═O)R19; C(═O)NH R19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

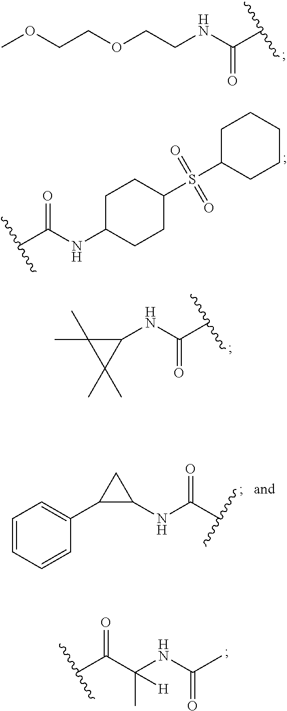

or a compound of formula (Ib) and R23 is C(═O)(CH₂)₁₋₂cycloalkyl.

Preferably, B is heteroaryl^{a}; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

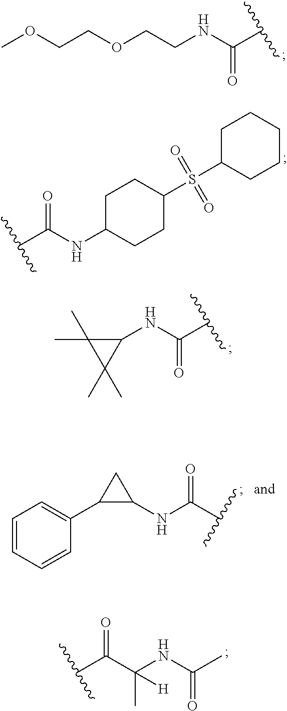

or a compound of formula (Ib) and R23 is C(═O)(CH₂)₁₋₂cycloalkyl.

Preferably, B is heteroaryl^{a} substituted with NH₂, and optionally substituted with 1 or 2 further substituents as for heteroaryl^{a}; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

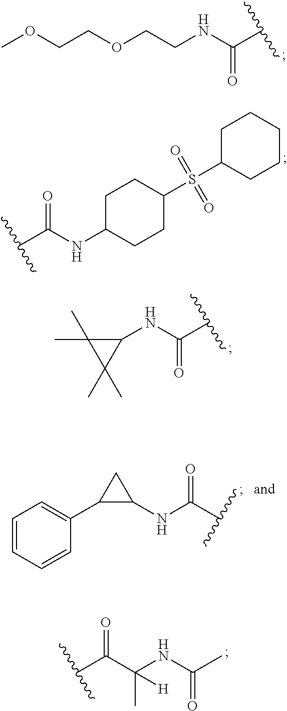

or a compound of formula (Ib) and R23 is C(═O)(CH₂)₁₋₂cycloalkyl.

Preferably, B is isoquinolinyl, optionally substituted as for heteroaryl^{a}; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

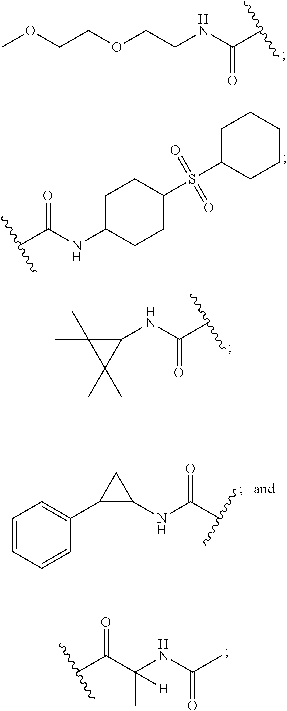

or a compound of formula (Ib) and R23 is C(═O)(CH₂)₁₋₂cycloalkyl.

Preferably, B is

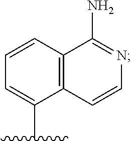 [SMILES: *c1cccc2c(N)nccc12]

and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

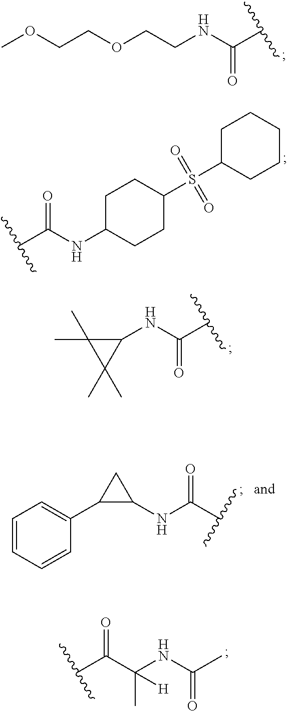

or a compound of formula (Ib) and R23 is C(═O)(CH₂)₁₋₂cycloalkyl.

Preferably, B is heteroaryl^{a}; Y is —NH—; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

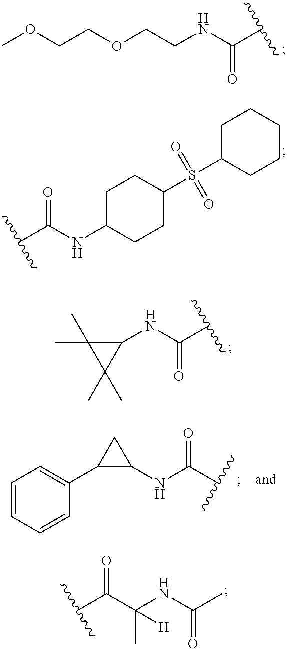

or a compound of formula (Ib) and R23 is C(═O)(CH₂)₁₋₂cycloalkyl.

Preferably, B is heteroaryl^{a} substituted with NH₂, and optionally substituted with 1 or 2 further substituents as for heteroaryl^{a}; Y is —NH—; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

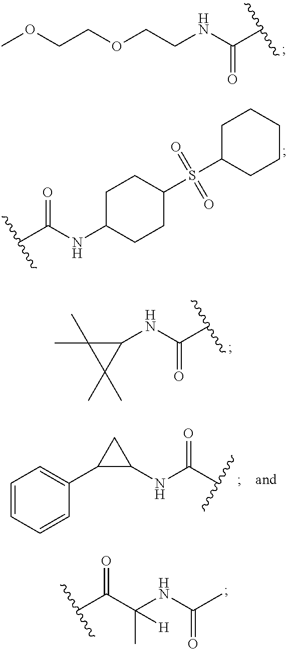

or a compound of formula (Ib) and R23 is C(═O)(CH₂)₁₋₂cycloalkyl.

Preferably, B is isoquinolinyl, optionally substituted as for heteroaryl^{a}; Y is —NH—; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

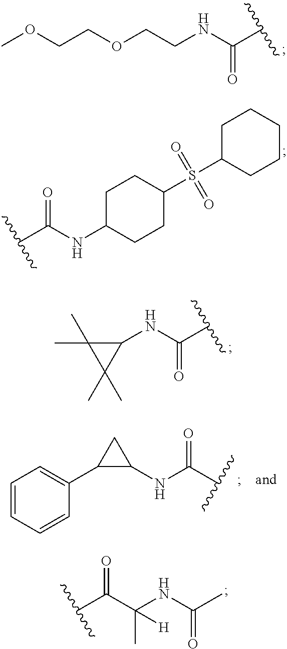

or a compound of formula (Ib) and R23 is C(═O)(CH₂)₁₋₂cycloalkyl.

preferably, B is

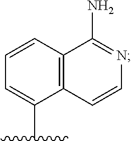 [SMILES: *c1cccc2c(N)nccc12]

Y is —NH—; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

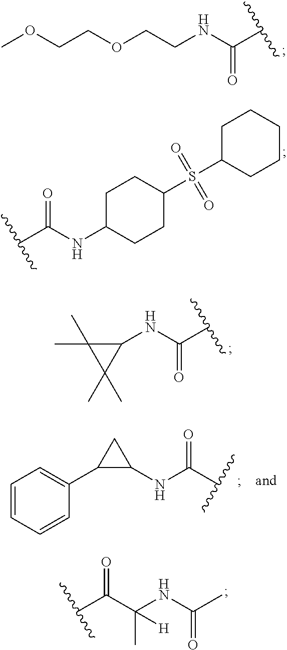

or a compound of formula (Ib) and R23 is C(═O)(CH₂)₁-cycloalkyl.

Preferably, R16 and R17 are both H; B is heteroaryl^{a}; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

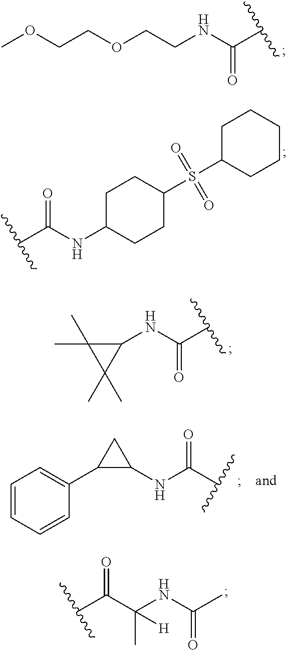

or a compound of formula (Ib) and R23 is C(═O)(CH₂)₁₋₂cycloalkyl.

Preferably, R16 and R17 are both H; B is heteroaryl^{a} substituted with NH₂, and optionally substituted with 1 or 2 further substituents as for heteroaryl^{a}; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

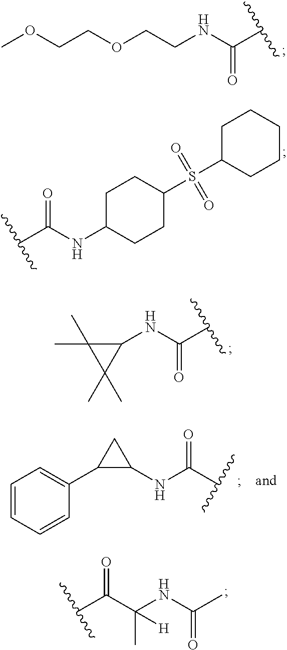

or a compound of formula (Ib) and R23 is C(═O)(CH₂)₁₋₂cycloalkyl.

Preferably, R16 and R17 are both H; B is isoquinolinyl, optionally substituted as for heteroaryl^{a}; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

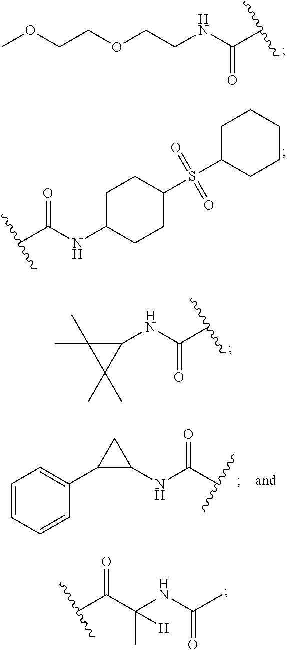

or a compound of formula (Ib) and R23 is C(═O)(CH₂)₁₋₂cycloalkyl.

Preferably, R16 and R17 are both H; B is

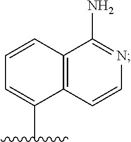 [SMILES: *c1cccc2c(N)nccc12]

and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

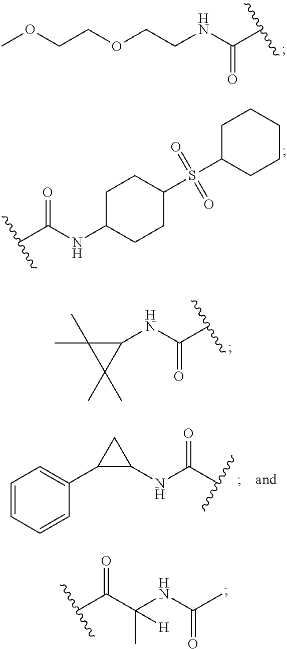

or a compound of formula (Ib) and R23 is C(═O)(CH₂)₁₋₂cycloalkyl.

Preferably, R16 and R17 are both H; Y is —NH—; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

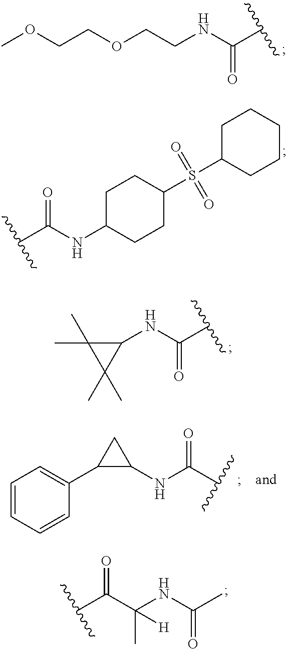

or a compound of formula (Ib) and R23 is C(═O)(CH₂)₁₋₂cylcloalkyl.

Preferably, R16 and R17 are both H; B is heteroaryl^{a}; Y is —NH—; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

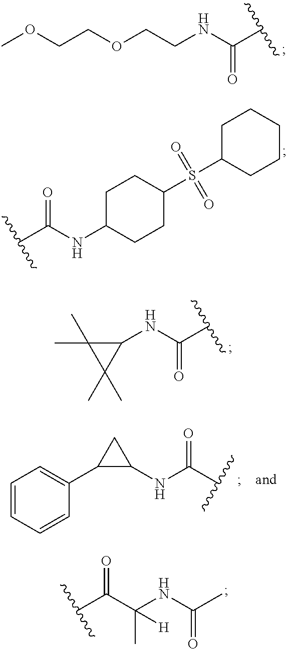

or a compound of formula (Ib) and R23 is C(═O)(CH₂)₁₋₂cycloalkyl.

Preferably, R16 and R17 are both H; B is heteroaryl^{a} substituted with NH₂, and optionally substituted with 1 or 2 further substituents as for heteroaryl^{a}; Y is —NH—; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

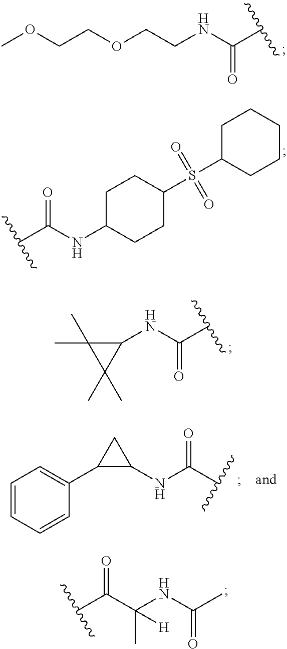

or a compound of formula (Ib) and R23 is C(═O)(CH₂)₁₋₂cycloalkyl.

Preferably, R16 and R17 are both H; B is isoquinolinyl, optionally substituted as for heteroaryl^{a}; Y is —NH—; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent:

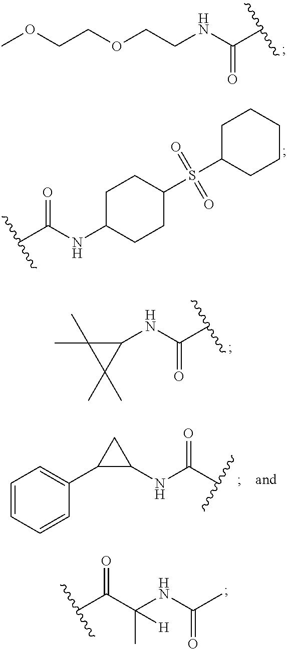

or a compound of formula (Ib) and R23 is C(═O)(CH₂)₁₋₂cycloalkyl.

Preferably, R16 and R17 are both H; B is

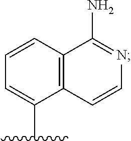 [SMILES: *c1cccc2c(N)nccc12]

Y is —NH—; and the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

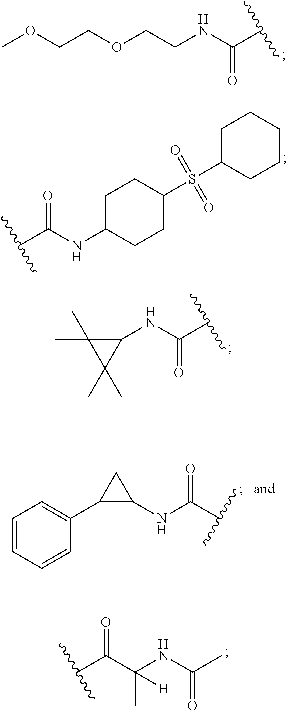

or a compound of formula (Ib) and R23 is C(═O)(CH₂)₁₋₂cycloalkyl.

R19 can be selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R19 can be selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, and (CH₂)₃—O—(CH₂)-phenyl.

Heterocycloalkyl^{a} can be a 4-10 membered non-aromatic carbon-containing mono- or bi-cyclic ring system containing one or two ring members independently selected from N and O; heterocycloalkyl^{a} may optionally be substituted with 1 or 2 substituents independently selected from alkyl, aryl, halo, and heterocycloalkyl; wherein when heterocycloalkyl^{a} is a bicyclic ring system, the bicyclic ring system is spiro.

Heterocycloalkyl^{a} can be selected from:

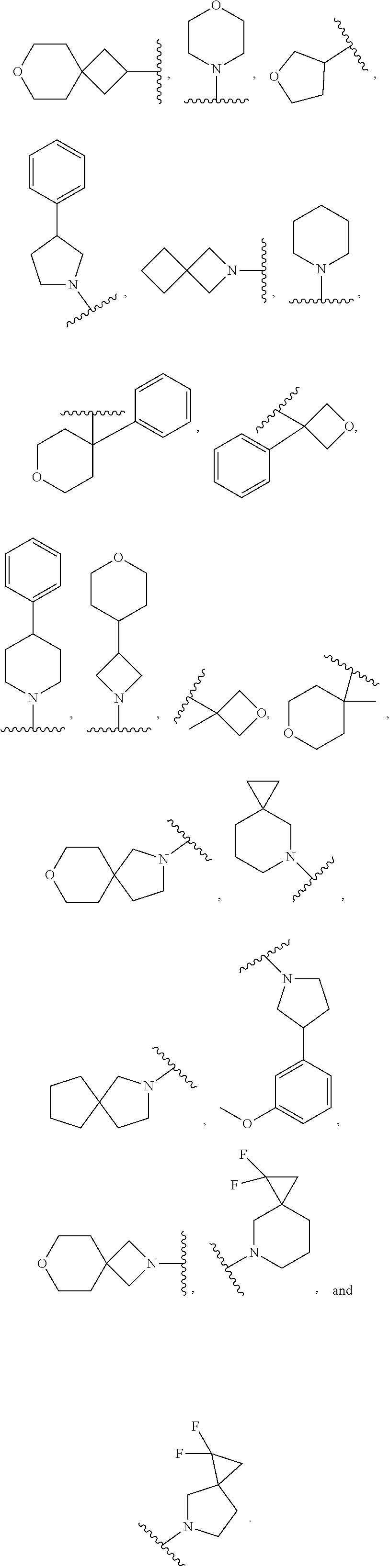

Heterocycloalkyl^{a} can be selected from:

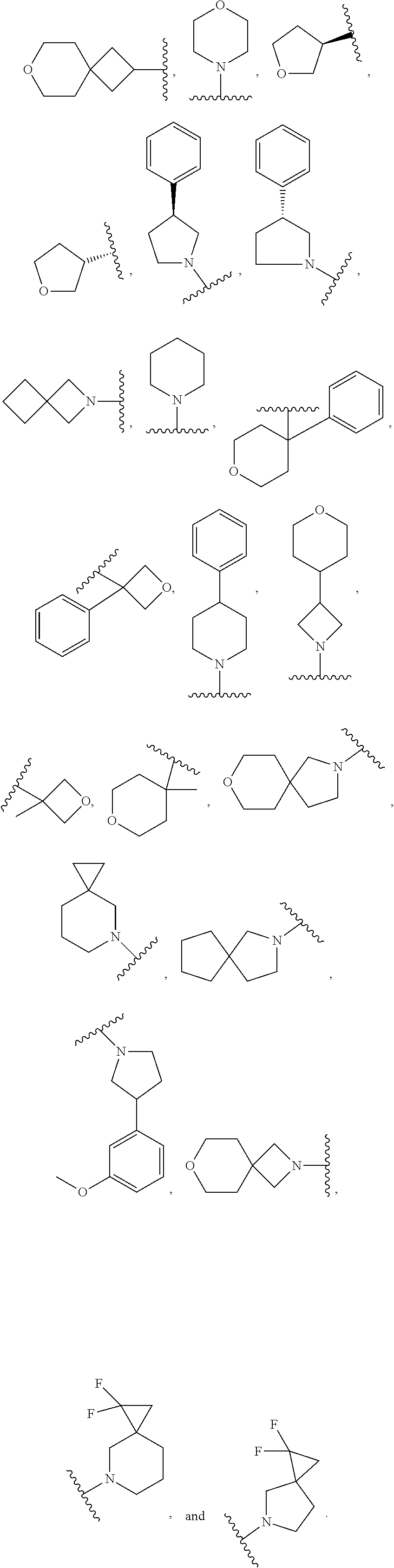

Heterocycloalkyl^{b} can be a 4-6 membered non-aromatic carbon-containing monocyclic ring system which may be saturated or, where possible, unsaturated with 1 or 2 double bonds; heterocycloalkyl^{b} contains one or two ring members independently selected from N, N(C(═O)CH₃) and O; heterocycloalkyl^{b} may optionally be substituted with 1 or 2 substituents independently selected from alkyl, phenyl and oxo; wherein the point of attachment of heterocycloalkyl^{b} is not at a ring nitrogen.

Heterocycloalkyl^{b} can be selected from:

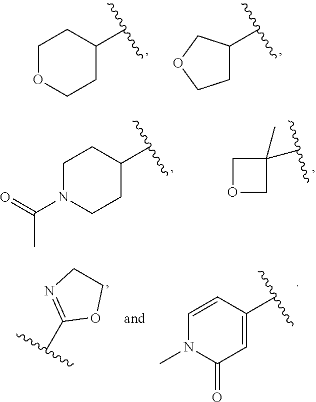

Heterocycloalkyl^{b} can be selected from:

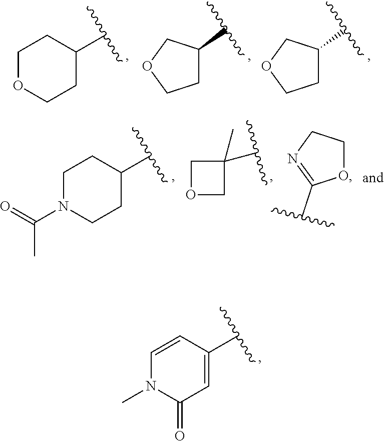

Polycycloalkyl can be selected from;

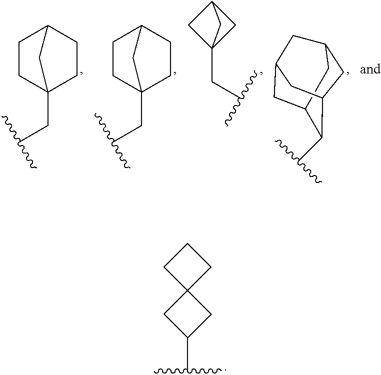

R22 can be a fused 6,5- or 6,6-bicyclic ring containing an aromatic ring fused to a non-aromatic ring, wherein the bicyclic ring optionally contains one or two ring members selected from N and O, wherein the fused 6,5- or 6,6-bicyclic ring may be optionally substituted with one or two substituents independently selected from alkoxy, halo, and oxo, wherein the 6,5-bicyclic ring may be attached via the 6- or 5-membered ring, wherein the 6,5-bicyclic ring is not aryl substituted with methylenedioxy, and wherein the 6,6-bicyclic ring is not aryl substituted with ethylenedioxy.

R22 can be a fused 6,5- or 6,6-bicyclic ring containing a 6-membered aromatic ring fused to a non-aromatic ring, wherein the bicyclic ring optionally contains one or two ring members selected from N and O, wherein the fused 6,5- or 6,6-bicyclic ring may be optionally substituted with one or two substituents independently selected from alkoxy, halo, and oxo, wherein the 6,5-bicyclic ring may be attached via the 6- or 5-membered ring, wherein the 6,5-bicyclic ring is not aryl substituted with methylenedioxy, and wherein the 6,6-bicyclic ring is not aryl substituted with ethylenedioxy.

R22 can be selected from:

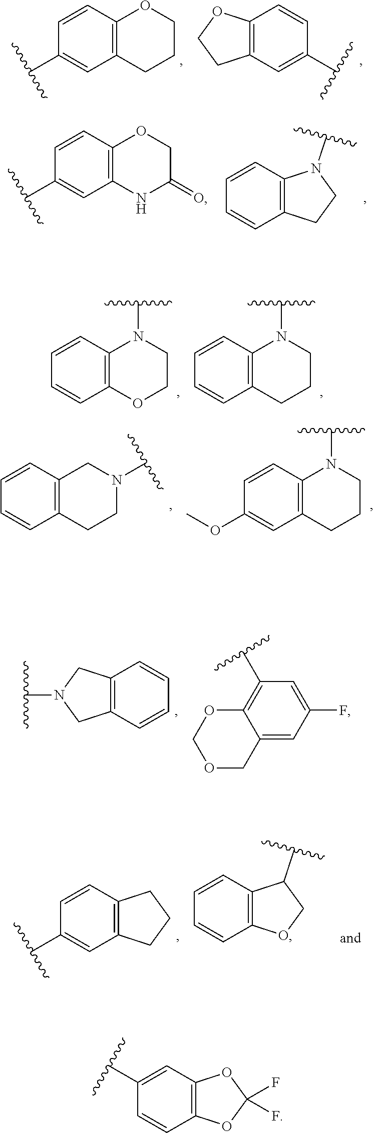

Preferably R22 is selected from:

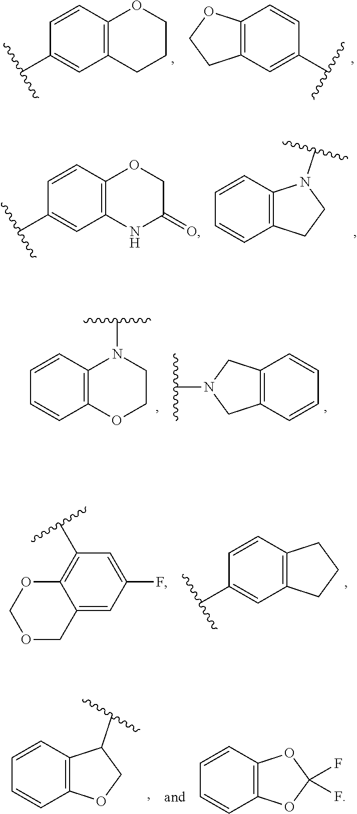

R20 and R21 together with the carbon atom to which they are attached can form a carbon-containing 3-, 4- or 6-membered saturated ring, optionally containing an O ring member, wherein R20 and R21 are not both H.

Alternatively, R20 or R21 can be independently selected from H, (C₁₋₃)alkyl and CF₃, wherein R20 and R21 are not both H.

More specifically, R20 or R21 can be independently selected from H, methyl and CF₃, wherein R20 and R21 are not both H.

Preferably, R16 and R17 are both H; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, Y is —NH—; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is heteroaryl^{a}; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is heteroaryl^{a} substituted with NH₂, and optionally substituted with 1 or 2 further substituents as for heteroaryl^{a}; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is isoquinolinyl, optionally substituted as for heteroaryl^{a}; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is

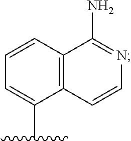 [SMILES: *c1cccc2c(N)nccc12]

and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

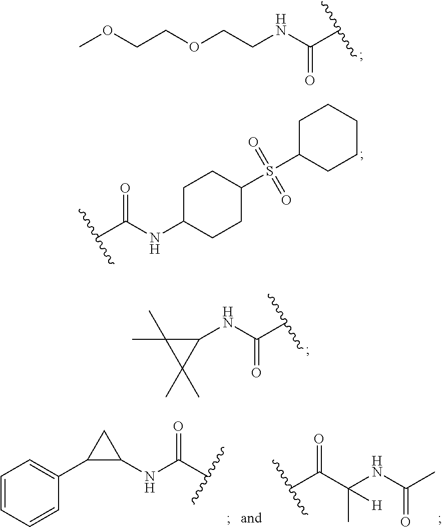

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is heteroaryl^{a}; Y is —NH—; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is heteroaryl^{a} substituted with NH₂, and optionally substituted with 1 or 2 further substituents as for heteroaryl^{a}; Y is —NH—; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is isoquinolinyl, optionally substituted as for heteroaryl^{a}; Y is —NH—; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

preferably, B is

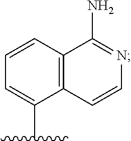 [SMILES: *c1cccc2c(N)nccc12]

Y is —NH—; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is heteroaryl^{a}; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is heteroaryl^{a} substituted with NH₂, and optionally substituted with 1 or 2 further substituents as for heteroaryl^{a}; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is isoquinolinyl, optionally substituted as for heteroaryl^{a}; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is

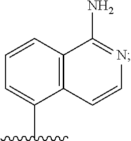 [SMILES: *c1cccc2c(N)nccc12]

and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; Y is —NH—; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is heteroaryl^{a}; Y is —NH—; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is heteroaryl^{a} substituted with NH₂, and optionally substituted with 1 or 2 further substituents as for heteroaryl^{a}; Y is —NH—; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is isoquinolinyl, optionally substituted as for heteroaryl^{a}; Y is —NH—; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is

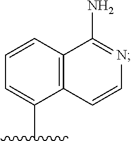 [SMILES: *c1cccc2c(N)nccc12]

Y is —NH—; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

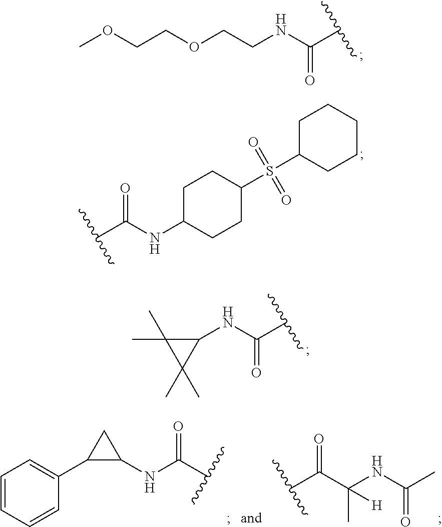

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, Y is —NH—; the compound of the invention is a compound formula (Ia), wherein R18 can be selected from C(═S)NH-aryl; C(═O)R19; C(═O)NH R19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

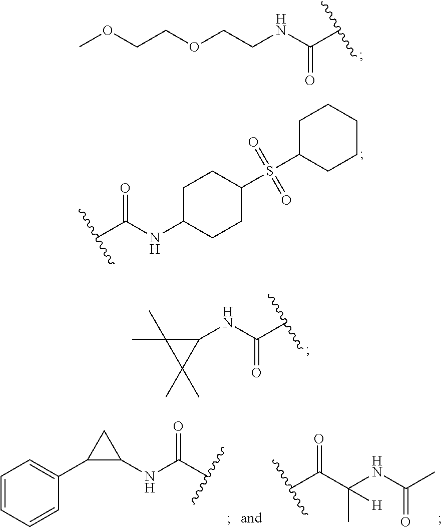

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is heteroaryl^{a}; the compound of the invention is a compound formula (Ia), wherein R18 can be selected from C(═S)NH-aryl; C(═))R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

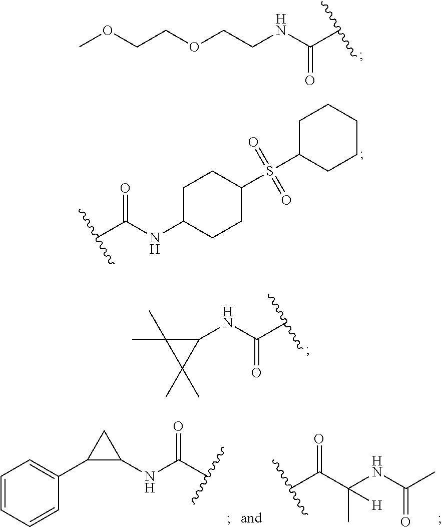

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is heteroaryl^{a} substituted with NH₂, and optionally substituted with 1 or 2 further substituents as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

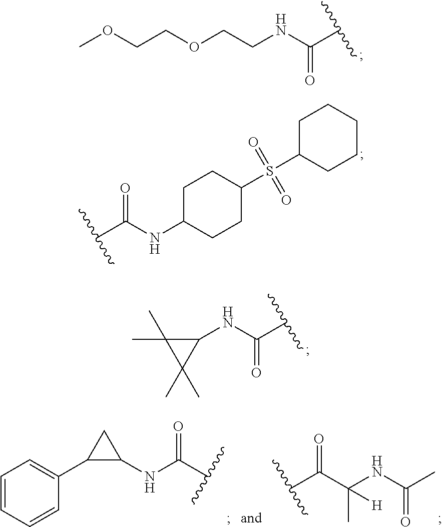

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is isoquinolinyl, optionally substituted as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

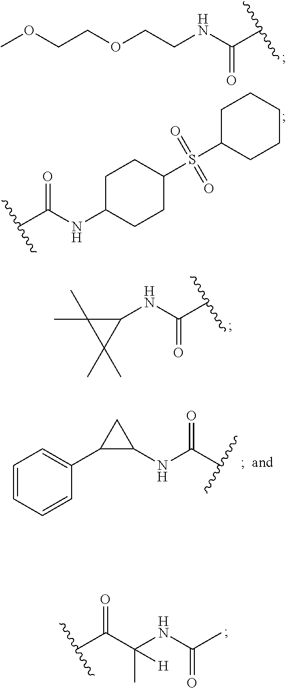

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is

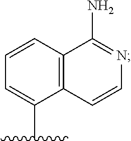 [SMILES: *c1cccc2c(N)nccc12]

the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

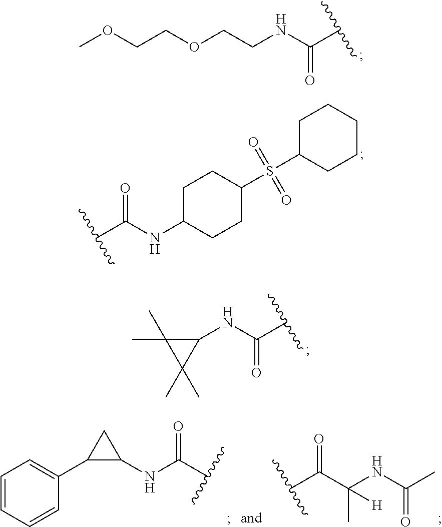

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

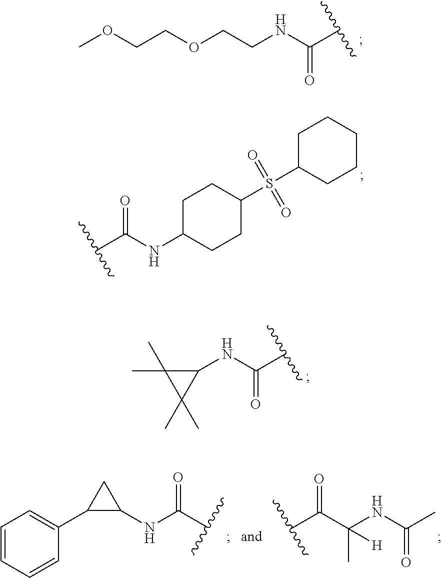

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is heteroaryl^{a} substituted with NH₂, and optionally substituted with 1 or 2 further substituents as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

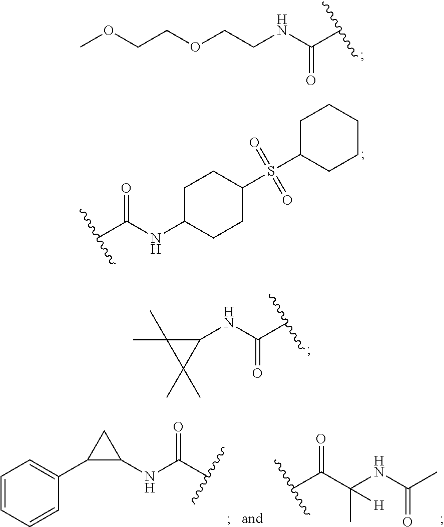

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, B is isoquinolinyl, optionally substituted as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

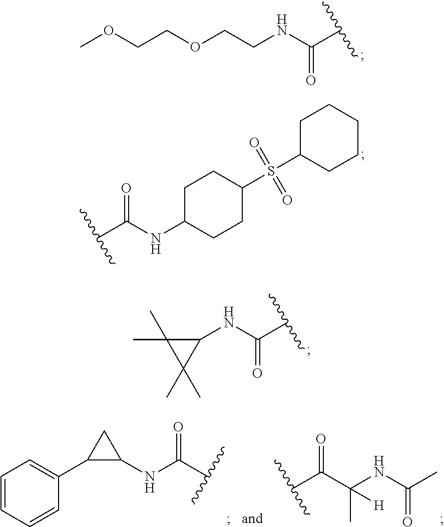

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.
preferably, B is

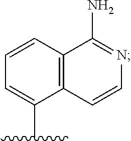 [SMILES: *c1cccc2c(N)nccc12]

Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

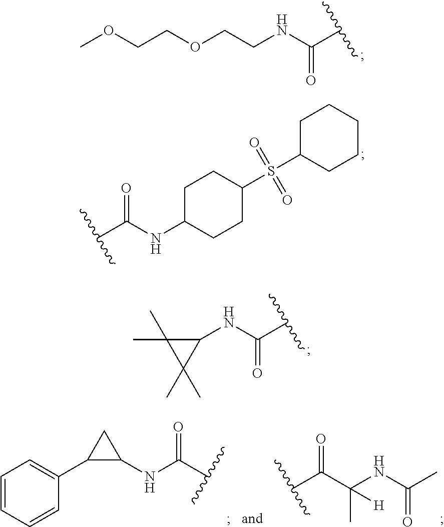

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

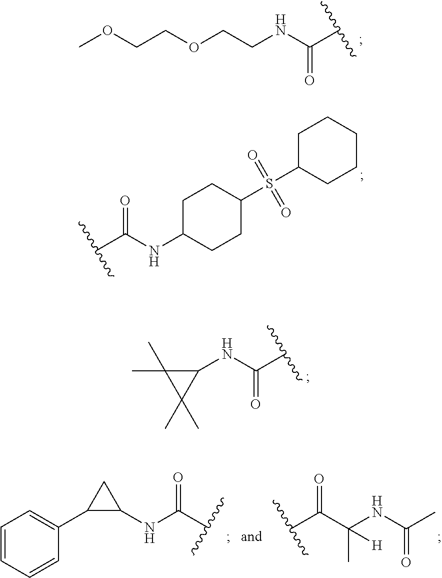

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is heteroaryl^{a} substituted with NH₂, and optionally substituted with 1 or 2 further substituents as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

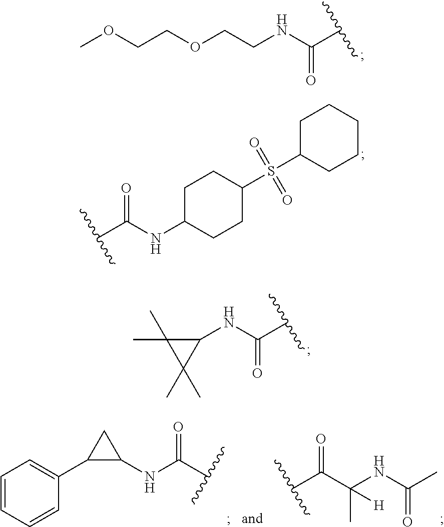

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is isoquinolinyl, optionally substituted as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

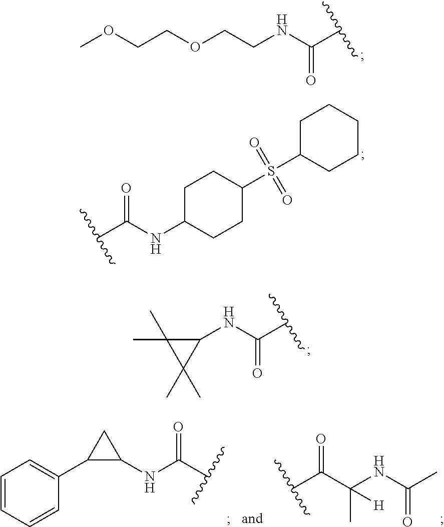

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is

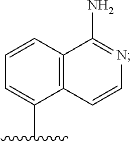 [SMILES: *c1cccc2c(N)nccc12]

the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

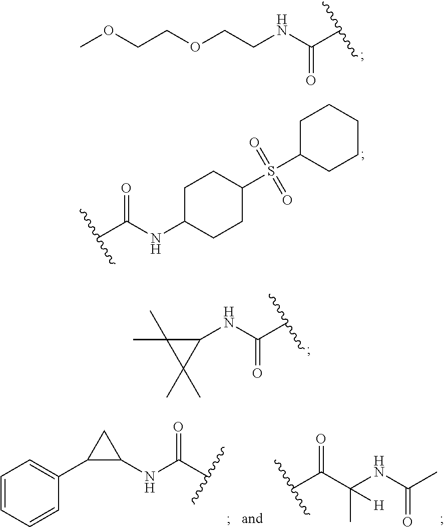

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

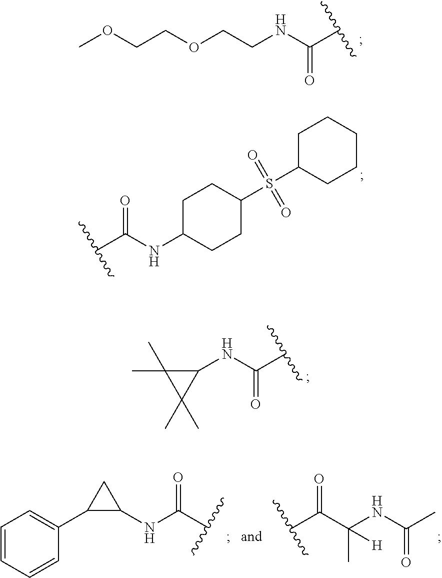

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

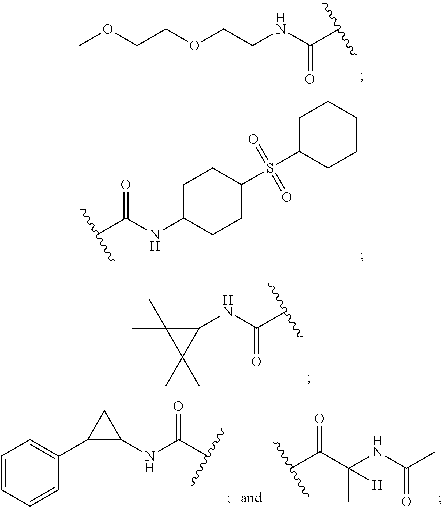

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is heteroaryl^{a} substituted with NH₂, and optionally substituted with 1 or 2 further substituents as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

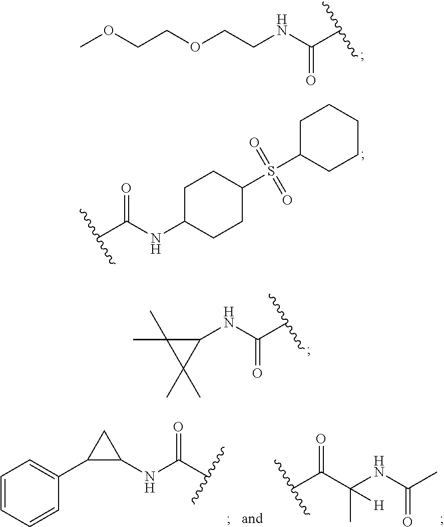

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is isoquinolinyl, optionally substituted as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

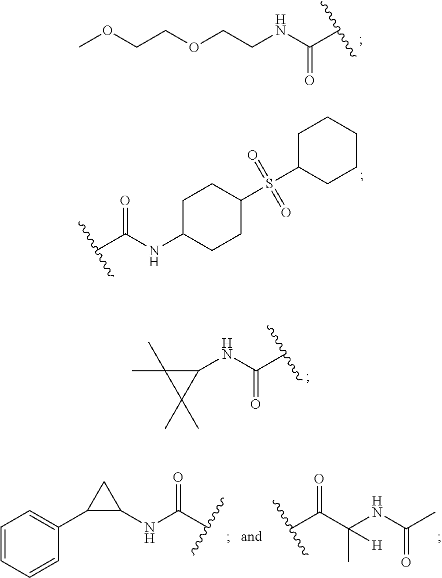

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

Preferably, R16 and R17 are both H; B is

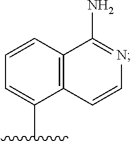 [SMILES: *c1cccc2c(N)nccc12]

Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

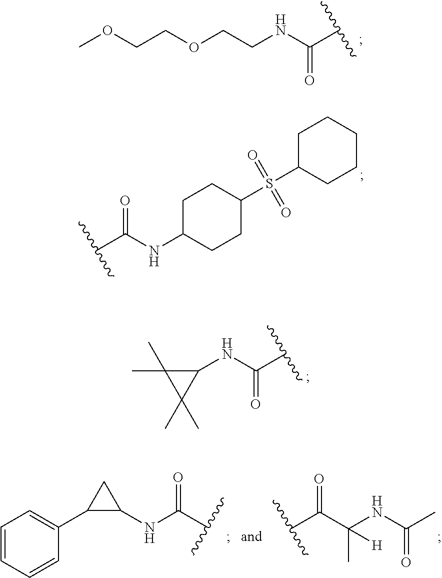

or a compound of formula (Ib) and R23 is C(═O)(CH₂)₁₋₂cycloalkyl; and R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

When V is —N(R23)-, and Z is selected from —C(R16)(R17)-CH₂— and —C(R16)(R17)-; or,
V is selected from —CH₂—C(R16)(R17)- and —C(R16)(R17)-, and Z is —N(R18)-; AW— can be —(CH₂)—O-A.

When V is —N(R23)-, and Z is selected from —C(R16)(R17)-CH₂— and —C(R16)(R17)-; or,
V is selected from —CH₂—C(R16)(R17)- and —C(R16)(R17)-, and Z is —N(R18)-; AW— can be —(CH₂)—O-A.

When V is —N(R23)-, and Z is selected from —C(R16)(R17)-CH₂— and —C(R16)(R17)-; or,
V is selected from —CH₂—C(R16)(R17)- and —C(R16)(R17)-, and Z is —N(R18)-; A can be a 4- to 15-membered mono-, bi-, or tri-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, and CN;
wherein when A is a bicyclic ring system, the bicyclic ring system is fused, bridged or spiro;
wherein when A is a tricyclic ring system, each of the three rings in the tricyclic ring system is either fused, bridged or spiro to at least one of the other rings in the tricyclic ring system.

When V is —N(R23)-, and Z is selected from —C(R16)(R17)-CH₂— and —C(R16)(R17)-; or,
V is selected from —CH₂—C(R16)(R17)- and —C(R16)(R17)-, and Z is —N(R18)-; A can be a 6 to 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein when A is a bicyclic ring system, the bicyclic ring system is fused, bridged or spiro.

When V is —N(R23)-, and Z is selected from —C(R16)(R17)-CH₂— and —C(R16)(R17)-; or,
V is selected from —CH₂—C(R16)(R17)- and —C(R16)(R17)-, and Z is —N(R18)-; A can be a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein when A is a bicyclic ring system, the bicyclic ring system is fused.

When V is —N(R23)-, and Z is selected from —C(R16)(R17)-CH₂— and —C(R16)(R17)-; or,
V is selected from —CH₂—C(R16)(R17)- and —C(R16)(R17)-, and Z is —N(R18)-; A can be substituted, where possible, with 1, 2 or 3 substituents independently selected from alkyl and oxo.

When V is —N(R23)-, and Z is selected from —C(R16)(R17)-CH₂— and —C(R16)(R17)-; or,
V is selected from —CH₂—C(R16)(R17)- and —C(R16)(R17)-, and Z is —N(R18)-; A can be selected from:

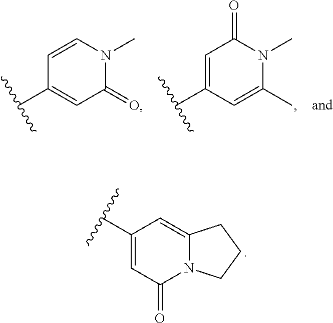

Preferably, R16 and R17 are both H; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, Y is —NH—; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a}; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a} substituted with NH₂, and optionally substituted with 1 or 2 further substituents as for heteroaryl^{a}; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is isoquinolinyl, optionally substituted as for heteroaryl^{a}; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is

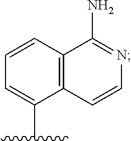 [SMILES: *c1cccc2c(N)nccc12]

A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

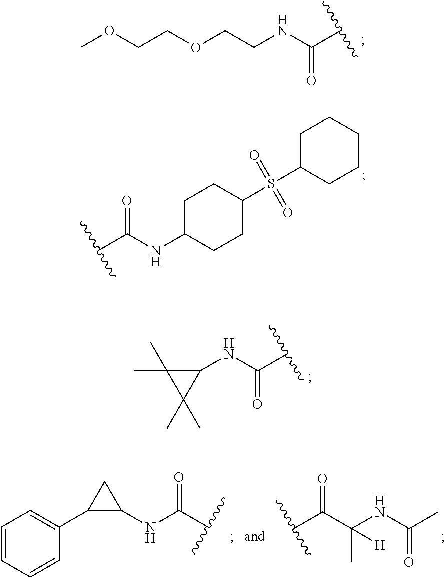

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a}; Y is —NH—; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a} substituted with NH₂, and optionally substituted with 1 or 2 further substituents as for heteroaryl^{a}; Y is —NH—; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is isoquinolinyl, optionally substituted as for heteroaryl^{a}; Y is —NH—; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is

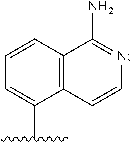 [SMILES: *c1cccc2c(N)nccc12]

Y is —NH—; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a}; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a} substituted with NH₂, and optionally substituted with 1 or 2 further substituents as for heteroaryl^{a}; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is isoquinolinyl, optionally substituted as for heteroaryl^{a}; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is

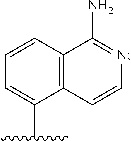 [SMILES: *c1cccc2c(N)nccc12]

A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; Y is —NH—; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a}; Y is —NH—; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a} substituted with NH₂, and optionally substituted with 1 or 2 further substituents as for heteroaryl^{a}; Y is —NH—; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is isoquinolinyl, optionally substituted as for heteroaryl^{a}; Y is —NH—; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is

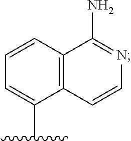 [SMILES: *c1cccc2c(N)nccc12]

Y is —NH—; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

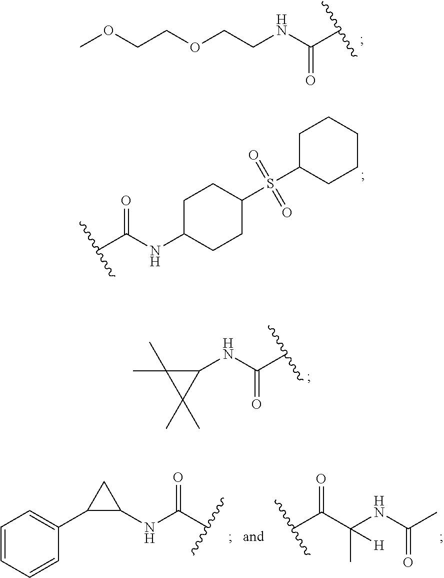

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

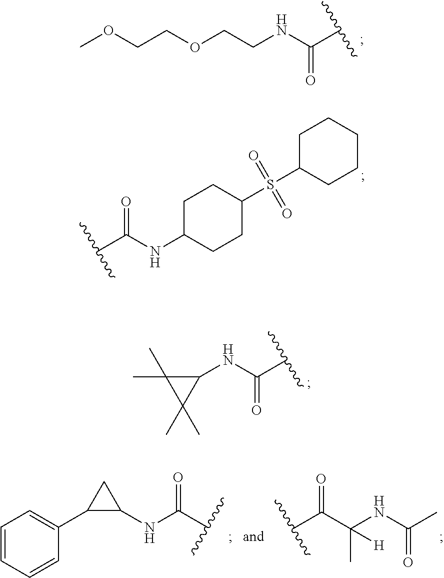

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

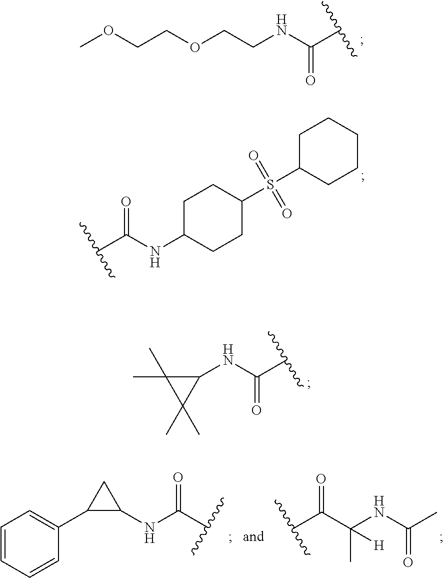

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a} substituted with NH₂, and optionally substituted with 1 or 2 further substituents as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

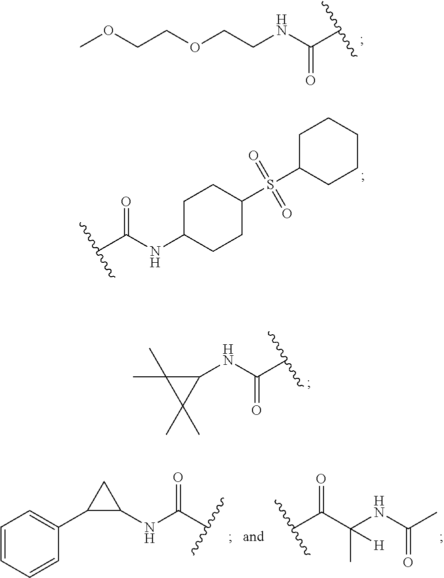

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is isoquinolinyl, optionally substituted as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

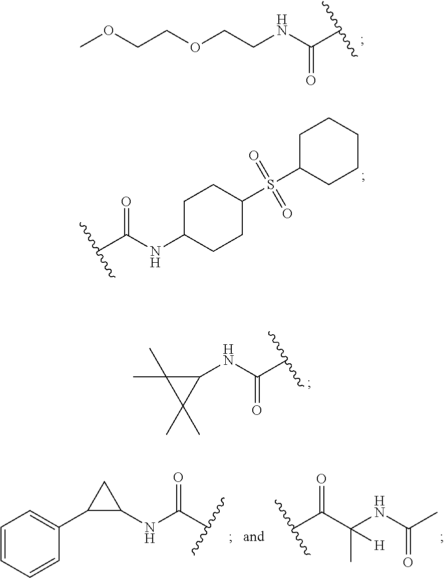

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is

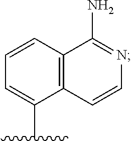 [SMILES: *c1cccc2c(N)nccc12]

the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

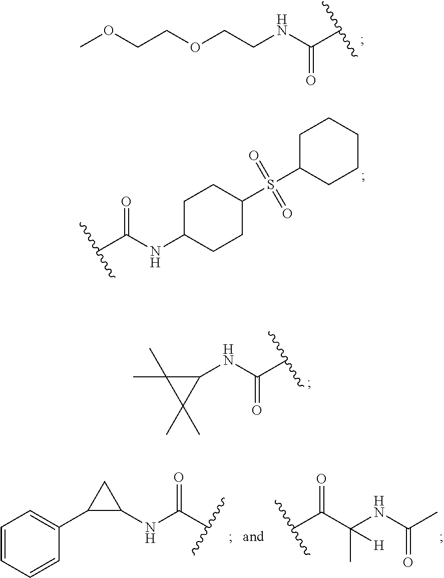

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

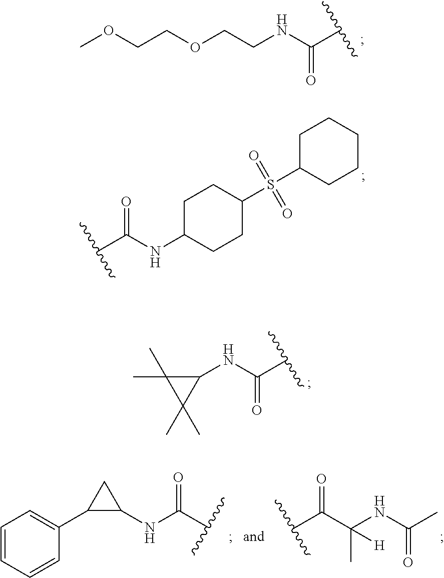

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a} substituted with NH₂, and optionally substituted with 1 or 2 further substituents as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

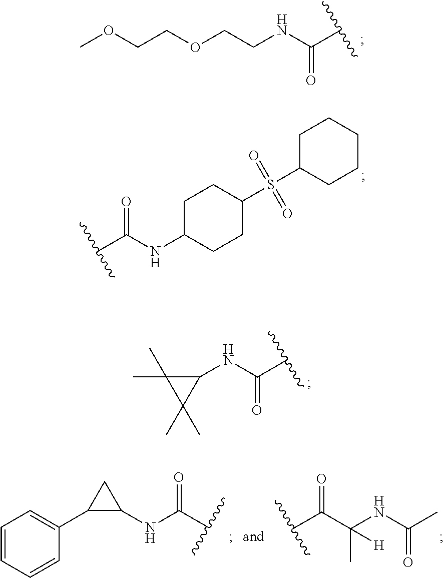

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is isoquinolinyl, optionally substituted as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

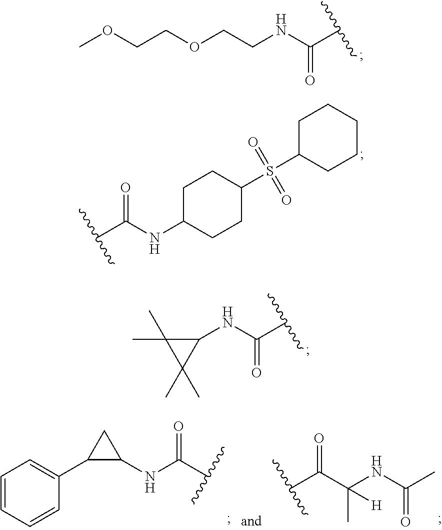

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.
preferably, B is

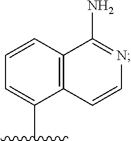 [SMILES: *c1cccc2c(N)nccc12]

Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

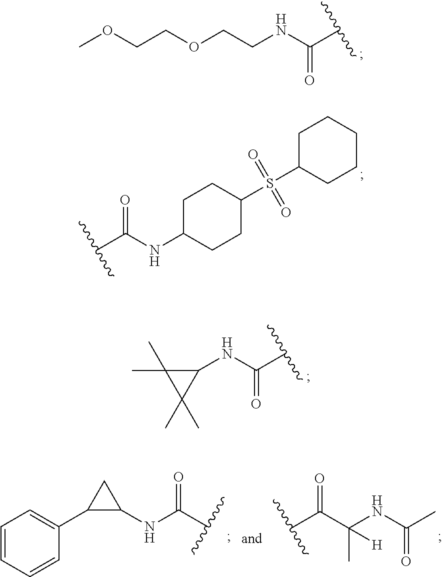

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

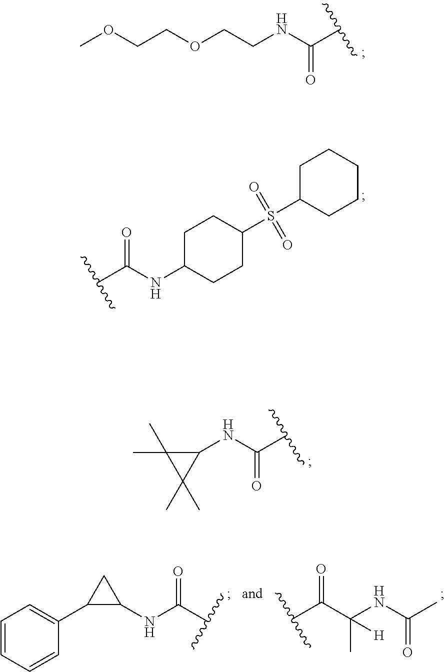

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a} substituted with NH₂, and optionally substituted with 1 or 2 further substituents as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

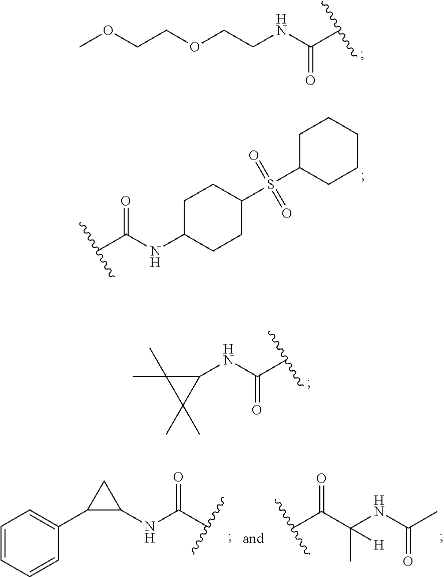

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is isoquinolinyl, optionally substituted as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

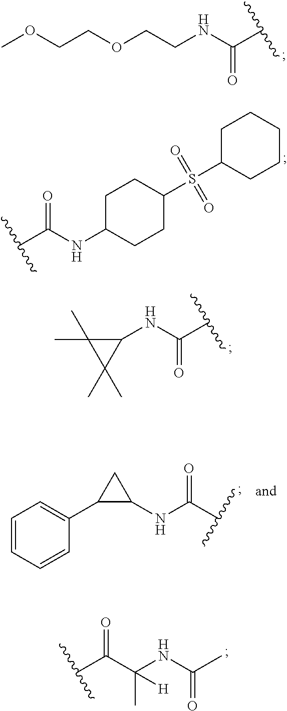

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

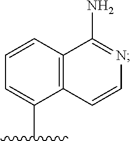 [SMILES: *c1cccc2c(N)nccc12]

Preferably, R16 and R17 are both H; B is; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

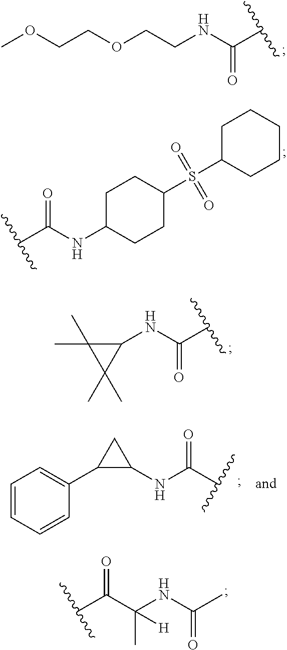

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)-O-A.

Preferably, R16 and R17 are both H; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

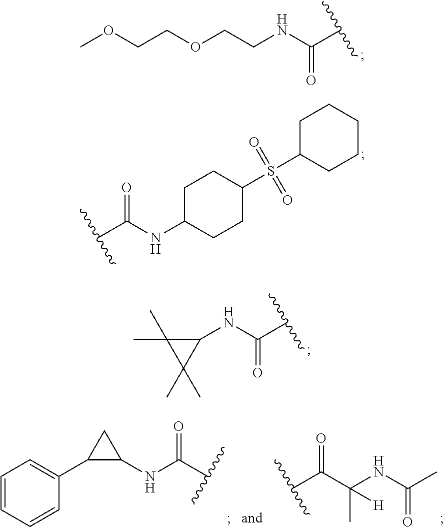

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

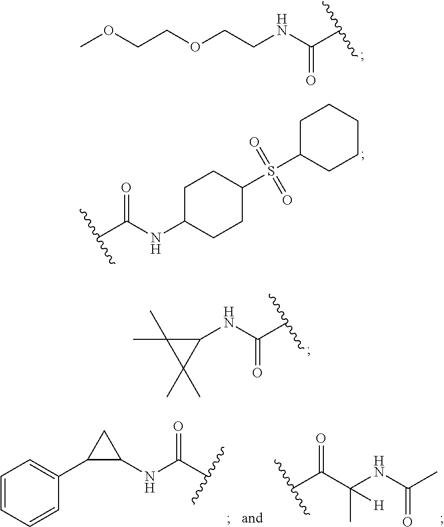

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a} substituted with NH₂, and optionally substituted with 1 or 2 further substituents as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

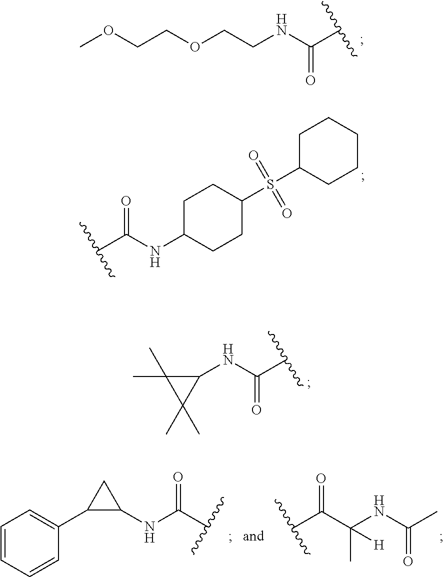

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)-O-A.

Preferably, R16 and R17 are both H; B is isoquinolinyl, optionally substituted as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

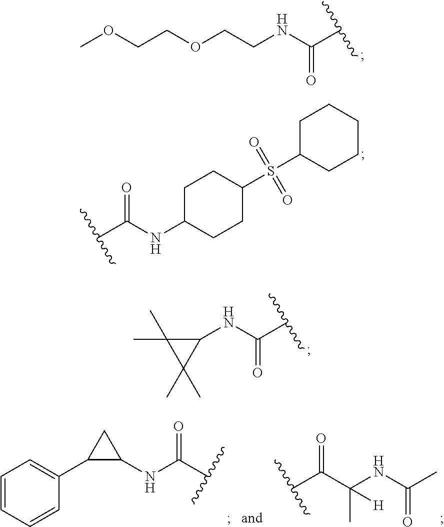

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is

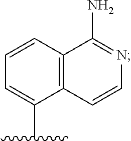 [SMILES: *c1cccc2c(N)nccc12]

Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

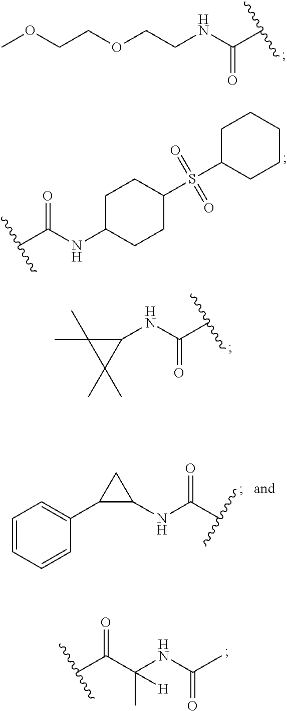

or a compound of formula (Ib) and R23 is C(═O)(CH₂)₁₋₂cycloalkyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, Y is —NH—; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a}; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a} substituted with NH₂, and optionally substituted with 1 or 2 further substituents as for heteroaryl^{a}; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is isoquinolinyl, optionally substituted as for heteroaryl^{a}; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and

Preferably, B is

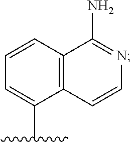 [SMILES: *c1cccc2c(N)nccc12]

R19 is selected from (CH₂)₁₋₂-cycloalkyl, (CH₂)₀₋₁polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

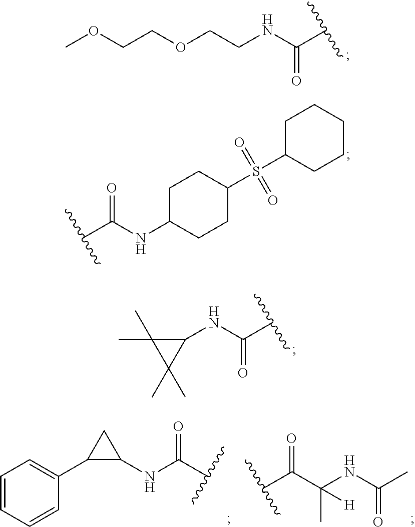

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a}; Y is —NH—; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a} substituted with NH₂, optionally substituted with 1 or 2 further substituents as for heteroaryl^{a}; Y is —NH—; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is isoquinolinyl, optionally substituted as for heteroaryl^{a}; Y is —NH—; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and

Preferably, B is

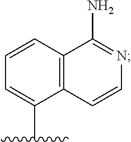 [SMILES: *c1cccc2c(N)nccc12]

Y is —NH—; R19 is selected from (CH₂)₁₋₂-cycloalkyl, (CH₂)₀₋₁polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a}; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a} substituted with NH₂, and optionally substituted with 1 or 2 further substituents as for heteroaryl^{a}; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is isoquinolinyl, optionally substituted as for heteroaryl^{a}; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is

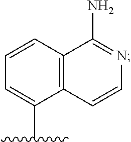 [SMILES: *c1cccc2c(N)nccc12]

R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; Y is —NH—; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a}; Y is —NH—; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a} substituted with NH₂, and optionally substituted with 1 or 2 further substituents as for heteroaryl^{a}; Y is —NH—; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is isoquinolinyl, optionally substituted as for heteroaryl^{a}; Y is —NH—; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is

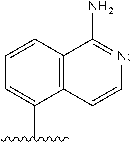 [SMILES: *c1cccc2c(N)nccc12]

Y is —NH—; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

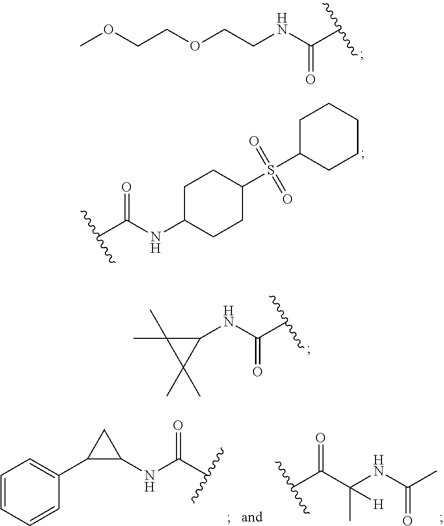

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

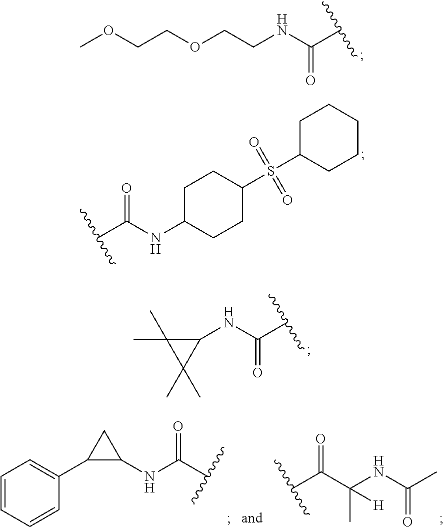

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

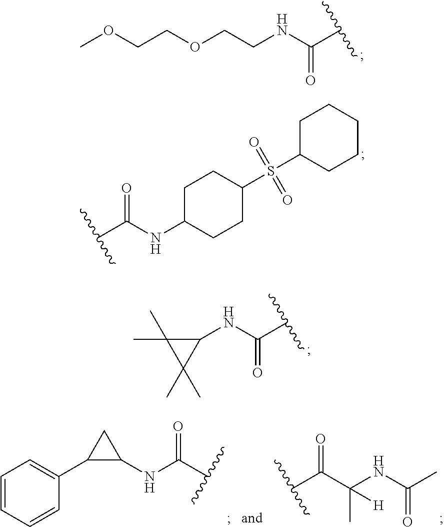

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a} substituted with NH₂, and optionally substituted with 1 or 2 further substituents as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

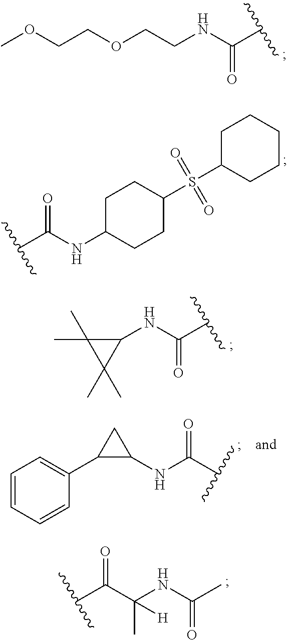

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is isoquinolinyl, optionally substituted as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

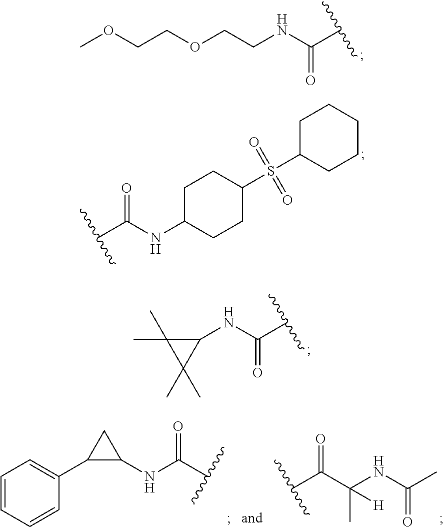

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is

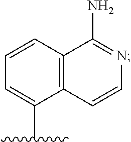 [SMILES: *c1cccc2c(N)nccc12]

the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

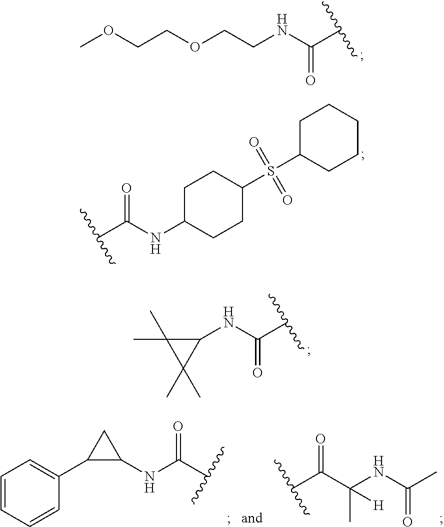

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

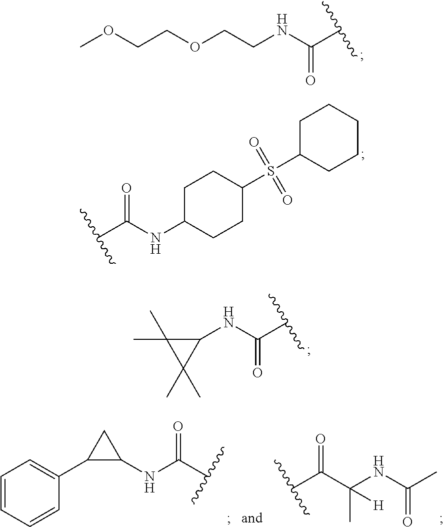

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is heteroaryl^{a} substituted with NH₂, and optionally substituted with 1 or 2 further substituents as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

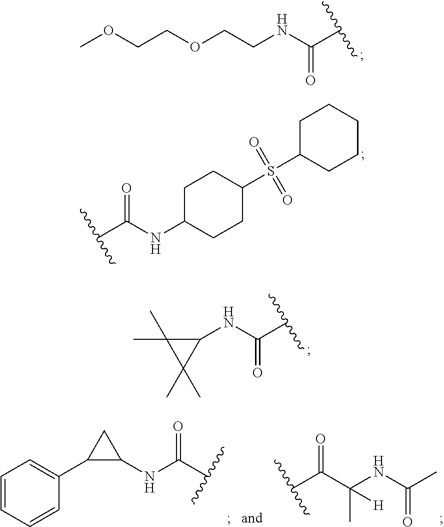

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, B is isoquinolinyl, optionally substituted as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

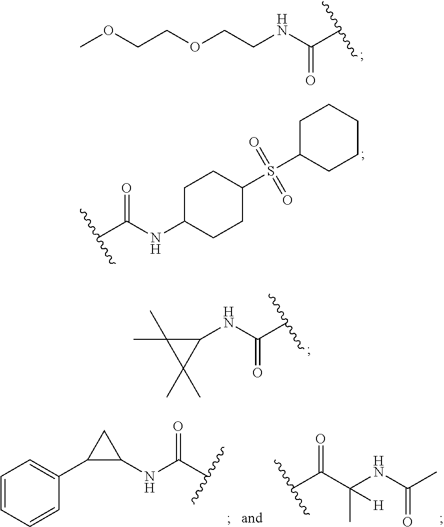

or a compound of formula (Ib) and R23 is C(═O)(CH₂)₁₋₂cycloalkyl: R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.
preferably, B is

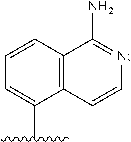 [SMILES: *c1cccc2c(N)nccc12]

Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

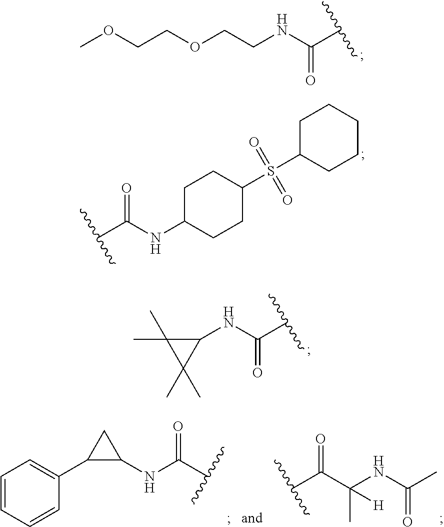

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

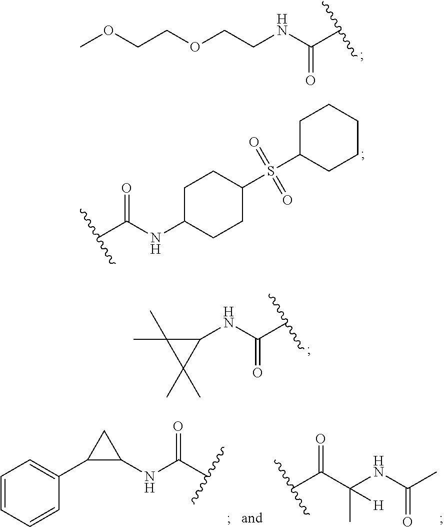

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a} substituted with NH₂, and optionally substituted with 1 or 2 further substituents as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

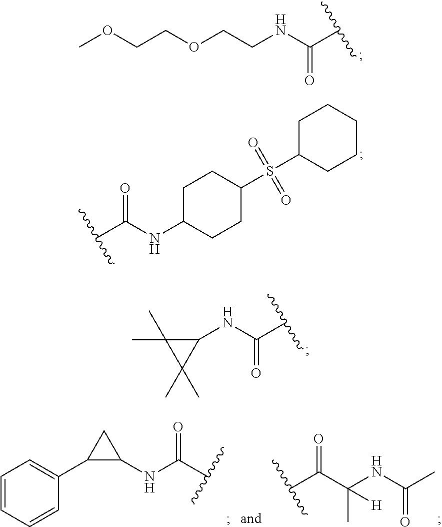

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is isoquinolinyl, optionally substituted as for heteroaryl^{a}; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

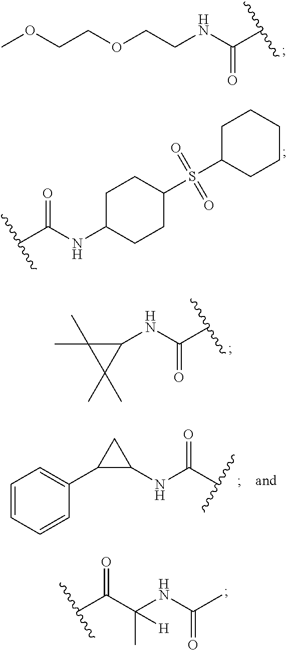

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is

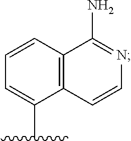 [SMILES: *c1cccc2c(N)nccc12]

the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

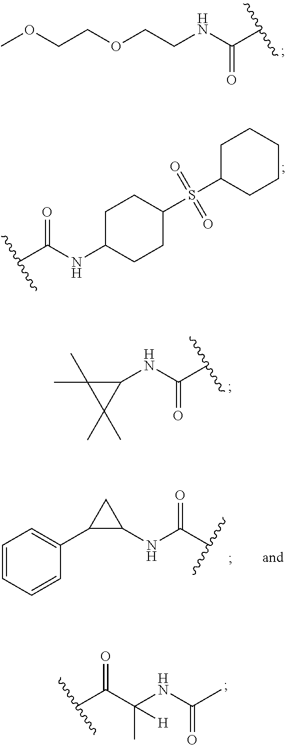

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

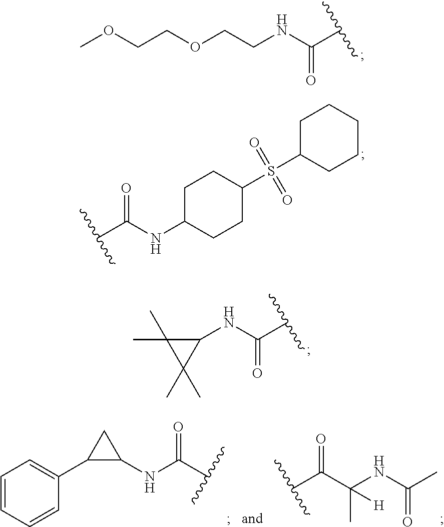

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent:

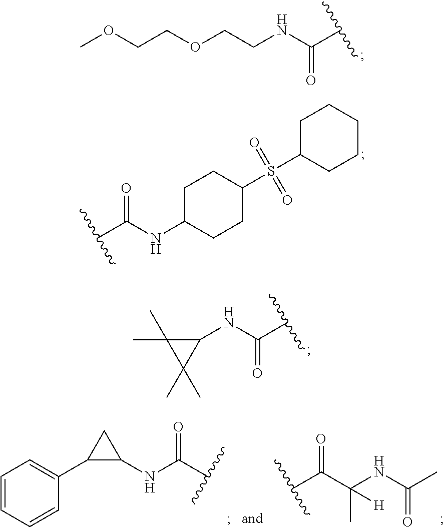

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is heteroaryl^{a} substituted with NH₂, and optionally substituted with 1 or 2 further substituents as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

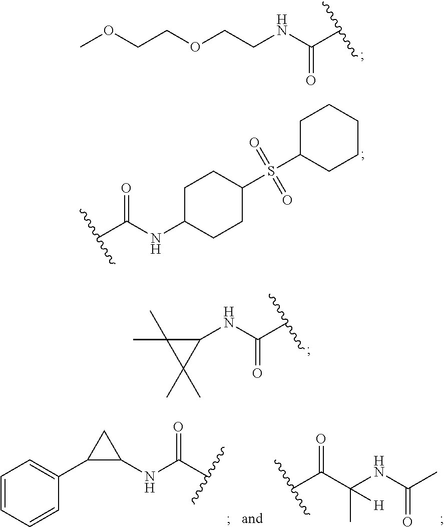

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is isoquinolinyl, optionally substituted as for heteroaryl^{a}; Y is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

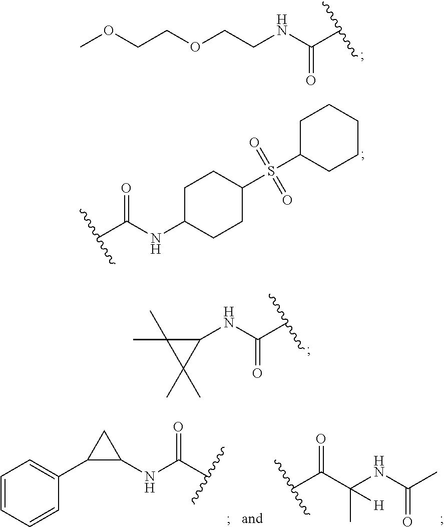

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

Preferably, R16 and R17 are both H; B is

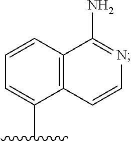 [SMILES: *c1cccc2c(N)nccc12]

is —NH—; the compound of the invention is a compound formula (Ia), wherein R18 can be selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

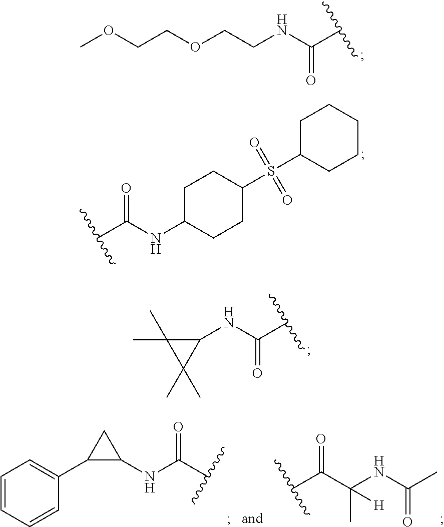

or a compound of formula (Ib) and R23 is C(═O)(CH₂)₁₋₂cycloalkyl; R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl; and A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused; and AW— is —(CH₂)—O-A.

The invention also provides compounds wherein A is A′; wherein
V is —O—, and Z is selected from —C(R16)(R17)-CH₂— and —C(R16)(R17)-; or,
V is selected from —CH₂—C(R16)(R17)- and —C(R16)(R17)-, and Z is —O—.

More specifically, the invention also provides compounds wherein A is A′, wherein the compound is a compound of formula (Ic),

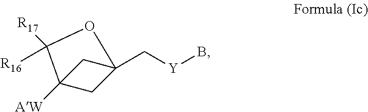

or a tautomer, isomer, stereoisomer (including an enantiomer, a diastereoisomer and a racemic and scalemic mixture thereof), a deuterated analogue, and a pharmaceutically acceptable salt and/or solvate thereof.

When V is —O—, and Z is selected from —C(R16)(R17)-CH₂— and —C(R16)(R17)-; or, V is selected from —CH₂—C(R16)(R17)- and —C(R16)(R17)-, and Z is —O—; wherein A is A′; R16 can be H. Preferably, R16 and R17 can both be H.

When A is A′, A′ can be a 5-membered monocyclic ring system containing one N ring member and optionally one further ring member selected from N and O, wherein the ring system is substituted with phenyl and optionally, where possible, 1, 2, or 3 further substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN, and phenyl.

Preferably, A′ is a 5-membered monocyclic ring system containing one N ring member and optionally one further ring member selected from N and O, wherein the ring system is substituted with phenyl.

More preferably, A′ is selected from;

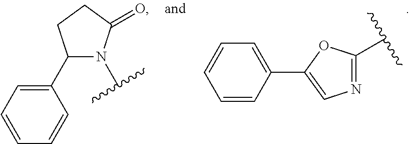 [SMILES: *N1C(=O)CCC1c1ccccc1 ; *c1ncc(-c2ccccc2)o1]

More preferably, A′ is selected from:

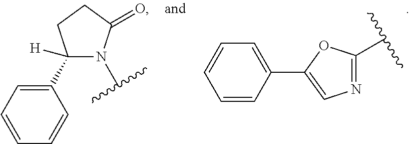 [SMILES: *N1C(=O)CC[C@H]1c1ccccc1 ; *c1ncc(-c2ccccc2)o1]

When AW— is A′W—, A′W— can be —(CH₂)₀₋₃-A′.

More specifically, A′W— can be —(CH₂)₀₋₁-A′.

Preferably, A′W— can be —(CH₂)₀₋₁-A′; and A′ is a 5-membered monocyclic ring system containing one N ring member and optionally one further ring member selected from N and O, wherein the ring system is substituted with phenyl.

Preferably, R16 and R17 are both H; A′W— can be —(CH₂)₀₋₁-A′; and A′ is a 5-membered monocyclic ring system containing one N ring member and optionally one further ring member selected from N and O, wherein the ring system is substituted with phenyl.

Preferably, Y is —NH—; A′W— can be —(CH₂)₀₋₁-A′; and A′ is a 5-membered monocyclic ring system containing one N ring member and optionally one further ring member selected from N and O, wherein the ring system is substituted with phenyl.

Preferably, B is heteroaryl^{a}; A′W— can be —(CH₂)₀₋₁-A′; and A′ is a 5-membered monocyclic ring system containing one N ring member and optionally one further ring member selected from N and O, wherein the ring system is substituted with phenyl.

Preferably, B is heteroaryl^{a} substituted with NH₂, and optionally substituted with 1 or 2 further substituents as for heteroaryl^{a}; A′W— can be —(CH₂)₀₋₁-A′; and A′ is a 5-membered monocyclic ring system containing one N ring member and optionally one further ring member selected from N and O, wherein the ring system is substituted with phenyl.

Preferably, B is isoquinolinyl, optionally substituted as for heteroaryl^{a}; A′W— can be —(CH₂)₀₋₁-A′; and A′ is a 5-membered monocyclic ring system containing one N ring member and optionally one further ring member selected from N and O, wherein the ring system is substituted with phenyl.

Preferably, B is

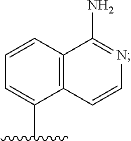 [SMILES: *c1cccc2c(N)nccc12]

A′W— can be —(CH₂)₀₋₁-A′; and A′ is a 5-membered monocyclic ring system containing one N ring member and optionally one further ring member selected from N and O, wherein the ring system is substituted with phenyl.

Preferably, B is heteroaryl^{a}; Y is —NH—; A′W— can be —(CH₂)₀₋₁-A′; and A′ is a 5-membered monocyclic ring system containing one N ring member and optionally one further ring member selected from N and O, wherein the ring system is substituted with phenyl.

Preferably, B is heteroaryl^{a} substituted with NH₂, and optionally substituted with 1 or 2 further substituents as for heteroaryl^{a}; Y is —NH—; A′W— can be —(CH₂)₀₋₁-A′; and A′ is a 5-membered monocyclic ring system containing one N ring member and optionally one further ring member selected from N and O, wherein the ring system is substituted with phenyl.

Preferably, B is isoquinolinyl, optionally substituted as for heteroaryl^{a}; Y is —NH—; A′W— can be —(CH₂)₀₋₁-A′; and A′ is a 5-membered monocyclic ring system containing one N ring member and optionally one further ring member selected from N and O, wherein the ring system is substituted with phenyl.
preferably, B is

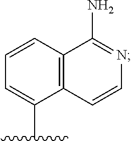 [SMILES: *c1cccc2c(N)nccc12]

Y is —NH—; A′W— can be —(CH₂)₀₋₁-A′; and A′ is a 5-membered monocyclic ring system containing one N ring member and optionally one further ring member selected from N and O, wherein the ring system is substituted with phenyl.

Preferably, R16 and R17 are both H; B is heteroaryl^{a}; A′W— can be —(CH₂)₀₋₁-A′; and A′ is a 5-membered monocyclic ring system containing one N ring member and optionally one further ring member selected from N and O, wherein the ring system is substituted with phenyl.

Preferably, R16 and R17 are both H; B is heteroaryl^{a} substituted with NH₂, and optionally substituted with 1 or 2 further substituents as for heteroaryl^{a}; A′W— can be —(CH₂)₀₋₁-A′; and A′ is a 5-membered monocyclic ring system containing one N ring member and optionally one further ring member selected from N and O, wherein the ring system is substituted with phenyl.

Preferably, R16 and R17 are both H; B is isoquinolinyl, optionally substituted as for heteroaryl^{a}; A′W— can be —(CH₂)₀₋₁-A′; and A′ is a 5-membered monocyclic ring system containing one N ring member and optionally one further ring member selected from N and O, wherein the ring system is substituted with phenyl.

Preferably, R16 and R17 are both H; B is

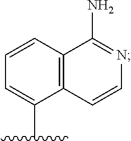 [SMILES: *c1cccc2c(N)nccc12]

A′W— can be —(CH₂)₀₋₁-A′; and A′ is a 5-membered monocyclic ring system containing one N ring member and optionally one further ring member selected from N and O, wherein the ring system is substituted with phenyl.

Preferably, R16 and R17 are both H; Y is —NH—; A′W— can be —(CH₂)₀₋₁-A′; and A′ is a 5-membered monocyclic ring system containing one N ring member and optionally one further ring member selected from N and O, wherein the ring system is substituted with phenyl.

Preferably, R16 and R17 are both H; B is heteroaryl^{a}; Y is —NH—; A′W— can be —(CH₂)₀₋₁-A′; and A′ is a 5-membered monocyclic ring system containing one N ring member and optionally one further ring member selected from N and O, wherein the ring system is substituted with phenyl.

Preferably, R16 and R17 are both H; B is heteroaryl^{a} substituted with NH₂, and optionally substituted with 1 or 2 further substituents as for heteroaryl^{a}; Y is —NH—; A′W— can be —(CH₂)₀₋₁-A′; and A′ is a 5-membered monocyclic ring system containing one N ring member and optionally one further ring member selected from N and O, wherein the ring system is substituted with phenyl.

Preferably, R16 and R17 are both H; B is isoquinolinyl, optionally substituted as for heteroaryl^{a}; Y is —NH—; and A′W— can be —(CH₂)₀₋₁-A′; and A′ is a 5-membered monocyclic ring system containing one N ring member and optionally one further ring member selected from N and O, wherein the ring system is substituted with phenyl.

Preferably, R16 and R17 are both H; B is

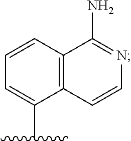 [SMILES: *c1cccc2c(N)nccc12]

Y is —NH—; A′W— can be —(CH₂)₀₋₁-A′; and A′ is a 5-membered monocyclic ring system containing one N ring member and optionally one further ring member selected from N and O, wherein the ring system is substituted with phenyl.

More specifically, the invention provides compounds of formula (I), selected from Table 1, Table 2, Table 3, Table 4, and Table 5, or a pharmaceutically acceptable salt, solvate, or solvate of a salt thereof.

More specifically, the invention provides compounds of formula (I), selected from Table 1; Table 2; compounds 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, and 92 of Table 3; compounds 93, 94, 95, 96, 97, 98, 99, and 101 of Table 4; and Table 5, or a pharmaceutically acceptable salt, solvate, or solvate of a salt thereof.

For the compounds provided in Table 1, Table 2, Table 3, Table 4, and Table 5 below, where stereochemistry is indicated, the compound is intended to cover all possible stereoisomers thereof.

The present invention therefore provides the compounds below in Table 1, and pharmaceutically acceptable salts and/or solvates thereof. The present invention therefore also provides stereoisomers of the compounds below in Table 1, and pharmaceutically acceptable salts and/or solvates thereof.

The present invention therefore provides the compounds below in Table 2, and pharmaceutically acceptable salts and/or solvates thereof. The present invention therefore also provides stereoisomers of the compounds below in Table 2, and pharmaceutically acceptable salts and/or solvates thereof.

The present invention therefore provides the compounds below in Table 3, and pharmaceutically acceptable salts and/or solvates thereof. The present invention therefore also provides stereoisomers of the compounds below in Table 3, and pharmaceutically acceptable salts and/or solvates thereof.

The present invention therefore provides compounds 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, and 92 below in Table 3, and pharmaceutically acceptable salts and/or solvates thereof. The present invention therefore also provides stereoisomers of compounds 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, and 92 below in Table 3, and pharmaceutically acceptable salts and/or solvates thereof.

The present invention therefore provides the compounds below in Table 4, and pharmaceutically acceptable salts and/or solvates thereof. The present invention therefore also provides stereoisomers of the compounds below in Table 4, and pharmaceutically acceptable salts and/or solvates thereof.

The present invention therefore provides compounds 93, 94, 95, 96, 97, 98, 99, and 101 below in Table 4, and pharmaceutically acceptable salts and/or solvates thereof. The present invention therefore also provides stereoisomers of compounds 93, 94, 95, 96, 97, 98, 99, and 101 below in Table 4, and pharmaceutically acceptable salts and/or solvates thereof.

The present invention therefore provides the compounds below in Table 5, and pharmaceutically acceptable salts and/or solvates thereof. The present invention therefore also provides stereoisomers of the compounds below in Table 5, and pharmaceutically acceptable salts and/or solvates thereof.

It will be understood that, when reading the compounds in Table 1, Table 2, Table 3, Table 4, and Table 5 below, the substituents are to be read from left to right. For example, example compound 93 in Table 4 has a Q₁ group:

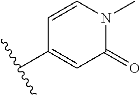 [SMILES: *c1ccn(C)c(=O)c1]

and a Q₂ group “OCH₂”. Therefore, the Q₁ group is attached to the “O” of the “OCH₂” of the Q₂ group, as follows:

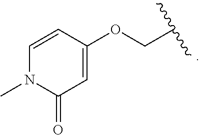 [SMILES: *COc1ccn(C)c(=O)c1]

TABLE 1

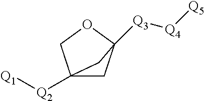

| Example | | | | | |
|---|---|---|---|---|---|
| number | Q₁ | Q₂ | Q₃ | Q₄ | Q₅ |
| 1 | 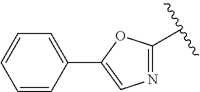 [SMILES: *c1ncc(-c2ccccc2)o1] | Absent | CH₂ | NH | 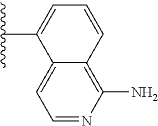 [SMILES: *c1cccc2c(N)nccc12] |
| 2 | 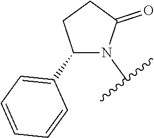 [SMILES: *N1C(=O)CC[C@H]1c1ccccc1] | CH₂ | CH₂ | NH | 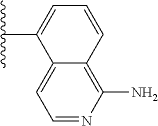 [SMILES: *c1cccc2c(N)nccc12] |

TABLE 2

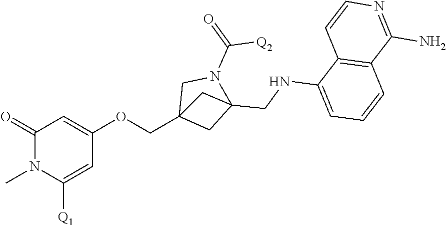

| Example | | |
|---|---|---|
| number | Q₁ | Q₂ |
| 3 | H | 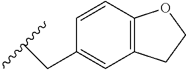 [SMILES: *Cc1ccc2c(c1)CCO2] |
| 4 | H | 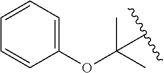 [SMILES: *C(C)(C)Oc1ccccc1] |
| 5 | H | 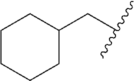 [SMILES: *CC1CCCCC1] |
| 6 | H | 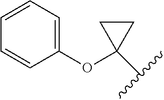 [SMILES: *C1(Oc2ccccc2)CC1] |
| 7 | H | 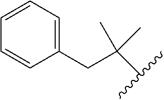 [SMILES: *C(C)(C)Cc1ccccc1] |
| 8 | H | 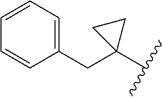 [SMILES: *C1(Cc2ccccc2)CC1] |
| 9 | H | 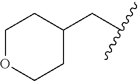 [SMILES: *CC1CCOCC1] |
| 10 | H | 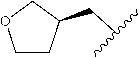 [SMILES: *C[C@H]1CCOC1] |
| 11 | H | 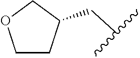 [SMILES: *C[C@@H]1CCOC1] |
| 12 | H | 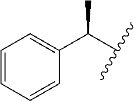 [SMILES: *[C@H](C)c1ccccc1] |
| 13 | H | 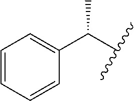 [SMILES: *[C@@H](C)c1ccccc1] |
| 14 | H | 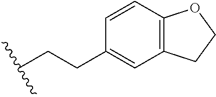 [SMILES: *CCc1ccc2c(c1)CCO2] |
| 15 | H | 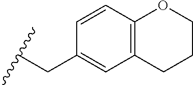 [SMILES: *Cc1ccc2c(c1)CCCO2] |
| 16 | H | 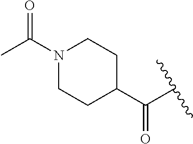 [SMILES: *C(=O)C1CCN(C(C)=O)CC1] |
| 17 | H | 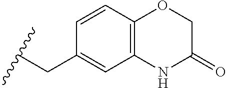 [SMILES: *Cc1ccc2c(c1)NC(=O)CO2] |
| 18 | H | 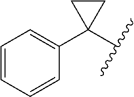 [SMILES: *C1(c2ccccc2)CC1] |
| 19 | H | 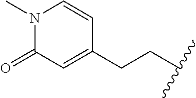 [SMILES: *CCc1ccn(C)c(=O)c1] |
| 20 | CH₃ | 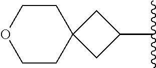 [SMILES: *C1CC2(CCOCC2)C1] |
| 21 | CH₃ | 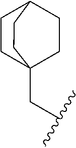 [SMILES: *CC12CCC(CC1)CC2] |
| 22 | CH₃ | 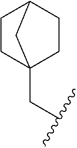 [SMILES: *CC12CCC(CC1)C2] |
| 23 | CH₃ | 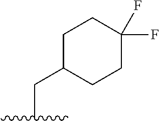 [SMILES: *CC1CCC(F)(F)CC1] |
| 24 | CH₃ | 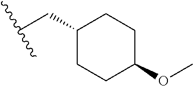 [SMILES: *C[C@H]1CC[C@H](OC)CC1] |
| 25 | CH₃ | 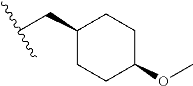 [SMILES: *C[C@H]1CC[C@@H](OC)CC1] |
| 26 | CH₃ | 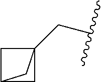 [SMILES: *CC12CC(C1)C2] |
| 27 | H | 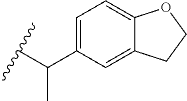 [SMILES: *C(C)c1ccc2c(c1)CCO2] |
| 28 | CH₃ | 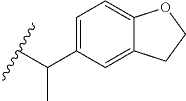 [SMILES: *C(C)c1ccc2c(c1)CCO2] |
| 29 | CH₃ | 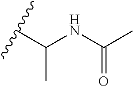 [SMILES: *C(C)NC(C)=O] |

TABLE 3

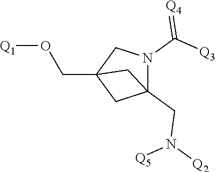

| Example | | | | | |
|---|---|---|---|---|---|
| number | Q₁ | Q₂ | Q₃ | Q₄ | Q₅ |
| 30 | 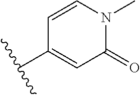 [SMILES: *c1ccn(C)c(=O)c1] | 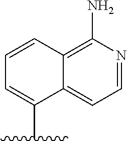 [SMILES: *c1cccc2c(N)nccc12] | 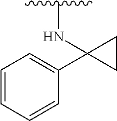 [SMILES: *NC1(c2ccccc2)CC1] | O | H |
| 31 | 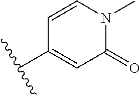 [SMILES: *c1ccn(C)c(=O)c1] | 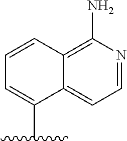 [SMILES: *c1cccc2c(N)nccc12] | 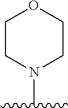 [SMILES: *N1CCOCC1] | O | H |
| 32 | 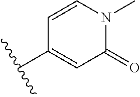 [SMILES: *c1ccn(C)c(=O)c1] | 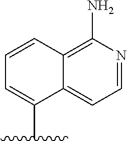 [SMILES: *c1cccc2c(N)nccc12] | 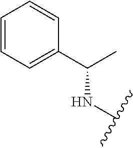 [SMILES: *N[C@@H](C)c1ccccc1] | O | H |
| 33 | 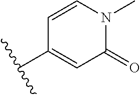 [SMILES: *c1ccn(C)c(=O)c1] | 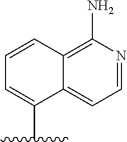 [SMILES: *c1cccc2c(N)nccc12] | 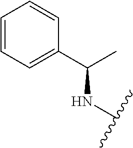 [SMILES: *N[C@H](C)c1ccccc1] | O | H |
| 34 | 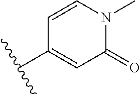 [SMILES: *c1ccn(C)c(=O)c1] | 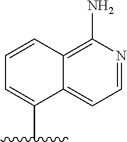 [SMILES: *c1cccc2c(N)nccc12] | 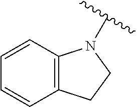 [SMILES: *N1CCc2ccccc21] | O | H |
| 35 | 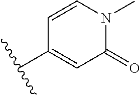 [SMILES: *c1ccn(C)c(=O)c1] | 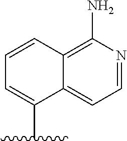 [SMILES: *c1cccc2c(N)nccc12] | 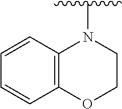 [SMILES: *N1CCOc2ccccc21] | O | H |
| 36 | 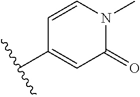 [SMILES: *c1ccn(C)c(=O)c1] | 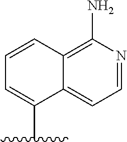 [SMILES: *c1cccc2c(N)nccc12] | 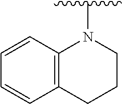 [SMILES: *N1CCCc2ccccc21] | O | H |
| 37 | 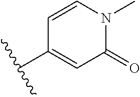 [SMILES: *c1ccn(C)c(=O)c1] | 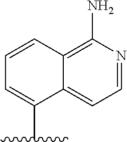 [SMILES: *c1cccc2c(N)nccc12] | 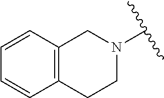 [SMILES: *N1CCc2ccccc2C1] | O | H |
| 38 | 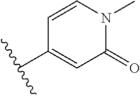 [SMILES: *c1ccn(C)c(=O)c1] | 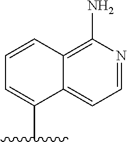 [SMILES: *c1cccc2c(N)nccc12] | 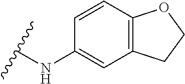 [SMILES: *Nc1ccc2c(c1)CCO2] | O | H |
| 39 | 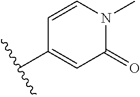 [SMILES: *c1ccn(C)c(=O)c1] | 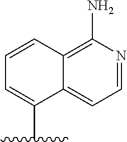 [SMILES: *c1cccc2c(N)nccc12] | 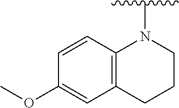 [SMILES: *N1CCCc2cc(OC)ccc21] | O | H |
| 40 | 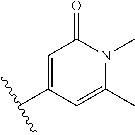 [SMILES: *c1cc(C)n(C)c(=O)c1] | 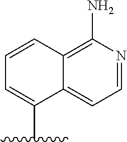 [SMILES: *c1cccc2c(N)nccc12] | 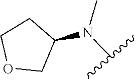 [SMILES: *N(C)[C@@H]1CCOC1] | O | H |
| 41 | 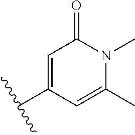 [SMILES: *c1cc(C)n(C)c(=O)c1] | 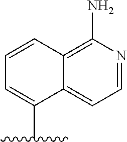 [SMILES: *c1cccc2c(N)nccc12] | 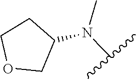 | O | H |
| 42 | 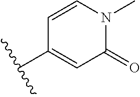 [SMILES: *c1ccn(C)c(=O)c1] | 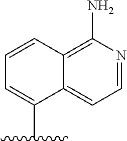 [SMILES: *c1cccc2c(N)nccc12] | 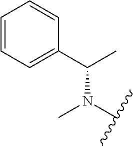 [SMILES: *N(C)[C@@H](C)c1ccccc1] | O | H |
| 43 | 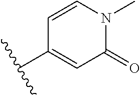 [SMILES: *c1ccn(C)c(=O)c1] | 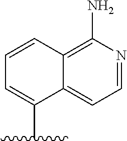 [SMILES: *c1cccc2c(N)nccc12] | 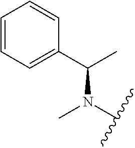 [SMILES: *N(C)[C@H](C)c1ccccc1] | O | H |
| 44 | 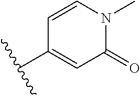 [SMILES: *c1ccn(C)c(=O)c1] | 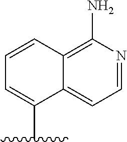 [SMILES: *c1cccc2c(N)nccc12] | 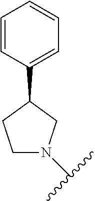 [SMILES: *N1CC[C@@H](c2ccccc2)C1] | O | H |
| 45 | 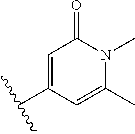 [SMILES: *c1cc(C)n(C)c(=O)c1] | 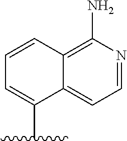 [SMILES: *c1cccc2c(N)nccc12] | 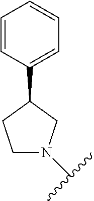 [SMILES: *N1CC[C@@H](c2ccccc2)C1] | O | H |
| 46 | 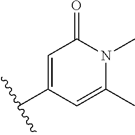 [SMILES: *c1cc(C)n(C)c(=O)c1] | 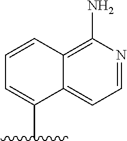 [SMILES: *c1cccc2c(N)nccc12] | 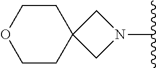 [SMILES: *N1CC2(CCOCC2)C1] | O | H |
| 47 | 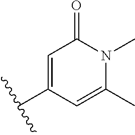 [SMILES: *c1cc(C)n(C)c(=O)c1] | 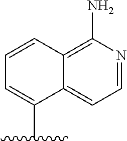 [SMILES: *c1cccc2c(N)nccc12] | 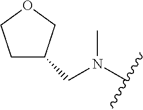 [SMILES: *N(C)C[C@@H]1CCOC1] | O | H |
| 48 | 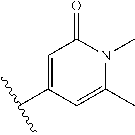 [SMILES: *c1cc(C)n(C)c(=O)c1] | 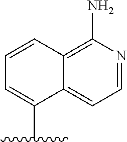 [SMILES: *c1cccc2c(N)nccc12] | 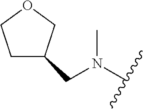 [SMILES: *N(C)C[C@H]1CCOC1] | O | H |
| 49 | 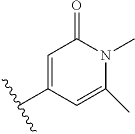 [SMILES: *c1cc(C)n(C)c(=O)c1] | 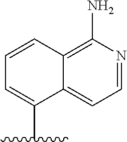 [SMILES: *c1cccc2c(N)nccc12] | 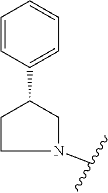 [SMILES: *N1CC[C@H](c2ccccc2)C1] | O | H |
| 50 | 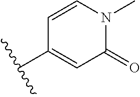 [SMILES: *c1ccn(C)c(=O)c1] | 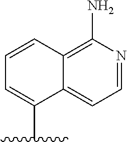 [SMILES: *c1cccc2c(N)nccc12] | 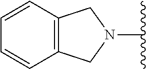 [SMILES: *N1Cc2ccccc2C1] | O | H |
| 51 | 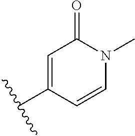 [SMILES: *c1ccn(C)c(=O)c1] | 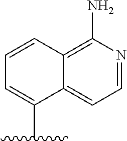 [SMILES: *c1cccc2c(N)nccc12] | 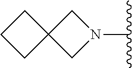 [SMILES: *N1CC2(CCC2)C1] | O | H |
| 52 | 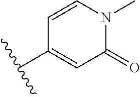 [SMILES: *c1ccn(C)c(=O)c1] | 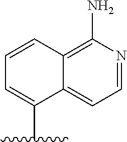 [SMILES: *c1cccc2c(N)nccc12] | 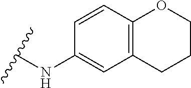 [SMILES: *Nc1ccc2c(c1)CCCO2] | O | H |
| 53 | 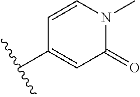 [SMILES: *c1ccn(C)c(=O)c1] | 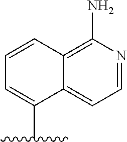 [SMILES: *c1cccc2c(N)nccc12] | 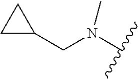 [SMILES: *N(C)CC1CC1] | O | H |
| 54 | 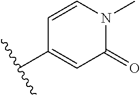 [SMILES: *c1ccn(C)c(=O)c1] | 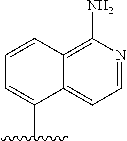 [SMILES: *c1cccc2c(N)nccc12] | 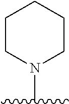 [SMILES: *N1CCCCC1] | O | H |
| 55 | 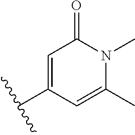 [SMILES: *c1cc(C)n(C)c(=O)c1] | 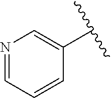 [SMILES: *c1cccnc1] | 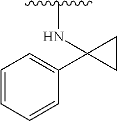 [SMILES: *NC1(c2ccccc2)CC1] | O | H |
| 56 | 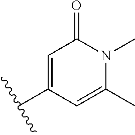 [SMILES: *c1cc(C)n(C)c(=O)c1] | 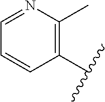 [SMILES: *c1cccnc1C] | 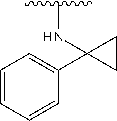 [SMILES: *NC1(c2ccccc2)CC1] | O | H |
| 57 | 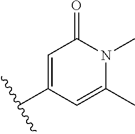 [SMILES: *c1cc(C)n(C)c(=O)c1] | 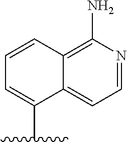 [SMILES: *c1cccc2c(N)nccc12] | 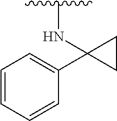 [SMILES: *NC1(c2ccccc2)CC1] | O | H |
| 58 | 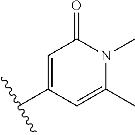 [SMILES: *c1cc(C)n(C)c(=O)c1] | 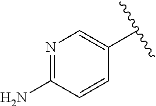 [SMILES: *c1ccc(N)nc1] | 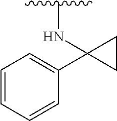 [SMILES: *NC1(c2ccccc2)CC1] | O | H |
| 59 | 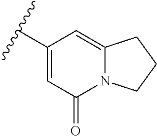 [SMILES: *c1cc2n(c(=O)c1)CCC2] | 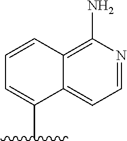 [SMILES: *c1cccc2c(N)nccc12] | 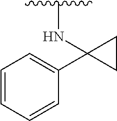 [SMILES: *NC1(c2ccccc2)CC1] | O | H |
| 60 | 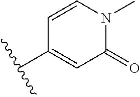 [SMILES: *c1ccn(C)c(=O)c1] | 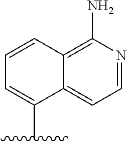 [SMILES: *c1cccc2c(N)nccc12] | 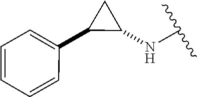 | O | H |
| 61 | 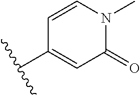 [SMILES: *c1ccn(C)c(=O)c1] | 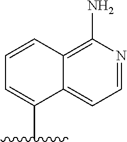 [SMILES: *c1cccc2c(N)nccc12] | 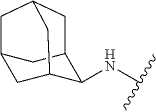 [SMILES: *NC1C2CC3CC(C2)CC1C3] | O | H |
| 62 | 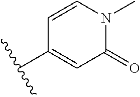 [SMILES: *c1ccn(C)c(=O)c1] | 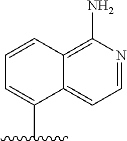 [SMILES: *c1cccc2c(N)nccc12] | 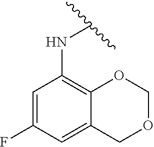 [SMILES: *Nc1cc(F)cc2c1OCOC2] | O | H |
| 63 | 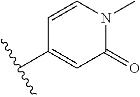 [SMILES: *c1ccn(C)c(=O)c1] | 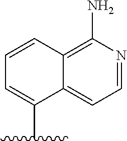 [SMILES: *c1cccc2c(N)nccc12] | 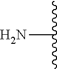 [SMILES: *N] | O | H |
| 64 | 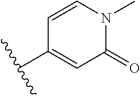 [SMILES: *c1ccn(C)c(=O)c1] | 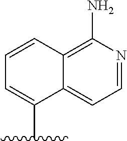 [SMILES: *c1cccc2c(N)nccc12] | 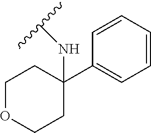 [SMILES: *NC1(c2ccccc2)CCOCC1] | O | H |
| 65 | 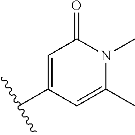 [SMILES: *c1cc(C)n(C)c(=O)c1] | 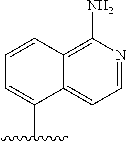 [SMILES: *c1cccc2c(N)nccc12] | 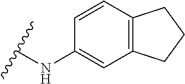 [SMILES: *Nc1ccc2c(c1)CCC2] | O | H |
| 66 | 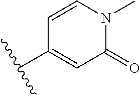 [SMILES: *c1ccn(C)c(=O)c1] | 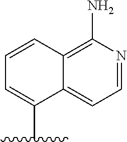 [SMILES: *c1cccc2c(N)nccc12] | 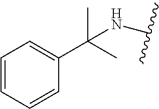 [SMILES: *NC(C)(C)c1ccccc1] | O | H |
| 67 | 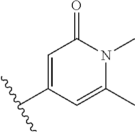 [SMILES: *c1cc(C)n(C)c(=O)c1] | 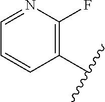 [SMILES: *c1cccnc1F] | 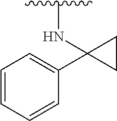 [SMILES: *NC1(c2ccccc2)CC1] | O | H |
| 68 | 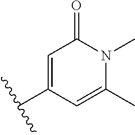 [SMILES: *c1cc(C)n(C)c(=O)c1] | 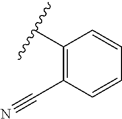 [SMILES: *c1ccccc1C#N] | 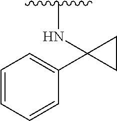 [SMILES: *NC1(c2ccccc2)CC1] | O | H |
| 69 | 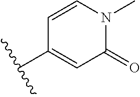 [SMILES: *c1ccn(C)c(=O)c1] | 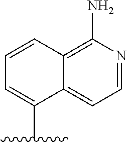 [SMILES: *c1cccc2c(N)nccc12] | 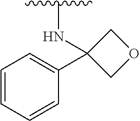 [SMILES: *NC1(c2ccccc2)COC1] | O | H |
| 70 | 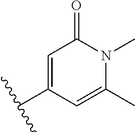 [SMILES: *c1cc(C)n(C)c(=O)c1] | 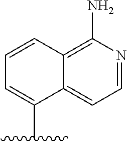 [SMILES: *c1cccc2c(N)nccc12] | 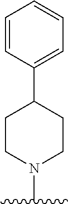 [SMILES: *N1CCC(c2ccccc2)CC1] | O | H |
| 71 | 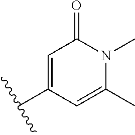 [SMILES: *c1cc(C)n(C)c(=O)c1] | 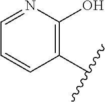 [SMILES: *c1cccnc1O] | 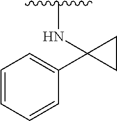 [SMILES: *NC1(c2ccccc2)CC1] | O | H |
| 72 | 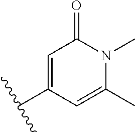 [SMILES: *c1cc(C)n(C)c(=O)c1] | 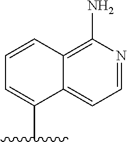 [SMILES: *c1cccc2c(N)nccc12] | 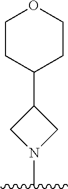 [SMILES: *N1CC(C2CCOCC2)C1] | O | H |
| 73 | 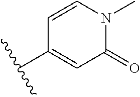 [SMILES: *c1ccn(C)c(=O)c1] | 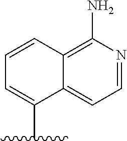 [SMILES: *c1cccc2c(N)nccc12] | 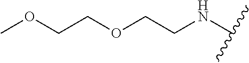 [SMILES: *NCCOCCOC] | O | H |
| 74 | 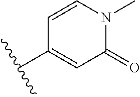 [SMILES: *c1ccn(C)c(=O)c1] | 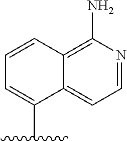 [SMILES: *c1cccc2c(N)nccc12] | 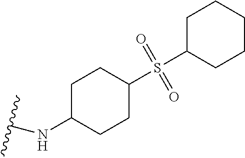 [SMILES: *NC1CCC(S(=O)(=O)C2CCCCC2)CC1] | O | H |
| 75 | 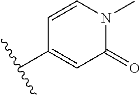 [SMILES: *c1ccn(C)c(=O)c1] | 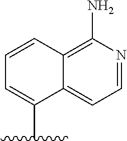 [SMILES: *c1cccc2c(N)nccc12] | 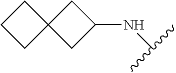 [SMILES: *NC1CC2(CCC2)C1] | O | H |
| 76 | 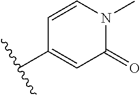 [SMILES: *c1ccn(C)c(=O)c1] | 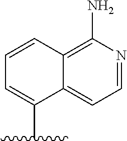 [SMILES: *c1cccc2c(N)nccc12] | 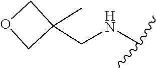 [SMILES: *NCC1(C)COC1] | O | H |
| 77 | 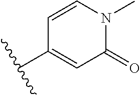 [SMILES: *c1ccn(C)c(=O)c1] | 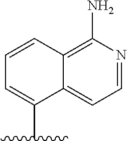 [SMILES: *c1cccc2c(N)nccc12] | 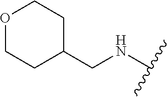 [SMILES: *NCC1CCOCC1] | O | H |
| 78 | 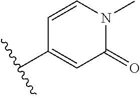 [SMILES: *c1ccn(C)c(=O)c1] | 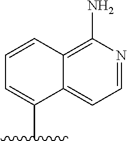 [SMILES: *c1cccc2c(N)nccc12] | 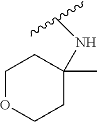 [SMILES: *NC1(C)CCOCC1] | O | H |
| 79 | 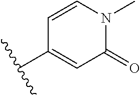 [SMILES: *c1ccn(C)c(=O)c1] | 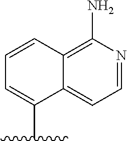 [SMILES: *c1cccc2c(N)nccc12] | 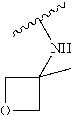 [SMILES: *NC1(C)COC1] | O | H |
| 80 | 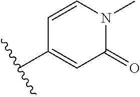 [SMILES: *c1ccn(C)c(=O)c1] | 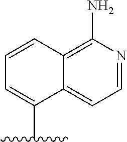 [SMILES: *c1cccc2c(N)nccc12] | 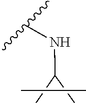 | O | H |
| 81 | 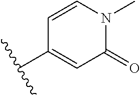 [SMILES: *c1ccn(C)c(=O)c1] | 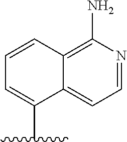 [SMILES: *c1cccc2c(N)nccc12] | 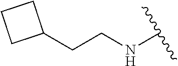 [SMILES: *NCCC1CCC1] | O | H |
| 82 | 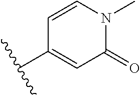 [SMILES: *c1ccn(C)c(=O)c1] | 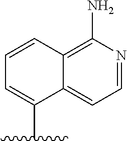 [SMILES: *c1cccc2c(N)nccc12] | 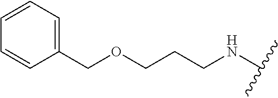 [SMILES: *NCCCOCc1ccccc1] | O | H |
| 83 | 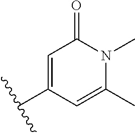 [SMILES: *c1cc(C)n(C)c(=O)c1] | 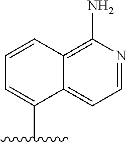 [SMILES: *c1cccc2c(N)nccc12] | 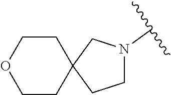 [SMILES: *N1CCC2(CCOCC2)C1] | O | H |
| 84 | 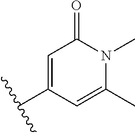 [SMILES: *c1cc(C)n(C)c(=O)c1] | 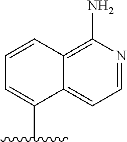 [SMILES: *c1cccc2c(N)nccc12] | 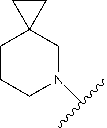 [SMILES: *N1CCCC2(CC2)C1] | O | H |
| 85 | 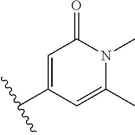 [SMILES: *c1cc(C)n(C)c(=O)c1] | 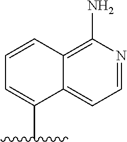 [SMILES: *c1cccc2c(N)nccc12] | 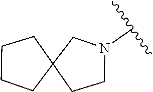 [SMILES: *N1CCC2(CCCC2)C1] | O | H |
| 86 | 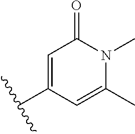 [SMILES: *c1cc(C)n(C)c(=O)c1] | 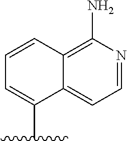 [SMILES: *c1cccc2c(N)nccc12] | 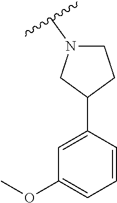 [SMILES: *N1CCC(c2cccc(OC)c2)C1] | O | H |
| 87 | 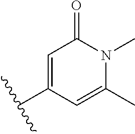 [SMILES: *c1cc(C)n(C)c(=O)c1] | 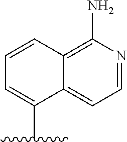 [SMILES: *c1cccc2c(N)nccc12] | 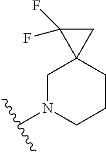 [SMILES: *N1CCCC2(C1)CC2(F)F] | O | H |
| 88 | 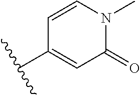 [SMILES: *c1ccn(C)c(=O)c1] | 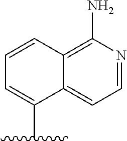 [SMILES: *c1cccc2c(N)nccc12] | 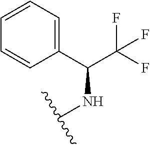 [SMILES: *N[C@@H](c1ccccc1)C(F)(F)F] | O | H |
| 89 | 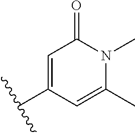 [SMILES: *c1cc(C)n(C)c(=O)c1] | 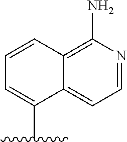 [SMILES: *c1cccc2c(N)nccc12] | 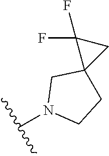 [SMILES: *N1CCC2(C1)CC2(F)F] | O | H |
| 90 | 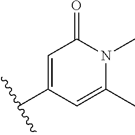 [SMILES: *c1cc(C)n(C)c(=O)c1] | 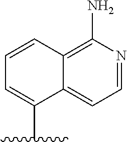 [SMILES: *c1cccc2c(N)nccc12] | 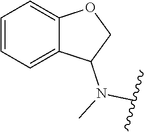 [SMILES: *N(C)C1COc2ccccc21] | O | H |
| 91 | 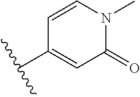 [SMILES: *c1ccn(C)c(=O)c1] | 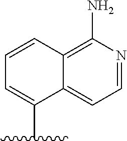 [SMILES: *c1cccc2c(N)nccc12] | 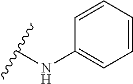 [SMILES: *Nc1ccccc1] | S | H |
| 92 | 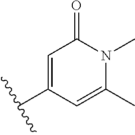 [SMILES: *c1cc(C)n(C)c(=O)c1] | 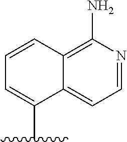 [SMILES: *c1cccc2c(N)nccc12] | 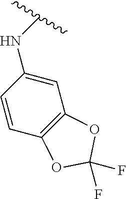 [SMILES: *Nc1ccc2c(c1)OC(F)(F)O2] | O | H |
| 104 | 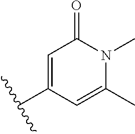 [SMILES: *c1cc(C)n(C)c(=O)c1] | 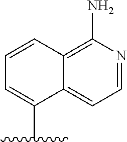 [SMILES: *c1cccc2c(N)nccc12] | 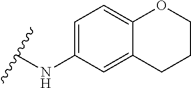 [SMILES: *Nc1ccc2c(c1)CCCO2] | O | CH₃ |

TABLE 4

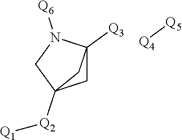

| Example | | | | | | |
|---|---|---|---|---|---|---|
| number | Q₁ | Q₂ | Q₃ | Q₄ | Q₅ | Q₆ |
| 93 | 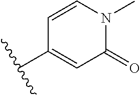 [SMILES: *c1ccn(C)c(=O)c1] | OCH₂ | CH₂ | NH | 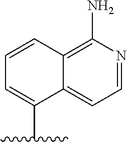 [SMILES: *c1cccc2c(N)nccc12] | 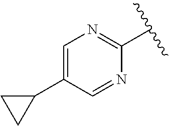 [SMILES: *c1ncc(C2CC2)cn1] |
| 94 | 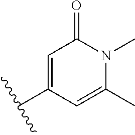 [SMILES: *c1cc(C)n(C)c(=O)c1] | OCH₂ | CH₂ | NH | 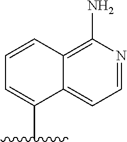 [SMILES: *c1cccc2c(N)nccc12] | 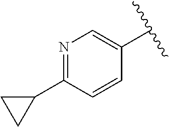 [SMILES: *c1ccc(C2CC2)nc1] |
| 95 | 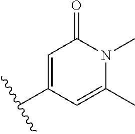 [SMILES: *c1cc(C)n(C)c(=O)c1] | OCH₂ | CH₂ | NH | 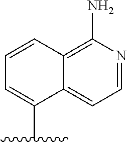 [SMILES: *c1cccc2c(N)nccc12] | 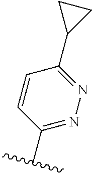 [SMILES: *c1ccc(C2CC2)nn1] |
| 96 | 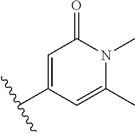 [SMILES: *c1cc(C)n(C)c(=O)c1] | OCH₂ | CH₂ | NH | 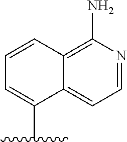 [SMILES: *c1cccc2c(N)nccc12] | 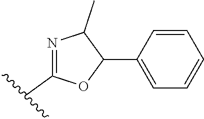 [SMILES: *C1=NC(C)C(c2ccccc2)O1] |
| 97 | 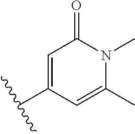 [SMILES: *c1cc(C)n(C)c(=O)c1] | OCH₂ | CH₂ | NH | 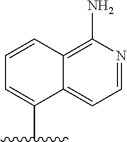 [SMILES: *c1cccc2c(N)nccc12] | 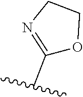 [SMILES: *C1=NCCO1] |
| 98 | 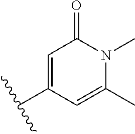 [SMILES: *c1cc(C)n(C)c(=O)c1] | OCH₂ | CH₂ | NH | 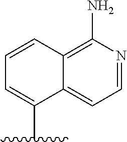 [SMILES: *c1cccc2c(N)nccc12] | 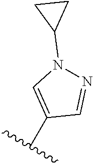 [SMILES: *c1cnn(C2CC2)c1] |
| 99 | 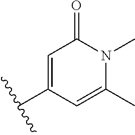 [SMILES: *c1cc(C)n(C)c(=O)c1] | OCH₂ | CH₂ | NH | 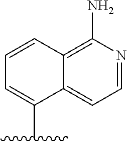 [SMILES: *c1cccc2c(N)nccc12] | 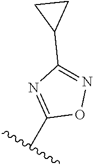 [SMILES: *c1nc(C2CC2)no1] |
| 101 | 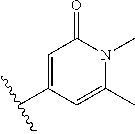 [SMILES: *c1cc(C)n(C)c(=O)c1] | OCH₂ | CH₂ | NH | 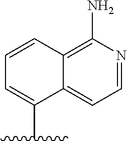 [SMILES: *c1cccc2c(N)nccc12] | 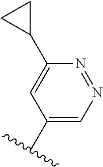 [SMILES: *c1cnnc(C2CC2)c1] |
| 105 | 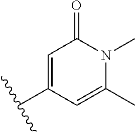 [SMILES: *c1cc(C)n(C)c(=O)c1] | OCH₂ | CH₂ | NH | 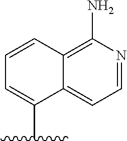 [SMILES: *c1cccc2c(N)nccc12] | 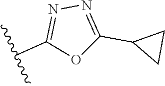 [SMILES: *c1nnc(C2CC2)o1] |

TABLE 5

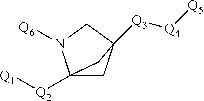

| Example | | | | | | |
|---|---|---|---|---|---|---|
| number | Q₁ | Q₂ | Q₃ | Q₄ | Q₅ | Q₆ |
| 102 | 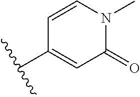 [SMILES: *c1ccn(C)c(=O)c1] | OCH₂ | CH₂ | NH | 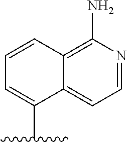 [SMILES: *c1cccc2c(N)nccc12] | 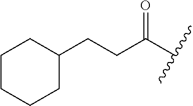 [SMILES: *C(=O)CCC1CCCCC1] |
| 103 | 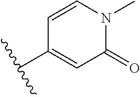 [SMILES: *c1ccn(C)c(=O)c1] | OCH₂ | CH₂ | NH | 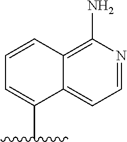 [SMILES: *c1cccc2c(N)nccc12] | 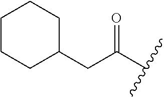 [SMILES: *C(=O)CC1CCCCC1] |

Preferably, the compound of formula (I) is a compound selected from example numbers: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 57, 59, 60, 61, 62, 63, 64, 65, 66, 69, 70, 72, 73, 74, 75, 76, 77, 78, 79, 80, 81, 82, 83, 84, 85, 86, 87, 88, 89, 90, 91, 92, 93, 94, 95, 97, 102, 103, 104, or 105, and pharmaceutically acceptable salts and/or solvates thereof.

Preferably, the compound of formula (I) is a compound selected from example numbers: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 57, 59, 60, 61, 62, 63, 64, 65, 66, 69, 70, 72, 73, 74, 75, 76, 77, 78, 79, 80, 81, 82, 83, 84, 85, 86, 87, 88, 89, 90, 91, 92, 93, 94, 95, 97, 102, or 103, and pharmaceutically acceptable salts and/or solvates thereof.

More preferably, the compound of formula (I) is a compound selected from example numbers: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 57, 59, 60, 61, 62, 63, 64, 65, 66, 69, 70, 72, 73, 74, 75, 76, 77, 78, 79, 80, 81, 82, 83, 84, 85, 86, 87, 88, 89, 90, 91, 92, 93, 94, 95, 97, 102, 104, or 105, and pharmaceutically acceptable salts and/or solvates thereof.

More preferably, the compound of formula (I) is a compound selected from example numbers: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 57, 59, 60, 61, 62, 63, 64, 65, 66, 69, 70, 72, 73, 74, 75, 76, 77, 78, 79, 80, 81, 82, 83, 84, 85, 86, 87, 88, 89, 90, 91, 92, 93, 94, 95, 97, or 102, and pharmaceutically acceptable salts and/or solvates thereof.

Even more preferably, the compound of formula (I) is a compound selected from example numbers: 3, 5, 8, 9, 10, 11, 12, 13, 14, 15, 16, 17, 19, 20, 21, 22, 23, 24, 25, 26, 27, 28, 29, 30, 31, 32, 33, 34, 35, 38, 40, 41, 42, 43, 44, 45, 46, 47, 48, 49, 50, 51, 52, 53, 54, 57, 59, 60, 61, 62, 63, 64, 65, 66, 69, 70, 72, 73, 74, 75, 76, 77, 78, 79, 80, 81, 82, 83, 84, 85, 86, 87, 88, 89, 90, 91, 92, 93, 94, 95, 97, 104, or 105, and pharmaceutically acceptable salts and/or solvates thereof.

Even more preferably, the compound of formula (I) is a compound selected from example numbers: 3, 5, 8, 9, 10, 11, 12, 13, 14, 15, 16, 17, 19, 20, 21, 22, 23, 24, 25, 26, 27, 28, 29, 30, 31, 32, 33, 34, 35, 38, 40, 41, 42, 43, 44, 45, 46, 47, 48, 49, 50, 51, 52, 53, 54, 57, 59, 60, 61, 62, 63, 64, 65, 66, 69, 70, 72, 73, 74, 75, 76, 77, 78, 79, 80, 81, 82, 83, 84, 85, 86, 87, 88, 89, 90, 91, 92, 93, 94, 95, or 97, and pharmaceutically acceptable salts and/or solvates thereof.

Yet more preferably, the compound of formula (I) is a compound selected from example numbers: 3, 15, 17, 30, 38, 43, 44, 45, 52, 57, 59, 60, 62, 65, 66, 69, 90, or 105, and pharmaceutically acceptable salts and/or solvates thereof.

Yet more preferably, the compound of formula (I) is a compound selected from example numbers: 3, 15, 17, 30, 38, 43, 44, 45, 52, 57, 59, 60, 62, 65, 66, 69, or 90, and pharmaceutically acceptable salts and/or solvates thereof. Preferably, the compound of formula (I) is a compound selected from example numbers 104 or 105, and pharmaceutically acceptable salts and/or solvates thereof.

### Therapeutic Applications

As noted above, the compounds (or pharmaceutically acceptable salts and/or solvates thereof), and pharmaceutical compositions comprising the compounds (or pharmaceutically acceptable salts and/or solvates thereof) of the present invention are inhibitors of FXIIa. They are therefore useful in the treatment of disease conditions for which FXIIa is a causative factor.

Accordingly, the present invention provides a compound of the invention (or a pharmaceutically acceptable salt and/or solvate thereof), or a pharmaceutical composition comprising a compound of the invention (or a pharmaceutically acceptable salt and/or solvate thereof), for use in medicine.

The present invention also provides for the use of a compound of the invention (or a pharmaceutically acceptable salt and/or solvate thereof), or a pharmaceutical composition comprising the compound of the invention (or a pharmaceutically acceptable salt and/or solvate thereof), in the manufacture of a medicament for the treatment or prevention of a disease or condition in which FXIIa activity is implicated.

The present invention also provides a method of treatment of a disease or condition in which FXIIa activity is implicated comprising administration to a subject in need thereof a therapeutically effective amount of a compound of the invention (or a pharmaceutically acceptable salt and/or solvate thereof), or a pharmaceutical composition comprising the compound of the invention (or a pharmaceutically acceptable salt and/or solvate thereof).

As discussed above, FXIIa can mediate the conversion of plasma kallikrein from plasma prekallikrein. Plasma kallikrein can then cause the cleavage of high molecular weight kininogen to generate bradykinin, which is a potent inflammatory hormone. Inhibiting FXIIa has the potential to inhibit (or even prevent) plasma kallikrein production. Thus, the disease or condition in which FXIIa activity is implicated can be a bradykinin-mediated angioedema.

The bradykinin-mediated angioedema can be non-hereditary. For example, the non-hereditary bradykinin-mediated angioedema can be selected from non-hereditary angioedema with normal C1 Inhibitor (AE-nC11 nh), which can be environmental, hormonal, or drug-induced; acquired angioedema; anaphylaxis associated angioedema; angiotensin converting enzyme (ACE or ace) inhibitor-induced angioedema; dipeptidyl peptidase-4 inhibitor-induced angioedema; and tPA-induced angioedema (tissue plasminogen activator-induced angioedema).

Alternatively, and preferably, the bradykinin-mediated angioedema can be hereditary angioedema (HAE), which is angioedema caused by an inherited dysfunction/fault/mutation. Types of HAE that can be treated with compounds according to the invention include HAE type 1, HAE type 2, and normal C1 inhibitor HAE (normal C1 nh HAE).

The disease or condition in which FXIIa activity is implicated can be selected from vascular hyperpermeability, stroke including ischemic stroke and haemorrhagic accidents; retinal edema; diabetic retinopathy; impaired visual acuity; DME; retinal vein occlusion; and AMD. These conditions can also be bradykinin-mediated.

As discussed above, FXIIa can activate FXIa to cause a coagulation cascade. Thrombotic disorders are linked to this cascade. Thus, the disease or condition in which FXIIa activity is implicated can be a thrombotic disorder. More specifically, the thrombotic disorder can be thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; acute respiratory distress syndrome (ARDS); idiopathic pulmonary fibrosis (IPF); rheumatoid arthritis (RA); and cold-induced urticarial autoinflammatory syndrome.

Surfaces of medical devices that come into contact with blood can cause thrombosis. The compounds (or pharmaceutically acceptable salts and/or solvates thereof) and pharmaceutical compositions of the present invention can be coated on the surfaces of devices that come into contact with blood to mitigate the risk of the device causing thrombosis. For instance, they can lower the propensity these devices to clot blood and therefore cause thrombosis. Examples of devices that come into contact with blood include vascular grafts, stents, in dwelling catheters, external catheters, orthopedic prosthesis, cardiac prosthesis, and extracorporeal circulation systems.

Other disease conditions for which FXIIa is a causative factor include: neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

Brown adipose tissue (BAT) thermogenic activity can be mediated by the kallikrein-kinin system, and impaired BAT activity is associated with obesity and insulin resistance. Inhibiting FXIIa has the potential to inhibit (or even prevent) BAT activity mediated by the kallikrein-kinin system. The compounds or pharmaceutically acceptable salts and/or solvates thereof) and pharmaceutical compositions of the invention can therefore treat disease conditions such as obesity and diabetes.

Factor XII inhibition is further implicated in the treatment of disease conditions such as kidney disease, renal fibrosis, glomerulosclerosis, renal scarring, ischemia/reperfusion injury in native or transplant kidneys, and acute kidney injury.

### Combination Therapy

The compounds of the present invention (or pharmaceutically acceptable salts and/or solvates thereof) may be administered in combination with other therapeutic agents. Suitable combination therapies include any compound of the present invention (or a pharmaceutically acceptable salt and/or solvate thereof) combined with one or more agents selected from agents that inhibit platelet-derived growth factor (PDGF), endothelial growth factor (VEGF), integrin alpha5beta1, steroids, other agents that inhibit FXIIa and other inhibitors of inflammation.

Some specific examples of therapeutic agents that may be combined with the compounds of the present invention include those disclosed in EP2281885A1 and by S. Patel in Retina, 2009 June; 29(6 Suppl):S45-8.

Other suitable combination therapies include a compound of the invention (or a pharmaceutically acceptable salt and/or solvate thereof) combined with one or more agents selected from agents that treat HAE (as defined generally herein), for example bradykinin B2 antagonists such icatibant (Firazyr®); plasma kallikrein inhibitors such as ecallantide (Kalbitor®), lanadelumab (Takhzyro®) and berotralstat (ORLADEYO™); or C1 esterase inhibitor such as Cinryze® and Haegarda® and Berinert® and Ruconest®.

Other suitable combination therapies include a compound of the invention (or a pharmaceutically acceptable salt and/or solvate thereof) combined with one or more agents selected from agents that are antithrombotics (as outlined above), for example other Factor XIIa inhibitors, thrombin receptor antagonists, thrombin inhibitors, factor VIIa inhibitors, factor Xa inhibitors, factor XIa inhibitors, factor IXa inhibitors, adenosine diphosphate antiplatelet agents (e.g., P2Y12 antagonists), fibrinogen receptor antagonists (e.g. to treat or prevent unstable angina or to prevent reocclusion after angioplasty and restenosis) and aspirin) and platelet aggregation inhibitors.

When combination therapy is employed, the compounds of the present invention and said combination agents may exist in the same or different pharmaceutical compositions, and may be administered separately, sequentially or simultaneously.

The compounds of the present invention can be administered in combination with laser treatment of the retina. The combination of laser therapy with intravitreal injection of an inhibitor of VEGF for the treatment of diabetic macular edema is known (Elman M, Aiello L, Beck R, et al. “Randomized trial evaluating ranibizumab plus prompt or deferred laser or triamcinolone plus prompt laser for diabetic macular edema” Ophthalmology. 27 Apr. 2010).

### DEFINITIONS

As noted above, the term “alkyl” is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from alkoxy, OH, —NR13R14, —C(═O)OR13, —C(═O)NR13R14, CN, CF₃, halo. As noted above “alkyl^{b}”, is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from alkoxy, OH, CN, CF₃, halo. Examples of such alkyl or alkyl^{b} groups include, but are not limited, to C1-methyl, C₂-ethyl, C₃-propyl and C₄-n-butyl, C₃-iso-propyl, C₄-sec-butyl, C₄-iso-butyl, C₄-tert-butyl and C₅-neo-pentyl, optionally substituted as noted above. More specifically, “alkyl” or “alkyl^{b}”, can be a linear saturated hydrocarbon having up to 6 carbon atoms (C₁-C₆) or a branched saturated hydrocarbon of between 3 and 6 carbon atoms (C₃-C₆), optionally substituted as noted above. Even more specifically, “alkyl” or “alkyl^{b}” can be a linear saturated hydrocarbon having up to 4 carbon atoms (C₁-C₄) or a branched saturated hydrocarbon of between 3 and 4 carbon atoms (C₃-C₄), optionally substituted as noted above, which is herein called “small alkyl” or “small alkyl^{b}”, respectively. Preferably, “alkyl” or “alkyl^{b}”, can be defined as a “small alkyl” or “small alkyl^{b}”.

“Aryl” and “aryl^{b}” are as defined above. Typically, “aryl” or “aryl^{b}” will be optionally substituted with 1, 2 or 3 substituents. Optional substituents are selected from those stated above. Examples of suitable aryl or aryl^{b} groups include phenyl, biphenyl and naphthyl (each optionally substituted as stated above). Preferably “aryl” is selected from phenyl, substituted phenyl (wherein said substituents are selected from those stated above) and naphthyl. Most preferably “aryl” is selected from phenyl and substituted phenyl (wherein said substituents are selected from those stated above).

As noted above, the term “cycloalkyl” is a monocyclic saturated hydrocarbon ring of between 3 and 6 carbon atoms (C₃-C₆); cycloalkyl may optionally be substituted with 1 or 2 substituents independently selected from alkyl, alkoxy, OH, CN, CF₃, halo. Examples of suitable monocyclic cycloalkyl groups include cyclopropyl, cyclobutyl, cyclopentyl, and cyclohexyl, optionally substituted as noted above. More specifically, “cycloalkyl” can be a monocyclic saturated hydrocarbon ring of between 3 and 5 carbon atoms, more specifically, between 3 and 4 carbon atoms, optionally substituted as noted above.

As noted above, the term “alkoxy” is —OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted, where possible, with 1 or 2 substituents independently selected from OH, CN, CF₃, and fluoro. Examples of such alkoxy groups include, but are not limited to, C1-methoxy, C₂-ethoxy, C₃-n-propoxy and C₄-n-butoxy for linear alkoxy, and C₃-iso-propoxy, and C₄-sec-butoxy and tert-butoxy for branched alkoxy, optionally substituted as noted above. More specifically, “alkoxy” can be linear groups of between 1 and 4 carbon atoms (C₁-C₄), more specifically, between 1 and 3 carbon atoms (C₁-C₃). More specifically, “alkoxy” can be branched groups of between 3 and 4 carbon atoms (C₃-C₄), optionally substituted as noted above.

“Alkoxy” can be defined as above. Specifically, “alkoxy” is a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from OH, CN, CF₃, and fluoro. Examples of such alkoxy groups include, but are not limited to, C₁-methoxy, C₂-ethoxy, C₃-n-propoxy and C₄-n-butoxy for linear alkoxy, and C₃-iso-propoxy, and C₄-sec-butoxy and tert-butoxy for branched alkoxy, optionally substituted as noted above. More specifically, “alkoxy” can be linear groups of between 1 and 4 carbon atoms (C₁-C₄), more specifically, between 1 and 3 carbon atoms (C₁-C₃). More specifically, “alkoxy” can be branched groups of between 3 and 4 carbon atoms (C₃-C₄), optionally substituted as noted above.

“Halo” can be selected from Cl, F, Br and I. More specifically, halo can be selected from Cl and F.

As noted above, “heteroaryl” is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃. For example, heteroaryl can be selected from thiophene, furan, pyrrole, pyrazole, imidazole, oxazole, isoxazole, thiazole, isothiazole, triazole, oxadiazole, thiadiazole, pyridine, pyridazine, pyrimidine, and pyrazine, optionally substituted as noted above.

“Heteroaryl^{a}” and “heteroaryl^{b}” are as defined above. Typically, “heteroaryl^{a}” or “heteroaryl^{b},” will be optionally substituted with 1, 2 or 3 substituents. Optional substituents are selected from those stated above. Examples of suitable heteroaryl^{a} or heteroaryl^{b} groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl, isoquinolinyl, 5-azathianaphthenyl, indolizinyl, isoindolyl, indazolyl, benzothiazolyl, cinnolinyl, quinazolinyl, quinoxalinyl, 1,8-napthyridinyl and phthalazinyl (optionally substituted as stated above). More specifically, “heteroaryl^{a}” or “heteroaryl^{b}” can be a 9- or 10-membered bi-cyclic ring as defined, and optionally substituted as stated above. Examples of suitable 9- or 10-membered heteroaryl^{a} or heteroaryl^{b} groups include indolyl, benzimidazolyl, benzotriazolyl, quinolinyl, isoquinolinyl, 5-azathianaphthenyl, indolizinyl, isoindolyl, indazolyl, benzothiazolyl, cinnolinyl, quinazolinyl, quinoxalinyl, 1,8-napthyridinyl and phthalazinyl.

As noted above, “heterocycloalkyl” is a non-aromatic carbon-containing monocyclic ring containing 3, 4, 5, or 6, ring members, wherein at least one ring member is independently selected from N, N(R12), S, and O; heterocycloalkyl may be optionally be substituted with 1 or 2 substituents independently selected from alkyl, alkoxy, OH, CN, CF₃, halo. More specifically, “heterocycloalkyl” can be a non-aromatic carbon-containing monocyclic ring containing 3, 4, 5, or 6, ring members, wherein at least one ring member is independently selected from N(R12), and O; heterocycloalkyl may be optionally substituted with 1 or 2 substituents independently selected from alkyl alkoxy, OH, CN, CF₃, halo.

As noted above, “polycycloalkyl” is a bi- or tri-cyclic saturated hydrocarbon ring system of between 5 and 10 carbon atoms (C₅-C₁₀), wherein the bi- or tri-cyclic ring system is bridged or spiro. Examples of such polycycloalkyls include, but are not limited to, adamantane, spiro[3.3]heptane, bicyclo[2.2.2]octane, bicyclo[2.2.1]heptane, and bicyclo[1.1.1]pentane.

As noted above, “heterocycloalkyl^{a}” is a 3-10 membered non-aromatic carbon-containing mono- or bi-cyclic ring system containing one or two ring members independently selected from N and O; heterocycloalkyl^{a} may optionally be substituted with 1 or 2 substituents independently selected from alkyl, alkoxy, aryl, halo, OH, CN, CF₃, and heterocycloalkyl; wherein when heterocycloalkyl^{a} is a bicyclic ring system, the bicyclic ring system is spiro. For example, “heterocycloalkyl^{a}” can be morpholine, tetrahydrofuran, piperidine, 2-azaspiro[3.3]heptane, and 2-azaspiro[4.4]nonane optionally substituted as noted above. More specifically, “heterocycloalkyl^{a}” can be a 4-10 membered non-aromatic carbon-containing mono- or bi-cyclic ring system containing one or two ring members independently selected from N and O. More specifically, heterocycloalkyl^{a} may optionally be substituted with 1 or 2 substituents independently selected from alkyl, aryl, halo, and heterocycloalkyl.

As noted above, “heterocycloalkyl^{b}”, is a 3-6 membered non-aromatic carbon-containing monocyclic ring system which may be saturated or, where possible, unsaturated with 1 or 2 double bonds; heterocycloalkyl^{b} contains one or two ring members independently selected from N, N(C(═O)CH₃) and O; heterocycloalkyl^{b} may optionally be substituted with 1 or 2 substituents independently selected from alkyl, alkoxy, halo, OH, CN, CF₃, phenyl and oxo; wherein the point of attachment of heterocycloalkyl^{b} is not at a ring nitrogen. For example, “heterocycloalkyl^{b}”, can be tetrahydro-2H-pyran, tetrahydrofuran, or oxetane, optionally substituted as noted above. Further, for example, “heterocycloalkyl^{b}”, can be piperidine, wherein the point of attachment is not at the ring nitrogen. For example, if heterocycloalkyl^{b} is piperidine, the attachment of Y to the rest of compound of formula (I) can be at any of the following ring atoms:

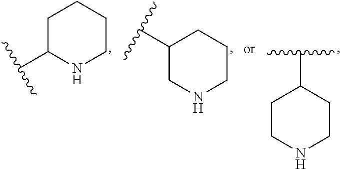

but not at the following ring atom

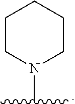 [SMILES: *N1CCCCC1]

“Point of attachment” refers to the point at which heterocycloalkyl^{b} is attached to the rest of the compound of formula (I), it does not refer to the position of any optional substituents. “Heterocycloalkyl^{b}”, can be substituted at a ring nitrogen. For example, heterocycloalkyl^{b} can be

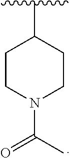 [SMILES: *C1CCN(C(C)=O)CC1]

More specifically, “heterocycloalkyl^{b}” is a 4-6 membered non-aromatic carbon-containing monocyclic ring system. More specifically, “heterocycloalkyl^{b}”, may optionally be substituted with 1 or 2 substituents independently selected from alkyl, phenyl and oxo.

The term “O-linked”, such as in “O-linked hydrocarbon residue”, means that the hydrocarbon residue is joined to the remainder of the molecule via an oxygen atom.

In groups such as —(CH₂)₀₋₆-A, “-” denotes the point of attachment of the substituent group to the remainder of the molecule.

As is clear from the definitions above, and for the avoidance of any doubt, it will be understood that “Y” is defined above, and does not encompass Yttrium.

As is clear from the definitions above, and for the avoidance of any doubt, it will be understood that “B” is defined above, and does not encompass Boron.

As is clear from the definitions above, and for the avoidance of any doubt, it will be understood that “W” is defined above, and does not encompass Tungsten.

As is clear from the definitions above, and for the avoidance of any doubt, it will be understood that “V” is defined above, and does not encompass Vanadium.

“Salt”, as used herein (including “pharmaceutically acceptable salt”) means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, base addition salts (including pharmaceutically acceptable base addition salts) that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, acid addition salts (including pharmaceutically acceptable acid addition salts) that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates, trifluoroacetates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming prodrugs are described in ‘The Practice of Medicinal Chemistry, 2^{nd} Ed. pp561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Where compounds of the invention exist in one or more geometric, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis). For example, where compounds of the invention exist as a mixture of stereoisomers, one stereoisomer can be present at a purity of >90% relative to the remaining stereoisomers, or more specifically at a purity of >95% relative to the remaining stereoisomers, or yet more specifically at a purity of >99% relative to the remaining stereoisomers. For example, where compounds of the invention exists in enantiomeric forms, the compound can be >90% enantiomeric excess (ee), or more specifically >95% enantiomeric excess (ee), or yet more specifically, >99% ee.

Unless otherwise stated, the compounds of the invention include compounds that differ only in the presence of one or more isotopically enriched atoms. For example, compounds wherein hydrogen is replaced by deuterium or tritium, or wherein carbon is replaced by ¹³C or ¹⁴C, are within the scope of the present invention. Such compounds are useful, for example, as analytical tools or probes in biological assays.

Methods for the preparation of deuterated analogues will be readily apparent to those skilled in the art. For example, methods may include the use of deuterated starting materials in the synthesis of the compounds described herein. Deuterated starting materials will be readily available to the skilled person, from standard commercial sources. Methods for making deuterated analogues and deuterated starting materials may also include deuterium exchange. For example, deuterium exchange may be achieved by mixing the compounds with D₂O.

In the context of the present invention, references herein to “treatment” include references to curative, palliative and prophylactic treatment. For instance, treatment includes preventing the symptoms of the disease conditions for which FXIIa is a causative factor.

### Methods

The compounds of the invention may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Compounds of the invention intended for pharmaceutical use may be administered as a solid or liquid, such as a tablet, capsule or solution. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly, the present invention provides a pharmaceutical composition comprising a compound of the invention and a pharmaceutically acceptable carrier, diluent or excipient.

For the treatment of conditions such as retinal vascular permeability associated with diabetic retinopathy and diabetic macular edema, the compounds of the invention may be administered in a form suitable for injection into the ocular region of a patient, in particular, in a form suitable for intra-vitreal injection. It is envisaged that formulations suitable for such use will take the form of sterile solutions of a compound of the invention in a suitable aqueous vehicle. The compositions may be administered to the patient under the supervision of the attending physician.

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, excipients such as sugars (including but not restricted to glucose, manitol, sorbitol, etc.), salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of co-solvents and/or solubility-enhancing agents such as surfactants, micelle structures and cyclodextrins.

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solids and liquids (including multiple phases or dispersed systems). Exemplary formulations suitable for oral administration include tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Liquid (including multiple phases and dispersed systems) formulations include emulsions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, New York, 1980).

For administration to human patients, the total daily dose of the compounds of the invention is typically in the range 0.1 mg and 10,000 mg, or between 1 mg and 5000 mg, or between 10 mg and 1000 mg depending, of course, on the mode of administration.

The total dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

### Numbered Embodiments

1. A compound of Formula (I),

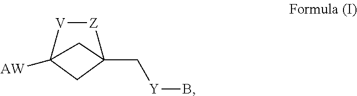

wherein:
V is selected from —O— and —N(R23)-, and Z is selected from —C(R16)(R17)-CH₂— and —C(R16)(R17)-; or,
V is selected from —CH₂—C(R16)(R17)- and —C(R16)(R17)-, and Z is selected from —O— and —N(R18)-; wherein R18 or R23 are selected from C(═S)NH-aryl; C(═S)NH—R19; C(═S)N(alkyl)R19; C(═S)NR13R14; C(═O)R19; C(═O)NHR19; C(═O)N(alkyl)(R19); C(═O)NH₂;
   heterocycloalkyl^{b}; a 5- or 6-membered carbon-containing aromatic ring containing one, two or three ring members independently selected from N and, where possible, O, wherein the carbon-containing aromatic ring is substituted with one substituent selected from cycloalkyl and heterocycloalkyl^{b}, and optionally one or two further substituents independently selected from alkyl, alkoxy, OH, OCF₃, halo, CN, and CF₃;

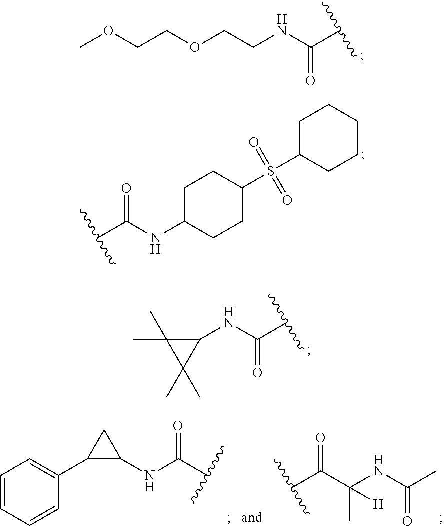

R19 is selected from (CH₂)₁₋₃-cycloalkyl, (CH₂)₀₋₃-polycycloalkyl, heterocycloalkyl^{a}, (CH₂)₁₋₃-heterocycloalkyl^{b}, (CH₂)₀₋₃—R22, (C(R20)(R21))-(CH₂)₀₋₃-phenyl, (C(R20)(R21))-R22, (C(R20)(R21))-O-phenyl, and (CH₂)₁₋₃—O—(CH₂)-phenyl;
R20 and R21 are independently selected from H, alkyl and CF₃, or R20 and R21 together with the carbon atom to which they are attached form a carbon-containing 3-, 4-, 5- or 6-membered saturated ring, optionally containing an O ring member, wherein R20 and R21 are not both H;
R22 is a fused 6,5- or 6,6-bicyclic ring containing an aromatic ring fused to a non-aromatic ring, wherein the bicyclic ring optionally contains one or two ring members selected from N and O, wherein the fused 6,5- or 6,6-bicyclic ring may be optionally substituted with one or two substituents independently selected from alkyl, alkoxy, OH, CN, CF₃, halo, and oxo, wherein the 6,5-bicyclic ring may be attached via the 6- or 5-membered ring, wherein the 6,5-bicyclic ring is not aryl substituted with methylenedioxy, and wherein the 6,6bicyclic ring is not aryl substituted with ethylenedioxy;
Y is selected from —O— and —N(R12)-;
B is selected from:
   (i) heteroaryl^{a}; and
   (ii) aryl;
AW— is selected from:
—(CH₂)₀₋₆—(CHR15)-(CH₂)₀₋₆-A, —(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—N(R12)-(CH₂)₁₋₆—C(═O)-A, —(CH₂)₀₋₆—NH—C(═O)—(CH₂)₀₋₆-A, —C(═O)N(R12)-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A and —SO₂—NH-A;
A is a 4- to 15-membered mono-, bi-, or tri-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN, and phenyl;
wherein when A is a bicyclic ring system, the bicyclic ring system is fused, bridged or spiro;
wherein when A is a tricyclic ring system, each of the three rings in the tricyclic ring system is
either fused, bridged or spiro to at least one of the other rings in the tricyclic ring system;
wherein when:
(i) V is —O—, or
(ii) Z is —O—,
A is A′, wherein A′ is a 4- to 15-membered mono-, bi-, or tri-cyclic ring system, containing one N ring member and optionally one, two or three further ring members independently selected from N, O and S, wherein the ring system is substituted with phenyl and optionally, where possible, 1, 2, or 3 further substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN, and phenyl;
wherein when A′ is a bicyclic ring system, the bicyclic ring system is fused, bridged or spiro; wherein when A′ is a tricyclic ring system, each of the three rings in the tricyclic ring system is either fused, bridged or spiro to at least one of the other rings in the tricyclic ring system;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted, where possible, with 1 or 2 substituents independently selected from OH, CN, CF₃, and fluoro;
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from alkoxy, OH, —NR13R14, —C(═O)OR13, —C(═O)NR13R14, CN, CF₃, halo;
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from alkoxy, OH, CN, CF₃, halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from alkoxy, OH, NR13R14, C(═O)OR13, C(═O)NR13R14, CN, CF₃, halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—O-heteroaryl^{a}, aryl^{b}, —O-aryl^{b}, —(CH₂)₁₋₃-aryl^{b}, —(CH₂)₀₋₃-heteroaryl^{a}, —C(═O)OR13, —C(═O)NR13R14, —(CH₂)₀₋₃—NR13R14, OCF₃ and CF₃;
aryl^{b} is phenyl, biphenyl or naphthyl; aryl^{b} may be optionally substituted with 1, 2 or 3 substituents independently selected from alkyl^{b}, alkoxy, OH, halo, CN, and CF₃;
cycloalkyl is a monocyclic saturated hydrocarbon ring of between 3 and 6 carbon atoms (C₃-C₆); cycloalkyl may optionally be substituted with 1 or 2 substituents independently selected from alkyl, alkoxy, OH, CN, CF₃, halo;
halo is F, Cl, Br, or I;
heteroaryl is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃;
heteroaryl^{a} is a 5-, 6-, 9- or 10-membered mono- or bi-cyclic aromatic ring, containing, where possible, 1, 2, 3 or 4 ring members independently selected from N, N(R12), S and O; heteroaryl^{a} may be optionally substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃;
heteroaryl^{b} is a 5-, 6-, 9- or 10-membered mono- or bi-cyclic aromatic ring, containing, where possible, 1, 2 or 3 ring members independently selected from N, N(R12), S and O; wherein heteroaryl^{b} may be optionally substituted with 1, 2 or 3 substituents independently selected from alkyl^{b}, alkoxy, OH, halo, CN, aryl^{b}, —(CH₂)₁₋₃-aryl^{b}, and CF₃;
heterocycloalkyl is a non-aromatic carbon-containing monocyclic ring containing 3, 4, 5, or 6, ring members, wherein at least one ring member is independently selected from N, N(R12), S, and O; heterocycloalkyl may optionally be substituted with 1 or 2 substituents independently selected from alkyl, alkoxy, OH, CN, CF₃, halo;
heterocycloalkyl^{a} is a 3-10 membered non-aromatic carbon-containing mono- or bi-cyclic ring system containing one or two ring members independently selected from N and 0;
heterocycloalkyl^{a} may optionally be substituted with 1 or 2 substituents independently selected from alkyl, alkoxy, aryl, halo, OH, CN, CF₃, and heterocycloalkyl; wherein when heterocycloalkyl^{a} is a bicyclic ring system, the bicyclic ring system is spiro;
heterocycloalkyl^{b} is a 3-6 membered non-aromatic carbon-containing monocyclic ring system which may be saturated or, where possible, unsaturated with 1 or 2 double bonds;
heterocycloalkyl^{b} contains one or two ring members independently selected from N, N(C(═O)CH₃) and O; heterocycloalkyl^{b} may optionally be substituted with 1 or 2 substituents independently selected from alkyl, alkoxy, halo, OH, CN, CF₃, phenyl and oxo; wherein the point of attachment of heterocycloalkyl^{b} is not at a ring nitrogen;
polycycloalkyl is a bi- or tri-cyclic saturated hydrocarbon ring system of between 5 and 10 carbon atoms (C₅-C₁₀), wherein the bi- or tri-cyclic ring system is bridged or spiro;
R8 is independently selected from H, alkyl, cycloalkyl, and heterocycloalkyl;
R12 is independently selected from H, alkyl, and cycloalkyl;
R13 and R14 are independently selected from H, alkyl^{b}, aryl^{b} and heteroaryl^{b} or R13 and R14 together with the nitrogen atom to which they are attached form a carbon-containing 4-, 5-, 6- or 7-membered heterocyclic ring, optionally containing an additional heteroatom selected from N, N(R12), S, SO, SO₂, and O, which may be saturated or unsaturated with 1 or 2 double bonds and which may be optionally mono- or di-substituted with substituents selected from oxo, alkyl^{b}, alkoxy, OH, halo and CF₃;
R15 is selected from alkyl, halo, CF₃, CN, OH, alkoxy, NR13R14, and CONR13R14;
R16 and R17 are independently selected from H and small alkyl;
and tautomers, isomers, stereoisomers (including enantiomers, diastereoisomers and racemic and scalemic mixtures thereof), deuterated analogues, and pharmaceutically acceptable salts and/or solvates thereof.

2. A compound of formula (I) according to numbered embodiment 1, wherein the compound is a compound of formula (Ia), formula (Ib), or formula (Ic),

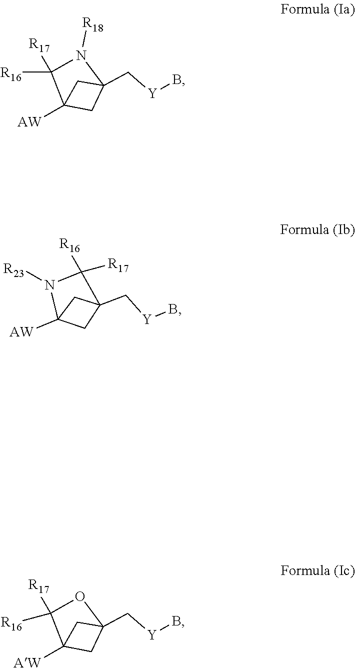

or a tautomer, isomer, stereoisomer (including an enantiomer, a diastereoisomer and a racemic and scalemic mixture thereof), a deuterated analogue, and a pharmaceutically acceptable salt and/or solvate thereof.

3. A compound according to numbered embodiment 1, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
V is —N(R23)-, and Z is selected from —C(R16)(R17)-CH₂— and —C(R16)(R17)-; or,
V is selected from —CH₂—C(R16)(R17)- and —C(R16)(R17)-, and Z is —N(R18)-.

4. A compound of formula (I) according to any of the preceding numbered embodiments, wherein the compound is a compound of formula (Ia) or formula (Ib),

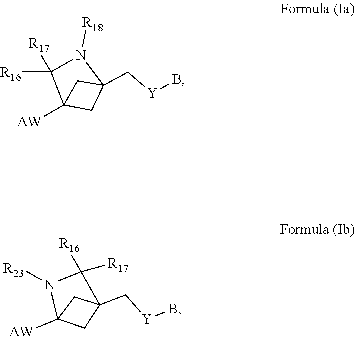

or a tautomer, isomer, stereoisomer (including an enantiomer, a diastereoisomer and a racemic and scalemic mixture thereof), a deuterated analogue, and a pharmaceutically acceptable salt and/or solvate thereof.

5. A compound of formula (I) according to any of the preceding numbered embodiments, wherein the compound is a compound of formula (Ia),

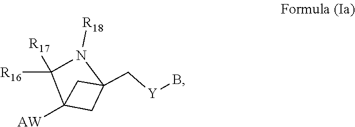

or a tautomer, isomer, stereoisomer (including an enantiomer, a diastereoisomer and a racemic and scalemic mixture thereof), a deuterated analogue, and a pharmaceutically acceptable salt and/or solvate thereof.

6. A compound according to numbered embodiment 3, 4 or 5, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
A is a 4- to 15-membered mono-, bi-, or tri-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, and CN;
wherein when A is a bicyclic ring system, the bicyclic ring system is fused, bridged or spiro;
wherein when A is a tricyclic ring system, each of the three rings in the tricyclic ring system is either fused, bridged or spiro to at least one of the other rings in the tricyclic ring system.

7. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R18 or R23 are selected from C(═S)NH-aryl; C(═S)NH—R19; C(═S)N(alkyl)R19; C(═S)NR13R14; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one substituent selected from cycloalkyl and heterocycloalkyl^{b}, and optionally one or two further substituents independently selected from alkyl, alkoxy, OH, OCF₃, halo, CN, and CF₃;

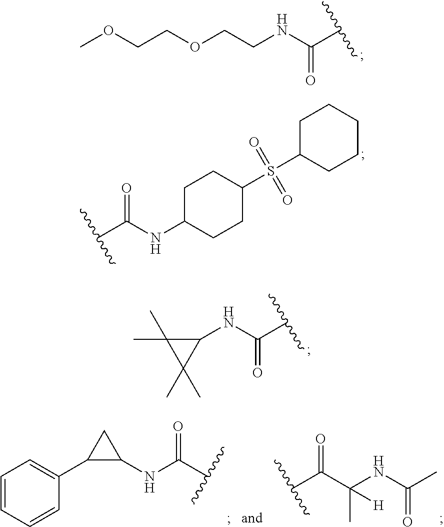

wherein R13 and R14 together with the nitrogen atom to which they are attached form a carbon-containing 4-, 5-, 6- or 7-membered heterocyclic ring, optionally containing an additional heteroatom selected from N, N(R12), S, SO, SO₂, and O, which may be saturated or unsaturated with 1 or 2 double bonds and which may be optionally mono- or di-substituted with substituents selected from oxo, alkyl^{b}, alkoxy, OH, halo and CF₃.

8. A compound according to any of numbered embodiments 1 to 6, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R18 or R23 are selected from C(═S)NH-aryl; C(═O)R19; C(═O)NHR19; C(═O)N(alkyl)(R19); C(═O)NH₂; heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

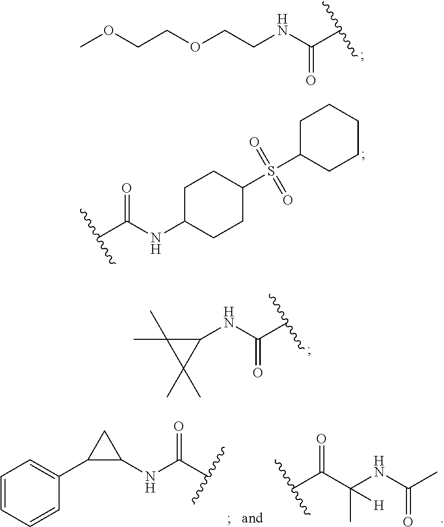

9. A compound according to numbered embodiment 8, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R18 or R23 are selected from C(═S)NH-phenyl; C(═O)R19; C(═O)NHR19; C(═O)N(alkyl)(R19); C(═O)NH₂, heterocycloalkyl^{b}; a 5- or 6-membered carbon-containing aromatic ring containing one, two or three ring members independently selected from N and, where possible, O, wherein the carbon-containing aromatic ring is substituted with one cycloalkyl substituent;

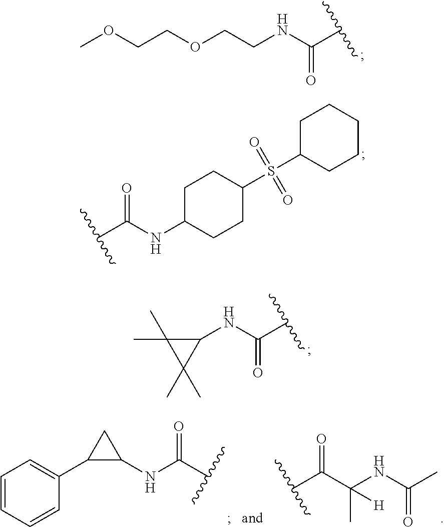

10. A compound according to numbered embodiment 9, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R18 or R23 are selected from C(═S)NH-phenyl; C(═O)R19; C(═O)NHR19; C(═O)N(alkyl)(R19): C(═O)NH₂; heterocycloalkyl^{b}; a 6-membered carbon-containing aromatic ring containing one or two N ring members substituted with one cycloalkyl substituent;

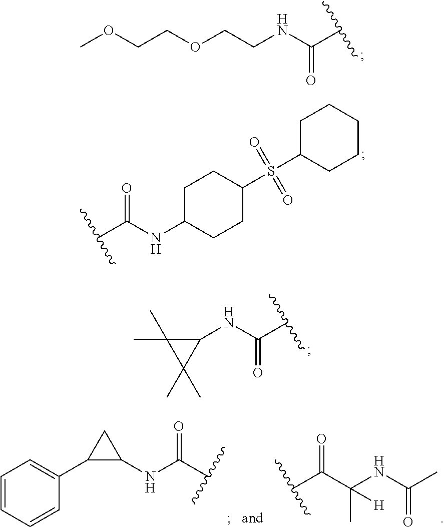

11. A compound according to numbered embodiment 10, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R18 or R23 are selected from C(═S)NH-phenyl; C(═O)R19; C(═O)NHR19; C(═O)N(alkyl)(R19); C(═O)NH₂; 5- or 6-membered heterocycloalkyl^{b} ring, optionally substituted as for heterocycloalkyl^{b}; a 6-membered carbon-containing aromatic ring containing one or two N ring members substituted with one cycloalkyl substituent;

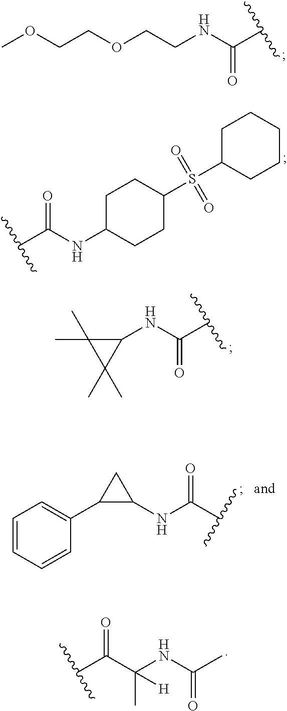

12. A compound according to numbered embodiment 11, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R18 or R23 are selected from C(═S)NH-phenyl; C(═O)R19; C(═O)NHR19; C(═O)N(alkyl)(R19); C(═O)NH₂; 5-membered heterocycloalkyl^{b} ring, optionally substituted as for heterocycloalkyl^{b}; a 6-membered carbon-containing aromatic ring containing one or two N ring members substituted with one cycloalkyl substituent:

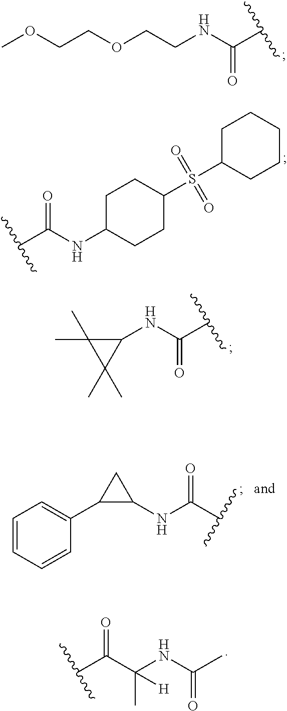

13. A compound according to numbered embodiment 12, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R18 or R23 are selected from C(═S)NH-phenyl; C(═O)R19; C(═O)NHR19; C(═O)N(alkyl)(R19); C(═O)NH₂; 5-membered heterocycloalkyl^{b} ring, optionally substituted as for heterocycloalkyl^{b}; a 6-membered carbon-containing aromatic ring containing one or two N ring members substituted with one

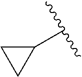 [SMILES: *C1CC1]

substituent;

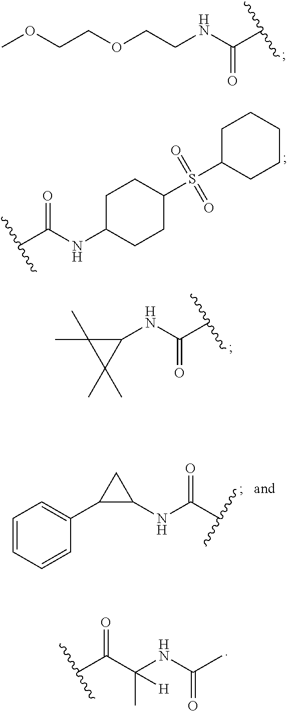

14. A compound according numbered embodiment 13, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R18 or R23 are selected from C(═S)NH-phenyl; C(═O)R19; C(═O)NHR19; C(═O)N(alkyl)(R19); C(═O)NH₂; 5-membered heterocycloalkyl^{b} ring, optionally substituted as for heterocycloalkyl^{b}; a 6-membered carbon-containing aromatic ring containing two N ring members substituted with one

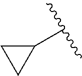 [SMILES: *C1CC1]

substituent;

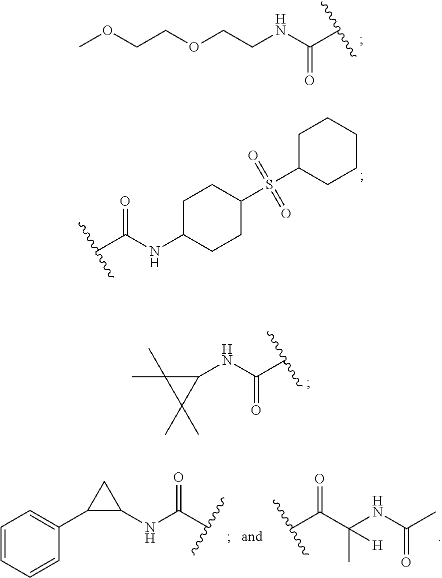

15. A compound according to any of the preceding numbered embodiments or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Ia), wherein R18 is as described in any of the preceding numbered embodiments, or a compound of formula (Ib) and R23 is C(═O)R19.

16. A compound according to numbered embodiment 15, or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Ia), wherein R18 is as described in any of the preceding numbered embodiments, or a compound of formula (Ib) and R23 is C(═O)(CH₂)₁₋₃cycloalkyl.

17. A compound according to numbered embodiment 16, or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Ia), wherein R18 is as described in any of the preceding numbered embodiments, or a compound of formula (Ib) and R23 is C(═O)(CH₂)₁₋₂cycloalkyl.

18. A compound according to numbered embodiment 17, or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Ia), wherein R18 is as described in any of the preceding numbered embodiments, or a compound of formula (Ib) and R23 is selected from

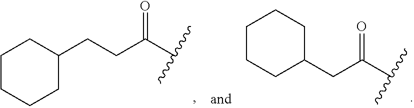 [SMILES: *C(=O)CCC1CCCCC1 ; *C(=O)CC1CCCCC1]

19. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R16 is H.

20. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R16 and R17 are both H.

21. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, (C(R20)(R21))-O-phenyl, and (CH₂)₃—O—(CH₂)-phenyl.

22. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R19 is selected from (CH₂)₁₋₂-cycloalkyl, (CH₂)₀₋₁-polycycloalkyl, heterocycloalkyl^{a}, (CH₂)₁₋₂-heterocycloalkyl^{b}, (CH₂)₀₋₂—R22, (C(R20)(R21))-(CH₂)₀₋₁-phenyl, (C(R20)(R21))-R22, and (CH₂)₃—O—(CH₂)-phenyl.

23. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein heterocycloalkyl^{a} is a 4-10 membered non-aromatic carbon-containing mono- or bi-cyclic ring system containing one or two ring members independently selected from N and O; heterocycloalkyl^{a} may optionally be substituted with 1 or 2 substituents independently selected from alkyl, aryl, halo, and heterocycloalkyl; wherein when heterocycloalkyl^{a} is a bicyclic ring system, the bicyclic ring system is spiro.

24. A compound according to numbered embodiment 23, or a tautomer, isomer, stereoisomer (including an enantiomer, a diastereoisomer and a racemic and scalemic mixture thereof), a deuterated analogue, and a pharmaceutically acceptable salt and/or solvate thereof, wherein heterocycloalkyl^{a} is selected from:

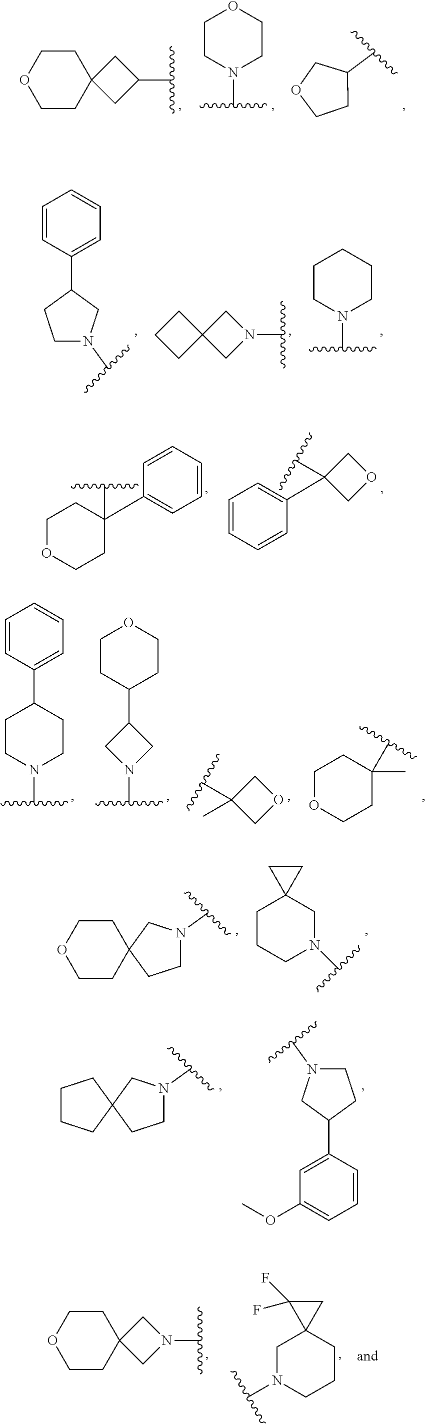

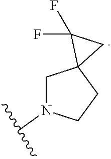 [SMILES: *N1CCC2(C1)CC2(F)F]

25. A compound according to numbered embodiment 24, or a tautomer, isomer, stereoisomer (including an enantiomer, a diastereoisomer and a racemic and scalemic mixture thereof), a deuterated analogue, and a pharmaceutically acceptable salt and/or solvate thereof, wherein heterocycloalkyl^{a} is selected from:

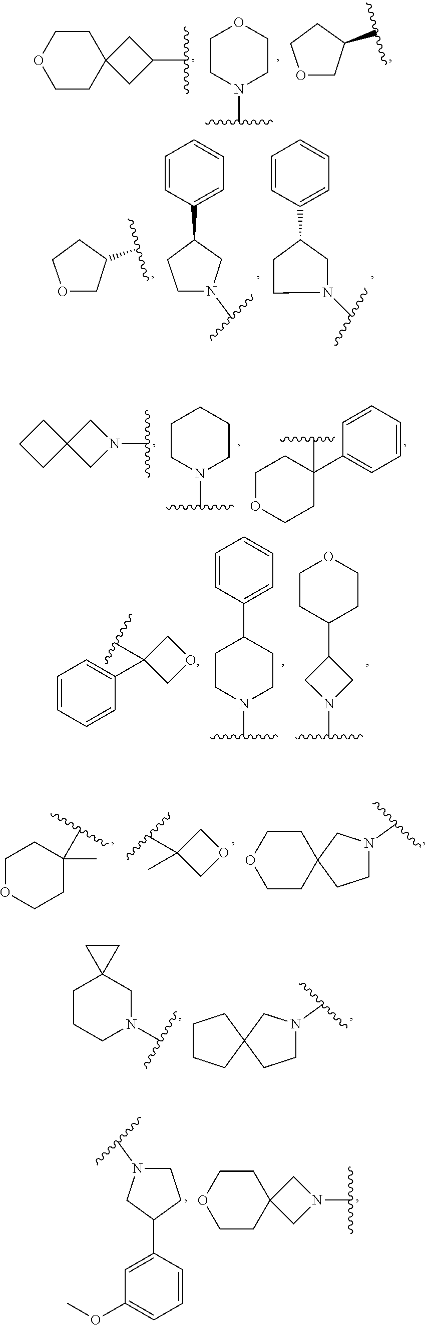

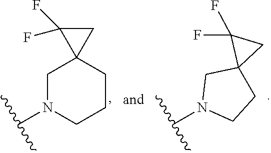 [SMILES: *N1CCCC2(C1)CC2(F)F ; *N1CCC2(C1)CC2(F)F]

26. A compound according to any of numbered embodiments 1 to 22, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
heterocycloalkyl^{b} is a 4-6 membered non-aromatic carbon-containing monocyclic ring system which may be saturated or, where possible, unsaturated with 1 or 2 double bonds; heterocycloalkyl^{b} contains one or two ring members independently selected from N, N(C(═O)CH₃) and O; heterocycloalkyl^{b} may optionally be substituted with 1 or 2 substituents independently selected from alkyl, phenyl and oxo; wherein the point of attachment of heterocycloalkyl^{b} is not at a ring nitrogen.

27. A compound according to numbered embodiment 26, or a tautomer, isomer, stereoisomer (including an enantiomer, a diastereoisomer and a racemic and scalemic mixture thereof), a deuterated analogue, and a pharmaceutically acceptable salt and/or solvate thereof, wherein heterocycloalkyl^{b} is selected from:

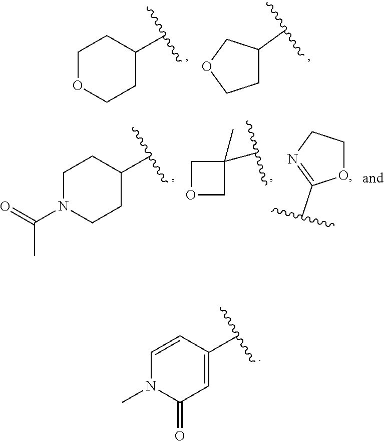

28. A compound according to numbered embodiment 27, or a tautomer, isomer, stereoisomer (including an enantiomer, a diastereoisomer and a racemic and scalemic mixture thereof), a deuterated analogue, and a pharmaceutically acceptable salt and/or solvate thereof, wherein heterocycloalkyl^{b} is selected from:

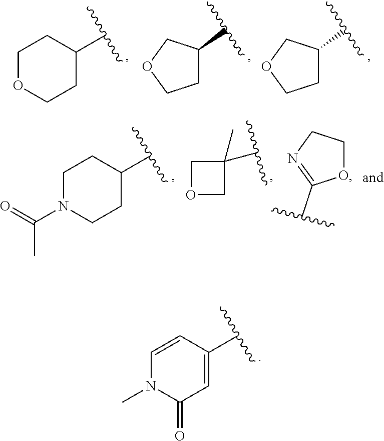

29. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein polycycloalkyl is selected from:

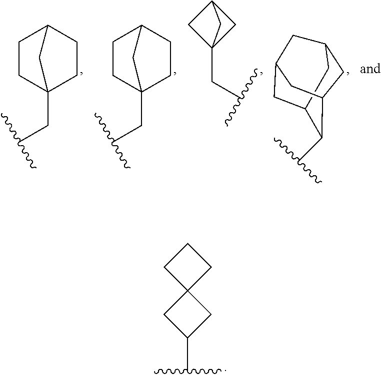

30. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R22 is a fused 6,5- or 6,6-bicyclic ring containing an aromatic ring fused to a non-aromatic ring, wherein the bicyclic ring optionally contains one or two ring members selected from N and O, wherein the fused 6,5- or 6,6-bicyclic ring may be optionally substituted with one or two substituents independently selected from alkoxy, halo, and oxo, wherein the 6,5-bicyclic ring may be attached via the 6- or 5-membered ring, wherein the 6,5-bicyclic ring is not aryl substituted with methylenedioxy, and wherein the 6,6-bicyclic ring is not aryl substituted with ethylenedioxy.

31. A compound according to numbered embodiment 30, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R22 is a fused 6,5- or 6,6-bicyclic ring containing a 6-membered aromatic ring fused to a non-aromatic ring, wherein the bicyclic ring optionally contains one or two ring members selected from N and O, wherein the fused 6,5- or 6,6-bicyclic ring may be optionally substituted with one or two substituents independently selected from alkoxy, halo, and oxo, wherein the 6,5-bicyclic ring may be attached via the 6- or 5-membered ring, wherein the 6,5-bicyclic ring is not aryl substituted with methylenedioxy, and wherein the 6,6-bicyclic ring is not aryl substituted with ethylenedioxy.

32. A compound according to numbered embodiment 31, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R22 is selected from;

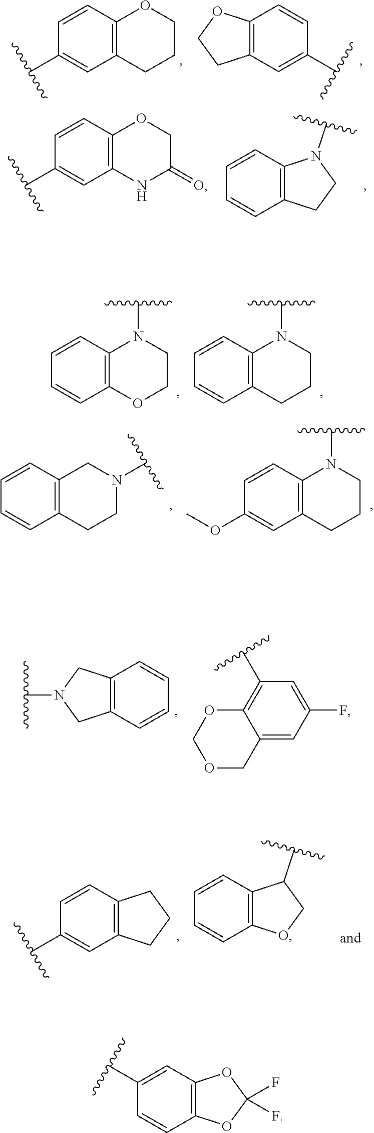

33. A compound according to numbered embodiment 31, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R22 is selected from:

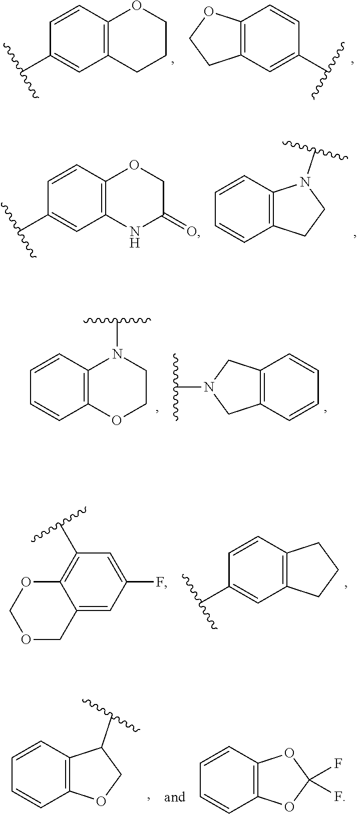

34. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R20 and R21 together with the carbon atom to which they are attached form a carbon-containing 3-, 4- or 6-membered saturated ring, optionally containing an O ring member, wherein R20 and R21 are not both H.

35. A compound according to any of numbered embodiments 1 to 33, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R20 or R21 are independently selected from H, (C₁₋₃)alkyl and CF₃, wherein R20 and R21 are not both H.

36. A compound according to numbered embodiment 35, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R20 or R21 are independently selected from H, methyl and CF₃, wherein R20 and R21 are not both H.

37. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R18 can be selected from:

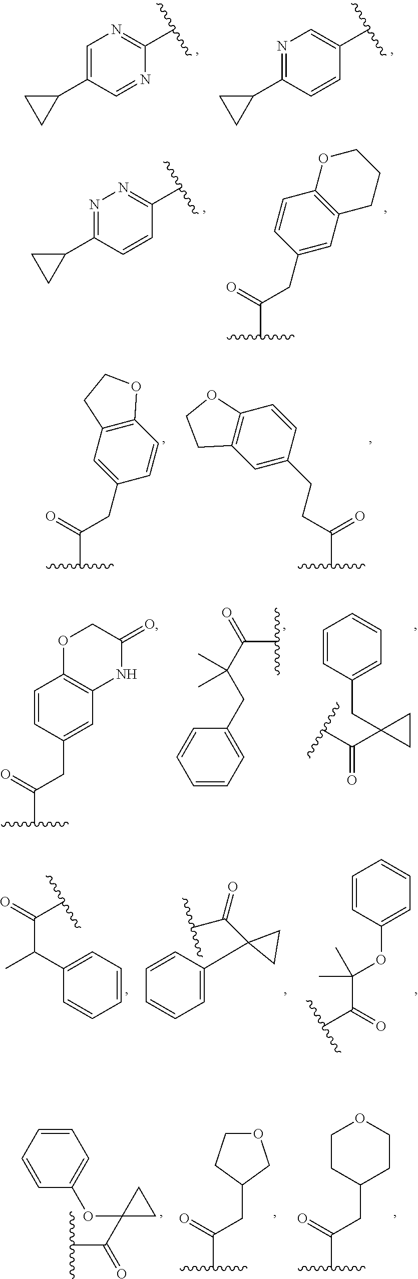

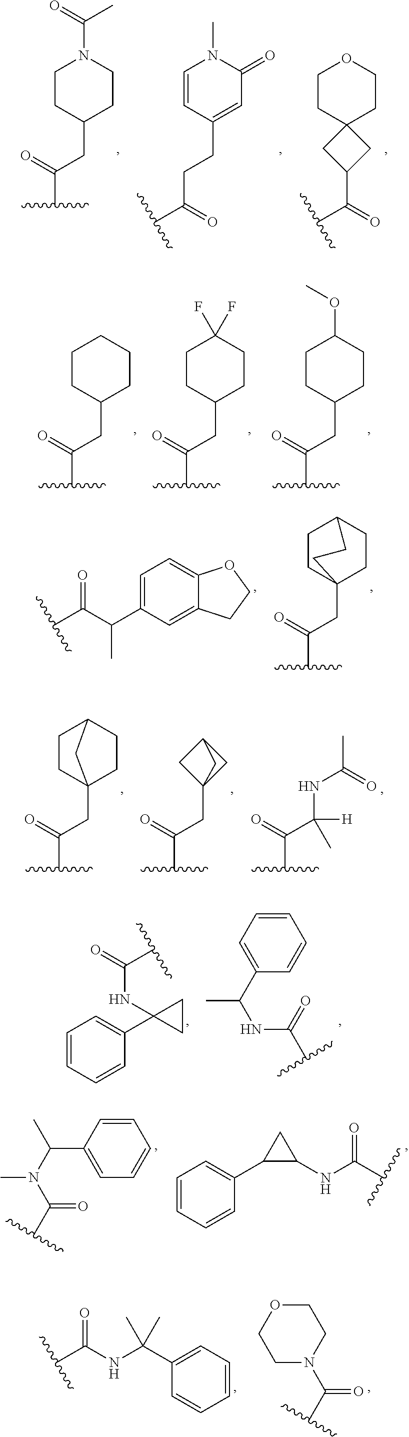

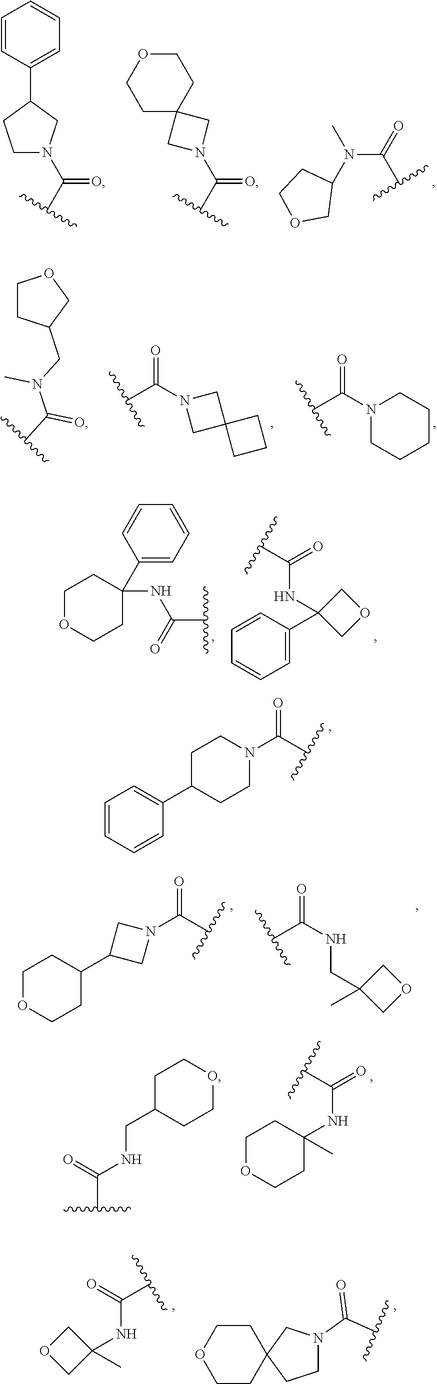

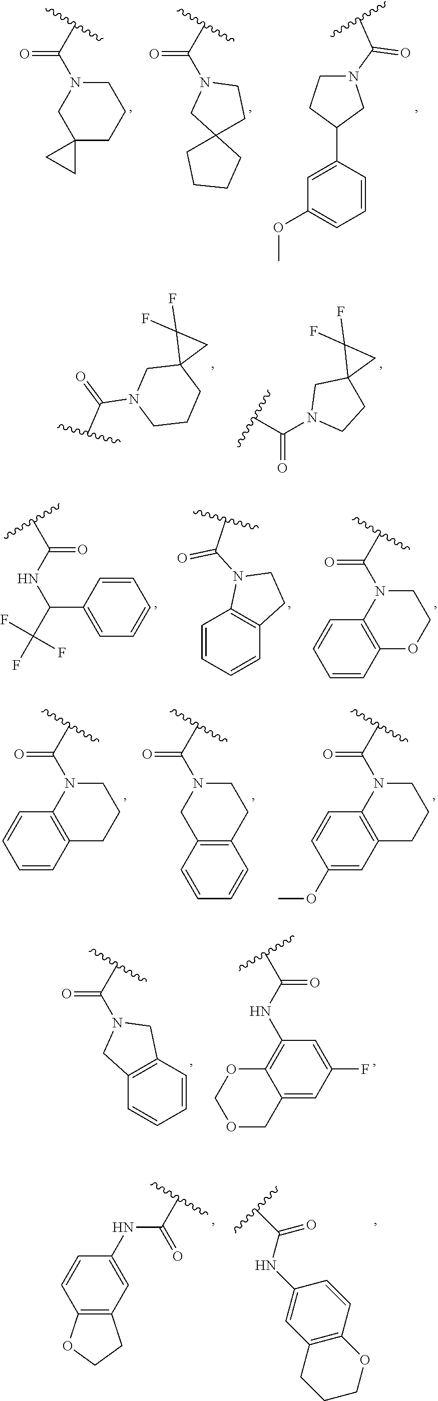

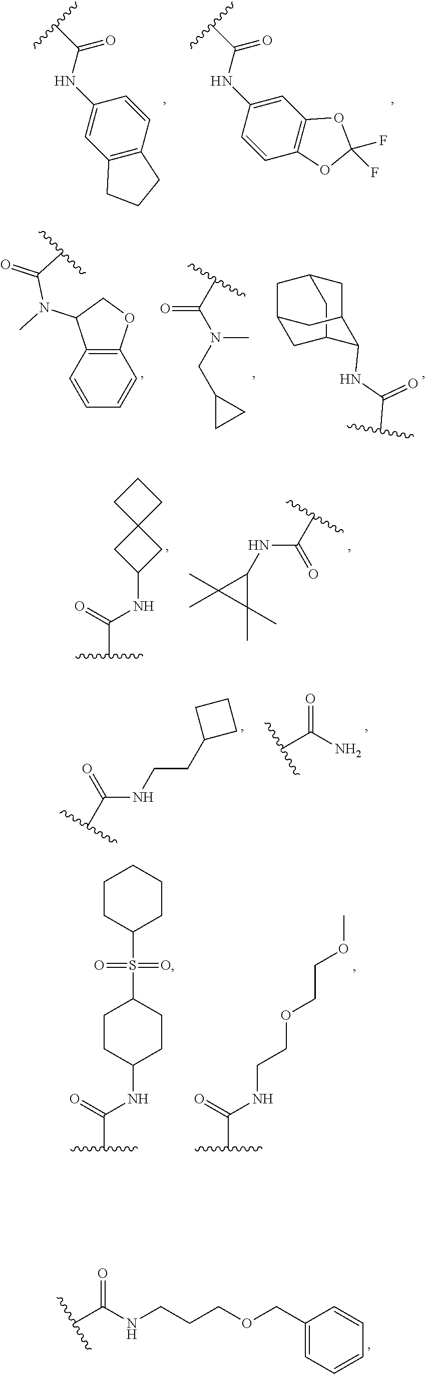

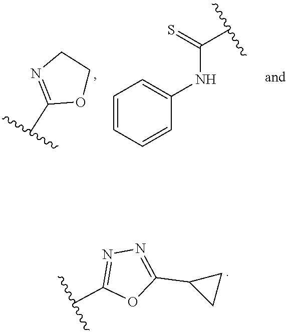

38. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R18 can be selected from:

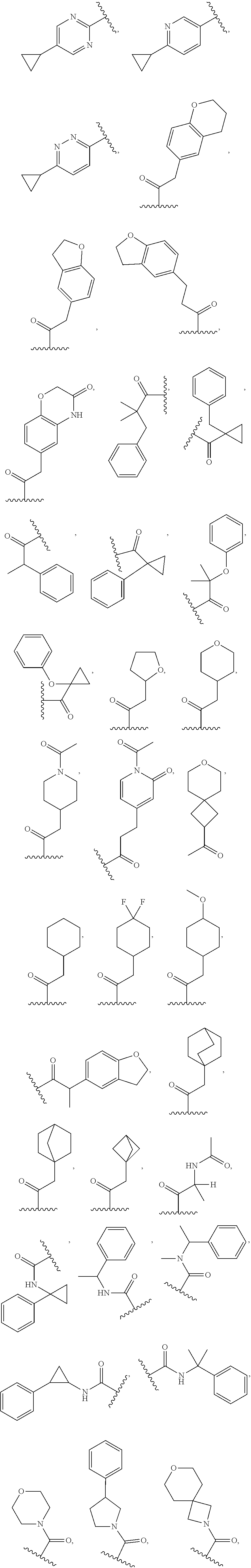

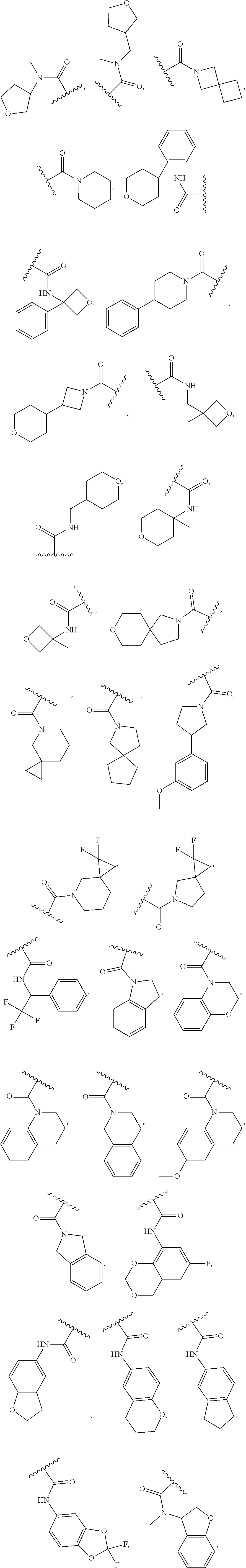

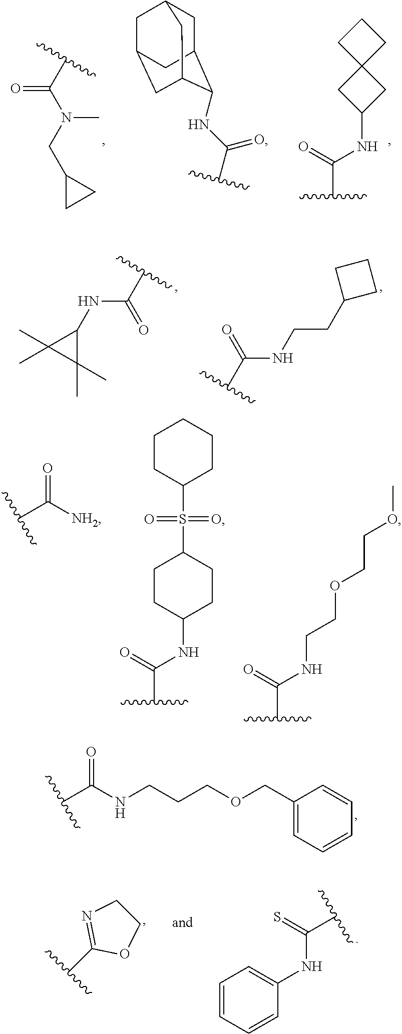

39. A compound according to numbered embodiment 37, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R18 is selected from:

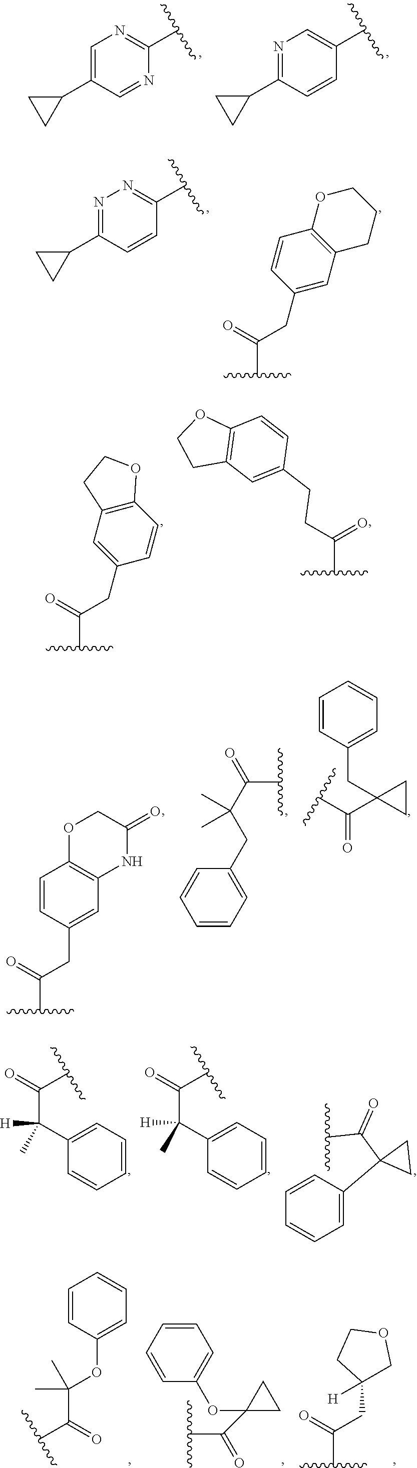

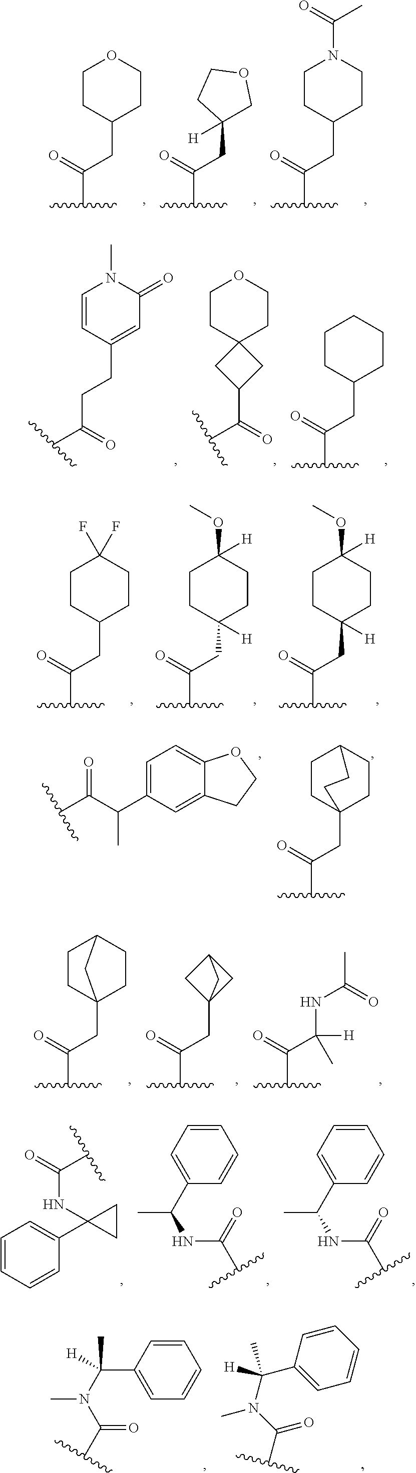

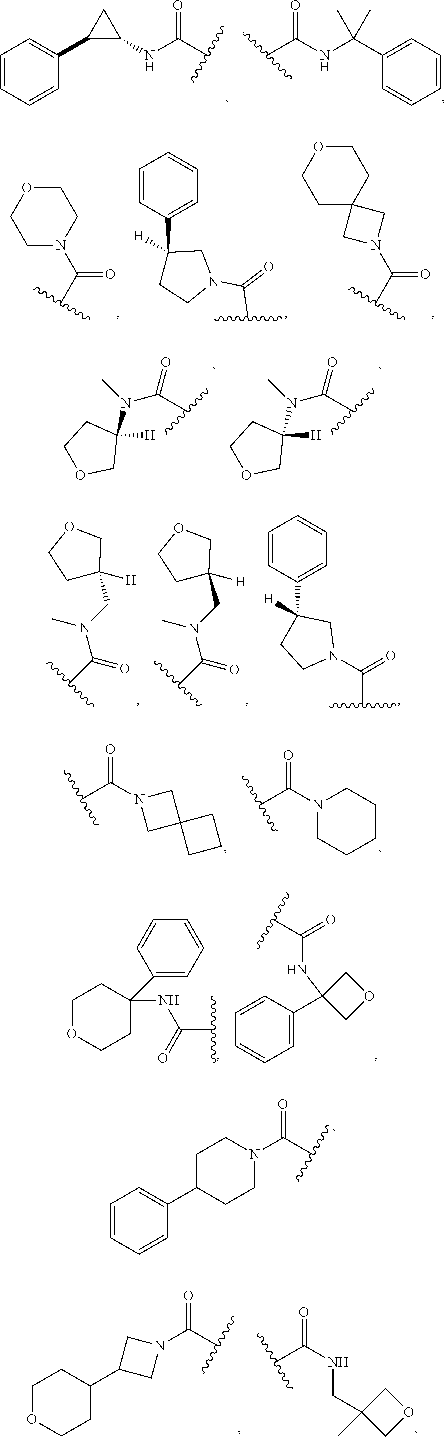

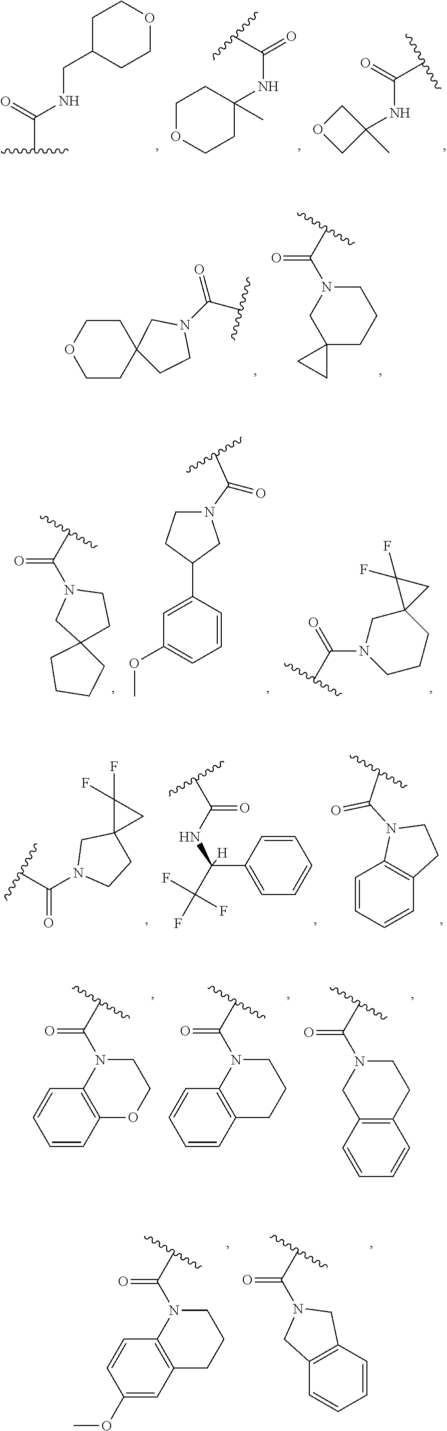

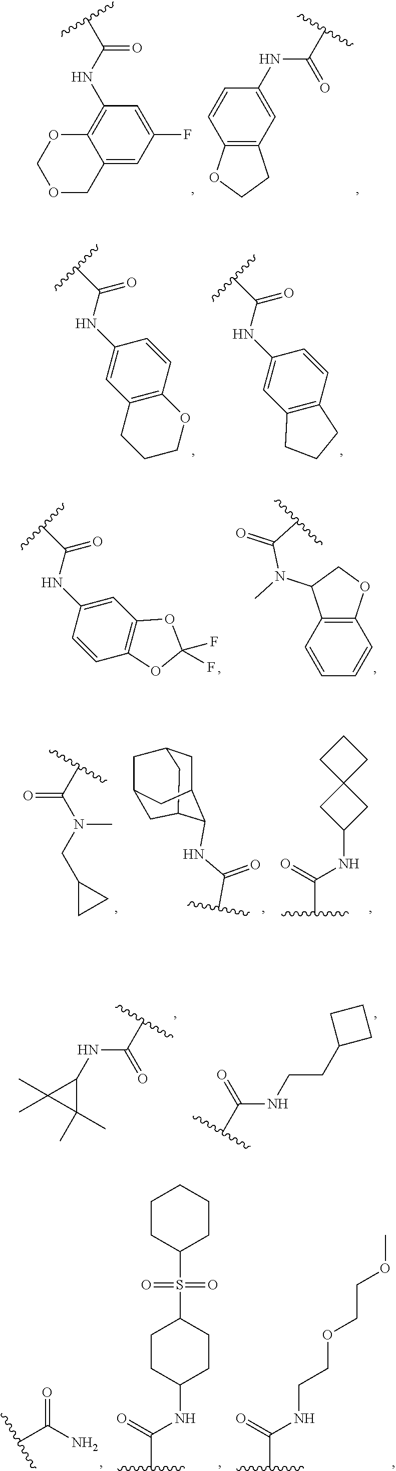

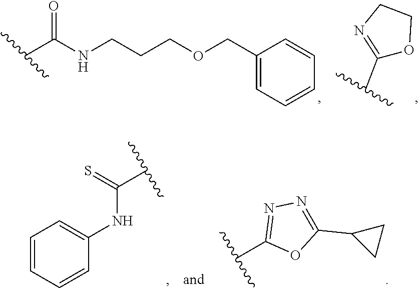 [SMILES: *C(=O)NCCCOCc1ccccc1 ; *C1=NCCO1 ; *C(=S)Nc1ccccc1 ; *c1nnc(C2CC2)o1]

40. A compound according to numbered embodiment 39, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R18 is selected from:

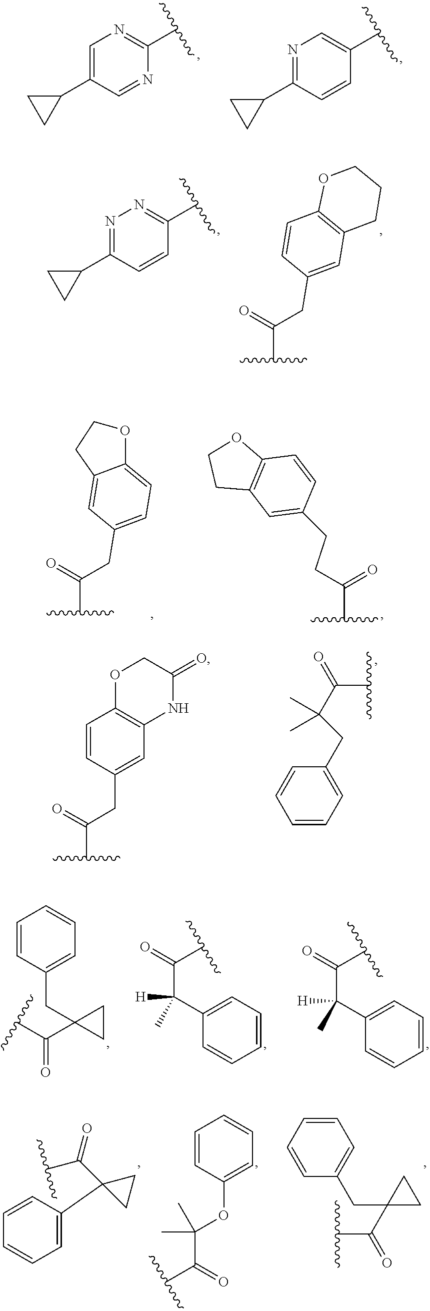

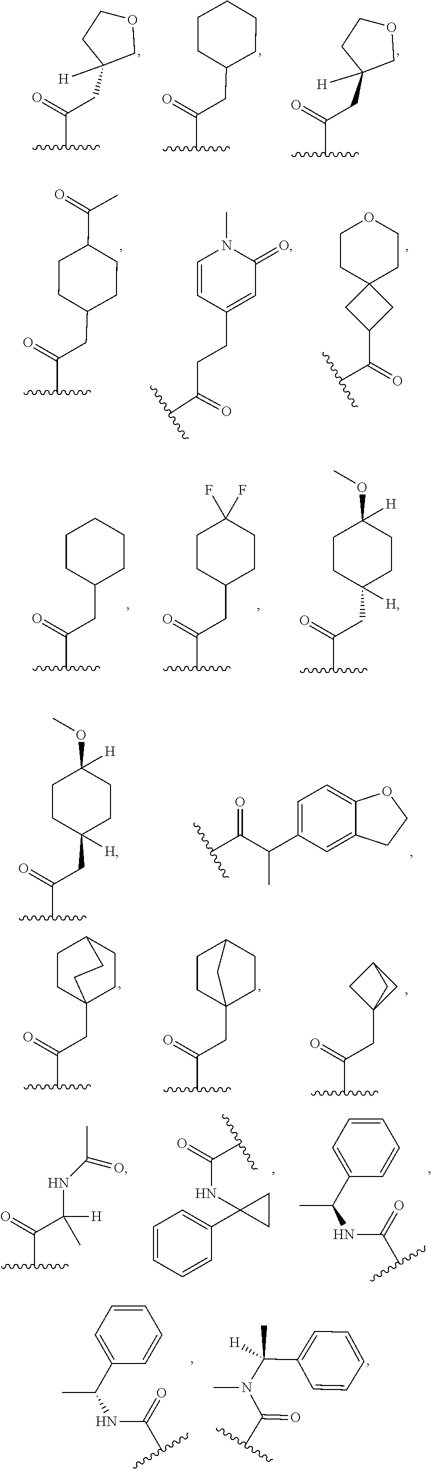

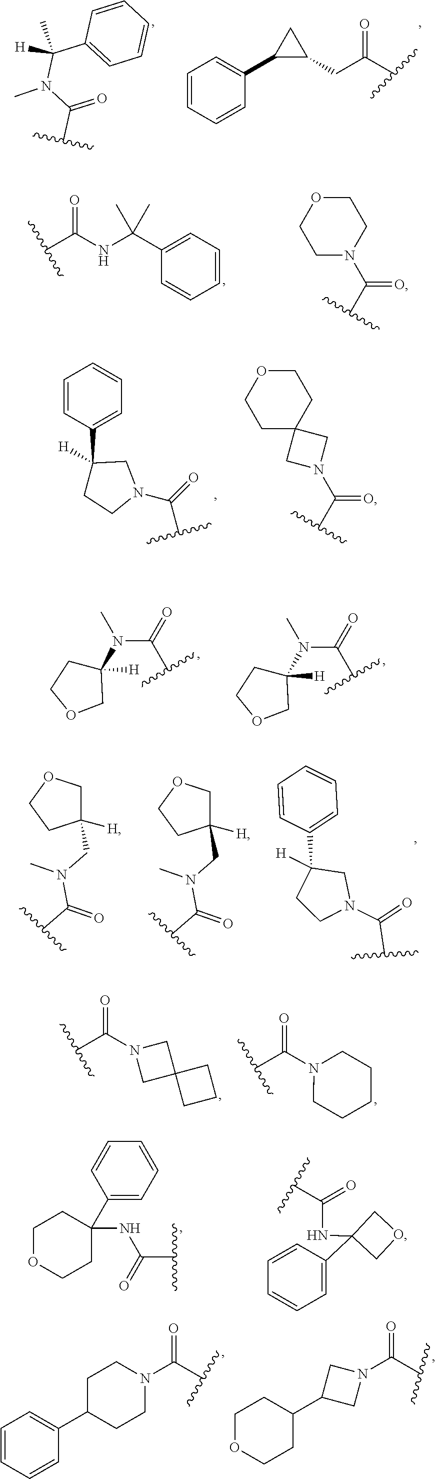

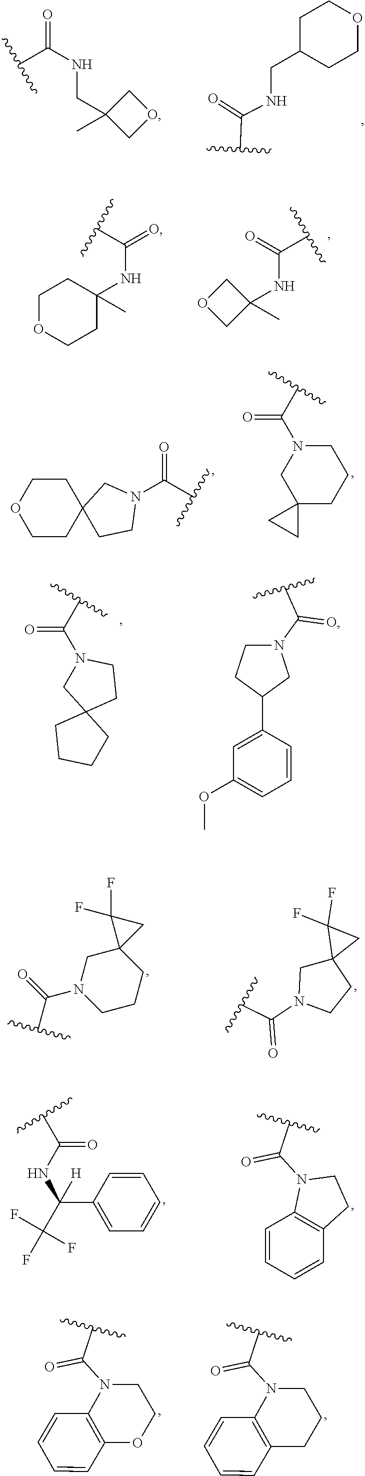

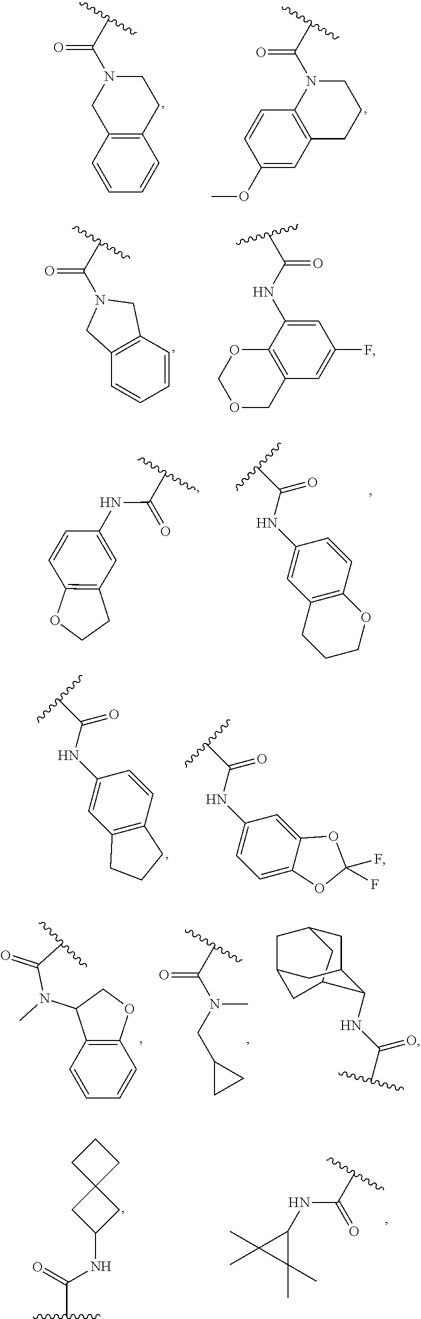

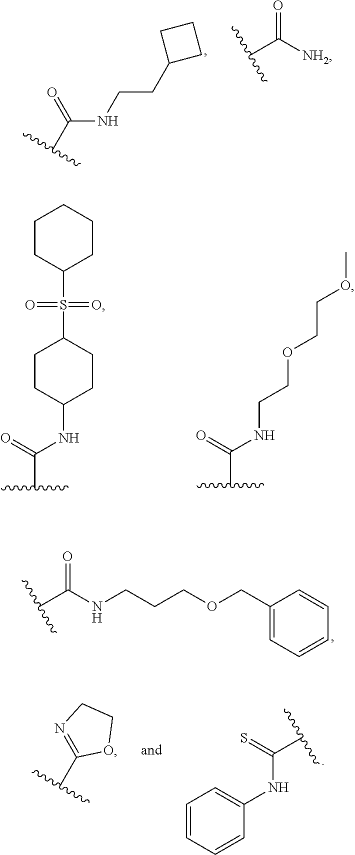

41. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R18 is selected from:

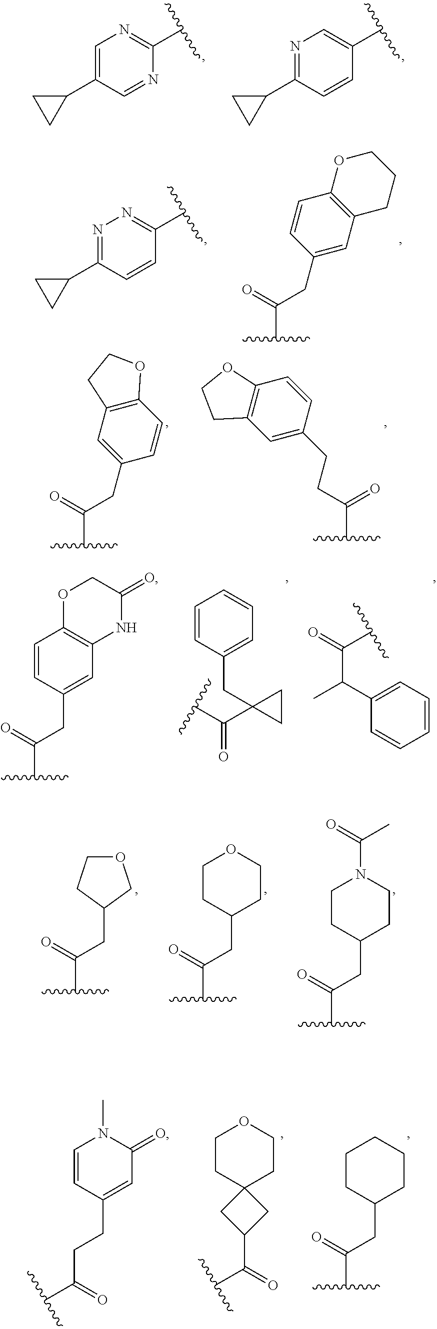

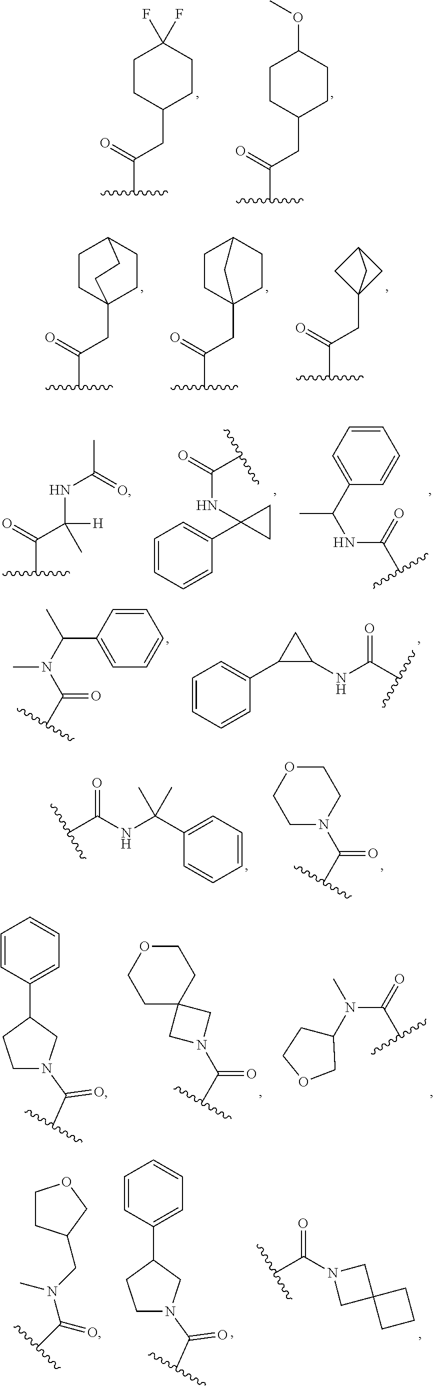

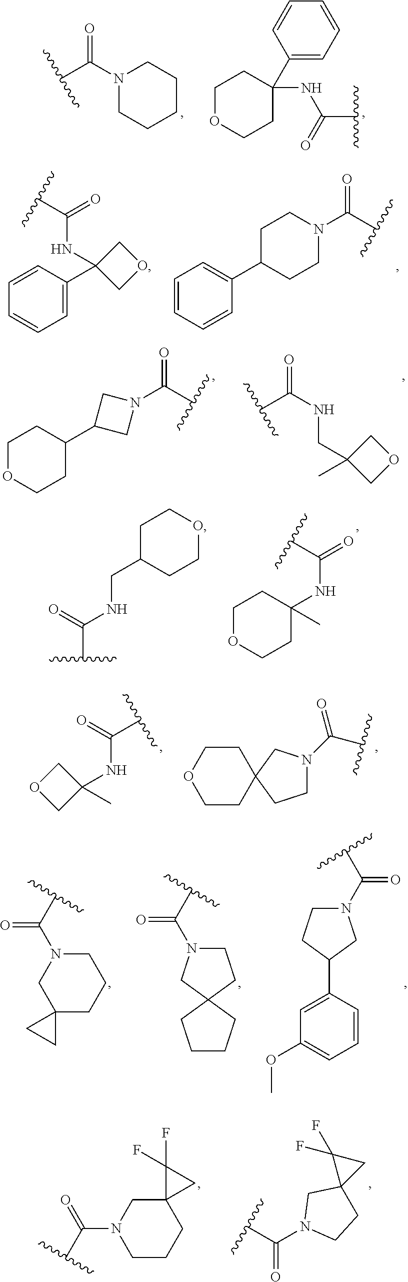

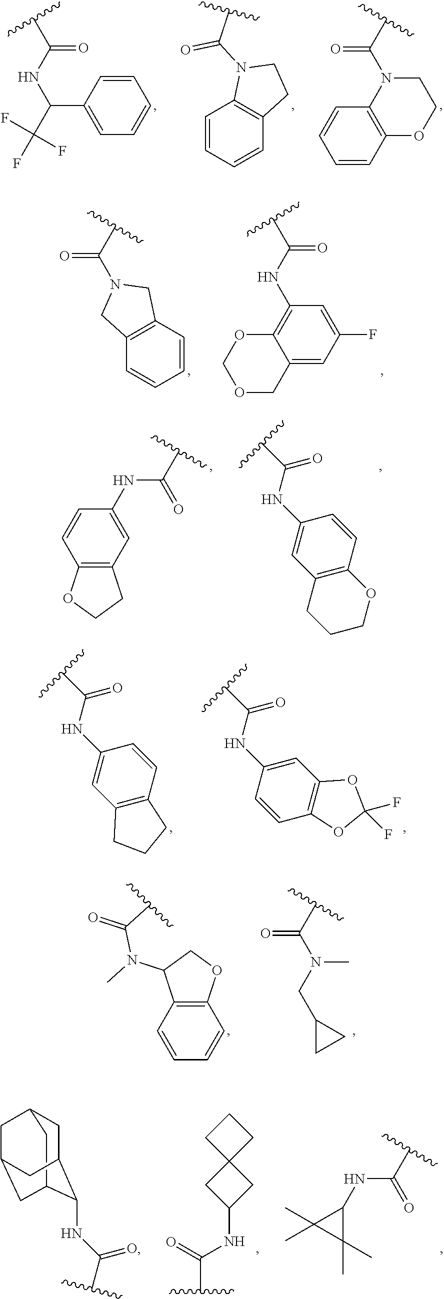

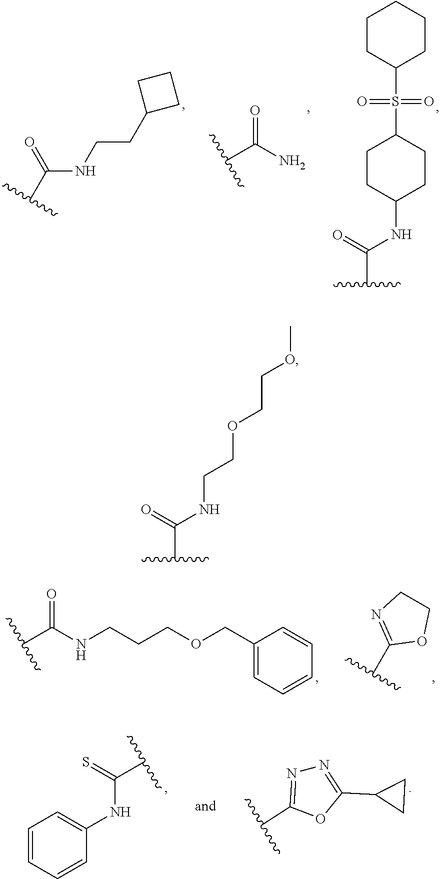

42. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R-18 is selected from:

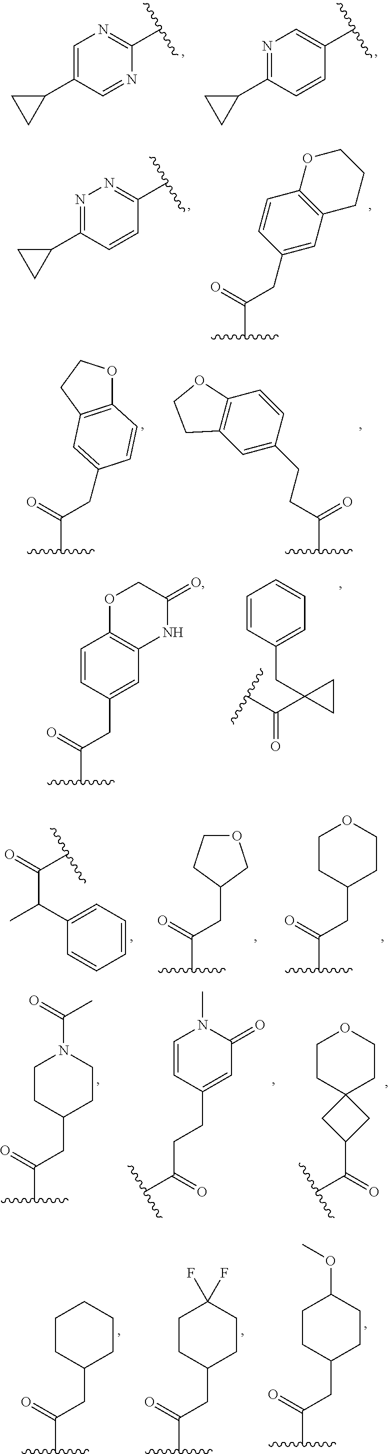

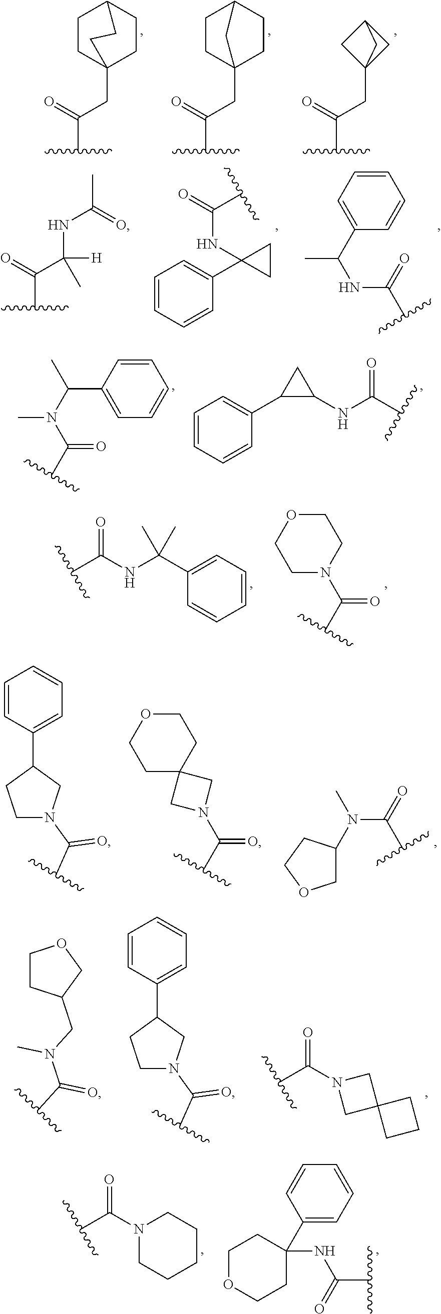

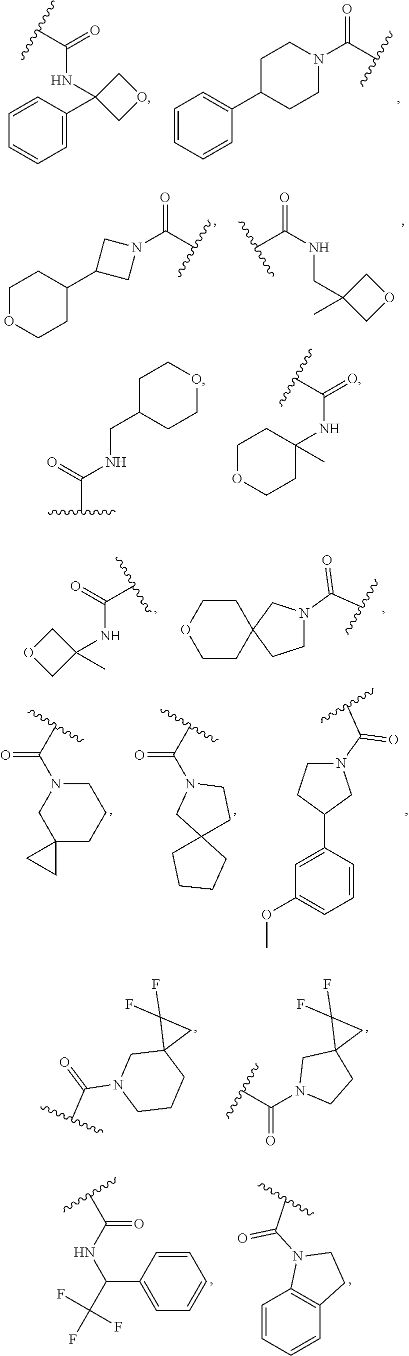

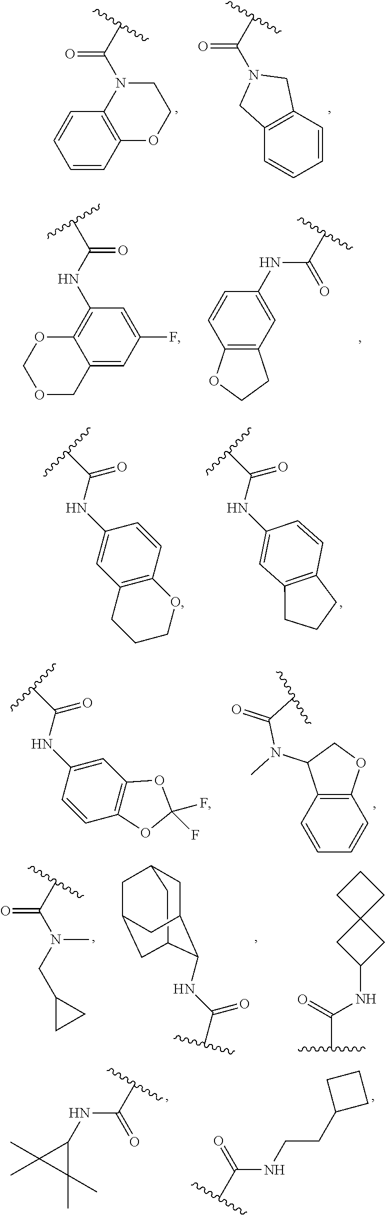

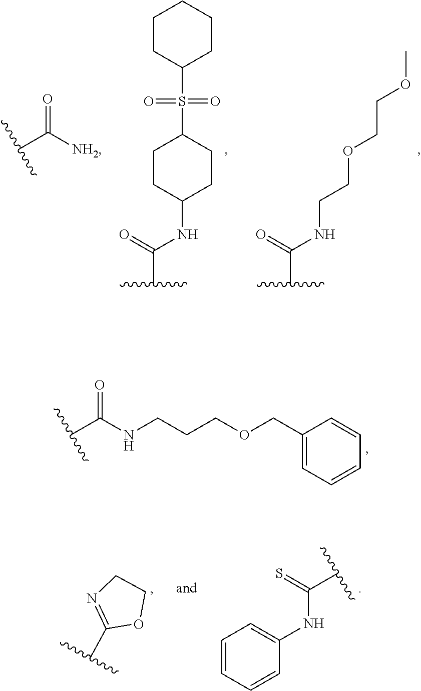

43. A compound according to numbered embodiment 41, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R18 is selected from:

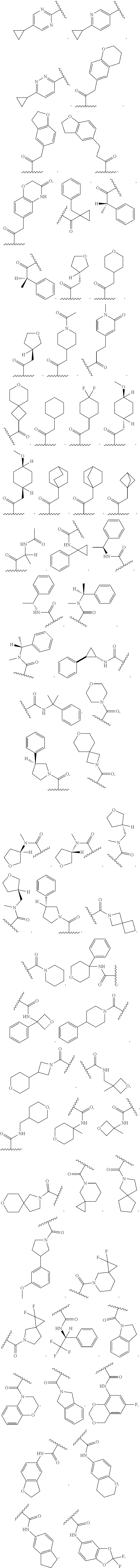

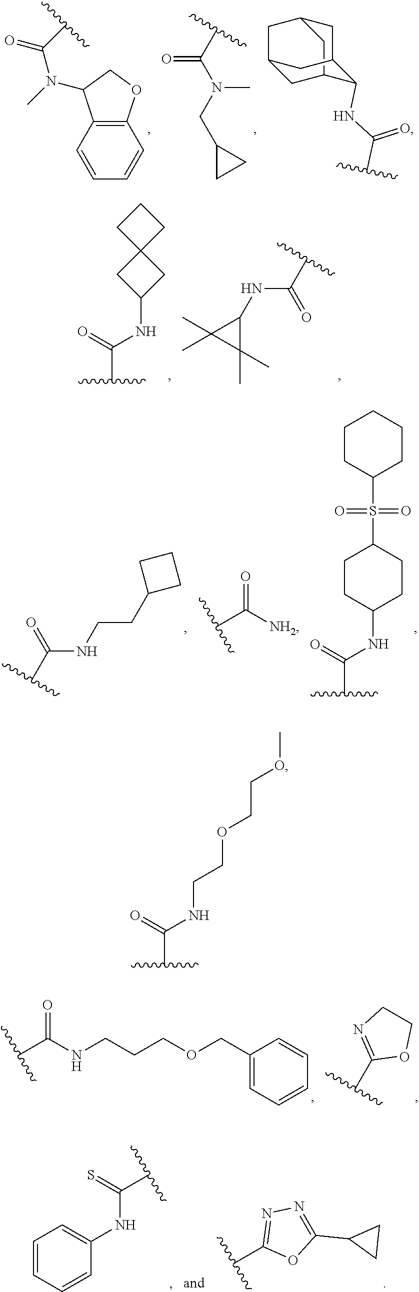

44. A compound according to numbered embodiment 43, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R18 is selected from:

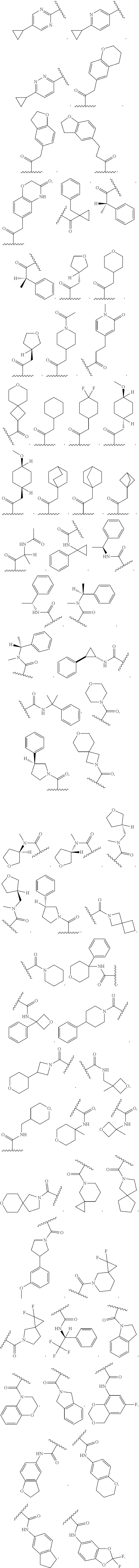

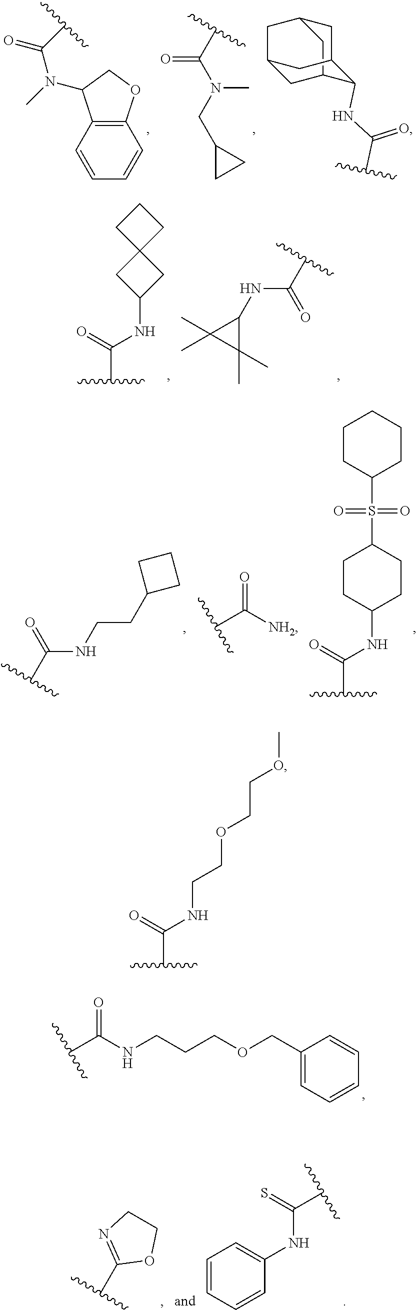

45. A compound according to numbered embodiment 44, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R18 is selected from:

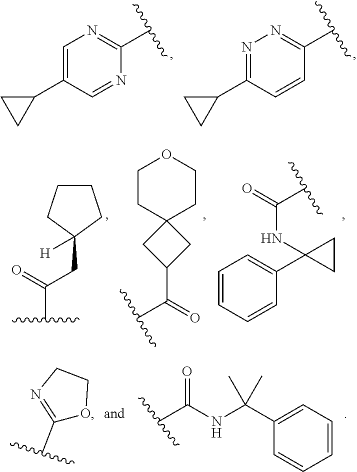

46. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is a 6 to 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein when A is a bicyclic ring system, the bicyclic ring system is fused, bridged or spiro.

47. A compound according to numbered embodiment 46, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
A is a 6 or 9 membered mono or bi-cyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein when A is a bicyclic ring system, the bicyclic ring system is fused.

48. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
A is substituted, where possible, with 1, 2 or 3 substituents independently selected from alkyl and oxo.

49. A compound according to numbered embodiments 46 to 48, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
A is selected from:

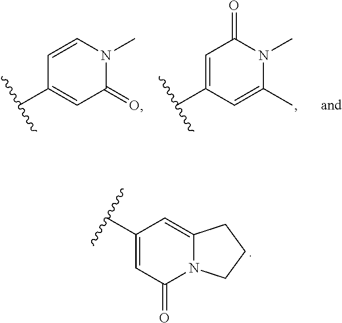

50. A compound according to numbered embodiment 1, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
V is —O—, and Z is selected from —C(R16)(R17)-CH₂— and —C(R16)(R17)-; or,
V is selected from —CH₂—C(R16)(R17)- and —C(R16)(R17)-, and Z is —O—.

51. A compound of formula (I) according to numbered embodiment 1, 2 or 50, wherein the compound is a compound of formula (Ic),

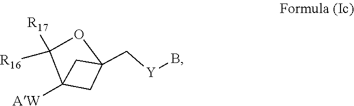

or a tautomer, isomer, stereoisomer (including an enantiomer, a diastereoisomer and a racemic and scalemic mixture thereof), a deuterated analogue, and a pharmaceutically acceptable salt and/or solvate thereof.

52. A compound according to numbered embodiment 50 or 51, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R16 is H.

53. A compound according to any of numbered embodiments 50 to 52, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
R16 and R17 are both H.

54. A compound according to any of numbered embodiments 1, 2 or 50 to 53, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
A′ is a 5-membered monocyclic ring system containing one N ring member and optionally one further ring member selected from N and O, wherein the ring system is substituted with phenyl and optionally, where possible, 1, 2, or 3 further substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN, and phenyl.

55. A compound of formula (I) according to numbered embodiment 54 or a tautomer, isomer, stereoisomer (including an enantiomer, a diastereoisomer and a racemic and scalemic mixture thereof), a deuterated isotope, and a pharmaceutically acceptable salt and/or solvate thereof, wherein
A′ is a 5-membered monocyclic ring system containing one N ring member and optionally one further ring member selected from N and O, wherein the ring system is substituted with phenyl.

56. A compound of formula (I) according to numbered embodiment 55 or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is selected from

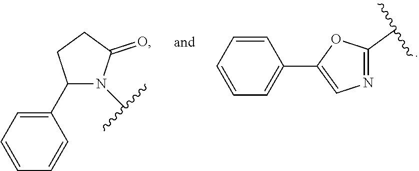 [SMILES: *N1C(=O)CCC1c1ccccc1 ; "and" ; *c1ncc(-c2ccccc2)o1]

57. A compound of formula (I) according to numbered embodiment 56 or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is selected from:

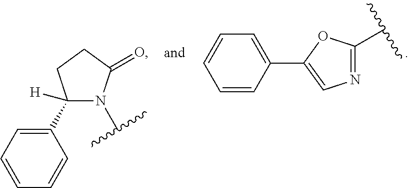 [SMILES: *N1C(=O)CC[C@H]1c1ccccc1 ; *c1ncc(-c2ccccc2)o1]

58. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
AW— is selected from —(CH₂)₀₋₃—(CHR15)-(CH₂)₀₋₃-A, —(CHR12)-A, —O—(CHR12)-A, —(CH₂)₀₋₃-A, —(CH₂)₀₋₃—O—(CH₂)₀₋₃-A, —(CH₂)₀₋₃—NH—(CH₂)₀₋₃-A, —(CH₂)₀₋₃—N(R12)-(CH₂)₁₋₃—C(═O)-A, —(CH₂)₀₋₃—NH—C(═O)—(CH₂)₀₋₃-A, —C(═O)N(R12)-(CH₂)₀₋₃-A, —(CH₂)₀₋₃—C(═O)—(CH₂)₀₋₃-A, —(CH₂)₀₋₃-(phenyl)-(CH₂)₀₋₃-A, —NH—SO₂-A and —SO₂—NH-A.

59. A compound according to numbered embodiment 58, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
AW— is selected from —(CH₂)₀₋₃-A, and —(CH₂)₀₋₃—O—(CH₂)₀₋₃-A.

60. A compound according to numbered embodiment 59, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
AW— is selected from —(CH₂)₀₋₁-A, and —(CH₂)₀₋₁O-A

61. A compound according to numbered embodiment 60, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
AW— is selected from —(CH₂)₀₋₁-A, and —(CH₂)—O-A.

62. A compound according to any of numbered embodiments 1 to 49, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
AW— is —(CH₂)O₁—O-A.

63. A compound according to numbered embodiment 62, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
AW— is —(CH₂)—O-A.

64. A compound according to any of numbered embodiments 1, 2, or 50 to 57, or a tautomer, isomer, stereoisomer (including an enantiomer, a diastereoisomer and a racemic and scalemic mixture thereof), a deuterated analogue, and a pharmaceutically acceptable salt and/or solvate thereof, wherein AW— is A′W— and A’W— is selected from —(CH₂)₀₋₃-A′.

65. A compound according to numbered embodiment 64, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
A′W— is —(CH₂)₀₋₁-A′.

66. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein

Y is —N(R12)-.

67. A compound according to numbered embodiment 66, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
Y is —NH— or N(CH₃).

68. A compound according to any one of numbered embodiments 66 or 67, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
Y is —NH—.

69. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
B is aryl; or B is a 6-membered monocyclic or 9- or 10-membered bicyclic aromatic ring, containing, where possible, 1, 2, 3 or 4 ring members independently selected from N, N(R12), S and O, optionally substituted as for heteroaryl^{a}.

70. A compound according to numbered embodiment 69, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is aryl; or B is a 6-membered monocyclic or 10-membered bicyclic aromatic ring, containing, where possible, 1, 2, 3 or 4 ring members independently selected from N, N(R12), S and O, optionally substituted as for heteroaryl^{a}.

71. A compound according to numbered embodiment 70, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is aryl; or B is a 6-membered monocyclic or 10-membered bicyclic aromatic ring, containing 1 or 2 ring members independently selected from N or N(R12), optionally substituted as for heteroaryl^{a}.

72. A compound according to numbered embodiment 71, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
B is phenyl substituted as for aryl; or B is a 6-membered monocyclic or 9- or 10-membered bicyclic aromatic ring, containing, where possible, 1, 2, 3 or 4 ring members independently selected from N, N(R12), S and O, optionally substituted as for heteroaryl^{a}.

73. A compound according to numbered embodiment 72, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
B is phenyl substituted as for aryl; or B is a 6-membered monocyclic or 10-membered bicyclic aromatic ring, containing, where possible, 1, 2, 3 or 4 ring members independently selected from N, N(R12), S and O, optionally substituted as for heteroaryl^{a}.

74. A compound according to numbered embodiment 73, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
B is phenyl substituted as for aryl; or B is a 6-membered monocyclic or 10-membered bicyclic aromatic ring, containing 1 or 2 ring members independently selected from N or N(R12), optionally substituted as for heteroaryl^{a}.

75. A compound according to numbered embodiment 74, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
B is phenyl substituted with CN and optionally 1 or 2 further substituents as for aryl; or B is a 6-membered monocyclic or 9- or 10-membered bicyclic aromatic ring, containing, where possible, 1, 2, 3 or 4 ring members independently selected from N, N(R12), S and O, optionally substituted as for heteroaryl^{a}.

76. A compound according to numbered embodiment 75, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
B is phenyl substituted with CN and optionally 1 or 2 further substituents as for aryl; or B is a 6-membered monocyclic or 10-membered bicyclic aromatic ring, containing, where possible, 1, 2, 3 or 4 ring members independently selected from N, N(R12), S and O, optionally substituted as for heteroaryl^{a}.

77. A compound according to numbered embodiment 76, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
B is phenyl substituted with CN and optionally 1 or 2 further substituents as for aryl; or B is a 6-membered monocyclic or 10-membered bicyclic aromatic ring, containing 1 or 2 ring members independently selected from N or N(R12), optionally substituted as for heteroaryl^{a}.

78. A compound according to numbered embodiment 74 or a tautomer, isomer, stereoisomer (including an enantiomer, a diastereoisomer and a racemic and scalemic mixture thereof), a deuterated analogue, and a pharmaceutically acceptable salt and/or solvate thereof, wherein
B is phenyl optionally substituted as for aryl, or B is pyridyl or isoquinolinyl, optionally substituted as for heteroaryl^{a}.

79. A compound according to numbered embodiment 78 or a tautomer, isomer, stereoisomer (including an enantiomer, a diastereoisomer and a racemic and scalemic mixture thereof), a deuterated analogue, and a pharmaceutically acceptable salt and/or solvate thereof,
wherein B is phenyl substituted with CN and optionally 1 or 2 further substituents as for aryl, or B is pyridyl or isoquinolinyl, optionally substituted as for heteroaryl^{a}.

80. A compound according to numbered embodiment 78 or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is

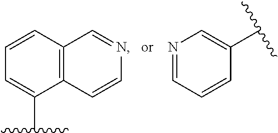 [SMILES: *c1cccc2cnccc12 ; *c1cccnc1]

or optionally substituted as for heteroaryl^{a}, or B is phenyl optionally substituted as for aryl.

81. A compound according to numbered embodiment 80 or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is

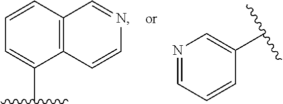 [SMILES: *c1cccc2cnccc12 ; *c1cccnc1]

or optionally substituted as for heteroaryl^{a}, or B is phenyl substituted with CN and optionally 1 or 2 further substituents as for aryl.

82. A compound according to any of the preceding numbered embodiments or a tautomer, isomer, stereoisomer (including an enantiomer, a diastereoisomer and a racemic and scalemic mixture thereof), a deuterated analogue, and a pharmaceutically acceptable salt and/or solvate thereof, wherein
B is unsubstituted; or wherein B is aryl, B is substituted with CN and optionally 1 or 2 further substituents as for aryl; or wherein B is heteroaryl^{a}, B is substituted with alkyl, halo, OH, or NH₂, and optionally 1 or 2 further substituents as for heteroaryl^{a}.

83. A compound according to numbered embodiment 78, or a tautomer, isomer, stereoisomer (including an enantiomer, a diastereoisomer and a racemic and scalemic mixture thereof), a deuterated analogue, and a pharmaceutically acceptable salt and/or solvate thereof, wherein
B is selected from

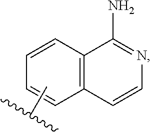

optionally further substituted with 1 or 2 substituents as for heteroaryl^{a};

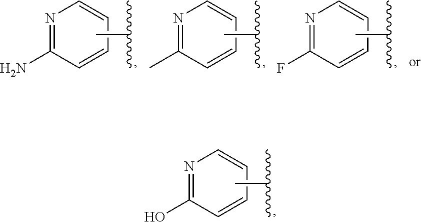

each optionally further substituted with 1 or 2 substituents as for heteroaryl^{a};

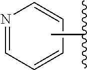

with no substituents; or

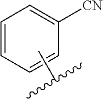

with no further substituents.

84. A compound according to numbered embodiment 83, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is selected from the following groups:

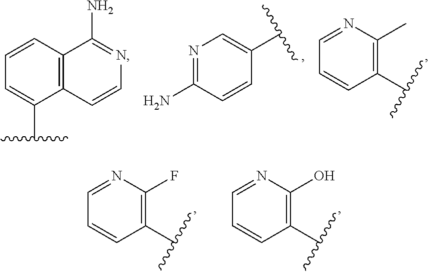

optionally further substituted with 1 or 2 substituents as for heteroaryl^{a}; or B is

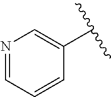 [SMILES: *c1cccnc1]

with no substituents; or B is

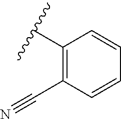 [SMILES: *c1ccccc1C#N]

with no further substituents.

85. A compound according to numbered embodiment 84, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is selected from

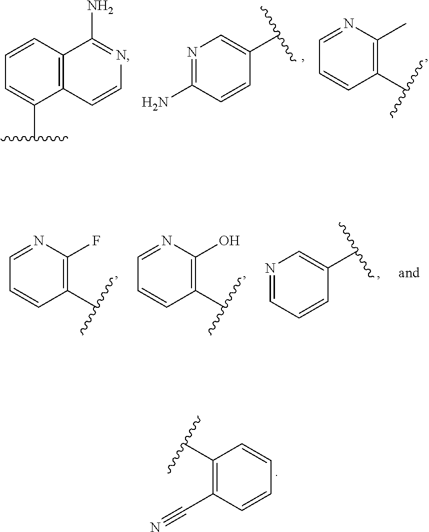

86. A compound according to any of numbered embodiments 1-68, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is heteroaryl^{a}.

87. A compound according to numbered embodiment 86 or a tautomer, isomer, stereoisomer (including an enantiomer, a diastereoisomer and a racemic and scalemic mixture thereof), a deuterated analogue, and a pharmaceutically acceptable salt and/or solvate thereof, wherein Y is attached to B at a carbon atom on the heteroaryl^{a} ring.

88. A compound according to numbered embodiment 86 or a tautomer, isomer, stereoisomer (including an enantiomer, a diastereoisomer and a racemic and scalemic mixture thereof), a deuterated analogue, and a pharmaceutically acceptable salt and/or solvate thereof, wherein Y is attached to B at a carbon atom on the heteroaryl^{a} ring, and the two ring atoms adjacent to the carbon atom on the heteroaryl^{a} ring to which Y attaches are both carbon.

89. A compound according to any of numbered embodiments 86 to 88, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is a 9 or 10-membered bicyclic aromatic ring, containing, where possible, 1, 2, 3 or 4 ring members independently selected from N, N(R12), S and O, optionally substituted as for heteroaryl^{a}.

90. A compound according to numbered embodiment 89, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is a 10-membered bicyclic aromatic ring, containing, where possible, 1, 2, 3 or 4 ring members independently selected from N, N(R12), S and O, optionally substituted as for heteroaryl^{a}.

91. A compound according to numbered embodiment 90, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is a 10-membered bicyclic aromatic ring, containing 1 or 2 ring members independently selected from N or N(R12), optionally substituted as for heteroaryl^{a}.

92. A compound according to numbered embodiment 91 or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is isoquinolinyl, optionally substituted as for heteroaryl^{a}.

93. A compound according to numbered embodiment 92 or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is

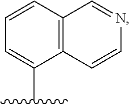 [SMILES: *c1cccc2cnccc12]

optionally substituted as for heteroaryl^{a}.

94. A compound according to any of numbered embodiments 86 to 93, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is substituted with NH₂, and optionally substituted with 1 or 2 further substituents as for heteroaryl^{a}.

95. A compound according to numbered embodiment 94, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is isoquinolinyl, substituted with NH₂ at the 1-position

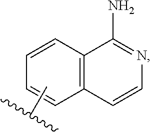

optionally further substituted with 1 or 2 substituents as for heteroaryl^{a}.

96. A compound according to numbered embodiment 95, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B is

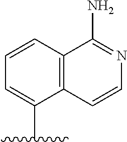 [SMILES: *c1cccc2c(N)nccc12]

optionally further substituted with 1 or 2 substituents as for heteroaryl^{a}.

97. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein alkoxy is a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from OH, CN, CF₃, and fluoro.

98. A compound selected from Table 1, Table 2, Table 3, Table 4, and Table 5, or a pharmaceutically acceptable salt, solvate, or solvate of a salt thereof.

99. A compound selected from Table 1; Table 2; compounds 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, and 92 of Table 3; compounds 93, 94, 95, 96, 97, 98, 99, and 101 of Table 4; and Table 5, or a pharmaceutically acceptable salt, solvate, or solvate of a salt thereof.

100. A pharmaceutical composition comprising: a compound, or a pharmaceutically acceptable salt and/or solvate thereof, according to any of numbered embodiments 1 to 99, and at least one pharmaceutically acceptable excipient.

101. A compound, or a pharmaceutically acceptable salt and/or solvate thereof, as defined in any of numbered embodiments 1 to 99, or the pharmaceutical composition according to numbered embodiment 100, for use in medicine.

102. A compound, or a pharmaceutically acceptable salt and/or solvate thereof, as defined in any of numbered embodiments 1 to 99, or the pharmaceutical composition according to numbered embodiment 100, for use in a method of treatment of a disease or condition in which Factor XIIa activity is implicated.

103. A compound, a pharmaceutically acceptable salt and/or solvate thereof, or a pharmaceutical composition for use as defined in numbered embodiment 102, wherein the disease or condition in which Factor XIIa activity is implicated is a bradykinin-mediated angioedema, wherein the bradykinin-mediated angioedema is hereditary angioedema.

104. A compound, a pharmaceutically acceptable salt and/or solvate thereof, or a pharmaceutical composition for use as defined in numbered embodiment 102, wherein the disease or condition in which Factor XIIa activity is implicated is a bradykinin-mediated angioedema, wherein the bradykinin-mediated angioedema is non hereditary.

105. A compound, a pharmaceutically acceptable salt and/or solvate thereof, or a pharmaceutical composition for use as defined in numbered embodiment 102, wherein the disease or condition in which Factor XIIa activity is implicated is selected from vascular hyperpermeability, stroke including ischemic stroke and haemorrhagic accidents; retinal edema; diabetic retinopathy; impaired visual acuity; DME; retinal vein occlusion; and AMD.

106. A compound, a pharmaceutically acceptable salt and/or solvate thereof, or a pharmaceutical composition for use as defined in numbered embodiment 102 wherein the disease or condition in which Factor XIIa activity is implicated is a thrombotic disorder.

107. A compound, a pharmaceutically acceptable salt and/or solvate thereof, or a pharmaceutical composition for use as defined in numbered embodiment 106, wherein the thrombotic disorder is thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; acute respiratory distress syndrome (ARDS); idiopathic pulmonary fibrosis (IPF); rheumatoid arthritis (RA); and cold-induced urticarial autoinflammatory syndrome.

108. A compound, a pharmaceutically acceptable salt and/or solvate thereof, or a pharmaceutical composition for use as defined in numbered embodiment 102 wherein the disease or condition in which Factor XIIa activity is implicated is neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

109. A compound, a pharmaceutically acceptable salt and/or solvate thereof, or a pharmaceutical composition for use as defined in numbered embodiment 102, wherein the disease or condition in which Factor XIIa activity is implicated is obesity or diabetes.

110. A compound, a pharmaceutically acceptable salt and/or solvate thereof, or a pharmaceutical composition for use as defined in numbered embodiment 102, wherein the disease or condition in which Factor XIIa activity is implicated is kidney disease, renal fibrosis, glomerulosclerosis, renal scarring, ischemia/reperfusion injury in native or transplant kidneys, and acute kidney injury.

### Synthetic Methods

The compounds of the present invention can be prepared according to the procedures of the following schemes and examples, using appropriate materials, and are further exemplified by the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. Those skilled in the art will readily understand that known variations of the conditions, processes and order in which the synthetic steps are performed in the following preparative procedures can be used to prepare these compounds.

The compounds and intermediates of the invention may be isolated in the form of their pharmaceutically acceptable salts, such as those described previously herein above. The interconversion between free form and salt form would be readily known to those skilled in the art.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 4^{th} Edition, 2006, may be used. For example, a common amino protecting group suitable for use herein is tert-butoxy carbonyl (boc), which is readily removed by treatment with an acid such as trifluoroacetic acid or hydrogen chloride in an organic solvent such as dichloromethane. Alternatively, the amino protecting group may be a benzyloxycarbonyl (Cbz or Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvent. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively, a trichloroethyl ester protecting group is removed with zinc in acetic acid. A common hydroxy protecting group suitable for use herein is a methyl ether, deprotection conditions comprise refluxing in 48% aqueous HBr, or by stirring with borane tribromide in an organic solvent such as DCM. Alternatively, where a hydroxy group is protected as a benzyl ether, deprotection conditions comprise hydrogenation with a palladium catalyst under a hydrogen atmosphere. Palladium catalysed cross coupling reactions to form carbon-carbon, carbon-nitrogen and carbon-oxygen bonds typically require a palladium catalyst and ligand capable of forming an in situ catalytically active LₙPd⁽⁰⁾ species. More recently, precatalysts have been identified that contain the desired ligand and a labile group capable of eliminating to rapidly generate the active LₙPd⁽⁰⁾ catalyst species in the reaction mixture (see, for example http://dx.doi.org/10.1039/c2sc20903a, http://dx.doi.org/10.1002/ange.201207750, http://dx.doi.org/10.1021/jo500355k, http://dx.doi.org/10.1021/ol401208t, http://dx.doi.org/10.1021/acs.orglett.8b00325). The choice of catalyst and ligand or precatalyst can be determined by literature or catalyst screening, by the skilled person.

The graphic representations of racemic, ambiscalemic and scalemic or enantiomerically pure compounds used herein are taken from Maehr J. Chem. Ed. 62, 114-120 (1985): solid wedges ( [symbol] ) and broken wedges ( [symbol] ) are used to denote the absolute configuration of a chiral element; solid bold lines ( [symbol] ) and broken bold lines ( [symbol] ) are geometric descriptors indicating the relative configuration shown, but denoting racemic character;

As used herein, a depiction including wedges or broken lines

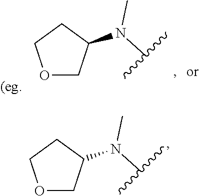

indicates that the structure encompasses purity of that relative or absolute configuration of at least 80% ee, preferably >90% ee.

As used herein, when a compound possesses a centre of asymmetry, its depiction with simple lines

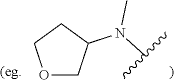 [SMILES: *N(C)C1CCOC1]

indicates that the structure includes any and all stereoisomers, without regard to enantiomeric purity.

The invention is illustrated by the following non-limiting examples in which the following abbreviations and definitions are used:

| | |
|---|---|
| AcOH | acetic acid |
| aq | aqueous solution |
| AIBN | azobisisobutyronitrile |
| boc | tert-butoxy carbonyl |
| Boc₂O | di-tert-butyl decarbonate |
| B₂Pin₂ | bis(pinacolato)diboron |
| BrettPhos | 2-(dicyclohexylphosphino)-3,6-dimethoxy-2′,4′,6′-triisopropyl-1,1′- |
| | biphenyl |
| BrettPhos Pd G3 | [(2-di-cyclohexylphosphino-3,6-dimethoxy-2′,4′,6′- triisopropyl-1,1′- |
| | biphenyl)-2-(2′-amino-1,1′ -biphenyl)]palladium(II) methanesulfonate |
| BrettPhos Pd G4 | methanesulfonato(2-dicyclohexylphosphino-3,6-dimethoxy-2′,4′,6′-tri-i- |
| | propyl-1,1′-biphenyl)(2′-methylamino-1,1′-biphenyl-2-yl)palladium(II) |
| | (CAS no. 1599466-83-7) |
| tBu | tert-butyl |
| tert-BuBrettphos Pd G3 | [(2-di-tert-butylphosphino-3,6-dimethoxy-2′,4′,6′-triisopropyl-1,1′- |
| | biphenyl)-2-(2′-amino-1,1′-biphenyl)]palladium(II) methanesulfonate |
| nBuLi | n-butyllithium |
| Cbz | benzyl carbamate |
| CDI | 1,1′-carbonyldiimidazole |
| Celite ® | Filter agent (diatomaceous earth) |
| COMU | 1-cyano-2-ethoxy-2-oxoethylidenaminooxy)dimethylamino- |
| | morpholino-carbenium hexafluorophosphate |
| DBU | 1,8-diazabicyclo[5.4.0]undec-7-ene |
| DCE | 1,2-dichloroethane |
| DCM | dichloromethane |
| DEA | diethylamine |
| Dess-Martin | 1,1,1-tris(acetyloxy)-1,1-dihydro-1,2-benziodoxol-3-(1H)-one |
| periodinane | |
| DIAD | diisopropyl azodicarboxylate |
| DIPEA | N,N-diisopropylethylamine |
| DMF | N,N-dimethylformamide |
| DMSO | dimethyl sulfoxide |
| EDC | 1-ethyl-3-(3-dimethylaminopropyl)carbodiimide hydrochloride |
| eq | equivalent |
| Et₂O | diethyl ether |
| Et | ethyl |
| EtOH | ethanol |
| EtOAc | ethyl acetate |
| HATU | 2-(3H-[1,2,3]triazolo[4,5-b]pyridin-3-yl)-1,1,3,3-tetramethylisouronium |
| | hexafluorophosphate(V) |
| h | hours |
| HOBt | 1-hydroxybenzotriazole hydrate |
| IPA | isopropyl alcohol |
| LCMS | liquid chromatography mass spectrometry |
| LIHMDS | lithium hexamethyldisilazide |
| Me | methyl |
| MeCN | acetonitrile |
| Mel | iodomethane |
| MeOH | methanol |
| min | minutes |
| MS | mass spectrum |
| Ms | methanesulfonyl |
| MsCl | methanesulfonyl chloride |
| NBS | N-bromosuccinimide |
| NCS | N-chlorosuccinimide |
| NMR | nuclear magnetic resonance spectrum |
| NMP | N-methyl-2-pyrrolidone |
| OAc | acetate |
| Pd-175 | [′BuBrettPhos Pd(allyl)]OTf, allyl(2-di-tert-butylphosphino-3,6- |
| | dimethoxy-2′,4′,6′-triisopropyl-1,1′-biphenyl)palladium(II) triflate |
| Pd(dppf)Cl₂:DCM | [1,1′-bis(diphenylphosphino)ferrocene]dichloropalladium(II), complex |
| | with dichloromethane |
| Pd-PEPPSI-IPent | [1,3-bis(2,6-di-3-pentylphenyl)imidazol-2-ylidene](3- |
| | chloropyridyl)palladium(II) dichloride |
| Pet. Ether | petroleum ether fraction boiling at 60-80 °C |
| Ph | phenyl |
| Pin | pinacol |
| PyBOP | benzotriazole-1-yl-oxy-tris-pyrrolidino-phosphonium |
| | hexafluorophosphate |
| iPr | iso-propyl |
| nPr | n-propyl |
| RockPhos Pd G3 | [(2-di-tert-butylphosphino-3-methoxy-6-methyl-2′,4′,6′-triisopropyl- |
| | 1,1′-biphenyl)-2-(2-aminobiphenyl)]palladium(II) methanesulfonate |
| RuPhos Pd G3 | (2-dicyclohexylphosphino-2′,6′-diisopropoxy-1,1′-biphenyl) [2-(2′-amino- |
| | 1,1′-biphenyl)]palladium(II) methanesulfonate |
| RuPhos | 2-dicyclohexylphosphino-2′,6′-diisopropoxybiphenyl |
| sat. | saturated |
| SCX | strong cation exchange cartridge |
| SEM-Cl | 2-(trimethylsilyl)ethoxymethyl chloride |
| SFC | supercritical fluid chromatography |
| STAB | sodium triacetoxyborohydride |
| SWFI | sterile water for injection |
| rt | room temperature |
| TBAB | tetra-n-butylammonium bromide |
| TBAF | tetra-n-butylammonium fluoride |
| TBDMS | tert-butyldimethylsilyl |
| TBME | tert-butyl methyl ether |
| TBDPS | tert-butyldiphenylsilyl |
| THF | tetrahydrofuran |
| TEA | triethylamine |
| TFA | trifluoroacetic acid |
| Z | benzyl carbamate |

All reactions were carried out under an atmosphere of nitrogen unless specified otherwise.

Hydrogenations were typically carried out using an H-Cube® reactor (manufactured by Thalesnano, Inc, Hungary).

References to the use of microwave, a microwave reactor, microwave heating and microwave irradiation all refer to the use of a CEM Discover Microwave Reactor.

References to the use of a phase separator refer to columns fitted with a selectively permeable, optimized frit material that separates aqueous phase from an organic phase under gravity.

¹H NMR spectra were recorded using instrumentation selected from
Bruker (500 MHz or 400 MHz) spectrometer with a Bruker Avance II or Avance 11 console
Oxford (400 MHz) AS400 magnet with a Inova console
300 MHz Varian Mercury+ NMR System
   and reported as chemical shift (ppm). It will be understood that, where exchangeable protons are present in any compound, the number of protons in the ¹H NMR spectra may not exactly correspond to the number of protons in the structure of any compound synthesised herein.

Molecular ions were obtained using LCMS with appropriate conditions selected from
Chromolith Speedrod RP-18e column, 50×4.6 mm, with a linear gradient 10% to 90% 0.1% HCO₂H/MeCN into 0.1% HCO₂H/H₂O over 13 min, flow rate 1.5 mL/min;
Agilent, X-Select, acidic, 5-95% MeCN/water over 4 min. Data was collected using a Thermofinnigan Surveyor MSQ mass spectrometer with electrospray ionisation in conjunction with a Thermofinnigan Surveyor LC system;
LCMS (Waters Acquity UPLC, C18, Waters X-Bridge UPLC C18, 1.7 μm. 2.1×30 mm, Basic (0.1% Ammonium Bicarbonate) 3 min method;
LCMS (Agilent, X-Select, Waters X-Select C18, 2.5 μm, 4.6×30 mm, Acidic 4 min method, 95-5 MeCN/water);
LCMS (Agilent, Basic, Waters X-Bridge C18, 2.5 μm, 4.6×30 mm, Basic 4 min method, 5-95 MeCN/water;
Acquity UPLC BEH C18 1.7 μM column, 50×2.1 mm, with a linear gradient 10% to 90% 0.1% HCO2H/MeCN into 0.1% HCO2H/H2O over 3 min, flow rate 1 mL/min. Data was collected using a Waters Acquity UPLC mass spectrometer with quadropole dalton, photodiode array and electrospray ionisation detectors;
Agilent 1100 LC/MSD with Kinetex® 5 μm EVO C18 100 Å LC 50×4.6 mm and Gemini® 5 μm NX-C18 110 Å LC 50×4.6 mm columns. Acidic mobile phases used a linear gradient of 5-95% 10 mM aq. NH₄HCO₂/MeCN and basic mobile phases used a linear gradient of 5-95% 10 mM aq. NH₄HCO₃/MeCN. Samples were run over 3 minutes, using a flow rate of 2.2 mL/min, and a pressure range of 0-200 bar. Data was collected using a Waters 3100 Mass Detector with single high resolution quadrupole and photomultiplier detectors and High Performance Zspray™ dual-orthoganal API sources for standard ESI, or multimode ESI/APCI/ESCi®.
UPLC (CSH C18 Column, 130 Å, 1.7 μm, 2.1 mm×30 mm, 3 min method, 0.1% Formic acid, 2-100% MeCN/water)
LCMS (Cortecs C18+, 90 Å, 2.7 μm, 2.1 mm×30 mm, 3 min method, 0.1% Formic acid, 5-100% MeCN/water)
UPLC (BEH C18 Column, 130 Å, 1.7 μm, 2.1 mm×30 mm, 3 min method, 0.1% Ammonium Hydroxide, 2-100% MeCN/water)
LCMS (Kinetex Evo C18, 130 Å, 2.5 μm, 2.1 mm×30 mm, 3 min method, 0.1% Ammonium Hydroxide, 5-100% MeCN/water)

Flash chromatography was typically carried out over ‘silica’ (silica gel for chromatography, 0.035 to 0.070 mm (220 to 440 mesh) (e.g. Merck silica gel 60)), and an applied pressure of nitrogen up to 10 p.s.i accelerated column elution. Alternatively, pre-prepared cartridges of silica gel were used, for example pre-packed SiliaSep™ columns from Silicycle or Sfär C18 D—Duo 100 Å 30 μm columns from Biotage. Typical conditions included, for example, flow rate range of 18-200 mL/min, with an applied pressure range of 0-225 PSI. Hexanes, EtOAc, DCM, MeOH, were used as mobile phases for normal-phase chromatography purifications. MeOH, MeCN, 10 mM Ammonium Formate pH 4 in H₂O and 10 mM Ammonium Bicarbonate pH 10 in H₂O buffers were used as mobile phases for reverse-phase chromatography. It will be understood that alternative conditions (such as flow rate ranges, applied pressures, solvents and pH) may be used for flash chromatography in order to separate and purify compounds synthesised herein.

The term “prep HPLC” refers to reverse phase preparative HPLC purifications. Typical instrumentation and conditions include, for example, Waters Fraction Lynx Prep-HPLC with MWD/DAD & MSD using
5-95% acetonitrile/methanol with 0.1% NH₄OH pH 10 in H₂O as a basic mobile phase
5-95% acetonitrile/methanol with 0.1% HCO₂H pH 3 in H₂O as an acidic mobile phase

It will be understood that alternative conditions (such as choice of column, flow rate ranges, solvents, modifiers and pH) may be used for prep HPLC in order to separate and purify compounds synthesised herein.

The procedure of lyophilisation (or freeze drying) is generally well known in the art. Typically the substance is taken up in water, if necessary, with the addition of a minimum amount of MeCN to aid dissolution, and frozen, typically by rapid cooling in a cold bath at −78 °C. The resulting frozen solid mixture is evaporated to dryness in vacuo.

The term “concentrated” refers to evaporation of solvent under reduced pressure using a rotary evaporator, heating where necessary.

All solvents and commercial reagents were used as received.

IUPAC chemical names were generated using automated software such as Dotmatics Studies Notebook or ChemDraw (PerkinElmer). Compounds produced by the methods below may be isolated in salt forms. However, compound naming used herein typically refers to the compound without any salt counter ion.

The example compounds described herein can be prepared using conventional synthetic methods for example, but not limited to, the routes outlined in the General Schemes below, using, for example, the General Methods below.

### General Methods

### 1. General Method 1: (GM1): Mesylation and Chlorination

a. General Method 1a (GM1a): Mesylation

A solution of alcohol (1.0 eq) in DCM was cooled in an ice/water bath and MsCl (1.2 eq) was added dropwise followed by TEA (1.4 eq) maintaining cooling. The reaction was stirred at rt for 2-18 h. The reaction was diluted with DCM and washed with water. The aqueous layer was extracted with DCM (×3) and the combined organics were washed with brine, dried (Na₂SO₄), filtered and concentrated. The crude product was used without purification or purified by flash chromatography.

b. General Method 1b (GM1b): Chlorination Via a Mesylate

MsCl (2.5 eq) was added to a solution of TEA (2.8 eq) and alcohol (1.0 eq) in DCM while cooling in an ice/water bath. The reaction was stirred at rt for 18 h. The reaction was diluted with DCM and washed with sat. NaHCO₃ (aq). The aqueous layer was extracted with DCM (×3) and the combined organics were washed with brine, dried (Na₂SO₄), filtered and concentrated. The crude product was purified by flash chromatography.

c. General Method 1c (GM1c): Chlorination Via NCS

A solution of indole or azaindole (1.0 eq) in DCM was protected from light and treated with NCS (3.75 eq) at rt for 12-48 h. The mixture was treated with 1M HCl (aq) and the phases separated. The organic phase was washed with brine, dried (Na₂SO₄), filtered, concentrated and purified by flash chromatography.

### 2. General Method 2 (GM2): SN₂ Alkylation (0 and N)

a. General Method 2a (GM2a): SN₂ Alkylation: NaH

To a suspension of NaH (60% wt. on mineral oil) (1.1 eq) in DMF in an ice/water bath was added a solution of alcohol, pyrrole or indole (1.0 eq) in DMF dropwise over 2 min. The mixture was allowed to warm to rt for 5 min before cooling again in an ice/water bath and treating with a solution of the alkylhalide or mesylate (1.0 eq) in DMF over 2 min. The mixture was maintained in an ice/water bath for 1 h before being allowed to warm to rt, or heated at elevated temperature and stirred for 2-18 h. Sat. NH₄Cl (aq) or sat. NaHCO₃ (aq) was added and extracted with EtOAc (×3). The organic phases were combined, dried (MgSO₄), filtered and concentrated. The crude product was purified by flash chromatography.

b. General Method 2b (GM2b): SN₂ Alkylation; Cs₂CO₃ or K₂CO₃

A solution of alkyl halide or mesylate (1-2 eq), amine (1.0 eq), and base such as K₂CO₃, or Cs₂CO₃ (2.5 eq), in a solvent such as DMF, DMSO or MeCN, was stirred at 80 °C for 2-18 h. MecOH (5 mL) was added, and the mixture was diluted with water. The product was extracted into EtOAc (×2) and washed with brine.

The organic layer was dried (Na₂SO₄), filtered and concentrated. The product was either used directly or purified by flash chromatography.

### 3. General Method 3 (GM3): Reduction

a. General Method 3a (GM3a): Nitrile Reduction; H-Cube® with Pd/C or Raney Ni Cartridge

The nitrile was dissolved in a 0.5M NH₃/MeOH solution and passed through an H-Cube® reactor (Pd/C or Raney Ni cartridge) at 50 °C, ‘full’ hydrogen delivery mode (50 bar), flow rate: 1 mL/min. The reaction was concentrated to afford the product which was used without further purification.

b. General Method 3b (GM3b): Nitrile, Amide and Ester Reduction; LiAIH₄ in THF

To a solution of amide, nitrile, or ester (1.0 eq) in THF in an ice/water bath was added LiAIH₄ (2M in THF) (2.0 eq) dropwise and the reaction mixture was allowed to warm to rt then stirred for 4-18 h. The reaction mixture was cooled in an ice/water bath, treated portionwise with Na₂SO₄·10H₂O (3.5 eq) and stirred for 30 min before being dried (MgSO₄), filtering and washing with THF. The filtrate was concentrated to afford the crude product which was used without purification or purified by flash chromatography.

c. General Method 3c: borane-THF reduction

A solution of nitrile (1.0 eq) in THF was cooled in an ice/water bath before borane (1M in THF, 2.0 eq) was added dropwise. The reaction was allowed to warm to rt then heated to 60 °C for 16-96 h. MeOH was added and heating continued at 60 °C for 24 h before cooling to rt and concentrating. The product was isolated and purified using one of the following methods:
i) The crude product was loaded onto an SCX in MeOH and washed with MeOH. The product was eluted with 7M NH₃ in MeOH and the eluent concentrated.
ii) The crude product was purified by flash chromatography
iii) Boc₂O (1.2 eq) was added to the crude reaction mixture and stirred overnight. The solvent was concentrated. The product was taken up in DCM, washed with water and brine, dried (Na₂SO₄), filtered and concentrated. The boc-protected amine was either used without further purification or purified by flash chromatography
   d. General Method 3d: NiCl₂ Reduction

A solution of nitrile (1.0 eq), NiCl₂·6H₂O (1.0 eq) and Boc₂O (3.0 eq) in MeOH was cooled in an ice/water bath and sodium borohydride (NaBH₄) (5.0 eq) added portionwise. The reaction was allowed to warm to rt and stirred for 18 h. Water was added, and the reaction mixture filtered, washed with THF and concentrated. The crude product was purified by flash chromatography.

e. General Method 3e: Hydrogenation; Pd/C

To a solution of nitrile (1.0 eq) in MeOH or EtOH under an inert atmosphere was added 10% Pd/C (0.1-0.2 eq). Additives such as HCl, sulfuric acid, or Boc₂O may optionally be added. The reaction was stirred under an atmosphere of H₂ (g) for 2-72 h. The catalyst was removed by filtration over Celite®, which was washed with EtOH. The product was isolated following concentration of the filtrate and used directly or purified by flash chromatography.

f. General Method 3f: Aromatic Ring Reduction

A biaryl ring (1.0 eq) was dissolved in EtOH and subjected to hydrogenation in the H-Cube® at 70 °C, 50 bar, 1 mL/min using a 10% Pd/C CatCart, recirculating when necessary. The solvent was removed in vacuo to afford the product which was used without purification.

### 4. General Method 4 (GM4): Buchwald Coupling

A suspension of amine or alcohol (1.0 eq), aryl halide (1.1 eq) and a base such as Cs₂CO₃, NaOtBu (2.0 eq) or LiHMDS (2.0 eq) in a degassed solvent such as THF or 1,4-dioxane was purged with N₂ (g). A Buchwald palladium precatalyst, such as BrettPhos Pd G3, (0.11 eq) was added and the mixture degassed and purged with N₂ (g) for 5 min. The reaction was heated in a sealed vial at rt—60 °C for 30 min-3 days as required. The product was isolated and purified using one of the following methods:
i) The reaction was quenched with AcOH (2.0 eq) and concentrated. The crude was purified by SCX, loading and washing with MeOH then eluting the product with NH₃ in MeOH followed by purification by flash chromatography or prep HPLC.
ii) The reaction was quenched with AcOH (2.0 eq), filtered through Celite®, washing with EtOAc and the filtrate concentrated. The crude product was purified by flash chromatography.
iii) The reaction mixture was acidified with AcOH (2.0 eq) and stirred for 5 min, 1M NH₃ in MeOH was added and the reaction mixture was concentrated on to silica and purified by flash chromatography.
iv) The reaction mixture was dry loaded on to silica and purified by flash chromatography.
v) The reaction was concentrated, and the product taken forward to deprotection without purification.

### 5. General Method 5 (GM5): Boc Deprotection; HCl or TFA

a. General Method 5a (GM5a): Boc Deprotection; HCl/Dioxane

A suspension of boc protected amine (1.0 eq) in 1,4-dioxane was treated with 4M HCl in dioxane (10.0 eq) and the reaction stirred at rt for 2-24 h. The product was isolated and purified using one of the following methods:
i) The reaction mixture was concentrated, optionally azeotroping with Et₂O or toluene to afford the product as a hydrochloride salt.
ii) The reaction mixture was concentrated, and the product converted to free base using a bicarbonate cartridge, PL-HCO₃ MP SPE (Agilent), loading in MeOH. The filtrate was concentrated and triturated with Et₂O to afford the product.
   b. General Method 5b (GM5b): Boc Deprotection; TFA

A mixture of boc protected amine (1.0 eq) in DCM was treated with TFA (10.0 eq) and stirred at rt for 2 h. The product was isolated and purified using one of the following methods:
i) The mixture was passed directly through an SCX column and washed with MeOH. The product was eluted with a solution of 7M NH₃ in MeOH and concentrated. The crude product was purified by flash chromatography or prep HPLC.
ii) The mixture was concentrated and loaded onto a PL-HCO3 MP SPE cartridge eluting with a mixture of MeCN and MeOH (1:1 v/v, 3×2 mL). The crude product was purified by flash chromatography or prep HPLC.
iii) The mixture was diluted with aq. NaOH (2M, 10 mL) and a mixture of DCM and MeOH (9:1 v/v, 20 mL). The phases were separated and the aq. Phase was extracted with a mixture of DCM and MeOH (9:1 v/v). The combined organic phases were dried and concentrated. The residue was purified by flash chromatography or prep HPLC
iv) The mixture was diluted with aq. NaOH (2M, 10 mL) and a mixture of DCM and MeOH (9:1 v/v, 20 mL). The phases were separated, and the aqueous phase was extracted with a mixture of DCM and MeOH (9:1 v/v). The combined organic layers were dried (Na₂SO₄), filtered, and concentrated. Product was purified by PL-HCO₃ MP SPE cartridge eluting with a mixture of MeCN and MeOH (1:1 v/v), followed by flash chromatography or prep HPLC.

### 6. General Method 6 (GM6): Pyridone Chlorination

Pyridone (1.0 eq) was suspended in phosphorus oxychloride (large excess) and heated at reflux for 4 h. The reaction mixture was evaporated then azeotroped with toluene (×2). The residue was used immediately in the next step, taking care to exclude moisture.

### 7. General Method 7 (GM7): SNAr Alkylation (0 and N)

a. General Method 7a (GM7a): SNAr O-Alkylation Using NaH

To a suspension of NaH (60% wt. on mineral oil) (1.04 eq) in DMF in an ice/water bath was added a solution of alcohol (1.02 eq) in DMF dropwise over 2 min. The mixture was allowed to warm to rt for 5 min before cooling again in an ice/water bath and treating with pyridyl halide or aryl halide (1.0 eq). The reaction mixture was maintained in an ice/water bath for 1 h then warmed to rt for 18 h. The reaction mixture was cooled in an ice/water bath and sat. Na₂CO₃ (aq) was added followed by water. This was extracted with EtOAc (×3) and the organic phases were combined, washed with 1:1 water/brine and brine. The organic phase was dried (MgSO4), filtered and concentrated. The crude product was purified by flash chromatography.

b. General Method 7b (GM7b): SNAr O-Alkylation Using Cs₂CO₃

To a solution of alcohol (1.0 eq) and pyridyl halide (1.0 eq) in MeCN was added Cs₂CO₃ (2.0 eq) and the mixture was stirred in a sealed vial at 50 °C for 18-72 h. The product was isolated and purified using one of the following methods
i) The reaction mixture was cooled to rt and diluted with water (10 mL). The crude product was extracted into DCM, dried (MgSO₄), filtered and concentrated. The residue was purified by flash chromatography
ii) The reaction mixture was filtered through Celite® and the filtrate was concentrated to yield the crude product which was either used without further purification or purified by flash chromatography
iii) The crude reaction mixture was passed directly through an SCX column, loading in MeOH. The SCX was washed with MeOH and the product was eluted with 7M NH₃ in MeOH. The crude product was purified by flash chromatography.
   c. General Method 7c (GM7c): SNAr O-Alkylation Using NaO^{t}Bu

A solution of alcohol (1.0 eq), aryl halide (1.0 eq) and NaO^{t}Bu (3.0 eq) in NMP was stirred in the microwave at 140 °C for 4 h. The crude reaction mixture was loaded onto an SCX column in MeOH and washed with MeOH and the product was eluted with 7M NH₃ in MeOH (50 mL). The product was concentrated and purified by flash chromatography or prep HPLC.

d. General Method 7d (GM7d): SNAr N-Alkylation

Amine (1.0 eq) and halopyridine (1.0 eq) were dissolved in MeCN. K₂CO₃ (3.0 eq) was added and the reaction was stirred at 60-120 °C for 60-90 min under thermal heating or microwave irradiation. The reaction was diluted with water and extracted with IPA/CHCl₃ (1:10) (×3). The combined organics were washed with brine, dried (MgSO₄) and concentrated. The product was isolated and used directly or purified by flash chromatography.

### 8. General Method 8 (GM8): 2,4-Dimethoxybenzyl Deprotection

A solution of 2,4-dimethyoxybenzyl protected amine (1.0 eq) in TFA (10 eq.) was stirred at rt—50 °C for 60 min.

The product was isolated and purified using one of the following methods:
i) The reaction mixture was concentrated. The resulting residue was suspended in MeOH (2 mL) and loaded onto SCX, which was flushed with MeOH. The product was eluted with a solution of 7M NH₃ in MeOH. The solvent was concentrated and the product was purified by flash chromatography or prep HPLC.
ii) The mixture was concentrated and loaded onto a PL-HCO3 MP SPE cartridge eluting with a mixture of MeCN and MeOH (1:1 v/v, 3×2 mL). The crude product was purified by flash chromatography or prep HPLC.
iii) The mixture was diluted with NaOH (aq) (2M, 10 mL) and a mixture of DCM and MeOH (9:1 v/v, 20 mL). The phases were separated, and the aqueous phase was extracted with a mixture of DCM and MeOH (9:1 v/v). The combined organic phases were dried and concentrated. The residue was purified by flash chromatography or prep HPLC
iv) The mixture was diluted with NaOH (aq) (2M, 10 mL) and a mixture of DCM and MeOH (9:1 v/v, 20 mL). The phases were separated, and the aqueous phase was extracted with a mixture of DCM and MeOH (9:1 v/v). The combined organic layers were dried (Na₂SO₄), filtered, and concentrated. Product was purified by PL-HCO₃ MP SPE cartridge eluting with a mixture of MeCN and MeOH (1:1 v/v), followed by flash chromatography or prep HPLC.

### 9. General Method 9 (GM9): Carbamate Protection

To a solution of aminopyridine (1.0 eq) and TEA (2.0 eq) in DCM in an ice/water bath was added methylchloroformate (3.0 eq) and the reaction was stirred at rt for 48 h. The reaction mixture was diluted with DCM and washed with water. The aqueous was extracted with DCM (×3) and the combined organics dried (Na₂SO₄), filtered and concentrated. The crude product was triturated with EtOAc.

### 10. General Method 10 (GM10): Carbamate Deprotection

a. General Method 10a: KOH

A mixture of methyl carbamate (1 eq) and KOH (6 eq) in MeOH was stirred at 60 °C for 12-48 h. The product was isolated and purified using one of the following methods:
i) The reaction was quenched with AcOH (6.0 eq) and the mixture was left to stir for 5 min before being concentrated. The residue was passed directly through an SCX in MeOH. The SCX was washed with MeOH and the product was eluted with 7M NH₃ in MeOH and lyophilised.
ii) The reaction was quenched with AcOH (6.0 eq) and the mixture was left to stir for 5 min before being concentrated. The residue was passed directly through an SCX in MeOH. The SCX was washed with MeOH and the product was eluted with 7M NH₃ in MeOH. The product was purified by flash chromatography or prep HPLC
iii) The reaction was quenched with AcOH (6.0 eq), concentrated, and purified by prep HPLC.
   b. General Method 10b: LiOH

To a solution of methyl carbamate (1 eq) in THF/water (10:1) was added lithium hydroxide monohydrate (3-5 eq) and the reaction stirred at 60 °C for 18 h-4 days. The mixture was cooled to rt and concentrated. The crude residue was purified via flash chromatography or prep HPLC

### 11. General Method 11: SEM Deprotection

TFA (10 eq) was added dropwise to a rapidly stirred solution of indole or azaindole (1.0 eq) in DCM. The mixture was stirred at rt for 18 h. The reaction mixture was passed directly through an SCX in MeOH. The SCX was washed with MeOH and the product was eluted with 2.5M NH₃ in MeOH and concentrated. The crude product was purified by flash chromatography or prep HPLC.

### 12. General Method 12: Alkylation and Cyclisation

To a solution of the amino substituted heteroaryl (e.g. pyridine) (1 eq) in EtOH (3 mL) were added NaHCO₃ (2 eq) and the chloromethyl aldehyde or chloromethyl ketone as applicable (2 eq). The mixture was stirred for 20 h at 75 °C. The mixture was then cooled to rt, filtered through Celite® washing with EtOAc (50 mL) and concentrated. The product was purified by flash chromatography.

### 13. General Method 13: Hydroxylation

A solution of the aryl bromide or heteroaryl bromide (1.0 eq), B₂Pin₂ (2.0 eq) and Pd(dppf)Cl₂ (0.1 eq) in anhydrous, degassed 1,4-dioxane was purged with N₂ (g) for 5 min. KOAc (3.0 eq) was added and the reaction stirred at 90 °C for 2-18 h. AcOH (2.0 eq) and water (1 mL) were added and the reaction stirred at rt for 15 min-18 h. A solution of H₂O₂ in water (30% w/w, 2 eq) was added and the reaction mixture stirred for 1-18 h.
i) Typically for basic compounds: solid Na₂S2O₃ was added and the reaction was stirred at rt for 5 min then diluted with EtOAc, filtered through Celite® and concentrated. The residue was purified by SCX and flash chromatography.
ii) Typically for non basic compounds: sat. Na₂S2O₃ (aq) and sat. NaHCO₃ (aq) were added and the aqueous was extracted with EtOAc. The combined organics were washed with brine, dried (MgSO₄), concentrated, and purified by flash chromatography

### 14. General Method 14: Esterification

To a stirred suspension of carboxylic acid (1.0 eq) in MecOH in an ice/water bath was added thionyl chloride (1.1 eq) dropwise. The reaction was warmed to rt and stirred for 20 h. The solvent was concentrated and trituration with Et₂O afforded the product.

### 15. General Method 15: Mitsunobu

To a mixture of alcohol (1.0 eq) and phenol (1.1 eq) in anhydrous THF at 0 °C was added PPh₃ (1.5 eq) followed by DIAD (1.5 eq). The mixture was stirred at 0 °C for 10 min then warmed to rt and stirred for 24 h and then concentrated. The residue was diluted with NaOH (aq) (1M) and DCM. The phases were separated, and the aqueous phase was extracted with DCM (×2). The combined organic phases were washed with sat. NaHCO₃ (aq) and brine, dried (Na₂SO₄), filtered and concentrated. Purification by flash column chromatography afforded the product. The crude product was either used without further purification or purified by flash chromatography or prep HPLC.

### 16. General Method 16: Oxidation

To a solution of alcohol (1.0 eq) in DCM at 0 °C was added Dess-Martin periodinane (2.0 eq). The mixture was stirred at rt for 18 h. A further portion of Dess-Martin periodinane (0.2 eq) was added to the solution at 0 °C. The mixture was stirred at rt for 4 h and diluted with sat. NaHCO₃ (aq) (50 mL) and sat. Na₂S2O₃ (aq) (50 mL) at rt. The mixture was stirred for 15 min at rt and the phases were separated. The aqueous phase was extracted with DCM (×2) and the combined organic phases were washed with sat. Na₂S2O₃ (aq), sat. NaHCO₃ (aq), brine, dried (Na₂SO₄), filtered and concentrated to afford the product.

### 17. General Method 17 (GM17): Reductive Amination

To a solution of aldehyde (1.0 eq) and amine (1.10 eq) in DCE was added AcOH (1.15 eq) and the mixture stirred for 30 min then STAB (2.0 eq) was added. The mixture was stirred for 18-72 h at rt before quenching with sat. NaHCO₃ (aq) and extracting with DCM (×3). The combined organic layers were dried (Na₂SO₄), filtered, and concentrated. Purification by flash chromatography afforded the product.

18. General Method 18 (GM18): Amide coupling
a. General Method 18a (GM18a): Amide Coupling Using HATU

To a solution of carboxylic acid (1.2 eq) in anhydrous DMF was added DIPEA (3 eq) and HATU (1.05 eq). The reaction was stirred at rt for 40 min then amine (1.0 eq) was added and stirring continued for 60 min. Concentration afforded the product which was either used without further purification or purified by flash chromatography or prep HPLC

b. General Method 18b (GM18b): Amide coupling using PyBop

To a mixture of carboxylic acid (1.0 eq) in DCM was added amine (1.0 eq), PyBOP (1.2 eq) and DIPEA (3.0 eq). The mixture was stirred at rt for 16 h and then concentrated. The residue was partitioned between sat. NaHCO₃ (aq) and EtOAc. The aqueous was washed with EtOAc (×3). The combined organic layers were dried (Na₂SO₄), filtered and concentrated. Purification by flash chromatography or prep HPLC afforded the product.

### 19. General Method 19 (GM19): Sulfonamide Formation

To a solution of amine (1.0 eq) and TEA (2.3 eq) in anhydrous DCM (2 mL), cooled in an ice/water bath, was added sulfonyl chloride (2.0 eq). The mixture was stirred in an ice/water bath for 2 h. Concentration afford the product. The crude product was either used without further purification or purified by flash chromatography or prep HPLC.

### 20. General Method 20 (GM20): Carbamate Formation

a. General Method 20a (GM20a): Carbamate Formation Using CDI

To a solution of CDI (1.5 eq) in anhydrous MeCN at rt was added alcohol (1.0 eq). The mixture was stirred for 2 h and then concentrated. The residue was partitioned between H₂O and DCM and the aqueous washed with DCM (×2). Combined organics were dried (Na₂SO₄), filtered and concentrated. The intermediate (2.0 eq) was dissolved in anhydrous THF and amine (1.0 eq) added followed by DBU (0.3 eq). The mixture was heated to 65 °C for 18 h-4 days then cooled and concentrated. The residue was partitioned between H₂O and DCM and the aqueous washed with DCM (×2). Combined organics were dired (Na₂SO₄), filtered and concentrated. Purification by flash chromatography or prep HPLC afforded the product.

b. General Method 20b (GM20b): Carbamate Formation Using Phosgene

To a stirred solution of alcohol (1.06 eq) and TEA (1.56 eq) in anhydrous DCM at rt was added phosgene (20% in toluene, 1.02 eq) and the reaction stirred for 1 h. A solution of amine (1.0 eq) and TEA (1.55 eq) in anhydrous DCM was added and stirring continued for 24 h. The mixture was quenched with 1 M NaOH and brine (1:1), and the layers were separated. The aqueous layer was extracted with DCM (×2), and the combined organic layers were dried (Na₂SO₄), filtered, and concentrated. The crude product was purified by flash chromatography.

### 21. General Method 21 (GM21): Urea Formation

a. General Method 21a (GM21a): Urea Formation Using CDI

To a solution of amine (1.6 eq) in DMF was added CDI (1.5 eq) and DIPEA (3.6 eq). The mixture was stirred at rt for 2 h then a solution of scaffold amine (1.0 eq) in DMF was added. The mixture was stirred at rt for 72 h and then concentrated. The crude product was either used without further purification or purified by flash chromatography or prep HPLC.

b. General Method 21b (GM21b): Urea Formation Using Triphosgene
Part 1: A mixture of amine (1.6 eq) in anhydrous THF was added to a mixture of triphosgene (0.65 eq) and pyridine (2.2 eq) in anhydrous THF at rt. The mixture was stirred for 18-72 h.
Part 2: To the reaction mixture was added a mixture of scaffold amine (1.0 eq) and TEA (3 eq) in anhydrous DMSO. The reaction was stirred for 1-36 h. The product was isolated and purified using one of the following methods:
   i) The reaction mixture was concentrated and purified by flash chromatography.
   ii) The reaction was diluted with brine and extracted with a mixture of DCM and MeOH (9:1 v/v). The combined organic phases were washed with brine, dried (Na₂SO₄), filtered and concentrated. The crude product was either used without further purification or purified by flash chromatography or prep HPLC
      c. General Method 21c (GM21c): Urea Formation Using Isocyanates

To a solution of amine (1.0 eq) and TEA (3.0 eq) in DMSO was added isocyanate (1.1 eq), and the mixture was stirred for 16 h. The mixture was quenched with MeOH, filtered and purified by automated prep HPLC.

### General Schemes

General Schemes 1-6 outline synthetic routes for certain example compounds and RgA, RgB, RgC and RgD refer to various substituents as required by the examples. For the sake of clarity General Schemes 1-6 are drawn with an oxabicyclohexyl central core group. Similar chemistry can be applied to examples with different core groups such as bicyclopentyl, azabicyclohexyl, and oxabicycloheptyl.

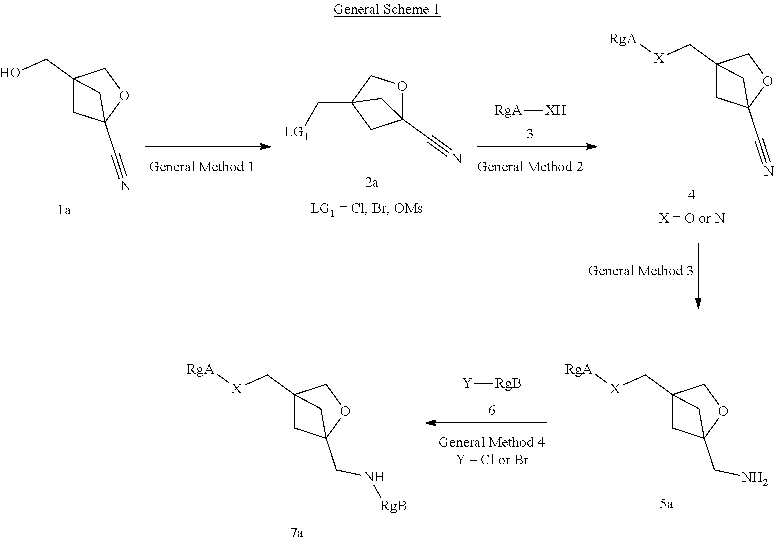

Alcohols such as 1a are typically reacted to form a suitable leaving group, such as halide or mesylate and can be generated using conditions well known in the art such as, for example; chlorination via a mesylate, bromination with PBr₃, or bromination with CBr₄ and PPh₃, using a suitable solvent such as DCM, THF or CCl₄(General Method 1), to give compound 2a. Compound 2a is subsequently reacted with an alcohol or amine 3 under typical alkylation conditions (General Method 2, eg KO^{t}Bu or NaH in DMF or Cs₂CO₃ or K₂CO₃ in MeCN or DMSO, with heating as necessary). The nitrile 4 can be reduced to amine 5a under a variety of standard literature conditions well known in the art (General Method 3); for example under hydrogenation in the presence of Raney Ni, alternatively hydrogenation in the presence of Pd/C, or alternatively with NiCl₂ and sodium borohydride (NaBH₄) in the presence of Boc₂O, or alternatively with borane. The amine 5a is reacted with aryl bromide or chloride 6 under Buchwald coupling conditions (General Method 4). This Buchwald coupling is carried out for example using a Buchwald pre catalyst, such as BrettPhos Pd G3, BrettPhos Pd G4 or Pd-PEPPSI-Ipent, in the presence of a base such a sodium tert-butoxide, Cs₂CO₃, or lithium hexamethyldisilazide (LiHMDS), in a solvent such as 1,4-dioxane or THF. The aryl bromide or chloride 6 can be prepared from readily available starting materials using methods known in the art, or as described herein. Depending on the identity of RgB, a deprotection step (detailed below) may be required to obtain the example compound.

Alternatively, for example as shown in General Scheme 2, where material is available as the amine, for example compound 1b, a similar synthetic sequence can be applied.

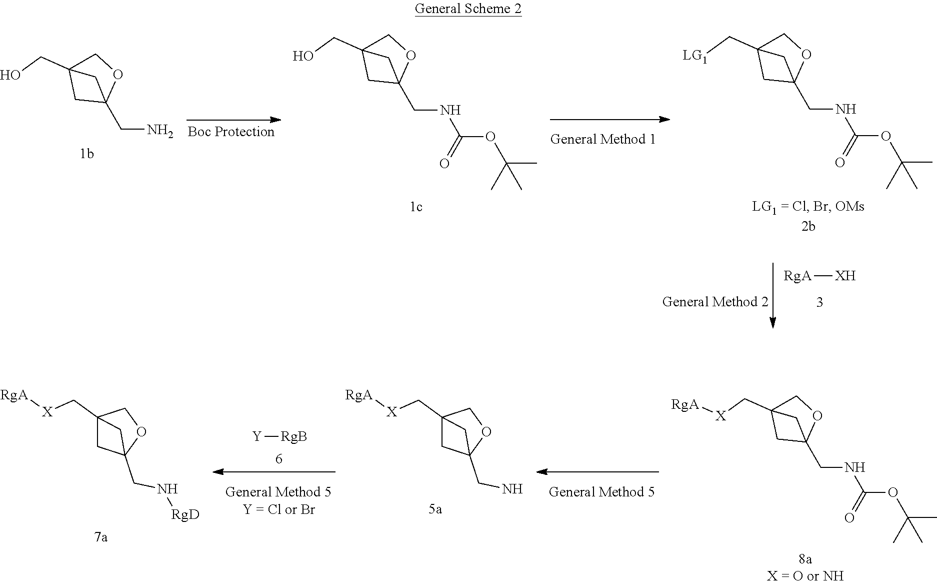

The amine is protected, for example with a tert-butoxy carbamate protecting group, using standard conditions such as TEA and Boc₂O in DCM or THF. Following formation of a leaving group and alkylation, the tert-butoxy carbamate protecting group is removed from compound 8a using standard conditions such as TFA, or HCl in 1,4-dioxane (General Method 5). Finally, Buchwald coupling (General Method 4) completes the route.

The order of steps can also be reversed, for example as shown in General Scheme 3.

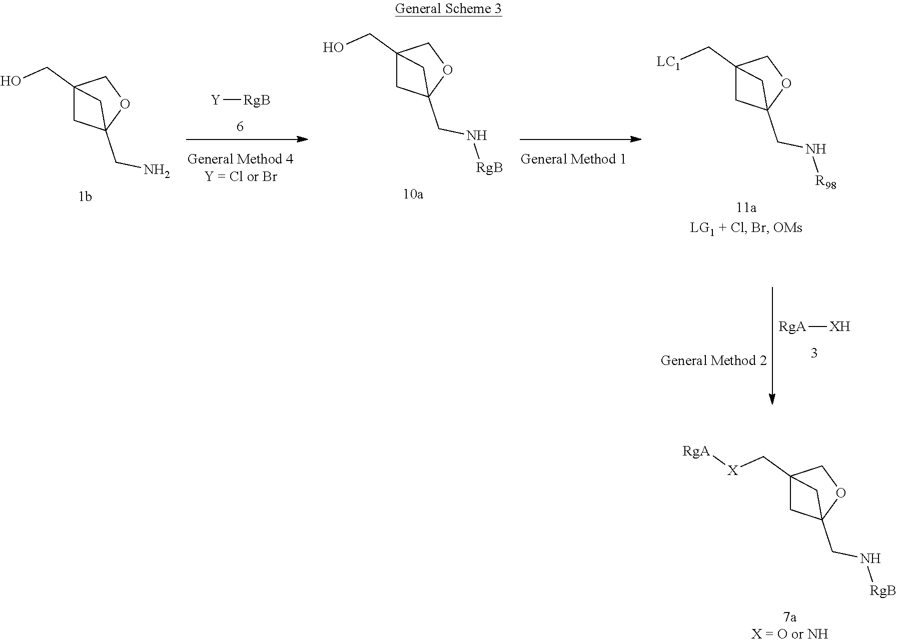

An amine such as 1b can undergo a Buchwald (General Method 4), followed by formation of a leaving group (General Method 1) and finally alkylation with compound 3 (General Method 2).

Furthermore, the Buchwald coupling (General Method 4), is also possible with alcohols. This requires a suitable protecting group strategy as shown in General Schemes 4 and 5.

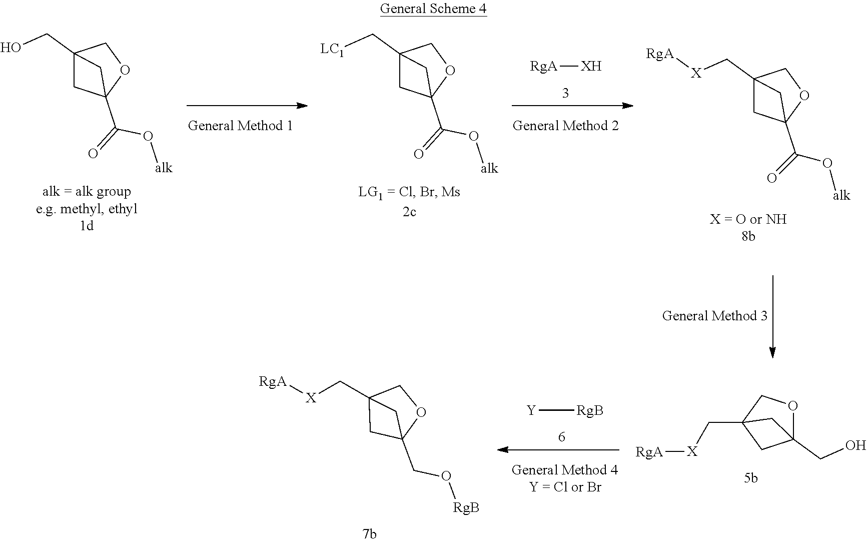

In General Scheme 4, a protected alcohol is in the form of an ester such as 1d. Following the usual sequence of steps, formation of a leaving group and alkylation, the ester 8b is reduced using standard conditions such as LiAIH₄ (General Method 3) to reveal the alcohol. Finally, Buchwald coupling (General Method 4) of alcohol 5b completes the route. This typically requires an elevated temperature, for example 90 °C or above and in some cases alternative catalysts, such as RockPhos Pd G3, are more suitable.

Again, the order of steps can also be reversed, for example as shown in General Scheme 5.

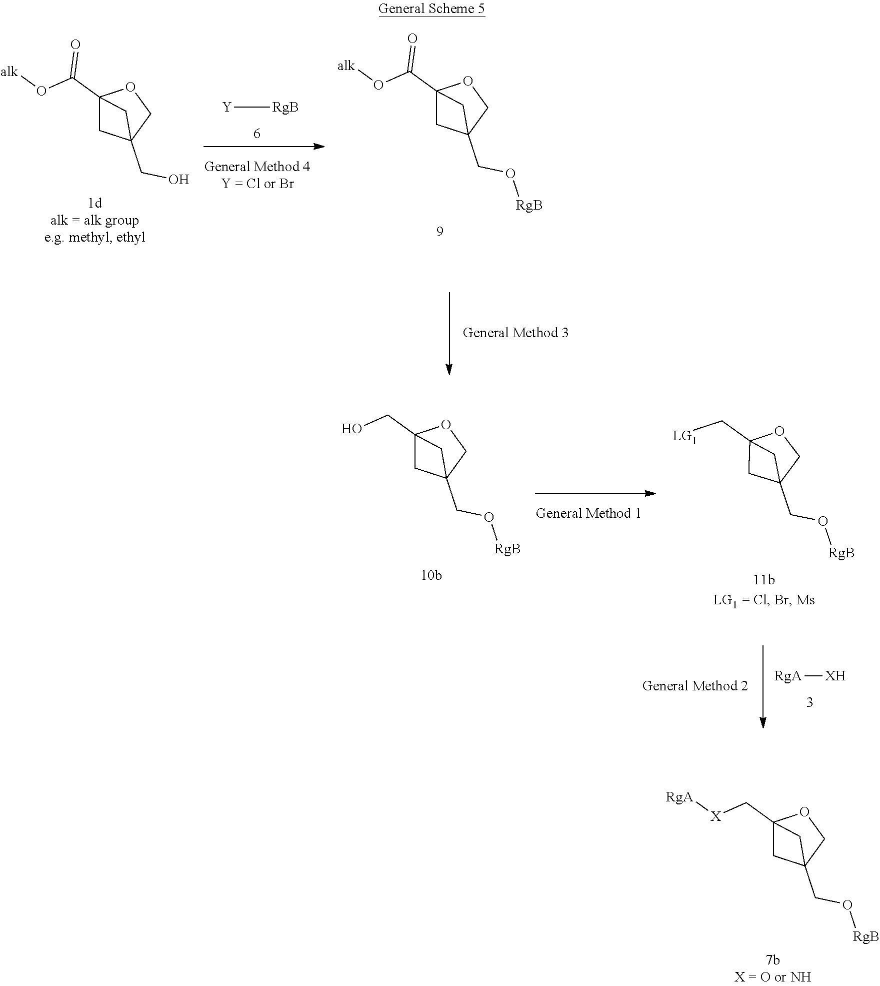

The alcohol such as 1d can undergo a Buchwald (General Method 4), followed by reduction of the ester, formation of a leaving group (General Method 1) and finally alkylation with compound 3 (General Method 2).

In examples, where RgA contains a bicyclic aromatic, it can be partially saturated during the synthesis, as shown in General Scheme 6.

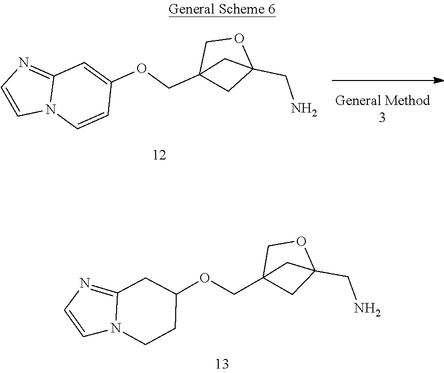 [SMILES: NCC12CC(COc3ccn4ccnc4c3)(CO1)C2 ; NCC12CC(COC3CCn4ccnc4C3)(CO1)C2]

Typically the aromatic ring such as 12 is subjected to hydrogenation at elevated temperature (General Method 3) using H₂, 10% Pd/C to give compound 13. This transformation can take place on the free or protected amine.

In example compounds described herein containing a primary or secondary amine, a protecting group strategy may be required. Alternative protecting groups can be used with different deprotection conditions, that is, an orthogonal protecting group strategy can be applied. General Schemes 7-12 outline possible protecting group strategies that may be used for the synthesis of the examples.

For example, for compounds defined herein containing a 6,6 ring system, as shown in General Scheme 7, a protected amine can be installed. Initially the pyridone 14 may be chlorinated, typically using phosphorous oxychloride (General method 6). The resulting chloride 15 may be reacted with 2,4-dimethoxybenzylamine using General Method 7, for example using basic conditions such as K₂CO₃, Cs₂CO₃, or pyridine in a solvent such as NMP or MeCN, either thermally and/or under microwave conditions. RgT refers to various substituents as required by the example compounds.

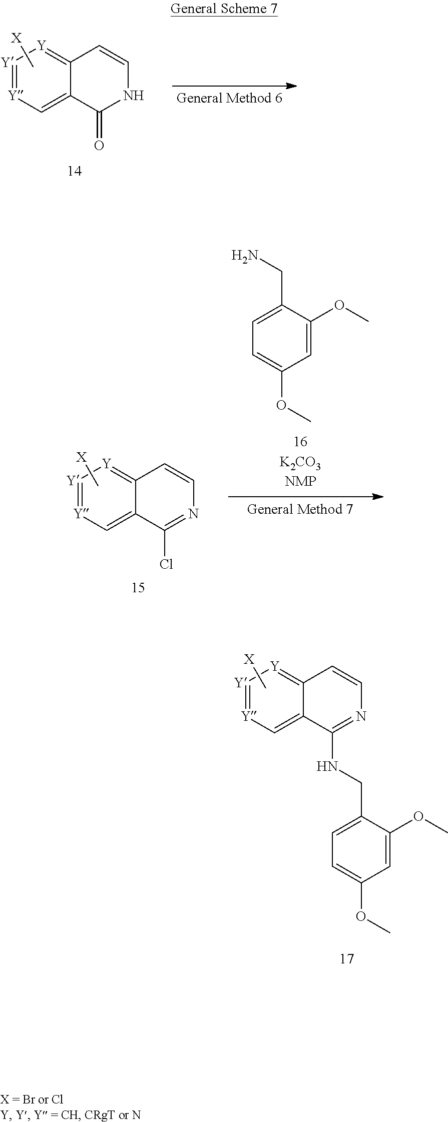

Typically, at the end of the synthetic sequence, the 2,4-dimethoxybenzyl protecting group is removed using TFA at 50 °C, either undiluted or diluted in DCM (General Scheme 8, General Method 8). RgT, RjA and RjB refer to various substituents as required by the examples.

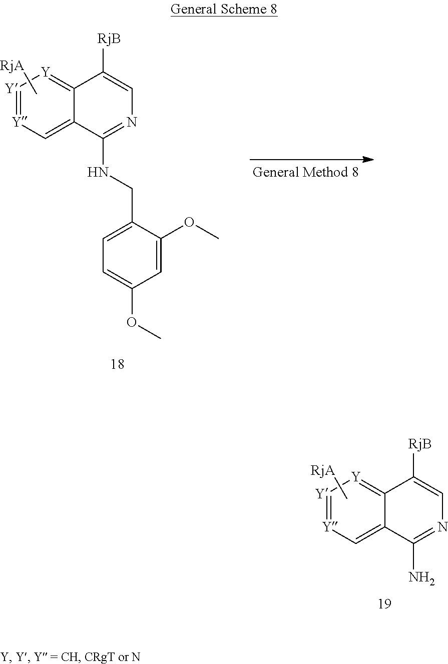

Alternatively, when starting materials are available with the amine already installed, a carbamate protecting group can be used. For example, as outlined in General Scheme 9, the amine is reacted with methyl chloroformate under basic conditions with organic bases such as TEA or DIPEA in a solvent such as DCM to afford the methyl carbamate 21 (General Method 9). RjC refers to various substituents as required by the examples.

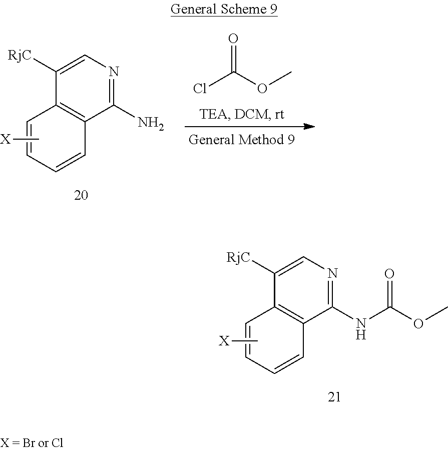

Typically, at the end of the synthetic sequence, the methyl carbamate protecting group is deprotected using basic conditions, such as KOH or LiOH in solvents such as 1,4-dioxane, MeCN, THF and optionally 10% water, at elevated temperature, typically 50 °C (General Scheme 10, General Method 10). RjC and RjD refer to various substituents as required by the examples.

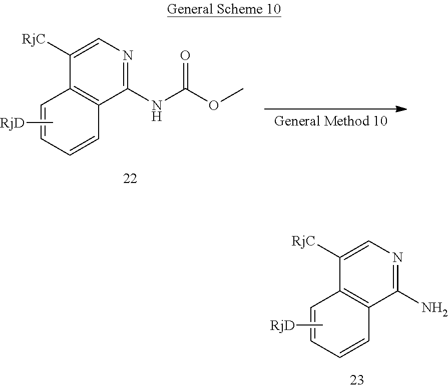

Another protecting group that may be used where example compounds described herein contain a 6,6 ring system is boc.

Where, for example, example compounds described herein contain a 5,6 ring system, SEM, boc and sulfonyl protecting groups may typically be used. Protecting groups may subsequently be deprotected using standard literature procedures, for example those described by T. W. Greene and P. G. M. Wuts in “Protective groups in organic chemistry” John Wiley and Sons, 4^{th} Edition, 2006.

An example of the installation of a SEM protecting group is shown in General Scheme 11 whereby the indole 24 is treated with a base such as NaH in a solvent such as DMF, followed by addition of SEM-Cl (General Method 2).

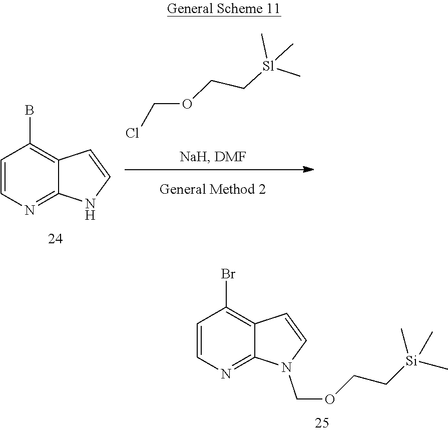

Typically, at the end of the synthetic sequence the SEM protecting group is deprotected using acidic conditions such as TFA in DCM (General Scheme 12, General Method 11). RjD refers to various substituents as required by the examples.

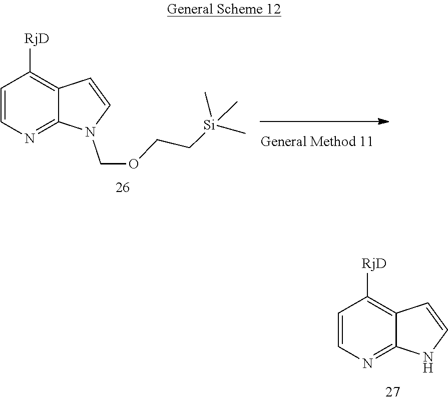

In examples where RgA contains an aromatic or heteroaromatic bicycle they can be synthesised using procedures described in the literature and, for example, as depicted in General Scheme 13.

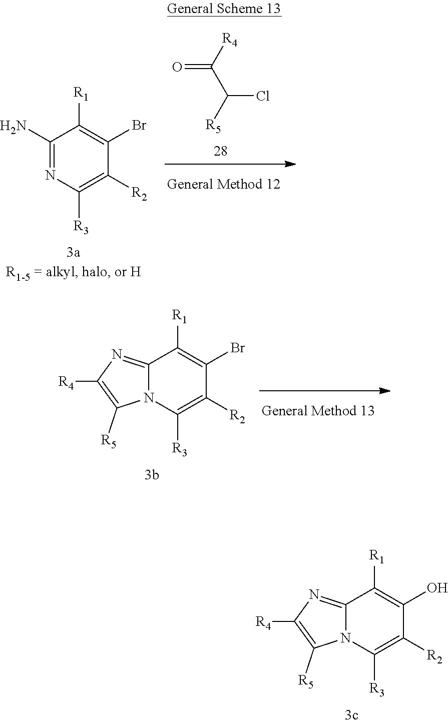

Amino pyridines such as 3a can be reacted with chloromethyl aldehydes and ketones 28 by refluxing in a solvent such as ethanol (General Method 12). Hydroxylation of the aryl bromide 3b to the corresponding aryl alcohol 3c is typically completed in a one-pot tandem process via Miyaura borylation (Palladium(0)-Catalyzed Cross-Coupling Reaction of Alkoxydiboron with Haloarenes: A Direct Procedure for Arylboronic Esters. T. Ishiyama, M. Murata, N. Miyaura, J. Org. Chem., 1995, 60, 7508-7510) and subsequent hydroxylation using hydrogen peroxide (General Method 13).

Other examples herein may be based on an azabicyclohexyl central core group, as shown in General Schemes 14-17. These General Schemes may also be applied to other central core groups such as bicyclopentyl, oxabicyclohexyl and oxabicycloheptyl. The starting materials may be readily available from commercial sources or may be known in the literature. Alternatively, some prior manipulation may be required, for example, as shown in General Scheme 14, a carboxylic acid such as 1e can be esterified using methods generally known in the art, for example via formation of the acid chloride in an appropriate alcohol solvent such as methanol (General Method 14).

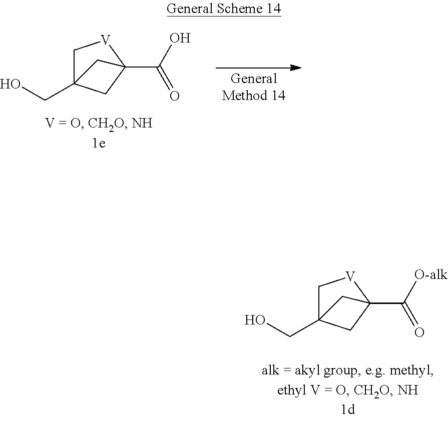

When azabicyclohexyl rings are used they may be protected with a suitable nitrogen protecting group such as Boc or Cbz (General Scheme 15). Alternatively, the nitrogen can be reacted using methods generally known in the art and detailed herein below, to provide amide, urea, thiourea, alkyl, heteroaryl and sulfonamide analogues as shown in General Schemes 15 and 17.

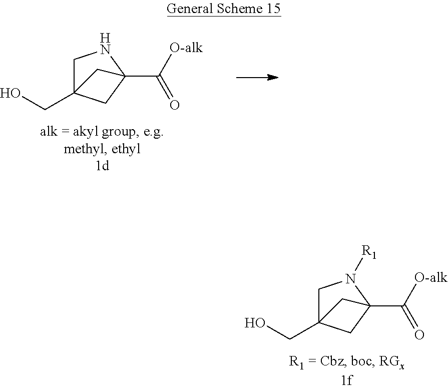

As outlined in previous schemes and as repeated in General Scheme 16, RgA can be installed by transforming the alcohol 1d to a leaving group, such as a mesylate or halide 7b and reacting with aryl alcohols (General Method 2). Alternatively, the alcohol 1d can be directly reacted with phenols under Mitsunobu conditions (General Method 15) using standard conditions such as DIAD and triphenylphosphine in THF. The alcohol 1d can also be coupled with aryl halides under SNAr in the presence of NaH (General Method 7a), or under Buchwald conditions (General Method 4), typically using RockPhos Pd G3 as the catalyst and elevating the temperature to 100 °C. The ester 8b is then reduced to alcohol 5c under conditions well known in the art such as lithium borohydride (LiBH₄) or lithium aluminium hydride (LiAIH₄) or lithium triethylborohydride (LiEt₃BH) (General Method 3b) depending on other functionality contained in the compound. Oxidation to aldehyde 5d is typically achieved using a reagent such as Dess Martin periodinane (General Method 16). Finally, reductive amination (General Method 17) with amine 6a completes the route. Alternatively (not shown in the Schemes) the alcohol 5c can be transformed to an amine using methods generally known in the art, such as via conversion to a leaving group (General Method 1) then via an azide or Gabriel synthesis to provide the primary amine and finally completing the route via a Buchwald coupling (General Method 4) as shown previously in General Scheme 1.

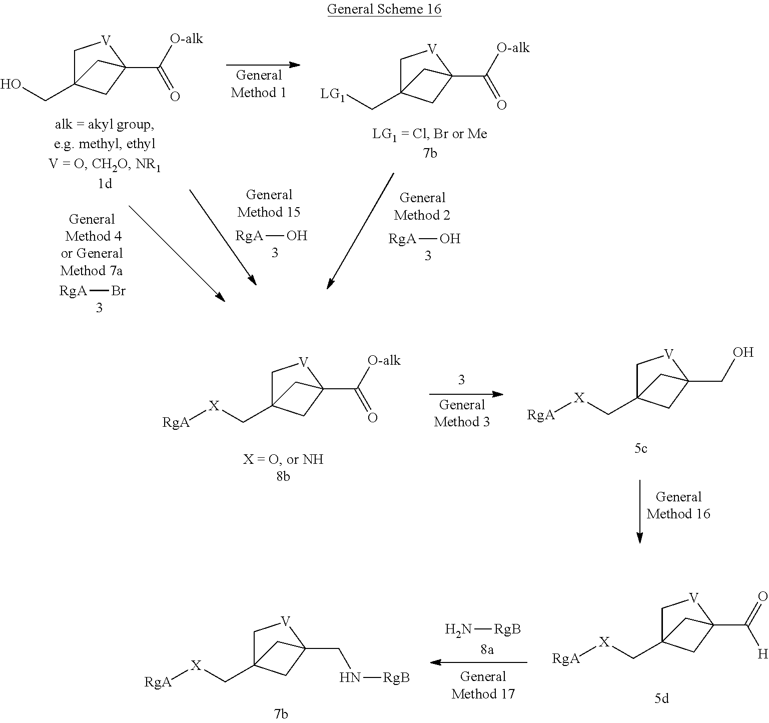

When azabicyclohexyl rings are used, in which the nitrogen may be protected by a protecting group (i.e. 7b where V═NR₁), a late stage functionalisation is possible, as shown in General Scheme 17. The nitrogen protecting group is removed using the appropriate conditions (for example boc deprotection via General Method 5, or Cbz deprotection via General Method 3e, 2,4-dimethoxybenzyl deprotection via General Method 8, or SEM deprotection via General Method 11). Manipulation of 7c using chemistry well known in the art can provide a variety of compound classes. For example, reductive alkylation with aldehydes (General Method 17) can give alkylated compounds such as 29a. Cross-coupling reactions with aryl halides, such as Buchwald reactions (General Method 4) or SNAr alkylation (General Method 7) can install aromatic groups to give 29f. Reaction of 7c with carboxylic acids or acid chlorides can be used to provide amides 29b (General Method 18) or reaction with sulphonyl chlorides (General Method 19) can provide sulphonamides 29c. Reaction with CDI and alcohols can provide carbamates 29d (General Method 20). Ureas such as 29e can be formed by reaction of 7c with isocyanates, or by reacting with CDI or triphosgene and amines (General Method 21).

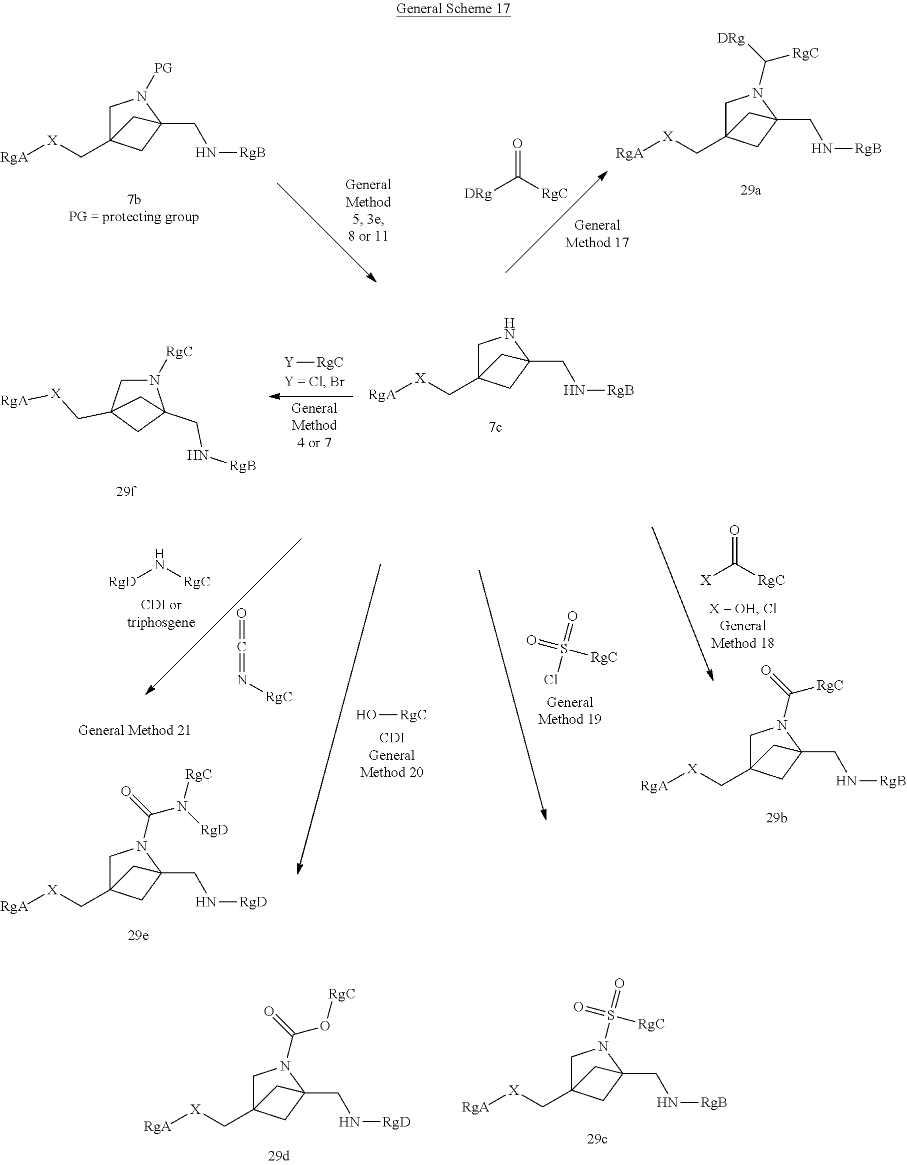

The routes described are also applicable to azabicyclohexyl isomers and the order of steps may vary depending on starting material availability or as chemical functionality requires.

### Synthesis of Intermediates

### Intermediate 1

### Methyl (6-bromoisoquinolin-1-yl)carbamate

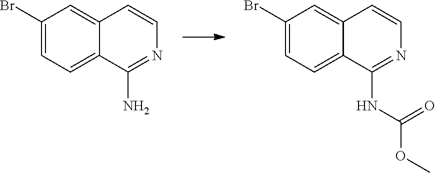 [SMILES: Nc1nccc2cc(Br)ccc12 ; COC(=O)Nc1nccc2cc(Br)ccc12]

Following General Method 9, 6-bromoisoquinolin-1-amine (1.50 g, 6.72 mmol) was protected. The crude was suspended in water (100 mL) and stirred for 30 min before being collected by filtration and dried in the vacuum oven overnight to give the product (1.12 g, 44% yield).

[M+H]⁺=281.1

¹H NMR (500 MHz, DMSO-d6) 3.70 (3H, s), 7.58-7.72 (1H, m), 7.79 (1H, d, J=9.0, 2.0 Hz), 8.04 (1H, d, J=9.1 Hz), 8.25-8.30 (1H, m), 8.33 (1H, d, J=5.8 Hz), 10.18 (1H, s)

### Intermediate 2

### Methyl (6-bromo-4-chloroisoquinolin-1-yl)carbamate

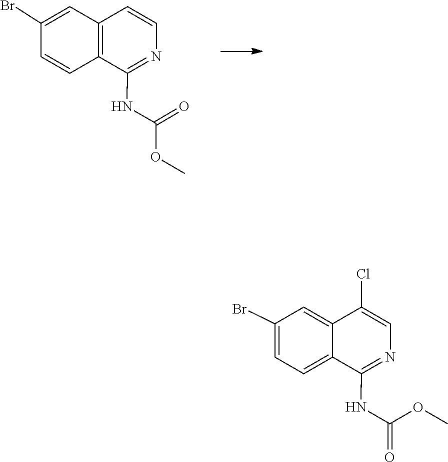 [SMILES: COC(=O)Nc1nccc2cc(Br)ccc12 ; COC(=O)Nc1ncc(Cl)c2cc(Br)ccc12]

Methyl N-(6-bromo-1-isoquinolyl)carbamate (100 mg, 0.36 mmol) was dissolved in chloroform (5 mL), NCS (52 mg, 0.39 mmol) was added and the reaction stirred at reflux for 18 h. To the reaction was added sat. NaHCO₃ (aq.) (30 mL) and it was washed with DCM (30 mL), dried (Na₂SO₄) and concentrated. The crude product was purified by flash chromatography (Silica, 0-80% EtOAc in Pet. Ether) to give the product (74 mg, 59% yield).

[M+H]⁺=316.8/318.7

¹H NMR (CDCl₃, 400 MHz) δ 3.84 (3H, s), 7.36 (1H, s), 7.75 (1H, dd, J=9.0, 1.9 Hz), 7.93 (1H, d, J=9.0 Hz), 8.37 (2H, d, J=4.9 Hz)

### Intermediate 3

### Methyl (5-bromoisoquinolin-1-yl)carbamate

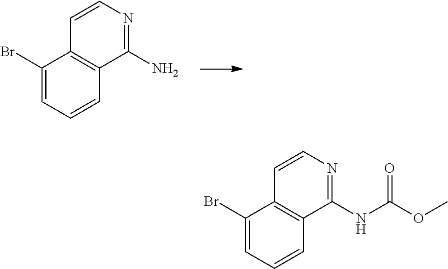 [SMILES: Nc1nccc2c(Br)cccc12 ; COC(=O)Nc1nccc2c(Br)cccc12]

Following General Method 9, 5-bromoisoquinolin-1-amine (1.12 g, 5.02 mmol) was protected. The product was dried under high vacuum to yield (838 mg, 56% yield).

[M+H]⁺=281.1

### Intermediate 4

### 5-Bromo-N-(2,4-dimethoxybenzyl)isoquinolin-1-amine

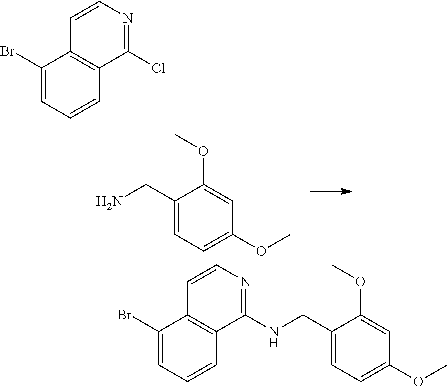

Following a variation of General Method 7d, to a solution of 5-bromo-1-chloroisoquinoline (0.5 g, 2.06 mmol) in pyridine (3 mL), was added 2,4-dimethoxybenzylamine (0.69 g, 4.12 mmol). The reaction was heated at 150 °C in a CEM Microwave for 60 min. The mixture was diluted with DCM (20 mL) and water (20 mL). The aqueous layer was re-extracted with DCM (3×10 mL) and the combined organics were washed with brine (20 mL). The organic layer was dried (Na₂SO₄), filtered and concentrated. Purification by flash chromatography (Silica, 20-50% EtOAc in Pet. Ether) afforded the product (276 mg, 50% yield).

[M+H]⁺=373.0/375.0

¹H NMR (DMSO-d₆, 400 MHz) δ3.71 (3H, d, J=2.6 Hz), 3.82 (3H, d, J=2.8 Hz), 4.62 (2H, d, J=5.4 Hz), 6.41 (1H, dd, J=8.5, 2.5 Hz), 6.56 (1H, d, J=2.6 Hz), 6.94-7.14 (2H, m), 7.42 (1H, t, J=8.0 Hz), 7.96 (3H, ddd, J=16.4, 7.1, 3.2 Hz), 8.38 (1H, d, J=8.2 Hz)

### Intermediate 5

### N1-(2,4-Dimethoxybenzyl)isoquinoline-1,5-diamine

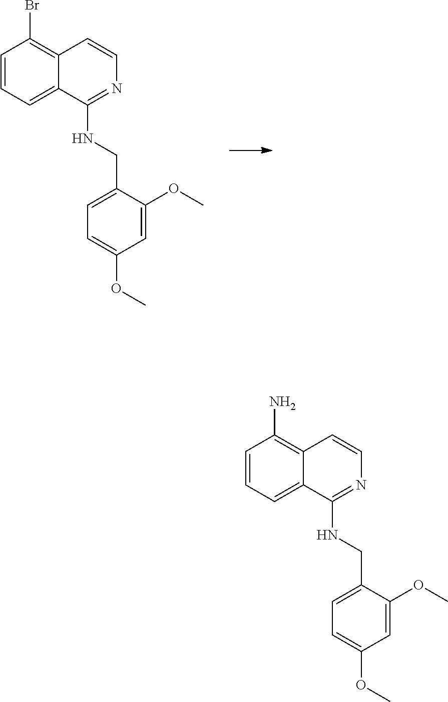 [SMILES: COc1ccc(CNc2nccc3c(Br)cccc23)c(OC)c1 ; COc1ccc(CNc2nccc3c(N)cccc23)c(OC)c1]

A mixture of 5-bromo-N-(2,4-dimethoxybenzyl)isoquinolin-1-amine (746 mg, 2.0 mmol), 2,2,2-trifluoroacetamide (339 mg, 3.0 mmol), CuI₂ (38 mg, 0.2 mmol), K₂CO₃ (553 mg, 4.0 mmol) and N,N′-dimethylethylenediamine (35 mg, 0.4 mmol) were combined in a reaction vial. Anhydrous 1,4-dioxane (7 mL) was added, and the suspension purged with N₂, before being capped and then heated to 75 °C for 24 h. The reaction was recharged with 2,2,2-trifluoroacetamide (339 mg, 3.0 mmol), CuI₂ (38 mg, 0.2 mmol), K₂CO₃ (553 mg, 4.0 mmol) and N,N′-dimethylethylenediamine (35 mg, 0.4 mmol). The mixture was degassed with N₂ and heated at 70 °C for 18 h. Water was added (3 mL) and the reaction heated at 80 °C for 6 h. The mixture was partitioned between EtOAc (30 mL) and water (10 mL). The aqueous layer was extracted with further EtOAc (2×30 mL) and the combined organics were washed with brine (50 mL), dried (Na₂SO₄), filtered and concentrated. Purification by flash chromatography (Silica, 0-100% EtOAc/DCM) afforded the product (492 mg, 72% yield).

[M+H]⁺ 310.1

¹H NMR (CDCl₃, 400 MHz) δ 3.79 (3H, s), 3.85 (3H, s), 4.10 (2H, br s), 4.72 (2H, d, J=5.3 Hz), 5.58-5.67 (1H, m), 6.44 (1H, dd, J=8.2, 2.4 Hz), 6.49 (1H, d, J=2.4 Hz), 6.78-6.86 (2H, m), 7.12 (1H, dt, J=8.4, 1.1 Hz), 7.21 (1H, dd, J=8.4, 7.4 Hz), 7.30 (1H, d, J=8.2 Hz), 8.01 (1H, d, J=6.1 Hz)

### Intermediate 6

### 4-Bromo-1-((2-(trimethylsilyl)ethoxy)methyl)-1H-pyrrolo[2,3-b]pyridine

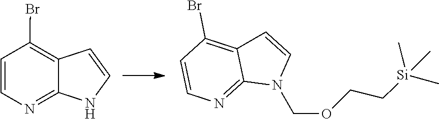 [SMILES: Brc1ccnc2[nH]ccc12 ; C[Si](C)(C)CCOCn1ccc2c(Br)ccnc21]

Following General Method 2a, 4-bromo-1H-pyrrolo[2,3-b]pyridine (500 mg, 2.54 mmol) was reacted with SEM-Cl (494 μL, 2.79 mmol) for 2 h. The reaction mixture was quenched by the careful addition of water (10 mL) followed by repeat extraction with EtOAc. (3×20 mL). The combined organic layers were then washed with sat. NaHCO₃ (aq) (30 mL), water (30 mL) and brine (30 mL), before being dried (MgSO4), filtered and concentrated. Purification by flash chromatography (Silica, 0-100% EtOAc in iso-hexane) afforded the product (500 mg, 57% yield).

[M+H]⁺=327.2

¹H NMR (500 MHz, DMSO-d6) δ −0.11 (s, 9H), 0.75-0.86 (m, 2H), 3.43-3.56 (m, 2H), 5.63 (s, 2H), 6.52 (d, J=3.6 Hz, 1H), 7.43 (d, J=5.1 Hz, 1H), 7.79 (d, J=3.6 Hz, 1H) 8.16 (d, J 5.1 Hz, 1H)

### Intermediate 7

### 5-Bromo-3-chloro-1-((2-(trimethylsilyl)ethoxy)methyl)-1H-pyrrolo[2,3-b]pyridine

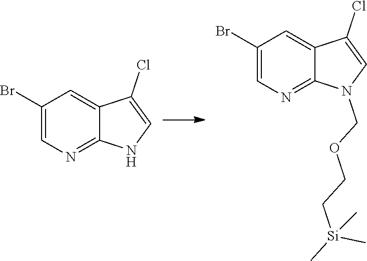 [SMILES: Clc1c[nH]c2ncc(Br)cc12 ; C[Si](C)(C)CCOCn1cc(Cl)c2cc(Br)cnc21]

Following General Method 2a, 5-bromo-3-chloro-1H-pyrrolo[2,3-b]pyridine (480 mg, 2.07 mmol) was reacted with SEM-Cl (0.4 mL, 2.28 mmol) for 2 h. The reaction was quenched with water (2 mL) and diluted with EtOAc (40 mL). The organic layer was washed with water (20 mL), 1M HCl (aq) (20 mL), 1:1 water/brine (20 mL) and brine (20 mL), dried (MgSO₄), filtered and concentrated. Purification by flash chromatography (Silica, 0-100% EtOAc in iso-hexane) afforded the product (485 mg, 60% yield).

[M+H]⁺=363.0

¹H NMR (500 MHz, DMSO-d6) δ-0.10 (9H, s), 0.81 (2H, t, J=7.9 Hz), 3.51 (2H, t, J=7.9 Hz), 5.60 (2H, s), 7.98-8.01 (1H, m), 8.20-8.24 (1H, m), 8.44-8.47 (1H, m)

### Intermediate 8

### 5-Bromo-N-(2,4-dimethoxybenzyl)-4-fluoroisoquinolin-1-amine

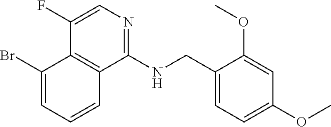 [SMILES: COc1ccc(CNc2ncc(F)c3c(Br)cccc23)c(OC)c1]

### 5-Bromo-1-chloro-4-fluoroisoquinoline

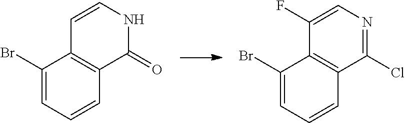 [SMILES: O=c1[nH]ccc2c(Br)cccc12 ; Fc1cnc(Cl)c2cccc(Br)c12]

A solution of 5-bromo-2H-isoquinolin-1-one (9.0 g, 40.2 mmol) and Selectfluor® (17.1 g, 48.2 mmol) in MeCN (120 mL) and MeOH (120 mL) were heated at 50 °C for 3 h. The reaction mixture was concentrated and reacted using General Method 6, in 1,2-dichloroethane (200 mL) using benzyltriethylammonium chloride (915 mg, 4.0 mmol) and phosphorus oxychloride (45 mL) at 90 °C for 24 h. The reaction mixture was concentrated, and the residue partitioned between DCM (500 mL) and water (500 mL). The organic layer was washed with water (300 mL) and brine (300 mL), dried (MgSO₄) and evaporated. The crude was purified by flash chromatography (Silica, 0-30% EtOAc in Pet. Ether) to give the product (5.70 g, 55% yield).

[M+H]⁺=261.9

¹H NMR (500 MHz, CDCl₃) δ 8.39-8.33 (m, 1H), 8.23 (d, J=4.0 Hz, 1H), 8.12-8.06 (m, 1H), 7.57 (t, J=8.0 Hz, 1H)

### 5-Bromo-N-(2,4-dimethoxybenzyl)-4-fluoroisoquinolin-1-amine

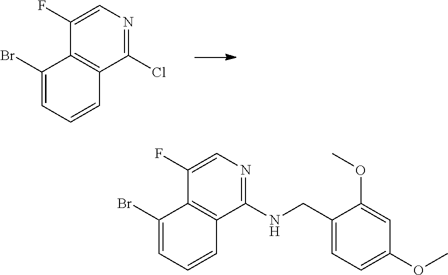 [SMILES: Fc1cnc(Cl)c2cccc(Br)c12 ; COc1ccc(CNc2ncc(F)c3c(Br)cccc23)c(OC)c1]

Following General Method 7d, 5-bromo-1-chloro-4-fluoroisoquinoline (5.70 g, 21.9 mmol) was reacted with 2,4-dimethoxybenzylamine (4.93 mL, 32.8 mmol) in 1-methyl-2-pyrrolidinone (80 mL) at 100 °C for 48 h. The crude product was purified by flash chromatography (Silica, 0-30% EtOAc in Pet. Ether) to give the product (1.05 g, 12% yield).

¹H NMR (500 MHz, DMSO) δ 8.43 (dd, J=8.1, 2.3 Hz, 1H), 8.06 (dd, J=7.6, 0.9 Hz, 1H), 7.89 (d, J=5.1 Hz, 1H), 7.81 (t, J=5.6 Hz, 1H), 7.49 (t, J=8.0 Hz, 1H), 7.05 (d, J=8.3 Hz, 1H), 6.56 (d, J=2.4 Hz, 1H), 6.41 (dd, J=8.4, 2.4 Hz, 1H), 4.57 (d, J=5.5 Hz, 2H), 3.82 (s, 3H), 3.72 (s, 3H)

¹⁹F NMR (471 MHz, DMSO) δ-149.9(s)

[M−H]⁻=389.2

### Intermediate 9

### 1-(Benzenesulfonyl)-4-bromo-2-chloropyrrolo[2,3-b]pyridine

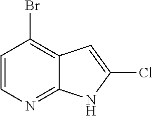 [SMILES: Clc1cc2c(Br)ccnc2[nH]1]

### 1-(Benzenesulfonyl-4-bromopyrrolol[2,3-b]pyridine

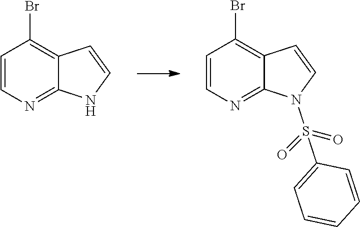

To a solution of 4-bromo-1H-pyrrolo[2,3-b]pyridine (5.00 g, 25.4 mmol) in DCM (130 mL) was added benzenesulfonyl chloride (4.86 mL, 38.1 mmol), 4-dimethylaminopyridine (310 mg, 2.54 mmol) and TEA (10.6 mL, 76.1 mmol). The reaction mixture was stirred at rt for 2 h. Upon completion the reaction mixture was concentrated. The product was suspended in DCM (50 mL) and concentrated onto silica. The material was purified via flash chromatography (Silica, 0-50% EtOAc in Pet. Ether) to afford the product (8.39 g, 98% yield).

[M+H]⁺=338.9

### 1-(Benzenesulfonyl)-4-bromo-2-chloropyrrolo[2,3-b]pyridine

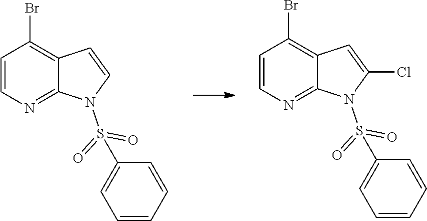 [SMILES: O=S(=O)(c1ccccc1)n1ccc2c(Br)ccnc21 ; O=S(=O)(c1ccccc1)n1c(Cl)cc2c(Br)ccnc21]

1-(Benzenesulfonyl)-4-bromopyrrolo[2,3-b]pyridine (1.00 g, 2.97 mmol) in anhydrous THF (16 mL) was cooled to −41 °C and lithium diisopropylamide (2M in THF) (356 mL, 7.12 mmol) was added slowly. The resulting precipitate was stirred for 30 min at −41 °C before benzenesulfonyl chloride (757 μL, 5.93 mmol) was added. The reaction mixture was stirred for 2.5 h at −41 °C The reaction mixture was quenched with water (10 mL) and diluted with EtOAc (20 mL). The layers were separated, and the aqueous layer was back extracted with EtOAc (2×20 mL). The organic layers were combined, washed with brine (10 mL), dried (MgSO₄), filtered and concentrated. Purification by flash chromatography (Silica, 0-60% EtOAc in Pet. Ether) afforded the product (895 mg, 49% yield).

[M+H]⁺=372.9

### 1-(Benzenesulfonyl)-4-bromo-2-chloropyrrolo[2,3-b]pyridine

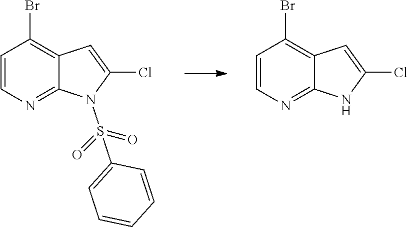

To a solution of 1-(Benzenesulfonyl)-4-bromo-2-chloropyrrolo[2,3-b]pyridine (895 mg, 1.44 mmol) in 1,4-dioxane (7 mL) was added NaOtBu (324 mg, 2.89 mmol). The reaction mixture was stirred at 80 °C for 2 h, diluted with EtOAc (10 mL) and washed with brine (10 mL). Layers were separated and the organic layer was dried (MgSO₄), filtered and concentrated. Purification by flash chromatography (Silica, 0-25% EtOAc in Pet. Ether) afforded the product (295 mg, 88% yield).

[M+H]⁺=232.9

¹H NMR (CDCl₃, 400 MHz) δ 6.47 (1H, s), 7.32 (1H, d, J=5.3 Hz), 8.11 (1H, d, J=5.3 Hz) NH proton not observed

### Intermediate 10

### 3-Methylimidazo[1,2-a]pyridin-6-ol

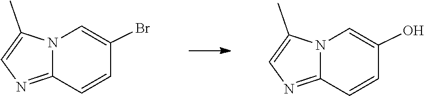

According to a modification of General Method 13, 6-bromo-3-methylimidazo[1,2-a]pyridine (525 mg, 2.49 mmol) was reacted with B₂Pin₂ (1.42 g, 5.6 mmol) at 100 °C for 5 h. The mixture was cooled to rt before AcOH (285 μL, 4.97 mmol) and water (1 mL) were added. The mixture was stirred for 15 min before a solution of H₂O₂ in water (30% w/w, 508 μL, 4.97 mmol) was added dropwise at 0 °C The mixture was stirred for 90 min at rt, quenched with solid Na₂S2O₃ (5 mg), filtered through Celite® and concentrated. The product was passed directly through SCX and washed with MeOH. The product was eluted with a solution of 1M NH₃ in MeOH and concentrated to afford the product (160 mg, 33% yield).

[M+H]⁺=149.1

¹H NMR (DMSO, 400 MHz) δ 2.36 (3H, s), 6.92 (1H, dd, J=9.6, 2.3 Hz), 7.25 (1H, s), 7.40 (1H, d, J=9.5 Hz), 7.59 (1H, d, J=1.9 Hz), 9.45 (1H, s)

### Intermediate 11

### 7-hydroxy-2,3-dihydroindolizin-5(1H)-one

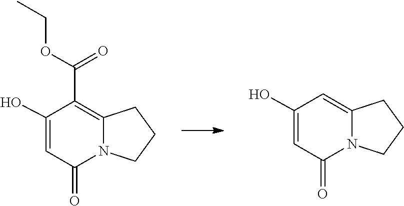 [SMILES: CCOC(=O)c1c(O)cc(=O)n2c1CCC2 ; O=c1cc(O)cc2n1CCC2]

Ethyl 7-hydroxy-5-oxo-2,3-dihydro-1H-indolizine-8-carboxylate (125 mg, 0.56 mmol) was dissolved in 12M HCl (aq) (1.87 mL, 22.4 mmol) and stirred at reflux for 18 h. The solution was cooled to rt, then azeotroped with Et₂O (3×5 mL) and concentrated to provide the product (123 mg, 87% yield) which was used for the next step without further purification.

[M+H]⁺=152.1

### Intermediate 12

### Methyl 4-(hydroxymethyl)-2-azabicyclo[2.1.1]hexane-1-carboxylate; hydrochloride

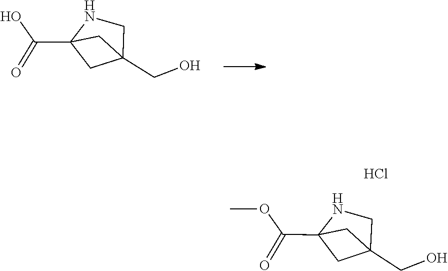

The product was prepared from 4-(hydroxymethyl)-2-azabicyclo[2.1.1]hexane-1-carboxylic acid (CAS 1522098-73-2, Tetrahedron Letters 55 (2014) 466-468) following the procedure described in ChemistrySelect 2019, 4, 4933-493.

### Intermediate 13

### Methyl 5-(hydroxymethyl)-2-oxabicyclo[3.1.1]heptane-1-carboxylate

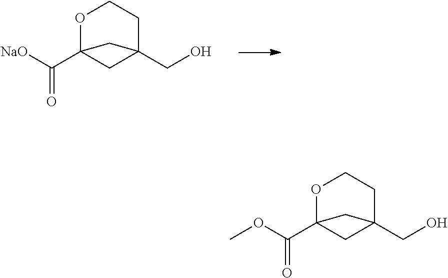

Following General Method 14, 5-(hydroxymethyl)-2-oxabicyclo[3.1.1]heptane-1-carboxylic acid sodium salt (600 mg, 2.8 mmol) was reacted for 22 h. The reaction mixture was diluted with DCM (40 mL) and washed with water (20 mL). The aq layer was extracted with DCM (30 mL). The combined organic fractions were dried (Na₂SO₄) and concentrated to afford the product (409 mg, 71% yield).

[M+H]⁺=187.1

¹H NMR (CDCl₃, 400 MHz) δ 1.94-2.05 (4H, m), 2.33 (2H, ddt, J=7.2, 2.3, 1.0 Hz), 3.54 (2H, s), 3.77 (3H, s), 4.19-4.27 (2H, m)

### Methyl 5-(methylsulfonyloxymethyl)-2-oxa bicyclo[3.1.1]heptane-1-carboxylate

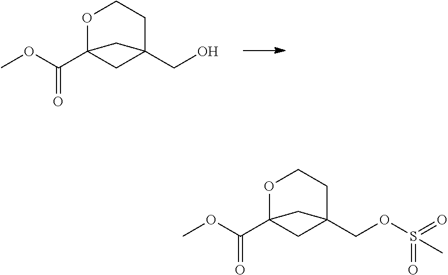

According to General Method 1a, methyl 5-(hydroxymethyl)-2-oxabicyclo[3.1.1]heptane-1-carboxylate (404 mg, 1.95 mmol) was reacted with MsCl (227 μL, 2.93 mmol for 22 h. Aqueous work up afforded the product (372 mg, 58% yield).

¹H NMR (CDCl₃, 400 MHz) δ 2.05-2.13 (4H, m), 2.35-2.44 (2H, m), 3.05 (3H, s), 3.78 (3H, s), 4.12 (2H, s), 4.19-4.26 (2H, m)

### Methyl 5-[(1,3-dioxoisoindol-2-yl)methyl]-2-oxabicyclo[3.1.1]heptane-1-carboxylate

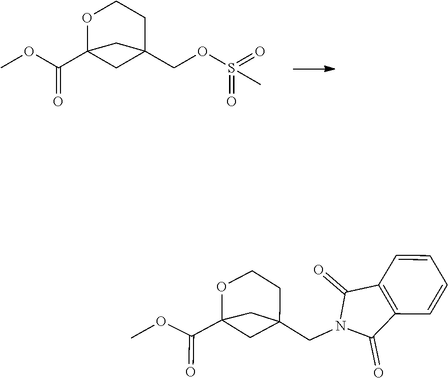

A suspension of methyl 5-(methylsulfonyloxymethyl)-2-oxabicyclo[3.1.1]heptane-1-carboxylate (100 mg, 0.303 mmol) and potassium phthalimide (62.0 mg, 0.333 mmol) in DMF (4 mL) was stirred at 100 °C for 3 h. The mixture was cooled to rt, taken up in water (20 mL) and washed with EtOAc (2×30 mL). The combined organic layers were washed with brine (10 mL), dried (Na₂SO₄) and concentrated to afford the product (98 mg, 82% yield).

[M+H]⁺=316.0

¹H NMR (CDCl₃, 400 MHz) δ 2.01-2.14 (4H, m), 2.30-2.39 (2H, m), 3.73 (5H, s), 4.15-4.21 (2H, m), 7.71-7.79 (2H, m), 7.83-7.90 (2H, m)

### [5-(Aminomethyl)-2-oxabicyclo[3.1.1]heptan-1-yl]methanol

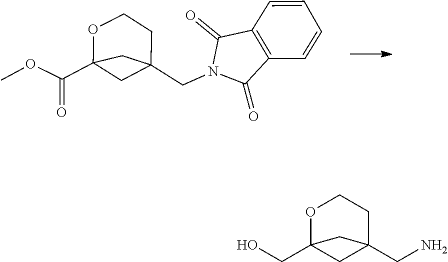 [SMILES: COC(=O)C12CC(CN3C(=O)c4ccccc4C3=O)(CCO1)C2 ; NCC12CCOC(CO)(C1)C2]

To a stirred solution of methyl 5-[(1,3-dioxoisoindol-2-yl)methyl]-2-oxabicyclo[3.1.1]heptane-1-carboxylate (98 mg, 0.25 mmol) in IPA (4 mL) and water (1 mL), was added NaBH₄ (56 mg, 1.49 mmol). After stirring for 24 h, AcOH (256 μL, 4.48 mmol) was added slowly and when the foaming subsided, the reaction was heated to 80 °C for 22 h. The reaction mixture was concentrated, the residue taken up in MeOH (1 mL) and the mixture was passed directly through an SCX column and washed with MeOH. The product was eluted with a solution of 1M NH₃ in MeOH and concentrated to afford the product (39 mg, 100% yield).

[M+H]⁺=158.1

¹H NMR (CDCl₃, 400 MHz) δ 1.60-1.67 (2H, m), 1.82 (2H, dd, J=7.2, 2.7 Hz), 1.92-1.98 (2H, m), 2.64 (2H, s), 3.51 (2H, s), 4.07-4.16 (2H, m)

### Intermediate 14

### tert-Butyl N-[[5-(hydroxymethyl)-2-oxabicyclo[3.1.1]heptan-1-yl]methyl]carbamate

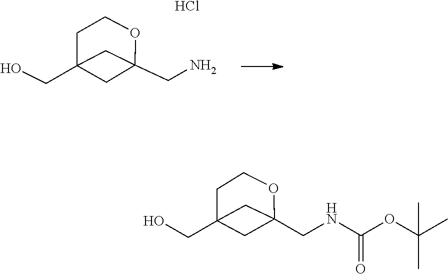 [SMILES: Cl ; NCC12CC(CO)(CCO1)C2 ; CC(C)(C)OC(=O)NCC12CC(CO)(CCO1)C2]

To a solution of [1-(aminomethyl)-2-oxabicyclo[3.1.1]heptan-5-yl]methanol hydrochloride (500 mg, 2.58 mmol) and TEA (1260 μL, 9.04 mmol) in THF (10 mL) at 0 °C was added Boc₂O (676 mg, 3.1 mmol) and the mixture was stirred for 10 min at 0 °C. After that time the reaction was stirred for 18 h at rt. The reaction was quenched with water (30 mL), washed with EtOAc (2×50 mL), dried (MgSO₄) and concentrated to afford the product (760 mg, 92% yield).

[M+Na]⁺=280.2

¹H NMR (CDCl₃) δ 1.43 (9H, s), 1.74-1.79 (4H, m), 1.94 (2H, t, J=6.9 Hz), 2.53 (1H, d, J=4.4 Hz), 3.17 (2H, d, J=5.8 Hz), 3.46 (2H, d, J=5.5 Hz), 4.07 (2H, t, J=6.9 Hz), 4.94 (1H, s)

### Intermediate 15

### [1-[[(2-Methylpropan-2-yl)oxycarbonylamino]methyl]-2-oxabicyclo[3.1.1]heptan-5-yl]methyl methanesulfonate

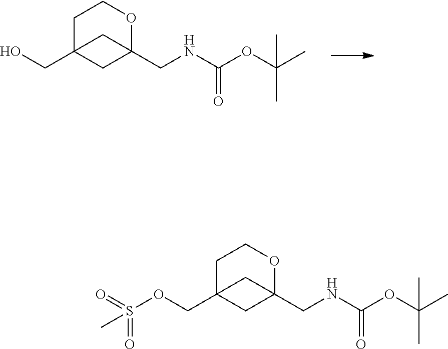 [SMILES: CC(C)(C)OC(=O)NCC12CC(CO)(CCO1)C2 ; CC(C)(C)OC(=O)NCC12CC(COS(C)(=O)=O)(CCO1)C2]

According to a modification of General Method 1a, tert-butyl N-[[5-(hydroxymethyl)-2-oxabicyclo[3.1.1]heptan-1-yl]methyl]carbamate (760 mg, 2.36 mmol) was reacted with MsCl (219 μL, 2.84 mmol) at 0 °C for 90 min. Aqueous work up afforded the product (950 mg, 96% yield) which was used without further purification.

[M+Na]⁺=358.3

¹H NMR (CDCl₃) δ 1.44 (9H, s), 1.81-1.89 (4H, m), 2.00 (2H, t, J=6.8 Hz), 3.02 (3H, s), 3.18 (2H, d, J=6.0 Hz), 4.06-4.10 (4H, m), 4.90 (1H, s)

### Intermediate 16

### 2-(tert-Butyl) 1-methyl 4-(hydroxymethyl)-2-azabicyclo[2.1.1]hexane-1,2-dicarboxylate

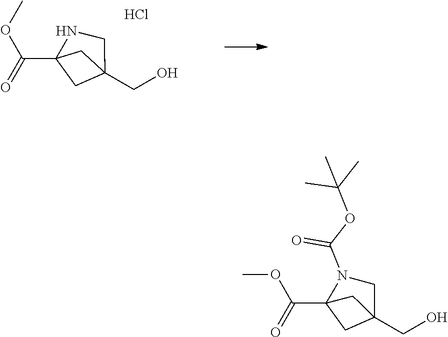 [SMILES: COC(=O)C12CC(CO)(CN1)C2 ; Cl ; COC(=O)C12CC(CO)(CN1C(=O)OC(C)(C)C)C2]

To a solution of methyl 4-(hydroxymethyl)-2-azabicyclo[2.1.1]hexane-1-carboxylate; hydrochloride (40.5 g, 176 mmol) and TEA (75 mL, 538 mmol) in anhydrous DCM (200 mL) and anhydrous MeOH (200 mL) at rt was added Boc₂O (42.1 g, 193 mmol). After stirring for 16 h. The reaction was quenched with brine (200 mL). The layers were separated, and the aqueous layer was extracted with DCM (2×100 mL). The combined organic phases were dried (Na₂SO₄), filtered, and concentrated. Purification by flash chromatography (Silica, 0-20% MeOH in DCM) provided the product (37.1 g, 74% yield).

[M+H]⁺=272.1

¹H NMR (CDCl₃, 400 MHz) δ 1.41 (9H, s), 1.75 (2H, dd, J=4.7, 1.9 Hz), 1.95-2.06 (2H, m), 2.17 (1H, s), 3.37 (2H, s), 3.76 (3H, s), 3.79 (2H, s)

### Intermediate 17

### 2-O-Benzyl 1-O-methyl 4-(methylsulfonyloxymethyl)-2-azabicyclo[2.1.1]hexane-1,2-dicarboxylate

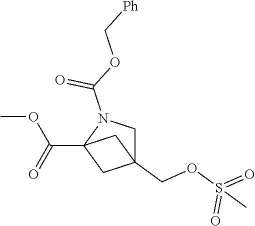 [SMILES: COC(=O)C12CC(COS(C)(=O)=O)(CN1C(=O)OCc1ccccc1)C2]

### 2-O-Benzyl 1-O-methyl 4-(hydroxymethyl-2-azabicyclo[2.1.1]hexane-1,2-dicarboxylate

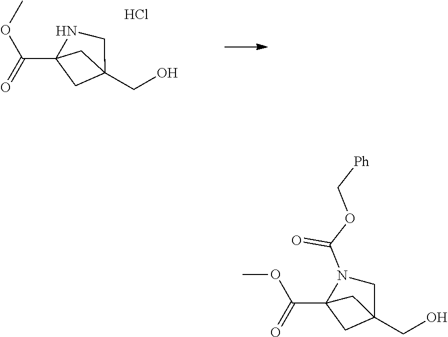 [SMILES: COC(=O)C12CC(CO)(CN1)C2 ; Cl ; COC(=O)C12CC(CO)(CN1C(=O)OCc1ccccc1)C2]

Methyl 4-(hydroxymethyl)-2-azabicyclo[2.1.1]hexane-1-carboxylate hydrochloride (600 mg, 2.89 mmol) was dissolved in DCM (15 mL) and cooled to 0 °C TEA (805 μL, 5.78 mmol) was added followed by benzyl chloroformate (407 μL, 2.89 mmol). The reaction was stirred at rt overnight before retreating with benzyl chloroformate (200 μL, 1.42 mmol) at 0 °C and stirring at rt for an additional 1 h. DCM (30 mL) and water (10 mL) were added. The organic layer was dried (MgSO₄), filtered and concentrated. The crude product was purified by flash chromatography (Silica, 20-100% EtOAc in Pet. Ether) to afford the product (465 mg, 53% yield).

[M+H]⁺=306.2

¹H NMR (DMSO-d6, 400 MHz) δ 1.56-1.66 (2H, m), 1.89-1.97 (2H, m), 3.32 (2H, s), 3.50-3.65 (5H, m), 4.74-4.78 (1H, m), 5.04 (2H, s), 7.30-7.42 (5H, m)

### 2-O-Benzyl 1-O-methyl 4-methylsulfonyloxymethyl)-2-azabicyclo[2.1.1]hexane-12-dicarboxylate

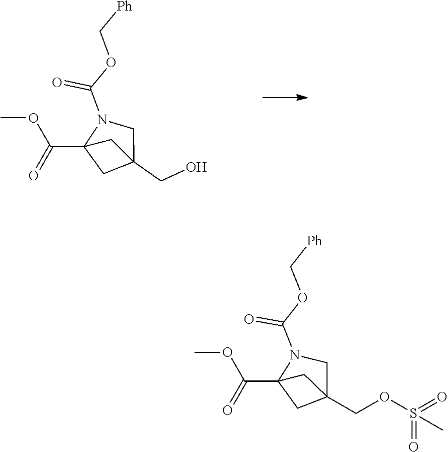

Following General Method 1a, 2-O-benzyl 1-O-methyl 4-(hydroxymethyl)-2-azabicyclo[2.1.1]hexane-1,2-dicarboxylate (1206 mg, 3.95 mmol) was reacted with MsCl (367 μL, 4.74 mmol) for 90 min, after which time aqueous work up afforded the product (1480 mg, 98% yield).

[M+H]⁺=384.1.

¹H NMR (CDCl₃, 400 MHz) δ 1.91 (2H, dd, J=4.8, 1.9 Hz), 2.12 (2H, dd, J=5.0, 1.9 Hz), 3.04 (3H, s), 3.51 (2H, s), 3.67 (3H, s), 4.40 (2H, s), 5.11 (2H, s), 7.27-7.44 (5H, m).

### Intermediate 18

### 2-(tert-Butyl) 1-methyl 4-(((tert-butyldiphenylsilyl)oxy)methyl)-2-azabicyclo[2.1.1]hexane-1,2-dicarboxylate

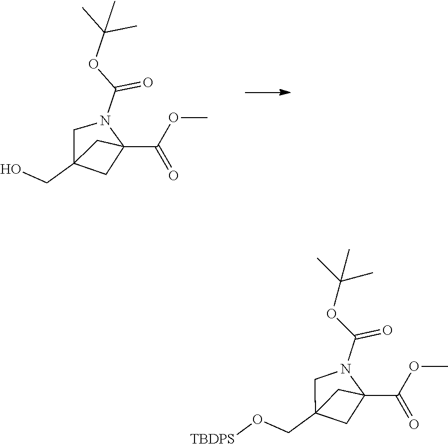

To a solution of 2-(tert-butyl) 1-methyl 4-(hydroxymethyl)-2-azabicyclo[2.1.1]hexane-1,2-dicarboxylate (30 g, 111 mmol) and imidazole (8.28 g, 122 mmol) in anhydrous DCM (150 mL) at 0 °C was added TBDPS-Cl (31.6 mL, 122 mmol) and the reaction stirred for 1 h at 0 °C, then at rt and for 16 h. After quenching with brine (200 mL) and EtOAc (100 mL), the layers were separated, and the aqueous layer was extracted with EtOAc (2×100 mL). The combined organic layers were washed with 1M HCl (aq) (200 mL), brine (200 mL), 1M NaOH (aq) (200 mL), dried (Na₂SO₄), filtered, and concentrated to afford the product (59.5 g, 95% yield).

¹H NMR (CDCl₃, 300 MHz) δ 1.06 (9H, s), 1.44 (9H, s), 1.75 (2H, d, J=4.6 Hz), 2.01 (2H, d, J=4.7 Hz), 3.37 (2H, s), 3.78-3.79 (3H, m), 3.80 (2H, s), 7.35-7.46 (6H, m), 7.58-7.67 (4H, m)

### Intermediate 19

### tert-Butyl 4-(((1,6-dimethyl-2-oxo-1,2-dihydropyridin-4-yl)oxy)methyl)-1-(hydroxymethyl)-2-azabicyclo[2.1.1]hexane-2-carboxylate

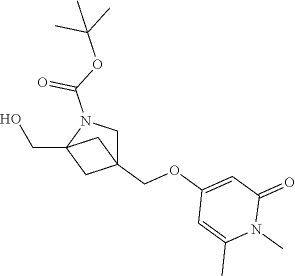 [SMILES: Cc1cc(OCC23CN(C(=O)OC(C)(C)C)C(CO)(C2)C3)cc(=O)n1C]

### 2-(tert-Butyl) 1-methyl 4-(((1,6-dimethyl-2-oxo-1,2-dihydropyridin-4-yl)oxy)methyl)-2-azabicyclo[2.1.1]hexane-1,2-dicarboxylate

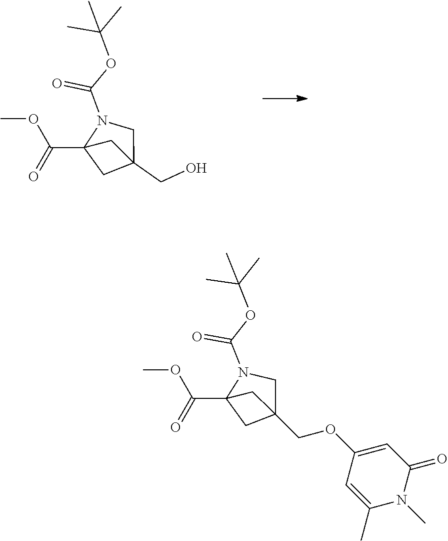 [SMILES: COC(=O)C12CC(CO)(CN1C(=O)OC(C)(C)C)C2 ; COC(=O)C12CC(COc3cc(C)n(C)c(=O)c3)(CN1C(=O)OC(C)(C)C)C2]

According to General Method 15, 2-(tert-butyl) 1-methyl 4-(hydroxymethyl)-2-azabicyclo[2.1.1]hexane-1,2-dicarboxylate (10.0 g, 36.8 mmol) was reacted with 4-hydroxy-1,6-dimethyl-pyridin-2-one (6.00 g, 41.0 mmol). The reaction mixture was filtered over Celite®. The solid was washed with EtOAc (200 mL) and the filtrate was concentrated. Purification by flash chromatography (silica, 0-7% MeOH in EtOAc) afforded the product (6.20 g, 30% yield, 70% pure).

[M+H]⁺=393.2

¹H NMR (DMSO-d6, 400 MHz) δ 1.36 (9H, s), 1.67 (2H, dd, J=4.5, 1.9 Hz), 2.01-2.09 (2H, m), 3.32 (3H, s), 3.35 (2H, s), 3.66 (3H, s), 3.69 (3H, s), 4.16 (2H, s), 5.69 (1H, d, J=2.8 Hz), 5.89 (1H, dd, J=2.8, 0.9 Hz)

### tert-Butyl 4-(((1,6-dimethyl-2-oxo-1,2-dihydropyridin-4-yl)oxy)methyl)-1-(hydroxymethyl)-2-azabicyclo[2.1.1]hexane-2-carboxylate

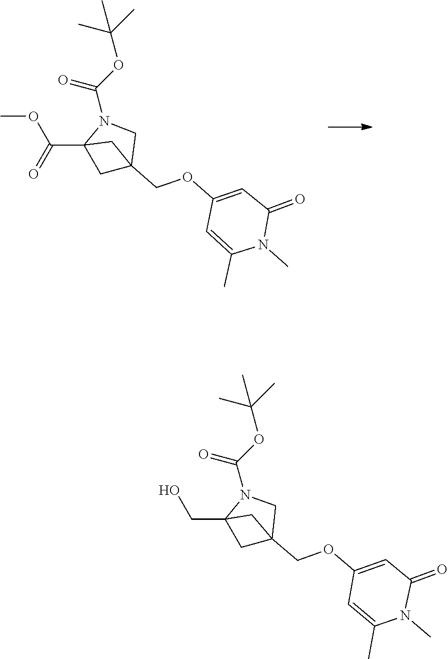 [SMILES: COC(=O)C12CC(COc3cc(C)n(C)c(=O)c3)(CN1C(=O)OC(C)(C)C)C2 ; Cc1cc(OCC23CN(C(=O)OC(C)(C)C)C(CO)(C2)C3)cc(=O)n1C]

According to a variation of General Method 3b, 2-(tert-butyl) 1-methyl 4-(((1,6-dimethyl-2-oxo-1,2-dihydropyridin-4-yl)oxy)methyl)-2-azabicyclo[2.1.1]hexane-1,2-dicarboxylate (6.20 g, 11.1 mmol) was reduced in the presence of LiBH₄ (2M in THF, 11 mL, 22.0 mmol) for 72 h. The mixture was quenched with H₂O (30 mL) and concentrated. The aqueous phase was extracted with DCM (3×60 mL). The combined organic layers were dried (Na₂SO₄), filtered, and concentrated. Purification by flash chromatography (Silica, 0-12% MeOH in EtOAc) afforded the product (2.70 g, 67% yield).

[M+H]⁺=366.1

¹H NMR (CDCl₃, 400 MHz) δ 1.48 (9H, s), 1.66 (1H, s), 1.70-1.79 (4H, m), 2.30 (3H, d, J=0.7 Hz), 3.37 (2H, s), 3.45 (3H, s), 3.94 (2H, d, J=6.4 Hz), 4.04 (2H, s), 5.79 (2H, d, J=0.8 Hz)

### Intermediate 20

### (4-(((1-((2,4-Dimethoxybenzyl)amino)isoquinolin-5-yl)amino)methyl)-2-oxabicyclo[2.1.1]hexan-1-yl)methanol

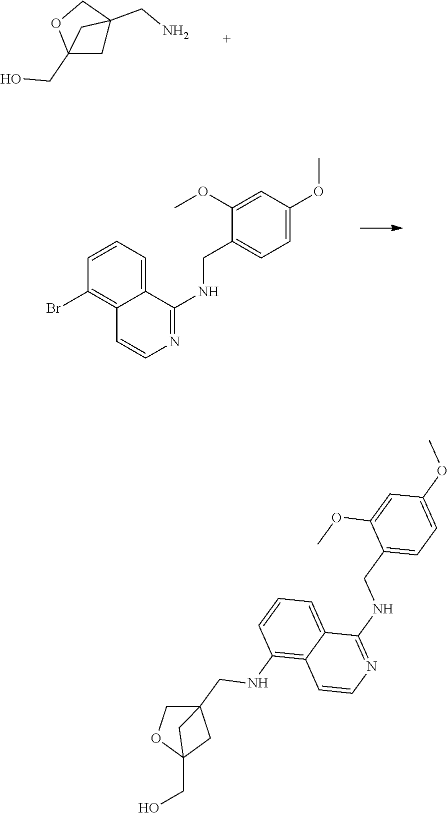 [SMILES: NCC12COC(CO)(C1)C2 ; COc1ccc(CNc2nccc3c(Br)cccc23)c(OC)c1 ; COc1ccc(CNc2nccc3c(NCC45COC(CO)(C4)C5)cccc23)c(OC)c1]

According General Method 4, a suspension of [4-(aminomethyl)-2-oxabicyclo[2.1.1]hexan-1-yl]methanol (500 mg, 3.49 mmol), 5-bromo-N-(2,4-dimethoxybenzyl)isoquinolin-1-amine (1434 mg, 3.84 mmol) and NaO^{t}Bu (503 mg, 5.24 mmol) in 1,4-dioxane (5 mL) was reacted in the presence of Brettphos Pd G4 (241 mg, 0.26 mmol) in a sealed vial at 40 °C for 18 h and then at 60 °C for 2 h. Flash chromatography (Silica, 20-100% EtOAc in Pet. Ether) afforded the product (861 mg, 57% yield).

[M+H]⁺=436.2

### Intermediate 21

### (4-(((1-((2,4-dimethoxybenzyl)amino)isoquinolin-5-yl)amino)methyl)-2-oxabicyclo[2.1.1]hexan-1-yl)methyl methanesulfonate

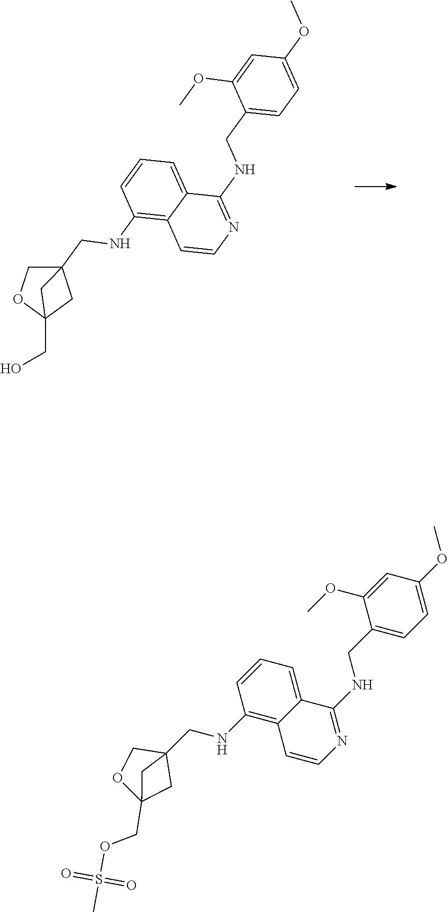 [SMILES: COc1ccc(CNc2nccc3c(NCC45COC(CO)(C4)C5)cccc23)c(OC)c1 ; COc1ccc(CNc2nccc3c(NCC45COC(COS(C)(=O)=O)(C4)C5)cccc23)c(OC)c1]

According to General Method 1a, a solution of (4-(((1-((2,4-dimethoxybenzyl)amino)isoquinolin-5-yl)amino)methyl)-2-oxabicyclo[2.1.1]hexan-1-yl)methanol (800 mg, 1.84 mmol) was reacted with methane sulfonyl chloride (252 mg, 2.2 mmol) to afford the product (914 mg, 97% yield) which was used without purification.

[M+H]⁺=514.2

### Intermediate 22

### (1-(((1-((2,4-Dimethoxybenzyl)amino)isoquinolin-5-yl)amino)methyl)-2-oxabicyclo[2.1.1]hexan-4-yl)methanol

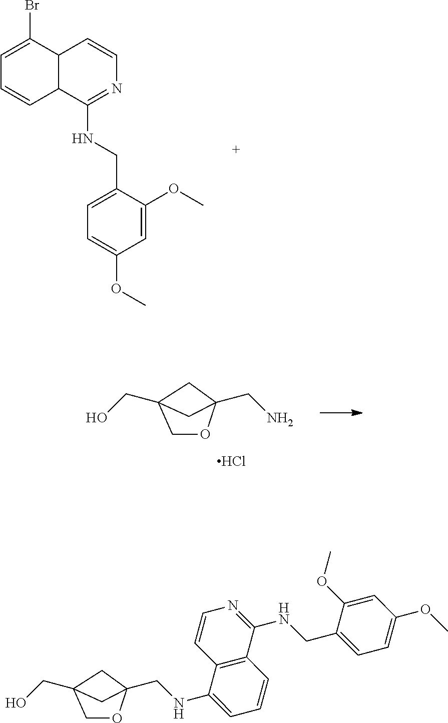 [SMILES: COc1ccc(CNC2=NC=CC3C(Br)=CC=CC23)c(OC)c1 ; NCC12CC(CO)(CO1)C2 ; Cl ; COc1ccc(CNc2nccc3c(NCC45CC(CO)(CO4)C5)cccc23)c(OC)c1]

According to General Method 4, a suspension of [1-(aminomethyl)-2-oxabicyclo[2.1.1]hexan-4-yl]methanol hydrochloride (250 mg, 1.39 mmol), 5-bromo-N-(2,4-dimethoxybenzyl)isoquinolin-1-amine (571 mg, 1.53 mmol) and NaO^{t}Bu (334 mg, 3.48 mmol) in 1,4-dioxane (5 mL) was reacted in the presence of Brettphos Pd G4 (96 mg, 0.1 mmol) in a sealed vial at 40 °C for 18 h. Flash chromatography (Silica, 20-100% EtOAc in Pet. Ether) afforded the product (429 mg, 71% yield).

[M+H]⁺=436.2

### Intermediate 23

### (1-(((1-((2,4-Dimethoxybenzyl)amino)isoquinolin-5-yl)amino)methyl)-2-oxabicyclo[2.1.1]hexan-4-yl)methyl methanesulfonate

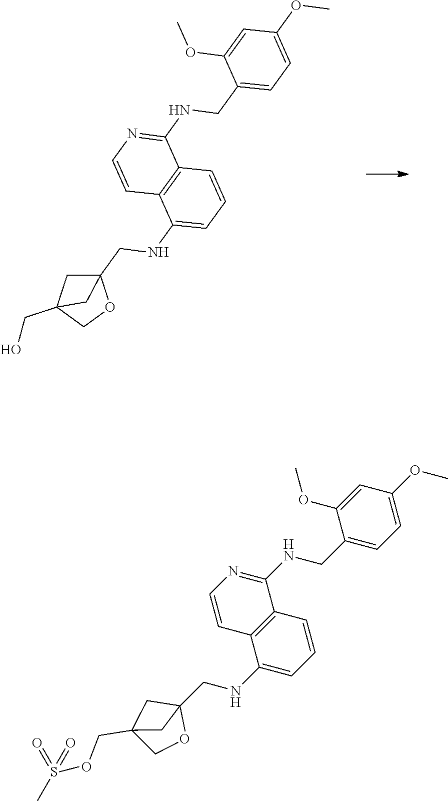 [SMILES: COc1ccc(CNc2nccc3c(NCC45CC(CO)(CO4)C5)cccc23)c(OC)c1 ; COc1ccc(CNc2nccc3c(NCC45CC(COS(C)(=O)=O)(CO4)C5)cccc23)c(OC)c1]

According to General Method 1a, a solution of (1-(((1-((2,4-dimethoxybenzyl)amino)isoquinolin-5-yl)amino)methyl)-2-oxabicyclo[2.1.1]hexan-4-yl)methanol (429 mg, 0.99 mmol) was reacted with methane sulfonyl chloride (135 mg, 1.18 mmol) to afford the product (440 mg, 87% yield) which was used without purification by chromatography.

[M+H]⁺=514.2

### Intermediate 24

### (1-(((1-((2,4-Dimethoxybenzyl)amino)isoquinolin-5-yl)amino)methyl)-2-oxabicyclo[3.1.1]heptan-5-yl)methanol

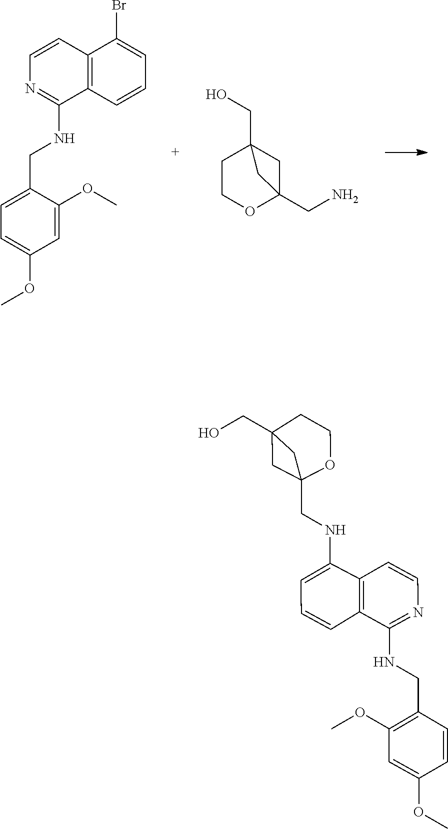 [SMILES: COc1ccc(CNc2nccc3c(Br)cccc23)c(OC)c1 ; NCC12CC(CO)(CCO1)C2 ; COc1ccc(CNc2nccc3c(NCC45CC(CO)(CCO4)C5)cccc23)c(OC)c1]

According to General Method 4, a suspension of [1-(aminomethyl)-2-oxabicyclo[3.1.1]heptan-5-yl]methanol hydrochloride (300 mg, 1.55 mmol), 5-bromo-N-(2,4-dimethoxybenzyl)isoquinolin-1-amine (578 mg, 1.55 mmol) and NaO^{t}Bu (372 mg, 3.87 mmol) in 1,4-dioxane (6 mL) was reacted in the presence of Brettphos Pd G4 (107 mg, 0.12 mmol) in a sealed vial at 40 °C for 3 days and at 60 °C for 18 h. Flash chromatography (Silica, 0-100% EtOAc in Pet. Ether followed by 0-12% MeOH in EtOAc) afforded the product (190 mg, 27% yield).

[M+H]⁺=450.3

### Intermediate 25

### (1-(((1-((2,4-Dimethoxybenzyl)amino)isoquinolin-5-yl)amino)methyl)-2-oxabicyclo[3.1.1]heptan-5-yl)methyl methanesulfonate

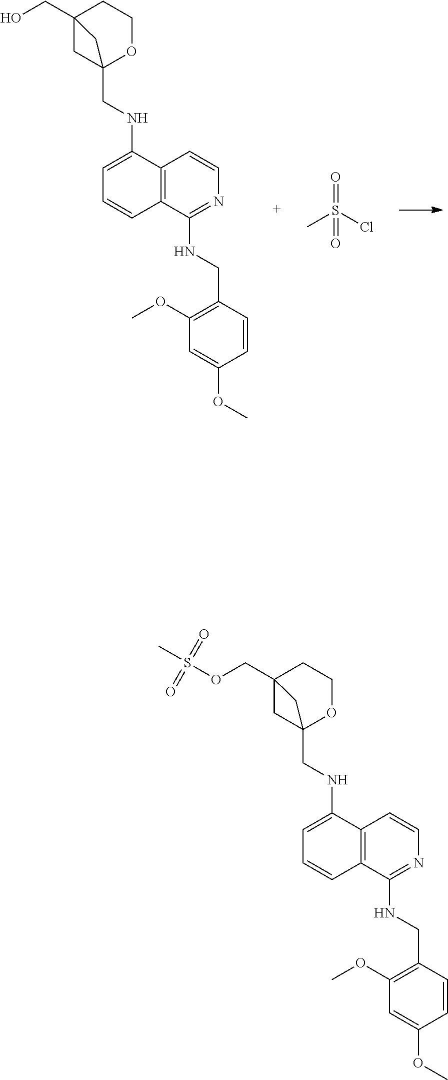

According to General Method 1a, a solution of (1-(((1-((2,4-dimethoxybenzyl)amino)isoquinolin-5-yl)amino)methyl)-2-oxabicyclo[3.1.1]heptan-5-yl)methanol (160 mg, 0.36 mmol) was reacted with MsCl (36 μL, 0.46 mmol) to afford the product (190 mg, 91% yield) which was used without purification.

[M+H]⁺=528.2

¹H NMR (CDCl₃) δ 1.45 (9H, s), 1.92 (4H, s), 2.07 (2H, t, J=6.9 Hz), 3.23 (2H, d, J=5.9 Hz), 3.74 (2H, s), 4.09-4.14 (2H, m), 5.03 (1H, t, J=5.9 Hz), 6.96 (1H, dd, J=9.7, 2.3 Hz), 7.48-7.51 (2H, m), 7.56 (1H, d, J=0.8 Hz), 7.66 (1H, d, J=1.8 Hz)

### Intermediate 26

### 4-((4-(((1-((2,4-Dimethoxybenzyl)amino)isoquinolin-5-yl)amino)methyl)-2-azabicyclo[2.1.1]hexan-1-yl)methoxy)-1-methylpyridin-2(1H)-one

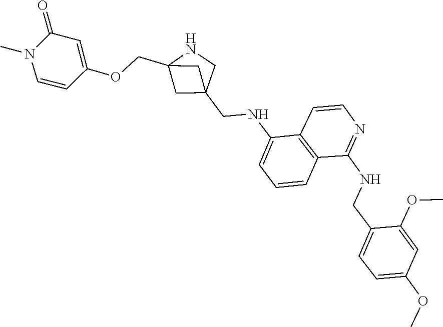

### 2-O-Benzyl 1-O-methyl 4-(azidomethyl)-2-azabicyclo[2.1.1]hexane-1,2-dicarboxylate

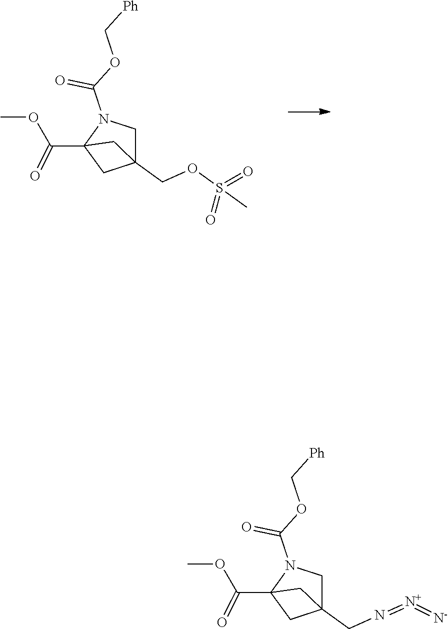 [SMILES: COC(=O)C12CC(COS(C)(=O)=O)(CN1C(=O)OCc1ccccc1)C2 ; COC(=O)C12CC(CN=[N+]=[N-])(CN1C(=O)OCc1ccccc1)C2]

To 2-O-benzyl 1-O-methyl 4-(methylsulfonyloxymethyl)-2-azabicyclo[2.1.1]hexane-1,2-dicarboxylate (458 mg, 1.19 mmol) in DMF (5 mL) was added NaN₃ (155 mg, 2.39 mmol) at rt. The mixture was stirred at rt for 4 days. The mixture was diluted with EtOAc (50 mL) and washed with water (4×10 mL) and brine (10 mL), dried (MgSO₄) and concentrated to afford the product (330 mg, 75% yield).

[M+H]⁺=331.1

¹H NMR (CD₃CN) δ 1.73 (2H, dd, J=4.7, 1.9 Hz), 2.01 (2H, dd, J=4.6, 1.4 Hz), 3.38 (2H, s), 3.56 (5H, s), 5.04 (21H, s), 7.28-7.37 (5H, m)

### 2-O-Benzyl 1-O-methyl 4-(azidomethyl)-2-azabicyclo[2.1.1]hexane-1,2-dicarboxylate

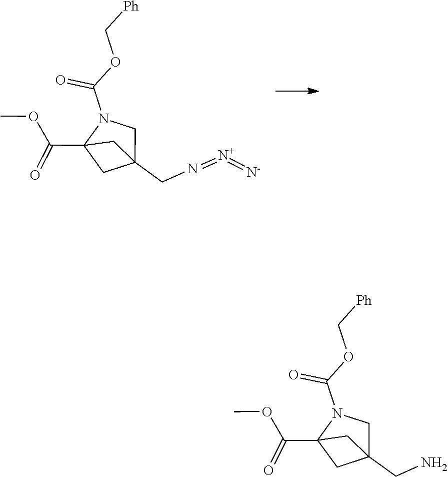 [SMILES: COC(=O)C12CC(CN=[N+]=[N-])(CN1C(=O)OCc1ccccc1)C2 ; COC(=O)C12CC(CN)(CN1C(=O)OCc1ccccc1)C2]

2-O-Benzyl 1-O-methyl 4-(azidomethyl)-2-azabicyclo[2.1.1]hexane-1,2-dicarboxylate (330 mg, 0.9 mmol) was dissolved in THF (6 mL). Triphenylphosphine, polymer bound (3 mmol/1 g of resin) (453 mg, 1.35 mmol) was added and the reaction stirred at rt for 2 h. Water (162 μL, 8.99 mmol) was added and the reaction heated at reflux for 3 h. The mixture was cooled to rt and filtered, washing the resin with 10% MecOH in DCM (25 mL). The filtrate was concentrated to afford the product (240 mg, 79% yield).

[M+H]⁺=305.1

¹H NMR (CDCl₃) δ 1.29 (2H, br s), 1.70 (2H, dd, J=4.6, 1.7 Hz), 1.90 (2H, d, J=4.6 Hz), 2.85 (2H, s), 3.34 (2H, s), 3.53 (3H, br s), 5.02 (2H, s), 7.19-7.28 (5H, m)

### 2-Benzyl 1-methyl 4-(((1-((2,4-dimethoxybenzyl)amino)isoquinolin-5-yl)amino)methyl)-2-azabicyclo[2.1.1]hexane-1,2-dicarboxylate

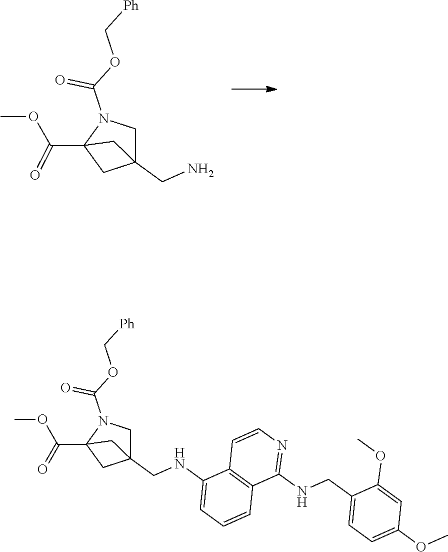

According to a variation of General Method 4, 5-bromo-N-(2,4-dimethoxybenzyl)isoquinolin-1-amine (2239 mg, 6.00 mmol) was reacted with 2-O-benzyl 1-O-methyl 4-(aminomethyl)-2-azabicyclo[2.1.1]hexane-1,2-dicarboxylate (1927 mg, 5.70 mmol), Cs₂CO₃ (5610 mg, 17.0 mmol) and Brettphos Pd G4 (801 mg, 0.852 mmol) in 1,4-dioxane (25 mL) at 65 °C for 6 h. The reaction mixture was cooled, filtered (Celite®) washing with EtOAc (200 mL), and the filtrate was concentrated. Purification by flash chromatography (Silica, 0-70% EtOAc in hexanes) afforded the product (2580 mg, 76%).

[M+H]⁺=597.3

¹H NMR (CDCl₃, 500 MHz) δ 1.91 (2H, dd, J=4.7, 1.9 Hz), 2.11 (2H, d, J=4.8 Hz), 3.51-3.70 (7H, m), 3.80 (3H, s), 3.86 (3H, s), 4.26 (1H, s), 4.72 (2H, d, J=5.3 Hz), 5.12 (2H, s), 5.63 (1H, t, J=5.3 Hz), 6.45 (1H, dd, J=8.3, 2.4 Hz), 6.50 (1H, d, J=2.4 Hz), 6.66 (1H, d, J=7.7 Hz), 6.75 (1H, d, J=6.1 Hz), 7.08 (1H, d, J=8.3 Hz), 7.28-7.39 (7H, m), 8.02 (1H, d, J=6.1 Hz)

### Benzyl 4-(((1-((2,4-dimethoxybenzyl)amino)isoquinolin-5-yl)amino)methyl)-1-(hydroxymethyl)-2-azabicyclo[2.1.1]hexane-2-carboxylate

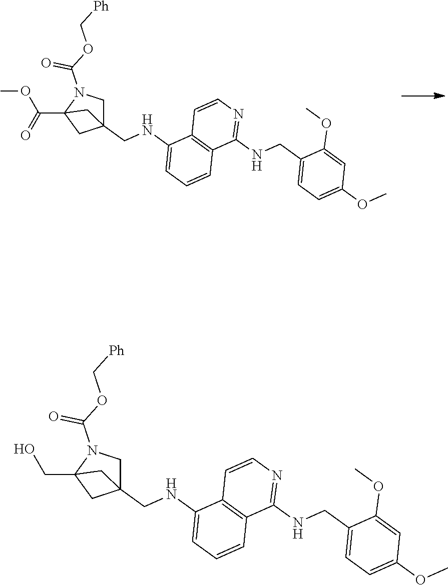

Following a variation of General Method 3b, 2-benzyl 1-methyl 4-(((1-((2,4-dimethoxybenzyl)amino)isoquinolin-5-yl)amino)methyl)-2-azabicyclo[2.1.1]hexane-1,2-dicarboxylate (1713 mg, 2.87 mmol) was reduced in the presence of lithium triethylborohydride (1M in THF, 7.2 mL, 7.20 mmol) at 0 °C for 2.5 h. The reaction mixture was diluted with water (10 mL), brine (20 mL) and EtOAc (90 mL). The phases were separated, and the aqueous phase was extracted with EtOAc (2×50 mL). The combined organic phases were dried (MgSO₄) and concentrated. Purification by flash chromatography (Silica, 20-68% EtOAc in hexanes) afforded the product (1096 mg, 67% yield).

[M+H]⁺=569.8

¹H NMR (CDCl₃, 400 MHz) δ 1.78 (4H, s), 3.51 (2H, s), 3.54 (2H, s), 3.80 (3H, s), 3.86 (3H, s), 3.98 (2H, d, J=7.4 Hz), 4.24 (1H, s), 4.54 (1H, s), 4.72 (2H, d, J=5.3 Hz), 5.14 (2H, s), 5.63 (1H, t, J=5.4 Hz), 6.45 (1H, dd, J=8.2, 2.4 Hz), 6.50 (1H, d, J=2.4 Hz), 6.67 (1H, dd, J=7.8, 0.8 Hz), 6.76 (1H, dd, J=6.1, 0.9 Hz), 7.05-7.11 (1H, m), 7.37 (7H, d, J=4.6 Hz), 8.02 (1H, d, J=6.1 Hz)

### Benzyl 4-(((1-((2,4-dimethoxybenzyl)amino)isoquinolin-5-yl)amino)methyl)-1-(((1-methyl-2-oxo-1,2-dihydropyridin-4-yl)oxy)methyl)-2-azabicyclo[2.1.1]hexane-2-carboxylate

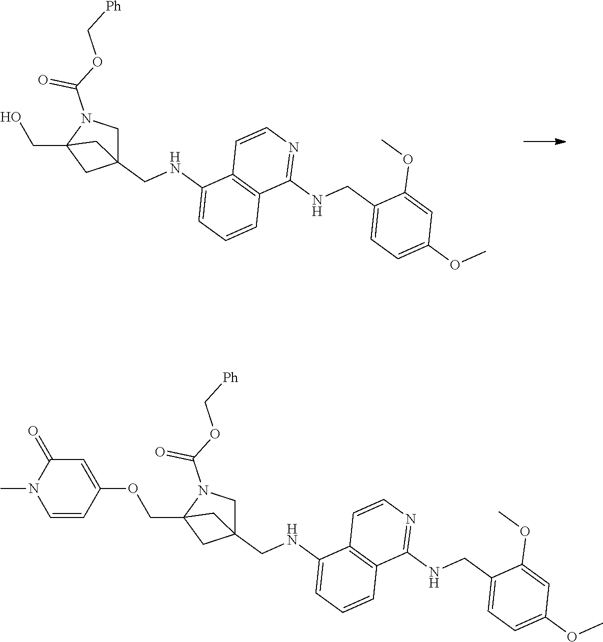

According to General Method 15, benzyl 4-(((1-((2,4-dimethoxybenzyl)amino)isoquinolin-5-yl)amino)methyl)-1-(hydroxymethyl)-2-azabicyclo[2.1.1]hexane-2-carboxylate (1046 mg, 1.84 mmol), was reacted with 4-hydroxy-1-methyl-pyridin-2-one (254 mg, 1.93 mmol). After aqueous work up, purification by flash column chromatography (Silica, 0-20% MeOH in EtOAc) afforded the product (319 mg, 26% yield).

[M+H]⁺=676.4

¹H NMR (CDCl₃, 400 MHz) δ 1.73 (2H, dd, J=4.5, 1.9 Hz), 2.01-2.04 (2H, m), 3.46 (3H, s), 3.55 (2H, s), 3.57 (3H, s), 3.80 (3H, s), 3.86 (3H, s), 4.26 (1H, s), 4.47-4.56 (2H, m), 4.73 (2H, d, J=5.3 Hz), 5.10 (2H, s), 5.62 (1H, d, J=5.5 Hz), 5.82-5.94 (2H, m), 6.45 (1H, dd, J=8.2, 2.4 Hz), 6.50 (1H, d, J=2.4 Hz), 6.68 (1H, d, J=7.7 Hz), 6.77 (1H, dd, J=6.2, 0.9 Hz), 7.08 (2H, dd, J=8.0, 4.4 Hz), 7.28-7.39 (6H, m), 8.03 (1H, d, J=6.1 Hz)

### 4-((4-(((1-((2,4-Dimethoxybenzyl)amino)isoquinolin-5-yl)amino)methyl)-2-azabicyclo[2.1.1]hexan-1-yl)methoxy)-1-methylpyridin-2(1H)-one

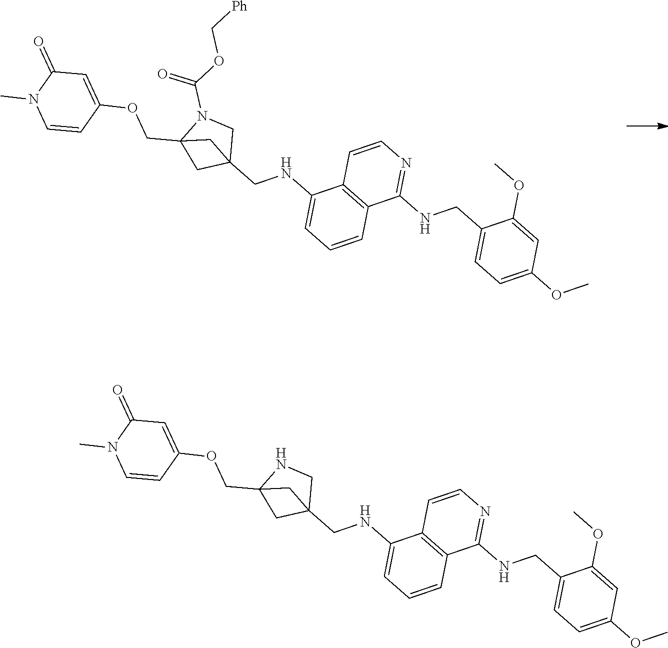

According to a variation of General Method 3e, a solution of benzyl 4-(((1-((2,4-dimethoxybenzyl)amino)isoquinolin-5-yl)amino)methyl)-1-(((1-methyl-2-oxo-1,2-dihydropyridin-4-yl)oxy)methyl)-2-azabicyclo[2.1.1]hexane-2-carboxylate (319 mg, 0.472 mmol) in MeOH (3 mL) was added to a mixture of Pd(OH)₂/C (20 wt %, 82.0 mg, 0.0117 mmol) in MeOH (1.8 mL). The mixture was subjected to hydrogenation at 1 atmosphere at rt for 4 h. The mixture was filtered over Celite® washing with MeOH (2×25 mL), and the filtrate was concentrated to afford the product (234 mg, 91% yield).

[M+H]⁺=542.4

1H NMR (DMSO-d6, 500 MHz) δ 1.34 (2H, dd, J=4.0, 1.7 Hz), 1.62 (2H, dd, J=4.2, 1.6 Hz), 2.83 (2H, s), 3.17 (1H, d, J=2.6 Hz), 3.31 (3H+H2O, s), 3.49 (2H, d, J=5.8 Hz), 3.71 (3H, s), 3.82 (3H, s), 4.03 (2H, s), 4.58 (2H, d, J=5.6 Hz), 5.76 (1H, d, J=2.8 Hz), 5.89 (1H, dd, J=7.5, 2.8 Hz), 5.95 (1H, t, J=6.0 Hz), 6.39 (1H, dd, J=8.4, 2.4 Hz), 6.55 (1H, d, J=2.4 Hz), 6.66 (1H, d, J=7.7 Hz), 7.02 (1H, d, J=8.4 Hz), 7.12 (1H, d, J=6.2 Hz), 7.23 (1H, t, J=8.0 Hz), 7.36 (1H, t, J=5.8 Hz), 7.42 (1H, d, J=8.4 Hz), 7.52 (1H, d, J=7.6 Hz), 7.72 (1H, d, J=6.0 Hz)

### Intermediate 27

### 4-((1-(((1-((2,4-Dimethoxybenzyl)amino)isoquinolin-5-yl)amino)methyl)-2-azabicyclo[2.1.1]hexan-4-yl)methoxy)-1-methylpyridin-2(1H)-one

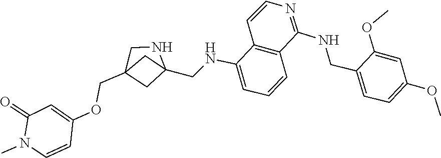

### 2-O-Benzyl 1-O-methyl 4-[(1-methyl-2-oxopyridin-4-yl)oxymethyl]-2-azabicyclo[2.1.1]hexane-1,2-dicarboxylate

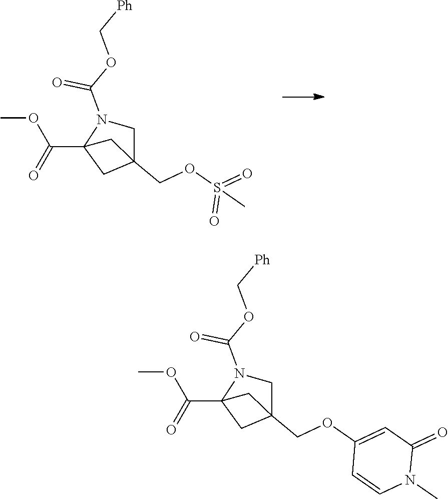 [SMILES: COC(=O)C12CC(COS(C)(=O)=O)(CN1C(=O)OCc1ccccc1)C2 ; COC(=O)C12CC(COc3ccn(C)c(=O)c3)(CN1C(=O)OCc1ccccc1)C2]

According to General Method 2b, 2-O-benzyl 1-O-methyl 4-(methylsulfonyloxymethyl)-2-azabicyclo[2.1.1]hexane-1,2-dicarboxylate (703 mg, 1.56 mmol) was reacted with 4-hydroxy-1-methyl-2-pyridone (195 mg, 1.56 mmol) and K₂CO₃ (646 mg, 4.68 mmol) in DMF (10 mL) at 90 °C for 17 h. After aqueous work up the residue was purified by flash chromatography (Silica, 0-9% MecOH in DCM) to afford the product (620 mg, 96% yield).

[M+H]⁺=413.4

¹H NMR (DMSO-d6, 400 MHz) δ 1.74 (2H, dd, J=4.6, 1.9 Hz), 2.10 (2H, dd, J=4.7, 1.8 Hz), 3.32 (3H, s), 3.45 (2H, s), 3.57 (3H, s), 4.19 (2H, s), 5.06 (2H, s), 5.79 (1H, d, J=2.8 Hz), 5.93 (1H, dd, J=7.6, 2.8 Hz), 7.36 (5H, m), 7.56 (1H, d, J=7.5 Hz)

### Benzyl 1-(hydroxymethyl)-4-[(1-methyl-2-oxopyridin-4-yl)oxymethyl]-2-azabicyclo[2.1.1]hexane-2-carboxylate

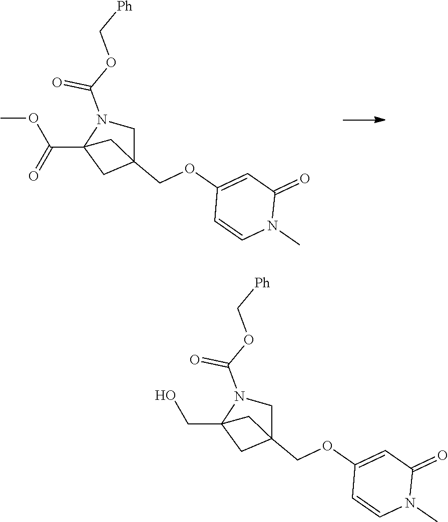

According to a modification of General Method 3b, 2-O-Benzyl 1-O-methyl 4-[(1-methyl-2-oxopyridin-4-yl)oxymethyl]-2-azabicyclo[2.1.1]hexane-1,2-dicarboxylate (582 mg, 1.41 mmol) was reduced in the presence of lithium borohydride (LiBH₄) (2M solution in THF) (776 μL, 1.55 mmol) over 72 h. Water (10 mL) and DCM (30 mL) were added. The organic layer was isolated and the aqueous layer re-extracted with DCM (3×10 mL). The combined organic layers were dried (MgSO₄), filtered and concentrated to afford the product (464 mg, 86% yield).

[M+H]=385.3

¹H NMR (DMSO-d6, 400 MHz) δ 1.46 (2H, dd, J=4.4, 1.8 Hz), 1.83-1.95 (2H, m), 3.33 (3H, s), 3.41 (2H, s), 3.90 (2H, d, J=6.1 Hz), 4.17 (2H, s), 4.70 (1H, t, J=6.2 Hz), 5.06 (2H, s), 5.80 (1H, d, J=2.8 Hz), 5.93 (1H, dd, J=7.5, 2.8 Hz), 7.27-7.36 (1H, m), 7.37 (4H, d, J=3.7 Hz), 7.56 (1H, d, J=7.6 Hz)

### Benzyl-1-formyl-4-[(1-methyl-2-oxopyridin-4-yl)oxymethyl]-2-azabicyclo[2.1.1]hexane-2-carboxylate

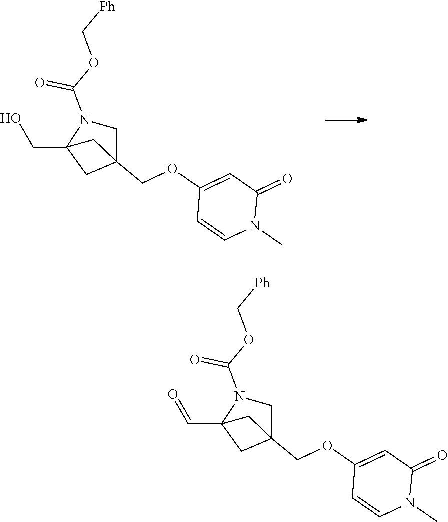

According to General Method 16 benzyl 1-(hydroxymethyl)-4-[(1-methyl-2-oxo-4-pyridyl)oxymethyl]-2-azabicyclo[2.1.1]hexane-2-carboxylate (1.20 g, 3.12 mmol) was oxidised. Aqueous work up afforded the product (2.00 g, 99% yield).

[M+H]⁺=384.4

¹H NMR (DMSO-d6, 400 MHz) δ 1.73 (2H, dd, J=4.4, 1.8 Hz), 2.05 (2H, d, J=5.7 Hz), 3.32 (3H, s), 3.50 (2H, s), 4.20 (2H, s), 5.12 (2H, s), 5.80 (1H, d, J=2.7 Hz), 5.94 (1H, dd, J=7.5, 2.8 Hz), 7.27-7.44 (5H, m), 7.56 (1H, d, J=7.6 Hz), 9.77 (1H, s)

### Benzyl 1-(((1-((2,4-dimethoxybenzyl)amino)isoquinolin-5-yl)amino)methyl)-4-(((1-methyl-2-oxo-1,2-dihydropyridin-4-yl)oxy)methyl)-2-azabicyclo[2.1.1]hexane-2-carboxylate

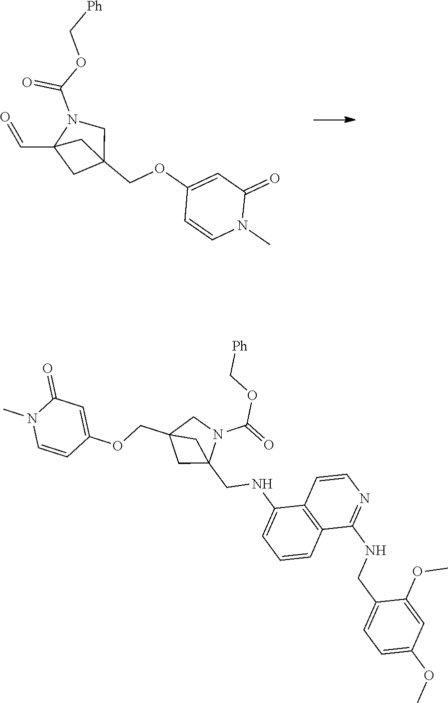

Following General Method 17, benzyl 1-formyl-4-[(1-methyl-2-oxo-4-pyridyl)oxymethyl]-2-azabicyclo[2.1.1]hexane-2-carboxylate (2.00 g, 60% purity, 3.14 mmol) was reacted with N1-(2,4-dimethoxybenzyl)isoquinoline-1,5-diamine (0.97 g, 3.14 mmol) for 24 h. After aqueous work up, the crude product was purified by flash chromatography (Silica, 0-30% MeOH in EtOAc) to afford the product (1.60 g, 67% yield).

[M+H]⁺=676.8

¹H NMR (DMSO-d6, 400 MHz) δ 1.55 (2H, d, J=3.1 Hz), 1.91 (2H, s), 3.31 (3H, s), 3.42 (2H, d, J=6.9 Hz), 3.71 (3H, s), 3.83 (3H, s), 3.95 (2H, d, J=6.1 Hz), 4.13 (2H, s), 4.59 (2H, d, J=5.6 Hz), 5.12 (2H, s), 5.76 (1H, d, J=2.7 Hz), 5.88 (1H, dd, J=7.5, 2.8 Hz), 6.01 (1H, s), 6.39 (1H, dd, J=8.4, 2.4 Hz), 6.55 (1H, d, J=2.4 Hz), 6.73 (1H, d, J=7.7 Hz), 6.88 (1H, d, J=6.1 Hz), 7.02 (1H, d, J=8.4 Hz), 7.24 (1H, t, J=8.0 Hz), 7.29-7.42 (6H, m), 7.46 (1H, d, J=8.4 Hz), 7.51 (1H, d, J=7.6 Hz), 7.71 (1H, d, J=6.1 Hz)

### 4-((1-(((1-((2,4-Dimethoxybenzyl)amino)isoquinolin-5-yl)amino)methyl)-2-azabicyclo[2.1.1]hexan-4-yl)methoxy)-1-methylpyridin-2(1H)-one

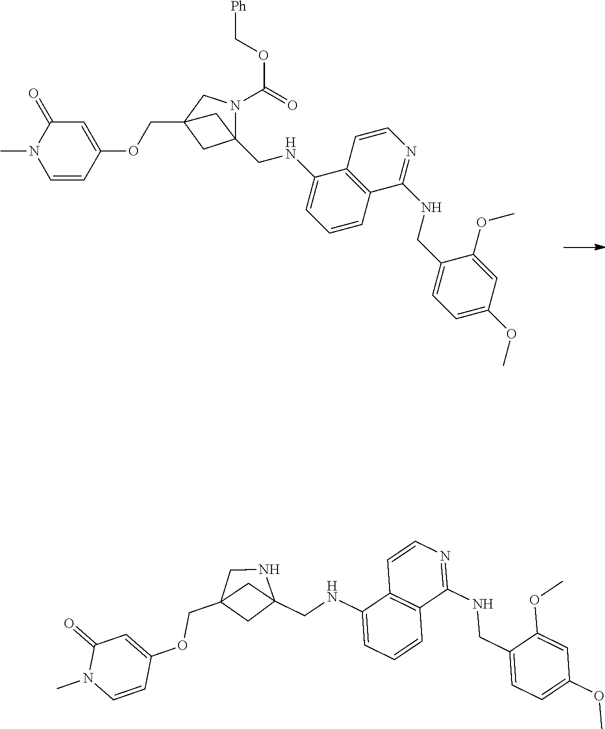

Following a variation of General Method 3e, a solution of benzyl 1-(((1-((2,4-dimethoxybenzyl)amino)isoquinolin-5-yl)amino)methyl)-4-(((1-methyl-2-oxo-1,2-dihydropyridin-4-yl)oxy)methyl)-2-azabicyclo[2.1.1]hexane-2-carboxylate (1350 mg, 2.00 mmol) in MeOH (10 mL) was added to a mixture of Pd(OH)₂/C (20 wt %) (338 mg, 0.481 mmol) in MeOH (10 mL). The mixture was subjected to hydrogenation at 1 atmosphere at rt for 66 h. The mixture was filtered over Celite® washing with MeOH (3×20 mL) and the filtrate was concentrated to afford the product (1100 mg, 95% yield).

[M+H]⁺=542.4

¹H NMR (DMSO-d6, 400 MHz) δ 1.41 (2H, s), 1.65 (2H, s), 2.87 (2H, s), 3.35 (3H, s), 3.44 (2H, d, J=5.1 Hz), 3.71 (3H, s), 3.82 (3H, s), 4.14 (2H, s), 4.59 (2H, d, J=5.4 Hz), 5.77 (1H, d, J=2.7 Hz), 5.83-5.94 (2H, m), 6.39 (1H, dd, J=8.4, 2.3 Hz), 6.55 (1H, d, J=2.3 Hz), 6.68 (1H, d, J=7.9 Hz), 7.02 (1H, d, J=8.3 Hz), 7.08 (1H, d, J=6.1 Hz), 7.25 (1H, s), 7.38 (1H, s), 7.45 (1H, d, J=8.4 Hz), 7.52 (1H, d, J=7.6 Hz), 7.73 (1H, d, J=6.1 Hz)

### Intermediate 28

### 4-((1-(((1-Aminoisoquinolin-5-yl)amino)methyl)-2-azabicyclo[2.1.1]hexan-4-yl)methoxy)-1-methylpyridin-2(1H)-one

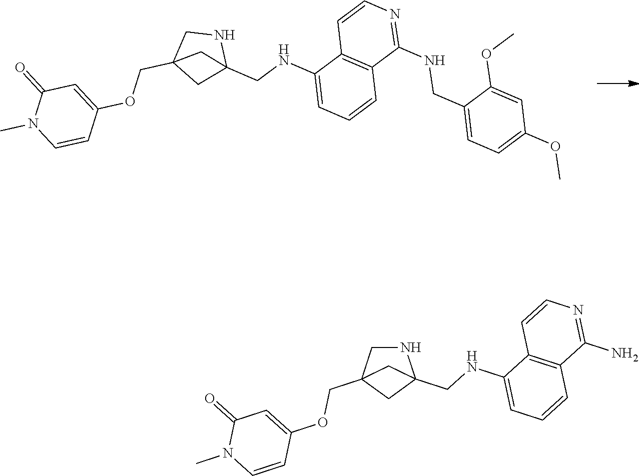

According to General Method 8, 4-((1-(((1-((2,4-dimethoxybenzyl)amino)isoquinolin-5-yl)amino)methyl)-2-azabicyclo[2.1.1]hexan-4-yl)methoxy)-1-methylpyridin-2(1H)-one (553 mg, 0.935 mmol) was deprotected in DCM (7.0 mL) at rt for 18 h. The reaction mixture was diluted with Et₂O (25 mL), stirred for 15 min and the resulting precipitate filtered-off and triturated with Et₂O (3×25 mL). The solid was diluted with MeOH (30 mL) and Amberlite IRN78 (10 mL) was added. The mixture was stirred for 2 h and filtered. The resin was washed with MeOH (100 mL). The filtrate was concentrated to afford the product (281 mg, 77% yield).

[M+H]⁺=392.3

¹H NMR (DMSO-d6, 400 MHz) δ 1.33-1.43 (2H, m), 1.57-1.67 (2H, m), 2.54 (1H, s), 2.83 (2H, s), 3.31 (3H, s), 3.40 (2H, d, J=5.4 Hz), 4.14 (2H, s), 5.74-5.81 (2H, m), 5.89 (1H, dd, J=7.5, 2.8 Hz), 6.48 (2H, s), 6.64 (1H, dd, J=7.7, 0.9 Hz), 7.10 (1H, dd, J=6.3, 0.9 Hz), 7.20 (1H, t, J=8.0 Hz), 7.32 (1H, d, J=8.3 Hz), 7.52 (1H, d, J=7.6 Hz), 7.72 (1H, d, J=6.1 Hz)

### Intermediate 29

### 4-((1-(((1-((2,4-Dimethoxybenzyl)amino)isoquinolin-5-yl)amino)methyl)-2-azabicyclo[2.1.1]hexan-4-yl)methoxy)-1,6-dimethylpyridin-2(1H)-one

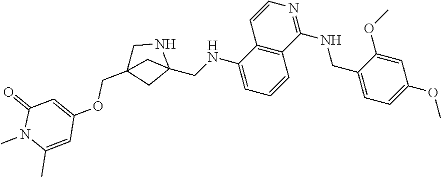

### tert-Butyl 4-(((1,6-dimethyl-2-oxo-1,2-dihydropyridin-4-yl)oxy)methyl)-1-formyl-2-azabicyclo[2.1.1]hexane-2-carboxylate

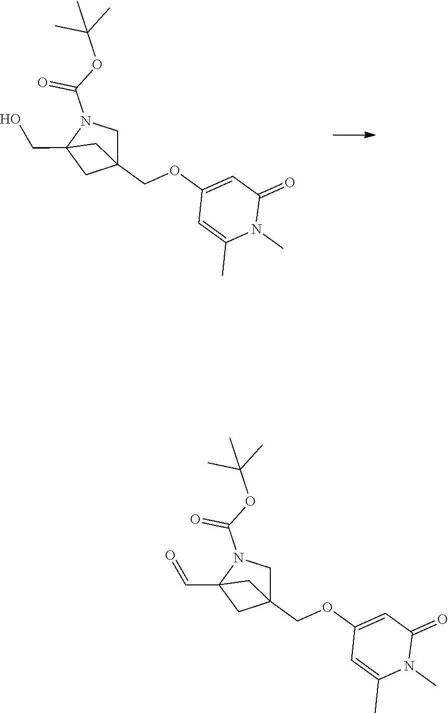 [SMILES: Cc1cc(OCC23CN(C(=O)OC(C)(C)C)C(CO)(C2)C3)cc(=O)n1C ; Cc1cc(OCC23CN(C(=O)OC(C)(C)C)C(C=O)(C2)C3)cc(=O)n1C]

Following General Method 16, tert-butyl 4-[(1,2-dimethyl-6-oxo-4-pyridyl)oxymethyl]-1-(hydroxymethyl)-2-azabicyclo[2.1.1]hexane-2-carboxylate (6500 mg, 17.8 mmol) was oxidised. Aqueous work up afforded the product (6.28 g, 78% yield, 80% pure).

¹H NMR (CDCl₃, 400 MHz) δ 1.46 (9H, s), 1.78 (2H, dd, J=4.6, 1.9 Hz), 2.11 (2H, dd, J=4.6, 1.8 Hz), 2.30 (3H, s), 3.45 (3H, s), 3.47 (2H, s), 4.06 (2H, s), 5.76 (1H, dt, J=2.8, 0.9 Hz), 5.83 (1H, d, J=2.8 Hz), 9.83 (1H, s)

### tert-Butyl 1-(((1-((2,4-dimethoxybenzyl)amino)isoquinolin-5-yl)amino)methyl)-4-(((1,6-dimethyl-2-oxo-1,2-dihydropyridin-4-yl)oxy)methyl)-2-azabicyclo[2.1.1]hexane-2-carboxylate

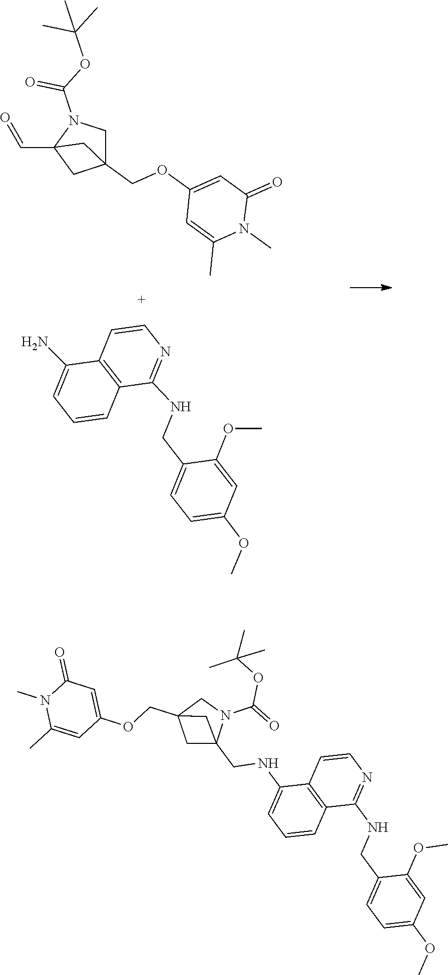

According to General Method 17, tert-butyl 4-[(1,2-dimethyl-6-oxo-4-pyridyl)oxymethyl]-1-formyl-2-azabicyclo[2.1.1]hexane-2-carboxylate (6.28 g, 13.9 mmol) was reacted with N1-[(2,4-dimethoxyphenyl)methyl]isoquinoline-1,5-diamine (4.99 g, 15.3 mmol) for 72 h. The reaction was quenched with a mixture of sat. NaHCO₃(aq) and brine (1:1 v/v, 40 mL). The mixture was stirred for 30 min at rt and the phases were separated. The aqueous phase was extracted with DCM (3×40 mL), and the combined organic layers were dried (Na₂SO₄), filtered, and concentrated. Purification by flash chromatography (Silica, 0-9% MeOH in EtOAc) afforded the product (2.90 g, 63% yield).

[M+H]⁺=655.8

¹H NMR (DMSO-d6, 400 MHz) δ 1.44 (3H, s), 1.48 (10H, s), 1.53 (2H, d, J=4.9 Hz), 1.91 (3H, s), 2.03 (1H, s), 2.29 (3H, s), 2.33 (6H, t, J=0.7 Hz), 3.34 (3H, s), 3.36 (8H, d, J=2.7 Hz), 3.73 (4H, s), 3.75 (3H, s), 3.87 (3H, s), 3.95 (2H, d, J=5.9 Hz), 4.13 (2H, s), 4.63 (2H, d, J=5.4 Hz), 5.67-5.78 (3H, m), 5.84-5.96 (3H, m), 6.07 (1H, s), 6.43 (1H, dd, J=8.4, 2.4 Hz), 6.59 (1H, d, J=2.4 Hz), 6.77 (1H, d, J=7.9 Hz), 6.99 (1H, d, J=6.2 Hz), 7.07 (1H, d, J=8.3 Hz), 7.30 (1H, t, J=8.0 Hz), 7.45 (1H, d, J=5.9 Hz), 7.51 (1H, d, J=8.4 Hz), 7.77 (1H, d, J=6.1 Hz)

### 4-((1-(((1-((2,4-Dimethoxybenzyl)amino)isoquinolin-5-yl)amino)methyl)-2-azabicyclo[2.1.1]hexan-4-yl)methoxy)-1,6-dimethylpyridin-2(1H)-one

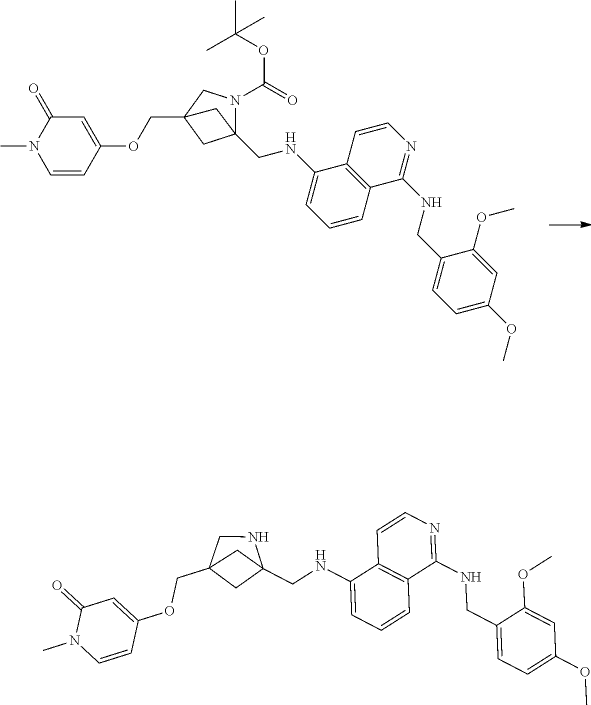

According to a variation of General Method 5a, HCl (4M in 1,4-dioxane, 26.7 mL, 107 mmol) was added to a mixture of tert-butyl 1-(((1-((2,4-dimethoxybenzyl)amino)isoquinolin-5-yl)amino)methyl)-4-(((1,6-dimethyl-2-oxo-1,2-dihydropyridin-4-yl)oxy)methyl)-2-azabicyclo[2.1.1]hexane-2-carboxylate (7.00 g, 10.7 mmol) in anhydrous THF (55 mL) at 0 °C. The mixture was warmed to rt, stirred for 18 h and filtered washing with Et₂O (5×25 mL) to afford the product as an HCl salt. The resulting solid was taken up in a mixture of DCM and MeOH (9:1 v/v, 200 mL) and sat. NaHCO₃ (aq) (125 mL, pH 9). The phases were separated, and the aqueous phase was extracted with a mixture of DCM and MeOH (9:1, 2×150 mL). The combined organic phases were dried (Na₂SO₄), filtered, and concentrated. Purification by flash chromatography (Silica, 0-20% (5% Et₃N in MeOH)) in DCM) afforded the product (3.30 g, 53% yield).

[M+H]⁺=556.8

¹H NMR (DMSO-d6, 400 MHz) δ 1.41 (2H, s), 1.66 (2H, d, J=3.8 Hz), 2.30 (3H, s), 2.87 (2H, s), 3.20 (1H, d, J=5.0 Hz), 3.34 (3H, s), 3.38 (2H, s), 3.45 (2H, d, J=5.3 Hz), 3.75 (3H, s), 3.86 (3H, s), 4.15 (2H, s), 4.62 (2H, d, J=5.4 Hz), 5.72 (1H, d, J=2.8 Hz), 5.89 (1H, dd, J=2.8, 0.9 Hz), 5.93 (1H, t, J=5.6 Hz), 6.42 (1H, dd, J=8.4, 2.4 Hz), 6.59 (1H, d, J=2.4 Hz), 6.70 (1H, d, J=7.8 Hz), 7.05 (1H, d, J=8.3 Hz), 7.12 (1H, d, J=6.1 Hz), δ 7.28 (1H, t, J=8.0 Hz), 7.45 (1H, d, J=5.9 Hz), 7.49 (1H, d, J=8.5 Hz), 7.76 (1H, d, J=6.1 Hz)

### Intermediate 30

### 4-((1-(((1-Aminoisoquinolin-5-yl)amino)methyl)-2-azabicyclo[2.1.1]hexan-4-yl)methoxy)-1,6-dimethylpyridin-2(1H)-one

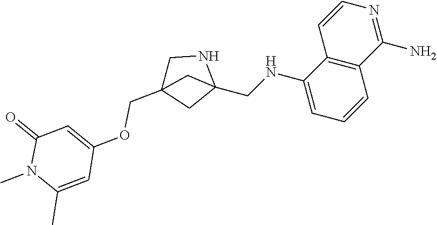 [SMILES: Cc1cc(OCC23CNC(CNc4cccc5c(N)nccc45)(C2)C3)cc(=O)n1C]

### 4-((1-(((1-Aminoisoquinolin-5-yl)amino)methyl)-2-azabicyclo[2.1.1]hexan-4-yl)methoxy)-1,6-dimethylpyridin-2(1H)-one

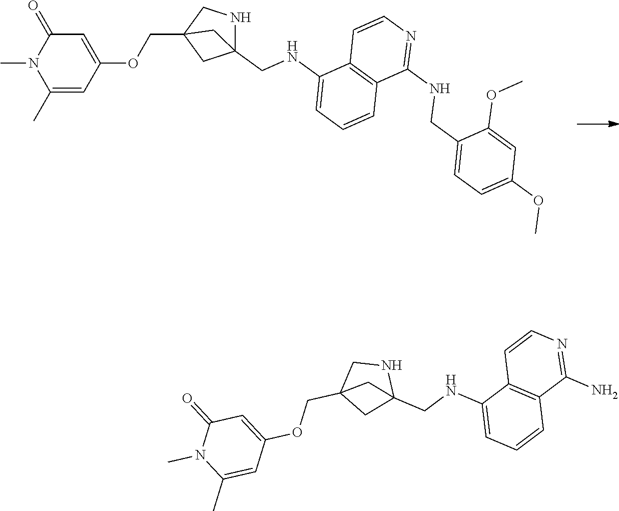 [SMILES: COc1ccc(CNc2nccc3c(NCC45CC(COc6cc(C)n(C)c(=O)c6)(CN4)C5)cccc23)c(OC)c1 ; Cc1cc(OCC23CNC(CNc4cccc5c(N)nccc45)(C2)C3)cc(=O)n1C]

According to General Method 8, 4-((1-(((1-((2,4-dimethoxybenzyl)amino)isoquinolin-5-yl)amino)methyl)-2-azabicyclo[2.1.1]hexan-4-yl)methoxy)-1,6-dimethylpyridin-2(1H)-one (1.51 g, 12.58 mmol) was deprotected in DCM (25 mL) at rt for 18 h. The mixture was diluted with Et₂O (75 mL), stirred for 15 min and the precipitate was isolated by filtration and triturated with Et₂O (3×25 mL). The solid was taken up in MeOH (25 mL) and Amberlite IRN78 (20 mL) was added. The mixture was stirred for 2 h and filtered. The resin was washed with MeOH (100 mL) and the filtrate concentrated to afford the product (889 mg, 85% yield).

[M+H]⁺=406.2

¹H NMR (DMSO-d6, 400 MHz) δ 1.32-1.43 (2H, m), 1.58-1.64 (2H, m), 2.26 (3H, s), 2.55 (1H, t, J=6.2 Hz), 2.82 (2H, d, J=5.3 Hz), 3.30 (3H, s), 3.40 (2H, d, J=5.5 Hz), 4.11 (2H, s), 5.68 (1H, d, J=2.8 Hz), 5.80 (1H, t, J=5.6 Hz), 5.85 (1H, dd, J=2.8, 0.9 Hz), 6.50 (2H, s), 6.64 (1H, d, J=7.7 Hz), 7.11 (1H, d, J=6.1 Hz), 7.20 (1H, t, J=8.0 Hz), 7.33 (1H, d, J=8.3 Hz), 7.73 (1H, d, J=6.1 Hz)

### Intermediate 31

### N5-((4-(((tert-Butyldiphenylsilyl)oxy)methyl)-2-azabicyclo[2.1.1]hexan-1-yl)methyl)-N1-(2,4-dimethoxybenzyl)isoquinoline-1,5-diamine

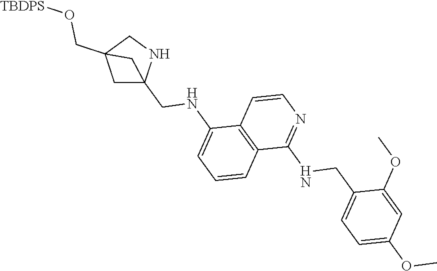

### tert-Butyl 4-(((tert-butyldiphenylsilyl)oxy)methyl)-1-(hydroxymethyl)-2-azabicyclo[2.1.1]hexane-2-carboxylate

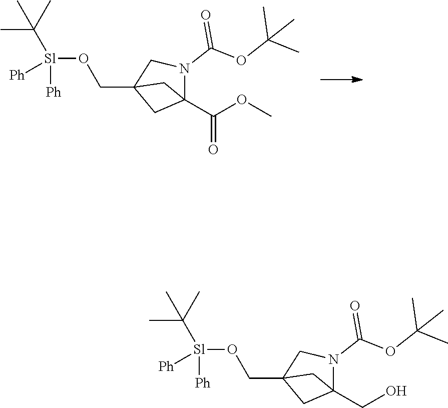

According to a variation of General Method 3b, 2-(tert-butyl) 1-methyl 4-(((tert-butyldiphenylsilyl)oxy)methyl)-2-azabicyclo[2.1.1]hexane-1,2-dicarboxylate (90%, 59.5 g, 105 mmol) was reduced using LiBH₄ (8.00 g, 330 mmol). The mixture was cooled to 0 °C and quenched with sat. NH₄Cl (aq) (350 mL), H₂O (500 mL), and EtOAc (500 mL). The layers were separated, and the aqueous layer was extracted with EtOAc (2×500 mL). The combined organic layers were dried (Na₂SO₄), filtered, and concentrated to afford the product (51.1 g, 91% yield).

[M−tBu+H]⁺=426.4

¹H NMR (CDCl3, 300 MHz) δ 1.06 (9H, s), 1.47 (9H, s), 3.25 (2H, s), 3.80 (2H, s), 3.91 (2H, s), 7.32-7.47 (6H, m), 7.57-7.68 (4H, m)

### tert-Butyl 4-(((tert-butyldiphenylsilyl)oxy)methyl)-1-(((1-((2,4-dimethoxybenzyl)amino)isoquinolin-5-yl)amino)methyl)-2-azabicyclo[2.1.1]hexane-2-carboxylate

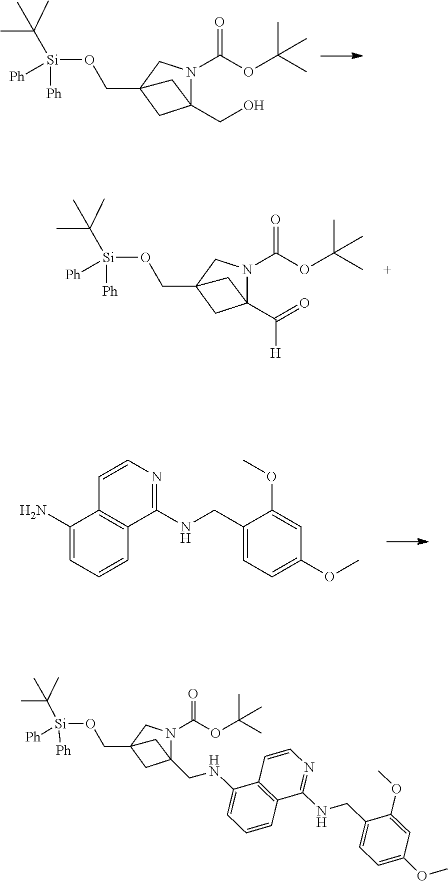

Following General Method 16, tert-butyl 4-(((tert-butyldiphenylsilyl)oxy)methyl)-1-(hydroxymethyl)-2-azabicyclo[2.1.1]hexane-2-carboxylate (7.00 g, 13.1 mmol, 90% purity) was oxidised for 16 h. Aqueous work up afforded the intermediate tert-butyl 4-(((tert-butyldiphenylsilyl)oxy)methyl)-1-formyl-2-azabicyclo[2.1.1]hexane-2-carboxylate which was reacted directly with N1-(2,4-dimethoxybenzyl)isoquinoline-1,5-diamine (4.50 mg, 14.5 mmol) following General Method 17 for 16 h. Aqueous work up and purification by flash chromatography (Silica, 0-52% EtOAc in hexanes) afforded the product (4.05 g, 40% yield).

[M+H]⁺=774.8

¹H NMR (DMSO-d6, 300 MHz) δ 0.93 (9H, s), 1.41 (11H, s), 1.78 (2H, s), 3.22 (2H, s), 3.70 (3H, s), 3.78 (2H, s), 3.82 (3H, s), 3.88 (2H, d, J=5.5 Hz), 4.59 (2H, d, J=5.6 Hz), 5.96 (1H, s), 6.36 (1H, d, J=8.4 Hz), 6.55 (1H, d, J=2.3 Hz), 6.71 (1H, d, J=7.8 Hz), 6.95 (1H, d, J=6.4 Hz), 7.02 (1H, d, J=8.4 Hz), 7.26 (1H, t, J=8.0 Hz), 7.34-7.49 (8H, m), 7.53 (4H, d, J=7.6 Hz), 7.72 (1H, d, J=6.1 Hz)

### N5-((4-(((tert-Butyldiphenylsilyl)oxy)methyl)-2-azabicyclo[2.1.1]hexan-1-yl)methyl)-N1-(2,4-dimethoxybenzyl)isoquinoline-1,5-diamine

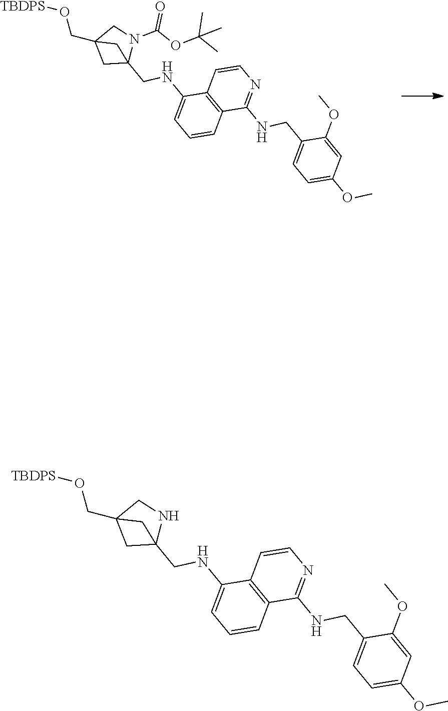

Following General Method 5a, tert-butyl 4-(((tert-butyldiphenylsilyl)oxy)methyl)-1-(((1-((2,4-dimethoxybenzyl)amino)isoquinolin-5-yl)amino)methyl)-2-azabicyclo[2.1.1]hexane-2-carboxylate (4.05 g, 4.72 mmol) was deprotected in THF (30 mL) at rt for 72 h. After concentration and trituration with Et₂O, the residue was diluted with NaOH (aq) (1N, 100 mL) and 1:8 MeOH:DCM (100 mL). The layers were separated and extracted with 1:8 MeOH/DCM (3×75 mL). The combined organic layers were washed with brine (200 mL), dried (Na₂SO₄), filtered, and concentrated. Purification by flash chromatography (Silica, 0-10% (10% NH₄OH in MeOH) in DCM) afforded the product (1.3 g, 41% yield).

[M+H]⁺=673.5

¹H NMR (DMSO-d6, 300 MHz) δ 0.97 (9H, s), 1.29-1.37 (2H, m), 1.49-1.57 (2H, m), 2.77 (2H, s), 3.39 (2H, d, J=5.3 Hz), 3.71 (3H, s), 3.83 (5H, s), 4.59 (2H, d, J=5.4 Hz), 5.72-5.77 (1H, m), 6.34-6.42 (1H, m), 6.55 (1H, d, J=2.0 Hz), 6.67 (1H, d, J=7.8 Hz), 7.03 (1H, d, J=8.3 Hz), 7.07 (1H, d, J=6.0 Hz), 7.25 (1H, t, J=8.1 Hz), 7.35-7.49 (8H, m), 7.54-7.61 (4H, m), 7.73 (1H, d, J=6.0 Hz)

### Intermediate 32

### 4-(((1,6-Dimethyl-2-oxo-1,2-dihydropyridin-4-yl)oxy)methyl)-2-(5-methoxybenzo[d]oxazol-2-yl)-2-azabicyclo[2.1.1]hexane-1-carbaldehyde

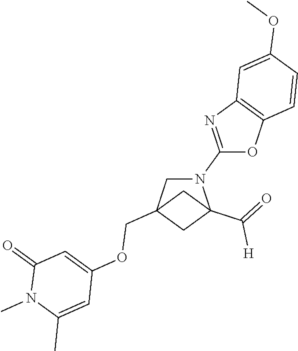 [SMILES: COc1ccc2oc(N3CC4(COc5cc(C)n(C)c(=O)c5)CC3(C=O)C4)nc2c1]

### 4-((1-(Hydroxymethyl)-2-azabicyclo[2.1.1]hexan-4-yl)methoxy)-1,6-dimethylpyridin-2(1H)-one

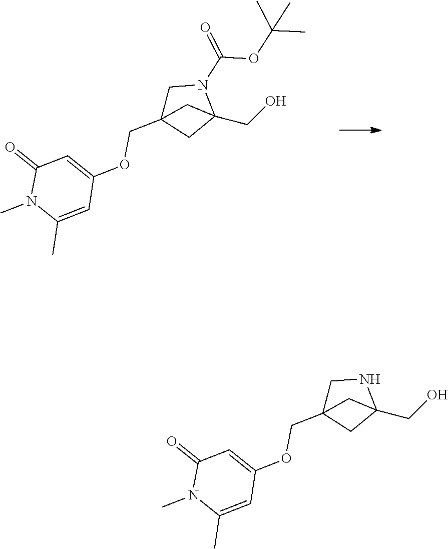

Following general Method 5a, tert-butyl 4-[(1,2-dimethyl-6-oxopyridin-4-yl)oxymethyl]-1-(hydroxymethyl)-2-azabicyclo[2.1.1]hexane-2-carboxylate (2.10 g, 5.76 mmol) was deprotected at rt for 18 h. Concentration afforded the product (1.70 g, 98% yield).

[M+H]⁺=265.1

### 4-((1-(Hydroxymethyl)-2-(5-methoxybenzo[d]oxazol-2-yl)-2-azabicyclo[2.1.1]hexan-4-yl)methoxy)-1,6-dimethylpyridin-2(1H)-one

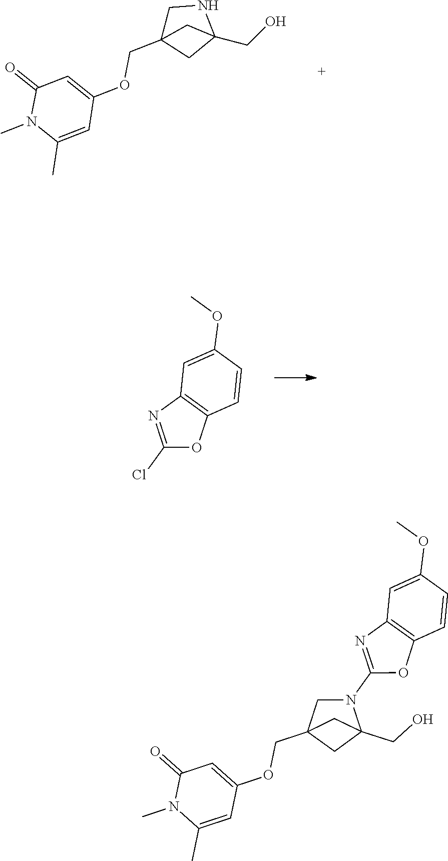 [SMILES: Cc1cc(OCC23CNC(CO)(C2)C3)cc(=O)n1C ; COc1ccc2oc(Cl)nc2c1 ; COc1ccc2oc(N3CC4(COc5cc(C)n(C)c(=O)c5)CC3(CO)C4)nc2c1]

According to General Method 2b, 4-[[1-(hydroxymethyl)-2-azabicyclo[2.1.1]hexan-4-yl]methoxy]-1,6-dimethylpyridin-2-one hydrochloride (1.50 mg, 4.99 mmol) was reacted with 2-chloro-5-methoxybenzo[d]oxazole (1.10 mg, 5.98 mmol) in DMSO (3 mL) using K₂CO₃ (2.76 mg, 19.95 mmol), at rt for 3 days. The solvent was removed (Biotage V10) and the residue was partitioned between DCM (50 mL) and water (50 mL). The organic phase was dried (MgSO₄) and concentrated to afford the product (2.00 g, 97% yield).

[M+H]⁺=412.2

### 4-(((1,6-Dimethyl-2-oxo-1,2-dihydropyridin-4-yl)oxy)methyl)-2-(5-methoxybenzo[d]oxazol-2-yl)-2-azabicyclo[2.1.1]hexane-1-carbaldehyde

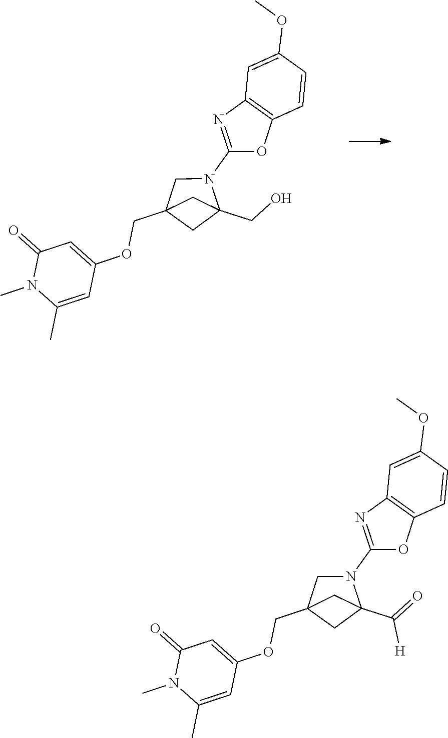 [SMILES: COc1ccc2oc(N3CC4(COc5cc(C)n(C)c(=O)c5)CC3(CO)C4)nc2c1 ; COc1ccc2oc(N3CC4(COc5cc(C)n(C)c(=O)c5)CC3(C=O)C4)nc2c1]

Following General Method 16, 4-[[1-(hydroxymethyl)-2-(5-methoxy-1,3-benzoxazol-2-yl)-2-azabicyclo[2.1.1]hexan-4-yl]methoxy]-1,6-dimethylpyridin-2-one (200 mg, 0.49 mmol) was oxidised. After quenching with 1:1 sat. Na₂S2O₃ (aq) (10 mL) and sat. NaHCO₃ (aq) (10 mL), aqueous work up afforded the product (195 mg, 98% yield).

[M+H]⁺=410.1

### Intermediate 33

### N1-[(2,4-Dimethoxyphenyl)methyl]-N5-methyl-isoquinoline-1,5-diamine

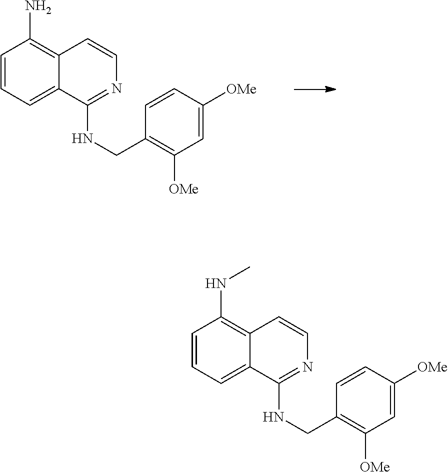 [SMILES: COc1ccc(CNc2nccc3c(N)cccc23)c(OC)c1 ; CNc1cccc2c(NCc3ccc(OC)cc3OC)nccc12]

Copper(II) acetate (304 mg, 1.59 mmol) was added to a solution of N1-[(2,4-dimethoxyphenyl)methyl]isoquinoline-1,5-diamine (249 mg, 0.645 mmol) and pyridine (180 μL, 2.21 mmol) in 1,4-dioxane (8 mL) at rt. The mixture was stirred for 10 min at rt and methylboronic acid (98.8 mg, 1.62 mmol) was added. The mixture was heated at reflux for 18 h, cooled to rt, filtered over Celite® and washed with EtOAc (3×20 mL). The filtrate was concentrated, and the residue was purified by flash chromatography (silica, 0-88% EtOAc in hexanes) to afford the product (127 mg, 61%).

[M+H]⁺=324.2

¹H NMR (DMSO-d6, 400 MHz) δ 2.81 (3H, d, J=4.8 Hz), 3.71 (3H, s), 3.82 (3H, s), 4.59 (2H, d, J=5.6 Hz), 6.12 (1H, q, J=4.9 Hz), 6.39 (1H, dd, J=8.3, 2.4 Hz), 6.52-6.58 (2H, m), 7.02 (1H, d, J=2.7 Hz), 7.04 (1H, d, J=0.8 Hz), 7.28 (1H, t, J=8.0 Hz), 7.36 (1H, t, J=5.9 Hz), 7.44 (1H, d, J=8.4 Hz), 7.72 (1H, d, J=6.0 Hz)

### Specific Examples of the Present Invention

### Example Number 1

### N5-((4-(5-Phenyloxazol-2-yl)-2-oxabicyclo[2.1.1]hexan-1-yl)methyl)isoquinoline-1,5-diamine

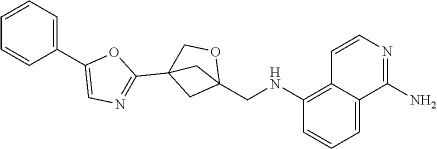 [SMILES: Nc1nccc2c(NCC34CC(c5ncc(-c6ccccc6)o5)(CO3)C4)cccc12]

### tert-Butyl ((4-(hydroxymethyl)-2-oxabicyclo[2.1.1]hexan-1-yl)methyl)carbamate

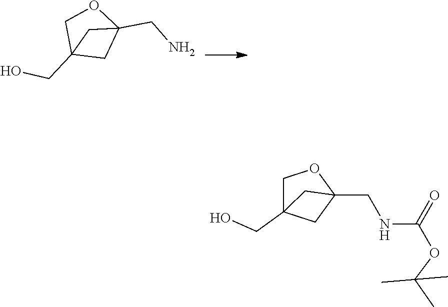 [SMILES: NCC12CC(CO)(CO1)C2 ; CC(C)(C)OC(=O)NCC12CC(CO)(CO1)C2]

To a solution of [1-(aminomethyl)-2-oxabicyclo[2.1.1]hexan-4-yl]methanol hydrochloride (1.26 g, 7.01 mmol) and TEA (2.50 mL, 17.94 mmol) in THF (35 mL) at 0 °C was added Boc₂O (2.00 g, 9.16 mmol) and the mixture was stirred for 10 min at 0 °C. The reaction was then stirred for 18 h at rt. The reaction was quenched with water (30 mL) and washed with EtOAc (2×50 mL), dried (MgSO₄) and concentrated. Purification by flash chromatography (Silica, 0-20% MeOH in DCM) afforded the product (1.29 g 75% yield).

[M+Na]⁺=266.2

¹H NMR (DMSO-d6, 400 MHz) δ 1.26-1.34 (2H, m), 1.37 (9H, s), 1.52-1.66 (2H, m), 3.18 (2H, d, J=6.1 Hz), 3.51 (2H, s), 3.61 (2H, d, J=5.4 Hz), 4.62 (1H, t, J=5.5 Hz), 6.84 (1H, t, J=6.1 Hz)

### 1-(((tert-Butoxycarbonyl)amino)methyl)-2-oxabicyclo[2.1.1]hexane-4-carboxylic acid

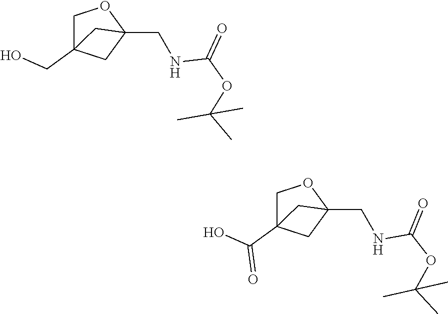 [SMILES: CC(C)(C)OC(=O)NCC12CC(CO)(CO1)C2 ; CC(C)(C)OC(=O)NCC12CC(C(=O)O)(CO1)C2]

To a mixture of tert-butyl N-[[4-(hydroxymethyl)-2-oxabicyclo[2.1.1]hexan-1-yl]methyl]carbamate (500 mg, 2.06 mmol) and NaIO₄ (1758 mg, 8.22 mmol) in CCl₄ (4 mL), MeCN (4 mL) and H₂O (6 mL) was added RuCl₃·H₂O (15 mg, 0.07 mmol). The mixture was stirred at rt for 2 h and diluted with EtOAc (25 mL) and sat. NH₄Cl (aq) (25 mL). The aqueous phase was extracted with EtOAc (3×20 mL) and the combined organic phases were washed brine (50 mL), dried (Na₂SO₄), filtered and concentrated. The residue was diluted in EtOAc (10 mL) and filtered on a Florisil pad. The filtrate was concentrated to afford the product (390 mg, 74% yield).

¹H NMR (DMSO-d6, 300 MHz) δ 1.38 (9H, s), 1.61 (2H, d, J=4.2 Hz), 1.92 (2H, d, J=4.0 Hz), 3.20 (2H, d, J=6.0 Hz), 3.74 (2H, s), 6.92 (1H, t, J=5.9 Hz), 12.66 (1H, s)

### Tert-butyl ((4-((2-oxo-2-phenylethyl)carbamoyl)-2-oxabicyclo[2.1.1]hexan-1-yl)methyl)carbamate

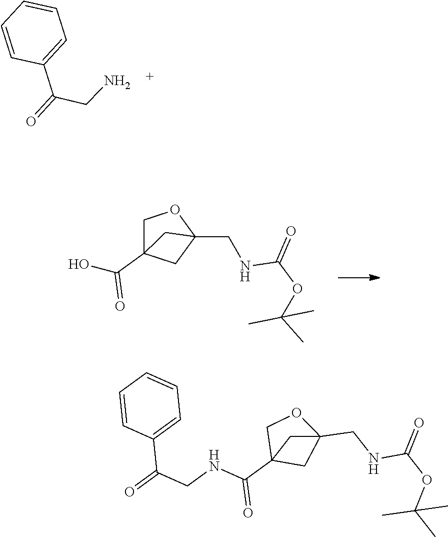 [SMILES: NCC(=O)c1ccccc1 ; CC(C)(C)OC(=O)NCC12CC(C(=O)O)(CO1)C2 ; CC(C)(C)OC(=O)NCC12CC(C(=O)NCC(=O)c3ccccc3)(CO1)C2]

According to General Method 18b, 1-[(tert-butoxycarbonylamino)methyl]-2-oxabicyclo[2.1.1]hexane-4-carboxylic acid (130 mg, 0.51 mmol) was coupled to 2-amino-1-phenyl-ethanone hydrochloride (87 mg, 0.51 mmol). After aqueous work up, purification by flash chromatography (Silica, 0-90% EtOAc in hexanes) afforded the product (137 mg, 72% yield).

[M−H]⁻ 373.4

¹H NMR (CDCl₃, 300 MHz) δ 1.45 (9H, s), 1.95 (2H, d, J=4.4 Hz), 2.05 (2H, d, J=4.5 Hz), 3.44-3.54 (2H, m), 3.97 (2H, s), 4.72-4.86 (3H, m), 6.70 (1H, s), 7.52 (2H, t, J=7.6 Hz), 7.65 (1H, t, J=7.3 Hz), 7.99 (2H, d, J=7.6 Hz)

### (4-(5-Phenyloxazol-2-yl)-2-oxabicyclo[2.1.1]hexan-1-yl)methanamine

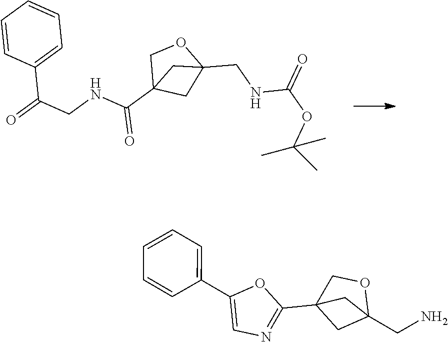 [SMILES: CC(C)(C)OC(=O)NCC12CC(C(=O)NCC(=O)c3ccccc3)(CO1)C2 ; NCC12CC(c3ncc(-c4ccccc4)o3)(CO1)C2]

A mixture of tert-butyl N-[[4-(phenacylcarbamoyl)-2-oxabicyclo[2.1.1]hexan-1-yl]methyl]carbamate (120 mg, 0.32 mmol) in 1M H₂SO₄ (aq) (1 mL) was stirred at rt for 1 h. The mixture was basified with a solution of 1M NH₄OH (aq) and extracted with EtOAc (3×10 mL). The combined organic layers were dried (Na₂SO₄), filtered and concentrated to afford the product (52 mg, 38% yield).

[M+H]⁺257.3

### N5-((4-(5-Phenyloxazol-2-yl)-2-oxabicyclo[2.1.1]hexan-1-yl)methyl)isoquinoline-1,5-diamine

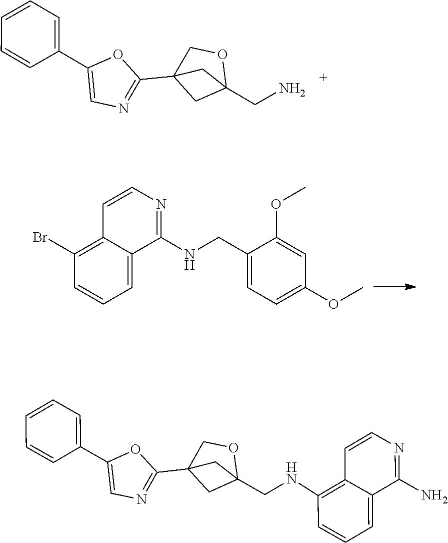 [SMILES: NCC12CC(c3ncc(-c4ccccc4)o3)(CO1)C2 ; COc1ccc(CNc2nccc3c(Br)cccc23)c(OC)c1 ; Nc1nccc2c(NCC34CC(c5ncc(-c6ccccc6)o5)(CO3)C4)cccc12]

Following a variation on General Method 4, [4-(5-phenyloxazol-2-yl)-2-oxabicyclo[2.1.1]hexan-1-yl]methanamine (50 mg, 0.12 mmol, 60% pure) was coupled to 5-bromo-N-(2,4-dimethoxybenzyl)isoquinolin-1-amine (53 mg, 0.14 mmol) in the presence of NaO^{t}Bu (30 mg, 0.31 mmol) and Pd-175 (15 mg, 0.02 mmol) in 1,4-dioxane at 100 °C for 16 h. After cooling to rt, the reaction mixture was filtered over Celite® washing with EtOAc (3×25 mL) and the filtrate was concentrated to afford the intermediate N1-[(2,4-dimethoxyphenyl)methyl]-N5-[[4-(5-phenyloxazol-2-yl)-2-oxabicyclo[2.1.1]hexan-1-yl]methyl]isoquinoline-1,5-diamine.

[M+H]⁺=549.3

Deprotection was performed according to a variation of General Method 8, in DCM (2 mL) at rt for 3 h. After concentration and aqueous work up, purification by flash chromatography (C18, 10-60% MeCN in 10 mM NH₄HCO₃ (aq)), followed by automated prep HPLC (mass directed, 42-52% MeCN in 10 mM NH₄HCO₃ (aq) over 9 min) afforded the product (11 mg, 24% yield).

[M+H]⁺=399.3

¹H NMR (DMSO-d6, 300 MHz) δ 1.94 (2H, d, J=4.2 Hz), 2.32 (2H, d, J=4.4 Hz), 3.63 (2H, d, J=5.4 Hz), 4.06 (2H, s), 6.00 (1H, t, J=5.8 Hz), 6.51 (2H, s), 6.74 (1H, d, J=7.7 Hz), 7.14 (1H, d, J=6.1 Hz), 7.23 (1H, t, J=7.9 Hz), 7.32-7.40 (2H, m), 7.41-7.50 (2H, m), 7.63 (1H, s), 7.66-7.77 (3H, m)

### Example Number 15

### 4-((1-(((1-Aminoisoquinolin-5-yl)amino)methyl)-2-(2-(chroman-6-yl)acetyl)-2-azabicyclo[2.1.1]hexan-4-yl)methoxy)-1-methylpyridin-2(1H)-one

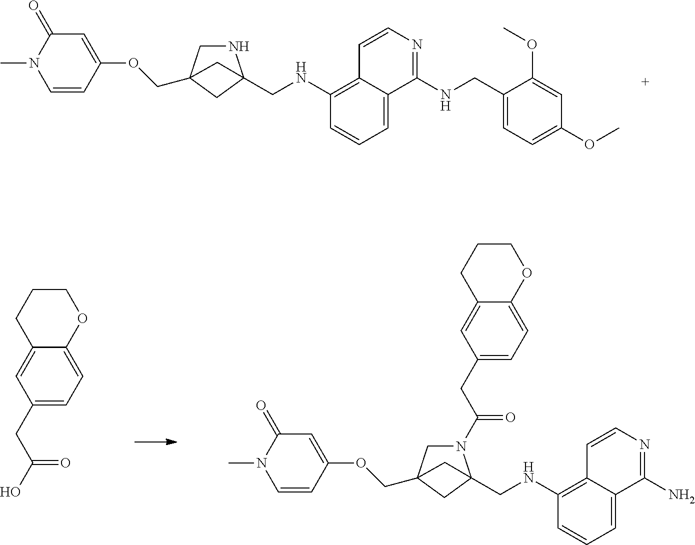

According to General Method 18a, 2-chroman-6-ylacetic acid (28 mg, 0.14 mmol) was coupled to 4-((1-(((1-((2,4-dimethoxybenzyl)amino)isoquinolin-5-yl)amino)methyl)-2-azabicyclo[2.1.1]hexan-4-yl)methoxy)-1-methylpyridin-2(1H)-one (61 mg, 0.11 mmol). Concentration afforded 4-((2-(2-(chroman-6-yl)acetyl)-1-(((1-((2,4-dimethoxybenzyl)amino)isoquinolin-5-yl)amino)methyl)-2-azabicyclo[2.1.1]hexan-4-yl)methoxyl-1-methylpyridin-2(1H)-one which was deprotected according to General Method 8 in DCM (1 mL) at rt for 18 h. Purification by flash chromatography (C18, 20-47% MeCN in 10 mM (NH₄)HCO₃ (aq)) provided the product (41 mg, 65% yield).

[M+H]⁺=566.6

¹H NMR (DMSO-d6, 500 MHz) δ 1.47-1.53 (2H, m), 1.87-1.94 (4H, m), 2.69 (2H, t, J=6.4 Hz), 3.31 (3H, s), 3.52 (2H, s), 3.56 (2H, s), 3.98 (2H, d, J=6.6 Hz), 4.07-4.13 (2H, m), 4.14 (2H, s), 5.78 (1H, d, J=2.7 Hz), 5.88 (1H, dd, J=7.5, 2.8 Hz), 6.16 (1H, t, J=6.6 Hz), 6.51 (2H, d, J=5.3 Hz), 6.66 (1H, d, J=8.1 Hz), 6.79 (2H, t, J=7.5 Hz), 6.97 (2H, d, J=8.9 Hz), 7.21 (1H, t, J=8.0 Hz), 7.33 (1H, d, J=8.3 Hz), 7.54 (1H, d, J=7.5 Hz), 7.70 (1H, d, J=6.1 Hz)

### Example Numbers 24 and 25

### 4-((1-(((1-Aminoisoquinolin-5-yl)amino)methyl)-2-(2-trans-(-4-methoxycyclohexyl)acetyl)-2-azabicyclo[2.1.1]hexan-4-yl)methoxy)-1,6-dimethylpyridin-2(1H)-one

### And

### 4-((1-(((1-aminoisoquinolin-5-yl)amino)methyl)-2-(2-cis-(4-methoxycyclohexyl)acetyl)-2-azabicyclo[2.1.1]hexan-4-yl)methoxy)-1,6-dimethylpyridin-2(1H)-one

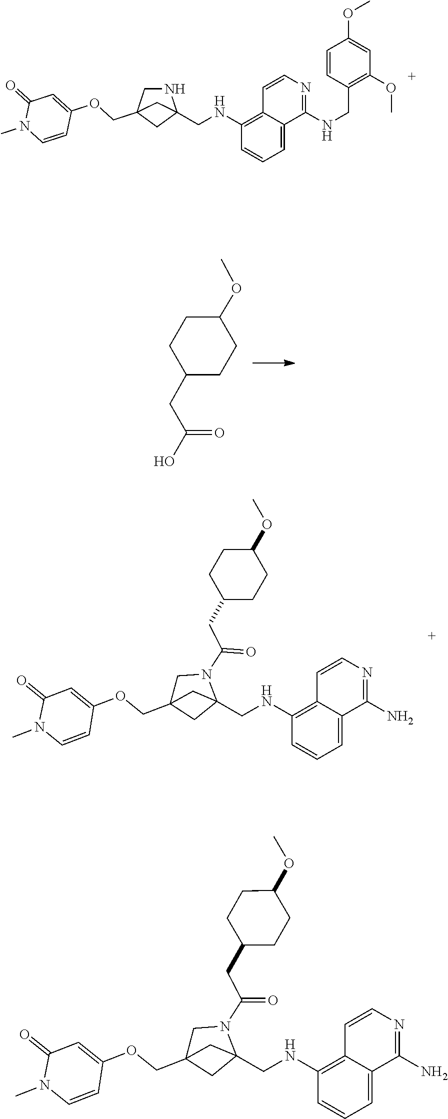

According to General Method 18a, 2-(4-methoxycyclohexyl)acetic acid (55 mg, 0.30 mmol) was coupled with 4-((1-(((1-((2,4-dimethoxybenzyl)amino)isoquinolin-5-yl)amino)methyl)-2-azabicyclo[2.1.1]hexan-4-yl)methoxy)-1-methylpyridin-2(1H)-one (154 mg, 0.26 mmol). Concentration afforded 4-((1-(((1-((2,4-dimethoxybenzyl)amino)isoquinolin-5-yl)amino)methyl)-2-(2-(4-methoxycyclohexyl)acetyl)-2-azabicyclo[2.1.1]hexan-4-yl)methoxy)-1-methylpyridin-2(1H)-one which was deprotected according to General Method 8 in DCM (4 mL) at rt for 18 h. Purification by flash chromatography (C18, 20-42% MeCN in 10 mM (NH₄)HCO₃ (aq)) followed by automated prep. HPLC (mass directed, 30-40% MeCN in 10 mM (NH₄)HCO₃ (aq) over 9 min) provided the product 4-((1-(((1-aminoisoquinolin-5-yl)amino)methyl)-2-(2-trans-(-4-methoxycyclohexyl)acetyl)-2-azabicyclo[2.1.1]hexan-4-yl)methoxy)-1,6-dimethylpyridin-2(1H)-one (46 mg, 31% yield) as the first eluting isomer.

[M+H]⁺=560.6

¹H NMR (DMSO-d6, 400 MHz) δ 0.93-1.17 (4H, m), 1.45-1.50 (2H, m), 1.71-1.81 (3H, m), 1.84-1.90 (2H, m), 1.93-2.01 (2H, m), 2.15 (2H, d, J=6.5 Hz), 2.26 (3H, s, 2.99-3.08 (1H, m), 3.22 (3H, s), 3.31 (3H, s), 3.46 (2H, s), 3.97 (2H, d, J=6.5 Hz), 4.10 (2H, s), 5.68 (1H, d, J=2.8 Hz), 5.84 (1H, dd, J=2.8, 0.9 Hz), 6.20 (1H, t, J=6.8 Hz), 6.52 (2H, s), 6.76-6.84 (2H, m), 7.20 (1H, t, J=8.0 Hz), 7.32 (1H, d, J=8.3 Hz), 7.70 (1H, d, J=6.1 Hz) and 4-((1-(((1-aminoisoquinolin-5-yl)amino)methyl)-2-(2-cis-(4-methoxycyclohexyl)acetyl)-2-azabicyclo[2.1.1]hexan-4-yl)methoxy)-1,6-dimethylpyridin-2(1H)-one (18 mg, 12% yield) as the second eluting iomer.

[M+H]⁺=560.7

¹H NMR (DMSO-d6, 500 MHz) δ 1.22-1.32 (2H, m), 1.37-1.51 (6H, m), 1.73-1.82 (2H, m), 1.83-1.92 (3H, m), 2.15 (2H, d, J=7.0 Hz), 2.26 (3H, s), 3.19 (3H, s), 3.31 (3H, s), 3.34-3.38 (1H, m), 3.48 (2H, s), 3.98 (2H, d, J=6.6 Hz), 4.10 (2H, s), 5.68 (1H, d, J=2.8 Hz), 5.84 (1H, dd, J=2.8, 0.9 Hz), 6.23 (1H, t, J=6.7 Hz), 6.60 (2H, s), 6.78-6.86 (2H, m), 7.22 (1H, t, J=8.0 Hz), 7.33 (1H, d, J=8.3 Hz), 7.70 (1H, d, J=6.0 Hz)

### Example Number 29

### N-(1-(1-(((1-Aminoisoquinolin-5-yl)amino)methyl)-4-(((1,6-dimethyl-2-oxo-1,2-dihydropyridin-4-yl)oxy)methyl)-2-azabicyclo[2.1.1]hexan-2-yl)-1-oxopropan-2-yl)acetamide

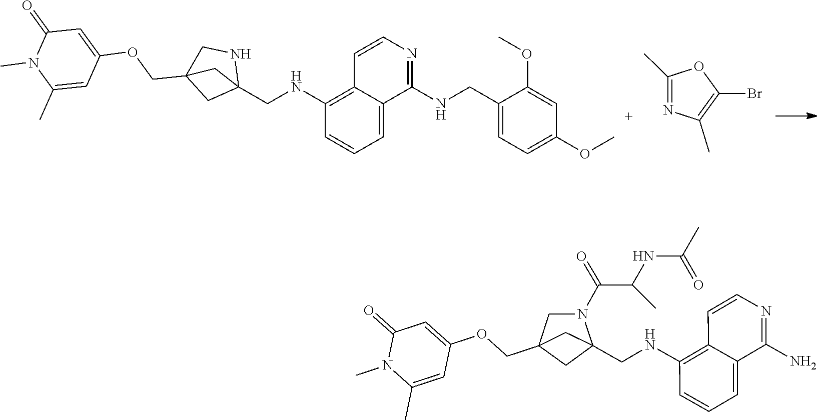

According to General Method 4, 4-((1-(((1-((2,4-dimethoxybenzyl)amino)isoquinolin-5-yl)amino)methyl)-2-azabicyclo[2.1.1]hexan-4-yl)methoxy)-1,6-dimethylpyridin-2(1H)-one (60 mg, 0.11 mmol) was coupled to 5-bromo-2,4-dimethyl-oxazole (24 mg, 0.13 mmol) using NaOtBu (27 mg, 0.27 mmol) and Pd-PEPPSI-Pent (9 mg, 0.01 mmol) in 1,4-dioxane (1 mL) at reflux for 24 h. 5-Bromo-2,4-dimethyl-oxazole (23.5 mg, 0.130 mmol) and Pd-PEPPSI-IPent (9 mg, 0.0108 mmol) were added and the mixture was refluxed for 48 h, cooled to rt, diluted with MeOH (500 μL), filtered washing with MeOH (5 mL) and concentrated to afford N-(1-(1-(((1-((2,4-dimethoxybenzyl)amino)isoquinolin-5-yl)amino)methyl)-4-(((1,6-dimethyl-2-oxo-1,2-dihydropyridin-4-yl)oxy)methyl)-2-azabicyclo[2.1.1]hexan-2-yl)-1-oxopropan-2-yl)acetamide which was deprotected according to General Method 8 in DCM (2 mL) at rt for 18 h. Flash chromatography (C18, 20-60% MeCN in 10 mM (NH₄)HCO₃ (aq)) followed by an automated prep HPLC (UV directed 30-40% MeCN in 10 mM (NH₄)HCO₃ (aq) over 8 min) provided the product (24 mg, 45% yield).

[M+H]⁺=518.8

¹H NMR (DMSO-d6, 400 MHz) δ 1.27 (3H, d, J=6.9 Hz), 1.53 (1H, d, J=3.4 Hz), 1.89 (3H, s), 1.92 (2H, t, J=5.9 Hz), 2.30 (3H, s), 3.34 (4H, s), 3.57 (1H, d, J=7.4 Hz), 3.72 (1H, d, J=7.5 Hz), 3.94-4.11 (2H, m), 4.17 (2H, s), 4.47 (1H, t, J=7.1 Hz), 5.73 (1H, d, J=2.8 Hz), 5.89 (1H, dd, J=2.9, 0.9 Hz), 6.16 (1H, t, J=6.5 Hz), 6.56 (2H, s), 6.82 (1H, d, J=7.7 Hz), 6.87 (1H, d, J=6.2 Hz), 7.25 (1H, t, J=8.0 Hz), 7.37 (1H, d, J=8.0 Hz), 7.74 (1H, d, J=8.0 Hz) 8.24 (1H, d, J=8.0 Hz)

### Example Number 30

### 1-(((1-Aminoisoquinolin-5-yl)amino)methyl)-4-(((1-methyl-2-oxo-1,2-dihydropyridin-4-yl)oxy)methyl)-N-(1-phenylcyclopropyl)-2-azabicyclo[2.1.1]hexane-2-carboxamide

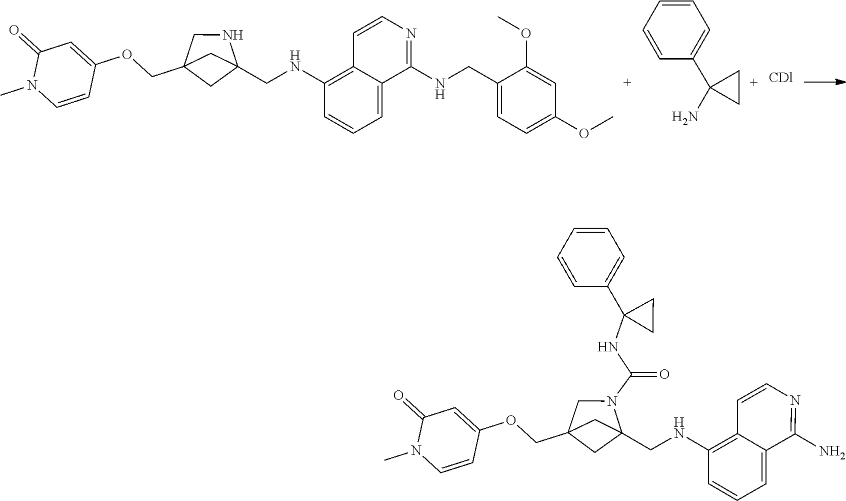

According to General Method 21a, 1-phenylcyclopropanamine (20 μL, 0.16 mmol) was reacted with CDI (25.8 mg, 0.151 mmol) and 4-((1-(((1-((2,4-dimethoxybenzyl)amino)isoquinolin-5-yl)amino)methyl)-2-azabicyclo[2.1.1]hexan-4-yl)methoxy)-1-methylpyridin-2(1H)-one (60 mg, 0.10 mmol). The mixture was concentrated to afford 1-(((1-((2,4-dimethoxybenzyl)amino)isoquinolin-5-yl)amino)methyl)-4-(((1-methyl-2-oxo-1,2-dihydropyridin-4-yl)oxy)methyl)-N-(1-phenylcyclopropyl)-2-azabicyclo[2.1.1]hexane-2-carboxamide which was deprotected according to General Method 8 in DCM (1 mL) at rt for 18 h. Flash chromatography (C18, 10-40% MeCN in 10 mM (NH₄)HCO₃ (aq)) afforded the product (33 mg, 61% yield).

[M+H]⁺=552.4

¹H NMR (DMSO-d6, 500 MHz) δ 1.13-1.22 (4H, m), 1.45-1.50 (2H, m), 1.78-1.83 (2H, m), 3.31 (3H, s), 3.36 (2H, s), 3.90 (2H, d, J=6.4 Hz), 4.15 (2H, s), 5.78 (1H, d, J=2.8 Hz), 5.89 (1H, dd, J=7.5, 2.7 Hz), 6.19 (1H, t, J=6.5 Hz), 6.55 (2H, s), 6.75 (1H, d, J=7.7 Hz), 6.79 (1H, d, J=6.2 Hz), 7.13-7.32 (8H, m), 7.53 (1H, d, J=7.6 Hz), 7.64 (1H, d, J=6.1 Hz)

### Example Number 59

### 1-(((1-Aminoisoquinolin-5-yl)amino)methyl)-4-(((5-oxo-1,2,3,5-tetrahydroindolizin-7-yl)oxy)methyl)-N-(1-phenylcyclopropyl)-2-azabicyclo[2.1.1]hexane-2-carboxamide

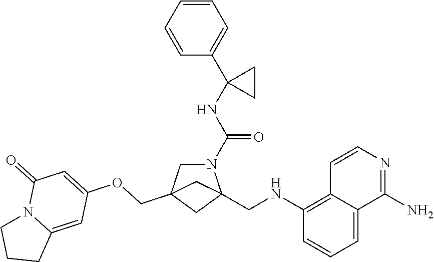 [SMILES: Nc1nccc2c(NCC34CC(COc5cc6n(c(=O)c5)CCC6)(CN3C(=O)NC3(c5ccccc5)CC3)C4)cccc12]

### 2-(tert-Butyl) 1-methyl 4-(((5-oxo-1,2,3,5-tetrahydroindolizin-7-yl)oxy)methyl)-2-azabicyclo[2.1.1]hexane-1,2-dicarboxylate

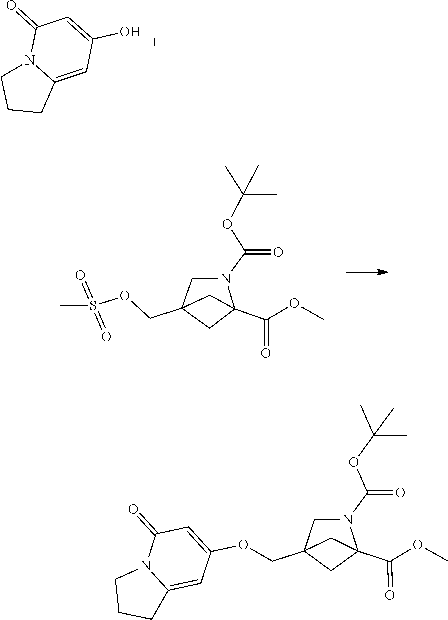

According to General Method 2b, 2-O-tert-butyl 1-O-methyl 4-(methylsulfonyloxymethyl)-2-azabicyclo[2.1.1]hexane-1,2-dicarboxylate (300 mg, 0.86 mmol) was reacted with 7-hydroxy-2,3-dihydro-1H-indolizin-5-one (143 mg, 0.94 mmol) in DMF (3 mL) using K₂CO₃ (356 mg, 2.58 mmol) at 60 °C for 20 h. Aqueous work up afforded the product (356 mg, 95% yield).

[M+H]⁺=405.3

### tert-Butyl 1-(hydroxymethyl)-4-(((5-oxo-1,2,3,5-tetrahydroindolizin-7-yl)oxy)methyl)-2-azabicyclo[2.1.1]hexane-2-carboxylate

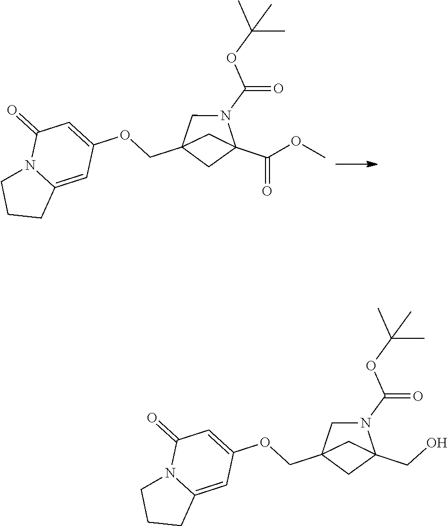 [SMILES: COC(=O)C12CC(COc3cc4n(c(=O)c3)CCC4)(CN1C(=O)OC(C)(C)C)C2 ; CC(C)(C)OC(=O)N1CC2(COc3cc4n(c(=O)c3)CCC4)CC1(CO)C2]

2-(tert-Butyl) 1-methyl 4-(((5-oxo-1,2,3,5-tetrahydroindolizin-7-yl)oxy)methyl)-2-azabicyclo[2.1.1]hexane-1,2-dicarboxylate (356 mg, 0.82 mmol) was reduced using LiBH₄ (2M in THF, 700 μL, 1.4 mmol) in anhydrous THF (5 mL) at rt for 4 days. Water (10 mL) and DCM (20 mL) were added. The organic layer was separated and the aqueous layer washed with DCM (2×10 mL). The combined organic layers were dried (MgSO₄), filtered and concentrated to afford the product (320 mg, 88% yield).

[M+H]⁺=377.2

### tert-Butyl 1-formyl-4-(((5-oxo-1,2,3,5-tetrahydroindolizin-7-yl)oxy)methyl)-2-azabicyclo[2.1.1]hexane-2-carboxylate

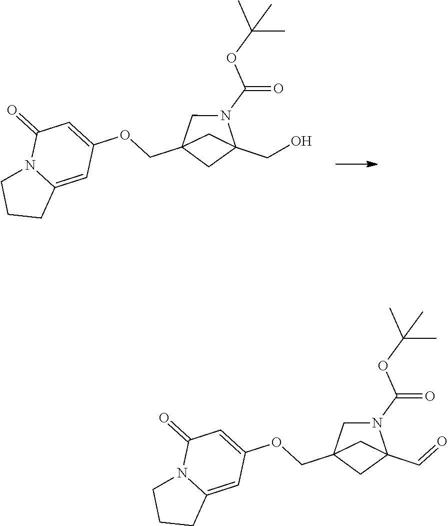 [SMILES: CC(C)(C)OC(=O)N1CC2(COc3cc4n(c(=O)c3)CCC4)CC1(CO)C2 ; CC(C)(C)OC(=O)N1CC2(COc3cc4n(c(=O)c3)CCC4)CC1(C=O)C2]

According to General Method 16, tert-butyl 1-(hydroxymethyl)-4-[(5-oxo-2,3-dihydro-1H-indolizin-7-yl)oxymethyl]-2-azabicyclo[2.1.1]hexane-2-carboxylate (376 mg, 0.85 mmol) was oxidised over 48 h. Aqueous work up afforded the product (271 mg, 85% yield).

[M+H]⁺=375.2

### tert-Butyl 1-(((1-((2,4-dimethoxybenzyl)amino)isoquinolin-5-yl)amino)methyl)-4-(((5-oxo-1,2,3,5-tetrahydroindolizin-7-yl)oxy)methyl)-2-azabicyclo[2.1.1]hexane-2-carboxylate

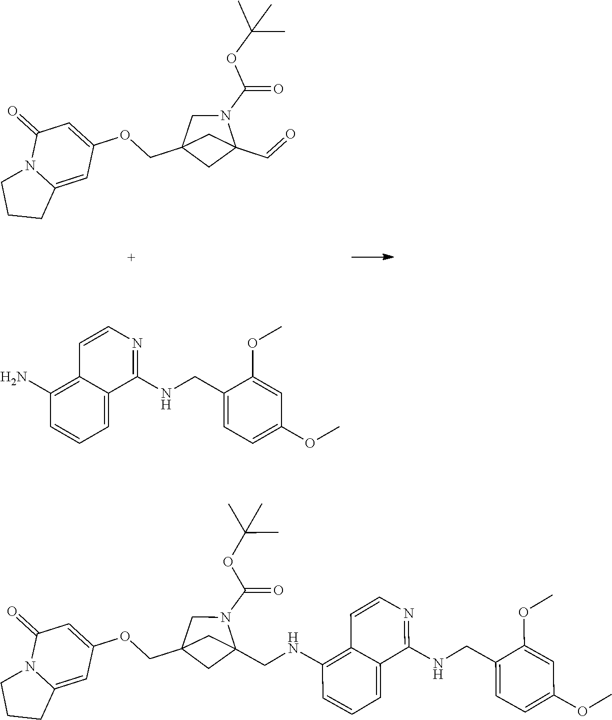 [SMILES: CC(C)(C)OC(=O)N1CC2(COc3cc4n(c(=O)c3)CCC4)CC1(C=O)C2 ; COc1ccc(CNc2nccc3c(N)cccc23)c(OC)c1 ; COc1ccc(CNc2nccc3c(NCC45CC(COc6cc7n(c(=O)c6)CCC7)(CN4C(=O)OC(C)(C)C)C5)cccc23)c(OC)c1]

According to General Method 17, tert-butyl 1-formyl-4-[(5-oxo-2,3-dihydro-1H-indolizin-7-yl)oxymethyl]-2-azabicyclo[2.1.1]hexane-2-carboxylate (271 mg, 0.72 mmol) was reacted with 1-N-[(2,4-dimethoxyphenyl)methyl]isoquinoline-1,5-diamine (335 mg, 1.08 mmol) for 20 h. Aqueous work up and purification by flash chromatography (Silica, 0-100% EtOAc in Pet. Ether, followed by 0-20% MeOH in EtOAc) afforded the product (403 mg, 84% yield).

[M+H]⁺=668.4

### 7-((1-(((1-Aminoisoquinolin-5-yl)amino)methyl)-2-azabicyclo[2.1.1]hexan-4-yl)methoxy)-2,3-dihydroindolizin-5(1H)-one

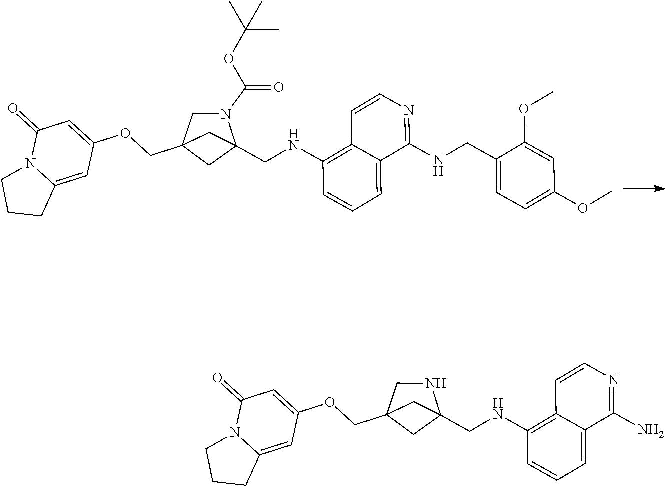 [SMILES: COc1ccc(CNc2nccc3c(NCC45CC(COc6cc7n(c(=O)c6)CCC7)(CN4C(=O)OC(C)(C)C)C5)cccc23)c(OC)c1 ; Nc1nccc2c(NCC34CC(COc5cc6n(c(=O)c5)CCC6)(CN3)C4)cccc12]

tert-Butyl 1-(((1-((2,4-dimethoxybenzyl)amino)isoquinolin-5-yl)amino)methyl)-4-(((5-oxo-1,2,3,5-tetrahydroindolizin-7-yl)oxy)methyl)-2-azabicyclo[2.1.1]hexane-2-carboxylate (100 mg, 0.15 mmol) was deprotected according to General Method 8 at rt for 60 min to afford the product (55 mg, 70% yield).

[M+H]⁺=418.2

### N-(1-Phenylcyclopropyl)-1H-imidazole-1-carboxamide

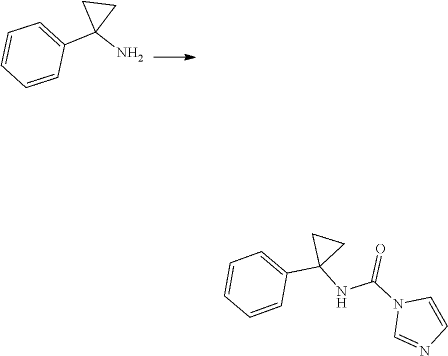 [SMILES: NC1(c2ccccc2)CC1 ; O=C(NC1(c2ccccc2)CC1)n1ccnc1]

To a solution of 1-phenylcyclopropan-1-amine (500 mg, 3.75 mmol) and DBU (95 μL, 0.64 mmol) in dry DCM (10 mL) was added CDI (621 mg, 3.83 mmol) and stirred at rt for 1 h. The mixture was quenched with water (50 mL), extracted with DCM (2×50 mL), washed with brine (50 mL), dried (MgSO₄) and concentrated to afford the product (570 mg, 53% yield).

[M+H]⁺=228.1

### 1-(((1-Aminoisoquinolin-5-yl)amino)methyl)-4-(((5-oxo-1,2,3,5-tetrahydroindolizin-7-yl)oxy)methyl)-N-(1-phenylcyclopropyl)-2-azabicyclo[2.1.1]hexane-2-carboxamide

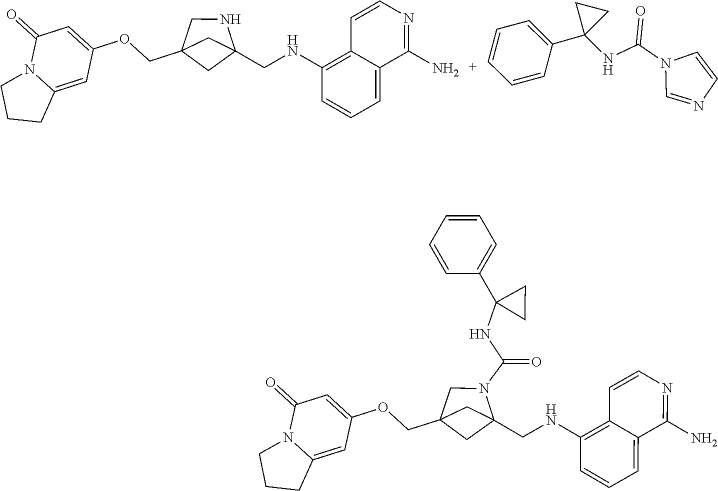 [SMILES: Nc1nccc2c(NCC34CC(COc5cc6n(c(=O)c5)CCC6)(CN3)C4)cccc12 ; O=C(NC1(c2ccccc2)CC1)n1ccnc1 ; Nc1nccc2c(NCC34CC(COc5cc6n(c(=O)c5)CCC6)(CN3C(=O)NC3(c5ccccc5)CC3)C4)cccc12]

To a solution of N-(1-phenylcyclopropyl)imidazole-1-carboxamide (45 mg, 0.2 mmol) and 7-[[1-[[(1-aminoisoquinolin-5-yl)amino]methyl]-2-azabicyclo[2.1.1]hexan-4-yl]methoxy]-2,3-dihydro-1H-indolizin-5-one (70 mg, 0.13 mmol) in dry DMF (1.5 mL) was added DBU (30 μL, 0.2 mmol) after 10 min. The reaction was stirred at rt for 18 h, quenched with water (10 mL) and concentrated. The residue was partitioned between EtOAc (10 mL) and water (10 mL). The aqueous layer was re-extracted with EtOAc (3×20 mL) and the combined organics were dried (MgSO₄) and concentrated. Purification by automated HPLC (mass directed 2-60% MeCN in [2.5% NH₃ in water]) afforded the product (24 mg, 32% yield).

[M+H]⁺=577.3

¹H NMR (DMSO-d6, 400 MHz) δ 1.12-1.24 (4H, m), 1.41-1.53 (2H, m), 1.76-1.84 (2H, m), 1.97-2.10 (2H, m), 2.95 (2H, t, J=7.7 Hz), 3.36 (2H, s), 3.79-3.86 (2H, m), 3.90 (2H, d, J=6.4 Hz), 4.13 (2H, s), 5.61 (1H, d, J=2.5 Hz), 5.80-5.89 (1H, m), 6.18 (1H, t, J=6.5 Hz), 6.51 (2H, s), 6.73 (1H, d, J=7.7 Hz), 6.78 (1H, d, J=6.0 Hz), 7.13-7.22 (4H, m), 7.23-7.32 (4H, m), 7.64 (1H, d, J=6.1 Hz)

### Example Number 65

### 1-(((1-Aminoisoquinolin-5-yl)amino)methyl)-N-(2,3-dihydro-1H-inden-5-yl)-4-(((1,6-dimethyl-2-oxo-1,2-dihydropyridin-4-yl)oxy)methyl)-2-azabicyclo[2.1.1]hexane-2-carboxamide

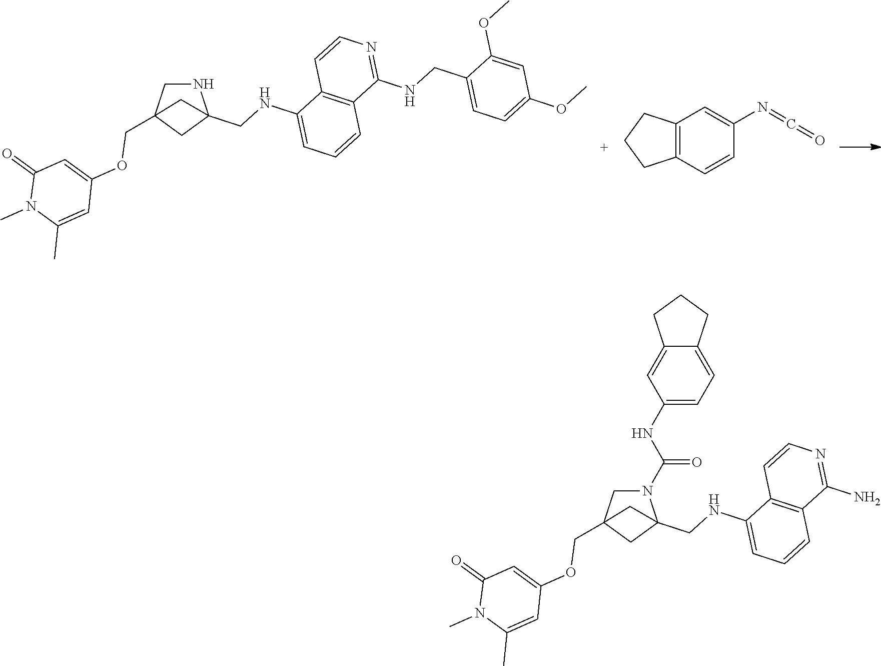

According to General Method 21c, 5-indanyl isocyanate (30 mg, 0.18 mmol) was reacted with 4-((1-(((1-((2,4-dimethoxybenzyl)amino)isoquinolin-5-yl)amino)methyl)-2-azabicyclo[2.1.1]hexan-4-yl)methoxy)-1,6-dimethylpyridin-2(1H)-one (77 mg, 0.13 mmol) for 2 h. Concentration afforded the intermediate N-(2,3-dihydro-1H-inden-5-yl)-1-(((1-((2,4-dimethoxybenzyl)amino)isoquinolin-5-yl)amino)methyl)-4-(((1,6-dimethyl-2-oxo-1,2-dihydropyridin-4-yl)oxy)methyl)-2-azabicyclo[2.1.1]hexane-2-carboxamide which was deprotected according to General Method 8, using TFA (250 μL, 3.33 mmol) in DCM (2 mL) at rt for 18 h. Following work up (iv), purification by flash chromatography (C18, 20-52% MeCN in 10 mM (NH₄)HCO₃ (aq)) afforded the product (23 mg, 32%).

[M+H]⁺=565.7

1H NMR (DMSO-d6, 400 MHz) δ 1.52-1.58 (2H, m), 1.84 (2H, d, J=4.6 Hz), 1.99 (2H, p, J=7.4 Hz), 2.26 (3H, s), 2.80 (4H, dt, J=11.3, 7.4 Hz), 3.30 (3H, s), 3.50 (2H, s), 3.99 (2H, d, J=6.2 Hz), 4.14 (2H, s), 5.69 (1H, d, J=2.8 Hz), 5.84 (1H, dd, J=2.8, 0.9 Hz), 6.20 (1H, t, J=6.4 Hz), 6.50 (2H, s), 6.74 (1H, d, J=7.7 Hz), 6.98 (1H, d, J=6.2 Hz), 7.07 (1H, d, J=8.1 Hz), 7.17-7.26 (2H, m), 7.33 (1H, d, J=8.3 Hz), 7.41 (1H, s), 7.71 (1H, d, J=6.0 Hz), 8.40 (1H, s)

### Example Number 66

### 1-(((1-Aminoisoquinolin-5-yl)amino)methyl)-4-(((1-methyl-2-oxo-1,2-dihydropyridin-4-yl)oxy)methyl)-N-(2-phenylpropan-2-yl)-2-azabicyclo[2.1.1]hexane-2-carboxamide

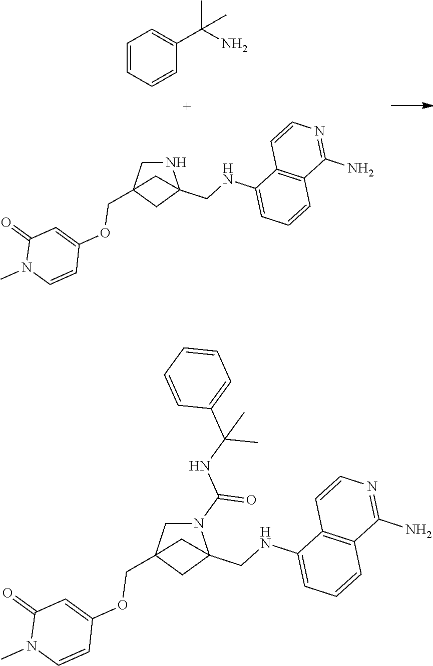

According to General Method 21b, 2-phenylpropan-2-amine (24 mg, 0.25 mmol) was reacted for 18 h and a further 3 h following the addition of 4-((1-(((1-aminoisoquinolin-5-yl)amino)methyl)-2-azabicyclo[2.1.1]hexan-4-yl)methoxy)-1-methylpyridin-2(1H)-one (53 mg, 0.13 mmol). Concentration and purification by flash chromatography (C18, 20-47% MeCN in 10 mM (NH₄)HCO₃ (aq)) followed by automated prep HPLC (UV directed 31-41% MeCN in 10 mM (NH₄)HCO₃ (aq)) over 8 min) afforded the product (70 mg, 94% yield).

[M+H]⁺=553.7

¹H NMR (DMSO-d6, 500 MHz) δ 1.44-1.53 (2H, m), 1.56 (6H, s), 1.76-1.81 (2H, m), 3.31 (3H, s), 3.46 (2H, s), 3.81 (2H, d, J=6.3 Hz), 4.16 (2H, s), 5.79 (1H, d, J=2.8 Hz), 5.91 (1H, dd, J=7.5, 2.7 Hz), 6.04 (1H, t, J=6.4 Hz), 6.42 (1H, s), 6.47 (2H, s), 6.63-6.72 (2H, m), 7.13-7.22 (2H, m), 7.25-7.32 (3H, m), 7.35-7.41 (2H, m), 7.54 (1H, d, J=7.6 Hz), 7.64 (1H, d, J=6.1 Hz)

### Example Number 71

### 4-(((1,6-Dimethyl-2-oxo-1,2-dihydropyridin-4-yl)oxy)methyl)-1-(((2-hydroxypyridin-3-yl)amino)methyl)-N-(1-phenylcyclopropyl)-2-azabicyclo[2.1.1]hexane-2-carboxamide

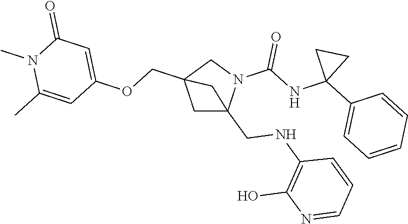 [SMILES: Cc1cc(OCC23CN(C(=O)NC4(c5ccccc5)CC4)C(CNc4cccnc4O)(C2)C3)cc(=O)n1C]

### tert-Butyl 4-(((1,6-dimethyl-2-oxo-1,2-dihydropyridin-4-yl)oxy)methyl)-1-(((2-methoxypyridin-3-yl)amino)methyl)-2-azabicyclo[2.1.1]hexane-2-carboxylate

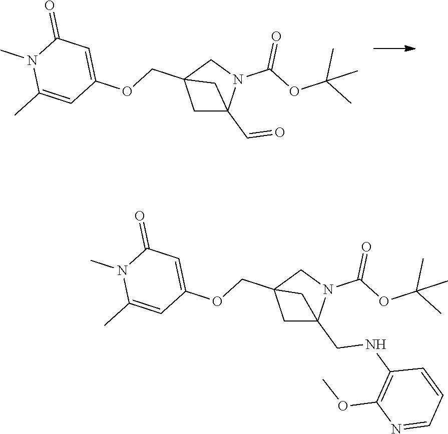

According to General Method 17, tert-butyl 4-[(1,2-dimethyl-6-oxopyridin-4-yl)oxymethyl]-1-formyl-2-azabicyclo[2.1.1]hexane-2-carboxylate (100 mg, 0.28 mmol) was reacted with 3-amino-2-methoxypyridine (38 mg, 0.3 mmol) at rt for 18 h. Aqueous work up and purification by HPLC (mass directed 2-60% MeCN in [2.5% NH₄OH in water]) afforded the product (53 mg, 41% yield).

[M+H]⁺=471.3

### 4-[[1-[[(2-Hydroxypyridin-3-yl)amino]methyl]-2-azabicyclo[2.1.1]hexan-4-yl]methoxy]-1,6-dimethylpyridin-2-one hydrochloride

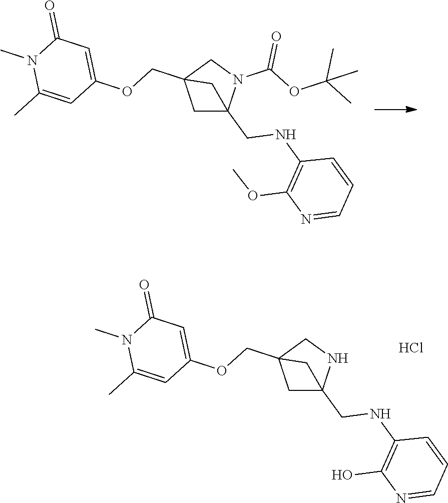

According to General Method 5a, tert-butyl 4-(((1,6-dimethyl-2-oxo-1,2-dihydropyridin-4-yl)oxy)methyl)-1-(((2-methoxypyridin-3-yl)amino)methyl)-2-azabicyclo[2.1.1]hexane-2-carboxylate (54 mg, 0.11 mmol) was deprotected at rt for 3 days. The solution was concentrated to afford the product (44 mg, 98% yield).

[M+H]⁺=357.2

### 4-(((1,6-Dimethyl-2-oxo-1,2-dihydropyridin-4-yl)oxy)methyl)-1-(((2-hydroxypyridin-3-yl)amino)methyl)-N-(1-phenylcyclopropyl)-2-azabicyclo[2.1.1]hexane-2-carboxamide

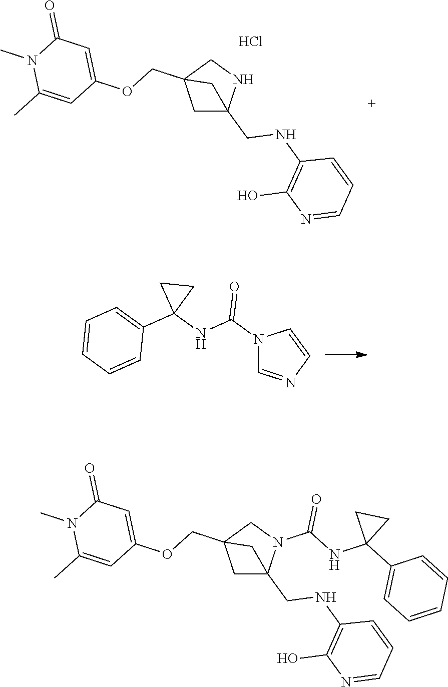 [SMILES: Cc1cc(OCC23CNC(CNc4cccnc4O)(C2)C3)cc(=O)n1C ; Cl ; O=C(NC1(c2ccccc2)CC1)n1ccnc1 ; Cc1cc(OCC23CN(C(=O)NC4(c5ccccc5)CC4)C(CNc4cccnc4O)(C2)C3)cc(=O)n1C]

To a solution of 4-[[1-[[(2-hydroxypyridin-3-yl)amino]methyl]-2-azabicyclo[2.1.1]hexan-4-yl]methoxy]-1,6-dimethylpyridin-2-one hydrochloride (43 mg, 0.11 mmol) in MeCN (2 mL) and DMF (1 mL) was added DBU (33 μL, 0.22 mmol). After 10 min N-(1-phenylcyclopropyl)imidazole-1-carboxamide (64 mg, 0.22 mmol) was added and the reaction stirred at rt for 36 h. The reaction mixture was quenched with water (10 mL) and extracted with EtOAc (3×20 mL) before being washed with brine (2×10 mL), dried (MgSO₄) and concentrated. Purification by HPLC (mass directed 2-60% MeCN in [2.5% NH₄OH in water]) afforded the product (21 mg, 37% yield).

[M+H]⁺=516.3

1H NMR (DMSO-d6, 400 MHz) δ 1.05-1.23 (4H, m), 1.39-1.53 (2H, m), 1.75 (2H, d, J=4.3 Hz), 2.28 (3H, s), 3.32 (3H, s), 3.35 (2H, s), 3.73 (2H, d, J=6.5 Hz), 4.15 (2H, s), 5.50 (1H, t, J=6.7 Hz), 5.72 (1H, d, J=2.8 Hz), 5.89 (1H, d, J=2.5 Hz), 6.02 (1H, t, J=6.8 Hz), 6.24 (1H, dd, J=7.2, 1.6 Hz), 6.49-6.60 (1H, m), 7.06-7.18 (3H, m), 7.19-7.30 (3H, m), 11.32 (1H, s)

### Example Number 90

### 1-(((1-Aminoisoquinolin-5-yl)amino)methyl)-N-(2,3-dihydrobenzofuran-3-yl)-4-(((1,6-dimethyl-2-oxo-1,2-dihydropyridin-4-yl)oxy)methyl)-N-methyl-2-azabicyclo[2.1.1]hexane-2-carboxamide

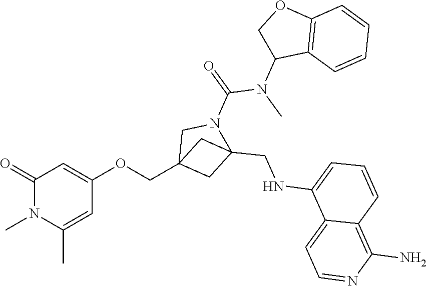 [SMILES: Cc1cc(OCC23CN(C(=O)N(C)C4COc5ccccc54)C(CNc4cccc5c(N)nccc45)(C2)C3)cc(=O)n1C]

### tert-Butyl N-(2,3-dihydrobenzofuran-3-yl)carbamate

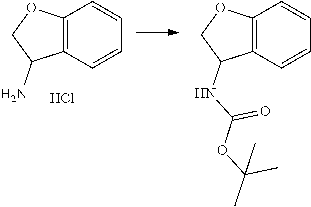 [SMILES: NC1COc2ccccc21 ; Cl ; CC(C)(C)OC(=O)NC1COc2ccccc21]

Boc₂O (766 μL, 3.33 mmol) was added to a mixture of 2,3-dihydrobenzofuran-3-amine hydrochloride (500 mg, 2.91 mmol) and TEA (1.3 mL, 9.33 mmol) in DCM (10 mL) at rt and stirred for 18 h. The mixture was quenched with brine (20 mL) and DCM (20 mL). The layers were separated, and the organic layer was washed with brine (20 mL), dried (Na₂SO₄), filtered, and concentrated to afford the product (648 mg, 85% yield).

¹H NMR (CDCl₃, 400 MHz) δ 1.46 (9H, s), 4.33 (1H, dd, J=10.0, 4.1 Hz), 4.65 (1H, dd, J=10.0, 7.8 Hz), 4.88 (1H, s), 5.35 (1H, s), 6.81-6.85 (1H, m), 6.91 (1H, td, J=7.5, 1.0 Hz), 7.22 (1H, dddd, J=8.0, 7.4, 1.4, 0.6 Hz), 7.33 (1H, d, J=7.4 Hz)

### tert-Butyl (2,3-dihydrobenzofuran-3-yl)(methyl)carbamate

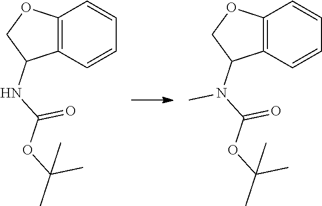 [SMILES: CC(C)(C)OC(=O)NC1COc2ccccc21 ; CN(C(=O)OC(C)(C)C)C1COc2ccccc21]

According to General Method 2a, tert-butyl N-(2,3-dihydrobenzofuran-3-yl)carbamate (648 mg, 2.48 mmol) was reacted with Mel (231 μL, 3.72 mmol) at rt for 18 h. The mixture was quenched with brine (20 mL) and extracted with EtOAc (3×20 mL). The combined organic layers were washed with brine (20 mL), dried (Na₂SO₄), filtered, and concentrated to afford the product.

### N-Methyl-2,3-dihydrobenzofuran-3-amine hydrochloride

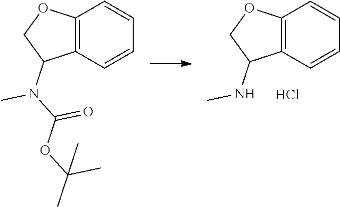 [SMILES: CN(C(=O)OC(C)(C)C)C1COc2ccccc21 ; CNC1COc2ccccc21 ; Cl]

According to a variation on General Method 5a, tert-butyl (2,3-dihydrobenzofuran-3-yl)(methyl)carbamate was deprotected in MeOH (5 mL) for 96 h at rt. The mixture was concentrated. THF (3 mL) was added, and the mixture was sonicated for 1 min, filtered, and washed with THF (2×5 mL), hexanes (2×5 mL), and Et₂O (2×5 mL) to afford the product (501 mg, 44% yield).

¹H NMR (D₂O, 400 MHz) δ 2.72 (3H, s), 4.72 (1H, dd, J=12.0, 7.3 Hz), 4.84 (1H, d, J=12.0 Hz), 5.09 (1H, d, J=7.2 Hz), 7.06 (1H, d, J=8.1 Hz), 7.14 (1H, t, J=7.6 Hz), 7.49 (1H, t, J=7.9 Hz), 7.61 (1H, d, J=7.6 Hz)

### (2,3-Dihydrobenzofuran-3-yl)(methyl)carbamic chloride

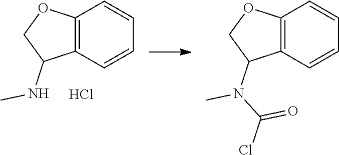

According to a modification of General Method 21b Part 1, N-methyl-2,3-dihydrobenzofuran-3-amine HCl (100 mg, 0.27 mmol, 40% pure) was reacted in anhydrous DCM (1 mL) at 0 °C for 30 min then at rt for 18 h. After quenching with 1M HCl (aq) (15 mL), the aqueous phase was extracted with DCM (3×25 mL). The combined organic layers were washed with 1M HCl (aq) (25 mL), sat. NaHCO₃ (aq) (25 mL), dried (Na₂SO₄), filtered, and concentrated. The product was used directly in the next step.

¹H NMR (CDCl₃, 500 MHz) δ 0.32 (4H, s), 1.25 (1H, d, J=6.5 Hz), 1.36-1.39 (3H, m), 3.25-3.28 (4H, m)

### 1-(((1-Aminoisoquinolin-5-yl)amino)methyl)-N-(2,3-dihydrobenzofuran-3-yl)-4-(((1,6-dimethyl-2-oxo-1,2-dihydropyridin-4-yl)oxy)methyl)-N-methyl-2-azabicyclo[2.1.1]hexane-2-carboxamide

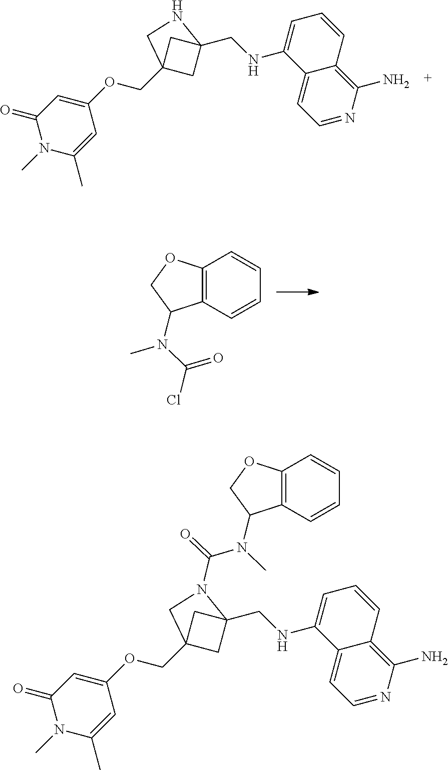 [SMILES: Cc1cc(OCC23CNC(CNc4cccc5c(N)nccc45)(C2)C3)cc(=O)n1C ; CN(C(=O)Cl)C1COc2ccccc21 ; Cc1cc(OCC23CN(C(=O)N(C)C4COc5ccccc54)C(CNc4cccc5c(N)nccc45)(C2)C3)cc(=O)n1C]

According to a modification of General Method 21b, part 2, 4-[[1-[[(1-amino-5-isoquinolyl)amino]methyl]-2-azabicyclo[2.1.1]hexan-4-yl]methoxy]-1,6-dimethylpyridin-2-one (50 mg, 0.12 mmol, 95% purity) was reacted with N-(2,3-dihydrobenzofuran-3-yl)-N-methyl-carbamoyl chloride (61 mg, 0.14 mmol) in THF (2.00 mL) at rt for 72 h. The mixture was concentrated. Purification by flash chromatography (C18, 25-56% MeCN in 10 mM (NH₄)HCO₃ (aq)) followed by automated prep HPLC (UV directed 38-48% MeCN in 10 mM (NH₄)HCO₃ (aq) over 8 min) afforded the product (19 mg, 28% yield).

[M+H]⁺=581.4

¹H NMR (DMSO-d6, 400 MHz) δ 1.59 (2H, t, J=4.2 Hz), 1.77 (2H, t, J=6.2 Hz), 2.25 (3H, s), 2.52-2.55 (3H, m), 3.28-3.30 (4H, m), 3.38 (1H, d, J=7.3 Hz), 3.83 (1H, dd, J=14.5, 6.2 Hz), 3.92 (1H, dd, J=14.5, 6.0 Hz), 4.10 (2H, s), 4.45 (1H, dd, J=10.3, 4.2 Hz), 4.65 (1H, dd, J=10.2, 9.2 Hz), 5.66 (1H, d, J=2.8 Hz), 5.83 (1H, d, J=2.6 Hz), 5.97 (1H, dd, J=9.1, 4.2 Hz), 6.17 (1H, t, J=6.2 Hz), 6.48 (2H, s), 6.75 (1H, d, J=7.8 Hz), 6.87 (1H, d, J=8.0 Hz), 6.92 (1H, ddd, J=8.3, 7.0, 0.9 Hz), 7.10 (1H, d, J=6.2 Hz), 7.20 (1H, t, J=8.0 Hz), 7.22-7.29 (2H, m), 7.30 (1H, d, J=8.6 Hz), 7.71 (1H, d, J=6.1 Hz)

### Example Number 63

### 1-(((1-Aminoisoquinolin-5-yl)amino)methyl)-4-(((1-methyl-2-oxo-1,2-dihydropyridin-4-yl)oxy)methyl)-2-azabicyclo[2.1.1]hexane-2-carboxamide

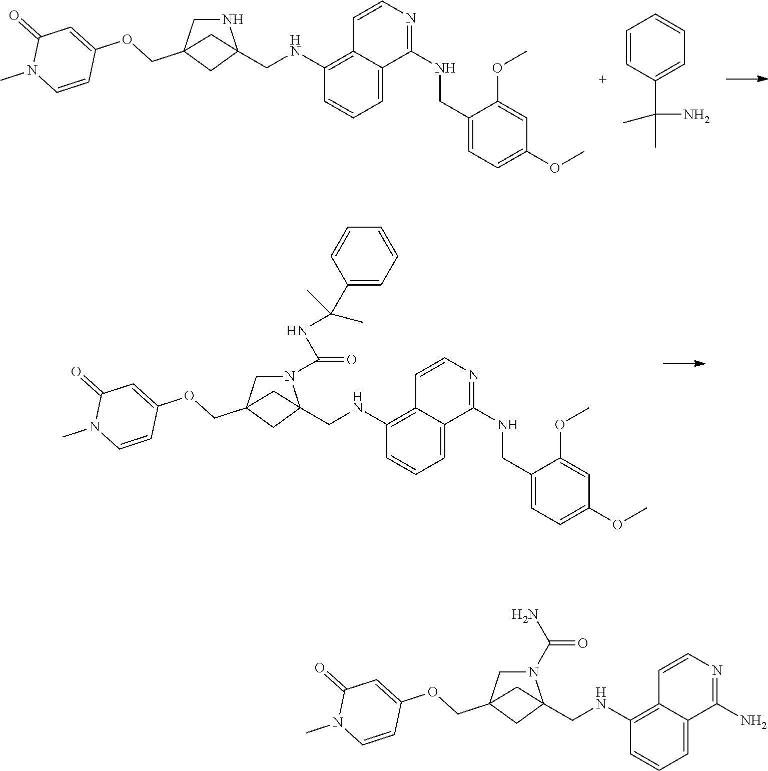

According to General Method 21b, cumylamine (30 mg, 0.22 mmol) was reacted in DCM (1.5 mL) at rt for 90 min and then with 4-((1-(((1-((2,4-dimethoxybenzyl)amino)isoquinolin-5-yl)amino)methyl)-2-azabicyclo[2.1.1]hexan-4-yl)methoxy)-1-methylpyridin-2(1H)-one (81 mg, 0.142 mmol) for 36 h. Aqueous work up afforded 1-(((1-((2,4-dimethoxybenzyl)amino)isoquinolin-5-yl)amino)methyl)-4-(((1-methyl-2-oxo-1,2-dihydropyridin-4-yl)oxy)methyl)-N-(2-phenylpropan-2-yl)-2-azabicyclo[2.1.1]hexane-2-carboxamide which was deprotected according to General Method 8 in DCM (2 mL) and TFA (250 μL, 3.33 mmol) at rt for 18 h. Purification by flash chromatography (C18, 20-32% MeCN in 10 mM (NH₄)HCO₃ (aq)) followed by automated prep HPLC (UV directed 14-24% MeCN in 10 mM (NH₄)HCO₃ (aq) over 8 min) afforded the product (12.8 mg, 13% yield).

[M+H]⁺=435.6

¹H NMR (DMSO-d6, 400 MHz) δ 1.45-1.52 (2H, m), 1.77-1.85 (2H, m), 3.30 (2H, s), 3.30 (3H, s), 3.89 (2H, d, J=6.2 Hz), 4.13 (2H, s), 5.77 (1H, d, J=2.7 Hz), 5.89 (1H, dd, J=7.5, 2.7 Hz), 5.92 (2H, s), 6.50-6.59 (3H, m), 6.77 (1H, d, J=7.7 Hz), 6.88 (1H, d, J=6.2 Hz), 7.20 (1H, t, J=8.0 Hz), 7.31 (1H, d, J=8.3 Hz), 7.53 (1H, d, J=7.5 Hz), 7.70 (1H, d, J=6.1 Hz)

### Example Number 91

### 1-(((1-Aminoisoquinolin-5-yl)amino)methyl)-4-(((1-methyl-2-oxo-1,2-dihydropyridin-4-yl)oxy)methyl)-N-phenyl-2-azabicyclo[2.1.1]hexane-2-carbothioamide

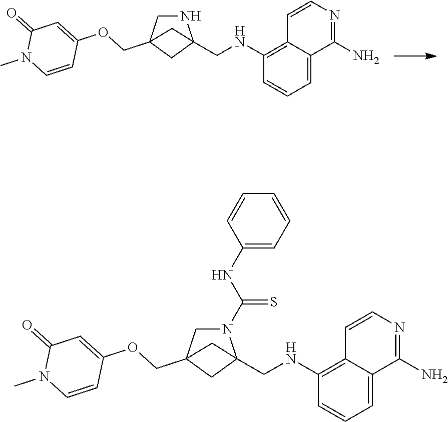 [SMILES: Cn1ccc(OCC23CNC(CNc4cccc5c(N)nccc45)(C2)C3)cc1=O ; Cn1ccc(OCC23CN(C(=S)Nc4ccccc4)C(CNc4cccc5c(N)nccc45)(C2)C3)cc1=O]

A solution of 4-[[1-[[(1-amino-5-isoquinolyl)amino]methyl]-2-azabicyclo[2.1.1]hexan-4-yl]methoxy]-1-methylpyridin-2-one (18 mg, 0.05 mmol) and TEA (20 μL, 0.14 mmol) in DMSO (750 μL) was added to isothiocyanatobenzene (8 mg, 0.06 mmol), and the mixture was stirred for 16 h. The mixture was quenched with MeOH (250 μL), filtered and purified by HPLC (mass directed, MeCN in 10 mM (NH₄)HCO₃ (aq)) to provide the product (16 mg, 64% yield).

[M+H]⁺=527.1

¹H NMR (DMSO-d6, 300 MHz) δ 1.90 (2H, s), 2.04 (2H, s), 3.32 (3H, s), 3.74 (2H, s), 4.07 (2H, s), 4.23 (2H, s), 5.83 (1H, d, J=2.6 Hz), 5.93 (1H, dd, J=7.5, 2.7 Hz), 6.20 (1H, br s), 6.59 (2H, s), 6.83-6.97 (2H, m), 7.05-7.20 (5H, m), 7.24 (1H, t, J=8.0 Hz), 7.49 (1H, d, J=8.6 Hz), 7.56 (1H, d, J=7.6 Hz), 7.79 (1H, d, J=6.0 Hz), 9.93 (1H, s)

### Example Number 95

### 4-((1-(((1-Aminoisoquinolin-5-yl)amino)methyl)-2-(6-cyclopropylpyridazin-3-yl)-2-azabicyclo[2.1.1]hexan-4-yl)methoxy)-1,6-dimethylpyridin-2(1H)-one

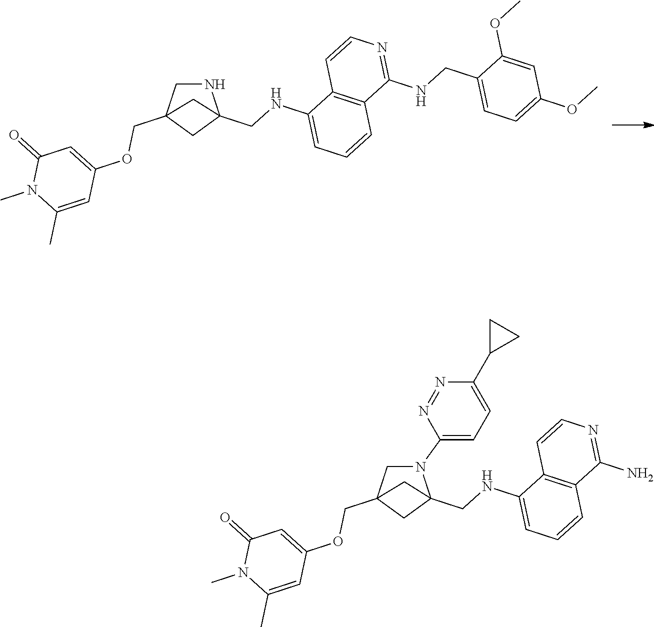

According to General Method 4, 4-((1-(((1-((2,4-dimethoxybenzyl)amino)isoquinolin-5-yl)amino)methyl)-2-azabicyclo[2.1.1]hexan-4-yl)methoxy)-1-methylpyridin-2(1H)-one (60 mg, 0.11 mmol) was reacted with 3-chloro-6-cyclopropyl-pyridazine (20 mg, 0.129 mmol), using NaO^{t}Bu (16 mg, 0.16 mmol), RuPhosPd-G3 (13 mg, 0.0148 mmol) and RuPhos (7 mg, 0.0147 mmol) in THF (1 mL) at reflux for 16 h. The mixture was cooled to rt, diluted with MeOH (500 μL), filtered, washing with MeOH (5 mL) and the filtrate was concentrated to afford the intermediate 4-((2-(6-cyclopropylpyridazin-3-yl)-1-(((1-((2,4-dimethoxybenzyl)amino)isoquinolin-5-yl)amino)methyl)-2-azabicyclo[2.1.1]hexan-4-yl)methoxy)-1,6-dimethylpyridin-2(1H)-one which was deprotected according to General Method 8, in DCM (2 mL) at rt for 4 h. Aqueous work up and purification by flash chromatography (C18, 20-50% MeCN in 10 mM (NH₄)HCO₃ (aq)) afforded the product (32 mg, 57% yield).

[M+H]⁺=524.4

¹H NMR (DMSO-d6, 300 MHz) δ 0.88-1.02 (4H, m), 1.66 (2H, d, J=3.3 Hz), 1.91 (2H, d, J=4.2 Hz), 2.05-2.16 (1H, m), 2.25 (3H, s), 3.30 (3H, s), 3.42 (2H, s), 4.14 (2H, s), 4.18 (2H, d, J=6.1 Hz), 5.68 (1H, d, J=2.6 Hz), 5.83 (1H, d, J=2.6 Hz), 6.43-6.55 (3H, m), 6.76 (1H, d, J=7.6 Hz), 6.96 (1H, d, J=9.2 Hz), 7.01 (1H, d, J=6.1 Hz), 7.17-7.29 (2H, m), 7.32 (1H, d, J=8.4 Hz), 7.69 (1H, d, J=6.1 Hz)

### Example Number 97

### 4-((1-(((1-Aminoisoquinolin-5-yl)amino)methyl)-2-(4,5-dihydrooxazol-2-yl)-2-azabicyclo[2.1.1]hexan-4-yl)methoxy)-1,6-dimethylpyridin-2(1H)-one

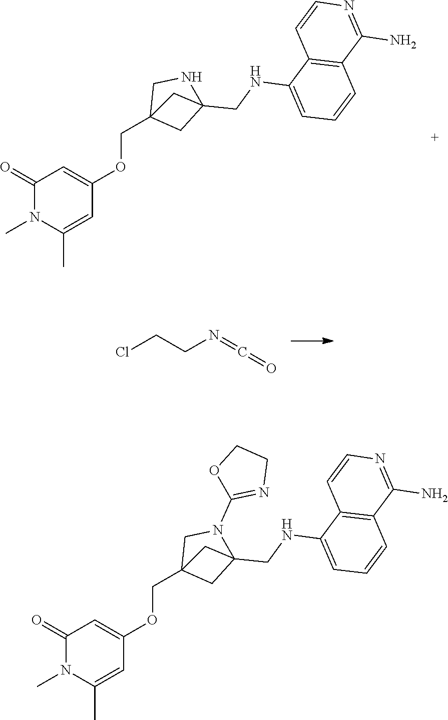 [SMILES: Cc1cc(OCC23CNC(CNc4cccc5c(N)nccc45)(C2)C3)cc(=O)n1C ; O=C=NCCCl ; Cc1cc(OCC23CN(C4=NCCO4)C(CNc4cccc5c(N)nccc45)(C2)C3)cc(=O)n1C]

According to General Method 21c, 4-((1-(((1-aminoisoquinolin-5-yl)amino)methyl)-2-azabicyclo[2.1.1]hexan-4-yl)methoxy)-1,6-dimethylpyridin-2(1H)-one (100 mg, 0.25 mmol) was reacted with 1-chloro-2-isocyanato-ethane (31 μL, 0.35 mmol) in anhydrous THF (5 mL) at 80 °C for 42 h. Concentration and purification by flash chromatography (C18, 20-51% MeCN in 10 mM NH₄HCO₃ (aq)) followed by automated prep HPLC (UV directed 30-40% MeCN in (10 mM NH₄HCO₃ (aq)) over 8 min) afforded the product (4 mg, 4% yield).

[M+H]⁺=475.4

¹H NMR (DMSO-d6, 400 MHz) δ 1.55 (2H, s), 1.86 (2H, d, J=4.7 Hz), 2.29 (3H, s), 3.32 (3H, s), 3.37 (2H, s), 3.79 (2H, t, J=8.7 Hz), 3.95 (2H, d, J=5.8 Hz), 4.14 (2H, s), 4.27 (2H, t, J=8.7 Hz), 5.70 (1H, d, J=2.8 Hz), 5.87 (1H, d, J=2.8 Hz), 6.52 (2H, d, J=4.2 Hz), 6.69-6.79 (2H, m), 7.00 (1H, d, J=6.2 Hz), 7.23 (1H, t, J=8.0 Hz), 7.34 (1H, d, J=8.3 Hz), 7.75 (1H, d, J=6.1 Hz)

### Example Number 102

### 4-((4-(((1-Aminoisoquinolin-5-yl)amino)methyl)-2-(3-cyclohexylpropanoyl)-2-azabicyclo[2.1.1]hexan-1-yl)methoxy)-1-methylpyridin-2(1H)-one

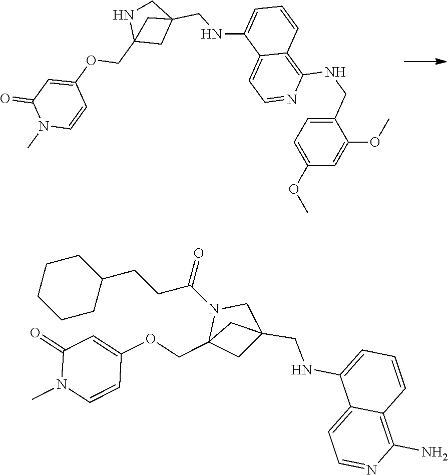

According to General Method 18a, 3-cyclohexylpropanoic acid (25 mg, 0.16 mmol) was coupled to 4-((1-(((1-((2,4-dimethoxybenzyl)amino)isoquinolin-5-yl)amino)methyl)-2-azabicyclo[2.1.1]hexan-4-yl)methoxy)-1-methylpyridin-2(1H)-one (80 mg, 0.15 mmol) over 18 h. The mixture was concentrated to afford 4-((2-(3-cyclohexylpropanoyl)-1-(((1-((2,4-dimethoxybenzyl)amino)isoquinolin-5-yl)amino)methyl)-2-azabicyclo[2.1.1]hexan-4-yl)methoxy)-1-methylpyridin-2(1H)-one which was deprotected according to General Method 8 in DCM (1 mL) and TFA (1379 μL, 13.5 mmol) at rt for 24 h. Purification by flash chromatography (C18, 5-60% MeCN in 10 mM (NH₄)HCO₃ (aq)) provided the product (38 mg, 48% yield).

[M+H]⁺=530.3

¹H NMR (400 MHz, DMSO-d6) δ 0.86 (2H t, J=11.7 Hz), 1.06-1.26 (5H, m), 1.37 (2H, q, J=7.3 Hz), 1.57 (3H, s), 1.65 (6H, s), 1.86-1.95 (2H, m), 2.12-2.23 (2H, m), 3.51-3.53 (4H, m), 4.59 (2H, s), 5.65 (1H, s), 5.84-5.90 (1H, m), 5.96 (1H, s), 6.48 (2H, d, J=4.4 Hz), 6.68 (1H, d, J=7.7 Hz), 7.15-7.18 (1H, d, J=6.0 Hz), 7.20 (1H, t, J=8.0 Hz), 7.33 (1H, d, J=8.3 Hz), 7.52 (1H, d, J=7.5 Hz), 7.72 (1H, d, J=6.0 Hz)

### Example Number 104

### 1-(((1-aminoisoquinolin-5-yl)(methyl)amino)methyl)-N-(chroman-6-yl)-4-(((1,6-dimethyl-2-oxo-1,2-dihydropyridin-4-yl)oxy)methyl)-2-azabicyclo[2.1.1]hexane-2-carboxamide

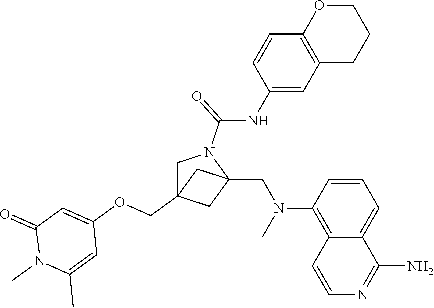 [SMILES: Cc1cc(OCC23CN(C(=O)Nc4ccc5c(c4)CCCO5)C(CN(C)c4cccc5c(N)nccc45)(C2)C3)cc(=O)n1C]

### tert-Butyl 4-[[tert-butyl(diphenyl)silyl]oxymethyl]-1-[[[1-[(2,4-dimethoxyphenyl)methylamino]-5-isoquinolyl]-methyl-amino]methyl]-2-azabicyclo[2.1.1]hexane-2-carboxylate

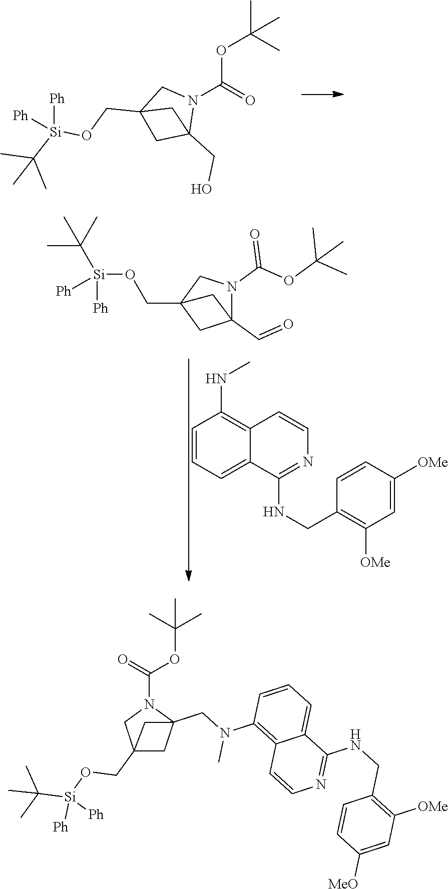

Dess-Martin periodinane (524 mg, 1.17 mmol) was added to a solution of tert-butyl 4-[[tert-butyl(diphenyl)silyl]oxymethyl]-1-(hydroxymethyl)-2-azabicyclo[2.1.1]hexane-2-carboxylate (400 mg, 0.748 mmol) in DCM (7 mL) at 0 °C and stirred for 15 min. The mixture was warmed to rt and stirred for 3 h. Sat. NaHCO₃ (aq) (5 mL) and sat. Na₂S2O₃ (aq) (5 mL) were added. The layers were separated, and the aqueous layer was extracted with DCM (3×15 mL). The combined organic layers were washed with sat. Na₂S2O₃ (aq)(15 mL), sat. NaHCO₃ (aq)(15 mL), and brine (15 mL), dried (Na₂SO₄) and concentrated to afford the aldehyde intermediate which was used without further purification in the next step.

[M+H]⁺=478.9

AcOH (60 μL, 1.0 mmol) was added to a mixture of the aldehyde intermediate and N1-[(2,4-dimethoxyphenyl)methyl]-N5-methyl-isoquinoline-1,5-diamine (274 mg, 1.17 mmol) in DCE (7 mL) at rt and stirred for 5 min. NaBH(OAc)₃ (435 mg, 1.64 mmol) was added and the mixture stirred at rt for 16 h. The mixture was cooled in an ice/water bath and sat. NaHCO₃ (aq) (5 mL) and brine (5 mL) were added. The layers were separated and the aqueous layer extracted with DCM (3×15 mL). The combined organic layers were dried (Na₂SO₄), filtered and concentrated, and the residue purified by flash chromatography (Silica, 0-48% EtOAc in hexanes) to afford the product (184 mg, 30% yield over two steps).

[M+H]⁺=787.5

¹H NMR (DMSO-d6, 500 MHz) δ 0.92 (9H, s), 1.25 (2H, dd, J=4.4, 1.8 Hz), 1.38 (9H, s), 1.73-1.80 (2H, m), 2.78 (3H, s), 3.13 (2H, s), 3.70 (3H, s), 3.73 (2H, s), 3.82 (3H, d, J=1.3 Hz), 3.84 (2H, s), 4.60 (2H, d, J=5.6 Hz), 6.36 (1H, dd, J=8.4, 2.4 Hz), 6.55 (1H, d, J=2.3 Hz), 7.06 (1H, d, J=8.3 Hz), 7.10 (1H, dd, J=6.0, 0.8 Hz), 7.34-7.41 (6H, m), 7.41-7.47 (2H, m), 7.49-7.54 (4H, m), 7.58 (1H, t, J=5.9 Hz), 7.76 (1H, d, J=6.0 Hz), 7.99 (1H, d, J=8.1 Hz)

### tert-Butyl 1-[[[1-[(2,4-dimethoxyphenyl)methylamino]-5-isoquinolyl]-methyl-amino]methyl]-4-(hydroxymethyl)-2-azabicyclo[2.1.1]hexane-2-carboxylate

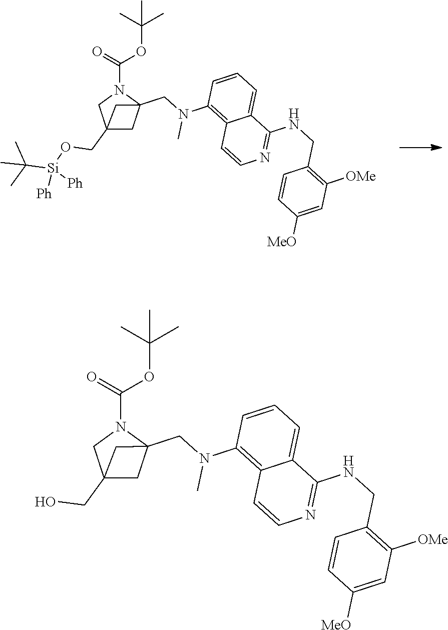 [SMILES: COc1ccc(CNc2nccc3c(N(C)CC45CC(CO[Si](c6ccccc6)(c6ccccc6)C(C)(C)C)(CN4C(=O)OC(C)(C)C)C5)cccc23)c(OC)c1 ; COc1ccc(CNc2nccc3c(N(C)CC45CC(CO)(CN4C(=O)OC(C)(C)C)C5)cccc23)c(OC)c1]

TBAF (1M in THF, 500 μL, 0.500 mmol) was added to a mixture of tert-butyl 4-[[tert-butyl(diphenyl)silyl]oxymethyl]-1-[[[1-[(2,4-dimethoxyphenyl)methylamino]-5-isoquinolyl]-methyl-amino]methyl]-2-azabicyclo[2.1.1]hexane-2-carboxylate (184 mg, 0.222 mmol) in THF (1.5 mL). The mixture was stirred at rt for 2 h then concentrated. The residue was diluted with DCM (20 mL) and washed with a mixture of sat. NaHCO₃ (aq) and brine (1:1 v/v, 10 mL). The layers were separated, and the aqueous layer was extracted with DCM (2×20 mL). The combined organic layers were dried (Na₂SO₄), filtered, and concentrated. The residue was purified by flash chromatography (Silica, 100% EtOAc) to afford the product (91 mg, 74%).

[M+H]⁺=549.8

### tert-Butyl 1-[[[1-[(2,4-dimethoxyphenyl)methylamino]-5-isoquinolyl]-methyl-amino]methyl]-4-[(1,2-dimethyl-6-oxo-4-pyridyl)oxymethyl]-2-azabicyclo[2.1.1]hexane-2-carboxylate

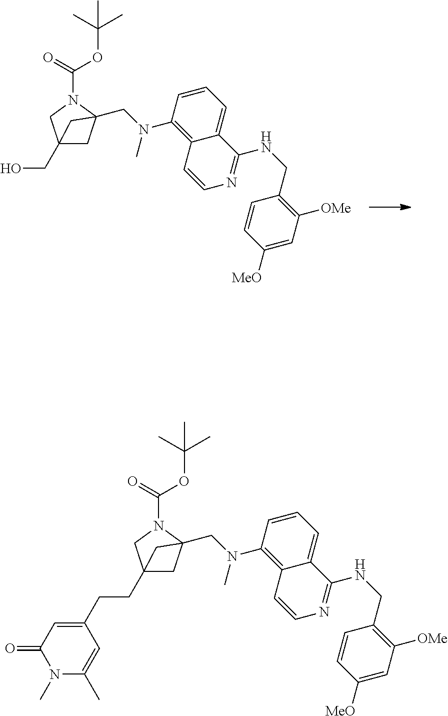 [SMILES: COc1ccc(CNc2nccc3c(N(C)CC45CC(CO)(CN4C(=O)OC(C)(C)C)C5)cccc23)c(OC)c1 ; COc1ccc(CNc2nccc3c(N(C)CC45CC(CCc6cc(C)n(C)c(=O)c6)(CN4C(=O)OC(C)(C)C)C5)cccc23)c(OC)c1]

DIAD (50 μL, 0.25 mmol) was added to a mixture of tert-butyl 1-[[[1-[(2,4-dimethoxyphenyl)methylamino]-5-isoquinolyl]-methyl-amino]methyl]-4-(hydroxymethyl)-2-azabicyclo[2.1.1]hexane-2-carboxylate (91 mg, 0.17 mmol), triphenylphospine (68 mg, 0.26 mmol) and 4-hydroxy-1,6-dimethyl-pyridin-2-one (28 mg, 0.19 mmol) in THF (2 mL) at rt. The mixture was stirred at rt for 18 h and filtered (Celite®). The solid was washed with EtOAc and the filtrate was concentrated. The residue was purified by flash chromatography (silica, 0-100% hexanes in EtOAc followed by 0-18% MeOH in EtOAc) to afford the product (75 mg, 68% yield).

[M+H]⁺=670.8

### 4-[[4-[[[1-[(2,4-Dimethoxyphenyl)methylamino]-5-isoquinolyl]-methyl-amino]methyl]-2-azabicyclo[2.1.1]hexan-1-yl]methoxy]-1,6-dimethyl-pyridin-2-one

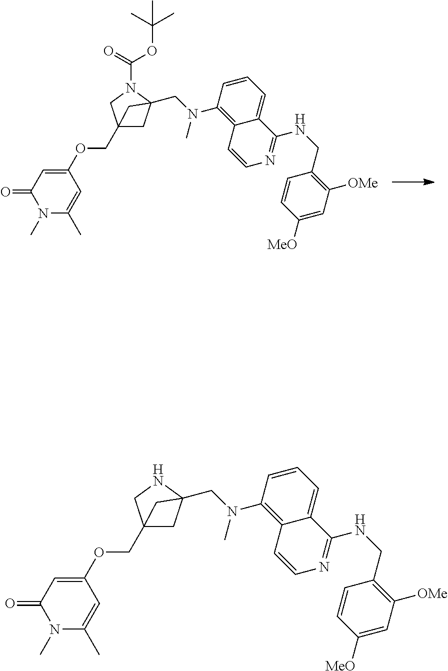 [SMILES: COc1ccc(CNc2nccc3c(N(C)CC45CC(COc6cc(C)n(C)c(=O)c6)(CN4C(=O)OC(C)(C)C)C5)cccc23)c(OC)c1 ; COc1ccc(CNc2nccc3c(N(C)CC45CC(COc6cc(C)n(C)c(=O)c6)(CN4)C5)cccc23)c(OC)c1]

HCl (1M in EtOAc, 2 mL, 2 mmol) was added to a mixture of tert-butyl 4-[[[1-[(2,4-dimethoxyphenyl)methylamino]-5-isoquinolyl]-methyl-amino]methyl]-1-[(1,2-dimethyl-6-oxo-4-pyridyl)oxymethyl]-2-azabicyclo[2.1.1]hexane-2-carboxylate (75 mg, 0.11 mmol) at rt The mixture was heated to 30 °C and stirred for 18 h and concentrated. The solid was diluted with a mixture of DCM and MeOH (9:1 v/v, 20 mL) and with NaOH (aq) (2M, 5 mL). The phases were separated, and the aqueous phase was extracted with a mixture of DCM and MeOH (9:1, 2×20 mL). The combined organic layers were dried (Na₂SO₄), filtered, and concentrated to afford the product (63 mg, 99% yield).

[M+H]⁺=570.3

### 1-(((1-aminoisoquinolin-5-yl)(methyl)amino)methyl)-N-(chroman-6-yl)-4-(((1,6-dimethyl-2-oxo-1,2-dihydropyridin-4-yl)oxy)methyl)-2-azabicyclo[2.1.1]hexane-2-carboxamide

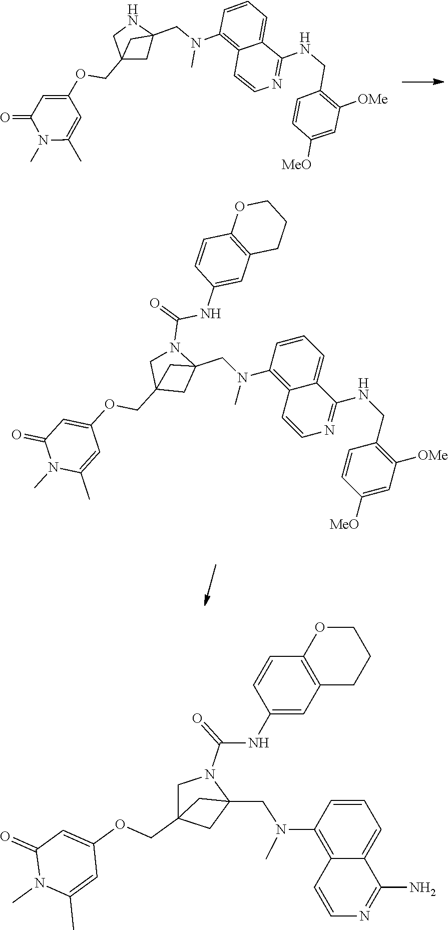 [SMILES: COc1ccc(CNc2nccc3c(N(C)CC45CC(COc6cc(C)n(C)c(=O)c6)(CN4)C5)cccc23)c(OC)c1 ; COc1ccc(CNc2nccc3c(N(C)CC45CC(COc6cc(C)n(C)c(=O)c6)(CN4C(=O)Nc4ccc6c(c4)CCCO6)C5)cccc23)c(OC)c1 ; Cc1cc(OCC23CN(C(=O)Nc4ccc5c(c4)CCCO5)C(CN(C)c4cccc5c(N)nccc45)(C2)C3)cc(=O)n1C]

Chroman-6-amine (24 mg, 0.15 mmol) in a mixture of THF (500 μL) and DCM (500 μL) was added to a mixture of triphosgene (16.4 mg, 0.0542 mmol) and pyridine (20 μL, 0.245 mmol) in THF (500 μL) at rt. The mixture was stirred at rt for 4 h and added to a mixture of 4-[[1-[[[1-[(2,4-dimethoxyphenyl)methylamino]-5-isoquinolyl]-methyl-amino]methyl]-2-azabicyclo[2.1.1]hexan-4-yl]methoxy]-1,6-dimethyl-pyridin-2-one (63 mg, 0.11 mmol) and TEA (30 μL, 0.22 mmol) in DCM (1 mL) at rt. The mixture was stirred for 1 h and concentrated to afford the intermediate N-chroman-6-yl-1-[[[1-[(2,4-dimethoxyphenyl)methylamino]-5-isoquinolyl]-methyl-amino]methyl]-4-[(1,2-dimethyl-6-oxo-4-pyridyl)oxymethyl]-2-azabicyclo[2.1.1]hexane-2-carboxamide.

[M+H]⁺=745.7

TFA (250 μL, 3.33 mmol) was added to a mixture of intermediate N-chroman-6-yl-1-[[[1-[(2,4-dimethoxyphenyl)methylamino]-5-isoquinolyl]-methyl-amino]methyl]-4-[(1,2-dimethyl-6-oxo-4-pyridyl)oxymethyl]-2-azabicyclo[2.1.1]hexane-2-carboxamide in DCM (2 mL) at rt. The mixture was stirred at rt for 18 h and diluted with NaOH (aq) (2M, 10 mL) and a mixture of DCM and MeOH (9:1 v/v, 20 mL). The phases were separated, and the aqueous phase was extracted with a mixture of DCM and MeOH (9:1 v/v, 2×20 mL). The combined organic phases were concentrated, and the residue was purified by automated prep HPLC (UV directed 34-44% MeCN in 10 mM NH₄HCO₃ (aq) over 9 min) and lyophilized to afford the product (26 mg, 40% yield over two steps).

[M+H]⁺=595.5

¹H NMR (DMSO-d6, 500 MHz) δ 1.43 (2H, dd, J=4.6, 1.7 Hz), 1.75-1.81 (2H, m), 1.83-1.89 (2H, m), 2.27 (3H, s), 2.61 (2H, t, J=6.5 Hz), 2.81 (3H, s), 3.37 (2H, s), 3.91 (2H, s), 4.02-4.06 (2H, m), 4.10 (2H, s), 5.67 (1H, d, J=2.8 Hz), 5.82 (1H, dd, J=2.8, 0.9 Hz), 6.52-6.58 (1H, m), 6.68 (2H, s), 7.01-7.08 (2H, m), 7.11-7.16 (1H, m), 7.37 (1H, t, J=7.9 Hz), 7.42 (1H, dd, J=7.6, 1.1 Hz), 7.78 (1H, d, J=6.0 Hz), 7.87 (1H, d, J=8.2 Hz), 8.56 (1H, s); 3H under the water peak.

### EXAMPLES

TABLE 6
Compound Names

| Example | |
|---|---|
| No | Name |
| 1 | N5-((4-(5-phenyloxazol-2-yl)-2-oxabicyclo[2.1.1]hexan-1-yl)methyl)isoquinoline- |
| | 1,5-diamine |
| 2 | (S)-1-((1-(((1-aminoisoquinolin-5-yl)amino)methyl)-2-oxabicyclo[2.1.1]hexan-4- |
| | yl)methyl)-5-phenylpyrrolidin-2-one |
| 3 | 4-((1-(((1-aminoisoquinolin-5-yl)amino)methyl)-2-(2-(2,3-dihydrobenzofuran-5- |
| | yl)acetyl)-2-azabicyclo[2.1.1]hexan-4-yl)methoxy)-1-methylpyridin-2(1H)-one |
| 4 | 4-((1-(((1-aminoisoquinolin-5-yl)amino)methyl)-2-(2-methyl-2- |
| | phenoxypropanoyl)-2-azabicyclo[2.1.1]hexan-4-yl)methoxy)-1-methylpyridin- |
| | 2(1H)-one |
| 5 | 4-((1-(((1-aminoisoquinolin-5-yl)amino)methyl)-2-(2-cyclohexylacetyl)-2- |
| | azabicyclo[2.1.1]hexan-4-yl)methoxy)-1-methylpyridin-2(1H)-one |
| 6 | 4-((1-(((1-aminoisoquinolin-5-yl)amino)methyl)-2-(1-phenoxycyclopropane-1- |
| | carbonyl)-2-azabicyclo[2.1.1]hexan-4-yl)methoxy)-1-methylpyridin-2(1H)-one |
| 7 | 4-((1-(((1-aminoisoquinolin-5-yl)amino)methyl)-2-(2,2-dimethyl-3- |
| | phenylpropanoyl)-2-azabicyclo[2.1.1]hexan-4-yl)methoxy)-1-methylpyridin-2(1H)- |
| | one |
| 8 | 4-((1-(((1-aminoisoquinolin-5-yl)amino)methyl)-2-(1-benzylcyclopropane-1- |
| | carbonyl)-2-azabicyclo[2.1.1]hexan-4-yl)methoxy)-1-methylpyridin-2(1H)-one |
| 9 | 4-((1-(((1-aminoisoquinolin-5-yl)amino)methyl)-2-(2-(tetrahydro-2H-pyran-4- |
| | yl)acetyl)-2-azabicyclo[2.1.1]hexan-4-yl)methoxy)-1-methylpyridin-2(1H)-one |
| 10 | (R)-4-((1-(((1-aminoisoquinolin-5-yl)amino)methyl)-2-(2-(tetrahydrofuran-3- |
| | yl)acetyl)-2-azabicyclo[2.1.1]hexan-4-yl)methoxy)-1-methylpyridin-2(1H)-one |
| 11 | (S)-4-((1-(((1-aminoisoquinolin-5-yl)amino)methyl)-2-(2-(tetrahydrofuran-3- |
| | yl)acetyl)-2-azabicyclo[2.1.1]hexan-4-yl)methoxy)-1-methylpyridin-2(1H)-one |
| 12 | (R)-4-((1-(((1-aminoisoquinolin-5-yl)amino)methyl)-2-(2-phenylpropanoyl)-2- |
| | azabicyclo[2.1.1]hexan-4-yl)methoxy)-1-methylpyridin-2(1H)-one |
| 13 | (S)-4-((1-(((1-aminoisoquinolin-5-yl)amino)methyl)-2-(2-phenylpropanoyl)-2- |
| | azabicyclo[2.1.1]hexan-4-yl)methoxy)-1-methylpyridin-2(1H)-one |
| 14 | 4-((1-(((1-aminoisoquinolin-5-yl)amino)methyl)-2-(3-(2,3-dihydrobenzofuran-5- |
| | yl)propanoyl)-2-azabicyclo[2.1.1]hexan-4-yl)methoxy)-1-methylpyridin-2(1H)-one |
| 15 | 4-((1-(((1-aminoisoquinolin-5-yl)amino)methyl)-2-(2-(chroman-6-yl)acetyl)-2- |
| | azabicyclo[2.1.1]hexan-4-yl)methoxy)-1-methylpyridin-2(1H)-one |
| 16 | 4-((2-(2-(1-acetylpiperidin-4-yl)acetyl)-1-(((1-aminoisoquinolin-5- |
| | yl)amino)methyl)-2-azabicyclo[2.1.1]hexan-4-yl)methoxy)-1-methylpyridin-2(1H)- |
| | one |
| 17 | 6-(2-(1-(((1-aminoisoquinolin-5-yl)amino)methyl)-4-(((1-methyl-2-oxo-1,2- |
| | dihydropyridin-4-yl)oxy)methyl)-2-azabicyclo[2.1.1]hexan-2-yl)-2-oxoethyl)-2H- |
| | benzo[b][1,4]oxazin-3(4H)-one |
| 18 | 4-((1-(((1-aminoisoquinolin-5-yl)amino)methyl)-2-(1-phenylcyclopropane-1- |
| | carbonyl)-2-azabicyclo[2.1.1]hexan-4-yl)methoxy)-1-methylpyridin-2(1H)-one |
| 19 | 4-((1-(((1-aminoisoquinolin-5-yl)amino)methyl)-2-(3-(1-methyl-2-oxo-1,2- |
| | dihydropyridin-4-yl)propanoyl)-2-azabicyclo[2.1.1]hexan-4-yl)methoxy)-1- |
| | methylpyridin-2(1H)-one |
| 20 | 4-((1-(((1-aminoisoquinolin-5-yl)amino)methyl)-2-(7-oxaspiro[3.5]nonane-2- |
| | carbonyl)-2-azabicyclo[2.1.1]hexan-4-yl)methoxy)-1,6-dimethylpyridin-2(1H)-one |
| 21 | 4-((1-(((1-aminoisoquinolin-5-yl)amino)methyl)-2-(2-(bicyclo[2.2.2]octan-1- |
| | yl)acetyl)-2-azabicyclo[2.1.1]hexan-4-yl)methoxy)-1,6-dimethylpyridin-2(1H)-one |
| 22 | 4-((1-(((1-aminoisoquinolin-5-yl)amino)methyl)-2-(2-(bicyclo[2.2.1]heptan-1- |
| | yl)acetyl)-2-azabicyclo[2.1.1]hexan-4-yl)methoxy)-1,6-dimethylpyridin-2(1H)-one |
| 23 | 4-((1-(((1-aminoisoquinolin-5-yl)amino)methyl)-2-(2-(4,4- |
| | difluorocyclohexyl)acetyl)-2-azabicyclo[2.1.1]hexan-4-yl)methoxy)-1,6- |
| | dimethylpyridin-2(1H)-one |
| 24 | 4-((1-(((1-aminoisoquinolin-5-yl)amino)methyl)-2-(2-((1r,4r)-4- |
| | methoxycyclohexyl)acetyl)-2-azabicyclo[2.1.1]hexan-4-yl)methoxy)-1,6- |
| | dimethylpyridin-2(1H)-one |
| 25 | 4-((1-(((1-aminoisoquinolin-5-yl)amino)methyl)-2-(2-((1s,4s)-4- |
| | methoxycyclohexyl)acetyl)-2-azabicyclo[2.1.1]hexan-4-yl)methoxy)-1,6- |
| | dimethylpyridin-2(1H)-one |
| 26 | 4-((1-(((1-aminoisoquinolin-5-yl)amino)methyl)-2-(2-(bicyclo[1.1.1]pentan-1- |
| | yl)acetyl)-2-azabicyclo[2.1.1]hexan-4-yl)methoxy)-1,6-dimethylpyridin-2(1H)-one |
| 27 | 4-((1-(((1-aminoisoquinolin-5-yl)amino)methyl)-2-(2-(2,3-dihydrobenzofuran-5- |
| | yl)propanoyl)-2-azabicyclo[2.1.1]hexan-4-yl)methoxy)-1-methylpyridin-2(1H)-one |
| 28 | 4-((1-(((1-aminoisoquinolin-5-yl)amino)methyl)-2-(2-(2,3-dihydrobenzofuran-5- |
| | yl)propanoyl)-2-azabicyclo[2.1.1]hexan-4-yl)methoxy)-1,6-dimethylpyridin-2(1H)- |
| | one |
| 29 | N-(1-(1-(((1-aminoisoquinolin-5-yl)amino)methyl)-4-(((1,6-dimethyl-2-oxo-1,2- |
| | dihydropyridin-4-yl)oxy)methyl)-2-azabicyclo[2.1.1]hexan-2-yl)-1-oxopropan-2- |
| | yl)acetamide |
| 30 | 1-(((1-aminoisoquinolin-5-yl)amino)methyl)-4-(((1-methyl-2-oxo-1,2- |
| | dihydropyridin-4-yl)oxy)methyl)-N-(1-phenylcyclopropyl)-2- |
| | azabicyclo[2.1.1]hexane-2-carboxamide |
| 31 | 4-((1-(((1-aminoisoquinolin-5-yl)amino)methyl)-2-(morpholine-4-carbonyl)-2- |
| | azabicyclo[2.1.1]hexan-4-yl)methoxy)-1-methylpyridin-2(1H)-one |
| 32 | (S)-1-(((1-aminoisoquinolin-5-yl)amino)methyl)-4-(((1-methyl-2-oxo-1,2- |
| | dihydropyridin-4-yl)oxy)methyl)-N-(1-phenylethyl)-2-azabicyclo[2.1.1]hexane-2- |
| | carboxamide |
| 33 | (R)-1-(((1-aminoisoquinolin-5-yl)amino)methyl)-4-(((1-methyl-2-oxo-1,2- |
| | dihydropyridin-4-yl)oxy)methyl)-N-(1-phenylethyl)-2-azabicyclo[2.1.1]hexane-2- |
| | carboxamide |
| 34 | 4-((1-(((1-aminoisoquinolin-5-yl)amino)methyl)-2-(indoline-1-carbonyl)-2- |
| | azabicyclo[2.1.1]hexan-4-yl)methoxy)-1-methylpyridin-2(1H)-one |
| 35 | 4-((1-(((1-aminoisoquinolin-5-yl)amino)methyl)-2-(3,4-dihydro-2H- |
| | benzo[b][1,4]oxazine-4-carbonyl)-2-azabicyclo[2.1.1]hexan-4-yl)methoxy)-1- |
| | methylpyridin-2(1H)-one |
| 36 | 4-((1-(((1-aminoisoquinolin-5-yl)amino)methyl)-2-(1,2,3,4-tetrahydroquinoline-1- |
| | carbonyl)-2-azabicyclo[2.1.1]hexan-4-yl)methoxy)-1-methylpyridin-2(1H)-one |
| 37 | 4-((1-(((1-aminoisoquinolin-5-yl)amino)methyl)-2-(1,2,3,4-tetrahydroisoquinoline- |
| | 2-carbonyl)-2-azabicyclo[2.1.1]hexan-4-yl)methoxy)-1-methylpyridin-2(1H)-one |
| 38 | 1-(((1-aminoisoquinolin-5-yl)amino)methyl)-N-(2,3-dihydrobenzofuran-5-yl)-4- |
| | (((1-methyl-2-oxo-1,2-dihydropyridin-4-yl)oxy)methyl)-2-azabicyclo[2.1.1]hexane- |
| | 2-carboxamide |
| 39 | 4-((1-(((1-aminoisoquinolin-5-yl)amino)methyl)-2-(6-methoxy-1,2,3,4- |
| | tetrahydroquinoline-1-carbonyl)-2-azabicyclo[2.1.1]hexan-4-yl)methoxy)-1- |
| | methylpyridin-2(1H)-one |
| 40 | (R)-1-(((1-aminoisoquinolin-5-yl)amino)methyl)-4-(((1,6-dimethyl-2-oxo-1,2- |
| | dihydropyridin-4-yl)oxy)methyl)-N-methyl-N-(tetrahydrofuran-3-yl)-2- |
| | azabicyclo [2.1.1]hexane-2-carboxamide |
| 41 | (S)-1-(((1-aminoisoquinolin-5-yl)amino)methyl)-4-(((1,6-dimethyl-2-oxo-1,2- |
| | dihydropyridin-4-yl)oxy)methyl)-N-methyl-N-(tetrahydrofuran-3-yl)-2- |
| | azabicyclo[2.1.1]hexane-2-carboxamide |
| 42 | (S)-1-(((1-aminoisoquinolin-5-yl)amino)methyl)-N-methyl-4-(((1-methyl-2-oxo-1,2- |
| | dihydropyridin-4-yl)oxy)methyl)-N-(1-phenylethyl)-2-azabicyclo[2.1.1]hexane-2- |
| | carboxamide |
| 43 | (R)-1-(((1-aminoisoquinolin-5-yl)amino)methyl)-N-methyl-4-(((1-methyl-2-oxo- |
| | 1,2-dihydropyridin-4-yl)oxy)methyl)-N-(1-phenylethyl)-2-azabicyclo[2.1.1]hexane- |
| | 2-carboxamide |
| 44 | (S)-4-((1-(((1-aminoisoquinolin-5-yl)amino)methyl)-2-(3-phenylpyrrolidine-1- |
| | carbonyl)-2-azabicyclo[2.1.1]hexan-4-yl)methoxy)-1-methylpyridin-2(1H)-one |
| 45 | (S)-4-((1-(((1-aminoisoquinolin-5-yl)amino)methyl)-2-(3-phenylpyrrolidine-1- |
| | carbonyl)-2-azabicyclo[2.1.1]hexan-4-yl)methoxy)-1,6-dimethylpyridin-2(1H)-one |
| 46 | 4-((1-(((1-aminoisoquinolin-5-yl)amino) methyl)-2-(7-oxa-2-azaspiro[3.5]nonane- |
| | 2-carbonyl)-2-azabicyclo[2.1.1]hexan-4-yl)methoxy)-1,6-dimethylpyridin-2(1H)- |
| | one |
| 47 | (S)-1-(((1-aminoisoquinolin-5-yl)amino)methyl)-4-(((1,6-dimethyl-2-oxo-1,2- |
| | dihydropyridin-4-yl)oxy)methyl)-N-methyl-N-((tetrahydrofuran-3-yl)methyl)-2- |
| | azabicyclo[2.1.1]hexane-2-carboxamide |
| 48 | (R)-1-(((1-aminoisoquinolin-5-yl)amino)methyl)-4-(((1,6-dimethyl-2-oxo-1,2- |
| | dihydropyridin-4-yl)oxy)methyl)-N-methyl-N-((tetrahydrofuran-3-yl)methyl)-2- |
| | azabicyclo[2.1.1]hexane-2-carboxamide |
| 49 | (R)-4-((1-(((1-aminoisoquinolin-5-yl)amino)methyl)-2-(3-phenylpyrrolidine-1- |
| | carbonyl)-2-azabicyclo[2.1.1]hexan-4-yl)methoxy)-1,6-dimethylpyridin-2(1H)-one |
| 50 | 4-((1-(((1-aminoisoquinolin-5-yl)amino)methyl)-2-(isoindoline-2-carbonyl)-2- |
| | azabicyclo[2.1.1]hexan-4-yl)methoxy)-1-methylpyridin-2(1H)-one |
| 51 | 4-((1-(((1-aminoisoquinolin-5-yl)amino)methyl)-2-(2-azaspiro[3.3]heptane-2- |
| | carbonyl)-2-azabicyclo[2.1.1]hexan-4-yl)methoxy)-1-methylpyridin-2(1H)-one |
| 52 | 1-(((1-aminoisoquinolin-5-yl)amino)methyl)-N-(chroman-6-yl)-4-(((1-methyl-2- |
| | oxo-1,2-dihydropyridin-4-yl)oxy)methyl)-2-azabicyclo[2.1.1]hexane-2- |
| | carboxamide |
| 53 | 1-(((1-aminoisoquinolin-5-yl)amino)methyl)-N-(cyclopropylmethyl)-N-methyl-4- |
| | (((1-methyl-2-oxo-1,2-dihydropyridin-4-yl)oxy)methyl)-2-azabicyclo[2.1.1]hexane- |
| | 2-carboxamide |
| 54 | 4-((1-(((1-aminoisoquinolin-5-yl)amino)methyl)-2-(piperidine-1-carbonyl)-2- |
| | azabicyclo[2.1.1]hexan-4-yl)methoxy)-1-methylpyridin-2(1H)-one |
| 55 | 4-(((1,6-dimethyl-2-oxo-1,2-dihydropyridin-4-yl)oxy)methyl)-N-(1- |
| | phenylcyclopropyl)-1-((pyridin-3-ylamino)methyl)-2-azabicyclo[2.1.1]hexane-2- |
| | carboxamide |
| 56 | 4-(((1,6-dimethyl-2-oxo-1,2-dihydropyridin-4-yl)oxy)methyl)-1-(((2-methylpyridin- |
| | 3-yl)amino)methyl)-N-(1-phenylcyclopropyl)-2-azabicyclo[2.1.1]hexane-2- |
| | carboxamide |
| 57 | 1-(((1-aminoisoquinolin-5-yl)amino)methyl)-4-(((1,6-dimethyl-2-oxo-1,2- |
| | dihydropyridin-4-yl)oxy)methyl)-N-(1-phenylcyclopropyl)-2- |
| | azabicyclo[2.1.1]hexane-2-carboxamide |
| 58 | 1-(((6-aminopyridin-3-yl)amino)methyl)-4-(((1,6-dimethyl-2-oxo-1,2- |
| | dihydropyridin-4-yl)oxy)methyl)-N-(1-phenylcyclopropyl)-2- |
| | azabicyclo[2.1.1]hexane-2-carboxamide |
| 59 | 1-(((1-aminoisoquinolin-5-yl)amino)methyl)-4-(((5-oxo-1,2,3,5- |
| | tetrahydroindolizin-7-yl)oxy)methyl)-N-(1-phenylcyclopropyl)-2- |
| | azabicyclo[2.1.1]hexane-2-carboxamide |
| 60 | rac-1-(((1-aminoisoquinolin-5-yl)amino)methyl)-4-(((1-methyl-2-oxo-1,2- |
| | dihydropyridin-4-yl)oxy)methyl)-N-((1R,2S)-2-phenylcyclopropyl)-2- |
| | azabicyclo[2.1.1]hexane-2-carboxamide |
| 61 | N-(adamantan-2-yl)-1-(((1-aminoisoquinolin-5-yl)amino)methyl)-4-(((1-methyl-2- |
| | oxo-1,2-dihydropyridin-4-yl)oxy)methyl)-2-azabicyclo[2.1.1]hexane-2- |
| | carboxamide |
| 62 | 1-(((1-aminoisoquinolin-5-yl)amino)methyl)-N-(6-fluoro-4H-benzo[d][1,3]dioxin- |
| | 8-yl)-4-(((1-methyl-2-oxo-1,2-dihydropyridin-4-yl)oxy)methyl)-2- |
| | azabicyclo [2.1.1]hexane-2-carboxamide |
| 63 | 1-(((1-aminoisoquinolin-5-yl)amino)methyl)-4-(((1-methyl-2-oxo-1,2- |
| | dihydropyridin-4-yl)oxy)methyl)-2-azabicyclo[2.1.1]hexane-2-carboxamide |
| 64 | 1-(((1-aminoisoquinolin-5-yl)amino)methyl)-4-(((1-methyl-2-oxo-1,2- |
| | dihydropyridin-4-yl)oxy)methyl)-N-(4-phenyltetrahydro-2H-pyran-4-yl)-2- |
| | azabicyclo[2.1.1]hexane-2-carboxamide |
| 65 | 1-(((1-aminoisoquinolin-5-yl)amino)methyl)-N-(2,3-dihydro-1H-inden-5-yl)-4- |
| | (((1,6-dimethyl-2-oxo-1,2-dihydropyridin-4-yl)oxy)methyl)-2- |
| | azabicyclo[2.1.1]hexane-2-carboxamide |
| 66 | 1-(((1-aminoisoquinolin-5-yl)amino)methyl)-4-(((1-methyl-2-oxo-1,2- |
| | dihydropyridin-4-yl)oxy)methyl)-N-(2-phenylpropan-2-yl)-2- |
| | azabicyclo[2.1.1]hexane-2-carboxamide |
| 67 | 4-(((1,6-dimethyl-2-oxo-1,2-dihydropyridin-4-yl)oxy)methyl)-1-(((2-fluoropyridin- |
| | 3-yl)amino)methyl)-N-(1-phenylcyclopropyl)-2-azabicyclo[2.1.1]hexane-2- |
| | carboxamide |
| 68 | 1-(((2-cyanophenyl)amino)methyl)-4-(((1,6-dimethyl-2-oxo-1,2-dihydropyridin-4- |
| | yl)oxy)methyl)-N-(1-phenylcyclopropyl)-2-azabicyclo[2.1.1]hexane-2-carboxamide |
| 69 | 1-(((1-aminoisoquinolin-5-yl)amino)methyl)-4-(((1-methyl-2-oxo-1,2- |
| | dihydropyridin-4-yl)oxy)methyl)-N-(3-phenyloxetan-3-yl)-2- |
| | azabicyclo [2.1.1]hexane-2-carboxamide |
| 70 | 4-((1-(((1-aminoisoquinolin-5-yl)amino)methyl)-2-(4-phenylpiperidine-1- |
| | carbonyl)-2-azabicyclo[2.1.1]hexan-4-yl)methoxy)-1,6-dimethylpyridin-2(1H)-one |
| 71 | 4-(((1,6-dimethyl-2-oxo-1,2-dihydropyridin-4-yl)oxy)methyl)-1-(((2- |
| | hydroxypyridin-3-yl)amino)methyl)-N-(1-phenylcyclopropyl)-2- |
| | azabicyclo[2.1.1]hexane-2-carboxamide |
| 72 | 4-((1-(((1-aminoisoquinolin-5-yl)amino)methyl)-2-(3-(tetrahydro-2H-pyran-4- |
| | yl)azetidine-1-carbonyl)-2-azabicyclo[2.1.1]hexan-4-yl)methoxy)-1,6- |
| | dimethylpyridin-2(1H)-one |
| 73 | 1-(((1-aminoisoquinolin-5-yl)amino)methyl)-N-(2-(2-methoxyethoxy)ethyl)-4-(((1- |
| | methyl-2-oxo-1,2-dihydropyridin-4-yl)oxy)methyl)-2-azabicyclo[2.1.1]hexane-2- |
| | carboxamide |
| 74 | 1-(((1-aminoisoquinolin-5-yl)amino)methyl)-N-(4-(cyclohexylsulfonyl)cyclohexyl)- |
| | 4-(((1-methyl-2-oxo-1,2-dihydropyridin-4-yl)oxy)methyl)-2- |
| | azabicyclo[2.1.1]hexane-2-carboxamide |
| 75 | 1-(((1-aminoisoquinolin-5-yl)amino)methyl)-4-(((1-methyl-2-oxo-1,2- |
| | dihydropyridin-4-yl)oxy)methyl)-N-(spiro[3.3]heptan-2-yl)-2- |
| | azabicyclo[2.1.1]hexane-2-carboxamide |
| 76 | 1-(((1-aminoisoquinolin-5-yl)amino)methyl)-4-(((1-methyl-2-oxo-1,2- |
| | dihydropyridin-4-yl)oxy)methyl)-N-((3-methyloxetan-3-yl)methyl)-2- |
| | azabicyclo[2.1.1]hexane-2-carboxamide |
| 77 | 1-(((1-aminoisoquinolin-5-yl)amino)methyl)-4-(((1-methyl-2-oxo-1,2- |
| | dihydropyridin-4-yl)oxy)methyl)-N-((tetrahydro-2H-pyran-4-yl)methyl)-2- |
| | azabicyclo[2.1.1]hexane-2-carboxamide |
| 78 | 1-(((1-aminoisoquinolin-5-yl)amino)methyl)-4-(((1-methyl-2-oxo-1,2- |
| | dihydropyridin-4-yl)oxy)methyl)-N-(4-methyltetrahydro-2H-pyran-4-yl)-2- |
| | azabicyclo[2.1.1]hexane-2-carboxamide |
| 79 | 1-(((1-aminoisoquinolin-5-yl)amino)methyl)-4-(((1-methyl-2-oxo-1,2- |
| | dihydropyridin-4-yl)oxy)methyl)-N-(3-methyloxetan-3-yl)-2- |
| | azabicyclo[2.1.1]hexane-2-carboxamide |
| 80 | 1-(((1-aminoisoquinolin-5-yl)amino)methyl)-4-(((1-methyl-2-oxo-1,2- |
| | dihydropyridin-4-yl)oxy)methyl)-N-(2,2,3,3-tetramethylcyclopropyl)-2- |
| | azabicyclo [2.1.1]hexane-2-carboxamide |
| 81 | 1-(((1-aminoisoquinolin-5-yl)amino)methyl)-N-(2-cyclobutylethyl)-4-(((1-methyl-2- |
| | oxo-1,2-dihydropyridin-4-yl)oxy)methyl)-2-azabicyclo[2.1.1]hexane-2- |
| | carboxamide |
| 82 | 1-(((1-aminoisoquinolin-5-yl)amino)methyl)-N-(3-(benzyloxy)propyl)-4-(((1- |
| | methyl-2-oxo-1,2-dihydropyridin-4-yl)oxy)methyl)-2-azabicyclo[2.1.1]hexane-2- |
| | carboxamide |
| 83 | 4-((1-(((1-aminoisoquinolin-5-yl)amino)methyl)-2-(8-oxa-2-azaspiro[4.5]decane-2- |
| | carbonyl)-2-azabicyclo[2.1.1]hexan-4-yl)methoxy)-1,6-dimethylpyridin-2(1H)-one |
| 84 | 4-((1-(((1-aminoisoquinolin-5-yl)amino)methyl)-2-(5-azaspiro[2.5]octane-5- |
| | carbonyl)-2-azabicyclo[2.1.1]hexan-4-yl)methoxy)-1,6-dimethylpyridin-2(1H)-one |
| 85 | 4-((1-(((1-aminoisoquinolin-5-yl)amino)methyl)-2-(2-azaspiro[4.4]nonane-2- |
| | carbonyl)-2-azabicyclo[2.1.1]hexan-4-yl)methoxy)-1,6-dimethylpyridin-2(1H)-one |
| 86 | 4-((1-(((1-aminoisoquinolin-5-yl)amino)methyl)-2-(3-(3- |
| | methoxyphenyl)pyrrolidine-1-carbonyl)-2-azabicyclo[2.1.1]hexan-4-yl)methoxy)- |
| | 1,6-dimethylpyridin-2(1H)-one |
| 87 | 4-((1-(((1-aminoisoquinolin-5-yl)amino)methyl)-2-(1,1-difluoro-5- |
| | azaspiro[2.5]octane-5-carbonyl)-2-azabicyclo[2.1.1]hexan-4-yl)methoxy)-1,6- |
| | dimethylpyridin-2(1H)-one |
| 88 | (S)-1-(((1-aminoisoquinolin-5-yl)amino)methyl)-4-(((1-methyl-2-oxo-1,2- |
| | dihydropyridin-4-yl)oxy)methyl)-N-(2,2,2-trifluoro-1-phenylethyl)-2- |
| | azabicyclo [2.1.1]hexane-2-carboxamide |
| 89 | 4-((1-(((1-aminoisoquinolin-5-yl)amino)methyl)-2-(1,1-difluoro-5- |
| | azaspiro[2.4]heptane-5-carbonyl)-2-azabicyclo[2.1.1]hexan-4-yl)methoxy)-1,6- |
| | dimethylpyridin-2(1H)-one |
| 90 | 1-(((1-aminoisoquinolin-5-yl)amino)methyl)-N-(2,3-dihydrobenzofuran-3-yl)-4- |
| | (((1,6-dimethyl-2-oxo-1,2-dihydropyridin-4-yl)oxy)methyl)-N-methyl-2- |
| | azabicyclo[2.1.1]hexane-2-carboxamide |
| 91 | 1-(((1-aminoisoquinolin-5-yl)amino)methyl)-4-(((1-methyl-2-oxo-1,2- |
| | dihydropyridin-4-yl)oxy)methyl)-N-phenyl-2-azabicyclo[2.1.1]hexane-2- |
| | carbothioamide |
| 92 | 1-(((1-aminoisoquinolin-5-yl)amino)methyl)-N-(2,2-difluorobenzo[d][1,3]dioxol-5- |
| | yl)-4-(((1,6-dimethyl-2-oxo-1,2-dihydropyridin-4-yl)oxy)methyl)-2- |
| | azabicyclo[2.1.1]hexane-2-carboxamide |
| 93 | 4-((1-(((1-aminoisoquinolin-5-yl)amino)methyl)-2-(5-cyclopropylpyrimidin-2-yl)-2- |
| | azabicyclo[2.1.1]hexan-4-yl)methoxy)-1-methylpyridin-2(1H)-one |
| 94 | 4-((1-(((1-aminoisoquinolin-5-yl)amino)methyl)-2-(6-cyclopropylpyridin-3-yl)-2- |
| | azabicyclo[2.1.1]hexan-4-yl)methoxy)-1,6-dimethylpyridin-2(1H)-one |
| 95 | 4-((1-(((1-aminoisoquinolin-5-yl)amino)methyl)-2-(6-cyclopropylpyridazin-3-yl)-2- |
| | azabicyclo[2.1.1]hexan-4-yl)methoxy)-1,6-dimethylpyridin-2(1H)-one |
| 96 | 4-((1-(((1-aminoisoquinolin-5-yl)amino)methyl)-2-(4-methyl-5-phenyl-4,5- |
| | dihydrooxazol-2-yl)-2-azabicyclo[2.1.1]hexan-4-yl)methoxy)-1,6-dimethylpyridin- |
| | 2(1H)-one |
| 97 | 4-((1-(((1-aminoisoquinolin-5-yl)amino)methyl)-2-(4,5-dihydrooxazol-2-yl)-2- |
| | azabicyclo[2.1.1]hexan-4-yl)methoxy)-1,6-dimethylpyridin-2(1H)-one |
| 98 | 4-((1-(((1-aminoisoquinolin-5-yl)amino)methyl)-2-(1-cyclopropyl-1H-pyrazol-4-yl)- |
| | 2-azabicyclo[2.1.1]hexan-4-yl)methoxy)-1,6-dimethylpyridin-2(1H)-one |
| 99 | 4-((1-(((1-aminoisoquinolin-5-yl)amino)methyl)-2-(3-cyclopropyl-1,2,4-oxadiazol- |
| | 5-yl)-2-azabicyclo[2.1.1]hexan-4-yl)methoxy)-1,6-dimethylpyridin-2(1H)-one |
| 101 | 4-((1-(((1-aminoisoquinolin-5-yl)amino)methyl)-2-(6-cyclopropylpyridazin-4-yl)-2- |
| | azabicyclo[2.1.1]hexan-4-yl)methoxy)-1,6-dimethylpyridin-2(1H)-one |
| 102 | 4-((4-(((1-aminoisoquinolin-5-yl)amino)methyl)-2-(3-cyclohexylpropanoyl)-2- |
| | azabicyclo[2.1.1]hexan-1-yl)methoxy)-1-methylpyridin-2(1H)-one |
| 103 | 4-((4-(((1-aminoisoquinolin-5-yl)amino)methyl)-2-(2-cyclohexylacetyl)-2- |
| | azabicyclo[2.1.1]hexan-1-yl)methoxy)-1-methylpyridin-2(1H)-one |
| 104 | 1-(((1-aminoisoquinolin-5-yl)(methyl)amino)methyl)-N-(chroman-6-yl)-4-(((1,6- |
| | dimethyl-2-oxo-1,2-dihydropyridin-4-yl)oxy)methyl)-2-azabicyclo[2.1.1]hexane-2- |
| | carboxamide |
| 105 | 4-((1-(((1-aminoisoquinolin-5-yl)amino)methyl)-2-(5-cyclopropyl-1,3,4-oxadiazol- |
| | 2-yl)-2-azabicyclo[2.1.1]hexan-4-yl)methoxy)-1,6-dimethylpyridin-2(1H)-one |

TABLE 7
¹H NMR data of examples (solvent d6 DMSO unless otherwise indicated)

| Example | |
|---|---|
| No | NMR write-up |
| 1 | 1.94 (2H, d, J = 4.2 Hz), 2.32 (2H, d, J = 4.4 Hz), 3.63 (2H, d, J = 5.4 Hz), 4.06 (2H, s), 6.00 |
| | (1H, t, J = 5.8 Hz), 6.51 (2H, s), 6.74 (1H, d, J = 7.7 Hz), 7.14 (1H, d, J = 6.1 Hz), 7.23 (1H, t, |
| | J = 7.9 Hz), 7.32-7.40 (2H, m), 7.41-7.50 (2H, m), 7.63 (1H, s), 7.66-7.77 (3H, m) |
| 2 | 1.32-1.42 (1H, m), 1.49-1.58 (3H, m), 1.68-1.80 (1H, m), 2.28-2.48 (3H, m), 2.74 (1H, d, |
| | J = 14.5 Hz), 3.44 (2H, d, J = 5.9 Hz), 3.48 (2H, s), 3.81 (1H, d, J = 14.5 Hz), 4.61-4.69 (1H, |
| | m), 5.77 (1H, t, J = 6.0 Hz), 6.49 (2H, s), 6.63 (1H, d, J = 7.7 Hz), 7.07 (1H, d, J = 6.1 Hz), |
| | 7.15-7.24 (3H, m), 7.25-7.41 (4H, m), 7.71 (1H, d, J = 6.1 Hz) |
| 3 | 1.49 (d, J = 4.4 Hz, 2H), 1.89 (d, J = 4.7 Hz, 2H), 3.13 (t, J = 8.7 Hz, 2H), 3.31 (s, 3H), 3.55 (d, |
| | J = 2.7 Hz, 4H), 3.98 (d, J = 6.4 Hz, 2H), 4.13 (s, 2H), 4.49 (t, J = 8.7 Hz, 2H), 5.77 (d, J = 2.7 |
| | Hz, 1H), 5.88 (dd, J = 7.5, 2.7 Hz, 1H), 6.15 (t, J = 6.6 Hz, 1H), 6.51 (s, 2H), 6.68 (d, J = 8.1 |
| | Hz, 1H), 6.75-6.83 (m, 2H), 6.99 (d, J = 8.6 Hz, 1H), 7.15 (s, 1H), 7.20 (t, J = 8.0 Hz, 1H), |
| | 7.32 (d, J = 8.3 Hz, 1H), 7.53 (d, J = 7.6 Hz, 1H), 7.70 (d, J = 6.1 Hz, 1H) |
| 4 | 1.21-1.28 (2H, m), 1.58 (6H, s), 1.78-1.85 (2H, m), 3.29 (3H, s), 3.58 (2H, s), 4.02 (2H, s), |
| | 4.08 (2H, d, J = 6.6 Hz), 5.69 (1H, d, J = 2.7 Hz), 5.77 (1H, dd, J = 7.5, 2.8 Hz), 6.04 (1H, t, J = |
| | 6.8 Hz), 6.55 (2H, s), 6.79-6.93 (5H, m), 7.05-7.12 (2H, m), 7.21 (1H, t, J = 8.0 Hz), 7.35 |
| | (1H, d, J = 8.3 Hz), 7.49 (1H, d, J = 7.6 Hz), 7.77 (1H, d, J = 6.1 Hz) |
| 5 | 0.96 (2H, q, J = 11.9 Hz), 1.12 (1H, q, J = 10.9 Hz), 1.19-1.27 (2H, m), 1.43-1.52 (2H, m), |
| | 1.64 (3H, t, J = 14.7 Hz), 1.73 (2H, d, J = 12.7 Hz), 1.76-1.85 (1H, m), 1.86-1.92 (2H, m), |
| | 2.14 (2H, d, J = 6.9 Hz), 3.31 (3H, s), 3.48 (2H, s), 3.97 (2H, d, J = 6.6 Hz), 4.13 (2H, s), 5.77 |
| | (1H, d, J = 2.7 Hz), 5.89 (1H, dd, J = 7.5, 2.7 Hz), 6.21 (1H, t, J = 6.7 Hz), 6.51 (2H, s), 6.79 |
| | (1H, d, J = 6.2 Hz), 6.81 (1H, d, J = 7.9 Hz), 7.20 (1H, t, J = 8.0 Hz), 7.32 (1H, d, J = 8.3 Hz), |
| | 7.53 (1H, d, J = 7.6 Hz), 7.70 (1H, d, J = 6.1 Hz) |
| 6 | (CDCl3) 1.19-1.24 (2H, m), 1.55-1.58 (2H, m), 1.60 (2H, dd, J = 4.7, 2.0 Hz), 1.81 (2H, d, J = |
| | 4.9 Hz), 3.48 (3H, s), 3.77 (2H, s), 3.98 (2H, s), 4.05 (2H, s), 5.85-5.89 (2H, m), 6.86-6.88 |
| | (1H, m), 6.92 (1H, d, J = 8.0 Hz), 6.97-7.03 (3H, m), 7.10-7.18 (2H, m), 7.20-7.25 (2H, m), |
| | 7.42 (1H, t, J = 8.1 Hz), 7.48 (1H, d, J = 7.1 Hz), 8.45 (2H, s) |
| 7 | 1.24 (6H, s), 1.39 (2H, dd, J = 4.6, 1.9 Hz), 1.73 (2H, d, J = 4.6 Hz), 2.86 (2H, s), 3.29 (2H, s), |
| | 3.33 (3H, s), 3.95 (2H, d, J = 6.4 Hz), 4.01 (2H, s), 5.70 (1H, d, J = 2.8 Hz), 5.84 (1H, dd, J = |
| | 7.5, 2.8 Hz), 6.07 (1H, t, J = 6.5 Hz), 6.52 (2H, s), 6.69 (1H, d, J = 7.7 Hz), 6.91 (1H, d, J = 6.1 |
| | Hz), 7.17-7.28 (6H, m), 7.32 (1H, d, J = 8.3 Hz), 7.52 (1H, d, J = 7.6 Hz), 7.71 (1H, d, J = 6.0 |
| | Hz) |
| 8 | 0.73-0.79 (2H, m), 0.93-0.98 (2H, m), 1.44 (2H, dd, J = 4.5, 1.8 Hz), 1.79 (2H, d, J = 5.5 |
| | Hz), 2.89 (2H, s), 3.31 (3H, s), 3.55 (2H, s), 3.87 (2H, d, J = 6.4 Hz), 4.09 (2H, s), 5.76 (1H, d, |
| | J = 2.7 Hz), 5.89 (1H, dd, J = 7.5, 2.7 Hz), 5.96 (1H, t, J = 6.6 Hz), 6.50 (2H, s), 6.68 (1H, d, J = |
| | 7.7 Hz), 6.77 (1H, dd, J = 6.3, 0.8 Hz), 7.15-7.30 (6H, m), 7.32 (1H, d, J = 8.3 Hz), 7.54 (1H, |
| | d, J = 7.6 Hz), 7.70 (1H, d, J = 6.1 Hz) |
| 9 | 1.19-1.31 (2H, m), 1.46-1.53 (2H, m), 1.62 (2H, d, J = 12.9 Hz), 1.86-1.92 (2H, m), 1.98- |
| | 2.08 (1H, m), 2.21 (2H, d, J = 6.9 Hz), 3.28-3.29 (1H, m), 3.33-3.35 (1H, m), 3.49 (2H, s), |
| | 3.81 (2H, dd, J = 11.4, 4.2 Hz), 3.98 (2H, d, J = 6.6 Hz), 4.13 (2H, s), 5.77 (1H, d, J = 2.8 Hz), |
| | 5.89 (1H, dd, J = 7.5, 2.8 Hz), 6.20 (1H, t, J = 6.6 Hz), 6.51 (2H, s), 6.79 (1H, d, J = 6.5 Hz), |
| | 6.81 (1H, d, J = 7.9 Hz), 7.20 (1H, t, J = 8.0 Hz), 7.32 (1H, d, J = 8.3 Hz), 7.54 (1H, d, J = 7.5 |
| | Hz), 7.70 (1H, d, J = 6.0 Hz). 3H masked by water peak |
| 10 | 1.47-1.57 (3H, m), 1.85-1.92 (2H, m), 1.97-2.09 (1H, m), 2.33-2.47 (2H, m), 2.57 (1H, p, |
| | J = 7.3 Hz), 3.31 (3H, s), 3.31-3.33 (1H, m), 3.49 (2H, s), 3.60-3.67 (1H, m), 3.68-3.76 |
| | (1H, m), 3.83-3.89 (1H, m), 3.97 (2H, d, J = 6.6 Hz), 4.13 (2H, s), 5.77 (1H, d, J = 2.7 Hz), |
| | 5.88 (1H, dd, J = 7.5, 2.8 Hz), 6.19 (1H, t, J = 6.7 Hz), 6.51 (2H, s), 6.77-6.84 (2H, m), 7.21 |
| | (1H, t, J = 8.0 Hz), 7.33 (1H, d, J = 8.3 Hz), 7.53 (1H, d, J = 7.5 Hz), 7.71 (1H, d, J = 6.1 Hz) |
| 11 | 1.47-1.58 (3H, m), 1.87-1.92 (2H, m), 2.00-2.09 (1H, m), 2.33-2.47 (2H, m), 2.53-2.60 |
| | (1H, m), 3.28-3.30 (1H, m), 3.31 (3H, s), 3.49 (2H, s), 3.64 (1H, q, J = 7.6 Hz), 3.73 (1H, td, |
| | J = 8.1, 5.2 Hz), 3.86 (1H, dd, J = 8.3, 7.0 Hz), 3.97 (2H, d, J = 6.6 Hz), 4.13 (2H, s), 5.77 (1H, d, |
| | J = 2.8 Hz), 5.88 (1H, dd, J = 7.5, 2.8 Hz), 6.19 (1H, t, J = 6.7 Hz), 6.51 (2H, s), 6.78-6.84 |
| | (2H, m), 7.21 (1H, t, J = 8.0 Hz), 7.33 (1H, d, J = 8.3 Hz), 7.54 (1H, d, J = 7.5 Hz), 7.71 (1H, d, |
| | J = 6.1 Hz) |
| 12 | 1.12-1.19 (1H, m), 1.35 (3H, d, J = 6.8 Hz), 1.57 (1H, dd, J = 10.0, 7.0 Hz), 1.81 (1H, d, J = |
| | 7.0 Hz), 1.86-1.92 (1H, m), 3.22 (1H, d, J = 7.7 Hz), 3.30 (3H, s), 3.64-3.69 (1H, m), 3.89 |
| | (1H, q, J = 6.9 Hz), 4.01 (2H, d, J = 6.6 Hz), 4.07 (2H, s), 5.73 (1H, d, J = 2.8 Hz), 5.82 (1H, dd, |
| | J = 7.5, 2.8 Hz), 6.13 (1H, t, J = 6.7 Hz), 6.51 (2H, s), 6.76-6.85 (2H, m), 7.16-7.26 (2H, m), |
| | 7.27-7.35 (3H, m), 7.35-7.40 (2H, m), 7.51 (1H, d, J = 7.5 Hz), 7.72 (1H, d, J = 6.1 Hz) |
| 13 | 1.10-1.20 (m, 1H), 1.35 (d, J = 6.8 Hz, 3H), 1.51-1.60 (m, 1H), 1.81 (d, J = 7.0 Hz, 1H), 1.89 |
| | (d, J = 6.9 Hz, 1H), 3.22 (d, J = 7.7 Hz, 1H), 3.30 (s, 3H), 3.67 (d, J = 7.7 Hz, 1H), 3.89 (q, J = |
| | 6.8 Hz, 1H), 4.01 (d, J = 6.4 Hz, 2H), 4.07 (s, 2H), 5.73 (d, J = 2.8 Hz, 1H), 5.82 (dd, J = 7.5, |
| | 2.7 Hz, 1H), 6.13 (t, J = 6.7 Hz, 1H), 6.51 (s, 2H), 6.75-6.84 (m, 2H), 7.16-7.26 (m, 2H), |
| | 7.27-7.41 (m, 5H), 7.51 (d, J = 7.6 Hz, 1H), 7.72 (d, J = 6.1 Hz, 1H) |
| 14 | 1.32-1.41 (m, 2H), 1.76-1.86 (m, 2H), 2.47-2.49 (m, 2H), 2.76 (t, J = 7.4 Hz, 2H), 2.95 (t, |
| | J = 8.7 Hz, 2H), 3.23-3.26 (m, 2H), 3.26 (s, 3H), 3.93 (d, J = 6.4 Hz, 2H), 4.03 (s, 2H), 4.39 (t, |
| | J = 8.7 Hz, 2H), 5.71 (d, J = 2.8 Hz, 1H), 5.81 (dd, J = 7.5, 2.8 Hz, 1H), 6.21 (t, J = 6.6 Hz, 1H), |
| | 6.48 (s, 2H), 6.56 (d, J = 8.1 Hz, 1H), 6.75 (d, J = 7.8 Hz, 1H), 6.78 (d, J = 6.4 Hz, 1H), 6.91 |
| | (dd, J = 7.8, 1.7 Hz, 1H), 7.03 (s, 1H), 7.17 (t, J = 8.0 Hz, 1H), 7.29 (d, J = 8.3 Hz, 1H), 7.49 (d, |
| | J = 7.5 Hz, 1H), 7.68 (d, J = 6.1 Hz, 1H) |
| 15 | 1.47-1.53 (2H, m), 1.87-1.94 (4H, m), 2.69 (2H, t, J = 6.4 Hz), 3.31 (3H, s), 3.52 (2H, s), |
| | 3.56 (2H, s), 3.98 (2H, d, J = 6.6 Hz), 4.07-4.13 (2H, m), 4.14 (2H, s), 5.78 (1H, d, J = 2.7 |
| | Hz), 5.88 (1H, dd, J = 7.5, 2.8 Hz), 6.16 (1H, t, J = 6.6 Hz), 6.51 (2H, d, J = 5.3 Hz), 6.66 (1H, |
| | d, J = 8.1 Hz), 6.79 (2H, t, J = 7.5 Hz), 6.97 (2H, d, J = 8.9 Hz), 7.21 (1H, t, J = 8.0 Hz), 7.33 |
| | (1H, d, J = 8.3 Hz), 7.54 (1H, d, J = 7.5 Hz), 7.70 (1H, d, J = 6.1 Hz) |
| 16 | 1.01 (1H, dt, J = 12.6, 6.3 Hz), 1.09-1.22 (1H, m), 1.49 (2H, d, J = 5.0 Hz), 1.66-1.78 (2H, |
| | m), 1.89 (2H, d, J = 5.2 Hz), 1.97 (3H, s), 2.01-2.11 (1H, m), 2.21 (2H, d, J = 6.9 Hz), 3.02 |
| | (1H, t, J = 12.5 Hz), 3.29 (1H, s), 3.31 (3H, s), 3.48 (2H, s), 3.77 (1H, d, J = 13.5 Hz), 3.98 (2H, |
| | d, J = 6.5 Hz), 4.13 (2H, s), 4.33 (1H, d, J = 13.6 Hz), 5.77 (1H, d, J = 2.8 Hz), 5.88 (1H, dd, J = |
| | 7.5, 2.7 Hz), 6.21 (1H, t, J = 6.8 Hz), 6.51 (2H, d, J = 5.3 Hz), 6.80 (2H, dd, J = 9.9, 7.0 Hz), |
| | 7.21 (1H, t, J = 8.0 Hz), 7.32 (1H, d, J = 8.3 Hz), 7.53 (1H, d, J = 7.6 Hz), 7.71 (1H, d, J = 6.1 |
| | Hz) |
| 17 | 1.49-1.55 (2H, m), 1.85-1.93 (2H, m), 3.31 (3H, s), 3.57 (3H, d, J = 3.3 Hz), 3.59 (1H, d, J = |
| | 9.7 Hz), 3.98 (2H, d, J = 6.5 Hz), 4.14 (2H, s), 4.54 (2H, d, J = 1.6 Hz), 5.77 (1H, d, J = 2.8 Hz), |
| | 5.88 (1H, dd, J = 7.6, 2.7 Hz), 6.13 (1H, t, J = 6.7 Hz), 6.50 (2H, d, J = 5.3 Hz), 6.78 (2H, d, J = |
| | 8.7 Hz), 6.83 (1H, d, J = 8.3 Hz), 6.86-6.89 (2H, m), 7.20 (1H, t, J = 8.0 Hz), 7.32 (1H, d, J = |
| | 8.3 Hz), 7.53 (1H, d, J = 7.6 Hz), 7.70 (1H, d, J = 6.0 Hz), 10.71 (1H, s) |
| 18 | 1.16 (2H, q, J = 4.6 Hz), 1.40 (4H, q, J = 4.1 Hz), 1.81-1.88 (2H, m), 3.17 (2H, s), 3.28 (3H, |
| | s), 4.01 (2H, s), 4.05 (2H, d, J = 6.6 Hz), 5.68 (1H, d, J = 2.8 Hz), 5.78 (1H, dd, J = 7.5, 2.7 Hz), |
| | 6.14 (1H, t, J = 6.7 Hz), 6.54 (2H, s), 6.81 (1H, d, J = 7.8 Hz), 6.90 (1H, d, J = 6.2 Hz), 7.21 |
| | (4H, ddd, J = 10.7, 5.3, 3.3 Hz), 7.24-7.29 (2H, m), 7.34 (1H, d, J = 8.3 Hz), 7.49 (1H, d, J = |
| | 7.5 Hz), 7.75 (1H, d, J = 6.0 Hz) |
| 19 | 1.47-1.55 (2H, m), 1.85-1.93 (2H, m), 2.58 (2H, t, J = 7.4 Hz), 2.69 (2H, t, J = 7.3 Hz), 3.31 |
| | (3H, s), 3.37 (3H, s), 3.50 (2H, s), 3.97 (2H, d, J = 6.5 Hz), 4.13 (2H, s), 5.77 (1H, d, J = 2.8 |
| | Hz), 5.88 (1H, dd, J = 7.5, 2.8 Hz), 6.13-6.20 (2H, m), 6.26 (1H, d, J = 1.9 Hz), 6.51 (2H, s), |
| | 6.76-6.82 (2H, m), 7.20 (1H, t, J = 8.0 Hz), 7.33 (1H, d, J = 8.3 Hz), 7.53 (1H, d, J = 7.5 Hz), |
| | 7.56 (1H, d, J = 6.9 Hz), 7.71 (1H, d, J = 6.0 Hz) |
| 20 | 1.44-1.47 (2H, m), 1.47-1.54 (2H, m), 1.59 (2H, t, J = 5.2 Hz), 1.83-1.91 (2H, m), 2.00 |
| | (4H, d, J = 8.7 Hz), 2.26 (3H, s), 3.20-3.26 (1H, m), 3.30 (3H, s), 3.38 (2H, s), 3.45 (2H, t, J = |
| | 5.3 Hz), 3.52 (2H, t, J = 5.2 Hz), 3.99 (2H, d, J = 6.5 Hz), 4.09 (2H, s), 5.67 (1H, d, J = 2.8 Hz), |
| | 5.84 (1H, dd, J = 2.8, 0.9 Hz), 6.21 (1H, t, J = 6.6 Hz), 6.52 (2H, s), 6.79 (1H, d, J = 7.8 Hz), |
| | 6.83 (1H, d, J = 6.1 Hz), 7.21 (1H, t, J = 8.0 Hz), 7.33 (1H, d, J = 8.3 Hz), 7.70 (1H, d, J = 6.1 |
| | Hz) |
| 21 | 1.41-1.46 (2H, m), 1.51 (13H, s), 1.83-1.89 (2H, m), 2.00 (2H, s), 2.27 (3H, s), 3.31 (3H, s), |
| | 3.46 (2H, s), 3.97 (2H, d, J = 6.6 Hz), 4.10 (2H, s), 5.67 (1H, d, J = 2.8 Hz), 5.84 (1H, dd, J = |
| | 2.8, 0.9 Hz), 6.16 (1H, t, J = 6.7 Hz), 6.52 (2H, s), 6.76-6.85 (2H, m), 7.21 (1H, t, J = 8.0 Hz), |
| | 7.32 (1H, d, J = 8.3 Hz), 7.71 (1H, d, J = 6.1 Hz) |
| 22 | 1.21-1.28 (2H, m), 1.31 (2H, s), 1.32-1.40 (2H, m), 1.44-1.61 (6H, m), 1.83-1.93 (2H, |
| | m), 2.14 (1H, s), 2.27 (3H, s), 2.46 (2H, s), 3.31 (3H, s), 3.49 (2H, s), 3.97 (2H, d, J = 6.4 Hz), |
| | 4.11 (2H, s), 5.68 (1H, d, J = 2.8 Hz), 5.84 (1H, dd, J = 2.8, 0.9 Hz), 6.21 (1H, t, J = 6.7 Hz), |
| | 6.52 (2H, s), 6.78 (1H, d, J = 6.2 Hz), 6.82 (1H, d, J = 7.7 Hz), 7.21 (1H, t, J = 8.0 Hz), 7.32 |
| | (1H, d, J = 8.3 Hz), 7.69 (1H, d, J = 6.1 Hz) |
| 23 | 1.19-1.31 (2H, m), 1.45-1.52 (2H, m), 1.74-1.86 (4H, m), 1.87-1.91 (2H, m), 1.91-2.03 |
| | (3H, m), 2.23 (2H, d, J = 6.9 Hz), 2.26 (3H, s), 3.31 (3H, s), 3.48 (2H, s), 3.98 (2H, d, J = 6.6 |
| | Hz), 4.10 (2H, s), 5.68 (1H, d, J = 2.7 Hz), 5.83 (1H, dd, J = 2.9, 0.9 Hz), 6.20 (1H, t, J = 6.7 |
| | Hz), 6.52 (2H, s), 6.80 (2H, d, J = 7.1 Hz), 7.20 (1H, t, J = 8.0 Hz), 7.32 (1H, d, J = 8.3 Hz), |
| | 7.71 (1H, d, J = 6.1 Hz) |
| 24 | 0.93-1.17 (4H, m), 1.45-1.50 (2H, m), 1.71-1.81 (3H, m), 1.84-1.90 (2H, m), 1.93-2.01 |
| | (2H, m), 2.15 (2H, d, J = 6.5 Hz), 2.26 (3H, s), 2.99-3.08 (1H, m), 3.22 (3H, s), 3.31 (3H, s), |
| | 3.46 (2H, s), 3.97 (2H, d, J = 6.5 Hz), 4.10 (2H, s), 5.68 (1H, d, J = 2.8 Hz), 5.84 (1H, dd, J = |
| | 2.8, 0.9 Hz), 6.20 (1H, t, J = 6.8 Hz), 6.52 (2H, s), 6.76-6.84 (2H, m), 7.20 (1H, t, J = 8.0 Hz), |
| | 7.32 (1H, d, J = 8.3 Hz), 7.70 (1H, d, J = 6.1 Hz) |
| 25 | 1.22-1.32 (2H, m), 1.37-1.51 (6H, m), 1.73-1.82 (2H, m), 1.83-1.92 (3H, m), 2.15 (2H, d, |
| | J = 7.0 Hz), 2.26 (3H, s), 3.19 (3H, s), 3.31 (3H, s), 3.34-3.38 (1H, m), 3.48 (2H, s), 3.98 (2H, |
| | d, J = 6.6 Hz), 4.10 (2H, s), 5.68 (1H, d, J = 2.8 Hz), 5.84 (1H, dd, J = 2.8, 0.9 Hz), 6.23 (1H, t, |
| | J = 6.7 Hz), 6.60 (2H, s), 6.78-6.86 (2H, m), 7.22 (1H, t, J = 8.0 Hz), 7.33 (1H, d, J = 8.3 Hz), |
| | 7.70 (1H, d, J = 6.0 Hz) |
| 26 | 1.47 (2H, d, J = 4.4 Hz), 1.78 (6H, s), 1.87-1.92 (2H, m), 2.27 (3H, s), 2.43 (2H, s), 2.48 (1H, |
| | s), 3.31 (3H, s), 3.45 (2H, s), 3.96 (2H, d, J = 6.2 Hz), 4.12 (2H, s), 5.68 (1H, d, J = 2.8 Hz), |
| | 5.84 (1H, dd, J = 2.9, 0.9 Hz), 6.23 (1H, t, J = 6.9 Hz), 6.54 (2H, s), 6.82 (2H, d, J = 7.2 Hz), |
| | 7.21 (1H, t, J = 8.0 Hz), 7.32 (1H, d, J = 8.3 Hz), 7.70 (1H, d, J = 6.0 Hz) |
| 27 | 1.13-1.20 (1H, m), 1.31 (3H, d, J = 6.8 Hz), 1.53-1.60 (1H, m), 1.81 (1H, d, J = 7.0 Hz), 1.89 |
| | (1H, d, J = 6.9 Hz), 3.01-3.18 (2H, m), 3.25 (1H, d, J = 7.7 Hz), 3.30 (3H, s), 3.65 (1H, d, J = |
| | 7.7 Hz), 3.79 (1H, q, J = 6.8 Hz), 4.00 (2H, d, J = 6.6 Hz), 4.08 (2H, s), 4.48 (2H, t, J = 8.7 Hz), |
| | 5.73 (1H, d, J = 2.8 Hz), 5.84 (1H, dd, J = 7.5, 2.7 Hz), 6.15 (1H, t, J = 6.7 Hz), 6.50 (2H, s), |
| | 6.66 (1H, d, J = 8.1 Hz), 6.77-6.83 (2H, m), 7.03-7.09 (1H, m), 7.16-7.23 (2H, m), 7.32 |
| | (1H, d, J = 8.3 Hz), 7.52 (1H, d, J = 7.6 Hz), 7.72 (1H, d, J = 6.1 Hz) |
| 28 | 1.14-1.19 (1H, m), 1.31 (3H, d, J = 6.8 Hz), 1.55 (1H, dd, J = 10.1, 7.0 Hz), 1.80 (1H, d, J = |
| | 7.0 Hz), 1.88 (1H, d, J = 6.9 Hz), 2.25 (3H, s), 3.01-3.18 (2H, m), 3.24 (1H, d, J = 7.7 Hz), |
| | 3.30 (3H, s), 3.64 (1H, d, J = 7.7 Hz), 3.79 (1H, q, J = 6.7 Hz), 4.00 (2H, d, J = 6.6 Hz), 4.06 |
| | (2H, s), 4.48 (2H, t, J = 8.7 Hz), 5.64 (1H, d, J = 2.8 Hz), 5.79 (1H, d, J = 2.7 Hz), 6.15 (1H, t, |
| | J = 6.8 Hz), 6.50 (2H, s), 6.66 (1H, d, J = 8.1 Hz), 6.77-6.83 (2H, m), 7.06 (1H, dd, J = 8.2, 2.0 |
| | Hz), 7.16-7.23 (2H, m), 7.32 (1H, d, J = 8.3 Hz), 7.72 (1H, d, J = 6.1 Hz) |
| 29 | 1.27 (3H, d, J = 6.9 Hz), 1.53 (1H, d, J = 3.4 Hz), 1.89 (3H, s), 1.92 (2H, t, J = 5.9 Hz), 2.30 |
| | (3H, s), 3.34 (4H, s), 3.57 (1H, d, J = 7.4 Hz), 3.72 (1H, d, J = 7.5 Hz), 3.94-4.11 (2H, m), |
| | 4.17 (2H, s), 4.47 (1H, t, J = 7.1 Hz), 5.73 (1H, d, J = 2.8 Hz), 5.89 (1H, dd, J = 2.9, 0.9 Hz), |
| | 6.16 (1H, t, J = 6.5 Hz), 6.56 (2H, s), 6.82 (1H, d, J = 7.7 Hz), 6.87 (1H, d, J = 6.2 Hz), 7.25 |
| | (1H, t, J = 8.0 Hz), 7.37 (1H, d, J = 8.0 Hz), 7.74 (1H, d, J = 8.0 Hz) 8.24 (1H, d, J = 8.0 Hz) |
| 30 | 1.13-1.22 (4H, m), 1.45-1.50 (2H, m), 1.78-1.83 (2H, m), 3.31 (3H, s), 3.36 (2H, s), 3.90 |
| | (2H, d, J = 6.4 Hz), 4.15 (2H, s), 5.78 (1H, d, J = 2.8 Hz), 5.89 (1H, dd, J = 7.5, 2.7 Hz), 6.19 |
| | (1H, t, J = 6.5 Hz), 6.55 (2H, s), 6.75 (1H, d, J = 7.7 Hz), 6.79 (1H, d, J = 6.2 Hz), 7.13-7.32 |
| | (8H, m), 7.53 (1H, d, J = 7.6 Hz), 7.64 (1H, d, J = 6.1 Hz) |
| 31 | 1.55 (2H, dd, J = 4.6, 1.7 Hz), 1.74 (2H, d, J = 4.8 Hz), 3.29 (3H, s), 3.36-3.39 (6H, m), 3.57 |
| | (4H, t, J = 4.7 Hz), 3.82 (2H, d, J = 6.1 Hz), 4.10 (2H, s), 5.73 (1H, d, J = 2.8 Hz), 5.87 (1H, dd, |
| | J = 7.5, 2.8 Hz), 6.18 (1H, t, J = 6.2 Hz), 6.48 (2H, s), 6.71 (1H, d, J = 7.7 Hz), 7.08 (1H, d, J = |
| | 6.2 Hz), 7.19 (1H, t, J = 8.0 Hz), 7.29 (1H, d, J = 8.3 Hz), 7.51 (1H, d, J = 7.6 Hz), 7.71 (1H, d, |
| | J = 6.1 Hz) |
| 32 | 1.38 (3H, d, J = 7.1 Hz), 1.40-1.46 (1H, m), 1.47-1.54 (1H, m), 1.79 (2H, dd, J = 10.3, 6.7 |
| | Hz), 3.31 (3H, s), 3.40 (2H, s), 3.89 (2H, d, J = 6.4 Hz), 4.15 (2H, s), 4.88 (1H, p, J = 7.1 Hz), |
| | 5.78 (1H, d, J = 2.8 Hz), 5.89 (1H, dd, J = 7.5, 2.7 Hz), 6.23 (1H, t, J = 6.5 Hz), 6.49 (2H, s), |
| | 6.73 (2H, dd, J = 7.8, 6.6 Hz), 6.80 (1H, d, J = 6.1 Hz), 7.15-7.25 (2H, m), 7.27-7.34 (3H, |
| | m), 7.34-7.39 (2H, m), 7.53 (1H, d, J = 7.5 Hz), 7.64 (1H, d, J = 6.0 Hz) |
| 33 | 1.38 (3H, d, J = 7.1 Hz), 1.43 (1H, dd, J = 10.0, 6.7 Hz), 1.51 (1H, dd, J = 10.0, 6.6 Hz), 1.79 |
| | (2H, dd, J = 10.3, 6.7 Hz), 3.31 (3H, s), 3.40 (2H, s), 3.89 (2H, d, J = 6.3 Hz), 4.15 (2H, s), 4.88 |
| | (1H, p, J = 7.2 Hz), 5.78 (1H, d, J = 2.8 Hz), 5.89 (1H, dd, J = 7.5, 2.8 Hz), 6.23 (1H, t, J = 6.5 |
| | Hz), 6.51 (2H, s), 6.73 (2H, dd, J = 8.0, 5.4 Hz), 6.80 (1H, d, J = 6.2 Hz), 7.15-7.24 (2H, m), |
| | 7.28-7.34 (3H, m), 7.34-7.38 (2H, m), 7.53 (1H, d, J = 7.6 Hz), 7.64 (1H, d, J = 6.0 Hz) |
| 34 | 1.61-1.70 (2H, m), 1.82 (2H, d, J = 4.7 Hz), 3.09 (2H, t, J = 8.4 Hz), 3.28 (3H, s), 3.37 (2H, s), |
| | 3.91 (2H, d, J = 5.9 Hz), 4.01 (2H, t, J = 8.4 Hz), 4.12 (2H, s), 5.73 (1H, d, J = 2.8 Hz), 5.86 |
| | (1H, dd, J = 7.5, 2.8 Hz), 6.20 (1H, t, J = 6.2 Hz), 6.47 (2H, d, J = 4.5 Hz), 6.75 (1H, d, J = 7.7 |
| | Hz), 6.91 (1H, td, J = 7.4, 1.1 Hz), 7.11 (1H, d, J = 6.2 Hz), 7.15 (1H, t, J = 7.0 Hz), 7.21 (2H, |
| | dt, J = 8.1, 4.3 Hz), 7.31 (1H, d, J = 8.3 Hz), 7.49 (1H, d, J = 7.6 Hz), 7.60 (1H, d, J = 8.2 Hz), |
| | 7.72 (1H, d, J = 6.1 Hz) |
| 35 | 1.60-1.64 (2H, m), 1.83 (2H, d, J = 4.7 Hz), 3.26 (2H, s), 3.28 (3H, s), 3.73-3.86 (2H, m), |
| | 3.97 (2H, d, J = 5.9 Hz), 4.07 (2H, s), 4.22-4.31 (2H, m), 5.71 (1H, d, J = 2.8 Hz), 5.83 (1H, |
| | dd, J = 7.6, 2.8 Hz), 6.16 (1H, t, J = 6.3 Hz), 6.48 (2H, d, J = 4.4 Hz), 6.75 (1H, d, J = 7.7 Hz), |
| | 6.82-6.89 (2H, m), 6.89-6.96 (1H, m), 7.11 (1H, d, J = 6.1 Hz), 7.22 (1H, t, J = 8.0 Hz), 7.32 |
| | (1H, d, J = 8.3 Hz), 7.48 (1H, d, J = 7.6 Hz), 7.57 (1H, dd, J = 8.4, 1.7 Hz), 7.72 (1H, d, J = 6.1 |
| | Hz) |
| 36 | 1.56 (2H, dd, J = 4.6, 1.8 Hz), 1.78-1.82 (2H, m), 1.89 (2H, p, J = 6.5 Hz), 2.72 (2H, t, J = 6.6 |
| | Hz), 3.03 (2H, s), 3.27 (3H, s), 3.63 (2H, dd, J = 6.9, 5.4 Hz), 3.98 (2H, d, J = 6.1 Hz), 4.03 |
| | (2H, s), 5.68 (1H, d, J = 2.8 Hz), 5.81 (1H, dd, J = 7.6, 2.8 Hz), 6.16 (1H, t, J = 6.3 Hz), 6.48 |
| | (2H, s), 6.75 (1H, d, J = 7.7 Hz), 6.93 (1H, td, J = 7.4, 1.2 Hz), 7.06-7.14 (3H, m), 7.22 (1H, t, |
| | J = 8.0 Hz), 7.32 (1H, d, J = 8.3 Hz), 7.43 (1H, dd, J = 8.1, 1.3 Hz), 7.47 (1H, d, J = 7.6 Hz), |
| | 7.72 (1H, d, J = 6.1 Hz) |
| 37 | 1.59 (2H, dd, J = 4.6, 1.8 Hz), 1.73-1.79 (2H, m), 2.84 (2H, t, J = 5.8 Hz), 3.29 (3H, s), 3.37 |
| | (2H, s), 3.63 (2H, t, J = 5.9 Hz), 3.83 (2H, d, J = 6.0 Hz), 4.11 (2H, s), 4.57 (2H, s), 5.73 (1H, d, |
| | J = 2.8 Hz), 5.87 (1H, dd, J = 7.5, 2.8 Hz), 6.19 (1H, t, J = 6.2 Hz), 6.46 (2H, s), 6.72 (1H, d, J = |
| | 7.7 Hz), 7.07 (1H, d, J = 6.2 Hz), 7.12-7.22 (5H, m), 7.28 (1H, d, J = 8.3 Hz), 7.50 (1H, d, J = |
| | 7.6 Hz), 7.68 (1H, d, J = 6.0 Hz) |
| 38 | 1.56 (2H, d, J = 4.2 Hz), 1.84 (2H, d, J = 4.6 Hz), 3.14 (2H, t, J = 8.7 Hz), 3.30 (3H, s), 3.49 |
| | (2H, s), 3.99 (3H, d, J = 6.2 Hz), 4.16 (2H, s), 4.48 (2H, t, J = 8.7 Hz), 5.78 (1H, d, J = 2.8 Hz), |
| | 5.89 (1H, dd, J = 7.5, 2.8 Hz), 6.21 (1H, t, J = 6.5 Hz), 6.52 (2H, s), 6.64 (1H, d, J = 8.5 Hz), |
| | 6.75 (1H, d, J = 7.7 Hz), 6.97 (1H, d, J = 6.2 Hz), 7.11 (1H, dd, J = 8.5, 2.3 Hz), 7.21 (1H, t, J = |
| | 8.0 Hz), 7.32 (1H, d, J = 8.3 Hz), 7.39 (1H, d, J = 2.2 Hz), 7.53 (1H, d, J = 7.6 Hz), 7.71 (1H, d, |
| | J = 6.1 Hz), 8.31 (1H, s) |
| 39 | 1.53 (2H, dd, J = 4.4, 1.8 Hz), 1.75-1.81 (2H, m), 1.86 (2H, p, J = 6.5 Hz), 2.70 (2H, t, J = 6.7 |
| | Hz), 2.97 (2H, s), 3.28 (3H, d, J = 5.6 Hz), 3.60 (2H, t, J = 6.2 Hz), 3.71 (3H, s), 3.96 (2H, d, J = |
| | 5.9 Hz), 4.02 (2H, s), 5.68 (1H, d, J = 2.8 Hz), 5.81 (1H, dd, J = 7.5, 2.8 Hz), 6.18 (1H, t, J = |
| | 6.4 Hz), 6.53 (2H, s), 6.71 (2H, dq, J = 5.9, 3.1 Hz), 6.76 (1H, d, J = 7.7 Hz), 7.11 (1H, d, J = |
| | 6.2 Hz), 7.22 (1H, t, J = 8.0 Hz), 7.34 (2H, dd, J = 14.8, 9.0 Hz), 7.47 (1H, d, J = 7.6 Hz), 7.72 |
| | (1H, d, J = 6.1 Hz) |
| 40 | 1.54 (2H, d, J = 5.8 Hz), 1.73 (2H, d, J = 4.5 Hz), 1.81-1.92 (1H, m), 2.07-2.21 (1H, m), 2.24 |
| | (3H, s), 2.81 (3H, s), 3.25 (2H, s), 3.29 (3H, s), 3.58 (1H, q, J = 7.9 Hz), 3.64-3.72 (2H, m), |
| | 3.80 (2H, d, J = 6.1 Hz), 3.92 (1H, td, J = 8.4, 4.7 Hz), 4.08 (2H, s), 4.79 (1H, dt, J = 14.1, 5.4 |
| | Hz), 5.64 (1H, d, J = 2.8 Hz), 5.82 (1H, dd, J = 2.8, 0.9 Hz), 6.18 (1H, t, J = 6.3 Hz), 6.46 (2H, |
| | d, J = 4.4 Hz), 6.72 (1H, d, J = 7.7 Hz), 7.08 (1H, d, J = 6.1 Hz), 7.19 (1H, t, J = 8.0 Hz), 7.29 |
| | (1H, d, J = 8.3 Hz), 7.71 (1H, d, J = 6.1 Hz) |
| 41 | 1.55 (2H, d, J = 5.1 Hz), 1.72 (2H, d, J = 3.3 Hz), 1.81-1.92 (1H, m), 2.14 (1H, ddt, J = 13.1, |
| | 8.3, 4.2 Hz), 2.24 (3H, s), 2.81 (3H, s), 3.25 (2H, s), 3.29 (3H, s), 3.58 (1H, q, J = 8.0 Hz), 3.63- |
| | 3.73 (2H, m), 3.80 (2H, d, J = 6.0 Hz), 3.92 (1H, td, J = 8.4, 4.7 Hz), 4.08 (2H, s), 4.79 (1H, |
| | dq, J = 11.6, 5.3 Hz), 5.64 (1H, d, J = 2.8 Hz), 5.82 (1H, dd, J = 2.9, 0.9 Hz), 6.18 (1H, t, J = 6.2 |
| | Hz), 6.46 (2H, s), 6.72 (1H, d, J = 7.7 Hz), 7.08 (1H, d, J = 6.1 Hz), 7.19 (1H, t, J = 8.0 Hz), |
| | 7.29 (1H, d, J = 8.3 Hz), 7.71 (1H, d, J = 6.1 Hz) |
| 42 | 1.46 (3H, d, J = 7.1 Hz), 1.55-1.63 (2H, m), 1.72 (2H, s), 2.54 (3H, s), 2.50-2.60 (5H, m), |
| | 3.81 (2H, d, J = 6.0 Hz), 4.08 (2H, s), 5.43 (1H, q, J = 7.0 Hz), 5.70 (1H, d, J = 2.7 Hz), 5.83 |
| | (1H, dd, J = 7.5, 2.8 Hz), 6.09 (1H, t, J = 6.2 Hz), 6.42 (2H, s), 6.70 (1H, d, J = 7.7 Hz), 7.04 |
| | (1H, d, J = 6.2 Hz), 7.13 (1H, t, J = 8.0 Hz), 7.17-7.32 (6H, m), 7.47 (1H, d, J = 7.6 Hz), 7.66 |
| | (1H, d, J = 6.1 Hz) |
| 43 | 1.46 (3H, d, J = 7.1 Hz), 1.57 (2H, d, J = 4.3 Hz), 1.72 (2H, s), 2.54 (3H, s), 3.20-3.27 (5H, m), |
| | 3.81 (2H, d, J = 6.0 Hz), 4.08 (2H, s), 5.43 (1H, d, J = 7.0 Hz), 5.70 (1H, d, J = 2.8 Hz), 5.83 |
| | (1H, dd, J = 7.5, 2.8 Hz), 6.04-6.13 (1H, m), 6.42 (2H, s), 6.70 (1H, d, J = 7.7 Hz), 7.04 (1H, |
| | d, J = 6.2 Hz), 7.13 (1H, t, J = 8.0 Hz), 7.19-7.35 (6H, m), 7.46 (1H, s), 7.66 (1H, d, J = 6.1 |
| | Hz) |
| 44 | 1.55 (2H, t, J = 6.4 Hz), 1.72 (1H, d, J = 6.0 Hz), 1.77 (1H, d, J = 5.9 Hz), 1.97 (1H, p, J = 10.1 |
| | Hz), 2.20-2.26 (1H, m), 3.21 (1H, dd, J = 7.5, 1.8 Hz), 3.27-3.32 (4H, m), 3.32-3.42 (2H, |
| | m), 3.46 (1H, td, J = 10.2, 6.7 Hz), 3.50-3.57 (1H, m), 3.71 (1H, dd, J = 14.6, 6.1 Hz), 3.79 |
| | (1H, dd, J = 9.8, 7.1 Hz), 4.04 (1H, dd, J = 14.5, 5.9 Hz), 4.10 (2H, s), 5.73 (1H, d, J = 2.8 Hz), |
| | 5.87 (1H, dd, J = 7.6, 2.8 Hz), 6.27 (1H, t, J = 6.3 Hz), 6.52 (2H, s), 6.74 (1H, d, J = 7.8 Hz), |
| | 7.09 (1H, d, J = 6.2 Hz), 7.17-7.28 (2H, m), 7.28-7.36 (5H, m), 7.50 (1H, d, J = 7.6 Hz), 7.71 |
| | (1H, d, J = 6.1 Hz) |
| 45 | 1.48-1.59 (2H, m), 1.71 (1H, d, J = 6.1 Hz), 1.74-1.79 (1H, m), 1.97 (1H, p, J = 10.2 Hz), |
| | 2.19-2.28 (4H, m), 3.20 (1H, d, J = 7.3 Hz), 3.28-3.30 (4H, m), 3.34-3.41 (2H, m), 3.43- |
| | 3.49 (1H, m), 3.50-3.57 (1H, m), 3.71 (1H, dd, J = 14.6, 6.1 Hz), 3.79 (1H, dd, J = 9.9, 7.1 |
| | Hz), 4.02-4.07 (1H, m), 4.08 (2H, s), 5.64 (1H, d, J = 2.8 Hz), 5.82 (1H, dd, J = 2.8, 0.9 Hz), |
| | 6.27 (1H, t, J = 6.2 Hz), 6.49 (2H, s), 6.74 (1H, d, J = 7.8 Hz), 7.09 (1H, d, J = 6.2 Hz), 7.20 |
| | (1H, t, J = 8.1 Hz), 7.22-7.27 (1H, m), 7.28-7.35 (5H, m), 7.71 (1H, d, J = 6.0 Hz) |
| 46 | 1.46 (2H, dd, J = 4.5, 1.8 Hz), 1.68 (4H, t, J = 5.2 Hz), 1.76 (2H, d, J = 6.2 Hz), 2.25 (3H, s), |
| | 3.26 (2H, s), 3.29 (3H, s), 3.51 (4H, t, J = 5.2 Hz), 3.67 (4H, s), 3.93 (2H, d, J = 6.2 Hz), 4.08 |
| | (2H, s), 5.64 (1H, d, J = 2.8 Hz), 5.83 (1H, dd, J = 2.8, 0.9 Hz), 6.28 (1H, t, J = 6.4 Hz), 6.48 |
| | (2H, s), 6.69 (1H, d, J = 7.7 Hz), 7.01 (1H, d, J = 6.1 Hz), 7.19 (1H, t, J = 8.0 Hz), 7.30 (1H, d, |
| | J = 8.3 Hz), 7.71 (1H, d, J = 6.1 Hz) |
| 47 | 1.48-1.60 (3H, m), 1.68-1.78 (2H, m), 1.92 (1H, dtd, J = 13.1, 7.8, 5.3 Hz), 2.24 (3H, s), |
| | 2.51-2.59 (1H, m), 2.93 (3H, s), 3.22 (2H, s), 3.29 (3H, s), 3.35-3.41 (2H, m), 3.62 (1H, q, J = |
| | 7.6 Hz), 3.67-3.77 (2H, m), 3.78 (2H, d, J = 5.4 Hz), 4.09 (2H, s), 5.65 (1H, d, J = 2.8 Hz), |
| | 5.82 (1H, d, J = 2.8 Hz), 6.17 (1H, t, J = 6.2 Hz), 6.47 (2H, s), 6.71 (1H, d, J = 7.6 Hz), 7.08 |
| | (1H, d, J = 6.2 Hz), 7.19 (1H, t, J = 8.0 Hz), 7.29 (1H, d, J = 8.3 Hz), 7.71 (1H, d, J = 6.1 Hz) |
| 48 | 1.48-1.60 (3H, m), 1.67-1.78 (2H, m), 1.87-1.98 (1H, m), 2.24 (3H, s), 2.52-2.59 (1H, |
| | m), 2.93 (3H, s), 3.22 (2H, s), 3.29 (3H, s), 3.38 (2H, dd, J = 8.5, 5.8 Hz), 3.62 (1H, q, J = 7.6 |
| | Hz), 3.70 (1H, dd, J = 8.4, 6.8 Hz), 3.73-3.77 (1H, m), 3.78 (2H, d, J = 5.8 Hz), 4.09 (2H, s), |
| | 5.65 (1H, d, J = 2.8 Hz), 5.82 (1H, dd, J = 2.8, 0.9 Hz), 6.17 (1H, t, J = 6.2 Hz), 6.47 (2H, s), |
| | 6.71 (1H, d, J = 7.7 Hz), 7.08 (1H, d, J = 6.2 Hz), 7.19 (1H, t, J = 8.0 Hz), 7.29 (1H, d, J = 8.3 |
| | Hz), 7.71 (1H, d, J = 6.1 Hz) |
| 49 | 1.49-1.59 (2H, m), 1.71 (1H, d, J = 5.8 Hz), 1.77 (1H, d, J = 5.6 Hz), 1.97 (1H, p, J = 10.0 Hz), |
| | 2.19-2.28 (4H, m), 3.20 (1H, d, J = 7.3 Hz), 3.27-3.30 (4H, m), 3.34-3.58 (4H, m), 3.71 |
| | (1H, dd, J = 14.6, 5.9 Hz), 3.79 (1H, dd, J = 9.6, 6.9 Hz), 4.00-4.07 (1H, m), 4.08 (2H, s), |
| | 5.64 (1H, d, J = 2.8 Hz), 5.82 (1H, dd, J = 2.8, 0.9 Hz), 6.26 (1H, t, J = 6.3 Hz), 6.47 (2H, s), |
| | 6.73 (1H, d, J = 7.7 Hz), 7.09 (1H, d, J = 6.2 Hz), 7.17-7.27 (2H, m), 7.28-7.35 (5H, m), 7.71 |
| | (1H, d, J = 6.1 Hz) |
| 50 | 1.57-1.63 (2H, m), 1.76-1.82 (2H, m), 3.29 (3H, s), 3.42 (2H, s), 3.88 (2H, d, J = 6.0 Hz), |
| | 4.14 (2H, s), 4.74 (4H, s), 5.73-5.77 (1H, m), 5.88 (1H, dd, J = 7.5, 2.8 Hz), 6.25 (1H, t, J = |
| | 6.3 Hz), 6.48 (2H, s), 6.74 (1H, d, J = 7.8 Hz), 7.09 (1H, d, J = 6.1 Hz), 7.19 (1H, t, J = 8.0 Hz), |
| | 7.27-7.32 (3H, m), 7.35 (2H, dd, J = 5.4, 3.3 Hz), 7.51 (1H, d, J = 7.6 Hz), 7.70 (1H, d, J = 6.0 |
| | Hz) |
| 51 | 1.42-1.50 (2H, m), 1.71-1.83 (4H, m), 2.13 (4H, t, J = 7.6 Hz), 3.23 (2H, s), 3.30 (3H, s), |
| | 3.87 (4H, s), 3.92 (2H, d, J = 6.0 Hz), 4.09 (2H, s), 5.73 (1H, d, J = 2.7 Hz), 5.87 (1H, dd, J = |
| | 7.6, 2.7 Hz), 6.27 (1H, t, J = 6.4 Hz), 6.50 (2H, s), 6.69 (1H, d, J = 7.7 Hz), 7.01 (1H, d, J = 6.2 |
| | Hz), 7.19 (1H, t, J = 8.0 Hz), 7.31 (1H, d, J = 8.3 Hz), 7.51 (1H, d, J = 7.6 Hz), 7.71 (1H, d, J = |
| | 6.1 Hz) |
| 52 | 1.55 (2H, d, J = 4.3 Hz), 1.79-1.95 (4H, m), 2.69 (2H, t, J = 6.5 Hz), 3.30 (3H, s), 3.48 (2H, s), |
| | 3.98 (2H, d, J = 6.2 Hz), 4.04-4.11 (2H, m), 4.16 (2H, s), 5.78 (1H, d, J = 2.8 Hz), 5.89 (1H, |
| | dd, J = 7.5, 2.8 Hz), 6.21 (1H, t, J = 6.3 Hz), 6.50 (2H, s), 6.61 (1H, d, J = 8.7 Hz), 6.74 (1H, d, |
| | J = 7.7 Hz), 6.97 (1H, d, J = 6.2 Hz), 7.12 (1H, dd, J = 8.8, 2.6 Hz), 7.17-7.26 (2H, m), 7.33 |
| | (1H, d, J = 8.3 Hz), 7.53 (1H, d, J = 7.6 Hz), 7.71 (1H, d, J = 6.1 Hz), 8.27 (1H, s) |
| 53 | 0.19-0.25 (2H, m), 0.44-0.50 (2H, m), 0.90-1.04 (1H, m), 1.53-1.61 (2H, m), 1.70-1.76 |
| | (2H, m), 2.95 (3H, s), 3.16 (2H, d, J = 6.8 Hz), 3.25 (2H, s), 3.29 (3H, s), 3.78 (2H, d, J = 6.0 |
| | Hz), 4.11 (2H, s), 5.73 (1H, d, J = 2.8 Hz), 5.87 (1H, dd, J = 7.5, 2.8 Hz), 6.18 (1H, t, J = 6.2 |
| | Hz), 6.46 (2H, s), 6.72 (1H, d, J = 7.7 Hz), 7.08 (1H, dd, J = 6.3, 0.8 Hz), 7.19 (1H, t, J = 8.0 |
| | Hz), 7.29 (1H, d, J = 8.3 Hz), 7.50 (1H, d, J = 7.6 Hz), 7.71 (1H, d, J = 6.1 Hz) |
| 54 | 1.48 (4H, d, J = 6.2 Hz), 1.56 (4H, dd, J = 4.4, 1.8 Hz), 1.73 (2H, d, J = 4.2 Hz), 3.26 (2H, s), |
| | 3.29 (3H, s), 3.35 (4H, t, J = 5.6 Hz), 3.79 (2H, d, J = 5.9 Hz), 4.10 (2H, s), 5.73 (1H, d, J = 2.8 |
| | Hz), 5.87 (1H, dd, J = 7.5, 2.8 Hz), 6.17 (1H, t, J = 6.2 Hz), 6.46 (2H, d, J = 4.5 Hz), 6.68-6.74 |
| | (1H, m), 7.07 (1H, dd, J = 6.3, 0.8 Hz), 7.19 (1H, t, J = 8.0 Hz), 7.29 (1H, d, J = 8.3 Hz), 7.50 |
| | (1H, d, J = 7.6 Hz), 7.71 (1H, d, J = 6.1 Hz) |
| 55 | 1.04-1.22 (4H, m), 1.44 (2H, d, J = 4.0 Hz), 1.78 (2H, d, J = 4.5 Hz), 2.28 (3H, s), 3.31 (3H, |
| | s), 3.36 (2H, s), 3.78 (2H, d, J = 6.3 Hz), 4.14 (2H, s), 5.71 (1H, d, J = 2.8 Hz), 5.81 (1H, t, J = |
| | 6.5 Hz), 5.87 (1H, dd, J = 2.9, 0.9 Hz), 6.91 (1H, d, J = 9.6 Hz), 6.98-7.06 (1H, m), 7.10- |
| | 7.30 (6H, m), 7.72 (1H, d, J = 4.5 Hz), 7.95 (1H, d, J = 2.8 Hz) |
| 56 | 1.16 (4H, d, J = 9.3 Hz), 1.43 (2H, d, J = 4.0 Hz), 1.76 (2H, d, J = 4.2 Hz), 2.14 (3H, s), 2.28 |
| | (3H, s), 3.30-3.32 (5H, m), 3.78 (2H, d, J = 6.5 Hz), 4.12 (2H, s), 5.40 (1H, t, J = 6.6 Hz), 5.70 |
| | (1H, d, J = 2.8 Hz), 5.84-5.88 (1H, m), 6.91-7.00 (2H, m), 7.10-7.29 (6H, m), 7.62 (1H, dd, |
| | J = 4.3, 1.6 Hz) |
| 57 | 1.12-1.22 (4H, m), 1.47 (2H, d, J = 4.3 Hz), 1.79 (2H, d, J = 4.4 Hz), 2.26 (3H, s), 3.32 (3H, |
| | s), 3.36 (2H, s), 3.91 (2H, d, J = 5.9 Hz), 4.12 (2H, s), 5.69 (1H, d, J = 2.8 Hz), 5.84 (1H, d, J = |
| | 2.7 Hz), 6.25 (1H, t, J = 6.5 Hz), 6.72-6.80 (2H, m), 6.83 (1H, d, J = 6.3 Hz), 7.10-7.38 (9H, |
| | m), 7.63 (1H, d, J = 6.2 Hz) |
| 58 | 1.06-1.19 (4H, m), 1.44 (2H, d, J = 3.8 Hz), 1.77 (2H, d, J = 4.6 Hz), 2.28 (3H, s), 3.31-3.32 |
| | (5H, m), 3.58 (2H, s), 4.14 (2H, s), 5.03 (1H, s), 5.72 (1H, d, J = 2.8 Hz), 5.88 (1H, d, J = 2.7 |
| | Hz), 6.31 (1H, d, J = 8.6 Hz), 6.86 (1H, dd, J = 8.7, 3.0 Hz), 7.09-7.17 (3H, m), 7.22-7.31 |
| | (3H, m), 7.38 (1H, d, J = 2.8 Hz) |
| 59 | 1.12-1.24 (4H, m), 1.41-1.53 (2H, m), 1.76-1.84 (2H, m), 1.97-2.10 (2H, m), 2.95 (2H, t, |
| | J = 7.7 Hz), 3.36 (2H, s), 3.79-3.86 (2H, m), 3.90 (2H, d, J = 6.4 Hz), 4.13 (2H, s), 5.61 (1H, |
| | d, J = 2.5 Hz), 5.80-5.89 (1H, m), 6.18 (1H, t, J = 6.5 Hz), 6.51 (2H, s), 6.73 (1H, d, J = 7.7 |
| | Hz), 6.78 (1H, d, J = 6.0 Hz), 7.13-7.22 (4H, m), 7.23-7.32 (4H, m), 7.64 (1H, d, J = 6.1 Hz) |
| 60 | 1.10 (1H, d, J = 7.3 Hz), 1.21 (1H, dd, J = 9.5, 4.8 Hz), 1.49 (2H, s), 1.80 (2H, s), 1.92 (1H, s), |
| | 2.73 (1H, s), 3.31-3.33 (5H, m), 3.92 (2H, s), 4.13 (2H, s), 5.77 (1H, d, J = 2.4 Hz), 5.84- |
| | 5.92 (1H, m), 6.34 (1H, s), 6.49 (2H, s), 6.74 (2H, d, J = 7.4 Hz), 6.93 (1H, d, J = 6.4 Hz), 7.09- |
| | 7.34 (7H, m), 7.53 (1H, d, J = 7.5 Hz), 7.70 (1H, d, J = 6.0 Hz) |
| 61 | 1.42-1.49 (2H, m), 1.62 (6H, s), 1.74-1.79 (2H, m), 1.93-2.04 (9H, m), 3.30 (5H, s), 3.89 |
| | (2H, d, J = 6.0 Hz), 4.10 (2H, s), 5.60 (1H, s), 5.75 (1H, d, J = 2.6 Hz), 5.88 (1H, dd, J = 7.5, 2.6 |
| | Hz), 6.21 (1H, t, J = 6.4 Hz), 6.49 (2H, s), 6.73 (1H, d, J = 7.7 Hz), 6.95 (1H, d, J = 6.2 Hz), |
| | 7.20 (1H, t, J = 7.9 Hz), 7.31 (1H, d, J = 8.3 Hz), 7.52 (1H, d, J = 7.5 Hz), 7.70 (1H, d, J = 6.1 |
| | Hz) |
| 62 | 1.68 (2H, d, J = 3.4 Hz), 1.90 (2H, d, J = 3.0 Hz), 3.29 (3H, s), 3.55 (2H, s), 3.96 (2H, d, J = 5.4 |
| | Hz), 4.18 (2H, s), 4.80 (2H, s), 4.92 (2H, s), 5.79 (1H, d, J = 2.6 Hz), 5.91 (1H, dd, J = 7.6, 2.8 |
| | Hz), 5.99-6.08 (1H, m), 6.53 (2H, s), 6.54-6.60 (1H, m), 6.77 (1H, d, J = 7.7 Hz), 7.03 (1H, |
| | d, J = 6.2 Hz), 7.24 (1H, t, J = 8.0 Hz), 7.39 (1H, d, J = 8.4 Hz), 7.54 (1H, d, J = 7.5 Hz), 7.61- |
| | 7.71 (1H, m), 7.73 (1H, d, J = 6.0 Hz), 7.89 (1H, s) |
| 63 | 1.45-1.52 (2H, m), 1.77-1.85 (2H, m), 3.30 (2H, s), 3.30 (3H, s), 3.89 (2H, d, J = 6.2 Hz), |
| | 4.13 (2H, s), 5.77 (1H, d, J = 2.7 Hz), 5.89 (1H, dd, J = 7.5, 2.7 Hz), 5.92 (2H, s), 6.50-6.59 |
| | (3H, m), 6.77 (1H, d, J = 7.7 Hz), 6.88 (1H, d, J = 6.2 Hz), 7.20 (1H, t, J = 8.0 Hz), 7.31 (1H, d, |
| | J = 8.3 Hz), 7.53 (1H, d, J = 7.5 Hz), 7.70 (1H, d, J = 6.1 Hz) |
| 64 | 1.48 (2H, dd, J = 4.3, 1.7 Hz), 1.75-1.83 (2H, m), 1.89 (2H, dt, J = 13.6, 9.0 Hz), 2.37 (2H, d, |
| | J = 13.3 Hz), 3.31 (3H, s), 3.54 (2H, s), 3.71 (4H, d, J = 8.1 Hz), 3.82 (2H, d, J = 6.2 Hz), 4.18 |
| | (2H, s), 5.80 (1H, d, J = 2.8 Hz), 5.90 (1H, dd, J = 7.5, 2.8 Hz), 5.95 (1H, d, J = 6.9 Hz), 6.44 |
| | (1H, s), 6.55 (2H, s), 6.66 (1H, d, J = 6.2 Hz), 6.71 (1H, d, J = 7.8 Hz), 7.13-7.26 (2H, m), |
| | 7.27-7.36 (3H, m), 7.39-7.47 (2H, m), 7.54 (1H, d, J = 7.5 Hz), 7.65 (1H, d, J = 6.1 Hz) |
| 65 | 1.52-1.58 (2H, m), 1.84 (2H, d, J = 4.6 Hz), 1.99 (2H, p, J = 7.4 Hz), 2.26 (3H, s), 2.80 (4H, |
| | dt, J = 11.3, 7.4 Hz), 3.30 (3H, s), 3.50 (2H, s), 3.99 (2H, d, J = 6.2 Hz), 4.14 (2H, s), 5.69 (1H, |
| | d, J = 2.8 Hz), 5.84 (1H, dd, J = 2.8, 0.9 Hz), 6.20 (1H, t, J = 6.4 Hz), 6.50 (2H, s), 6.74 (1H, d, |
| | J = 7.7 Hz), 6.98 (1H, d, J = 6.2 Hz), 7.07 (1H, d, J = 8.1 Hz), 7.17-7.26 (2H, m), 7.33 (1H, d, |
| | J = 8.3 Hz), 7.41 (1H, s), 7.71 (1H, d, J = 6.0 Hz), 8.40 (1H, s) |
| 66 | 1.44-1.53 (2H, m), 1.56 (6H, s), 1.76-1.81 (2H, m), 3.31 (3H, s), 3.46 (2H, s), 3.81 (2H, d, J = |
| | 6.3 Hz), 4.16 (2H, s), 5.79 (1H, d, J = 2.8 Hz), 5.91 (1H, dd, J = 7.5, 2.7 Hz), 6.04 (1H, t, J = |
| | 6.4 Hz), 6.42 (1H, s), 6.47 (2H, s), 6.63-6.72 (2H, m), 7.13-7.22 (2H, m), 7.25-7.32 (3H, |
| | m), 7.35-7.41 (2H, m), 7.54 (1H, d, J = 7.6 Hz), 7.64 (1H, d, J = 6.1 Hz) |
| 67 | 1.10-1.21 (4H, m), 1.44 (2H, d, J = 4.0 Hz), 1.77 (2H, d, J = 4.5 Hz), 2.28 (3H, s), 3.29-3.32 |
| | (4H, m), 3.35 (1H, s), 3.82 (2H, d, J = 6.6 Hz), 4.13 (2H, s), 5.71 (1H, d, J = 2.8 Hz), 5.81 (1H, |
| | t, J = 6.7 Hz), 5.86 (1H, dd, J = 2.8, 0.9 Hz), 7.04 (1H, dd, J = 7.5, 5.0 Hz), 7.10-7.31 (8H, m) |
| 68 | 1.09-1.15 (2H, m), 1.19-1.24 (2H, m), 1.39-1.50 (2H, m), 1.72-1.83 (2H, m), 2.28 (3H, |
| | s), 3.31-3.32 (5H, m), 3.91 (2H, d, J = 6.4 Hz), 4.14 (2H, s), 5.71 (1H, d, J = 2.8 Hz), 5.87 |
| | (1H, d, J = 2.7 Hz), 6.17 (1H, t, J = 6.6 Hz), 6.60 (1H, t, J = 7.5 Hz), 6.90 (1H, d, J = 8.6 Hz), |
| | 7.09-7.45 (8H, m) |
| 69 | 1.60 (2H, t, J = 4.7 Hz), 1.87 (2H, d, J = 5.6 Hz), 3.30 (3H, s), 3.37 (2H, s), 3.46 (1H, dd, J = |
| | 10.6, 5.2 Hz), 3.54 (1H, dd, J = 10.7, 6.3 Hz), 4.02 (1H, dd, J = 14.8, 5.7 Hz), 4.10-4.21 (4H, |
| | m), 4.64 (1H, d, J = 7.9 Hz), 4.94 (1H, t, J = 5.7 Hz), 5.77 (1H, d, J = 2.7 Hz), 5.89 (1H, dd, J = |
| | 7.5, 2.7 Hz), 6.45-6.55 (3H, m), 6.82 (1H, d, J = 7.7 Hz), 7.00 (1H, d, J = 6.2 Hz), 7.17-7.28 |
| | (2H, m), 7.25-7.34 (3H, m), 7.41 (2H, d, J = 6.9 Hz), 7.52 (1H, d, J = 7.6 Hz), 7.61 (1H, d, J = |
| | 6.1 Hz) |
| 70 | 1.49-1.65 (4H, m), 1.71-1.83 (4H, m), 2.24 (3H, s), 2.66-2.78 (1H, m), 2.89 (2H, t, J = |
| | 13.0 Hz), 3.29 (3H, s), 3.83 (2H, d, J = 6.0 Hz), 4.09 (2H, s), 4.13 (2H, d, J = 13.2 Hz), 5.66 |
| | (1H, d, J = 2.8 Hz), 5.84 (1H, dd, J = 2.8, 0.9 Hz), 6.17 (1H, t, J = 6.2 Hz), 6.48 (2H, s), 6.74 |
| | (1H, d, J = 7.7 Hz), 7.09 (1H, d, J = 6.4 Hz), 7.16-7.27 (4H, m), 7.27-7.34 (3H, m), 7.71 (1H, |
| | d, J = 6.1 Hz) |
| 71 | 1.05-1.23 (4H, m), 1.39-1.53 (2H, m), 1.75 (2H, d, J = 4.3 Hz), 2.28 (3H, s), 3.32 (3H, s), |
| | 3.35 (2H, s), 3.73 (2H, d, J = 6.5 Hz), 4.15 (2H, s), 5.50 (1H, t, J = 6.7 Hz), 5.72 (1H, d, J = 2.8 |
| | Hz), 5.89 (1H, d, J = 2.5 Hz), 6.02 (1H, t, J = 6.8 Hz), 6.24 (1H, dd, J = 7.2, 1.6 Hz), 6.49-6.60 |
| | (1H, m), 7.06-7.18 (3H, m), 7.19-7.30 (3H, m), 11.32 (1H, s) |
| 72 | 1.08 (2H, qd, J = 12.4, 4.4 Hz), 1.45 (2H, dd, J = 4.6, 1.9 Hz), 1.56 (2H, d, J = 12.9 Hz), 1.60- |
| | 1.70 (1H, m), 1.75 (2H, d, J = 4.1 Hz), 2.25 (3H, s), 2.26-2.36 (1H, m), 3.23 (2H, s), 3.24- |
| | 3.29 (2H, m), 3.29 (2H, s), 3.64 (3H, dd, J = 8.4, 5.7 Hz), 3.85 (2H, dd, J = 10.2, 3.9 Hz), 3.89- |
| | 3.95 (4H, m), 4.07 (2H, s), 5.64 (1H, d, J = 2.7 Hz), 5.83 (1H, dd, J = 2.8, 0.9 Hz), 6.27 (1H, t, |
| | J = 6.3 Hz), 6.48 (2H, s), 6.69 (1H, d, J = 6.9 Hz), 7.01 (1H, d, J = 5.9 Hz), 7.19 (1H, t, J = 8.0 |
| | Hz), 7.31 (1H, d, J = 8.4 Hz), 7.72 (1H, d, J = 6.1 Hz) |
| 73 | 1.47 (2H, d, J = 3.2 Hz), 1.80 (2H, d, J = 4.1 Hz), 3.16-3.23 (2H, m),, 3.24 (3H, s), 3.30 (5H, |
| | s), 3.43 (4H, t, J = 5.4 Hz), 3.49-3.54 (2H, m), 3.92 (2H, d, J = 6.3 Hz), 4.13 (2H, s), 5.77 (1H, |
| | d, J = 2.5 Hz), 5.87 (1H, dd, J = 7.5, 2.6 Hz), 6.29-6.44 (2H, m), 6.49 (2H, s), 6.74 (1H, d, J = |
| | 7.6 Hz), 6.91 (1H, d, J = 6.2 Hz), 7.19 (1H, t, J = 8.0 Hz), 7.31 (1H, d, J = 8.3 Hz), 7.52 (1H, d, |
| | J = 7.5 Hz), 7.71 (1H, d, J = 6.1 Hz) |
| 74 | 1.40-1.56 (4H, m), 1.75-1.86 (4H, m), 2.96 (2H, t, J = 11.1 Hz), 3.07-3.12 (4H, m), 3.28- |
| | 3.31 (8H, m), 3.53-3.71 (8H, m), 3.92 (2H, d, J = 6.0 Hz), 4.13 (2H, s), 5.77 (1H, d, J = 2.5 |
| | Hz), 5.88 (1H, dd, J = 7.5, 2.6 Hz), 6.22-6.37 (2H, m), 6.50 (2H, s), 6.74 (1H, d, J = 7.9 Hz), |
| | 6.91 (1H, d, J = 6.2 Hz), 7.20 (1H, t, J = 7.8 Hz), 7.31 (1H, d, J = 8.3 Hz), 7.52 (1H, d, J = 7.6 |
| | Hz), 7.71 (1H, d, J = 5.9 Hz) |
| 75 | 1.45 (2H, d, J = 3.4 Hz), 1.72-1.82 (4H, m), 1.83-1.93 (4H, m), 2.00 (2H, t, J = 7.1 Hz), 2.19- |
| | 2.28 (2H, m), 3.27-3.30 (5H, m), 3.89 (2H, d, J = 6.0 Hz), 4.00 (1H, q, J = 8.2 Hz), 4.12 (2H, |
| | s), 5.76 (1H, d, J = 2.7 Hz), 5.88 (1H, dd, J = 7.5, 2.7 Hz), 6.28-6.38 (1H, m), 6.48 (3H, d, J = |
| | 6.6 Hz), 6.72 (1H, d, J = 7.5 Hz), 6.91 (1H, d, J = 6.0 Hz), 7.19 (1H, t, J = 8.0 Hz), 7.31 (1H, d, |
| | J = 8.2 Hz), 7.52 (1H, d, J = 7.5 Hz), 7.71 (1H, d, J = 6.0 Hz) |
| 76 | 1.22 (3H, s), 1.48 (2H, d, J = 3.2 Hz), 1.81 (2H, d, J = 3.7 Hz), 3.24 (2H, d, J = 5.9 Hz), 3.30 |
| | (3H, s), 3.35 (2H, s), 3.93 (2H, d, J = 6.3 Hz), 4.12-4.18 (4H, m), 4.45 (2H, d, J = 5.6 Hz), |
| | 5.77 (1H, d, J = 2.6 Hz), 5.88 (1H, dd, J = 7.5, 2.7 Hz), 6.32 (1H, t, J = 6.3 Hz), 6.50 (2H, s), |
| | 6.63 (1H, t, J = 5.9 Hz), 6.75 (1H, d, J = 7.6 Hz), 6.88 (1H, d, J = 6.2 Hz), 7.20 (1H, t, J = 8.0 |
| | Hz), 7.31 (1H, d, J = 8.3 Hz), 7.52 (1H, d, J = 7.5 Hz), 7.69 (1H, d, J = 6.1 Hz) |
| 77 | 1.14 (2H, qd, J = 12.4, 4.8 Hz), 1.46 (2H, d, J = 3.2 Hz), 1.56 (2H, d, J = 13.0 Hz), 1.67 (1H, d, |
| | J = 3.9 Hz), 1.75-1.85 (2H, m), 2.95 (2H, t, J = 6.1 Hz), 3.23 (2H, d, J = 11.4 Hz), 3.30 (5H, s), |
| | 3.84 (2H, dd, J = 11.3, 2.5 Hz), 3.92 (2H, d, J = 6.1 Hz), 4.13 (2H, s), 5.77 (1H, d, J = 2.6 Hz), |
| | 5.88 (1H, dd, J = 7.5, 2.7 Hz), 6.32 (1H, t, J = 6.3 Hz), 6.42 (1H, t, J = 5.6 Hz), 6.50 (2H, s), |
| | 6.75 (1H, d, J = 7.7 Hz), 6.89 (1H, d, J = 6.2 Hz), 7.20 (1H, t, J = 7.9 Hz), 7.31 (1H, d, J = 8.2 |
| | Hz), 7.52 (1H, d, J = 7.6 Hz), 7.70 (1H, d, J = 6.0 Hz) |
| 78 | 1.33 (3H, s), 1.40-1.53 (4H, m), 1.78 (2H, d, J = 3.4 Hz), 2.04-2.15 (2H, m), 3.30 (3H, s), |
| | 3.38 (2H, s), 3.48-3.59 (4H, m), 3.91 (2H, d, J = 6.1 Hz), 4.13 (2H, s), 5.73-5.79 (2H, m), |
| | 5.88 (1H, dd, J = 7.5, 2.7 Hz), 6.23 (1H, t, J = 6.2 Hz), 6.50 (2H, s), 6.75 (1H, d, J = 7.7 Hz), |
| | 6.92 (1H, d, J = 6.2 Hz), 7.20 (1H, t, J = 7.9 Hz), 7.31 (1H, d, J = 8.3 Hz), 7.52 (1H, d, J = 7.6 |
| | Hz), 7.69 (1H, d, J = 6.1 Hz) |
| 79 | 1.47 (2H, d, J = 3.7 Hz), 1.56 (3H, s), 1.81 (2H, d, J = 3.7 Hz), 3.31 (5H, s), 3.92 (2H, d, J = 6.2 |
| | Hz), 4.13 (2H, s), 4.28 (2H, d, J = 6.0 Hz), 4.62 (2H, d, J = 6.1 Hz), 5.77 (1H, d, J = 2.5 Hz), |
| | 5.89 (1H, dd, J = 7.5, 2.5 Hz), 6.27 (1H, t, J = 6.6 Hz), 6.50 (2H, s), 6.76 (2H, d, J = 7.0 Hz), |
| | 6.87 (1H, d, J = 6.2 Hz), 7.20 (1H, t, J = 8.0 Hz), 7.31 (1H, d, J = 8.2 Hz), 7.53 (1H, d, J = 7.5 |
| | Hz), 7.70 (1H, d, J = 6.1 Hz) |
| 80 | 0.91 (6H, s), 1.06 (6H, s), 1.45 (2H, d, J = 3.4 Hz), 1.79 (2H, d, J = 4.4 Hz), 1.88 (1H, s), 3.30 |
| | (3H, s), 3.34 (2H, s), 3.91 (2H, d, J = 5.9 Hz), 4.12 (2H, s), 5.76 (1H, d, J = 2.7 Hz), 5.87 (1H, |
| | dd, J = 7.5, 2.7 Hz), 6.12 (1H, s), 6.29-6.39 (1H, m), 6.50 (2H, s), 6.74 (1H, d, J = 7.6 Hz), |
| | 6.94 (1H, d, J = 6.0 Hz), 7.20 (1H, t, J = 7.7 Hz), 7.31 (1H, d, J = 8.3 Hz), 7.52 (1H, d, J = 7.6 |
| | Hz), 7.69 (1H, d, J = 6.1 Hz) |
| 81 | 1.42-1.49 (2H, m), 1.49-1.66 (4H, m), 1.73-1.85 (4H, m), 1.96-2.09 (2H, m), 2.20-2.36 |
| | (1H, m), 2.96 (2H, q, J = 6.4 Hz), 3.27 (2H, s), 3.30 (3H, s), 3.91 (2H, d, J = 5.9 Hz), 4.13 (2H, |
| | s), 5.77 (1H, d, J = 2.5 Hz), 5.88 (1H, dd, J = 7.4, 2.6 Hz), 6.25-6.35 (1H, m), 6.36 (1H, t, J = |
| | 6.4 Hz), 6.49 (2H, s), 6.75 (1H, d, J = 7.7 Hz), 6.91 (1H, d, J = 6.4 Hz), 7.20 (1H, t, J = 8.0 Hz), |
| | 7.31 (1H, d, J = 8.1 Hz), 7.53 (1H, d, J = 7.6 Hz), 7.70 (1H, d, J = 6.1 Hz) |
| 82 | 1.45 (2H, d, J = 3.7 Hz), 1.68-1.83 (4H, m), 3.14 (2H, q, J = 6.6 Hz), 3.25 (2H, s), 3.30 (3H, |
| | s), 3.48 (2H, t, J = 6.2 Hz), 3.92 (2H, d, J = 6.1 Hz), 4.11 (2H, s), 4.46 (2H, s), 5.77 (1H, d, J = |
| | 2.6 Hz), 5.87 (1H, dd, J = 7.5, 2.6 Hz), 6.35 (2H, t, J = 5.5 Hz), 6.49 (2H, s), 6.74 (1H, d, J = |
| | 7.6 Hz), 6.91 (1H, d, J = 6.1 Hz), 7.13-7.38 (7H, m), 7.53 (1H, d, J = 7.5 Hz), 7.69 (1H, d, J = |
| | 6.0 Hz) |
| 83 | 1.45-1.51 (4H, m), 1.52-1.57 (2H, m), 1.70-1.79 (4H, m), 2.24 (3H, s), 3.23 (2H, s), 3.26 |
| | (2H, s), 3.29 (3H, s), 3.40 (2H, t, J = 7.0 Hz), 3.48-3.64 (4H, m), 3.84 (2H, d, J = 5.8 Hz), 4.08 |
| | (2H, s), 5.64 (1H, d, J = 2.8 Hz), 5.83 (1H, dd, J = 2.8, 0.9 Hz), 6.20 (1H, t, J = 6.2 Hz), 6.47 |
| | (2H, s), 6.71 (1H, d, J = 7.7 Hz), 7.05 (1H, d, J = 6.1 Hz), 7.18 (1H, t, J = 8.0 Hz), 7.29 (1H, d, |
| | J = 8.3 Hz), 7.70 (1H, d, J = 6.1 Hz) |
| 84 | 0.24-0.29 (2H, m), 0.41-0.46 (2H, m), 1.39-1.46 (2H, m), 1.51-1.55 (2H, m), 1.59 (2H, |
| | s), 1.71 (2H, d, J = 4.4 Hz), 2.24 (3H, s), 3.16 (2H, s), 3.23 (2H, s), 3.29 (3H, s), 3.37-3.43 |
| | (2H, m), 3.79 (2H, d, J = 5.6 Hz), 4.07 (2H, s), 5.65 (1H, d, J = 2.8 Hz), 5.82 (1H, dd, J = 2.8, |
| | 0.9 Hz), 6.16 (1H, s), 6.53 (2H, s), 6.72 (1H, d, J = 7.7 Hz), 7.08 (1H, s), 7.20 (1H, t, J = 8.0 |
| | Hz), 7.30 (1H, d, J = 8.3 Hz), 7.72 (1H, s) |
| 85 | 1.53 (6H, m), 1.62 (4H, m), 1.72 (4H, m), 2.24 (3H, s), 3.18 (2H, s), 3.25 (2H, s), 3.29 (3H, s), |
| | 3.38 (2H, t, J = 6.9 Hz), 3.85 (2H, d, J = 6.0 Hz), 4.08 (2H, s), 5.64 (1H, d, J = 2.8 Hz), 5.83 |
| | (1H, d, J = 2.7 Hz), 6.22 (1H, t, J = 6.2 Hz), 6.52 (2H, s), 6.73 (1H, d, J = 7.8 Hz), 7.06 (1H, d, |
| | J = 6.2 Hz), 7.20 (1H, t, J = 8.0 Hz), 7.30 (1H, d, J = 8.3 Hz), 7.70 (1H, d, J = 6.1 Hz) |
| 86 | 1.49-1.58 (2H, m), 1.71 (1H, d, J = 5.8 Hz), 1.77 (1H, d, J = 5.7 Hz), 1.92-2.02 (1H, m), 2.19- |
| | 2.22 (1H, m), 2.24 (3H, s), 3.18-3.23 (1H, m), 3.29 (4H, s), 3.38 (1H, d, J = 7.2 Hz), 3.42- |
| | 3.48 (1H, m), 3.49-3.57 (1H, m), 3.67-3.74 (2H, m), 3.75 (3H, s), 3.76-3.80 (1H, m), 4.00- |
| | 4.06 (1H, m), 4.08 (2H, s), 5.64 (1H, d, J = 2.8 Hz), 5.82 (1H, dd, J = 2.8, 0.9 Hz), 6.27 (1H, t, |
| | J = 6.3 Hz), 6.47 (2H, s), 6.73 (1H, d, J = 7.7 Hz), 6.81 (1H, ddd, J = 8.3, 2.5, 1.1 Hz), 6.87- |
| | 6.91 (2H, m), 7.09 (1H, d, J = 6.2 Hz), 7.20 (1H, t, J = 8.0 Hz), 7.24 (1H, dd, J = 8.3, 7.4 Hz), |
| | 7.30 (1H, d, J = 8.3 Hz), 7.71 (1H, d, J = 6.1 Hz) |
| 87 | 1.22-1.31 (1H, m), 1.38-1.45 (1H, m), 1.49-1.57 (3H, m), 1.60-1.76 (5H, m), 2.24 (3H, |
| | s), 3.19 (1H, dd, J = 7.5, 1.6 Hz), 3.29 (3H, s), 3.32-3.41 (3H, m), 3.43-3.52 (2H, m), 3.69- |
| | 3.76 (1H, m), 3.82-3.91 (1H, m), 4.08 (2H, s), 5.65 (1H, d, J = 2.8 Hz), 5.82 (1H, dd, J = 2.8, |
| | 0.9 Hz), 6.15 (1H, t, J = 6.3 Hz), 6.47 (2H, s), 6.70 (1H, d, J = 7.8 Hz), 7.07 (1H, d, J = 6.1 Hz), |
| | 7.19 (1H, t, J = 8.0 Hz), 7.29 (1H, d, J = 8.3 Hz), 7.71 (1H, d, J = 6.1 Hz) |
| 88 | 1.44 (1H, dd, J = 10.1, 6.8 Hz), 1.52 (1H, dd, J = 10.1, 6.8 Hz), 1.78-1.87 (2H, m), 3.30 (3H, |
| | s), 3.43-3.50 (2H, m), 3.86-4.03 (2H, m), 4.15 (2H, s), 5.68-5.76 (1H, m), 5.77 (1H, d, J = |
| | 2.8 Hz), 5.88 (1H, dd, J = 7.5, 2.7 Hz), 6.12 (1H, t, J = 6.5 Hz), 6.51 (2H, s), 6.73 (1H, d, J = |
| | 7.7 Hz), 6.88 (1H, d, J = 6.2 Hz), 7.18 (1H, t, J = 8.0 Hz), 7.32 (1H, d, J = 8.3 Hz), 7.36-7.44 |
| | (3H, m), 7.48 (1H, d, J = 9.8 Hz), 7.52 (1H, d, J = 7.6 Hz), 7.61 (2H, dd, J = 6.7, 3.0 Hz), 7.65 |
| | (1H, d, J = 6.1 Hz) |
| 89 | 1.49-1.55 (m, 2H), 1.56-1.69 (m, 2H), 1.74 (t, J = 5.3 Hz, 2H), 1.98 (h, J = 7.0 Hz, 1H), 2.06 |
| | (dt, J = 12.9, 6.4 Hz, 1H), 2.24 (s, 3H), 3.26 (dd, J = 7.4, 1.5 Hz, 2H), 3.29 (s, 3H), 3.42-3.59 |
| | (m, 4H), 3.80-3.86 (m, 1H), 3.86-3.92 (m, 1H), 4.08 (s, 2H), 5.64 (d, J = 2.8 Hz, 1H), 5.82 |
| | (dd, J = 2.8, 0.9 Hz, 1H), 6.23 (t, J = 6.3 Hz, 1H), 6.48 (s, 2H), 6.71 (d, J = 7.7 Hz, 1H), 7.06- |
| | 7.09 (m, 1H), 7.19 (t, J = 8.0 Hz, 1H), 7.30 (d, J = 8.3 Hz, 1H), 7.71 (d, J = 6.1 Hz, 1H). |
| 90 | 1.59 (t, J = 4.2 Hz, 2H), 1.77 (t, J = 6.2 Hz, 2H), 2.25 (s, 3H), 2.52-2.55 (m, 3H), 3.28-3.30 |
| | (m, 4H), 3.38 (d, J = 7.3 Hz, 1H), 3.83 (dd, J = 14.5, 6.2 Hz, 1H), 3.92 (dd, J = 14.5, 6.0 Hz, |
| | 1H), 4.10 (s, 2H), 4.45 (dd, J = 10.3, 4.2 Hz, 1H), 4.65 (dd, J = 10.2, 9.2 Hz, 1H), 5.66 (d, J = |
| | 2.8 Hz, 1H), 5.83 (d, J = 2.6 Hz, 1H), 5.97 (dd, J = 9.1, 4.2 Hz, 1H), 6.17 (t, J = 6.2 Hz, 1H), |
| | 6.48 (s, 2H), 6.75 (d, J = 7.8 Hz, 1H), 6.87 (d, J = 8.0 Hz, 1H), 6.92 (ddd, J = 8.3, 7.0, 0.9 Hz, |
| | 1H), 7.10 (d, J = 6.2 Hz, 1H), 7.20 (t, J = 8.0 Hz, 1H), 7.22-7.29 (m, 2H), 7.30 (d, J = 8.6 Hz, |
| | 1H), 7.71 (d, J = 6.1 Hz, 1H). |
| 91 | 1.90 (2H, s), 2.04 (2H, s), 3.32 (3H, s), 3.74 (2H, s), 4.07 (2H, s), 4.23 (2H, s), 5.83 (1H, d, J = |
| | 2.6 Hz), 5.93 (1H, dd, J = 7.5, 2.7 Hz), 6.20 (1H, br s), 6.59 (2H, s), 6.83-6.97 (2H, m), 7.05- |
| | 7.20 (5H, m), 7.24 (1H, t, J = 8.0 Hz), 7.49 (1H, d, J = 8.6 Hz), 7.56 (1H, d, J = 7.6 Hz), 7.79 |
| | (1H, d, J = 6.0 Hz), 9.93 (1H, s) |
| 92 | 1.57 (dd, J = 4.6, 1.8 Hz, 2H), 1.86 (d, J = 4.7 Hz, 2H), 2.26 (s, 3H), 3.30 (s, 3H), 3.52 (s, 2H), |
| | 4.01 (d, J = 6.2 Hz, 2H), 4.15 (s, 2H), 5.70 (d, J = 2.8 Hz, 1H), 5.84 (dd, J = 3.0, 0.9 Hz, 1H), |
| | 6.17 (t, J = 6.5 Hz, 1H), 6.51 (s, 2H), 6.74 (d, J = 7.8 Hz, 1H), 6.99 (d, J = 6.2 Hz, 1H), 7.19- |
| | 7.26 (m, 2H), 7.28 (d, J = 8.7 Hz, 1H), 7.33 (d, J = 8.3 Hz, 1H), 7.66 (d, J = 2.0 Hz, 1H), 7.72 |
| | (d, J = 6.1 Hz, 1H), 8.25 (s, 1H), 8.70 (s, 1H). |
| 93 | 0.60-0.78 (2H, m), 0.82-0.93 (2H, m), 1.56-1.66 (2H, m), 1.78 (1H, tt, J = 8.5, 5.2 Hz), |
| | 1.90-2.01 (2H, m), 3.30 (3H, s), 3.52 (2H, s), 4.18 (4H, s), 5.79 (1H, d, J = 2.7 Hz), 5.89 (1H, |
| | dd, J = 7.5, 2.8 Hz), 6.26 (1H, t, J = 6.6 Hz), 6.50 (2H, s), 6.77 (1H, d, J = 7.7 Hz), 6.92 (1H, d, |
| | J = 6.2 Hz), 7.21 (1H, t, J = 8.0 Hz), 7.32 (1H, d, J = 8.3 Hz), 7.52 (1H, d, J = 7.6 Hz), 7.70 (1H, |
| | d, J = 6.1 Hz), 8.20 (2H, s) |
| 94 | 0.73-0.86 (3H, m), 1.73 (2H, d, J = 4.4 Hz), 1.76-1.87 (3H, m), 1.92-2.02 (1H, m), 2.21 |
| | (3H, s), 3.26 (3H, s), 3.32 (2H, s), 3.57 (2H, d, J = 5.7 Hz), 4.10 (2H, s), 5.64 (1H, d, J = 2.8 |
| | Hz), 5.77-5.84 (2H, m), 6.47 (2H, s), 6.53 (1H, d, J = 8.0 Hz) 7.00 (1H, d, J = 6.1 Hz), 7.10 |
| | (1H, d, J = 8.4 Hz), 7.16 (1H, t, J = 8.0 Hz), 7.31 (2H, dd, J = 8.4, 3.0 Hz), 7.69 (1H, d, J = 6.1 |
| | Hz), 8.15 (1H, d, J = 2.7 Hz) |
| 95 | 0.88-1.02 (4H, m), 1.66 (2H, d, J = 3.3 Hz), 1.91 (2H, d, J = 4.2 Hz), 2.05-2.16 (1H, m), 2.25 |
| | (3H, s), 3.30 (3H, s), 3.42 (2H, s), 4.14 (2H, s), 4.18 (2H, d, J = 6.1 Hz), 5.68 (1H, d, J = 2.6 |
| | Hz), 5.83 (1H, d, J = 2.6 Hz), 6.43-6.55 (3H, m), 6.76 (1H, d, J = 7.6 Hz), 6.96 (1H, d, J = 9.2 |
| | Hz), 7.01 (1H, d, J = 6.1 Hz), 7.17-7.29 (2H, m), 7.32 (1H, d, J = 8.4 Hz), 7.69 (1H, d, J = 6.1 |
| | Hz) |
| 97 | 1.55 (2H, s), 1.86 (2H, d, J = 4.7 Hz), 2.29 (3H, s), 3.32 (3H, s), 3.37 (2H, s), 3.79 (2H, t, J = |
| | 8.7 Hz), 3.95 (2H, d, J = 5.8 Hz), 4.14 (2H, s), 4.27 (2H, t, J = 8.7 Hz), 5.70 (1H, d, J = 2.8 Hz), |
| | 5.87 (1H, d, J = 2.8 Hz), 6.52 (2H, d, J = 4.2 Hz), 6.69-6.79 (2H, m), 7.00 (1H, d, J = 6.2 Hz), |
| | 7.23 (1H, t, J = 8.0 Hz), 7.34 (1H, d, J = 8.3 Hz), 7.75 (1H, d, J = 6.1 Hz) |
| 102 | 0.86 (2H t, J = 11.7 Hz), 1.06-1.26 (5H, m), 1.37 (2H, q, J = 7.3 Hz), 1.57 (3H, s ), 1.65 (6H, |
| | s), 1.86-1.95 (2H, m), 2.12-2.23 (2H, m), 3.51 -3.53 (4H, m), 4.59 (2H, s), 5.65 (1H, s), |
| | 5.84-5.90 (1H, m,), 5.96 (1H, s), 6.48 (2H, d, J = 4.4 Hz), 6.68 (1H, d, J = 7.7 Hz), 7.15-7.18 |
| | (1H, d, J = 6.0 Hz), 7.20 (1H, t, J = 8.0 Hz), 7.33 (1H, d, J = 8.3 Hz), 7.52 (1H, d, J = 7.5 Hz), |
| | 7.72 (1H, d, J = 6.0 Hz) |
| 103 | 0.91 (q, J = 11.9 Hz, 2H), 1.16 (h, J = 11.6 Hz, 3H), 1.55 (d, J = 4.3 Hz, 2H), 1.59-1.75 (m, |
| | 6H), 1.91 (d, J = 4.6 Hz, 2H), 2.03-2.10 (m, 2H), 3.31 (s, 3H), 3.52 (d, J = 4.7 Hz, 4H), 4.60 |
| | (s, 2H), 5.65 (d, J = 2.8 Hz, 1H), 5.86 (dd, J = 7.5, 2.7 Hz, 1H), 5.96 (t, J = 6.0 Hz, 1H), 6.48 (d, |
| | J = 4.4 Hz, 2H), 6.68 (d, J = 7.7 Hz, 1H), 7.16 (d, J = 6.0 Hz, 1H), 7.20 (t, J = 8.0 Hz, 1H), 7.33 |
| | (d, J = 8.3 Hz, 1H), 7.52 (d, J = 7.6 Hz, 1H), 7.73 (d, J = 6.1 Hz, 1H) |
| 104 | 1.43 (2H, dd, J = 4.6, 1.7 Hz), 1.75-1.81 (2H, m), 1.83-1.89 (2H, m), 2.27 (3H, s), 2.61 (2H, |
| | t, J = 6.5 Hz), 2.81 (3H, s), 3.37 (2H, s), 3.91 (2H, s), 4.02-4.06 (2H, m), 4.10 (2H, s), 5.67 |
| | (1H, d, J = 2.8 Hz), 5.82 (1H, dd, J = 2.8, 0.9 Hz), 6.52-6.58 (1H, m), 6.68 (2H, s), 7.01-7.08 |
| | (2H, m), 7.11-7.16 (1H, m), 7.37 (1H, t, J = 7.9 Hz), 7.42 (1H, dd, J = 7.6, 1.1 Hz), 7.78 (1H, |
| | d, J = 6.0 Hz), 7.87 (1H, d, J = 8.2 Hz), 8.56 (1H, s); Three protons under the DMSO water |
| | peak. |
| 105 | 0.84-0.91 (2H, m), 0.96-1.04 (2H, m), 1.64 (2H, d, J = 3.3 Hz), 1.91 (2H, d, J = 3.2 Hz), 2.03- |
| | 2.11 (1H, m), 2.26 (3H, s), 3.31 (3H, s), 3.51 (2H, s), 3.97 (2H, d, J = 5.6 Hz), 4.15 (2H, s), |
| | 5.69 (1H, d, J = 2.6 Hz), 5.84 (1H, d, J = 2.6 Hz), 6.13 (1H, t, J = 6.2 Hz), 6.51 (2H, s), 6.71 |
| | (1H, d, J = 7.9 Hz), 7.07 (1H, d, J = 6.0 Hz), 7.22 (1H, t, J = 8.1 Hz), 7.35 (1H, d, J = 8.5 Hz), |
| | 7.73 (1H, d, J = 6.0 Hz) |

### Biological Methods

### Determination of FXIIa Inhibition

In vitro inhibition of Factor XIIa was determined using an IC₅₀ assay based on standard literature methods (see e.g Baeriswyl et al., ACS Chem. Biol., 2015, 10 (8) 1861; Bouckaert et al., European Journal of Medicinal Chemistry 110 (2016) 181). Human Factor XIIa (Enzyme Research Laboratories) was incubated at 25 °C with the fluorogenic substrate H-DPro-Phe-Arg-AFC (Peptide Protein Science) and various concentrations of the test compound. Protease activity was measured by monitoring the accumulation of liberated fluorescence from the substrate over 5 min at 25 °C. The linear rate of fluorescence increase per minute was expressed as percentage (%) activity. The Km for the cleavage of the substrate by FXIIa was determined by standard transformation of the Michaelis-Menten equation. The compound inhibitor assays were performed at substrate Km concentration. IC₅₀ values were calculated as the concentration of inhibitor giving 50% inhibition (IC₅₀) of the uninhibited enzyme activity (100%). Data acquired from this assay are shown in Table 16 below using the following scale:

| Category | IC₅₀ (nM) |
|---|---|
| AAA | 0.1-1 |
| AA | 1-10 |
| A | 10-100 |
| B | 100-300 |
| C | 300-1,000 |
| D | 1,000-3,000 |
| E | 3,000-10,000 |
| F | 10,000-40,000 |
| G | 40,000-100,000 |

For the test compounds that did not achieve 50% inhibition the following scale is used:

| Category | |
|---|---|
| H1 | >20% inhibition @ 40 μM |
| H2 | >20% inhibition @ 100 μM |
| H3 | >20% inhibition @ 400 μM |

TABLE 8
Human FXIIa data, molecular weight and LCMS data

| Example | Human FXIIa | Average | LCMS |
|---|---|---|---|
| No | IC₅₀ (nM) | Molecular Weight | Mass Ion |
| 1 | G | 398.5 | 399.3 |
| 2 | E | 428.5 | 429.2 |
| 3 | AA | 551.6 | 552.3 |
| 4 | B | 553.7 | 554.4 |
| 5 | A | 515.7 | 516.4 |
| 6 | B | 551.6 | 552.4 |
| 7 | B | 551.7 | 552.4 |
| 8 | A | 549.7 | 550.4 |
| 9 | A | 517.6 | 518.4 |
| 10 | A | 503.6 | 504.3 |
| 11 | A | 503.6 | 526.3 |
| | | | [M + Na]⁺ |
| 12 | A | 523.6 | 523.9 |
| 13 | A | 523.6 | 524.7 |
| 14 | A | 565.7 | 566.3 |
| 15 | AA | 565.7 | 566.6 |
| 16 | A | 558.7 | 559.3 |
| 17 | AA | 580.6 | 581.3 |
| 18 | B | 535.6 | 536.0 |
| 19 | A | 554.6 | 555.1 |
| 20 | A | 557.7 | 558.4 |
| 21 | A | 555.7 | 555.9 |
| 22 | A | 541.7 | 542 |
| 23 | A | 565.7 | 566 |
| 24 | A | 559.7 | 560.3 |
| 25 | A | 559.7 | 560.3 |
| 26 | A | 513.6 | 536.2 |
| | | | [M + Na]⁺ |
| 27 | A | 565.7 | 566.2 |
| 28 | A | 579.7 | 580.2 |
| 29 | A | 518.6 | 519.3 |
| 30 | AA | 550.7 | 551.2 |
| 31 | A | 504.6 | 505.3 |
| 32 | A | 538.6 | 539.4 |
| 33 | A | 538.6 | 539.4 |
| 34 | A | 536.6 | 537.3 |
| 35 | A | 552.6 | 553.3 |
| 36 | B | 550.7 | 551.6 |
| 37 | B | 550.7 | 551.4 |
| 38 | AA | 552.6 | 553.6 |
| 39 | B | 580.7 | 581.4 |
| 40 | A | 532.6 | 533.3 |
| 41 | A | 532.6 | 533.3 |
| 42 | A | 552.7 | 553.3 |
| 43 | AA | 552.7 | 553.3 |
| 44 | AA | 564.7 | 565.6 |
| 45 | AA | 578.7 | 579.2 |
| 46 | A | 558.7 | 559.3 |
| 47 | A | 546.7 | 547.4 |
| 48 | A | 546.7 | 547.4 |
| 49 | A | 578.7 | 579.3 |
| 50 | A | 536.6 | 537.3 |
| 51 | A | 514.6 | 515.3 |
| 52 | AAA | 566.7 | 567.3 |
| 53 | A | 502.6 | 503 |
| 54 | A | 502.6 | 503.3 |
| 55 | H2 | 499.6 | 500.3 |
| 56 | G | 513.6 | 514.3 |
| 57 | AA | 564.7 | 565.9 |
| 58 | F | 514.6 | 515.3 |
| 59 | AA | 576.7 | 577.3 |
| 60 | AA | 550.7 | 551.2 |
| 61 | A | 568.7 | 569.2 |
| 62 | AA | 586.6 | 587.1 |
| 63 | A | 434.5 | 435.6 |
| 64 | A | 594.7 | 595.3 |
| 65 | AA | 564.7 | 565.3 |
| 66 | AA | 552.7 | 553.5 |
| 67 | H2 | 517.6 | 518.3 |
| 68 | H1 | 523.6 | 524.3 |
| 69 | AA | 566.7 | 567.3 |
| 70 | A | 592.7 | 593.4 |
| 71 | G | 515.6 | 516.3 |
| 72 | A | 572.7 | 573.4 |
| 73 | A | 536.6 | 537.3 |
| 74 | A | 662.8 | |
| 75 | A | 528.7 | 529.2 |
| 76 | A | 518.6 | 519.2 |
| 77 | A | 532.6 | 533.2 |
| 78 | A | 532.6 | 533.2 |
| 79 | A | 504.6 | 505.1 |
| 80 | A | 530.7 | 531.2 |
| 81 | A | 516.6 | 517.2 |
| 82 | A | 582.7 | 583.2 |
| 83 | A | 572.7 | 573.3 |
| 84 | A | 542.7 | 542.9 |
| 85 | A | 556.7 | 557.3 |
| 86 | A | 608.7 | 609.2 |
| 87 | A | 578.7 | 579.3 |
| 88 | A | 592.6 | 593.7 |
| 89 | A | 564.6 | 565.2 |
| 90 | AA | 580.7 | 581.8 |
| 91 | A | 526.7 | 527.1 |
| 92 | A | 604.6 | 605.7 |
| 93 | A | 509.6 | 510.2 |
| 94 | A | 522.6 | 523.3 |
| 95 | A | 523.6 | 524.4 |
| 97 | A | 474.6 | 474.9 |
| 102 | B | 529.7 | 530.4 |
| 103 | C | 515.7 | 516.3 |
| 104 | A | 594.7 | 595.7 |
| 105 | AA | 513.6 | 514.3 |

### Determination of Related Protease Inhibition

In vitro inhibition of related proteases was determined using an IC₅₀ assay based on standard literature methods (see e.g. Shori et al., Biochem. Pharmacol., 1992,43, 1209; Bouckaert et al., European Journal of Medicinal Chemistry 110 (2016) 181). Human serine protease enzyme Plasma Kallikrein, KLK1, FXa, Plasmin, Thrombin and Trypsin were assayed for enzymatic activity using an appropriate fluorogenic substrate at Km concentration, FXIa at fixed substrate concentration of 100 μM and various concentrations of the test compound. Protease activity was measured by monitoring the accumulation of liberated fluorescence from the substrate over 5 min at 25 °C. The linear rate of fluorescence increase per minute was expressed as percentage (%) activity. IC₅₀ values were calculated as the concentration of inhibitor giving 50% inhibition of the uninhibited enzyme activity (100%).

Data acquired from this assay are shown in Table 17 using the scale shown in Table 18.

TABLE 9
Enzyme selectivity data

| | IC₅₀ | IC₅₀ | IC₅₀ | IC₅₀ | IC₅₀ | IC₅₀ | IC₅₀ |
|---|---|---|---|---|---|---|---|
| Ex- | (hu- | (hu- | (hu- | (hu- | (hu- | (hu- | (hu- |
| am- | man | man | man | man | man | man | man |
| ple | PKal) | KLK1) | plasmin) | FXa) | FXla) | thrombin) | trypsin) |
| No | nM | nM | nM | nM | nM | nM | nM |
| 5 | G3 | D | E | F | | F | F |
| 20 | G3 | F | F | F | F | G3 | |
| 30 | F | D | F | F | E | G3 | |

TABLE 10
Scale used to present enzyme selectivity data

| Category | IC₅₀ (nM) |
|---|---|
| A | 10-100 |
| B | 100-300 |
| C | 300-1,000 |
| D | 1,000-3,000 |
| E | 3,000-10,000 |
| F | 10,000-40,000 |
| G1 | >4,000 |
| G2 | >20,000 |
| G3 | >40,000 |
| G4 | >100,000 |

1. A compound of Formula (I), 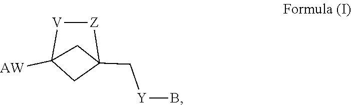
wherein:
V is —O— or —N(R23)-, and Z is —C(R16)(R17)-CH₂— or —C(R16)(R17)-; or,
V is —CH₂—C(R16)(R17)- or —C(R16)(R17)-, and Z is —O— or —N(R18)-;
wherein R18 or R23 are C(═S)NH-aryl; C(═S)NH—R19;
C(═S)N(alkyl)R19; C(═S)NR13R14; C(═O)R19; C(═O)NHR19;
C(═O)N(alkyl)(R19); C(═O)NH₂; heterocycloalkyl^{b}; a 5- or 6-membered carbon-containing aromatic ring containing one, two or three ring members that are, independently, N or, where possible, O, wherein the carbon-containing aromatic ring is substituted with one substituent that is cycloalkyl or heterocycloalkyl^{b}, and optionally one or two further substituents that are, independently, alkyl, alkoxy, OH, OCF₃, halo, CN, or CF₃; 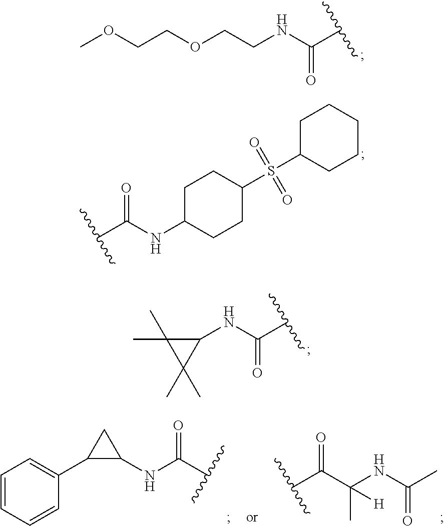
R19 is (CH₂)₁₋₃-cycloalkyl, (CH₂)₀₋₃-polycycloalkyl, heterocycloalkyl^{a}, (CH₂)₁₋₃-heterocycloalkyl^{b}, (CH₂)₀₋₃—R22, (C(R20)(R21))-(CH₂)₀₋₃-phenyl, (C(R20)(R21))-R22, (C(R20)(R21))-O-phenyl, or (CH₂)₁₋₃—O—(CH₂)-phenyl;
R20 and R21 are independently H, alkyl or CF₃, or R20 and R21 together with the carbon atom to which they are attached form a carbon-containing 3-, 4-, 5- or 6-membered saturated ring, optionally containing an O ring member, wherein R20 and R21 are not both H;
R22 is a fused 6,5- or 6,6-bicyclic ring containing an aromatic ring fused to a non-aromatic ring, wherein the bicyclic ring optionally contains one or two ring members that are N or O, wherein the fused 6,5- or 6,6-bicyclic ring is optionally substituted with one or two substituents that are independently alkyl, alkoxy, OH, CN, CF₃, halo, or oxo, wherein the 6,5-bicyclic ring may be attached via the 6- or 5-membered ring, wherein the 6,5-bicyclic ring is not aryl substituted with methylenedioxy, and wherein the 6,6-bicyclic ring is not aryl substituted with ethylenedioxy;
Y is —O— or —N(R12)-;
B is:
(i) heteroaryl^{a}; or
(ii) aryl;
AW— is:
—(CH₂)₀₋₆—(CHR15)-(CH₂)₀₋₆-A, —(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—N(R12)-(CH₂)₁₋₆—C(═O)-A, —(CH₂)₀₋₆—NH—C(═O)—(CH₂)₀₋₆-A, —C(═O)N(R12)-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A or —SO₂—NH-A;
A is a 4- to 15-membered mono-, bi-, or tri-cyclic ring system, containing one N ring member and optionally one, two or three further ring members that are, independently, N, O or S, optionally wherein the ring system is substituted, where possible, with 1, 2, 3 or 4 substituents that are, independently, halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN, or phenyl;
wherein when A is a bicyclic ring system, the bicyclic ring system is fused, bridged or spiro;
wherein when A is a tricyclic ring system, each of the three rings in the tricyclic ring system is either fused, bridged or spiro to at least one of the other rings in the tricyclic ring system;
wherein when:
(i) V is —O—, or
(ii) Z is —O—,
A is A′, wherein A′ is a 4- to 15-membered mono-, bi-, or tri-cyclic ring system, containing one N ring member and optionally one, two or three further ring members that are independently N, O or S, wherein the ring system is substituted with phenyl and optionally, where possible, 1, 2, or 3 further substituents that are independently halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN, or phenyl;
wherein when A′ is a bicyclic ring system, the bicyclic ring system is fused, bridged or spiro;
wherein when A′ is a tricyclic ring system, each of the three rings in the tricyclic ring system is either fused, bridged or spiro to at least one of the other rings in the tricyclic ring system;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy is optionally substituted, where possible, with 1 or 2 substituents that are independently OH, CN, CF₃, or fluoro;
alkyl is a linear saturated hydrocarbon having up to 10 carbon atoms (C₁-C₁₀) or a branched saturated hydrocarbon of between 3 and 10 carbon atoms (C₃-C₁₀); alkyl optionally is substituted with 1, 2 or 3 substituents that are independently alkoxy,
OH, —NR13R14, —C(═O)OR13, —C(═O)NR13R14, CN, CF₃, or halo;
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl^{b} is optionally substituted with 1, 2 or 3 substituents that are independently alkoxy, OH, CN, CF₃, or halo;
small alkyl is a linear saturated hydrocarbon having up to 4 carbon atoms (C₁-C₄) or a branched saturated hydrocarbon of between 3 and 4 carbon atoms (C₃-C₄); small alkyl is optionally substituted with 1 or 2 substituents that are independently alkoxy, OH, NR13R14, C(═O)OR13, C(═O)NR13R14, CN, CF₃, or halo;
aryl is phenyl, biphenyl or naphthyl; aryl is optionally substituted with 1, 2 or 3 substituents that are independently alkyl, alkoxy, methylenedioxy, ethylenedioxy, OH, halo, CN, —(CH₂)₀₋₃—O-heteroaryl^{a}, aryl^{b}, —O-aryl^{b}, —(CH₂)₁₋₃-aryl^{b}, —(CH₂)₀₋₃-heteroaryl^{a}, —C(═O)OR13, —C(═O)NR13R14, —(CH₂)₀₋₃—NR13R14, OCF₃ or CF₃;
aryl^{b} is phenyl, biphenyl or naphthyl; aryl^{b} is optionally substituted with 1, 2 or 3 substituents that are independently alkyl^{b}, alkoxy, OH, halo, CN, or CF₃;
cycloalkyl is a monocyclic saturated hydrocarbon ring of between 3 and 6 carbon atoms (C₃-C₆); cycloalkyl is optionally substituted with 1 or 2 substituents that are independently alkyl, alkoxy, OH, CN, CF₃, or halo;
halo is F, Cl, Br, or I;
heteroaryl is a 5- or 6-membered carbon-containing aromatic ring containing one, two or three ring members that are N, NR8, S, or O; heteroaryl is optionally substituted with 1, 2 or 3 substituents that are independently alkyl, alkoxy, OH, OCF₃, halo, CN, or CF₃;
heteroaryl^{a} is a 5-, 6-, 9- or 10-membered mono- or bi-cyclic aromatic ring, containing, where possible, 1, 2, 3 or 4 ring members that are independently N, N(R12), S or O; heteroaryl^{a} is optionally substituted with 1, 2 or 3 substituents that are independently alkyl, alkoxy, OH, OCF₃, halo, CN, aryl^{b}, —(CH₂)₀₋₃—NR13R14, heteroaryl^{b}, —C(═O)OR12, —C(═O)NR13R14 or CF₃;
heteroaryl^{b} is a 5-, 6-, 9- or 10-membered mono- or bi-cyclic aromatic ring, containing, where possible, 1, 2 or 3 ring members that are independently N, N(R12), S or O; wherein heteroaryl^{b} is optionally substituted with 1, 2 or 3 substituents that are independently alkyl^{b}, alkoxy, OH, halo, CN, aryl^{b}, —(CH₂)₁₋₃-aryl^{b}, or CF₃;
heterocycloalkyl is a non-aromatic carbon-containing monocyclic ring containing 3, 4, 5, or 6, ring members, wherein at least one ring member is independently N, N(R12), S, or O; heterocycloalkyl is optionally substituted with 1 or 2 substituents that are independently alkyl, alkoxy, OH, CN, CF₃, or halo;
heterocycloalkyl^{a} is a 3-10 membered non-aromatic carbon-containing mono- or bi-cyclic ring system containing one or two ring members that are independently N or O; heterocycloalkyl^{a} is optionally substituted with 1 or 2 substituents that are independently alkyl, alkoxy, aryl, halo, OH, CN, CF₃, or heterocycloalkyl; wherein when heterocycloalkyl^{a} is a bicyclic ring system, the bicyclic ring system is spiro;
heterocycloalkyl^{b} is a 3-6 membered non-aromatic carbon-containing monocyclic ring system which may be saturated or, where possible, unsaturated with 1 or 2 double bonds; heterocycloalkyl^{b} contains one or two ring members that are independently N, N(C(═O)CH₃) or O; heterocycloalkyl^{b} is optionally substituted with 1 or 2 substituents that are independently alkyl, alkoxy, halo, OH, CN, CF₃, phenyl or oxo; wherein the point of attachment of heterocycloalkyl^{b} is not at a ring nitrogen;
polycycloalkyl is a bi- or tri-cyclic saturated hydrocarbon ring system of between 5 and 10 carbon atoms (C₅-C₁₀), wherein the bi- or tri-cyclic ring system is bridged or spiro;
R8 is independently, alkyl, cycloalkyl, or heterocycloalkyl;
R12 is independently, alkyl, or cycloalkyl;
R13 and R14 are independently, alkyl^{b}, aryl^{b} or heteroaryl^{b} or R13 and R14 together with the nitrogen atom to which they are attached form a carbon-containing 4-, 5-, 6- or 7-membered heterocyclic ring, optionally containing an additional heteroatom that is N, N(R12), S, SO, SO₂, or O, which may be saturated or unsaturated with 1 or 2 double bonds and which is optionally mono- or di-substituted with substituents that are oxo, alkyl^{b}, alkoxy, OH, halo or CF₃;
R15 is alkyl, halo, CF₃, CN, OH, alkoxy, NR13R14, or CONR13R14;
R16 and R17 are independently H or small alkyl;
or tautomers, isomers, stereoisomers, deuterated analogues, or pharmaceutically acceptable salts and/or solvates thereof.

2. The compound of formula (I) according to claim 1, wherein the compound is a compound of formula (Ia), formula (Ib), or formula (Ic), 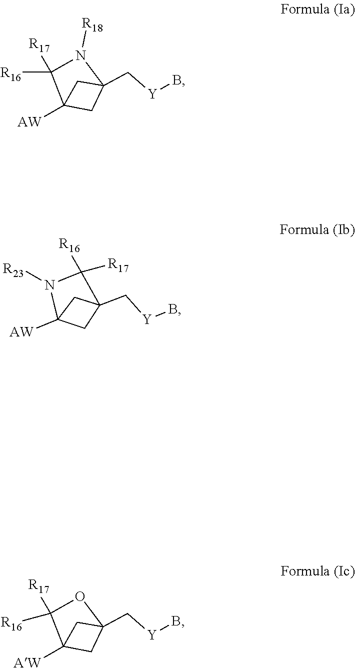
or a tautomer, isomer, stereoisomer, a deuterated analogue, and a pharmaceutically acceptable salt and/or solvate thereof.

3. The compound according to claim 1, or a tautomer, isomer, stereoisomer, a deuterated analogue, and a pharmaceutically acceptable salt and/or solvate thereof, wherein
V is —N(R23)-, and Z is —C(R16)(R17)-CH₂— or —C(R16)(R17)-; or,
V is —CH₂—C(R16)(R17)- or —C(R16)(R17)-, and Z is —N(R18)-.

4. The compound of formula (I) according to claim 1, wherein the compound is a compound of formula (Ia), 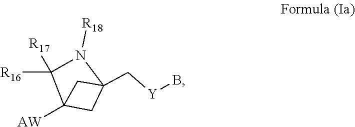
or a tautomer, isomer, stereoisomer, a deuterated analogue, and a pharmaceutically acceptable salt and/or solvate thereof.

5. The compound according to claim 1, or a tautomer, isomer, stereoisomer, a deuterated analogue, and a pharmaceutically acceptable salt and/or solvate thereof, wherein R18 or R23 are C(═S)NH-phenyl; C(═O)R19; C(═O)NHR19; C(═O)N(alkyl)(R19); C(═O)NH₂; heterocycloalkyl^{b}; a 6-membered carbon-containing aromatic ring containing one or two N ring members substituted with one cycloalkyl substituent;

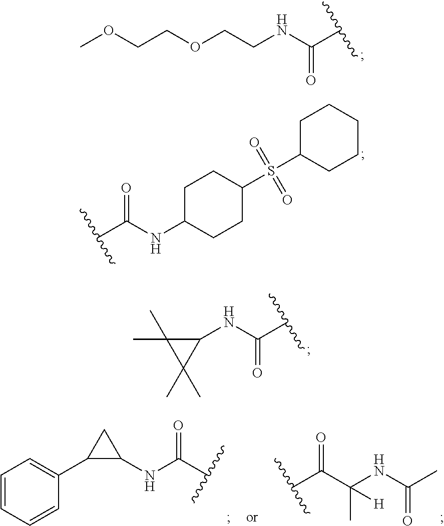

6. The compound according to claim 5, or a tautomer, isomer, stereoisomer, a deuterated analogue, and a pharmaceutically acceptable salt and/or solvate thereof, wherein R18 or R23 are C(═S)NH-phenyl; C(═O)R19; C(═O)NHR19; C(═O)N(alkyl)(R19); C(═O)NH₂; 5-membered heterocycloalkyl^{b} ring, optionally substituted as for heterocycloalkyl^{b}; a 6-membered carbon-containing aromatic ring containing two N ring members substituted with one 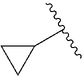 [SMILES: *C1CC1]
substituent;

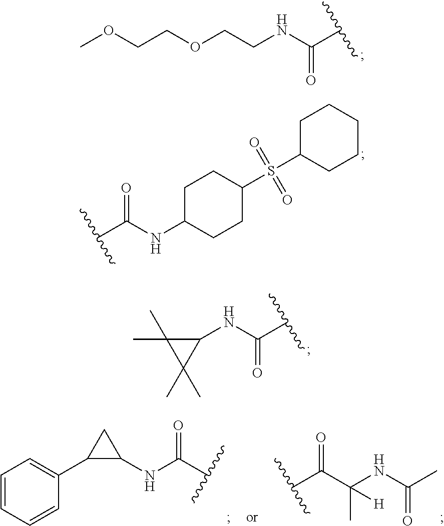

7. The compound according to claim 1, or a tautomer, isomer, stereoisomer, a deuterated analogue, and a pharmaceutically acceptable salt and/or solvate thereof, wherein the compound is a compound of formula (Ia), or a compound of formula (Ib) and R23 is C(═O)R19.

8. The compound according to claim 3, or a tautomer, isomer, stereoisomer, a deuterated analogue, and a pharmaceutically acceptable salt and/or solvate thereof, wherein
A is a 6 to 9 membered mono or bi-cyclic ring system containing one N ring member, optionally wherein the ring system is substituted, where possible, with 1, 2, 3 or 4 substituents that are independently halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, or CN;
wherein when A is a bicyclic ring system, the bicyclic ring system is fused, bridged or spiro.

9. The compound according to claim 8, or a tautomer, isomer, stereoisomer, a deuterated analogue, and a pharmaceutically acceptable salt and/or solvate thereof, wherein A is:

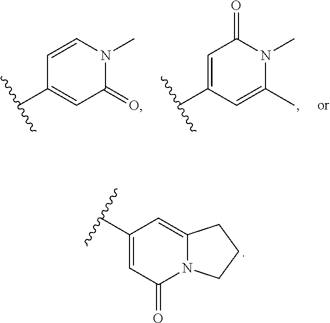

10. The compound according to claim 1, or a tautomer, isomer, stereoisomer, a deuterated analogue, and a pharmaceutically acceptable salt and/or solvate thereof, wherein
V is —O—, and Z is —C(R16)(R17)-CH₂— or —C(R16)(R17)-; or,
V is —CH₂—C(R16)(R17)- or —C(R16)(R17)-, and Z is —O—.

11. The compound according to claim 1, wherein the compound is a compound of formula (Ic), 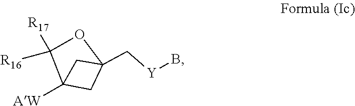
or a tautomer, isomer, stereoisomer, a deuterated analogue, and a pharmaceutically acceptable salt and/or solvate thereof.

12. The compound according to claim 1, or a tautomer, isomer, stereoisomer, a deuterated analogue, and a pharmaceutically acceptable salt and/or solvate thereof, wherein AW— is —(CH₂)₀₋₁-A, or —(CH₂)—O-A.

13. The compound according to claim 1, or a tautomer, isomer, stereoisomer, a deuterated analogue, and a pharmaceutically acceptable salt and/or solvate thereof, wherein Y is —N(R12)-.

14. The compound according to claim 13, or a tautomer, isomer, stereoisomer, a deuterated analogue, and a pharmaceutically acceptable salt and/or solvate thereof, wherein Y is —NH—.

15. The compound according to claim 1, or a tautomer, isomer, stereoisomer, a deuterated analogue, and a pharmaceutically acceptable salt and/or solvate thereof, wherein B is heteroaryl^{a}.

16. The compound according to claim 15, or a tautomer, isomer, stereoisomer, a deuterated analogue, and a pharmaceutically acceptable salt and/or solvate thereof,
wherein B is isoquinolinyl, optionally substituted as for heteroaryl^{a}.

17. The compound according to claim 16 or a tautomer, isomer, stereoisomer, a deuterated analogue, and a pharmaceutically acceptable salt and/or solvate thereof,
wherein B is 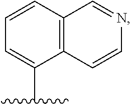 [SMILES: *c1cccc2cnccc12]
optionally substituted as for heteroaryl^{a}.

18. The compound according to claim 16, or a tautomer, isomer, stereoisomer, a deuterated analogue, and a pharmaceutically acceptable salt and/or solvate thereof,
wherein B is isoquinolinyl, substituted with NH₂ at the 1-position 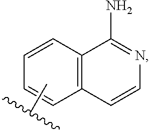
optionally further substituted with 1 or 2 substituents as for heteroaryl^{a}.

19. The compound according to claim 18, or a tautomer, isomer, stereoisomer, a deuterated analogue, and a pharmaceutically acceptable salt and/or solvate thereof,
wherein B is 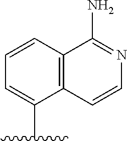 [SMILES: *c1cccc2c(N)nccc12]
optionally further substituted with 1 or 2 substituents as for heteroaryl^{a}.

20. A compound selected from Table 1, Table 2, Table 3, Table 4, and Table 5, or a pharmaceutically acceptable salt, solvate, or solvate of a salt thereof.

21. A pharmaceutical composition comprising a compound, or a pharmaceutically acceptable salt and/or solvate thereof, according to claim 1 and at least one pharmaceutically acceptable excipient.

22. (canceled)

23. A method of treating a disease or condition in which Factor XIIa activity is implicated in a subject in need thereof, wherein the disease or condition in which Factor XIIa activity is implicated is a bradykinin-mediated angioedema, wherein the bradykinin-mediated angioedema is hereditary angioedema, the method comprising administering a compound according to claim 1 to the subject.

24. A method of treating a disease or condition in which Factor XIIa activity is implicated in a subject in need thereof, wherein the disease or condition in which Factor XIIa activity is implicated is a bradykinin-mediated angioedema, wherein the bradykinin-mediated angioedema is non hereditary, the method comprising administering a compound of claim 1 to the subject.

25. A method of treating a disease or condition in which Factor XIIa activity is implicated in a subject in need thereof, wherein the disease or condition in which Factor XIIa activity is implicated is a thrombotic disorder, the method comprising administering a compound according to claim 1 to the subject.

26. The compound according to claim 1, that is an enantiomer, diastereoisomer, or racemic or scalemic mixture thereof.
